(19) United States
(12) Patent Application Publication
(10) Pub. No.: US 2023/0080054 A1
(43) Pub. Date: Mar. 16, 2023

(54) INHIBITORS OF ANOCTAMIN 6 PROTEIN AND USES THEREOF

(71) Applicant: ILDONG PHARMACEUTICAL CO., LTD., Seoul (KR)

(72) Inventors: Jinah JEONG, Hwaseong-Si (KR); Hyunjung KWAK, Hwaseong-Si (KR); Gunhee KIM, Hwaseong-Si (KR); Jeongeun KIM, Hwaseong-Si (KR); Sanghwan LEE, Hwaseong-Si (KR); Seolhee LEE, Hwaseong-Si (KR); Jinhee LEE, Hwaseong-Si (KR); Jihye CHOI, Hwaseong-Si (KR); Hongchul YOON, Hwaseong-Si (KR); Joontae PARK, Hwaseong-Si (KR); Kyungmi AN, Hwaseong-Si (KR); Jungwoo LEE, Hwaseong-Si (KR); Eunjung LEE, Hwaseong-Si (KR)

(21) Appl. No.: 17/580,652

(22) Filed: Jan. 21, 2022

Related U.S. Application Data
(60) Provisional application No. 63/140,695, filed on Jan. 22, 2021.
(60) Provisional application No. 63/141,953, filed on Jan. 26, 2021.

(51) Int. Cl. C07D 405/12; C07D 307/52; C07D 405/14; C07D 237/20; C07D 401/12; C07D 403/12; C07D 409/12; C07C 237/30; C07D 241/20; C07D 239/42; C07D 413/14; C07D 409/14; C07D 471/08; C07D 213/74; C07D 417/12; C07D 271/07; C07D 213/38; C07D 239/26; C07D 241/12; C07D 211/98

(52) U.S. Cl. CPC C07D 405/12; C07D 211/98; C07D 405/14; C07D 237/20; C07D 401/12; C07D 403/12; C07D 409/12; C07C 237/30; C07D 241/20; C07D 239/42; C07D 413/14; C07D 409/14; C07D 471/08; C07D 213/74; C07D 417/12; C07D 271/07; C07D 213/38; C07D 239/26; C07D 241/12; C07D 307/52

(57) ABSTRACT

The present invention relates to a new compound that can inhibit an anoctamin 6 protein, a composition comprising the compound, a method for preparing the compound, and a method for using the compound or composition.

### CROSS REFERENCE TO RELATED APPLICATIONS

This application claims the benefit of U.S. Provisional Application No. 63/140,695, filed Jan. 22, 2021 and U.S. Provisional Application No. 63/141,953, filed Jan. 26, 2021, which applications are hereby incorporated in their entirety by reference.

### TECHNICAL FIELD

The present invention relates to compounds capable of inhibiting anoctamin 6 (ANO6) protein, compositions comprising the compounds, methods for preparing the compounds, and methods of using the compounds or compositions.

### BACKGROUND

ANO6, which is encoded by TMEM16F gene, is a member of a family of transmembrane proteins expressed in a variety of cells. TMEM16F is a Ca2+-gated ion channel that is required for Ca2+-activated phosphatidylserine exposure on the surface of various cells. TMEM16F is widely expressed and has roles in platelet activation during blood clotting, bone formation, and T cell activation. ANO6 has been reported to be essential for phospholipid scrambling required for blood coagulation. It also has been reported to play an important role in controlling cell proliferation and cell death and in occurrence and development of various diseases including hemorrhagic diseases and cancer. See, e.g., Kim et al., TMEM16F forms a Ca²⁺-activated cation channel required for lipid scrambling in platelets during blood coagulation. Cell. 2012; 151(1):111-122; Schreiber et al., Expression and function of epithelial anoctamins. J. Biol. Chem. 2010; 285(10):7838-45; van Kruchten et al., Calcium-activated and apoptotic phospholipid scrambling induced by Ano6 can occur independently of Ano6 ion currents. Cell Death Dis. 2013; 4(4):e611; Xuan et al., ANO6 promotes cell proliferation and invasion in glioma through regulating the ERK signaling pathway. Onco Targets Ther. 2019; 12:6721-6731; and Fan et al., Blockade of phospholipid scramblase 1 with its N-terminal domain antibody reduces tumorigenesis of colorectal carcinomas in vitro and in vivo. J Transl Med. 2012; 10:254, which are incorporated herein by reference.

A need exists for identification of compounds capable of inhibiting ANO6 in order to provide therapeutic agents to treat diseases associated with anoctamin 6 activity, with function of ion channels and/or with function of phospholipid scrambling, including thromboembolic disorder, inflammatory disease, and cancer.

### Definitions

As used herein and in the appended claims, the singular forms “a,” “an,” and “the” include plural referents unless the context clearly dictates otherwise. Thus, for example, reference to “an agent” includes a plurality of such agents, and reference to “a compound” or “the compound” includes reference to one or more compounds and equivalents thereof (e.g., plurality of compounds) known to those skilled in the art, and so forth. The term “about” when referring to a number or a numerical range means that the number or numerical range referred to is an approximation within experimental variability (or within statistical experimental error), and thus the number or numerical range may vary between 1% and 20% of the stated number or numerical range.

Aliphatic hydrocarbon compounds are saturated or unsaturated hydrocarbons based on chains of carbon atoms. They include alkyl, alkenyl, and alkynyl compounds, and their derivatives. The term “alkyl,” when used alone or as part of a larger moiety such as “arylalkyl,” or “cycloalkyl” refers to a straight- or branch-chained, saturated hydrocarbon containing a certain number of carbon atoms (e.g., 1-14 carbon atoms, 1-10 carbon atoms, 1-6 carbon atoms, or 1-4 carbon atoms). For example, “C₁-C₆ alkyl” refers to alkyl having 1 to 6 carbon atoms and is intended to include C₁, C₂, C₃, C₄, C₅, C₆ alkyl groups. Non-limiting examples of alkyl groups include methyl (Me), ethyl (Et), propyl (e.g., n-propyl and iso-propyl), butyl (e.g., n-butyl, iso-butyl, 1-butyl), and pentyl (e.g., n-pentyl, iso-pentyl, neo-pentyl), as well as chain isomers thereof.

The term “alkenyl” when used alone or as part of a larger moiety such as “arylalkenyl,” or “cycloalkenyl” refers to a straight- or branch-chained hydrocarbon containing one or more double bonds and containing a certain number of carbon atoms (e.g., 2-14 carbon atoms, 2-10 carbon atoms, 2-6 carbon atoms, or 2-4 carbon atoms). For example, “C₂-C₆ alkenyl” refers to alkenyl having 2 to 6 carbon atoms and is intended to include C₂, C₃, C₄, C₅, C₆ alkenyl groups. Non-limiting examples of alkenyl groups include ethenyl, propenyl, butenyl, 1-methyl-2-buten-1-yl, heptenyl, octenyl, and the like, as well as chain isomers thereof.

The term “alkynyl” when used alone or as part of a larger moiety such as “arylalkynyl” or “cycloalkynyl” refers to a straight- or branch-chained hydrocarbon containing one or more triple bonds and containing a certain number of carbon atoms (e.g., 2-14 carbon atoms, 2-10 carbon atoms, 2-6 carbon atoms, or 2-4 carbon atoms). For example, “C₂-C₆ alkynyl” refers to alkynyl having 2 to 6 carbon atoms and is intended to include C₂, C₃, C₄, C₅, C₆ alkynyl groups. Non-limiting examples of alkynyl groups include ethynyl, propynyl, butynyl, 1-methyl-2-butyn-1-yl, heptynyl, octynyl, and the like, as well as chain isomers thereof.

Cycloaliphatic hydrocarbon compounds are saturated or unsaturated hydrocarbons containing one (i.e., monocyclic) or more (i.e., polycyclic) non-aromatic rings of carbons. They include cycloalkyl, cycloalkenyl, and cycloalkynyl compounds, and their derivatives. Non-limiting examples of cycloalkyls include cyclopropyl, cyclobutyl, cyclopentyl, cyclohexyl, cycloheptyl, cyclooctyl, cyclodecyl, cyclododecyl, cyclohexenyl, norbornyl,

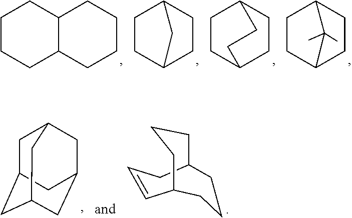

The term “hetero” refers to the replacement of at least one carbon atom member in a ring system with at least one heteroatom such as nitrogen, sulfur, sulfoxide, sulfone, and oxygen. For example, the term “heterocyclo aliphatic” means an aliphatic compound having a non-aromatic monocyclic or polycyclic ring with a certain number of carbons (e.g., 2 to 20 carbon atoms, 2-15 carbon atoms, 2-10 carbon atoms, or 2-7 carbon atoms) in the ring and with one or more heteroatoms selected from nitrogen, oxidized nitrogen (e.g., NO and NO₂), sulfur, oxidize sulfur (e.g., SO and SO₂), and oxygen. The ring or ring system of a heterocyclo aliphatic group of a compound can be linked or fused to one or more different moieties (rings) of the compound via a carbon atom or a heteroatom of the ring. Non-limiting examples of the different ring include a substituted or unsubstituted cycloaliphatic, hetero cycloaliphatic, aromatic, and hetero aromatic ring. A bridged ring may occur when one or more atoms (i.e., C, O, N, or S) link two non-adjacent carbon or nitrogen atoms. Examples of bridged rings include, but are not limited to, one carbon atom, two carbon atoms, one nitrogen atom, two nitrogen atoms, and a carbon-nitrogen group. When a ring is bridged, the substituents recited for the ring may also be present on the bridge.

As used herein, the term “aromatic,” or “aryl” refers to aromatic monocyclic or polycyclic groups. It includes carbocyclic aromatic groups (e.g., phenyl, naphthyl, and the like) and heteroaromatic groups (e.g., pyridyl, pyrimidinyl, and the like). The ring or ring system of an aromatic or heterocyclo aromatic group of a compound can be linked or fused to one or more different moieties (rings) of the compound via at least one carbon atom and/or at least one heteroatom of the ring, which results in fused rings (sharing two adjacent atoms), bridged rings (sharing two non-adjacent atoms), and spiro rings (sharing one atom). Non-limiting examples of the different ring include a substituted or unsubstituted cycloaliphatic, hetero cycloaliphatic, aromatic, and hetero aromatic ring. For example, an aliphatic ring may be fused with an aromatic ring, as illustrated below. The arrowed lines drawn from the illustrated ring system indicate that the bond may be attached to any of the suitable ring atoms.

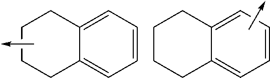

A bridged ring may occur when one or more atoms (e.g., C, O, N, or S) link two non-adjacent carbon, two non-adjacent heteroatoms, or one carbon and one heteroatom. Examples of bridged rings include, but are not limited to, one carbon atom, two carbon atoms, one nitrogen atom, two nitrogen atoms, and a carbon-nitrogen group. When a ring is bridged, the substituents recited for the ring may also be present on the bridge.

Non-limiting examples of heterocyclic groups include azetidinyl, pyrrolidinyl, oxetanyl, imidazolinyl, oxazolidinyl, isoxazolinyl, thiazolidinyl, isothiazolidinyl, tetrahydrofuranyl, piperidyl, piperazinyl, 2-oxopiperazinyl, 2-oxopiperidyl, 2-oxopyrrolodinyl, 2-oxoazepinyl, azepinyl, 4-piperidonyl, tetrahydropyranyl, morpholinyl, thiamorpholinyl, thiomorpholinyl sulfoxide, thiamorpholinyl sulfone, 1,3-dioxolane, tetrahydro-1,1-dioxothienyl, quinuclidinyl, pyridyl, pyrimidinyl, pyrazinyl, pyridazinyl, triazinyl, furyl, quinolyl, isoquinolyl, thienyl, imidazolyl, thiazolyl, indolyl, pyrroyl, oxazolyl, benzofuryl, benzothienyl, benzthiazolyl, isoxazolyl, pyrazolyl, triazolyl, tetrazolyl, thiophenyl, indazolyl, 1,2,4-thiadiazolyl, isothiazolyl, purinyl, carbazolyl, benzimidazolyl, indolinyl, benzodioxolanyl, and benzodioxane.

As used herein, the term “alkoxy” refers to the alkyl groups above bound through oxygen, examples of which include methoxy, ethoxy, iso-propoxy, tert-butoxy, and the like. In addition, alkoxy also refers to polyethers such as —O—(CH₂)₂O—CH₃, and the like.

As used herein, the term “hydroxyalkyl” refers to any hydroxyl derivative of alkyl radical. The term “hydroxyalkyl” includes any alkyl radical having one or more hydrogen atoms replaced by a hydroxy group.

As used herein, the term “aryl aliphatic” refers to aliphatic hydrocarbon compounds having one or more hydrogen atoms replaced by an aryl group. The term “arylalkyl,” or “alkylaryl” includes any alkyl radical having one or more hydrogen atoms replaced by an aryl group, e.g., a benzyl group, a phenethyl group, and the like. The term “arylalkenyl” includes any alkenyl radical having one or more hydrogen atoms replaced by an aryl group. The term “arylalkynyl” includes any alkynyl radical having one or more hydrogen atoms replaced by an aryl group. The term “aryl aliphatic” is meant to include arylalkyl, arylalkenyl, and arylakynyl.

As used herein, the term “amine” refers to a derivative of ammonia in which one, two, or all three hydrogen atoms are replaced by hydrocarbon groups including aliphatic, cycloaliphatic, hetero cycloaliphatic, aromatic, and hetero aromatic. The term “alkyl amine,” or “amine alkyl” refers to ammonia derivative having one, two, or all three hydrogen atoms replaced by an alkyl group. Unless otherwise specified, the term herein includes cyclic amines as well primary, secondary, tertiary amines. Non-limiting examples of amines include, but are not limited to, N(C₂H₅)₂, N(CH₃)₂, N(C₂H₅)(benzyl), methyl piperazine, methyl piperidine, ethyl piperazine, and ethyl piperidine.

As used herein, the term “amide” refers to a carbonyl group bonded to a nitrogen. The simplest example is CONH₂. Non-limiting examples of amines include the ones in which one or two of the hydrogen atoms are replaced by other groups including aliphatic, cycloaliphatic, hetero cycloaliphatic, aromatic, and hetero aromatic.

As used herein, the term “sulfhydryl,” “sulfanyl,” or “thiol” refers to any organosulfur compound containing —SH group. The compounds are in the form R—SH, wherein R represents an aliphatic, aromatic ring or other organic substituent.

Aliphatic, cycloaliphatic, hetero cycloaliphatic, aromatic, heteroaromatic, alkoxy, aryl aliphatic (e.g., arylalkyl), carboxyl, carbonyl, hydroxyl, amine, amide, thioalkyl, and sulfhydryl each independently can be unsubstituted or substituted with one or more suitable substituents.

Non-limiting examples of the substituents include halogen or halogen derivatives (e.g., F, Br, Cl, I, OCHF₂, CF₃, CHF₂, or OCF₃), alkyl, alkenyl, alkynyl, cycloalkyl, cycloalkenyl, cycloalkynyl, hetero cycloalkyl, hetero cycloalkenyl, hetero cycloalkynyl, alkoxy, aryl, aryloxy, diaryl, arylalkyl, arylalkyloxy, cycloalkylalkyl, cycloalkylalkyloxy, amino, hydroxy, hydroxyalkyl, acyl, heteroaryl, heteroaryloxy, heteroarylalkyl, heteroarylalkoxy, aryloxyalkyl, alkylthio, arylalkylthio, aryloxyaryl, alkylamido, alkanoylamino, arylcarbonylamino, nitro, cyano, thiol, haloalkyl, trihaloalkyl, and alkylthio. Also, non-limiting examples of the substituents include ═O, —ORₓ, —SRₓ, ═S, —NRₓR_{y}, —N(alkyl)₃, —NRₓSO₂, —NRₓSO₂R_{y}, —SO₂Rₓ—, —SO₂NRₓR_{y}, —SO₂NRₓCOR_{y}, —SO₃H, —PO(OH)₂, —CORₓ, —COORₓ, COOC(alkyl)₃, —CONRₓR_{y}, —CO(C₁-C₄ alkyl)NRₓR_{y}, —CONRₓ(SO₂)R_{y}, —CO₂(C₁-C₄ alkyl)NRₓR_{y}, —NRₓCOR_{y}, —NRₓCO₂R_{y}, —NRₓ(C₁-C₄ alkyl)CO₂R_{y}, ═N—OH, and ═N—O-alkyl. Rₓ and R_{y} each may be independently selected from hydrogen, alkyl, alkenyl, C₃-C₇ cycloalkyl, C₅-C₁₁ aryl, benzyl, phenylethyl, naphthyl, a 3- to 7-membered heterocycloalkyl, and a 5- to 6-membered heteroaryl.

A “substituent” as used herein refers to a molecular moiety that is covalently bonded to an atom within a molecule of interest. For example, a ring substituent may be a moiety such as a halogen, alkyl group, haloalkyl group or other group that is covalently bonded to an atom (preferably a carbon or nitrogen atom) that is a ring member. Substituents of aromatic groups are generally covalently bonded to a ring carbon atom. The term “substitution” refers to replacing a hydrogen atom in a molecular structure with a substituent, such that the valence on the designated atom is not exceeded, and such that a chemically stable compound (i.e., a compound that can be isolated, characterized, and tested for biological activity) results from the substitution.

When the term “unsaturated” is used herein to refer to a ring or group, the ring or group may be fully unsaturated or partially unsaturated.

As described above, certain groups can be unsubstituted or substituted with one or more suitable substituents by other than hydrogen at one or more available positions, typically 1, 2, 3, 4, or 5 positions, by one or more suitable groups (which may be the same or different). Certain groups, when substituted, are substituted with 1, 2, 3 or 4 independently selected substituents. Suitable substituents include, but are not limited to, halo, alkyl, haloalkyl, aryl, hydroxy, alkoxy, hydroxyalkyl, amino, and the like.

The term “compound” as used herein is meant to include all stereoisomers, geometric isomers, tautomers, isotopes, and prodrug of the chemical structures depicted.

The compounds herein described may have asymmetric centers, geometric centers (e.g., double bond), or both. All chiral, diastereomeric, racemic forms and all geometric isomeric forms of a structure are intended, unless the specific stereochemistry or isomeric form is specifically indicated. In some embodiments, the compounds described herein have one or more chiral centers. It is understood that if an absolute stereochemistry is not expressly indicated, then each chiral center may independently be of the R-configuration or the S-configuration or a mixture thereof. Thus, compounds described herein include enriched or resolved optical isomers at any or all asymmetric atoms as are apparent from the depictions. Racemic mixtures of R-enantiomer and S-enantiomer, and enantio-enriched stereometric mixtures comprising of R- and S-enantiomers, as well as the individual optical isomers can be isolated or synthesized so as to be substantially free of their enantiomeric or diastereomeric partners, and these stereoisomers are all within the scope of the present technology. Compounds of the present disclosure containing an asymmetrically substituted atom may be isolated in optically active or racemic forms. It is well known in the art how to prepare optically active forms, such as by resolution of racemic forms, by synthesis from optically active starting materials, or through use of chiral auxiliaries.

Geometric isomers, resulting from the arrangement of substituents around a carbon-carbon double bond or arrangement of substituents around a cycloalkyl or heterocyclic ring, can also exist in the compounds of the present disclosure. Geometric isomers of olefins, C═N double bonds, or other types of double bonds may be present in the compounds described herein, and all such stable isomers are included in the present disclosure. Specifically, cis and trans geometric isomers of the compounds of the present disclosure may also exist and may be isolated as a mixture of isomers or as separated isomeric forms.

Compounds of the present disclosure also include tautomeric forms. Tautomeric forms result from the swapping of a single bond with an adjacent double bond and the concomitant migration of a proton. Tautomeric forms include prototropic tautomers which are isomeric protonation states having the same empirical formula and total charge. Examples of prototropic tautomers include ketone—enol pairs, amide—imidic acid pairs, lactam—lactim pairs, amide—imidic acid pairs, enamine—imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

The term “prodrug” refers to an agent which is converted into a biologically active drug in vivo by some physiological or chemical process. In some embodiments, a prodrug is converted to the desired drug form, when subjected to a biological system at physiological pH. In some embodiments, a prodrug is enzymatically converted to the desired drug form, when subjected to a biological system. Prodrug forms of any of the compounds described herein can be useful, for example, to provide particular therapeutic benefits as a consequence of an extension of the half-life of the resulting compound in the body, or a reduction in the active dose required. Pro-drugs can also be useful in some situations, as they may be easier to administer than the parent drug. They may, for instance, be bioavailable by oral administration whereas the parent drug is not. The prodrugs may also have improved solubility in pharmacological compositions over the parent drugs. Prodrug forms or derivatives of a compound of this disclosure generally include a promoiety substituent at a suitable labile site of the compound. The promoiety refers to the group that can be removed by enzymatic or chemical reactions, when a prodrug is converted to the drug in vivo. In some embodiments, the promoiety is a group (e.g., a optionally substituted C₁₋₆ alkanoyl, or an optionally substituted C₁₋₆ alkyl) attached via an ester linkage to a hydroxyl group or a carboxylic acid group of the compound or drug.

### SUMMARY

The present invention provides compounds, compositions, and methods that are useful for treating diseases and disorders related to or associated with function of ion channels and/or phospholipid scrambling.

In one aspect, the present invention provides a compound of Formula (I), a pharmaceutically acceptable salt of the compound, a solvate of the compound, or a hydrate of the compound.

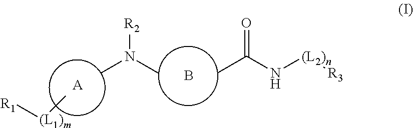

Ring A and ring B each are independently a monocyclic aliphatic ring, a polycyclic aliphatic ring, a monocyclic aromatic ring, or a polycyclic aromatic ring, which optionally contains at least one heteroatom selected from the group consisting of N, NO, NO₂, S, SO, SO₂, and O. The ring A and ring B each may be optionally and independently substituted with at least one substituent selected from the group consisting of halogen, halogen derivatives, CN, alkoxy, carboxyl, carbonyl, ester, hydroxyl, amine, amide, nitro, phosphate, thioalkyl, sulfhydryl, oxo, aliphatic, cycloaliphatic, hetero cycloaliphatic, aromatic, hetero aromatic, and aryl aliphatic.

R₁ and R₃ each are independently hydrogen, halogen, halogen derivatives, CN, alkoxy, carboxyl, carbonyl, hydroxyl, amine, amide, nitro, phosphate, thioalkyl, sulfhydryl, oxo, aliphatic, cycloaliphatic, hetero cycloaliphatic, aromatic, hetero aromatic, or aryl aliphatic. R₁ and R₃ each may be optionally and independently substituted with at least one substituent selected from the group consisting of halogen, halogen derivatives, CN, alkoxy, carboxyl, carbonyl, ester, hydroxyl, amine, amide, nitro, phosphate, thioalkyl, sulfhydryl, oxo, aliphatic, cycloaliphatic, hetero cycloaliphatic, aromatic, hetero aromatic, and aryl aliphatic.

R₂ is hydrogen, C₁₋₅ alkyl, or C₃₋₆ cycloalkyl.

L₁ and L₂ each are independently C₁-C₁₀ aliphatic, C₃-C₁₀ cycloaliphatic, or C₃-C₁₀ hetero cycloaliphatic. L₁ and L₂ each may be optionally and independently substituted with at least one substituent selected from the group consisting of CN, C₁₋₅ alkyl, or C₃₋₆ cycloalkyl.

M and n each are independently 0 or 1.

In another aspect, the present invention provides a composition comprising the compound, the salt, the solvate, the hydrate, or a combination thereof.

In still another aspect, the present invention provides a method of treating or preventing disease, disorder, or condition associated with anoctamin 6 (ANO6) activity, function of ion channels and/or phospholipid scrambling, the method comprising administering to a subject in need a therapeutically effective amount of the compound, salt, solvate, or hydrate or a combination thereof or administering to a subject in need a therapeutically effective amount of the composition comprising the compound, salt, solvate, hydrate, or a combination thereof.

### DETAILED DESCRIPTION

1. Compounds

An aspect of the invention provides compounds, pharmaceutically acceptable salts, solvates, or hydrates thereof. In some embodiments, compounds represented by Formula (I) are provided.

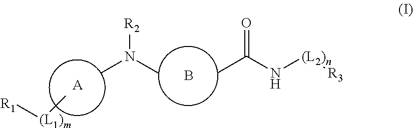

Ring A and ring B each may be independently a monocyclic or polycyclic aliphatic ring or a monocyclic or polycyclic aromatic ring, wherein the aliphatic ring and the aromatic ring each optionally and independently may contain at least one heteroatom selected from the group consisting of N, NO, NO₂, S, SO, SO₂, and O.

R₁, R₂, and R₃ each may be independently hydrogen, halogen, halogen derivatives, CN, alkoxy, carboxyl, carbonyl, hydroxyl, amine, amide, nitro, phosphate, thioalkyl, sulfhydryl, oxo, aliphatic, cycloaliphatic, hetero cycloaliphatic, aromatic, hetero aromatic, or aryl aliphatic.

L₁ and L₂ each may be independently aliphatic, cycloaliphatic, hetero cycloaliphatic, or alkoxy.

M and n each are independently 0 or 1.

The ring A, the ring B, R₁, R₂, R₃, L₁, and L₂ each may be optionally and independently substituted with at least one substituent selected from the group consisting of halogen, halogen derivatives, CN, alkoxy, carboxyl, carbonyl, ester, hydroxyl, amine, amide, nitro, phosphate, thioalkyl, sulfhydryl, oxo, aliphatic, cycloaliphatic, hetero cycloaliphatic, aromatic, hetero aromatic, and aryl aliphatic.

In some embodiments, two or more of the polycyclic rings may be fused or linked with each other.

In some embodiments, the monocyclic or polycyclic aliphatic ring and the monocyclic or polycyclic aromatic ring of the ring A and the ring B each may be independently a 4-membered, 5-membered, 6-membered, 7-membered, 8-membered, 9-membered, 10-membered, 11-membered, or 12-membered ring.

In some embodiments, the monocyclic aliphatic ring and the monocyclic aromatic ring of the ring A may be a 5-membered ring or a 6-membered ring, and the monocyclic aliphatic ring and the monocyclic aromatic ring of the ring B may be a 5-membered ring or a 6-membered ring.

In some embodiments, the monocyclic aliphatic ring and the monocyclic aromatic ring of the ring A may be 5-membered ring or a 6-membered ring, and the monocyclic aliphatic ring and the monocyclic aromatic ring of the ring B may be a 6-membered ring.

In some embodiments, -(L₁)ₘ-R₁ may be connected to the ring A at the para, meta or ortho position. In some embodiments, -(L₁)ₘ-R₁ may be connected to the ring A at the para position.

In some embodiments, the ring A may be a monocyclic or polycyclic aliphatic ring which optionally contains at least one heteroatom selected from the group consisting of N, NO, NO₂, S, SO, SO₂, and O.

In some embodiments, the ring A may be a monocyclic or polycyclic aromatic ring which optionally contains at least one heteroatom selected from the group consisting of N, NO, NO₂, S, SO, SO₂, and O.

In some embodiments, the ring A may be phenyl, pyridinyl, diazinyl, pyrimidinyl, triaziny, piperidinyl, oxadiazoline, cyclopropyl, cyclobutyl, cyclopentyl, or cyclohexyl.

In some embodiments, the ring A may be

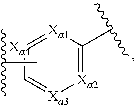

in which Xₐ₁, Xₐ₂, Xₐ₃, and Xₐ₄ each are independently CH, N, NH, NO, or NO₂. In certain embodiments, any one of Xₐ₁, Xₐ₂, Xₐ₃, and Xₐ₄ is N, NH, NO, or NO₂, and the others are CH. In certain embodiments, two of Xₐ₁, Xₐ₂, Xₐ₃, and Xₐ₄ are N, NH, NO, or NO₂, and the others are CH. In certain embodiments, three of Xₐ₁, Xₐ₂, Xₐ₃, and Xₐ₄ are N, NH, NO, or NO₂, and the other one is CH. In certain embodiments, Xₐ₁ and Xₐ₂ are N, and Xₐ₃ and Xₐ₄ are CH. In certain embodiments, Xₐ₁ and Xₐ₃ are N, and Xₐ₂ and Xₐ₄ are CH. In certain embodiments, Xₐ₁ and Xₐ₄ are N, and Xₐ₂ and Xₐ₃ are CH. In certain embodiments, Xₐ₂ and Xₐ₃ are N, and Xₐ₁ and Xₐ₄ are CH. In certain embodiments, Xₐ₂ and Xₐ₄ are N, and Xₐ₁ and Xₐ₃ are CH. In certain embodiments, Xa and Xₐ₄ are N, and Xₐ₁ and Xₐ₂ are CH. In certain embodiments, Xₐ₁, Xₐ₂, and Xₐ₃ are N, and Xₐ₄ is CH.

In some embodiments, the ring A may be

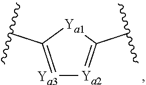

in which Yₐ₁, Yₐ₂, and Yₐ₃ each are independently CH, N, NH, NO, NO₂, S, SH or O. In certain embodiments, any one of Yₐ₁, Yₐ₂, and Yₐ₃ is N, NH, NO, NO₂, S, SH or O, and the others are CH. In certain embodiments, two of Yₐ₁, Yₐ₂, and Yₐ₃ are N, NH, NO, NO₂, S, SH or O, and the other is CH. In certain embodiments, Yₐ₁, and Yₐ₂ are N, NO, NO₂, or NH, and Yₐ₃ is S, SH or O. In certain embodiments, Yₐ₂, and Yₐ₃ are N, NO, NO₂, or NH, and Yₐ₁ is S, SH or O.

In some embodiments, the ring B may be a monocyclic or polycyclic aromatic ring which optionally contains at least one heteroatom selected from the group consisting of N, O, and S.

In some embodiments, the ring B may be a monocyclic or polycyclic aliphatic ring which optionally contains at least one heteroatom selected from the group consisting of N, O, and S.

In certain embodiments, the ring B may be phenyl, pyridinyl, diazinyl, cyclopentadienyl, cyclopentyl, cyclohexyl, adamantane, or bicyclo[2.2.1]heptane.

In some embodiments, the ring B may be

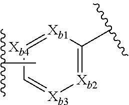

in which X_{b1}, X_{b2}, X_{b3}, and X_{b4} each are independently CH, N, or NH. In certain embodiments, any one of X_{b1}, X_{b2}, X_{b3}, and X_{b4} is N, NH, NO, or NO₂, and the others are CH. In certain embodiments, two of X_{b1}, X_{b2}, X_{b3}, and X_{b4} are N, NH, NO, or NO₂, and the others are CH. In certain embodiments, three of X_{b1}, X_{b2}, X_{b3}, and X_{b4} are N, NH, NO, or NO₂, and the other one is CH. In certain embodiments, X_{b1} and X_{b2} are N, and X_{b3} and X_{b4} are CH. In certain embodiments, X_{b1} and X_{b3} are N, and X_{b2} and X_{b4} are CH. In certain embodiments, X_{b1} and X_{b4} are N, and X_{b2} and X_{b3} are CH. In certain embodiments, X_{b2} and X_{b3} are N, and X_{b1} and X_{b4} are CH. In certain embodiments, X_{b2} and X_{b4} are N, and X_{b1} and X_{b3} are CH. In certain embodiments, X_{b3} and X_{b4} are N, and X_{b1} and X_{b2} are CH. In certain embodiments, X_{b1}, X_{b2}, and X_{b3} are N, and X_{b4} is CH.

In some embodiments, L₁ and L₂ each may be independently C₁-C₁₀ aliphatic, C₃-C₁₀ cycloaliphatic, or C₃-C₁₀ hetero cycloaliphatic. In certain embodiments, L₁ and L₂ each may be independently C₁-C₁₀ aliphatic. In certain embodiments, L₁ and L₂ each may be independently C₁-C₁₀ alkyl or cyclopropyl. In certain embodiments, L₁ and L₂ each may be optionally and independently substituted with at least one substituent selected from the group consisting of halogen, halogen derivatives, CN, alkoxy, hydroxyl, amine, amide, thioalkyl, sulfhydryl, oxo, aliphatic, cycloaliphatic, hetero cycloaliphatic, aromatic, hetero aromatic, and aryl aliphatic. In certain embodiments, L₁ and L₂ each may be optionally and independently substituted with at least one substituent selected from the group consisting of CN, C₁₋₅ alkyl, and C₃₋₆ cycloalkyl.

In some embodiments, R₂ may be hydrogen, C₁₋₅ alkyl or C₃₋₆ cycloalkyl. In certain embodiments, R₂ may be hydrogen or C₁₋₃ alkyl.

In some embodiments, R₁ and R₃ each may be optionally and independently hydrogen, benzyl, amide, amine, thioalkyl, alkoxy, CN, COOH, C₁-C₁₁ aliphatic, C₃-C₁₁ cycloaliphatic, C₃-C₁₁ hetero cycloaliphatic, C₃-C₁₁ aromatic ring, or C₃-C₁₁ hetero aromatic ring.

In some other embodiments, R₁ and R₃ each may be optionally and independently 3-membered cycloaliphatic; 4-membered cycloaliphatic; 4-membered hetero cycloaliphatic; 5-membered cycloaliphatic; 5-membered hetero cycloaliphatic; 6-membered cycloaliphatic; 6-membered hetero cycloaliphatic; 5-membered aromatic ring; 5-membered hetero aromatic ring; 6-membered aromatic ring; 6-membered hetero aromatic ring; 7-membered cycloaliphatic; 7-membered hetero bicyclic aliphatic; 10-membered tricyclic aliphatic; 6-membered aromatic ring fused or linked with 5-membered cycloaliphatic, 5-membered hetero cycloaliphatic, 5-membered aromatic ring, or 5-membered aromatic ring; 6-membered aromatic ring fused or linked with 6-membered cycloaliphatic, 6-membered hetero cycloaliphatic, 6-membered aromatic ring, or 6-membered hetero aromatic ring; 6-membered cycloaliphatic fused or linked with 6-membered cycloaliphatic or 6-membered hetero cycloaliphatic; or 3-membered cycloaliphatic fused or linked with 5-membered aromatic ring, 5-membered hetero aromatic ring, 5-membered cycloaliphatic, 5-membered hetero cycloaliphatic, 6-membered aromatic ring, 6-membered hetero aromatic ring, 6-membered cycloaliphatic, or 6-membered hetero cycloaliphatic, wherein heteroatom is selected from the group consisting of N, O, and S.

In some other embodiments, R₁, and R₃ each may be optionally and independently N(CH₃)₂, N(C₂H₅)₂, N(C₂H₅)(benzyl), or N(C₃H₇)(benzyl).

In some other embodiments, R₁, and R₃ each may be independently hydrogen, C₁₋₁₀ alkyl, C₂₋₅ alkenyl, C₂₋₅ alkynyl, C₃₋₁₁ cycloalkyl, C₃₋₁₁ hetero-cycloalkyl, C₃₋₁₁ cycloalkenyl, C₃₋₁₁ hetero-cycloalkenyl, C₃₋₁₁ cycloalkynyl, C₃₋₁₁ hetero-cycloalkynyl, C₅₋₁₁ aryl, C₅₋₁₁ hetero-aryl, or CN.

In some other embodiments, R₁ may be hydrogen; C₁₋₁₀ alkyl; benzyl; alkoxy; CN; COOH; mono or bi aromatic ring which optionally contains at least one heteroatom selected from the group consisting of N, O, and S; mono or bi cycloaliphatic which optionally contains at least one heteroatom selected from the group consisting of N, O, and S; aryl which optionally contains at least one hetero atom selected from the group consisting of N, O, and S; an aromatic ring fused to a non-aromatic ring which optionally contains at least one heteroatom selected from the group consisting of N, O, and S; or an aromatic ring fused to an aromatic ring which optionally contains at least one heteroatom selected from the group consisting of N, O, and S. R₁ may be substituted or unsubstituted.

In certain embodiments, R₁ may be C₁₋₄ alkyl, benzyl, phenyl, pyridinyl, diazinyl (such as pyrimidinyl, pyrazinyl, and pyridazinyl), triazinyl, piperidinyl, furanyl, cyclopropyl, cyclobutyl, cyclopentyl, cyclohexyl, thiophenyl or oxygen-containing fused heterocycle which is optionally substituted with at least one substituent selected from the group consisting of halogen, halogen derivatives, alkoxy, carboxyl, C₁₋₅ alkyl ester and C₁₋₅ alkyl. In certain embodiments, the substituent is selected from the group consisting of O(CH₃), CH₃, isopropyl, F, C₁, Br, CF₃, NO₂, NH₂, OCHF₂, CHF₂, OCF₃, SCH₃, COOC(CH₃)₃, COOCH₂CH₃, OCH₃, OCH₂CH₃, OCH₂CH₂CH₃, N(C₂H₅)₂, 6-membered hetero cycloaliphatic, dimethyl amine, diethyl amine, and phenyl.

In some embodiments, one of the ring A and R₁ may be or comprise a hetero aromatic ring which contains at least one N as the heteroatom.

In some other embodiments, both of the ring A and R₁ may be or comprise a hetero aromatic ring which contains at least one N as the heteroatom.

In some embodiments, R₃ may be hydrogen, halogen, halogen derivatives, CN, alkoxy, carboxyl, carbonyl, hydroxyl, amine, amide, nitro, phosphate, thioalkyl, sulfhydryl, oxo, aliphatic, cycloaliphatic, hetero cycloaliphatic, aromatic, hetero aromatic, aryl aliphatic or fused ring. In some other embodiments, R₃ may be hydrogen, C₁₋₁₀ alkyl, alkyl amine, mono or bi aromatic ring, mono or bi hetero aromatic ring, mono or bi cycloaliphatic, mono or bi hetero cycloaliphatic, aryl, heteroaryl, aromatic ring fused to a non-aromatic ring which optionally contains at least one heteroatom, or aromatic ring fused to aromatic ring which optionally contains at least one heteroatom. Examples of the heteroatoms include N, O, and S.

In some embodiments, R₃ may be bicycle, cycloaliphatic ring, aryl, or hetero aryl. In some embodiments, R₃ may be C₁₋₁₀ alkyl, alkyl amine, benzyl, COOH, phenyl, pyridinyl, pyrimidinyl, piperidinyl, furanyl, thiophenyl, pyrrolyl, thiazolyl, C₃₋₇ cycloaliphatic, or oxygen-containing fused heterocycle. In some embodiments, R₃ may be optionally substituted with at least one substituent selected from the group consisting of halogen, halogen derivatives, CN, alkoxy, carboxyl, carbonyl, hydroxyl, amine, amide, nitro, phosphate, thioalkyl, sulfhydryl, oxo, aliphatic, cycloaliphatic, hetero cycloaliphatic, aromatic, hetero aromatic, and aryl aliphatic. R₃ may be substituted or unsubstituted.

In some embodiments, R₁, R₂, and R₃ each may be optionally and independently substituted with one or more groups selected from the group consisting of halogen, halogen derivatives (e.g., F, Br, C₁, I, OCHF₂, CF₃, CHF₂, or OCF₃), alkyl, alkenyl, alkynyl, cycloalkyl, cycloalkenyl, cycloalkynyl, hetero cycloalkyl, hetero cycloalkenyl, hetero cycloalkynyl, alkoxy, aryl, aryloxy, diaryl, arylalkyl, arylalkyloxy, cycloalkylalkyl, cycloalkylalkyloxy, amino, hydroxy, hydroxyalkyl, acyl, heteroaryl, heteroaryloxy, heteroarylalkyl, heteroarylalkoxy, aryloxyalkyl, alkylthio, arylalkylthio, aryloxyaryl, alkylamido, alkanoylamino, arylcarbonylamino, nitro, cyano, thiol, haloalkyl, trihaloalkyl, alkyl ester, and alkylthio.

In some embodiments, R₁, R₂, and R₃ each may be optionally and independently substituted with one or more groups selected from ═O, —ORₓ, —SRₓ, ═S, —NRₓR_{y}, —N(alkyl)₃, —NRₓSO₂, —NRₓSO₂R_{y}, —SO₂Rₓ—, —SO₂NRₓR_{y}, —SO₂NRₓCOR_{y}, —SO₃H, —PO(OH)₂, —CORₓ, —COORₓ, COOC(alkyl)₃, —CONRₓR_{y}, —CO(C₁-C₄ alkyl)NRₓR_{y}, —CONRₓ(SO₂)R_{y}, —CO₂(C₁-C₄ alkyl)NRₓR_{y}, —NRₓCOR_{y}, —NRₓCO₂R_{y}, —NRₓ(C₁-C₄ alkyl)CO₂R_{y}, ═N—OH, ═N—O-alkyl. Rₓ and R_{y} each may be independently selected from hydrogen, alkyl, alkenyl, C₃-C₇ cycloalkyl, C₅-C₁₁ aryl, benzyl, phenylethyl, naphthyl, a 3- to 7-membered heterocycloalkyl, and a 5- to 6-membered heteroaryl.

In some embodiments, R₁, and R₃ each may be optionally and independently substituted by at least one substituent selected from the group consisting of O(CH₃), CH₃, CH₂CH₃, isopropyl, F, C₁, Br, CF₃, OCHF₂, CHF₂, OCF₃, SCH₃, COOH, COOC(CH₃)₃, COOCH₂CH₃, COOCH₃, OCH₂CH₃, OCH₂CH₂CH₃, N(C₂H₅)₂, NHCH₃, NO₂, NH₂, CN, dimethyl amine, diethyl amine, phenyl, and 6-membered hetero cycloaliphatic.

In some embodiments, if R₁ is a substituted cyclic compound, the substituent may be bound at the ortho, meta and/or para position of R₁. In some embodiments, the substituent may be bound at the meta, and/or para position of R₁.

In some embodiments, L₂ may be aliphatic, cycloaliphatic, hetero cycloaliphatic, or alkoxy. In some embodiments, L₂ may be C₁₋₅ alkyl or C₁₋₅ cycloaliphatic. In still some other embodiments, L₂ may be C₁₋₃ alkyl or C₁₋₃ cycloaliphatic.

In some embodiments, the

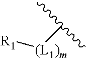

group may be one of the following groups:

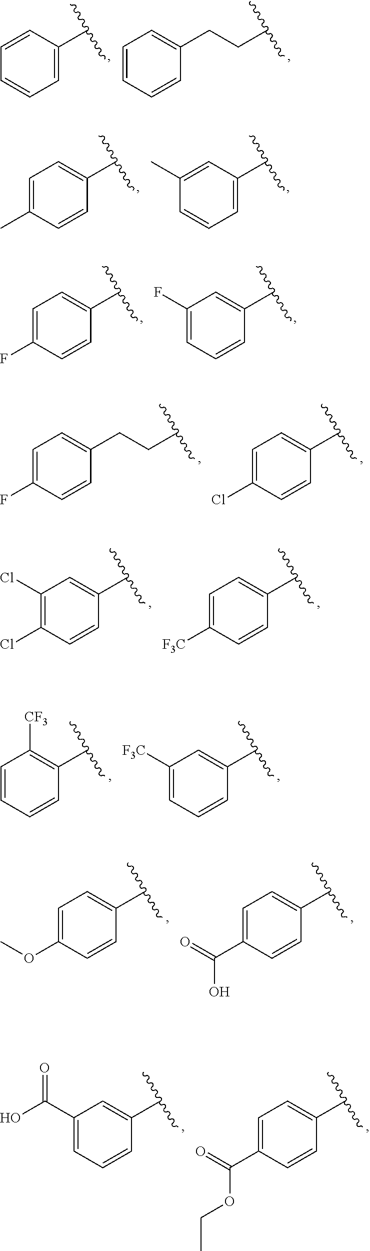

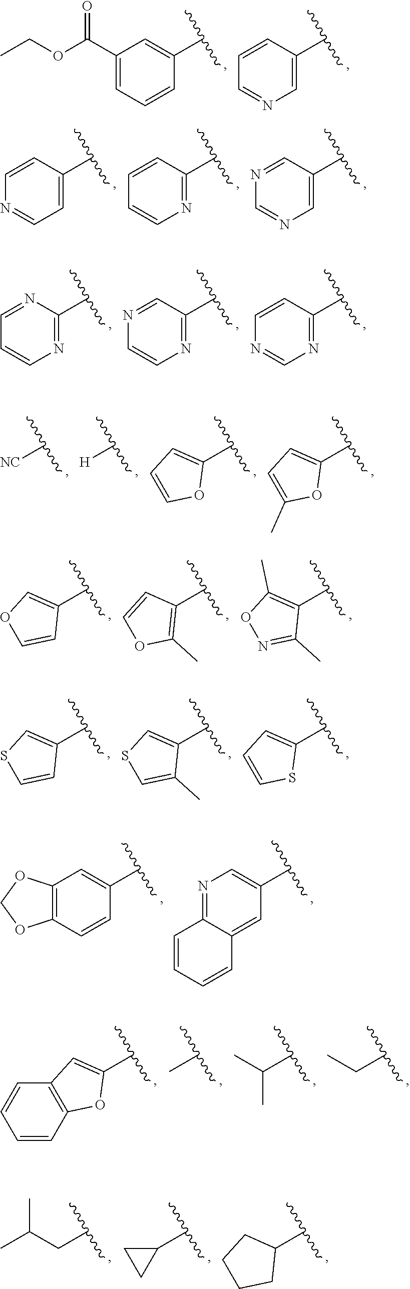

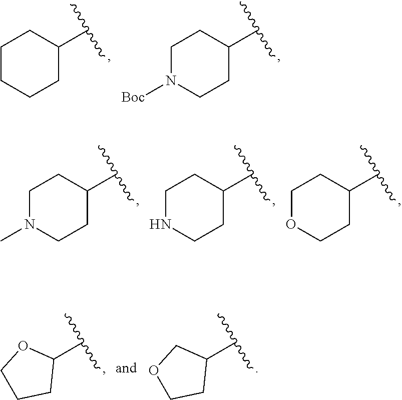

In some embodiments, the

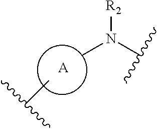

group may be one of the following groups:

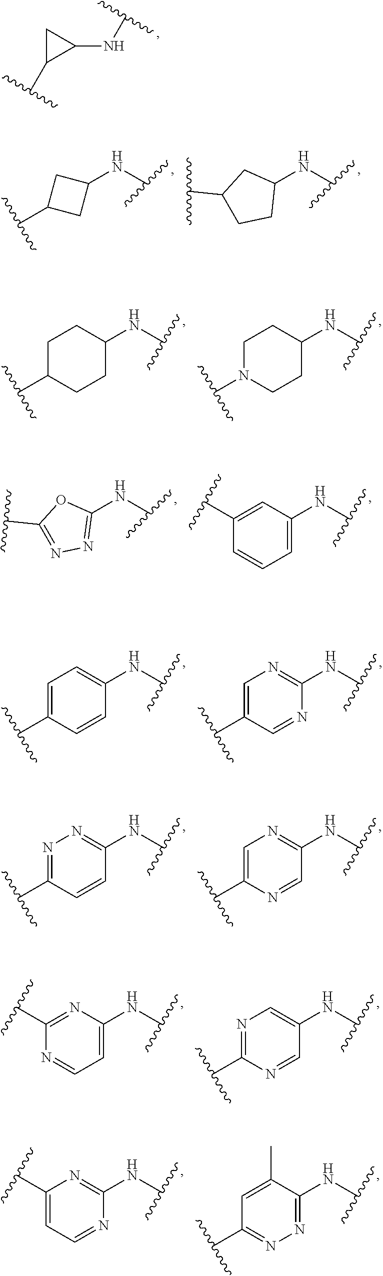

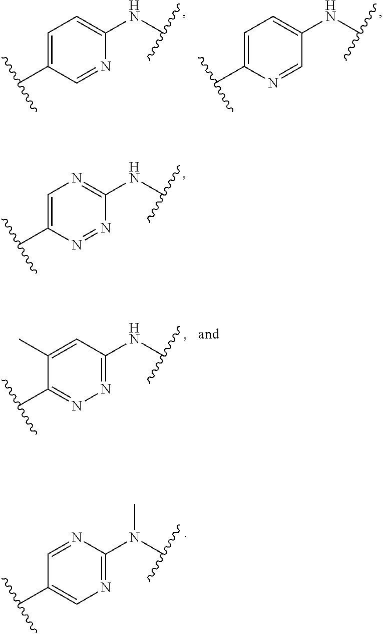

In some embodiments, the

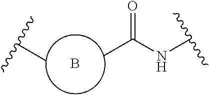

group may be one of the following groups:

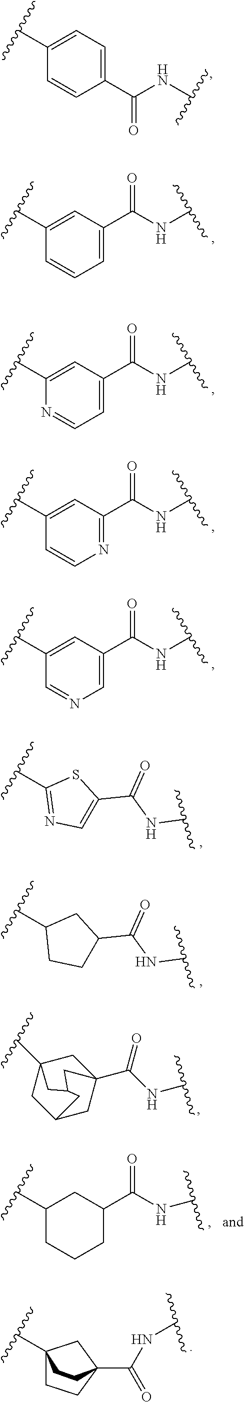

In some embodiments, the

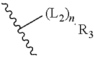

group may be one of the following groups:

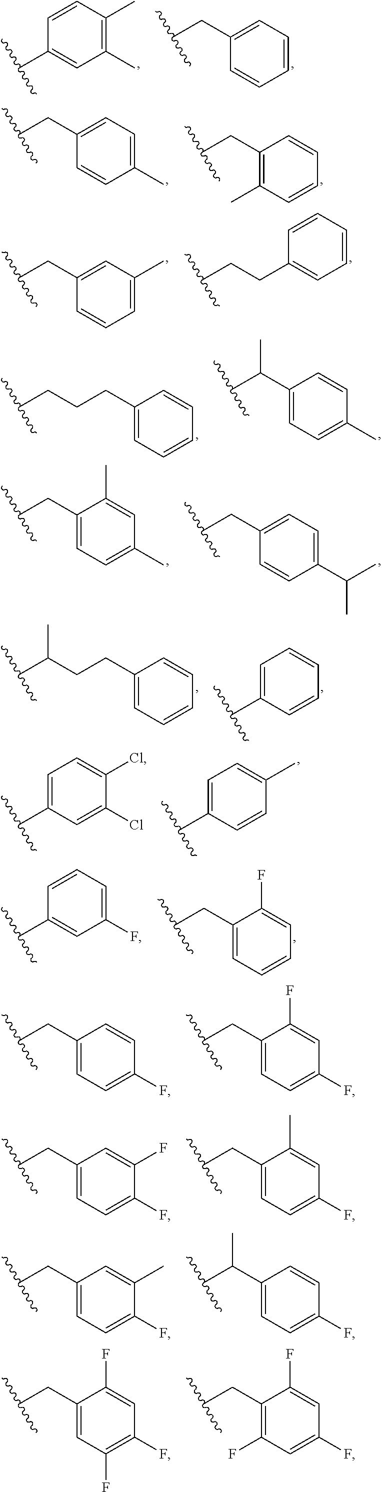

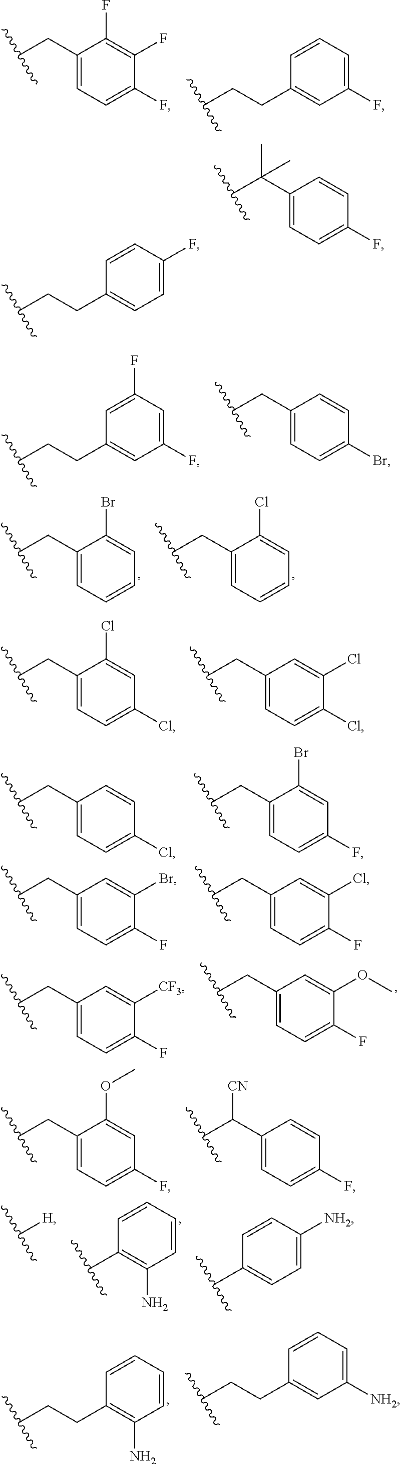

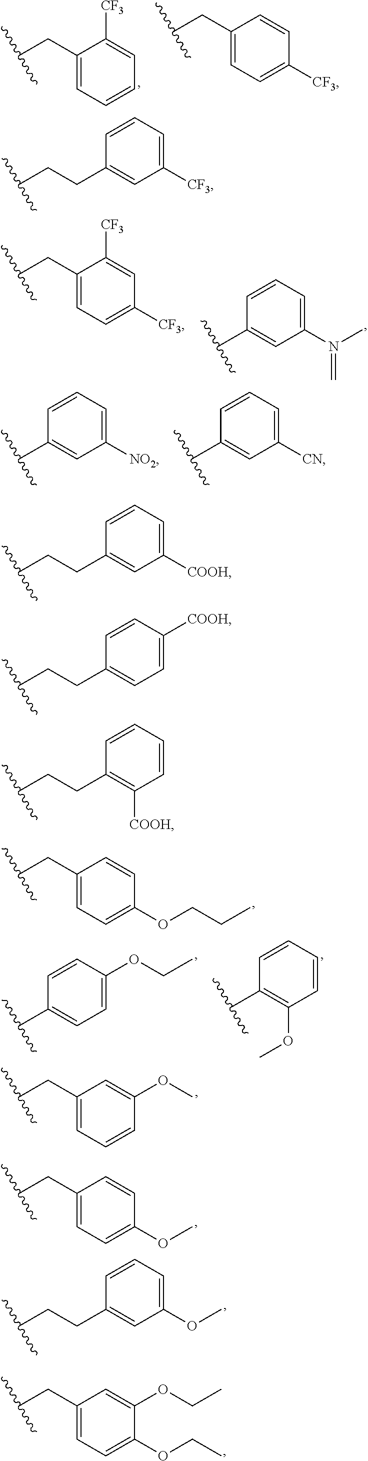

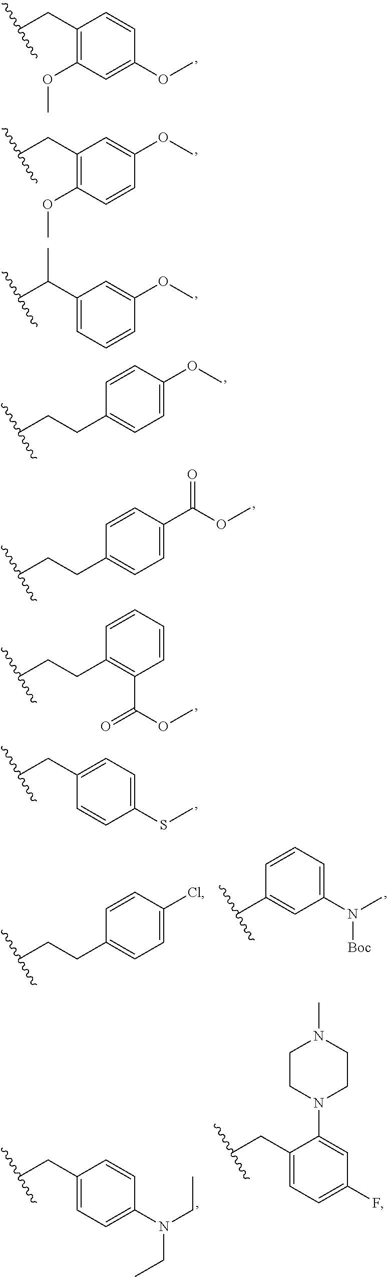

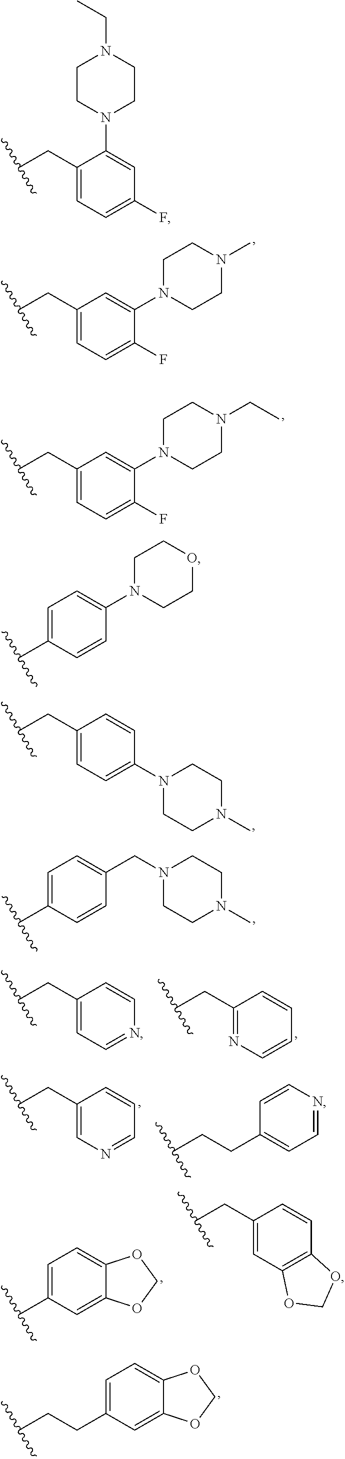

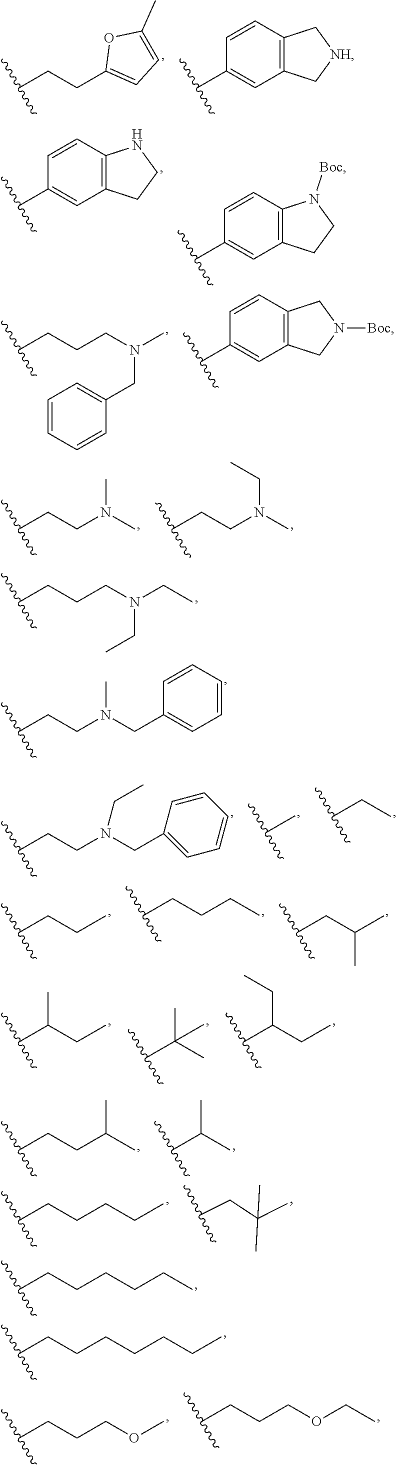

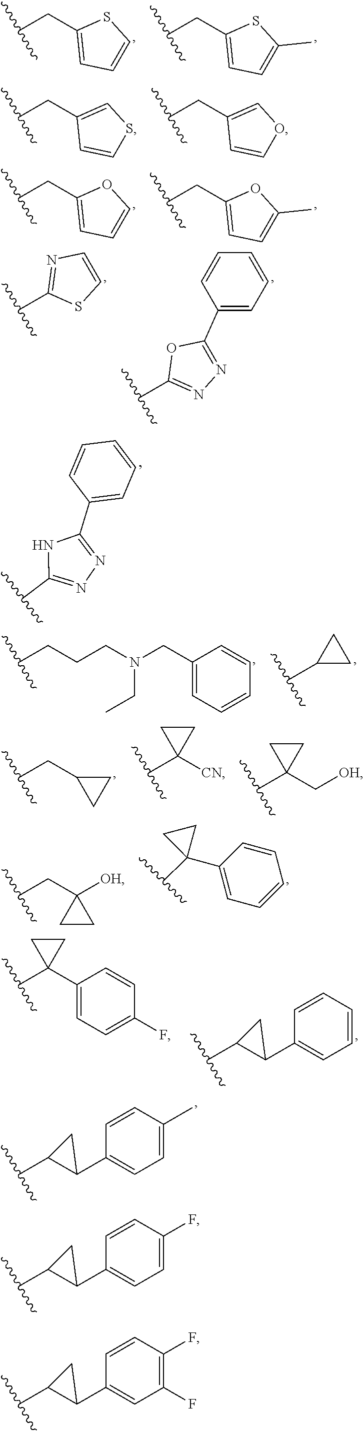

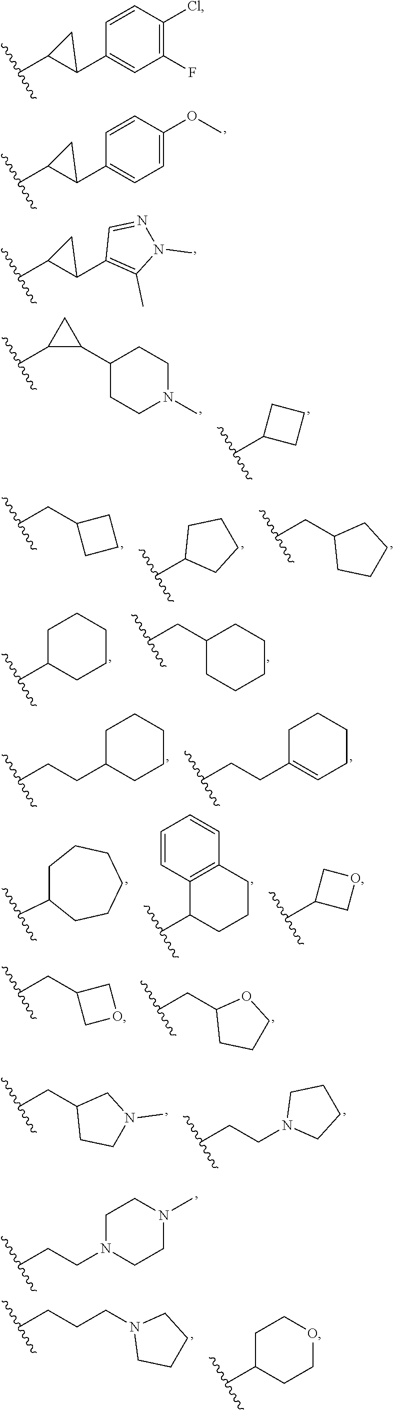

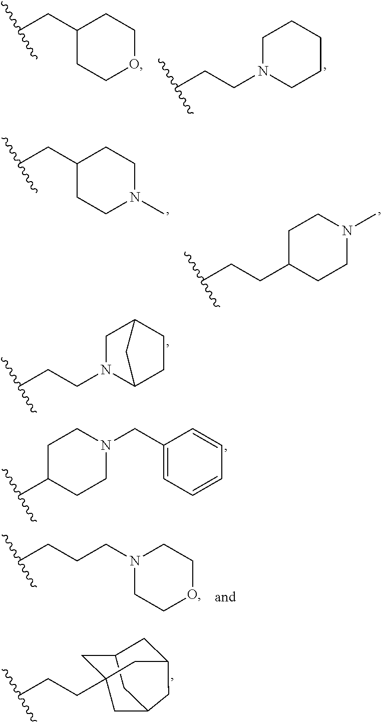

In some other embodiments, compounds represented by Formula (II) are provided.

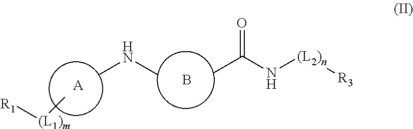

Ring A, Ring B, R₁, R₃, L₁, L₂, m, and n are the same as defined with regard to Formula (I).

In some other embodiments, compounds represented by Formula (III) are provided.

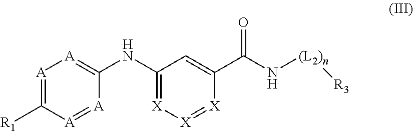

R₁ and R₃ each may be independently hydrogen, halogen, halogen derivatives, CN, alkoxy, carboxyl, carbonyl, hydroxyl, amine, amide, nitro, phosphate, thioalkyl, sulfhydryl, oxo, aliphatic, cycloaliphatic, hetero cycloaliphatic, aromatic, hetero aromatic, or aryl aliphatic. A's and X's each may be independently CH, N, NO, or NH. L₂ may be independently aliphatic, or cycloaliphatic. N may be 0 or 1. A's, R₁, R₃, and L₂ each may be optionally and independently substituted with at least one substituent selected from the group consisting of halogen, halogen derivatives, CN, alkoxy, carboxyl, carbonyl, ester, hydroxyl, amine, amide, nitro, phosphate, thioalkyl, sulfhydryl, oxo, aliphatic, cycloaliphatic, hetero cycloaliphatic, aromatic, hetero aromatic, and aryl aliphatic.

Some embodiments of R₁, R₃, L₂, and n are the same as defined with regard to Formula (I).

In some other embodiments, compounds represented by Formula (IV) are provided.

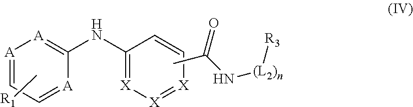

R₁ and R₃ each may be independently hydrogen, halogen, halogen derivatives, CN, alkoxy, carboxyl, carbonyl, hydroxyl, amine, amide, nitro, phosphate, thioalkyl, sulfhydryl, oxo, aliphatic, cycloaliphatic, hetero cycloaliphatic, aromatic, hetero aromatic, or aryl aliphatic. A's and X's each may be independently CH, N, NO, or NH. L₂ may be independently aliphatic, or cycloaliphatic. N may be 0 or 1. A's, R₁, R₃, and L₂ may be optionally and independently substituted with at least one substituent selected from the group consisting of halogen, halogen derivatives, CN, alkoxy, carboxyl, carbonyl, ester, hydroxyl, amine, amide, nitro, phosphate, thioalkyl, sulfhydryl, oxo, aliphatic, cycloaliphatic, hetero cycloaliphatic, aromatic, hetero aromatic, and aryl aliphatic.

Some embodiments of R₁, R₃, L₂, and n are the same as defined with regard to Formula (I).

In some other embodiments, compounds represented by Formula (V) are provided.

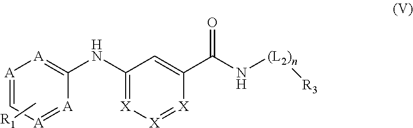

R₁ and R₃ each may be independently hydrogen, halogen, halogen derivatives, CN, alkoxy, carboxyl, carbonyl, hydroxyl, amine, amide, nitro, phosphate, thioalkyl, sulfhydryl, oxo, aliphatic, cycloaliphatic, hetero cycloaliphatic, aromatic, hetero aromatic, or aryl aliphatic. A's and X's each may be independently CH, N, or NH. L₂ may be independently aliphatic, or cycloaliphatic. N may be 0 or 1. A's, R₁, R₃, and L₂ may be optionally and independently substituted with at least one substituent selected from the group consisting of halogen, halogen derivatives, CN, alkoxy, carboxyl, carbonyl, ester, hydroxyl, amine, amide, nitro, phosphate, thioalkyl, sulfhydryl, oxo, aliphatic, cycloaliphatic, hetero cycloaliphatic, aromatic, hetero aromatic, and aryl aliphatic.

Some embodiments of R₁, R₃, L₂, and n are the same as defined with regard to Formula (I).

Non-limiting examples of the compounds of embodiments of the present invention are listed in Table 1 below.

The compounds described herein include all stereoisomers, geometric isomers, tautomers, isotopes, and prodrug of the structures depicted. The compounds described herein can be present in various forms including crystalline, powder and amorphous forms of those compounds, pharmaceutically acceptable salts, including, for example, polymorphs, pseudopolymorphs, solvates, hydrates, unsolvated polymorphs (including anhydrates), conformational polymorphs, and amorphous forms of the compounds, as well as mixtures thereof.

As used herein, the term “pharmaceutically acceptable” refers a material, such as a carrier or diluent, which does not abrogate the biological activity or properties of the compounds described herein. Such materials are administered to an individual without causing undesirable biological effects or interacting in a deleterious manner with any of the components of the composition in which it is contained.

As used herein, the term “pharmaceutically acceptable salt” refers to a formulation of a compound that does not cause significant irritation to an organism to which it is administered and does not abrogate the biological activity and properties of the compounds described herein.

Pharmaceutically acceptable salt forms may include pharmaceutically acceptable acidic/anionic or basic/cationic salts (UK Journal of Pharmaceutical and Biosciences Vol. 2(4), 01-04, 2014, which is incorporated herein by reference). Pharmaceutically acceptable acidic/anionic salts include acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, malonate, mandelate, mesylate, methylsulfate, mucate, napsylate, nitrate, pamoate, pantothenate, phosphate/diphosphate, polygalacturonate, salicylate, stearate, subacetate, succinate, sulfate, hydrogensulfate, tannate, tartrate, teoclate, tosylate, and triethiodide salts. Pharmaceutically acceptable basic/cationic salts include, the sodium, potassium, calcium, magnesium, diethanolamine, N-methyl-D-glucamine, L-lysine, L-arginine, ammonium, ethanolamine, piperazine, and triethanolamine salts.

A pharmaceutically acceptable acid addition salt of a compound of the invention may be prepared by methods known in the art and may be formed by reaction of the free base form of the compound with a suitable inorganic or organic acid including, but not limited to, hydrobromic, hydrochloric, sulfuric, nitric, phosphoric, succinic, maleic, formic, acetic, propionic, fumaric, citric, tartaric, lactic, benzoic, salicylic, glutamic, aspartic, p-toluenesulfonic, benzenesulfonic, methanesulfonic, ethanesulfonic, naphthalenesulfonic such as 2-naphthalenesulfonic, and hexanoic acid. A pharmaceutically acceptable acid addition salt can comprise or be, for example, a hydrobromide, hydrochloride, sulfate, bisulfate, nitrate, phosphate, succinate, maleate, formarate, acetate, propionate, fumarate, citrate, tartrate, lactate, benzoate, carbonate, benzathine, chloroprocaine, choline, histidine, meglumine, meglumine, procaine, triethylamine, besylate, decanoate, ethylenediamine, salicylate, glutamate, aspartate, p-toluenesulfonate, benzenesulfonate, methanesulfonate, ethanesulfonate, naphthalenesulfonate (e.g., 2-naphthalenesulfonate), and hexanoate salt.

A pharmaceutically acceptable base addition salt of a compound of the invention may also be prepared by methods known in the art and may be formed by reaction of the free base form of the compound with a suitable inorganic or organic base including, but not limited to, hydroxide or other salt of sodium, potassium, lithium, ammonium, calcium, magnesium, iron, zinc, copper, manganese, aluminum, tromethamine, glycolate, hydrabamine, methylbromide, methylnitrate, octanoate, oleate, and the like.

A free acid or free base form of a compound of the invention may be prepared by methods known in the art (e.g., for further details see L. D. Bigley, S. M. Berg, D. C. Monkhouse, in “Encyclopedia of Pharmaceutical Technology”. Eds, J. Swarbrick and J. C. Boylam, Vol 13, Marcel Dekker, Inc., 1995, pp. 453-499, which is incorporated herein by reference). For example, a compound of the invention in an acid addition salt form may be converted to the corresponding free base form by treating with a suitable base (e.g., ammonium hydroxide solution, sodium hydroxide, and the like). A compound of the invention in a base addition salt form may be converted to the corresponding free acid by treating with a suitable acid (e.g., hydrochloric acid, etc.).

Aspects of this disclosure include prodrug forms of any of the compounds described herein. Any convenient prodrug forms of the subject compounds can be prepared, for example, according to the strategies and methods described by Rautio et al. (“Prodrugs: design and clinical applications”, Nature Reviews Drug Discovery 7, 255-270 (February 2008)).

Prodrug derivatives of the compounds of the invention may be prepared by methods known to those of ordinary skill in the art (e.g., for further details see Saulnier et al., Bioorg. Med. Chem. Letters, 1994, 4, 1985, which is incorporated herein by reference). Protected derivatives of the compounds of the invention may be prepared by means known to those of ordinary skill in the art. A detailed description of techniques applicable to the creation of protecting groups and their removal can be found in T. W. Greene, “Protecting Groups in Organic Chemistry,” 3^{rd} edition, John Wiley and Sons, Inc., 1999 and “Design of Prodrugs”, ed. 11. Bundgaard, Elsevier, 1985, which are incorporated herein by reference.

The compounds of the present disclosure may be prepared as stereoisomers. Where the compounds have at least one chiral center, they may exist as enantiomers. Where the compounds possess two or more chiral centers, they may exist as diastereomers. The compounds of the invention may be prepared as racemic mixtures. Alternatively, the compounds of the invention may be prepared as their individual enantiomers or diastereomers by reaction of a racemic mixture of the compound with an optically active resolving agent to form a pair of diastereo-isomeric compounds, separating the diastereomers, and recovering the optically pure enantiomers. Resolution of enantiomers may be carried out using covalent diastereomeric derivatives of the compounds of the invention, or by using dissociable complexes (e.g., crystalline diastereomeric salts). Diastereomers have distinct physical properties (e.g., melting points, boiling points, solubility, reactivity, etc.) and may be readily separated by taking advantage of these dissimilarities. The diastereomers may be separated by chromatography, or by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques, Andre Collet and Samuel H. Wilen, “Enantiomers, Racemates and Resolutions” John Wiley And Sons, Inc., 1981, which is incorporated herein by reference.

The compounds of the invention may be prepared as solvates (e.g., hydrates). The term “solvate” refers to a complex of variable stoichiometry formed by a solute (for example, a compound of the invention or a pharmaceutically acceptable salt thereof) and a solvent. Such solvents for the purpose of the invention may not interfere with the biological activity of the solute. Non-limiting examples of suitable solvents include water, acetone, methanol, ethanol and acetic acid. Preferably the solvent used is a pharmaceutically acceptable solvent.

Furthermore, the compounds of the invention may be prepared as crystalline forms. The crystalline forms may exist as polymorphs.

It should be noted that in view of the close relationship between compound of the invention and their other forms, whenever a compound is referred to in this context herein, a corresponding salt, diastereomer, enantiomer, racemate, crystalline, polymorph, prodrug, hydrate, or solvate is also intended, if it is possible or appropriate under certain circumstances.

2. Compositions

Another aspect of the invention provides compositions comprising the compound, pharmaceutically acceptable salt, diastereomer, enantiomer, racemate, solvate, hydrate, prodrug, crystalline, or a combination thereof for use in prevention or treatment of diseases associated with function of ion channels and/or function of phospholipid scrambling.

As used herein, the term “composition” is intended to encompass a product comprising the claimed compound, salt, diastereomer, enantiomer, racemate, hydrate, solvate, or a pharmaceutical combination thereof in the therapeutically effective amount, as well as any other product which results, directly or indirectly, from claimed compound, salt, diastereomer, enantiomer, racemate, hydrate, solvate, or a pharmaceutical combination thereof.

As used herein, the term “pharmaceutical composition” refers to a mixture of a therapeutically active component (ingredient) with one or more other components, which may be chemically or biologically active or inactive. Such components may include, but not limited to, carriers, stabilizers, diluents, dispersing agents, suspending agents, thickening agents, excipients, and adjuvants.

As used herein, the term “pharmaceutical combination” means a product that results from the mixing or combining of more than one therapeutically active ingredient.

As used herein, the term “acceptable” with respect to a formulation, composition or ingredient, as used herein, means having no persistent detrimental effect on the general health of the subject being treated.

As used herein, the term “carrier” refers to chemical or biological material that can facilitate the incorporation of a therapeutically active ingredient(s) into cells or tissues.

Suitable excipients may include, for example, water, pharmaceutically acceptable organic solvents such as paraffins (e.g., petroleum fractions), vegetable oils (e.g. groundnut or sesame oil), mono- or polyfunctional alcohols (e.g., ethanol or glycerol), carriers such as natural mineral powders (e.g., kaoline, clays, talc, chalk), synthetic mineral powders (e.g., highly dispersed silicic acid and silicates), sugars (e.g., cane sugar, lactose and glucose), emulsifiers (e.g., lignin, spent sulphite liquors, methylcellulose, starch and polyvinylpyrrolidone), and lubricants (e.g., magnesium stearate, talc, stearic acid and sodium lauryl sulphate).

Any suitable pharmaceutically acceptable carriers, stabilizers, diluents, dispersing agents, suspending agents, thickening agents, excipients, and adjuvants known to those of ordinary skill in the art for use in pharmaceutical compositions may be selected and employed in the compositions described herein. The compositions described herein may be in the form of a solid, liquid, or gas (aerosol). For example, they may be in the form of tablets (coated tablets) made of, for example, collidone or shellac, gum Arabic, talc, titanium dioxide or sugar, capsules (gelatin), solutions (aqueous or aqueous-ethanolic solution), syrups containing the active substances, emulsions or inhalable powders (of various saccharides such as lactose or glucose, salts and mixture of these excipients with one another), and aerosols (propellant-containing or -free inhale solutions). Also, the compositions described herein may be formulated for sustained or slow release.

3. Methods of Using Compounds or Compositions

Aspects of the present disclosure include methods of treating therapeutic indications of interest using compounds and/or compositions disclosed herein. Therapeutic indications associated with anoctamin 6 activity and/or function of ion channels and/or phospholipid scrambling are referred to herein as “ANO6-related indications.” In some embodiments, methods of the present disclosure may include preventing or treating ANO6-related indications by administering compounds and/or compositions disclosed herein (i.e., ANO6 inhibitors).

ANO6 is a member of a family of transmembrane proteins expressed in a variety of cells. ANO6 acts as both a phospholipid scramblase and ion channels. It has been reported that ANO6 is required for lipid scrambling in platelets during blood coagulation (Kim et al., Cell. 2012; 151(1):111-122).

An ANO6 inhibitor can inhibit anoctamin 6 activity, function of ion channels and/or function of phospholipid scrambling and are a well characterized class of agent having a variety of anti-coagulation activities, anti-cancer (Xuan et al., Onco Targets Ther. 2019; 12:6721-6731; and Fan et al., J Transl Med. 2012; 10:254) and/or anti-inflammation. A human ANO6 inhibition assay can be used to assess the abilities of the compounds of the present disclosure to inhibit target ANO6. In some embodiments, anti-thrombosis, anti-coagulation or anti-blood clotting mean the effect that help prevent, inhibit, or reduce the formation of blood clots (thrombi). ANO6-mediated inhibition activity can determine with a cell-based functional assay utilizing an Example 3 (YFP QUENCHING ASSAY) and Example 4 (LACT C₂ ASSAY). In some embodiments, the administration of the compounds of the present disclosure can cause significant changes of ion channel activity as illustrated by Example 3 (YFP QUENCHING ASSAY) and phosphatidyl serine scramblase activity as illustrated by Example 4 (LACT C₂ ASSAY). In some embodiments, the ANO6 inhibiting compounds of this disclosure have anti-coagulation and anti-thrombotic effects in human blood samples (Example 6; NATEM).

A still another aspect of the invention provides methods for treating or preventing disease, disorder, or condition associated with anoctamin 6 (ANO6) activity, function of ion channels and/or function of phospholipid scrambling. The methods comprise administering to a subject in need a therapeutically effective amount of the compound, pharmaceutically acceptable salt, solvate, hydrate, or a combination thereof or a composition comprising the compound, pharmaceutically acceptable salt, solvate, hydrate, or a combination thereof. Non-limiting examples of the compound are listed in Table 1 and Table 2.

A still another aspect of the invention provides methods for inhibiting anoctamin 6 (ANO6) activity, function of ion channels and/or function of phospholipid scrambling. The methods comprise administering to a subject in need a therapeutically effective amount of the compound, pharmaceutically acceptable salt, solvate, hydrate, or a combination thereof or a composition comprising the compound, pharmaceutically acceptable salt, solvate, hydrate, or a combination thereof. Non-limiting examples of the compound are listed in Table 1 and Table 2.

A still another aspect of the invention provides a composition for treating or preventing disease, disorder, or condition associated with anoctamin 6 (ANO6) activity, function of ion channels and/or function of phospholipid scrambling, comprising the compound, pharmaceutically acceptable salt, solvate, hydrate, or a combination thereof. Non-limiting examples of the compound are listed in Table 1 and Table 2. A still another aspect of the invention provides a composition for inhibiting anoctamin 6 (ANO6) activity, function of ion channels and/or function of phospholipid scrambling.

In some embodiments, the present invention provides a method of treating or preventing disease, disorder, or condition, comprising administering to a subject in need a therapeutically effective amount of the above-described compound, pharmaceutically acceptable salt, solvate, hydrate, or a combination thereof or administering a composition comprising the compound, pharmaceutically acceptable salt, solvate, hydrate or a combination thereof. The method comprises administering to a subject in need a therapeutically effective amount of the compound, pharmaceutically acceptable salt, solvate, hydrate, or a combination thereof or a composition comprising the compound, pharmaceutically acceptable salt, solvate, hydrate, or a combination thereof. Non-limiting examples of the compound are listed in Table 1 and Table 2.

In some other embodiments, the present invention provides a method of treating or preventing disease, disorder, or condition, comprising administering to a subject in need a therapeutically effective amount of a compound listed in Table 1 and Table 2, a pharmaceutically acceptable salt of the compound, a solvate of the compound, a hydrate of the compound, or a composition comprising the compound listed in Table 1 and Table 2, pharmaceutically acceptable salt, solvate, or hydrate.

As used herein, the term “treat,” “treating” or “treatment” refers to methods of alleviating, abating or ameliorating a disease or condition symptoms, preventing additional symptoms, ameliorating or preventing the underlying metabolic causes of symptoms, inhibiting the disease or condition, arresting the development of the disease or condition, relieving the disease or condition, causing regression of the disease or condition, relieving a condition caused by the disease or condition, or stopping the symptoms of the disease or condition either prophylactically and/or therapeutically.

As used herein, the term “subject” or “patient” encompasses mammals and non-mammals. Examples of mammals include, but are not limited to, humans, chimpanzees, apes monkeys, cattle, horses, sheep, goats, swines; rabbits, dogs, cats, rats, mice, guinea pigs, and the like. Examples of non-mammals include, but are not limited to, birds, fishes and the like.

As used herein, the term “administration” or “administering” of the subject compound refers to providing a compound of the invention and/or a prodrug thereof to a subject in need of treatment.

As used herein, the term “effective amount” or “therapeutically effective amount” refer to a sufficient amount of an active ingredient(s) described herein being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. By way of example only, a therapeutically effective amount of a compound of the invention may be in the range of e.g., about 0.01 mg/kg/day to about 1000 mg/kg/day, from about 0.1 mg/kg/day to about 500 mg/kg/day, from about 0.1 mg (×2)/kg/day to about 500 mg (×2)/kg/day.

In addition, such compounds and compositions may be administered singly or in combination with one or more additional therapeutic agents. The methods of administration of such compounds and compositions may include, but are not limited to, intravenous administration, inhalation, oral administration, rectal administration, parenteral, intravitreal administration, subcutaneous administration, intramuscular administration, intranasal administration, dermal administration, topical administration, ophthalmic administration, buccal administration, tracheal administration, bronchial administration, sublingual administration or optic administration. Compounds provided herein may be administered by way of known pharmaceutical formulations, including tablets, capsules or elixirs for oral administration, suppositories for rectal administration, sterile solutions or suspensions for parenteral or intramuscular administration, lotions, gels, ointments or creams for topical administration, and the like. In some embodiments, such pharmaceutical compositions are formulated as tablets, pills, capsules, a liquid, an inhalant, a nasal spray solution, a suppository, a solution, a gel, an emulsion, an ointment, eye drops, or ear drops.

The therapeutically effective amount may vary depending on, among others, the disease indicated, the severity of the disease, the age and relative health of the subject, the potency of the compound administered, the mode of administration and the treatment desired. The required dosage will also vary depending on the mode of administration, the particular condition to be treated and the effect desired.

Specifically, the invention relates to a method of treating or preventing diseases, disorders, or conditions associated with anoctamin 6 (ANO6) activity (ANO6-related indications), function of ion channels and/or function of phospholipid scrambling.

ANO6 inhibitor can prevent of treat diseases, disorders, or conditions associated with anoctamin 6 (ANO6) activity by inhibiting or modulating function of ion channels and/or function of phospholipid scramblase. In specific, inhibition of ANO6 activity can suppresses phosphatidyl serine exposure, thereby inhibiting the formation of tenase complex and prothrombinase complex, and inhibiting thrombin generation, thereby delaying or inhibiting thrombus formation.

In some embodiments, inhibition of anoctamin 6 (ANO6) includes inhibiting ANO6 protein activity. Inhibition of ANO6 suppresses or modulates blood coagulation, and/or cell death by inhibiting the phospholipid scrambling, and thereby can prevent or treat ANO6-related indications.

In this light, the invention provides methods for delaying or inhibiting formation of thrombus, blood clotting, and/or blood coagulation. The methods comprise administering to a subject in need a therapeutically effective amount of the compound, pharmaceutically acceptable salt, solvate, hydrate, or a combination thereof or a composition comprising the compound, pharmaceutically acceptable salt, solvate, hydrate, or a combination thereof. In another aspect of the invention provides a composition for delaying or inhibiting formation of thrombus, blood clotting, and/or blood coagulation comprising the compound, pharmaceutically acceptable salt, solvate, hydrate, or a combination thereof. Non-limiting examples of the compound are listed in Table 1 and Table 2.

In this light, the invention provides methods for inhibiting formation or proliferation of tumor cells. The methods comprise administering to a subject in need a therapeutically effective amount of the compound, pharmaceutically acceptable salt, solvate, hydrate, or a combination thereof or a composition comprising the compound, pharmaceutically acceptable salt, solvate, hydrate, or a combination thereof. In another aspect of the invention provides a composition for inhibiting formation or proliferation of tumor cells comprising the compound, pharmaceutically acceptable salt, solvate, hydrate, or a combination thereof. Non-limiting examples of the compound are listed in Table 1 and Table 2.

In some embodiments, the function of ion channels is meant to comprise dysfunction of ion channels; and hyperactivation of ion channel by dysfunction of ion channels. In some embodiments, the function of phospholipid scrambling is meant to comprise dysfunction of phospholipid scrambling; and hyperactivation of phospholipid scrambling by dysfunction of phospholipid scrambling.

Non-limiting examples of the diseases, disorders, or conditions associated with anoctamin 6 (ANO6) activity, function of ion channels and/or function of phospholipid scrambling may include, but not limited to, thromboembolic disorder, cancer, and inflammatory disease. See, e.g., K. M. Kodigepalli et al., Roles and regulation of phospholipid scramblases. FEBS Letters. 2015; 589(1):3-14, which is incorporated herein by reference.

The term “thromboembolic disorder” as used herein includes arterial cardiovascular thromboembolic disorders, venous cardiovascular thromboembolic disorders, and thromboembolic disorders in the chambers of the heart. The term “thromboembolic disorders” as used herein also includes specific disorders selected from, but not limited to, embolism, thrombosis, pulmonary thromboembolism, unstable angina or other acute coronary syndromes, first or recurrent myocardial infarction, ischemic sudden death, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from (a) prosthetic valves or other implants, (b) indwelling catheters, (c) stents, (d) cardiopulmonary bypass, (e) hemodialysis, (f) infection or (g) other procedures in which blood is exposed to an artificial surface that promotes thrombosis. It is noted that thrombosis includes occlusion (e.g., after a bypass) and reocclusion (e.g., during or after percutaneous transluminal coronary angioplasty). The thromboembolic disorders may result from conditions including but not limited to atherosclerosis, surgery or surgical complications, prolonged immobilization, arterial fibrillation, congenital thrombophilia, cancer, diabetes, effects of medications or hormones, and complications of pregnancy.

Other embodiments and uses will be apparent to one skilled in the art in light of the present disclosures. The following examples are provided merely as illustrative of various embodiments and shall not be construed to limit the invention in any way.

### EXAMPLES

The present invention is further exemplified by the following examples. The examples are for illustrative purpose only and are not intended to limit the invention, nor should they be construed as limiting the invention in any manner. Those skilled in the art will appreciate that variations and modifications can be made without changing the scope of the invention.

¹H and ¹³C NMR spectra were recorded in CDCl₃ (residual internal standard CHCl₃=δ 7.26), DMSO-d₆ (residual internal standard CD₃SOCD₂H=δ 2.50), methanol-d₄ (residual internal standard CD₂HOD=δ 3.20), or acetone-d4 (residual internal standard CD₃COCD₂H=δ 2.05). The chemical shifts (δ) reported are given in parts per million (ppm) and the coupling constants (J) are in Hertz (Hz). The spin multiplicities are reported as s=singlet, bs=broad singlet, bm=broad multiplet, d=doublet, t=triplet, q=quartet, p=pentuplet, dd=doublet of doublet, ddd=doublet of doublet of doublet, dt=doublet of triplet, td=triplet of doublet, tt=triplet of triplet, and m=multiplet.

Medium pressure liquid chromatography (MPLC) was performed with silica gel columns in both the normal phase and reverse phase.

### Example 1: Synthesis of Common Intermediates

In general, compounds used in the reactions described herein may be made according to organic synthesis techniques known to those skilled in this art, starting from commercially available chemicals and/or from compounds described in the chemical literature. “Commercially available chemicals” may be obtained from standard commercial sources including Aldrich Chemical (Milwaukee Wis., including Sigma Chemical and Fluka), Fisher Scientific Co. (Pittsburgh Pa.), and Wako Chemicals USA, Inc. (Richmond Va.), for example.

Methods known to one of ordinary skill in the art may be identified through various reference books and databases. Suitable reference books and treatises that detail the synthesis of reactants useful in the preparation of compounds described herein, or provide references to articles that describe the preparation, include for example, “Synthetic Organic Chemistry,” John Wiley & Sons, Inc., New York; “Advanced Organic Chemistry: Reactions, Mechanisms and Structure”, 4th Ed., Wiley-Interscience, New York, 1992; “Organic Synthesis: Concepts, Methods, Starting Materials,” Second, Revised and Enlarged Edition (1994) John Wiley & Sons; and “Chemistry of Functional Groups” John Wiley & Sons, in 73 volumes.

Specific and analogous reactants may also be identified through the indices of known chemicals prepared by the Chemical Abstract Service of the American Chemical Society, Washington, D.C. Chemicals that are known but not commercially available in catalogs may be prepared by custom chemical synthesis houses.

1. Synthesis of Formula II-a

1) Suzuki Coupling A

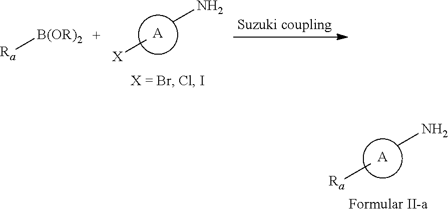

### Synthesis of 6-(3-fluorophenyl)pyridazin-3-amine

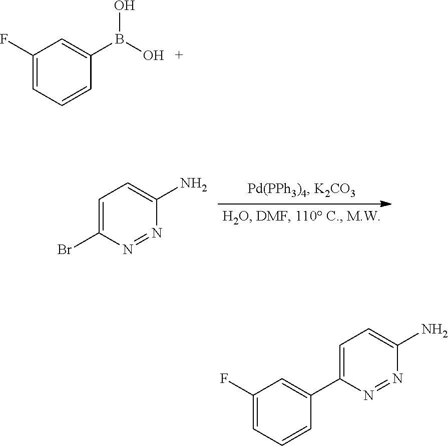

(3-Fluorophenyl)boronic acid (300 mg, 2.15 mmol), 6-bromopyridazin-3-amine (310 mg, 1.79 mmol), Pd(PPh₃)₄ (103 mg, 0.089 mmol), and potassium carbonate (740 mg, 5.36 mmol) were mixed in H₂O/Dimethylformamide (DMF) (4.3/1.3 mL) and heated in a microwave reactor for 35 minutes at 110° C. The reaction mixture was concentrated. The residue was purified by MPLC to give 6-(3-fluorophenyl)pyridazin-3-amine (255 mg, 75%) as a white solid.

¹H NMR (400 MHz, CDCl₃) δ 7.77-7.71 (m, 2H), 7.64 (d, J=9.3 Hz, 1H), 7.52-7.42 (m, 1H), 7.16-7.10 (m, 1H), 6.86 (d, J=9.2 Hz, 1H), 4.90 (s, 2H).

### Synthesis of [1,1′-biphenyl]-4-amine

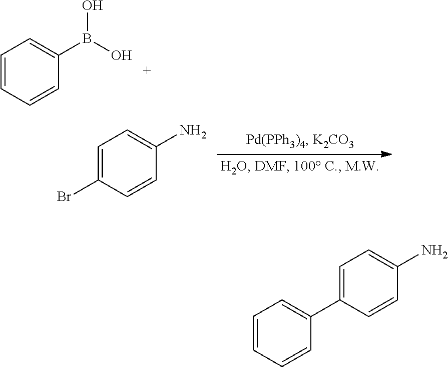 [SMILES: OB(O)c1ccccc1 ; Nc1ccc(Br)cc1 ; Nc1ccc(-c2ccccc2)cc1]

Phenylboronic acid (255 mg, 2.09 mmol), 4-bromoaniline (300 mg, 1.74 mmol), Pd(PPh₃)₄ (100 mg, 0.087 mmol), and potassium carbonate (891 mg, 6.45 mmol) were mixed in H₂O/DMF (3.5/3.5 mL) and heated in a microwave reactor for 30 minutes at 100° C. The reaction mixture was extracted by ethyl acetate (EA) and brine. The organic layer was dried over anhydrous Na₂SO₄ and concentrated. The residue was purified by MPLC to give [1,1′-biphenyl]-4-amine (257 mg, 87%) as a yellow solid.

¹H NMR (400 MHz, CDCl₃) δ 7.58-7.55 (m, 2H), 7.49-7.38 (m, 4H), 7.32-7.27 (m, 1H), 6.81-6.76 (m, 2H), 3.75 (s, 2H).

### Synthesis of 5-phenylpyrazin-2-amine

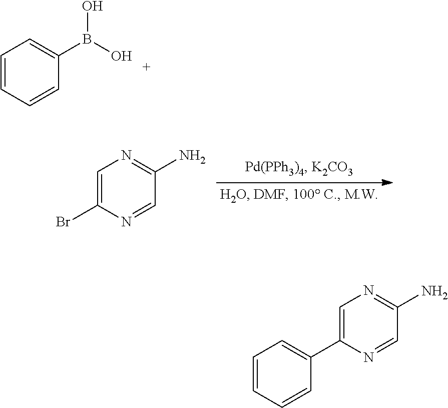

Phenylboronic acid (255 mg, 2.09 mmol), 5-bromopyrazin-2-amine (303 mg, 1.74 mmol), Pd(PPh₃)₄ (100 mg, 0.087 mmol), and potassium carbonate (891 mg, 6.45 mmol) were mixed in H₂O/DMF (3.5/3.5 mL) and heated in a microwave reactor for 30 minutes at 100° C. The reaction mixture was extracted by EA and brine. The organic layer was dried over anhydrous Na₂SO₄ and concentrated. The residue was purified by MPLC to give 5-phenylpyrazin-2-amine (240 mg, 81%) as a yellow solid.

¹H NMR (400 MHz, CDCl₃) δ 8.49 (s, 1H), 8.09 (d, J=1.4 Hz, 1H), 7.90 (d, J=7.4 Hz, 2H), 7.50-7.46 (m, 2H), 7.41-7.36 (m, 1H), 4.70-4.57 (m, 2H).

### Synthesis of 5-phenylpyridin-2-amine

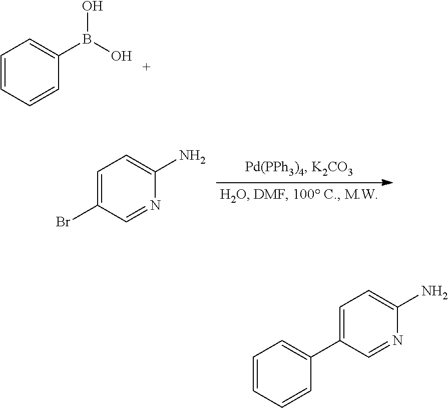

Phenylboronic acid (255 mg, 2.09 mmol), 6-bromopyridin-3-amine (300 mg, 1.74 mmol), Pd(PPh₃)₄ (100 mg, 0.087 mmol), and potassium carbonate (891 mg, 6.45 mmol) were mixed in H₂O/DMF (3.5/3.5 mL) and heated in a microwave reactor for 30 minutes at 100° C. The reaction mixture was extracted by EA and brine. The organic layer was dried over anhydrous Na₂SO₄ and concentrated. The residue was purified by MPLC to give 5-phenylpyridin-2-amine (251 mg, 84%) as an orange solid.

¹H NMR (400 MHz, CDCl₃) δ 8.35 (d, J=1.9 Hz, 1H), 7.70 (dd, J=2.4, 8.8 Hz, 1H), 7.56-7.51 (m, 2H), 7.45 (t, J=7.6 Hz, 2H), 7.34 (t, J=7.3 Hz, 1H), 6.61 (d, J=8.5 Hz, 1H), 4.57-4.46 (m, 2H).

### Synthesis of 5-(3-fluorophenyl)pyrimidin-2-amine

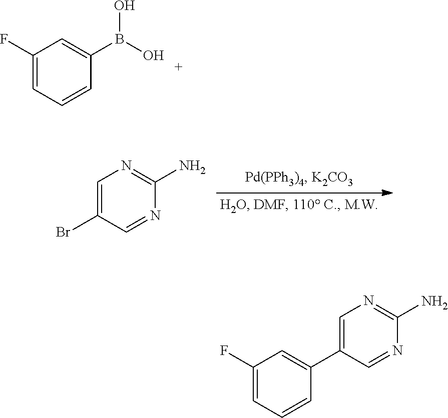

(3-Fluorophenyl)boronic acid (500 mg, 3.57 mmol), 5-bromopyrimidin-2-amine (518 mg, 2.98 mmol), Pd(PPh₃)₄ (172 mg, 0.15 mmol), and potassium carbonate (1.23 g, 8.93 mmol) were mixed in H₂O/DMF (6/6 mL) and heated in a microwave reactor for 35 minutes at 110° C. The reaction mixture was extracted by dichloromethane (DCM) and brine. The organic layer was dried over anhydrous Na₂SO₄ and concentrated. The crude mixture was solidified by using EA and n-hexane (HEX) to give 5-(3-fluorophenyl)pyrimidin-2-amine (271 mg, 48%) as a grey solid.

¹H NMR (400 MHz, CDCl₃) δ 8.55 (s, 2H), 7.54-7.32 (m, 1H), 7.31-7.23 (m, 1H), 7.23-7.18 (m, 1H), 7.11-7.05 (m, 1H), 5.25 (s, 2H).

### Synthesis of 6-phenylpyridazin-3-amine

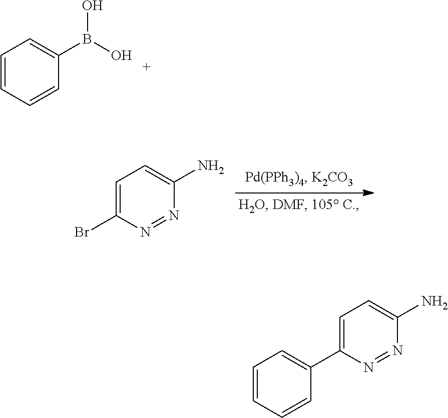

Phenylboronic acid (2.5 g, 20.7 mmol), 6-bromopyridazin-3-amine (3 g, 17.2 mmol), Pd(PPh₃)₄ (996 mg, 0.86 mmol), and potassium carbonate (8.3 g, 60.3 mmol) were mixed in H₂O/DMF (34/39 mL) and stirred for 21 hours at 105° C. The reaction mixture was extracted by EA and brine. The organic layer was dried over anhydrous Na₂SO₄ and concentrated. The residue was purified by MPLC to give 6-phenylpyridazin-3-amine (1.9 g, 63%) as a white solid.

¹H NMR (400 MHz, CDCl₃) δ 8.00-7.98 (m, 2H), 7.66 (d, J=9.1 Hz, 1H), 7.61-7.39 (m, 3H), 6.85 (d, J=9.3 Hz, 1H), 4.76 (s, 2H).

### Synthesis of 5-phenylpyrimidin-2-amine

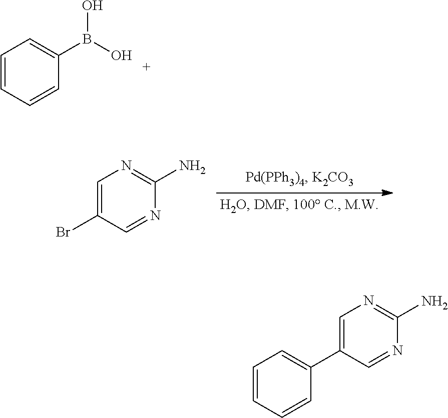

Phenylboronic acid (255 mg, 2.09 mmol), 5-bromopyrimidin-2-amine (303 mg, 1.74 mmol), Pd(PPh₃)₄ (100 mg, 0.087 mmol), and potassium carbonate (891 mg, 6.45 mmol) were mixed in H₂O/DMF (3.5/3.5 mL) and heated in a microwave reactor for 30 minutes at 100° C. The reaction mixture was extracted by EA and brine. The organic layer was dried over anhydrous Na₂SO₄ and concentrated. The residue was purified by MPLC to give 5-phenylpyrimidin-2-amine (275 mg, 92%) as a yellow solid.

¹H NMR (400 MHz, CDCl₃) δ 8.56 (s, 2H), 7.53-7.46 (m, 4H), 7.42-7.36 (m, 1H), 5.13 (d, J=1.8 Hz, 2H).

### Synthesis of 5-(furan-3-yl)pyrimidin-2-amine

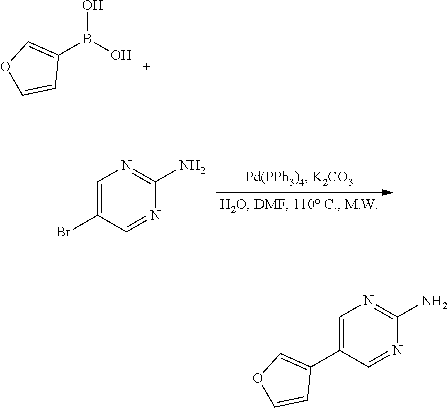

Furan-3-ylboronic acid (617 mg, 5.52 mmol), 5-bromopyrimidin-2-amine (800 mg, 4.6 mmol), Pd(PPh₃)₄ (266 mg, 0.23 mmol), and potassium carbonate (1.9 g, 13.8 mmol) were mixed in H₂O/DMF (9.2/9.2 mL) and heated in a microwave reactor for 35 minutes at 110° C. The reaction mixture was extracted by EA and brine. The organic layer was dried over anhydrous Na₂SO₄ and concentrated. The crude mixture was solidified by using EA and HEX to give 5-(furan-3-yl)pyrimidin-2-amine (403 mg, 54%) as a grey solid.

¹H NMR (400 MHz, DMSO-d₆) δ 8.52 (s, 2H), 8.11 (s, 1H), 7.73 (dd, J=1.7, 1.7 Hz, 1H), 6.93 (d, J=1.1 Hz, 1H), 6.71 (s, 2H).

### Synthesis of 5-(3-fluorophenyl)pyridin-2-amine

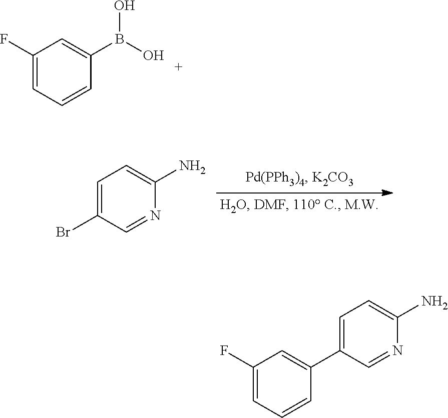

(3-Fluorophenyl)boronic acid (873 mg, 6.24 mmol), 5-bromopyridin-2-amine (900 mg, 5.2 mmol), Pd(PPh₃)₄ (301 mg, 0.26 mmol), and potassium carbonate (2.2 g, 15.6 mmol) were mixed in H₂O/DMF (10.4/10.4 mL) and heated in a microwave reactor for 60 minutes at 110° C. The reaction mixture was extracted by EA and brine. The organic layer was dried over anhydrous Na₂SO₄ and concentrated. The residue was purified by MPLC to give 5-(3-fluorophenyl)pyridin-2-amine (829 mg, 85%) as a white solid.

¹H NMR (400 MHz, DMSO-d₆) δ 8.30 (d, J=2.0 Hz, 1H), 7.74 (dd, J=2.6, 8.6 Hz, 1H), 7.66-7.53 (m, 1H), 7.45-7.41 (m, 2H), 7.11-7.05 (m, 1H), 6.53-6.50 (m, 1H), 6.17 (s, 2H).

2) Suzuki Coupling B

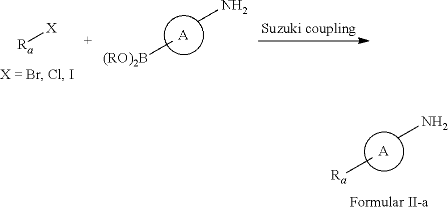

### Synthesis of 4-(pyridin-2-yl)aniline

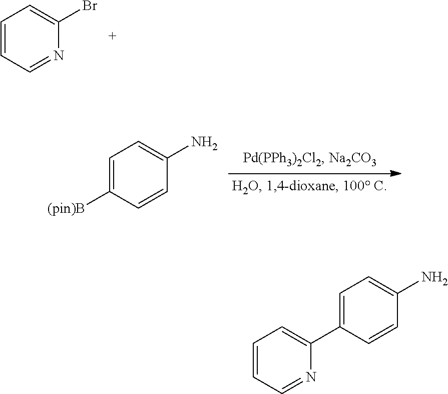

2-Bromopyridine (1.16 mL, 12 mmol), 4-(4,4,5,5-tetramethyl-1,3,2-dioxaborolan-2-yl)aniline (2.19 g, 10 mmol), Pd(PPh₃)₂Cl₂ (0.70 g, 1 mmol), and Na₂CO₃ (3.18 g, 30 mmol) were mixed in H₂O/1,4-dioxane (12.5/37.5 mL) and stirred for 21 hours at 100° C. The reaction mixture was concentrated and purified by MPLC to give 4-(pyridin-2-yl)aniline (1.57 g, 92%) as an orange solid.

¹H NMR (400 MHz, DMSO-d₆) δ 8.54-8.51 (m, 1H), 7.81-7.72 (m, 4H), 7.17-7.13 (m, 1H), 6.66-6.60 (m, 2H), 5.44 (s, 2H).

### Synthesis of 4-(pyridin-3-yl)aniline

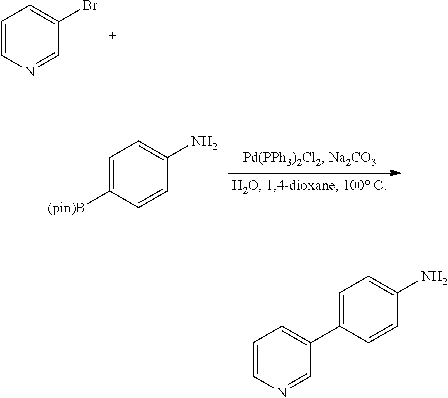

3-Bromopyridine (1.17 mL, 12 mmol), 4-(4,4,5,5-tetramethyl-1,3,2-dioxaborolan-2-yl)aniline (2.19 g, 10 mmol), Pd(PPh₃)₂Cl₂ (0.70 g, 1 mmol), and Na₂CO₃ (3.18 g, 30 mmol) were mixed in H₂O/1,4-dioxane (12.5/37.5 mL) and stirred for 16 hours at 100° C. The reaction mixture was concentrated and purified by MPLC to give 4-(pyridin-3-yl)aniline (1.57 g, 92%) as a pale yellow solid.

¹H NMR (400 MHz, DMSO-d₆) δ 8.78 (d, J=2.1 Hz, 1H), 8.41 (dd, J=1.4, 4.7 Hz, 1H), 7.94-7.90 (m, 1H), 7.66-7.53 (m, 1H), 7.45-7.40 (m, 2H), 6.70-6.64 (m, 2H), 5.34 (s, 2H).

### Synthesis of 4-(pyridin-4-yl)aniline

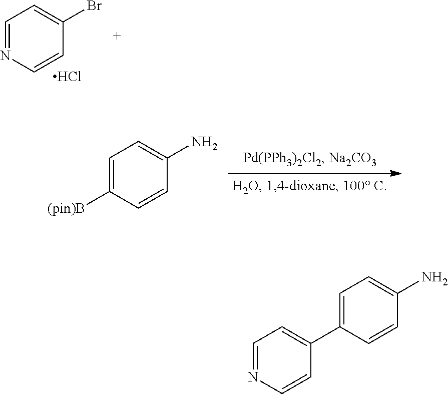

4-Bromopyridine hydrochloride (2.33 g, 12 mmol), 4-(4,4,5,5-tetramethyl-1,3,2-dioxaborolan-2-yl)aniline (2.19 g, 10 mmol), Pd(PPh₃)₂Cl₂ (0.70 g, 1 mmol), and Na₂CO₃ (3.18 g, 30 mmol) were mixed in H₂O/1,4-dioxane (12.5/37.5 mL) and stirred for 16 hours at 100° C. The reaction mixture was concentrated and purified by MPLC to give crude 4-(pyridin-4-yl)aniline (1.16 g, 67%) as a pale brown solid.

¹H NMR (400 MHz, DMSO-d₆) δ 8.49-8.47 (m, 2H), 7.58-7.52 (m, 4H), 6.69-6.64 (m, 2H), 5.53 (s, 2H).

### Synthesis of 4-(pyrimidin-2-yl)aniline

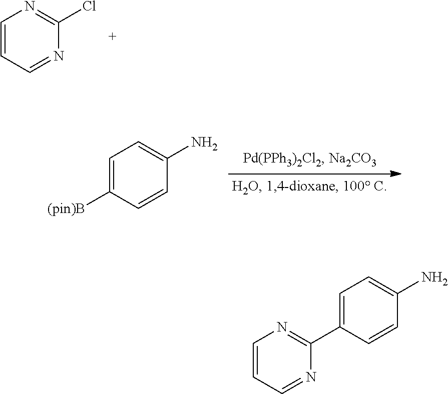

2-Chloropyrimidine (1.37 g, 12 mmol), 4-(4,4,5,5-tetramethyl-1,3,2-dioxaborolan-2-yl)aniline (2.19 g, 10 mmol), Pd(PPh₃)₂Cl₂ (0.70 g, 1 mmol), and Na₂CO₃ (3.18 g, 30 mmol) were mixed in H₂O/1,4-dioxane (12.5/37.5 mL) and stirred for 16 hours at 100° C. The reaction mixture was concentrated and purified by MPLC to give 4-(pyrimidin-2-yl)aniline (1.37 g, 80%) as a brown solid.

¹H NMR (400 MHz, DMSO-d₆) δ 8.73 (d, J=4.9 Hz, 2H), 8.12-8.07 (m, 2H), 7.21 (t, J=4.8 Hz, 1H), 6.65-6.60 (m, 2H), 5.68 (s, 2H).

### Synthesis of 4-(pyrazin-2-yl)aniline

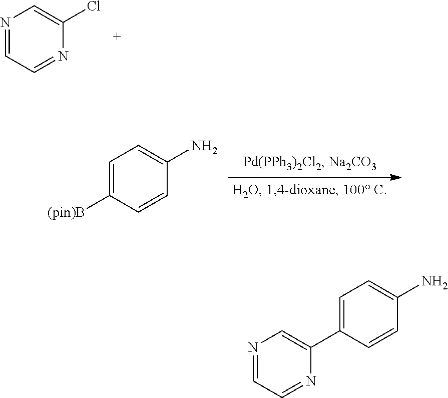

2-Chloropyrazine (1.07 mL, 12 mmol), 4-(4,4,5,5-tetramethyl-1,3,2-dioxaborolan-2-yl)aniline (2.19 g, 10 mmol), Pd(PPh₃)₂Cl₂ (0.70 g, 1 mmol), and Na₂CO₃ (3.18 g, 30 mmol) were mixed in H₂O/1,4-dioxane (12.5/37.5 mL) and stirred for 16 hours at 100° C. The reaction mixture was concentrated and purified by MPLC to give 4-(pyrazin-2-yl)aniline (1.35 g, 78%) as a brown solid.

¹H NMR (400 MHz, DMSO-d₆) δ 9.06 (d, J=1.5 Hz, 1H), 8.55-8.54 (m, 1H), 8.38 (d, J=2.5 Hz, 1H), 7.89-7.83 (m, 2H), 6.69-6.64 (m, 2H), 5.63 (s, 2H).

### Synthesis of 4-(pyrimidin-5-yl)aniline

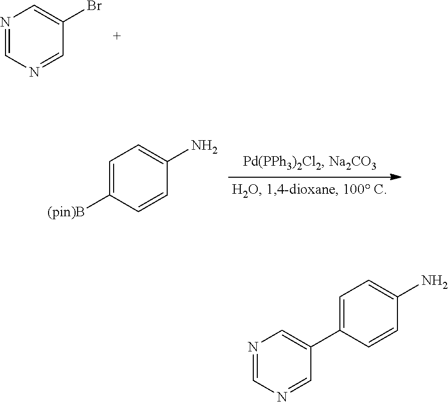

5-Bromopyrimidine (1.91 g, 12 mmol), 4-(4,4,5,5-tetramethyl-1,3,2-dioxaborolan-2-yl)aniline (2.19 g, 10 mmol), Pd(PPh₃)₂Cl₂ (0.70 g, 1 mmol) and Na₂CO₃ (3.18 g, 30 mmol) were combined in H₂O/1,4-dioxane (12.5/37.5 mL) and stirred for 16 hours at 100° C. The reaction mixture was concentrated and purified by MPLC to give crude 4-(pyrimidin-5-yl)aniline (1.50 g, 87%) as a pale brown solid.

¹H NMR (400 MHz, DMSO-d₆) δ 9.02 (s, 1H), 9.01 (s, 2H), 7.53-7.48 (m, 2H), 6.71-6.66 (m, 2H), 5.49 (s, 2H).

### Synthesis of 4-(pyrimidin-4-yl)aniline

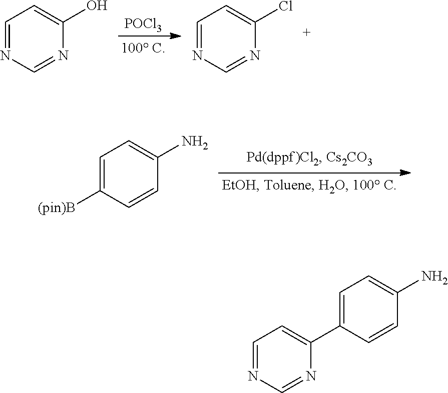

Step 1: 1H-Pyrimidin-6-one (10 g, 104 mmol) and POCl₃ (100 mL, 1.08 mol) were charged to a pressure flask. Flask was flushed with nitrogen and heated for 6 hours at 100° C. The reaction mixture was concentrated under reduced pressure to remove POCl₃. The reaction mixture was poured into EA carefully and stirred for 30 minutes. The reaction mixture was filtered, and the filter cake was washed with ethyl acetate, dried to give 4-chloropyrimidine (3.50 g, crude) as a brown solid.

¹H NMR (400 MHz, DMSO-d₆) δ 9.14 (s, 1H), 8.07 (d, J=7.20 Hz, 1H), 6.62 (d, J=7.60 Hz, 1H).

Step 2: A mixture of 4-chloropyrimidine (1.80 g, 15.7 mmol), 4-(4,4,5,5-tetramethyl-1,3,2-dioxaborolan-2-yl)aniline (3.79 g, 17.3 mmol), Cs₂CO₃ (20.5 g, 62.9 mmol), 1,1′-bis(diphenylphosphino)ferrocene]dichloropalladium(II) (575 mg, 0.79 mmol) in toluene (12 mL), ethanol (4 mL), and H₂O (3.6 mL) and the mixture was degassed and purged with N₂ for 3 times, and then the mixture was stirred for 12 hours at 100° C. under N₂ atmosphere. Thin-layer chromatography (TLC) indicated 4-chloropyrimidine was consumed completely, and one major new spot with larger polarity was detected. The reaction mixture was diluted with water and extracted with EA. The combined organic layers were washed with brine, dried over Na₂SO₄, filtered, and concentrated under reduced pressure to give a residue. The reaction mixture was concentrated and purified by column chromatography to give 4-(pyrimidin-4-yl)aniline (1.70 g, crude) as a yellow solid.

¹H NMR (400 MHz, DMSO-d₆) δ 9.02 (s, 1H), 8.62 (d, J=5.20 Hz, 1H), 7.94 (d, J=8.80 Hz, 2H), 7.79-7.83 (m, 1H), 6.65 (d, J=8.80 Hz, 2H), 5.80 (s, 2H)

2. Synthesis of Formula II-b

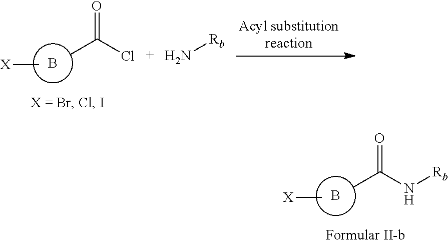

### Synthesis of 3-bromo-N-((5-methylfuran-2-yl)methyl)benzamide

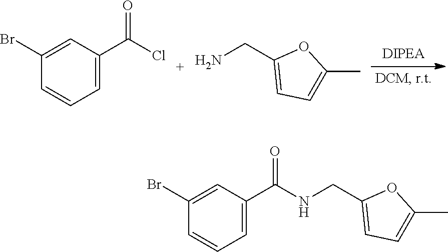

3-Bromobenzoyl chloride (2.17 mL, 16.5 mmol) and 1-(5-methylfuran-2-yl)methanamine (1.5 mL, 13.7 mmol) were dissolved in DCM (137 mL), followed up by addition of N,N-diisopropylethylamine (DIPEA) (5.14 mL, 29.6 mmol) and stirred for 18 hours at room temperature (r.t.). The reaction mixture was extracted by DCM and saturated aqueous (aq.) NaHCO₃. The organic layer was dried over anhydrous Na₂SO₄ and concentrated. The residue was purified by MPLC to give 3-bromo-N-((5-methylfuran-2-yl)methyl)benzamide (4 g, >99%) as an orange oil.

¹H NMR (400 MHz, CDCl₃) δ 7.95 (dd, J=1.8, 1.8 Hz, 1H), 7.74-7.71 (m, 1H), 7.65 (ddd, J=1.0, 2.0, 8.0 Hz, 1H), 7.33 (dd, J=7.9, 7.9 Hz, 1H), 6.31 (s, 1H), 6.20 (d, J=3.0 Hz, 1H), 5.94 (dd, J=1.0, 3.0 Hz, 1H), 4.59 (d, J=5.3 Hz, 2H), 2.31 (s, 3H).

### Synthesis of 3-bromo-N-phenethylbenzamide

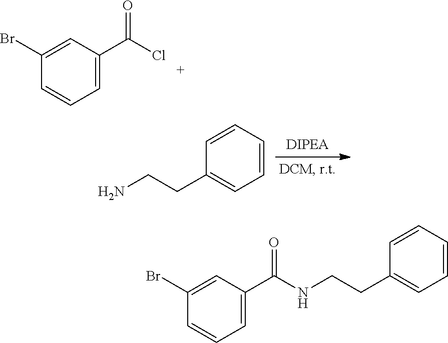 [SMILES: O=C(Cl)c1cccc(Br)c1 ; NCCc1ccccc1 ; O=C(NCCc1ccccc1)c1cccc(Br)c1]

3-Bromobenzoyl chloride (1.3 mL, 9.9 mmol) and 2-phenylethan-1-amine (1 mL, 8.25 mmol) were dissolved in DCM (82 mL), followed up by addition of DIPEA (3 mL, 17.7 mmol) and stirred for 25 hours at room temperature. The reaction mixture was extracted by DCM and saturated aq. NH₄Cl. The organic layer was dried over anhydrous Na₂SO₄ and concentrated. The crude mixture was solidified by using EA and HEX to give 3-bromo-N-phenethylbenzamide (1.8 g, 73%) as a yellowish white solid.

¹H NMR (400 MHz, DMSO-d₆) δ 8.71 (t, J=5.4 Hz, 1H), 7.99 (dd, J=1.8, 1.8 Hz, 1H), 7.82 (dd, J=1.3, 6.4 Hz, 1H), 7.75-7.72 (m, 1H), 7.44 (dd, J=7.9, 7.9 Hz, 1H), 7.33-7.28 (m, 2H), 7.26-7.19 (m, 3H), 3.51-3.45 (m, 2H), 2.85 (t, J=7.4 Hz, 2H).

### Synthesis of 3-bromo-N-(3-phenylpropyl)benzamide

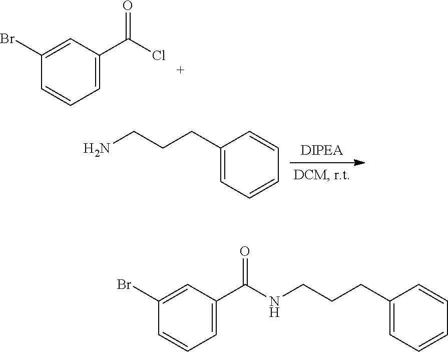 [SMILES: O=C(Cl)c1cccc(Br)c1 ; NCCCc1ccccc1 ; O=C(NCCCc1ccccc1)c1cccc(Br)c1]

3-Bromobenzoyl chloride (0.82 mL, 6.2 mmol) and 3-phenylpropan-1-amine (0.74 mL, 5.18 mmol) were dissolved in DCM (52 mL), followed up by addition of DIPEA (1.9 mL, 11 mmol) and stirred for 25 hours at room temperature. The reaction mixture was extracted by DCM and saturated aq. NH₄Cl. The organic layer was dried over anhydrous Na₂SO₄ and concentrated to give 3-bromo-N-(3-phenylpropyl)benzamide (2.17 g, >99%) as a brown oil.

¹H NMR (400 MHz, DMSO-d₆) δ 8.66-8.60 (m, 1H), 8.03-8.01 (m, 1H), 7.86-7.84 (m, 1H), 7.75-7.72 (m, 1H), 7.46-7.42 (m, 1H), 7.31-7.26 (m, 2H), 7.24-7.18 (m, 3H), 3.28 (dd, J=6.9, 12.8 Hz, 2H), 2.66-2.60 (m, 2H), 1.88-1.79 (m, 2H).

### Synthesis of 3-bromo-N-(2-cyclohexylethyl)benzamide

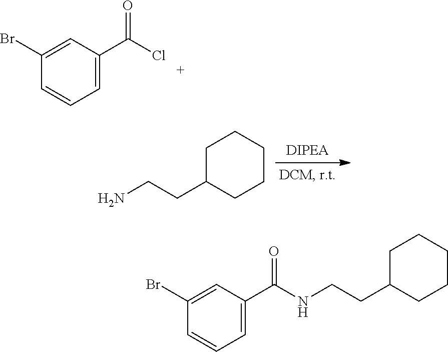 [SMILES: O=C(Cl)c1cccc(Br)c1 ; NCCC1CCCCC1 ; O=C(NCCC1CCCCC1)c1cccc(Br)c1]

3-Bromobenzoyl chloride (0.12 mL, 0.9 mmol) and 2-cyclohexylethan-1-amine (0.13 mL, 0.9 mmol) were dissolved in DCM (9.1 mL), followed up by addition of DIPEA (0.34 mL, 1.9 mmol) and stirred for 23 hours at room temperature. The reaction mixture was extracted by DCM and saturated aq. NH₄Cl. The organic layer was dried over anhydrous Na₂SO₄ to give crude 3-bromo-N-(2-cyclohexylethyl)benzamide (300 mg, >99%) as a yellow solid.

¹H NMR (400 MHz, DMSO-d₆) δ 8.54 (t, J=5.3 Hz, 1H), 8.01 (t, J=1.8 Hz, 1H), 7.85-7.83 (m, 1H), 7.74-7.71 (m, 1H), 7.43 (t, J=7.9 Hz, 1H), 3.32-3.24 (m, 2H), 1.76-0.87 (m, 13H).

### Synthesis of 3-bromo-N-((1R,2S)-2-phenylcyclopropyl)benzamide

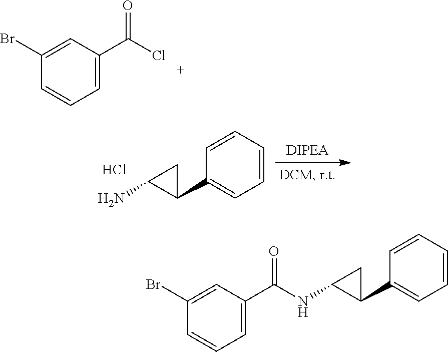 [SMILES: O=C(Cl)c1cccc(Br)c1 ; Cl ; N[C@@H]1C[C@H]1c1ccccc1 ; O=C(N[C@@H]1C[C@H]1c1ccccc1)c1cccc(Br)c1]

3-Bromobenzoyl chloride (0.2 mL, 0.9 mmol) and (1R,2S)-2-phenylcyclopropan-1-amine hydrochloride (129 mg, 0.76 mmol) were dissolved in DCM (7.6 mL), followed up by addition of DIPEA (0.42 mL, 2.4 mmol) and stirred for 20 hours at room temperature. The reaction mixture was extracted by 10% methanol (MeOH) in DCM and saturated aq. NH₄Cl. The organic layer was dried over anhydrous Na₂SO₄ to give crude 3-bromo-N-((1R,2S)-2-phenylcyclopropyl)benzamide (297 mg, >99%) as a beige solid.

¹H NMR (400 MHz, DMSO-d₆) δ 8.81 (d, J=4.3 Hz, 1H), 8.03 (t, J=1.8 Hz, 1H), 7.86-7.84 (m, 1H), 7.76-7.73 (m, 1H), 7.45 (t, J=7.9 Hz, 1H), 7.29 (t, J=7.4 Hz, 2H), 7.20-7.14 (m, 3H), 3.07-3.00 (m, 1H), 2.13-2.07 (m, 1H), 1.39-1.33 (m, 1H), 1.28-1.17 (m, 1H).

3. Synthesis of Formula II-c

1) Buchwald-Hartwig Coupling

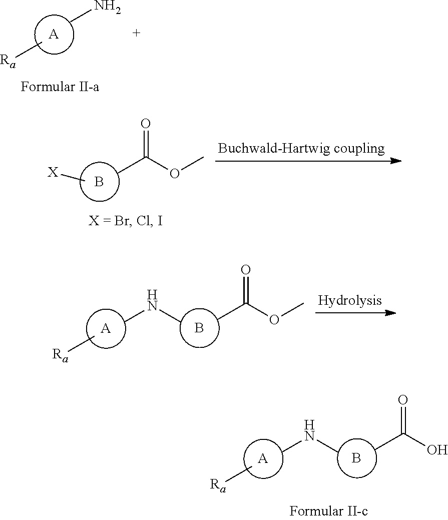

### Synthesis of 3-((6-phenylpyridazin-3-yl)amino)benzoic acid

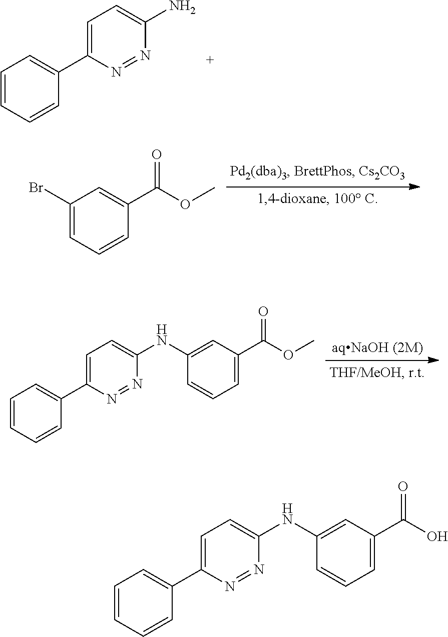 [SMILES: Nc1ccc(-c2ccccc2)nn1 ; "+" ; COC(=O)c1cccc(Br)c1 ; COC(=O)c1cccc(Nc2ccc(-c3ccccc3)nn2)c1 ; O=C(O)c1cccc(Nc2ccc(-c3ccccc3)nn2)c1]

Step 1: To a solution of methyl 3-bromobenzoate (18 g, 83.7 mmol) in 1,4-dioxane (90 mL) was added 6-phenylpyridazin-3-amine (15.1 g, 87.9 mmol), BrettPhos (8.99 g, 16.7 mmol), and cesium carbonate (68.2 g, 209 mmol). Pd₂(dba)₃ (2.3 g, 2.51 mmol) was added into the solution. The solution was stirred for 6 hours at 100° C. The reaction was filtered, and the filter cake was triturated with tetrahydrofuran (THF) (180 mL) and MeOH (35 mL) for 2 hours at room temperature. Then the suspension was filtered, and filtrate was concentrated under reduced pressure to give a residue. The residue was dissolved in THF (200 mL). The solution was filtered through a pad of silica gel. The filtrate was concentrated under vacuum to give methyl 3-[(6-phenylpyridazin-3-yl)amino]benzoate (10.2 g, 40%) as a yellow solid.

¹H NMR (400 MHz, DMSO-d₆) δ 9.93 (s, 1H), 8.48 (s, 1H), 8.15 (d, J=6.0 Hz, 1H), 8.05 (t, J=5.6 Hz, 3H), 7.61 (d, J=7.6 Hz, 1H), 7.53 (m, J=6.0 Hz, 4H), 7.38 (d, J=9.2 Hz, 1H), 3.89 (s, 3H).

Step 2: Methyl 3-[(6-phenylpyridazin-3-yl)amino]benzoate (9 g, 29.5 mmol) was dissolved in MeOH/THF (7/45 mL). aq. NaOH (2 M, 29.4 mL) was added into the solution. The solution was stirred for 12 hours at room temperature. The reaction mixture was concentrated under reduced pressure to remove MeOH and THF to give a residue. The H₂O (80 mL) was added into the residue. The pH value of the suspension was adjusted to 2 by aq. HCl (2 M). THF (30 mL) was added into the suspension. The suspension was filtered, and the filter cake was dried under vacuum to give 3-((6-phenylpyridazin-3-yl)amino)benzoic acid (5 g, 58%) as yellow solid.

¹H NMR (400 MHz, DMSO-d₆) δ 12.93 (s, 1H), 9.61 (s, 1H), 8.50 (s, 1H), 8.06 (t, J=10.4 Hz, 4H), 7.57-7.46 (m, 5H), 7.24 (d, J=9.2 Hz, 1H).

### Synthesis of 3-((5-phenylpyrimidin-2-yl)amino)benzoic acid

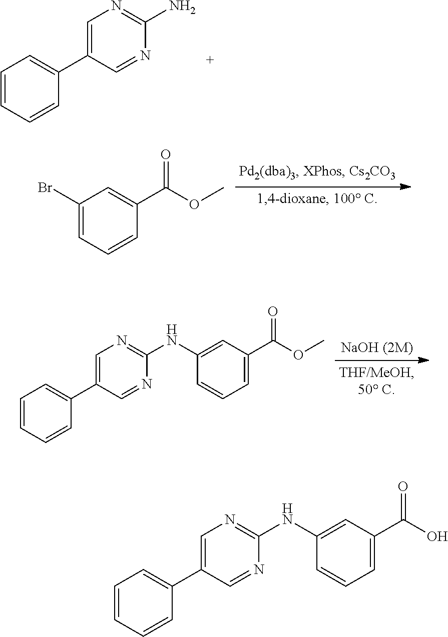 [SMILES: Nc1ncc(-c2ccccc2)cn1 ; "+" ; COC(=O)c1cccc(Br)c1 ; COC(=O)c1cccc(Nc2ncc(-c3ccccc3)cn2)c1 ; O=C(O)c1cccc(Nc2ncc(-c3ccccc3)cn2)c1]

Step 1: To a solution of 5-phenylpyrimidin-2-amine (22 g, 128 mmol) in 1,4-dioxane (130 mL) were added methyl 3-bromobenzoate (18.4 g, 85.7 mmol), cesium carbonate (83.7 g, 257 mmol), and XPhos (12.3 g, 25.7 mmol). Then Pd₂(dba)₃ (2.35 g, 2.57 mmol) was added into the solution. Then solution was stirred for 12 hours at 100° C. The reaction solution was poured into H₂O (500 mL). The suspension was filtered, and the filter cake was rinsed with H₂O (100 mL). The filter cake was dried in vacuum to give the crude product. The crude product was diluted with THF (1 L). The resulting suspension was filtered, and the filter cake was washed with THF (200 mL). The filtrate was purified by column chromatography to give methyl 3-[(5-phenylpyrimidin-2-yl)amino]benzoate (9 g, 34%) as a white solid.

¹H NMR (400 MHz, DMSO-d₆) δ 10.03 (s, 1H), 8.88 (s, 2H), 8.05 (d, J=1.2 Hz, 1H), 8.05 (t, J=1.6 Hz, 1H), 7.75 (d, J=8.4 Hz, 2H), 7.57-7.38 (m, 5H), 3.86 (s, 3H).

Step 2: An aq. NaOH (2 M, 29.5 mL) was added into a solution of methyl 3-[(5-phenylpyrimidin-2-yl)amino]benzoate (9 g, 29.5 mmol) in THF (70 mL). Then MeOH (50 mL) was added into the reaction solution. The solution was stirred for 12 hours at 50° C. The reaction solution was concentrated to give a crude product. The crude product was added into H₂O (500 mL). Then pH value of the solution was adjusted to 1-2 by aq. HCl (1 M). The suspension was filtered, and the filter cake was washed with H₂O (200 mL). The filter cake was dried under vacuum to give 3-((5-phenylpyrimidin-2-yl)amino)benzoic acid (5 g, 58%) as white solid.

¹H NMR (400 MHz, DMSO-d₆) δ 12.89 (s, 1H), 9.99 (s, 1H), 8.88 (s, 2H), 8.46 (d, J=1.6 Hz, 1H), 8.02 (d, J=1.2 Hz, 1H), 7.74 (d, J=8.4 Hz, 2H), 7.55-7.36 (m, 5H).

### Synthesis of 3-((5-(3-fluorophenyl)pyridin-2-yl)amino)benzoic acid

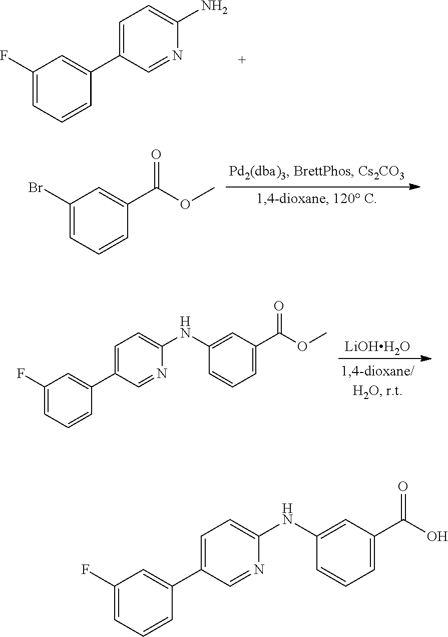

Step 1: 5-(3-Fluorophenyl)pyridin-2-amine (700 mg, 3.72 mmol), methyl 3-bromobenzoate (1.2 g, 4.84 mmol), Pd₂(dba)₃ (340 mg, 0.37 mmol), BrettPhos (339 mg, 0.74 mmol), and cesium carbonate (2.4 g, 7.44 mmol) were mixed in 1,4-dioxane (18.6 mL) and heated in a microwave reactor for 90 minutes at 120° C. The reaction mixture was extracted by EA and brine. The organic layer was dried over anhydrous Na₂SO₄ and concentrated. The crude mixture was solidified by using DCM and HEX to give methyl 3-{[5-(3-fluorophenyl)pyridin-2-yl]amino}benzoate (539 mg, 45%) as a beige solid.

¹H NMR (400 MHz, DMSO-d₆) δ 9.52 (s, 1H), 8.60 (d, J=2.3 Hz, 1H), 8.36 (dd, J=1.9, 1.9 Hz, 1H), 8.07 (ddd, J=1.1, 2.3, 8.1 Hz, 1H), 7.99 (dd, J=2.6, 8.8 Hz, 1H), 7.61-7.40 (m, 5H), 7.19-7.13 (m, 1H), 6.94 (d, J=8.8 Hz, 1H), 3.87 (s, 3H).

Step 2: Methyl 3-{[5-(3-fluorophenyl)pyridin-2-yl]amino}benzoate (400 mg, 1.24 mmol) and LiOH.H₂O (521 mg, 12.4 mmol) were mixed in H₂O/1,4-dioxane (5.2/24.8 mL) and stirred for 18 hours at room temperature. The reaction mixture acidified by adding 1 N HCl and extracted by EA and brine. The organic layer was dried over anhydrous Na₂SO₄ and concentrated. The crude mixture was solidified by using EA to give 3-((5-(3-fluorophenyl)pyridin-2-yl)amino)benzoic acid (348 mg, 91%) as a beige solid.

¹H NMR (400 MHz, DMSO-d₆) δ 12.90 (s, 1H), 9.47 (s, 1H), 8.60 (d, J=2.3 Hz, 1H), 8.35 (dd, J=1.9, 1.9 Hz, 1H), 8.10-7.95 (m, 2H), 7.61-7.31 (m, 5H), 7.18-7.12 (m, 1H), 6.94 (d, J=8.5 Hz, 1H).

### Synthesis of 3-((5-(3-fluorophenyl)pyrimidin-2-yl)amino)benzoic acid

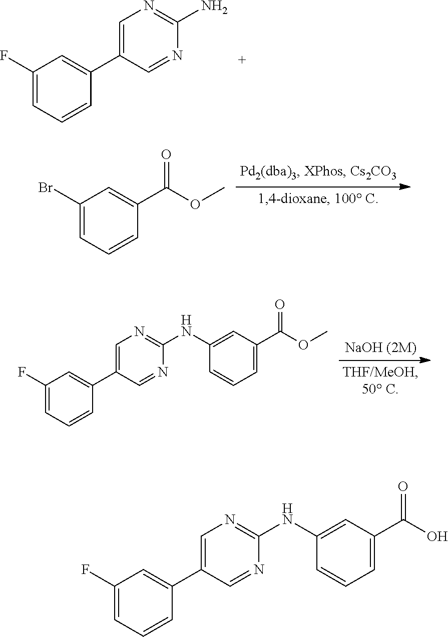 [SMILES: Nc1ncc(-c2cccc(F)c2)cn1 ; "+" ; COC(=O)c1cccc(Br)c1 ; COC(=O)c1cccc(Nc2ncc(-c3cccc(F)c3)cn2)c1 ; O=C(O)c1cccc(Nc2ncc(-c3cccc(F)c3)cn2)c1]

Step 1: To a solution of 5-(3-fluorophenyl)pyrimidin-2-amine (30 g, 158 mmol) in 1,4-dioxane (210 mL) was methyl 3-bromobenzoate (31 g, 144 mmol), XPhos (20.6 g, 43.3 mmol), and cesium carbonate (141 g, 432 mmol). Then Pd₂(dba)₃ (3.96 g, 4.32 mmol) was added into the solution. The solution was stirred for 12 hours at 100° C. The reaction solution was poured into H₂O (500 mL), and the suspension was filtered. The filter cake was washed with H₂O (100 mL) and dried under vacuum to give a crude product. The crude product was added into THF (1 L). The suspension was filtered, and the filter cake was washed with THF (200 mL). The filtrate was purified by column chromatography to give methyl 3-{[5-(3-fluorophenyl)pyrimidin-2-yl]amino}benzoate (10 g, 22%) as a white solid.

¹H NMR (400 MHz, DMSO-d₆) δ 10.10 (s, 1H), 8.93 (s, 2H), 8.48 (d, J=2 Hz, 1H), 8.05 (d, J=8 Hz, 1H), 7.65-7.43 (m, 5H), 7.20 (m, 1H), 3.86 (s, 3H).

Step 2: An aq. NaOH (2 M, 30.9 mL) was added into a solution of methyl 3-{[5-(3-fluorophenyl)pyrimidin-2-yl]amino}benzoate (10 g, 30.9 mmol) in THF (70 mL). Then MeOH (50 mL) was added into the reaction solution. The solution was stirred for 12 hours at 50° C. The reaction solution was concentrated to give a crude product. The crude product was added into H₂O (500 mL). The pH value of the solution was adjusted to 1-2 by aq. HCl (1 M). The suspension was filtered. The filter cake was washed with H₂O (200 mL) and dried under vacuum to give 3-((5-(3-fluorophenyl)pyrimidin-2-yl)amino)benzoic acid (5 g, 52%) as a white solid.

¹H NMR (400 MHz, DMSO-d₆) δ 12.90 (s, 1H), 10.06 (s, 1H), 8.92 (s, 2H), 8.46 (s, 1H), 8.00 (d, J=8.0 Hz, 1H), 7.66-7.50 (m, 4H), 7.42 (t, J=9.2 Hz, 1H), 7.22-7.17 (m, 1H).

### Synthesis of 3-((5-phenylpyridin-2-yl)amino)benzoic acid

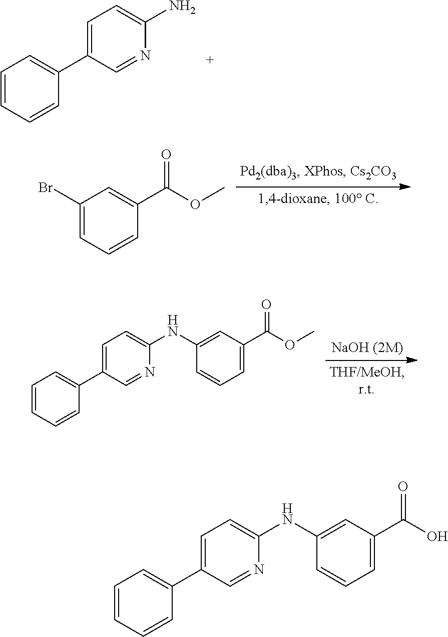 [SMILES: Nc1ccc(-c2ccccc2)cn1 ; "+" ; COC(=O)c1cccc(Br)c1 ; COC(=O)c1cccc(Nc2ccc(-c3ccccc3)cn2)c1 ; O=C(O)c1cccc(Nc2ccc(-c3ccccc3)cn2)c1]

Step 1: To a solution of methyl 3-bromobenzoate (20.8 g, 122 mmol) in 1,4-dioxane (125 mL) was added 5-phenylpyridin-2-amine (25.0 g, 116 mmol), XPhos (16.6 g, 34.8 mmol) and Cs₂CO₃ (113 g, 348 mmol). The solution was degassed and purged with N₂ for three times. Pd₂(dba)₃ (3.19 g, 3.49 mmol) was added into the solution. The solution was degassed and purged with N₂ for three times. The solution was stirred for 12 h at 100° C. The mixture suspension was filtered, and the filter cake was rinsed with EA. The filtrate was dried over sodium sulfate and filtered, concentrated under reduced pressure to give a residue. The residue was triturated with methyl tert-butyl ether (MTBE) for 1 hour at room temperature. The suspension was filtered, and the filter cake was rinsed with MTBE, and the filter cake was collected and dried under reduced pressure to give methyl 3-((5-phenylpyridin-2-yl)amino)benzoate (20.0 g, 56.5%) as a white solid.

¹H NMR (400 MHz, DMSO-d₆) δ 9.44 (s, 1H), 8.53 (d, J=2.0 Hz, 1H), 8.34 (s, 1H), 8.07 (d, J=8.4 Hz, 1H), 7.94 (dd, J=8.8, 2.4 Hz, 1H), 7.66 (d, J=7.6 Hz, 2H), 7.49-7.40 (m, 4H), 7.33 (t, J=7.6 Hz, 1H), 6.94 (d, J=8.8 Hz, 1H), 3.86 (s, 3H).

Step 2: Methyl 3-((5-phenylpyridin-2-yl)amino)benzoate (20.0 g, 65.7 mmol) was dissolved in MeOH (100 mL) and THF (20 mL). aq. NaOH (2 M, 65.7 mL) was added into the solution. The solution was stirred for 12 hours at room temperature. The reaction mixture was concentrated under reduced pressure to remove MeOH and THF to give a residue. The H₂O (80 mL) was added into the residue. The pH value of the suspension was adjusted to 5 by aq. HCl (6 M). The suspension was filtered, and the filter cake was concentrated under reduced pressure to give 3-((5-phenylpyridin-2-yl)amino)benzoic acid (10 g, 52%) as white solid.

¹H NMR (400 MHz, DMSO-d₆) δ 12.85 (s, 1H), 9.40 (s, 1H), 8.53 (d, J=2.4 Hz, 1H), 8.33-8.32 (m, 1H), 8.02-8.00 (m, 1H), 7.93 (dd, J=8.0, 2.4 Hz, 1H), 7.66 (d, J=7.2 Hz, 2H), 7.48-7.31 (m, 5H), 6.94 (d, J=8.8 Hz, 1H).

### Synthesis of 3-((5-(furan-3-yl)pyrimidin-2-yl)amino)benzoic acid

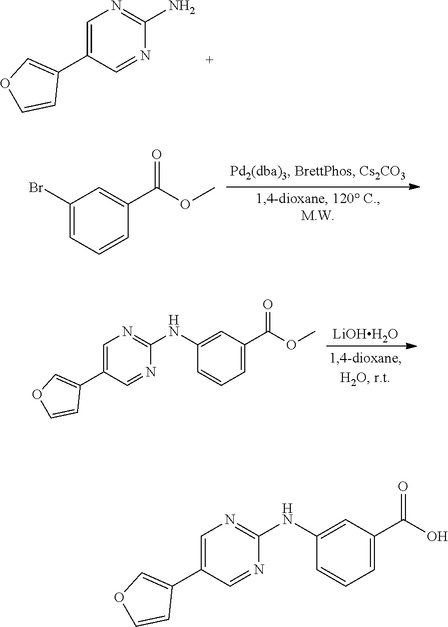

Step 1: 5-(Furan-3-yl)pyrimidin-2-amine (400 mg, 2.48 mmol), methyl 3-bromobenzoate (807 mg, 3.23 mmol), Pd₂(dba)₃ (227 mg, 0.25 mmol), BrettPhos (267 mg, 0.5 mmol), and cesium carbonate (1.6 g, 4.96 mmol) were mixed in 1,4-dioxane (12 mL) and heated in a microwave reactor for 90 minutes at 120° C. The reaction mixture was extracted by EA and brine. The crude mixture was solidified by using EA and HEX to give methyl 3-((5-(furan-3-yl)pyrimidin-2-yl)amino)benzoate (314 mg, 43%) as an orange solid.

¹H NMR (400 MHz, DMSO) δ 10.01 (s, 1H), 8.84 (s, 2H), 8.51 (dd, J=1.9, 1.9 Hz, 1H), 8.25 (s, 1H), 8.02-7.99 (m, 1H), 7.80 (dd, J=1.7, 1.7 Hz, 1H), 7.55 (d, J=7.8 Hz, 1H), 7.44 (dd, J=7.9, 7.9 Hz, 1H), 7.06 (d, J=1.0 Hz, 1H), 3.87-3.86 (m, 3H).

Step 2: Methyl 3-((5-(furan-3-yl)pyrimidin-2-yl)amino)benzoate (300 mg, 1.02 mmol) and LiOH.H₂O (426 mg, 10.2 mmol) were mixed in H₂O/1,4-dioxane (4.2/20.3 mL) and stirred for 18 hours at room temperature. Then pH value of the solution was adjusted to 1-2 by 1 N HCl. The reaction mixture was extracted by EA and brine. The crude mixture was solidified by using EA to give 3-((5-(furan-3-yl)pyrimidin-2-yl)amino)benzoic acid (199 mg, 70%) as a white solid.

¹H NMR (400 MHz, DMSO) δ 12.91 (s, 1H), 9.97 (s, 1H), 8.84 (s, 2H), 8.50 (dd, J=1.8, 1.8 Hz, 1H), 8.25 (s, 1H), 7.96 (dd, J=1.3, 8.1 Hz, 1H), 7.80 (dd, J=1.7, 1.7 Hz, 1H), 7.54 (d, J=7.8 Hz, 1H), 7.41 (dd, J=7.9, 7.9 Hz, 1H), 7.06 (d, J=1.0 Hz, 1H).

### Synthesis of 3-((4-(pyridin-2-yl)phenyl)amino)benzoic acid

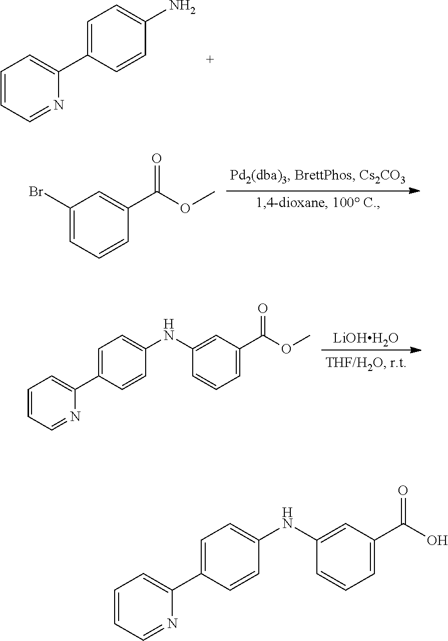

Step 1: To a solution of methyl 3-bromobenzoate (2.88 g, 13.4 mmol) in 1,4-dioxane (45 mL) was added 4-(pyridin-2-yl)aniline (1.52 g, 8.93 mmol), BrettPhos (0.96 g, 1.79 mmol), and cesium carbonate (11.64 g, 35.7 mmol). Pd₂(dba)₃ (0.82 g, 0.89 mmol) was added into the solution. The solution was stirred for 15 hours at 100° C. The reaction mixture was concentrated and purified by MPLC to give methyl 3-((4-(pyridin-2-yl)phenyl)amino)benzoate (1.36 g, 49%) as a yellow solid.

¹H NMR (400 MHz, DMSO-d₆) δ 8.73-8.71 (m, 1H), 8.61-8.59 (m, 1H), 8.05-8.01 (m, 2H), 7.91-7.87 (m, 1H), 7.86-7.80 (m, 1H), 7.74-7.71 (m, 1H), 7.46-7.40 (m, 3H), 7.28-7.24 (m, 1H), 7.21-7.17 (m, 2H), 3.85 (s, 3H).

Step 2: Methyl 3-((4-(pyridin-2-yl)phenyl)amino)benzoate (1.35 g, 4.43 mmol) and LiOH.H₂O (0.75 g, 17.73 mmol) were mixed in THF/H₂O (30/15 mL) and stirred for 117 hours at room temperature. The reaction mixture was extracted by EA and aq. HCl (1N). The organic layer was dried over anhydrous MgSO₄ and concentrated. The residue was purified by MPLC to give 3-((4-(pyridin-2-yl)phenyl)amino)benzoic acid (321 mg, 25%) as a pale yellow solid.

¹H NMR (400 MHz, DMSO-d₆) δ 12.93 (s, 1H), 8.67 (s, 1H), 8.61-8.60 (m, 1H), 8.05-8.00 (m, 2H), 7.90-7.86 (m, 1H), 7.85-7.80 (m, 1H), 7.73-7.71 (m, 1H), 7.45-7.37 (m, 3H), 7.28-7.24 (m, 1H), 7.21-7.16 (m, 2H).

### Synthesis of 3-((4-(pyridin-3-yl)phenyl)amino)benzoic acid

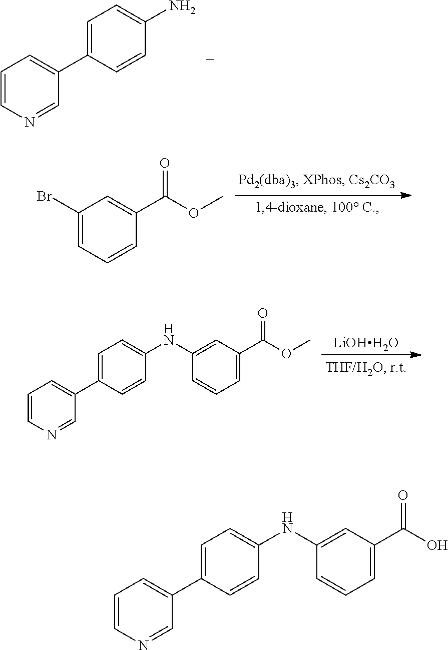

Step 1: To a solution of methyl 3-bromobenzoate (2.18 g, 10.14 mmol) in 1,4-dioxane (46 mL) was 4-(pyridin-3-yl)aniline (1.57 g, 9.22 mmol), XPhos (0.75 g, 1.56 mmol), and cesium carbonate (6.0 g, 18.44 mmol). Pd₂(dba)₃ (0.68 g, 0.74 mmol) was added into the solution. The solution was stirred for 16 hours at 100° C. The reaction mixture was concentrated and purified by MPLC. The crude mixture was solidified by using acetone to give methyl 3-((4-(pyridin-3-yl)phenyl)amino)benzoate (1.0 g, 36%) as a pale yellow solid.

¹H NMR (400 MHz, DMSO-d₆) δ 8.89-8.85 (m, 1H), 8.67 (s, 1H), 8.53-8.48 (m, 1H), 8.05-8.00 (m, 1H), 7.73-7.65 (m, 3H), 7.48-7.37 (m, 4H), 7.24-7.19 (m, 2H), 3.85 (s, 3H).

Step 2: Methyl 3-((4-(pyridin-3-yl)phenyl)amino)benzoate (0.35 g, 1.15 mmol) and LiOH.H₂O (0.19 g, 4.6 mmol) were mixed in THF/H₂O (8/4 mL) and stirred for 24 hours at room temperature. The reaction mixture was extracted by EA and aq. HCl (1N). The organic layer was dried over anhydrous MgSO₄ and concentrated. The residue was purified by MPLC to give crude 3-((4-(pyridin-3-yl)phenyl)amino)benzoic acid (125 mg, 37%) as a yellow solid.

¹H NMR (400 MHz, DMSO-d₆) δ 8.88 (s, 1H), 8.62 (s, 1H), 8.50 (d, J=4.0 Hz, 1H), 8.06-8.02 (m, 1H), 7.70-7.64 (m, 2H), 7.47-7.30 (m, 5H), 7.24-7.18 (m, 2H).

### Synthesis of 3-((4-(pyridin-4-yl)phenyl)amino)benzoic acid

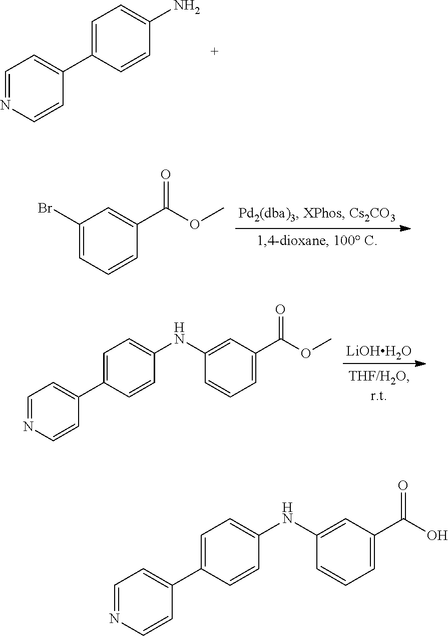

Step 1: To a solution of methyl 3-bromobenzoate (2.18 g, 10.14 mmol) in 1,4-dioxane (46 mL) was 4-(pyridin-4-yl)aniline (1.57 g, 9.22 mmol), XPhos (0.75 g, 1.56 mmol), and cesium carbonate (6.0 g, 18.44 mmol). Pd₂(dba)₃ (0.68 g, 0.74 mmol) was added into the solution. The solution was stirred for 16 hours at 100° C. The reaction mixture was concentrated and purified by MPLC. The crude mixture was solidified by using acetone to give methyl 3-((4-(pyridin-4-yl)phenyl)amino)benzoate (1.0 g, 36%) as a pale yellow solid.

¹H NMR (400 MHz, DMSO-d₆) δ 8.79-8.77 (m, 1H), 8.59-8.56 (m, 2H), 7.80-7.76 (m, 2H), 7.74-7.71 (m, 1H), 7.69-7.66 (m, 2H), 7.48-7.41 (m, 3H), 7.23-7.19 (m, 2H), 3.86-3.85 (m, 3H).

Step 2: Methyl 3-((4-(pyridin-4-yl)phenyl)amino)benzoate (0.76 g, 2.5 mmol) and LiOH.H₂O (0.42 g, 10 mmol) were mixed in THF/H₂O (17/8.5 mL) and stirred for 40 hours at room temperature. The reaction mixture was extracted by EA and aq. HCl (1N). The organic layer was dried over anhydrous MgSO₄ and concentrated. The crude mixture was solidified by using EA and acetone to give 3-((4-(pyridin-4-yl)phenyl)amino)benzoic acid (244 mg, 34%) as a yellow solid.

¹H NMR (400 MHz, DMSO-d₆) δ 12.99 (bs, 1H), 9.24 (s, 1H), 8.75 (s, 2H), 8.23-8.16 (m, 2H), 7.99 (d, J=8.8 Hz, 2H), 7.79 (s, 1H), 7.61-7.40 (m, 3H), 7.26 (d, J=42.4 Hz, 2H).

### Synthesis of 3-((4-(pyrimidin-2-yl)phenyl)amino)benzoic acid

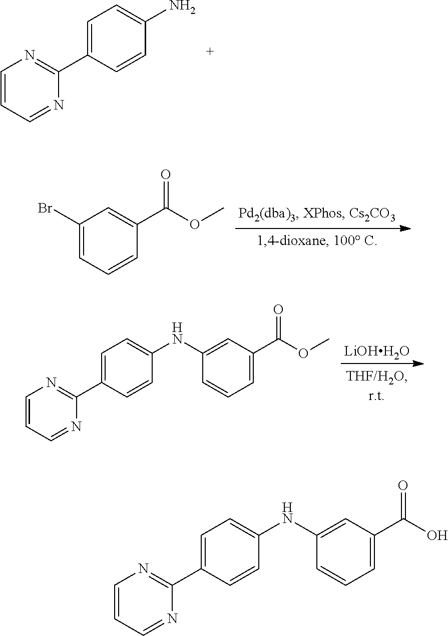

Step 1: To a solution of methyl 3-bromobenzoate (1.89 g, 8.8 mmol) in 1,4-dioxane (40 mL) was 4-(pyrimidin-2-yl)aniline (1.37 g, 8.0 mmol), XPhos (0.65 g, 1.36 mmol), and cesium carbonate (5.21 g, 16 mmol). Pd₂(dba)₃ (0.59 g, 0.64 mmol) was added into the solution. The solution was stirred for 16 hours at 100° C. The reaction mixture was concentrated and purified by MPLC. The crude mixture was solidified by using acetone to give methyl 3-((4-(pyrimidin-2-yl)phenyl)amino)benzoate (1.52 g, 62%) as a beige solid.

¹H NMR (400 MHz, DMSO-d₆) δ 8.85 (s, 1H), 8.83 (d, J=4.8 Hz, 2H), 8.33-8.29 (m, 2H), 7.76-7.74 (m, 1H), 7.52-7.42 (m, 3H), 7.33 (t, J=4.8 Hz, 1H), 7.22-7.17 (m, 2H), 3.86 (s, 3H).

Step 2: Methyl 3-((4-(pyrimidin-2-yl)phenyl)amino)benzoate (1.50 g, 4.91 mmol) and LiOH.H₂O (0.83 g, 19.65 mmol) were mixed in THF/H₂O (32/16 mL) and stirred for 40 hours at room temperature. The reaction mixture was extracted by EA and aq. HCl (1N). The organic layer was dried over anhydrous MgSO₄ and concentrated. The crude mixture was solidified by using EA and acetone to give 3-((4-(pyrimidin-2-yl)phenyl)amino)benzoic acid (1.10 g, 77%) as a beige solid.

¹H NMR (400 MHz, DMSO-d₆) δ 12.91 (bs, 1H), 8.88-8.79 (m, 3H), 8.32-8.29 (m, 2H), 7.74 (s, 1H), 7.51-7.46 (m, 1H), 7.46-7.39 (m, 2H), 7.32 (t, J=4.8 Hz, 1H), 7.21-7.17 (m, 2H).

### Synthesis of 3-((4-(pyrazin-2-yl)phenyl)amino)benzoic acid

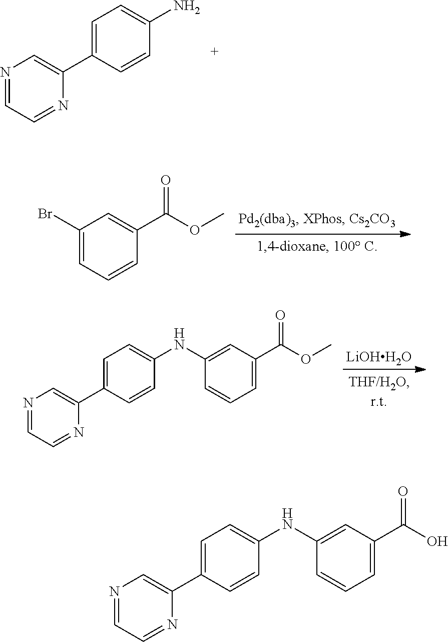

Step 1: To a solution of methyl 3-bromobenzoate (1.80 g, 8.35 mmol) in 1,4-dioxane (38 mL) was 4-(pyrazin-2-yl)aniline (1.30 g, 7.59 mmol), XPhos (0.62 g, 1.29 mmol), and cesium carbonate (4.95 g, 15.2 mmol). Pd₂(dba)₃ (0.56 g, 0.61 mmol) was added into the solution. The solution was stirred for 16 hours at 100° C. The reaction mixture was concentrated and purified by MPLC. The crude mixture was solidified by using acetone to give methyl 3-((4-(pyrazin-2-yl)phenyl)amino)benzoate (1.60 g, 69%) as a brown solid.

¹H NMR (400 MHz, DMSO-d₆) δ 9.19 (d, J=1.4 Hz, 1H), 8.83 (s, 1H), 8.65-8.63 (m, 1H), 8.50 (d, J=2.5 Hz, 1H), 8.11-8.07 (m, 2H), 7.76-7.74 (m, 1H), 7.53-7.40 (m, 3H), 7.22 (d, J=8.8 Hz, 2H), 3.85 (s, 3H).

Step 2: Methyl 3-((4-(pyrazin-2-yl)phenyl)amino)benzoate (1.58 g, 5.74 mmol) and LiOH.H₂O (0.87 g, 20.7 mmol) were mixed in THF/H₂O (38/19 mL) and stirred for 64 hours at room temperature. The reaction mixture was extracted by EA and aq. HCl (1N). The organic layer was dried over anhydrous MgSO₄ and concentrated. The residue was purified by MPLC to give 3-((4-(pyrazin-2-yl)phenyl)amino)benzoic acid (1.72 g, >99%) as a yellow solid.

¹H NMR (400 MHz, DMSO-d₆) δ 12.83 (s, 1H), 9.18 (d, J=1.5 Hz, 1H), 8.78 (s, 1H), 8.65-8.63 (m, 1H), 8.50 (d, J=2.5 Hz, 1H), 8.08 (d, J=8.8 Hz, 2H), 7.75 (s, 1H), 7.50-7.38 (m, 3H), 7.23-7.19 (m, 2H).

### Synthesis of 3-((4-(pyrimidin-5-yl)phenyl)amino)benzoic acid

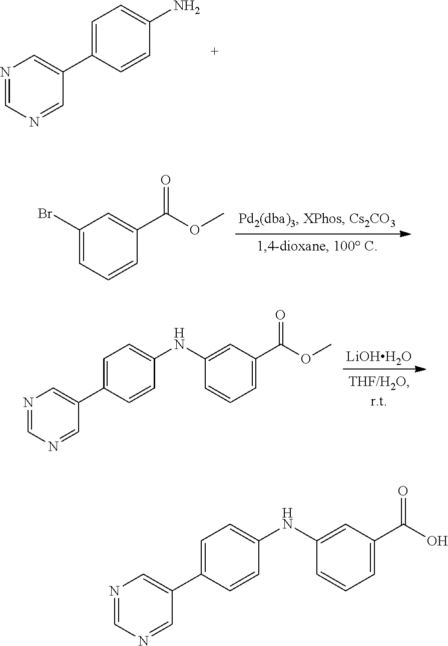

Step 1: To a solution of methyl 3-bromobenzoate (2.0 g, 9.32 mmol) in 1,4-dioxane (43 mL) was 4-(pyrimidin-5-yl)aniline (1.45 g, 8.47 mmol), XPhos (0.69 g, 1.44 mmol), and cesium carbonate (5.52 g, 16.94 mmol). Pd₂(dba)₃ (0.62 g, 0.68 mmol) was added into the solution. The solution was stirred for 16 hours at 100° C. The reaction mixture was concentrated and purified by MPLC. The crude mixture was solidified by using acetone to give methyl 3-((4-(pyrimidin-5-yl)phenyl)amino)benzoate (0.93 g, 36%) as a brown solid.

¹H NMR (400 MHz, DMSO-d₆) δ 9.12 (s, 3H), 8.75 (s, 1H), 7.79-7.72 (m, 3H), 7.50-7.36 (m, 3H), 7.25-7.22 (m, 2H), 3.85 (s, 3H).

Step 2: Methyl 3-((4-(pyrimidin-5-yl)phenyl)amino)benzoate (0.90 g, 2.95 mmol) and LiOH.H₂O (0.5 g, 20.7 mmol) were mixed in THF/H₂O (20/10 mL) and stirred for 64 hours at room temperature. The reaction mixture was extracted by EA and aq. HCl (1N). The organic layer was dried over anhydrous MgSO₄ and concentrated. The residue was purified by MPLC to give 3-((4-(pyrimidin-5-yl)phenyl)amino)benzoic acid (706 mg, 82%) as a yellow solid.

¹H NMR (400 MHz, DMSO-d₆) δ 12.91 (bs, 1H), 9.12-9.11 (m, 3H), 8.70 (s, 1H), 7.79-7.70 (m, 3H), 7.49-7.33 (m, 3H), 7.26-7.20 (m, 2H).

### Synthesis of 3-((4-(pyrimidin-4-yl)phenyl)amino)benzoic acid

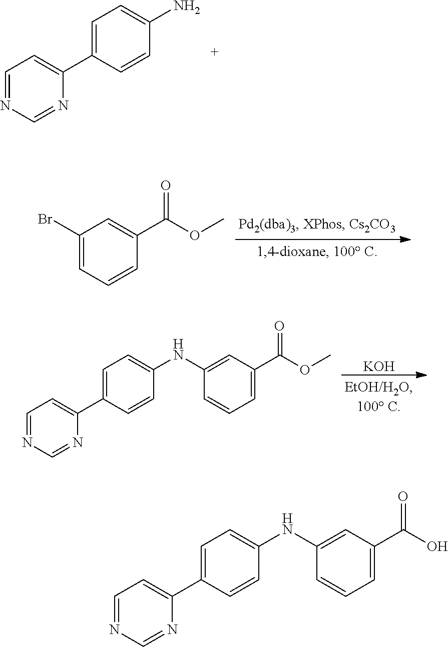

Step 1: To a solution of methyl 3-bromobenzoate (2.14 g, 9.93 mmol) in 1,4-dioxane (15 mL) was 4-(pyrimidin-4-yl)aniline (1.70 g, 9.93 mmol), BrettPhos (1.07 g, 1.99 mmol), and cesium carbonate (8.09 g, 24.8 mmol). Pd₂(dba)₃ (0.91 g, 0.99 mmol) was added into the solution. The solution was stirred for 12 hours at 100° C. under N₂ atmosphere. TLC indicated 4-(pyrimidin-4-yl)aniline was consumed completely and one new spot formed. The reaction was clean according to TLC. The reaction mixture was diluted with H₂O and extracted with EA. The combined organic layers were washed with brine, dried over Na₂SO₄, filtered, and concentrated under reduced pressure to give a residue. The residue was purified by column chromatography to methyl 3-((4-(pyrimidin-4-yl)phenyl)amino)benzoate (1.30 g, 43%) as a yellow solid.

¹H NMR (400 MHz, DMSO-d₆) δ 9.13 (s, 1H), 8.91 (s, 1H), 8.74 (d, J=5.20 Hz, 1H), 8.15 (d, J=8.80 Hz, 2H), 7.96 (d, J=5.20 Hz, 1H), 7.76 (s, 1H), 7.52-7.49 (m, 1H), 7.46-7.43 (m, 2H), 7.20 (d, J=8.80 Hz, 2H), 3.85 (s, 3H).

Step 2: Methyl 3-((4-(pyrimidin-4-yl)phenyl)amino)benzoate (1.30 g, 4.26 mmol) and KOH (478 mg, 8.52 mmol) were mixed in EtOH/H₂O (7/5 mL) and stirred for 4 hours at 100° C. TLC indicated methyl 3-((4-(pyrimidin-4-yl)phenyl)amino)benzoate was consumed completely and one new spot formed. The reaction was clean according to TLC. The reaction mixture was diluted with H₂O and extracted with 2-methyltetrahydrofuran and the pH was adjusted to 5-6 with 0.5 M HCl for aqueous phase. The resulting solution was extracted with 2-methyltetrahydrofuran. The combined organic layers were washed with brine, dried over Na₂SO₄, filtered, and concentrated under reduced pressure to give a residue. The crude product was triturated with acetonitrile for 12 hours at room temperature. 3-((4-(Pyrimidin-4-yl)phenyl)amino)benzoic acid (1.01 g, 97%) as a yellow solid.

¹H NMR (400 MHz, DMSO-d₆) δ 12.96 (bs, 1H), 9.13 (s, 1H), 8.88 (s, 1H), 8.74 (d, J=5.60 Hz, 1H), 8.15 (d, J=8.80 Hz, 2H), 7.97-7.94 (m, 1H), 7.75 (s, 1H), 7.52-7.48 (m, 1H), 7.43-7.41 (m, 2H), 7.20 (d, J=8.80 Hz, 2H).

### Synthesis of 2-((5-phenylpyridin-2-yl)amino)isonicotinic acid

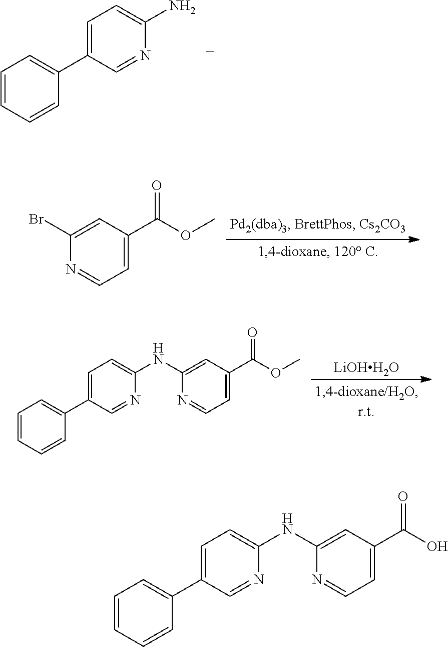

Step 1: 5-Phenylpyridin-2-amine (350 mg, 2.1 mmol), methyl 2-bromoisonicotinate (620 mg, 2.47 mmol), Pd₂(dba)₃ (188 mg, 0.21 mmol), BrettPhos (221 mg, 0.41 mmol), and cesium carbonate (1.3 g, 4.1 mmol) were mixed in 1,4-dioxane (10 mL) and heated in a microwave reactor for 90 minutes at 120° C. The reaction mixture was extracted by EA and brine. The crude mixture was solidified by using EA and HEX to give methyl 2-((5-phenylpyridin-2-yl)amino)isonicotinate (393 mg, 63%) as an orange solid.

¹H NMR (400 MHz, DMSO-d₆) δ 10.16 (s, 1H), 8.62 (d, J=2.5 Hz, 1H), 8.43 (d, J=5.1 Hz, 1H), 8.33 (s, 1H), 8.04 (dd, J=2.5, 8.8 Hz, 1H), 7.85-7.82 (m, 1H), 7.71 (d, J=7.3 Hz, 2H), 7.48 (t, J=7.7 Hz, 2H), 7.39-7.30 (m, 2H), 3.91 (s, 3H).

Step 2: Methyl 2-((5-phenylpyridin-2-yl)amino)isonicotinate (350 mg, 1.15 mmol) and LiOH.H₂O (481 mg, 11.5 mmol) were mixed in H₂O/1,4-dioxane (4.8/23 mL) and stirred for 18 hours at room temperature. Then pH value of the solution was adjusted to 1-2 by 1 N HCl. The yellow solid was precipitated out of the solution, and the solution was filtered to give 2-((5-phenylpyridin-2-yl)amino)isonicotinic acid (190 mg, 57%) as a yellow solid.

¹H NMR (400 MHz, DMSO-d₆) δ 13.90 (s, 1H), 11.70 (s, 1H), 8.65 (d, J=2.4 Hz, 1H), 8.51 (d, J=5.4 Hz, 1H), 8.34 (d, J=8.0 Hz, 1H), 8.06 (s, 1H), 7.75-7.71 (m, 3H), 7.56-7.49 (m, 3H), 7.43 (t, J=7.4 Hz, 1H).

### Synthesis of 2-((5-phenylpyrimidin-2-yl)amino)isonicotinic acid

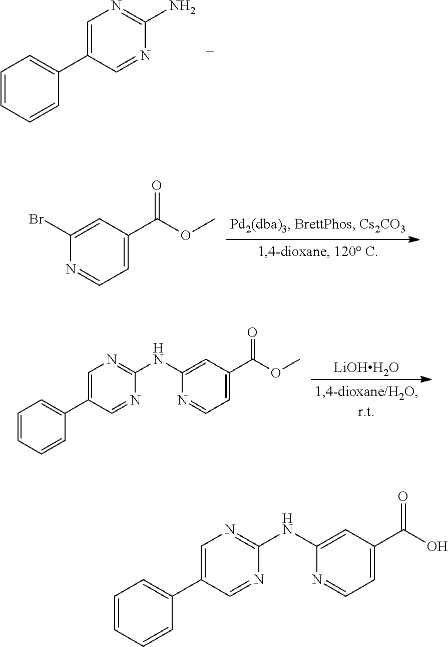

Step 1: 5-Phenylpyrimidin-2-amine (500 mg, 2.9 mmol), methyl 2-bromoisonicotinate (620 mg, 2.47 mmol), Pd₂(dba)₃ (267 mg, 0.29 mmol), BrettPhos (313 mg, 0.58 mmol), and cesium carbonate (1.9 g, 5.8 mmol) were mixed in 1,4-dioxane (15 mL) and heated in a microwave reactor for 90 minutes at 120° C. The reaction mixture was extracted by DCM and brine. The crude mixture was solidified by using EA to give methyl 2-((5-phenylpyrimidin-2-yl)amino)isonicotinate (609 mg, 68%) as an yellow solid.

¹H NMR (400 MHz, DMSO-d₆) δ 10.42 (s, 1H), 8.98 (s, 2H), 8.84 (s, 1H), 8.50 (d, J=5.0 Hz, 1H), 7.79 (d, J=7.4 Hz, 2H), 7.51 (dd, J=7.7, 7.7 Hz, 2H), 7.46-7.39 (m, 2H), 3.93 (s, 3H).

Step 2: Methyl 2-((5-phenylpyrimidin-2-yl)amino)isonicotinate (550 mg, 1.8 mmol) and LiOH.H₂O (753 mg, 18 mmol) were mixed in H₂O/1,4-dioxane (7.5/36 mL) and stirred for 18 hours at room temperature. Then pH value of the solution was adjusted to 3 by 1 N HCl. The reaction mixture was extracted by EA and brine. The crude mixture was solidified by using EA and HEX to give 2-((5-phenylpyrimidin-2-yl)amino)isonicotinic acid (153 mg, 29%) as a beige solid.

¹H NMR (400 MHz, DMSO-d₆) δ 13.60 (s, 1H), 10.34 (s, 1H), 8.97 (s, 2H), 8.84 (s, 1H), 8.47 (d, J=5.0 Hz, 1H), 7.80-7.77 (m, 2H), 7.51 (dd, J=7.6, 7.6 Hz, 2H), 7.44-7.38 (m, 2H).

### Synthesis of 5-((5-phenylpyrimidin-2-yl)amino)nicotinic acid

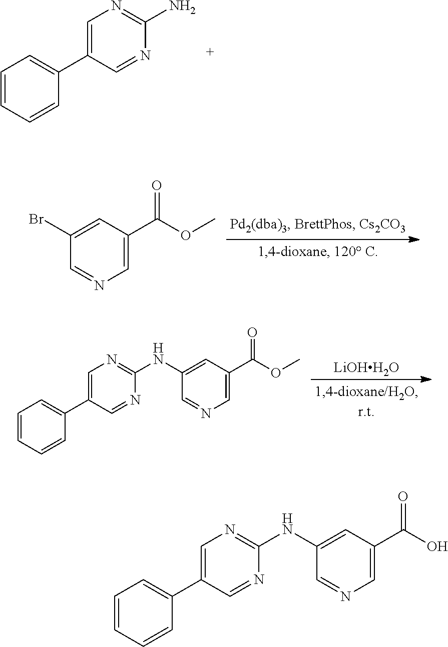

Step 1: 5-Phenylpyrimidin-2-amine (500 mg, 2.9 mmol), methyl 5-bromonicotinate (757 mg, 3.5 mmol), Pd₂(dba)₃ (267 mg, 0.29 mmol), BrettPhos (313 mg, 0.58 mmol), and cesium carbonate (1.9 g, 5.8 mmol) were mixed in 1,4-dioxane (15 mL) and heated in a microwave reactor for 90 minutes at 120° C. The reaction mixture was extracted by EA and brine. The grey solid was precipitated out of the solution, and the solution was filtered to give methyl 5-((5-phenylpyrimidin-2-yl)amino)nicotinate (630 mg, 70%) as a grey solid.

¹H NMR (400 MHz, DMSO-d₆) δ 10.28 (s, 1H), 9.16 (d, J=2.6 Hz, 1H), 8.95 (s, 2H), 8.87 (dd, J=2.3, 2.3 Hz, 1H), 8.70 (d, J=1.9 Hz, 1H), 7.76 (d, J=7.3 Hz, 2H), 7.50 (dd, J=7.6, 7.6 Hz, 2H), 7.40 (t, J=7.4 Hz, 1H), 3.91 (s, 3H).

Step 2: Methyl 5-((5-phenylpyrimidin-2-yl)amino)nicotinate (620 mg, 2 mmol) and LiOH.H₂O (849 mg, 20 mmol) were mixed in H₂O/1,4-dioxane (8.4/40 mL) and stirred for 18 hours at room temperature. Then pH value of the solution was adjusted to 2 by 1 N HCl. The grey solid was precipitated out of the solution, and the solution was filtered to give 5-((5-phenylpyrimidin-2-yl)amino)nicotinic acid (497 mg, 84%) as a grey solid.

¹H NMR (400 MHz, DMSO-d₆) δ 10.22 (s, 1H), 9.11 (d, J=2.6 Hz, 1H), 8.94 (s, 2H), 8.83 (dd, J=2.1, 2.1 Hz, 1H), 8.67 (d, J=1.8 Hz, 1H), 7.76 (d, J=7.4 Hz, 2H), 7.50 (dd, J=7.6, 7.6 Hz, 2H), 7.40 (t, J=7.4 Hz, 1H).

### Synthesis of 4-((5-phenylpyrimidin-2-yl)amino)picolinic acid

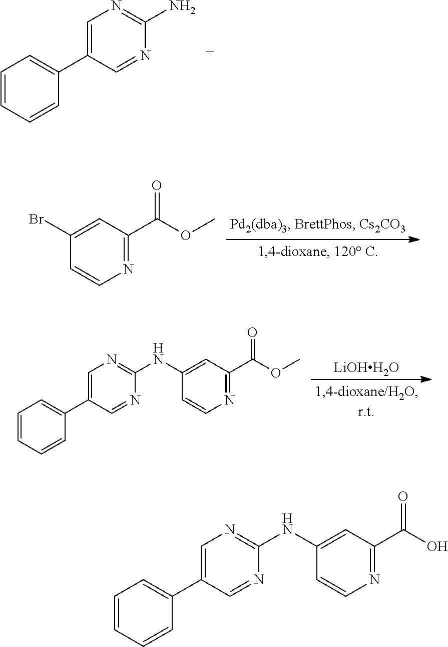

Step 1: 5-Phenylpyrimidin-2-amine (500 mg, 2.9 mmol), methyl 4-bromopicolinate (757 mg, 3.5 mmol), Pd₂(dba)₃ (267 mg, 0.29 mmol), BrettPhos (313 mg, 0.58 mmol), and cesium carbonate (1.9 g, 5.8 mmol) were mixed in 1,4-dioxane (15 mL) and heated in a microwave reactor for 90 minutes at 120° C. The reaction mixture was extracted by EA and brine. The beige solid was precipitated out of the solution, and the solution was filtered to give methyl 4-((5-phenylpyrimidin-2-yl)amino)picolinate (417 mg, 47%) as a beige solid.

¹H NMR (400 MHz, DMSO-d₆) δ 10.54 (s, 1H), 9.00 (s, 2H), 8.54 (d, J=2.3 Hz, 1H), 8.51 (d, J=5.6 Hz, 1H), 8.06 (dd, J=2.3, 5.6 Hz, 1H), 7.78 (d, J=7.4 Hz, 2H), 7.51 (dd, J=7.6, 7.6 Hz, 2H), 7.42 (t, J=7.3 Hz, 1H), 3.89 (s, 3H).

Step 2: Methyl 4-((5-phenylpyrimidin-2-yl)amino)picolinate (400 mg, 1.3 mmol) and LiOH.H₂O (548 mg, 13 mmol) were mixed in H₂O/1,4-dioxane (5.4/26 mL) and stirred for 18 hours at room temperature. Then pH value of the solution was adjusted to 1 by 1 N HCl. The beige solid was precipitated out of the solution, and the solution was filtered to give 4-((5-phenylpyrimidin-2-yl)amino)picolinic acid (346 mg, 92%) as a beige solid.

¹H NMR (400 MHz, DMSO-d₆) δ 10.78 (s, 1H), 9.04-9.03 (m, 2H), 8.56 (d, J=2.3 Hz, 1H), 8.47 (d, J=5.8 Hz, 1H), 8.09 (dd, J=2.3, 6.0 Hz, 1H), 7.80 (d, J=7.3 Hz, 2H), 7.52 (t, J=7.6 Hz, 2H), 7.43 (t, J=7.3 Hz, 1H).

### Synthesis of 3-((5-phenyl-1,3,4-oxadiazol-2-yl)amino)benzoic acid

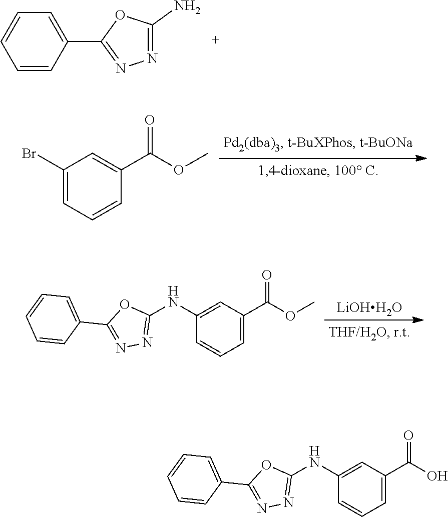

Step 1: To a solution of methyl 3-bromobenzoate (0.95 g, 4.4 mmol) in 1,4-dioxane (8 mL) was added 5-phenyl-1,3,4-oxadiazol-2-amine (0.65 g, 4.0 mmol), t-BuXPhos (0.29 g, 0.68 mmol), and t-BuONa (0.77 g, 8.0 mmol). Pd₂(dba)₃ (0.29 g, 0.32 mmol) was added into the solution. The solution was stirred for 16 hours at 100° C. The reaction mixture was concentrated and purified by MPLC. The crude mixture was solidified by using acetone to give methyl 3-((5-phenyl-1,3,4-oxadiazol-2-yl)amino)benzoate (0.33 g, 23%) as a beige solid.

¹H NMR (400 MHz, DMSO-d₆) δ 10.99 (bs, 1H), 8.33 (dd, J=1.8, 1.8 Hz, 1H), 7.94-7.90 (m, 2H), 7.89-7.85 (m, 1H), 7.68-7.58 (m, 4H), 7.54 (dd, J=7.9, 7.9 Hz, 1H), 3.89 (s, 3H).

Step 2: Methyl 3-((5-phenyl-1,3,4-oxadiazol-2-yl)amino)benzoate (0.32 g, 1.08 mmol) and LiOH.H₂O (0.18 g, 4.32 mmol) were mixed in THF/H₂O (7.2/3.6 mL) and stirred for 24 hours at room temperature. The reaction mixture was extracted by EA and aq. HCl (1N). The organic layer was dried over anhydrous MgSO₄ and concentrated to give crude 3-((5-phenyl-1,3,4-oxadiazol-2-yl)amino)benzoic acid (125 mg, 41%) as a pale brown solid.

¹H NMR (400 MHz, DMSO-d₆) δ 10.95 (s, 1H), 8.29 (s, 1H), 7.97-7.88 (m, 2H), 7.85 (dd, J=1.4, 8.0 Hz, 1H), 7.67-7.47 (m, 5H).

2) Substitution A

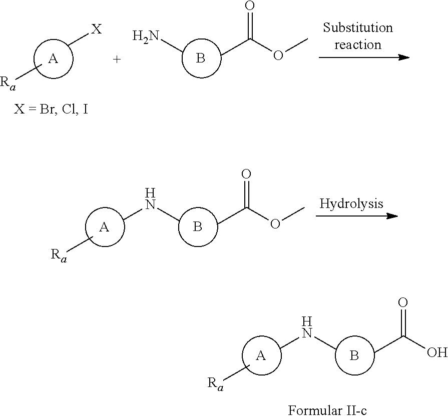

### Synthesis of (1s,4s)-4-((6-phenylpyridazin-3-yl)amino)bicyclo[2.2.1]heptane-1-carboxylic acid

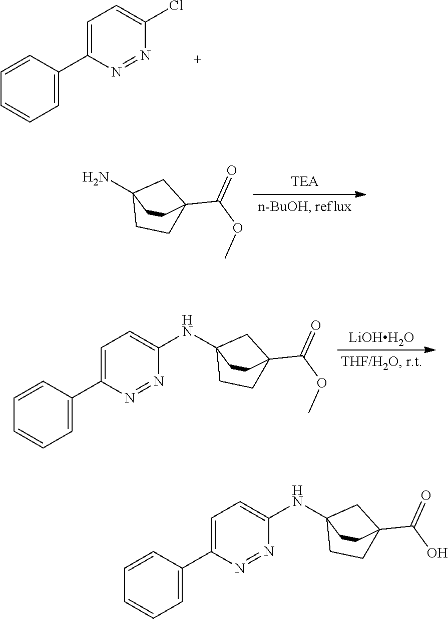

Step 1: In a sealed tube, 3-chloro-6-phenylpyridazine (500 mg, 2.6 mmol) and methyl (1s,4s)-4-aminobicyclo[2.2.1]heptane-1-carboxylate (578 mg, 3.4 mmol) were mixed in n-butanol (10 mL). To this reaction mixture, trifluoroacetic acid (75 mg, 0.65 mmol) was added at room temperature and allowed to stir for 72 hours at 150° C. Progress of the reaction was monitored by TLC. Reaction was cooled to r.t., water was added, and product was extracted with EA. The combined organic layer was washed with water and brine, dried over anhydrous Na₂SO₄, and concentrated under vacuum to provide crude product, which was purified by combi-flash column chromatography. Product was eluted out in 15% EA in HEX to provide methyl (1s,4s)-4-((6-phenylpyridazin-3-yl)amino)bicyclo[2.2.1]heptane-1-carboxylate (170 mg, 20%) as a white solid. m/z 324.

¹H NMR (400 MHz, methanol-d₄) δ 7.88 (d, J=7.0 Hz, 1H), 7.73 (d, J=9.5 Hz, 1H), 7.55-7.38 (m, 3H), 6.96 (d, J=9.5 Hz, 1H), 3.71 (s, 3H), 2.34-2.08 (m, 6H), 2.03-1.95 (m, 2H), 1.87-1.74 (m, 2H).

Step 2: Methyl (1s,4s)-4-((6-phenylpyridazin-3-yl)amino)bicyclo[2.2.1]heptane-1-carboxylate (1.4 g, 4.3 mmol) was dissolved in tetrahydrofuran: H₂O (2:1, 15 mL) and lithium hydroxide (541 mg, 12.9 mmol) was added at 0° C. and reaction was allowed to stir for 6 hours at room temperature. Progress of the reaction was monitored by TLC. After completion of reaction, 2N HCl solution was added until pH 4 adjusted and precipitates were filtered and dried to provide (1s,4s)-4-((6-phenylpyridazin-3-yl)amino)bicyclo[2.2.1]heptane-1-carboxylic acid (1.05 g, 78%) as an off white solid.

¹H NMR (400 MHz, DMSO-d₆) δ 12.16 (s, 1H), 7.99 (d, J=8.1 Hz, 2H), 7.87 (d, J=9.6 Hz, 1H), 7.56-7.38 (m, 4H), 7.01 (d, J=8.8 Hz, 1H), 2.21-1.97 (m, 6H), 1.90-1.79 (m, 2H), 1.76-1.63 (m, 2H).

3) Substitution B

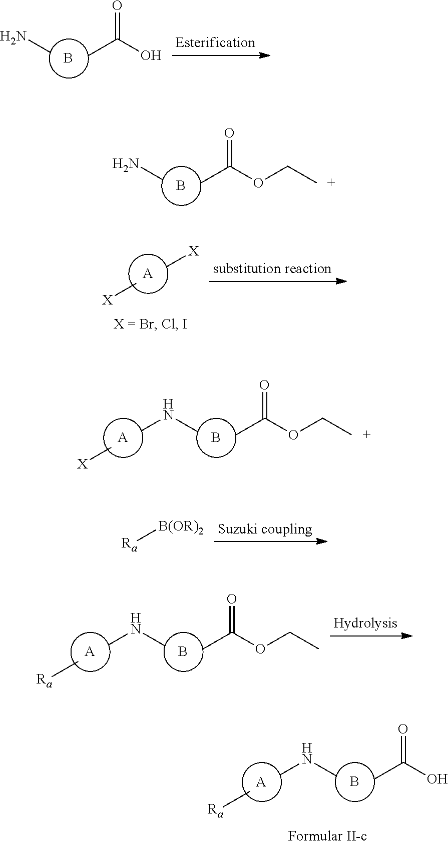

### Synthesis of 3-((6-phenylpyridazin-3-yl)amino)adamantane-1-carboxylic acid

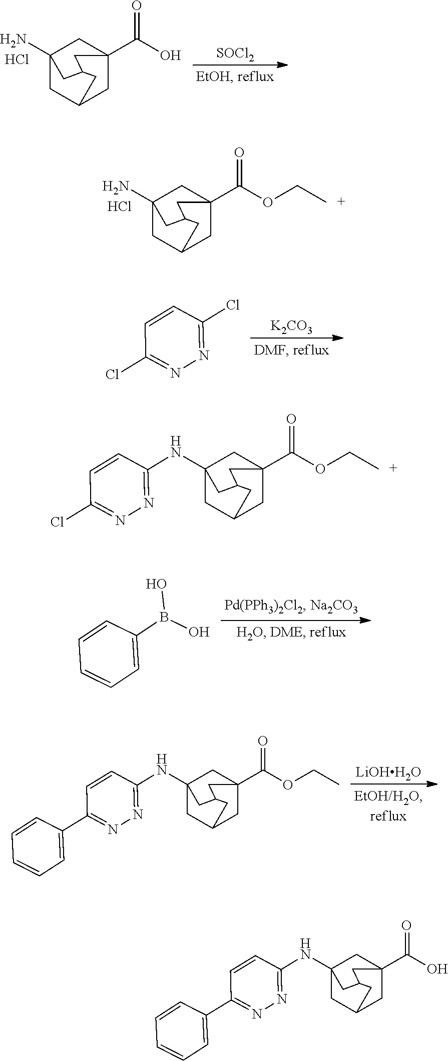

Step 1: To a solution of 3-aminoadamantane-1-carboxylic acid hydrochloride (20 g, 86 mmol) in EtOH (140 mL) was added SOCl₂ (10.3 g, 86.3 mmol) at room temperature. The reaction mixture was stirred for 4 hours at 80° C. Liquid chromatography-mass spectrometry (LCMS) showed the reaction was completed. The reaction mixture was concentrated under reduced pressure to give a residue. Petroleum ether was then added, and the mixture was once again concentrated under reduced pressure at which point a solid began to precipitate, the process was repeated three more times. The crude product was triturated with Petroleum ether for 30 minutes at room temperature and the suspension was filtered to give ethyl 3-aminoadamantane-1-carboxylate hydrochloride (21 g, 94%) as a white solid. m/z 224.

¹H NMR (400 MHz, DMSO-d₆) δ 8.28 (s, 3H), 4.06 (q, J=7.2 Hz, 2H), 2.18 (s, 2H), 1.90 (s, 2H), 1.77 (s, 6H), 1.67-1.55 (m, 4H), 1.17 (t, J=7.2 Hz, 3H).

Step 2: To a solution of 3,6-dichloropyridazine (24 g, 162 mmol) in DMF (147 mL) was added ethyl 3-aminoadamantane-1-carboxylate hydrochloride (21.0 g, 80.8 mmol) and K₂CO₃ (33.5 g, 243 mmol) at room temperature. The reaction mixture was stirred for 12 hours at 135° C. TLC showed the ˜50% of 3,6-dichloropyridazine remained and ˜20% of product was detected. The residue was diluted with water and extracted with EA. The combined organic layers were washed with brine, dried over Na₂SO₄, filtered, and concentrated under reduced pressure to give a residue. The residue was purified by column chromatography to give ethyl 3-((6-chloropyridazin-3-yl)amino)adamantane-1-carboxylate (1.80 g, 7%) as a white solid.

¹H NMR (400 MHz, CDCl₃) δ 7.11 (d, J=9.2 Hz, 1H), 6.59 (d, J=9.2 Hz, 1H), 4.44 (s, 1H), 4.14-3.93 (m, 2H), 2.25 (s, 4H), 2.18-2.08 (m, 4H), 1.92-1.88 (m, 4H), 1.70-1.68 (m, 2H), 1.26-1.22 (m, 3H).

Step 3: To a solution of ethyl 3-((6-chloropyridazin-3-yl)amino)adamantane-1-carboxylate (1.8 g, 5.4 mmol) in dimethyl ether (DME) (9 mL) and H₂O (1.8 mL) was added phenylboronic acid (719 mg, 5.9 mmol) and Na₂CO₃ (2.84 g, 26.8 mmol) at room temperature. Pd(PPh₃)₂Cl₂ (376 mg, 0.54 mmol) was added into above mixture at room temperature. The suspension was degassed under vacuum and purged with N₂ three times, and the reaction mixture was stirred for 12 hours at 80° C. TLC showed the reaction was completed. The residue was diluted with H₂O and extracted with EA. The combined organic layers were washed with brine, dried over Na₂SO₄, filtered, and concentrated under reduced pressure to give a residue. The residue was purified by column chromatography to give product. The residue was purified by preparative HPLC (prep-HPLC) to give desired compound. Ethyl 3-((6-phenylpyridazin-3-yl)amino)adamantane-1-carboxylate (800 mg, 40%) was obtained as a white solid.

¹H NMR (400 MHz, CDCl₃) δ 7.97 (d, J=7.2 Hz, 2H), 7.56 (d, J=9.6 Hz, 1H), 7.48-7.44 (m 2H), 7.42-7.40 (m 1H), 6.70 (d, J=9.2 Hz, 1H), 4.50 (bs, 1H), 4.15-4.09 (m, 2H), 2.33 (s, 2H), 2.28 (s, 2H), 2.19 (s, 3H), 1.90 (q, J=12 Hz, 3H), 1.75-1.70 (m, 2H), 1.59 (s, 2H), 1.29-1.23 (m, 3H).

Step 4: To a solution of ethyl 3-((6-phenylpyridazin-3-yl)amino)adamantane-1-carboxylate (800 mg, 2.12 mmol) in EtOH (3.2 mL) was added H₂O (1.6 mL) and LiOH.H₂O (445 mg, 10.6 mmol) at room temperature. The reaction mixture was stirred for 12 hours at 40˜ 45° C. LCMS showed the reaction was completed. The reaction mixture was concentrated under reduced pressure to remove EtOH. The mixture was adjusted to pH 5˜6 with citric acid solution and white solid was precipitated, the suspension was filtered and the filter cake was concentrated under reduced pressure to give 3-((6-phenylpyridazin-3-yl)amino)adamantane-1-carboxylic acid (450 mg, 60%) as a white solid. m/z 350.

¹H NMR (400 MHz, DMSO-d₆) δ 12.11 (s, 1H), 7.96 (d, J=7.2 Hz, 2H), 7.75 (d, J=9.2 Hz, 1H), 7.48-7.44 (m, 2H), 7.40-7.38 (m, 1H), 6.90 (d, J=9.6 Hz, 1H), 6.60 (s, 1H), 2.26 (s, 2H), 2.18-2.16 (m, 4H), 2.05-2.03 (m, 2H), 1.82-1.79 (m, 4H), 1.66-1.63 (m, 2H).

### Example 2: Synthesis of Compounds

1. Synthesis by Method A

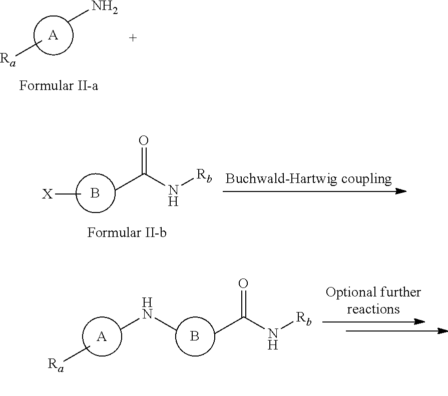

Synthesis of Compound 1

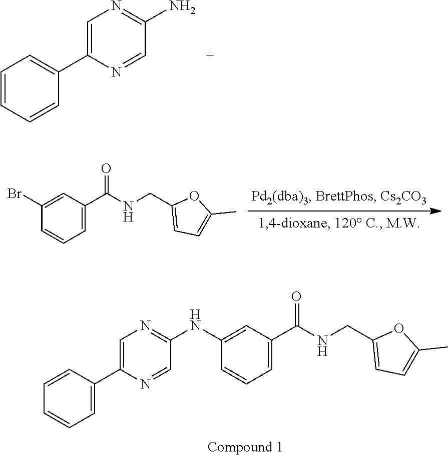 [SMILES: Nc1cnc(-c2ccccc2)cn1 ; "+" ; Cc1ccc(CNC(=O)c2cccc(Br)c2)o1 ; Cc1ccc(CNC(=O)c2cccc(Nc3cnc(-c4ccccc4)cn3)c2)o1]

5-Phenylpyrazin-2-amine (10 mg, 0.058 mmol), 3-bromo-N-((5-methylfuran-2-yl)methyl)benzamide (14.5 mg, 0.049 mmol), Pd₂(dba)₃ (0.9 mg, 0.00098 mmol), BrettPhos (5.3 mg, 0.0098 mmol), and cesium carbonate (32 mg, 0.098 mmol) were mixed in 1,4-dioxane (0.2 mL) and heated in a microwave reactor for 90 minutes at 120° C. The reaction mixture was extracted by EA and brine. The organic layer was dried over anhydrous Na₂SO₄ and concentrated. The residue was purified by MPLC to give compound 1, N-[(5-methylfuran-2-yl)methyl]-3-[(5-phenylpyrazin-2-yl)amino]benzamide (13 mg, 69%) as a yellow solid.

Synthesis of Compound 2

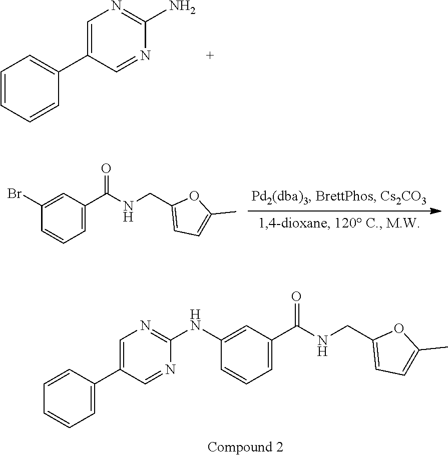 [SMILES: Nc1ncc(-c2ccccc2)cn1 ; "+" ; Cc1ccc(CNC(=O)c2cccc(Br)c2)o1 ; Cc1ccc(CNC(=O)c2cccc(Nc3ncc(-c4ccccc4)cn3)c2)o1]

5-Phenylpyrimidin-2-amine (10 mg, 0.058 mmol), 3-bromo-N-((5-methylfuran-2-yl)methyl)benzamide (14.5 mg, 0.049 mmol), Pd₂(dba)₃ (0.9 mg, 0.00098 mmol), BrettPhos (5.3 mg, 0.0098 mmol), and cesium carbonate (32 mg, 0.098 mmol) were mixed in 1,4-dioxane (0.2 mL) and heated in a microwave reactor for 90 minutes at 120° C. The reaction mixture was extracted by EA and brine. The organic layer was dried over anhydrous Na₂SO₄ and concentrated. The residue was purified by MPLC to give compound 2, N-[(5-methylfuran-2-yl)methyl]-3-[(5-phenylpyrimidin-2-yl)amino]benzamide (14 mg, 74%) as a white solid.

Synthesis of Compound 3

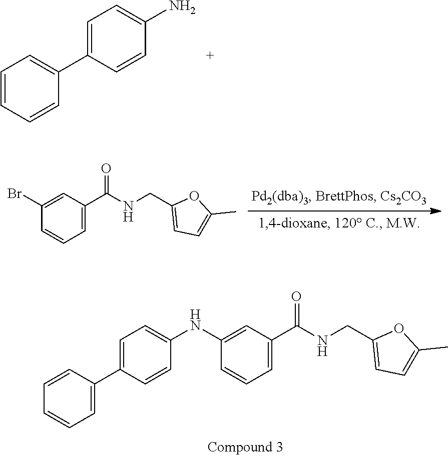 [SMILES: Nc1ccc(-c2ccccc2)cc1 ; "+" ; Cc1ccc(CNC(=O)c2cccc(Br)c2)o1 ; Cc1ccc(CNC(=O)c2cccc(Nc3ccc(-c4ccccc4)cc3)c2)o1]

[1,1′-Biphenyl]-4-amine (20 mg, 0.12 mmol), 3-bromo-N-((5-methylfuran-2-yl)methyl)benzamide (29 mg, 0.098 mmol), Pd₂(dba)₃ (1.8 mg, 0.002 mmol), BrettPhos (10.6 mg, 0.020 mmol), and cesium carbonate (64 mg, 0.2 mmol) were mixed in 1,4-dioxane (1 mL) and heated in a microwave reactor for 90 minutes at 120° C. The reaction mixture was extracted by EA and brine. The organic layer was dried over anhydrous Na₂SO₄ and concentrated. The residue was purified by MPLC to give compound 3, 3-({[1,1′-biphenyl]-4-yl}amino)-N-[(5-methylfuran-2-yl)methyl]benzamide (36.5 mg, 97%) as a yellowish white solid.

Synthesis of Compound 4

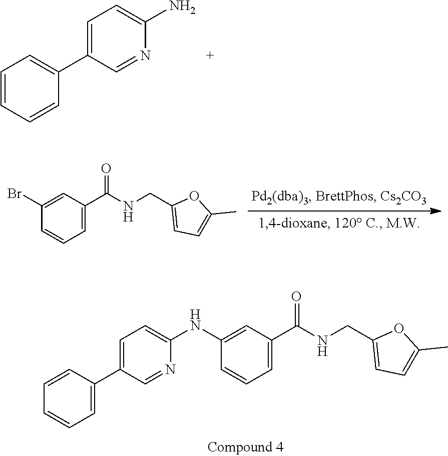 [SMILES: Nc1ccc(-c2ccccc2)cn1 ; "+" ; Cc1ccc(CNC(=O)c2cccc(Br)c2)o1 ; Cc1ccc(CNC(=O)c2cccc(Nc3ccc(-c4ccccc4)cn3)c2)o1]

5-Phenylpyridin-2-amine (20 mg, 0.12 mmol), 3-bromo-N-((5-methylfuran-2-yl)methyl)benzamide (29 mg, 0.098 mmol), Pd₂(dba)₃ (1.8 mg, 0.002 mmol), BrettPhos (10.6 mg, 0.020 mmol), and cesium carbonate (64 mg, 0.2 mmol) were mixed in 1,4-dioxane (1 mL) and heated in a microwave reactor for 90 minutes at 120° C. The reaction mixture was extracted by EA and brine. The organic layer was dried over anhydrous Na₂SO₄ and concentrated. The residue was purified by MPLC to give compound 4, N-[(5-methylfuran-2-yl)methyl]-3-[(5-phenylpyridin-2-yl)amino]benzamide (24 mg, 64%) as a yellowish white solid.

Synthesis of Compound 5

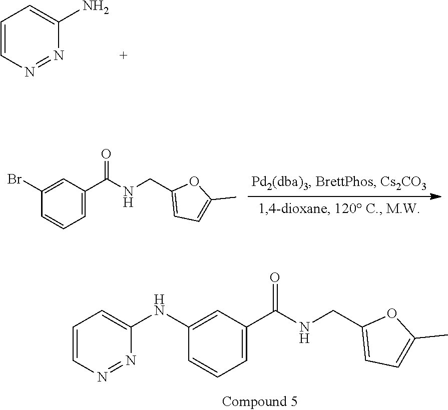 [SMILES: Nc1cccnn1 ; "+" ; Cc1ccc(CNC(=O)c2cccc(Br)c2)o1 ; Cc1ccc(CNC(=O)c2cccc(Nc3cccnn3)c2)o1]

Pyridazin-3-amine (15 mg, 0.16 mmol), 3-bromo-N-((5-methylfuran-2-yl)methyl)benzamide (39 mg, 0.13 mmol), Pd₂(dba)₃ (2.4 mg, 0.0026 mmol), BrettPhos (14 mg, 0.026 mmol), and cesium carbonate (86 mg, 0.26 mmol) were mixed in 1,4-dioxane (0.7 mL) and heated in a microwave reactor for 60 minutes at 120° C. The reaction mixture was concentrated and purified by MPLC to give compound 5, N-[(5-methylfuran-2-yl)methyl]-3-[(pyridazin-3-yl)amino]benzamide (17 mg, 42%) as a beige solid.

Synthesis of Compound 6

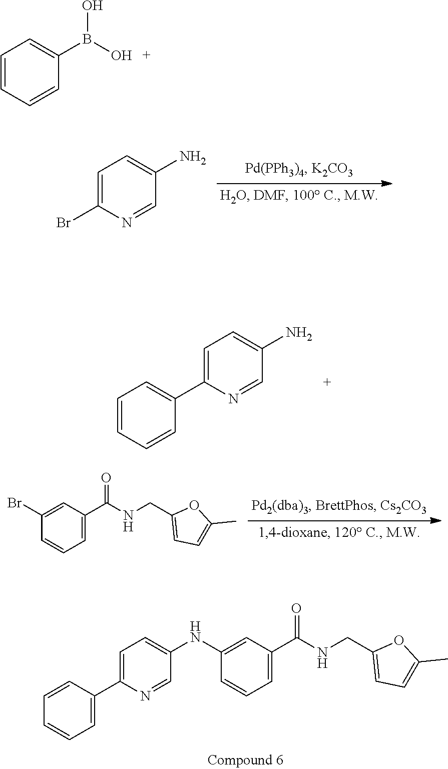

Step 1: Phenylboronic acid (500 mg, 4.1 mmol), 6-bromopyridin-3-amine (591 mg, 3.42 mmol), Pd(PPh₃)₄ (197 mg, 0.17 mmol), and potassium carbonate (1.7 g, 12.6 mmol) were mixed in H₂O/DMF (7/7 mL) and heated in a microwave reactor for 60 minutes at 100° C. The reaction mixture was extracted by EA and brine. The organic layer was dried over anhydrous Na₂SO₄ and concentrated. The reaction mixture was concentrated and purified by MPLC to give 6-phenylpyridin-3-amine (276 mg, 47%) as a yellow solid.

Step 2: 6-Phenylpyridin-3-amine (20 mg, 0.12 mmol), 3-bromo-N-((5-methylfuran-2-yl)methyl)benzamide (29 mg, 0.098 mmol), Pd₂(dba)₃ (1.8 mg, 0.002 mmol), BrettPhos (10.5 mg, 0.02 mmol), and cesium carbonate (64 mg, 0.2 mmol) were mixed in 1,4-dioxane (0.8 mL) and heated in a microwave reactor for 60 minutes at 120° C. The reaction mixture was concentrated and purified by MPLC to give compound 6, N-[(5-methylfuran-2-yl)methyl]-3-[(6-phenylpyridin-3-yl)amino]benzamide (34 mg, 91%) as a yellowish white solid.

Synthesis of Compound 7

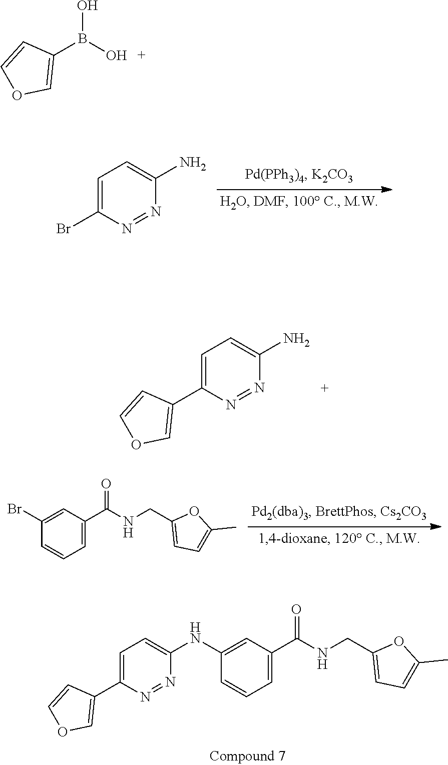

Step 1: Furan-3-ylboronic acid (250 mg, 2.23 mmol), 6-bromopyridazin-3-amine (324 mg, 1.86 mmol), Pd(PPh₃)₄ (108 mg, 0.093 mmol), and potassium carbonate (982 mg, 6.9 mmol) were mixed in H₂O/1,4-dioxane (1.6/6.2 mL) and heated in a microwave reactor for 60 minutes at 100° C. The reaction mixture was concentrated and purified by MPLC to give 6-(furan-3-yl)pyridazin-3-amine (265 mg, 88%) as a yellow solid.

Step 2: 6-(Furan-3-yl)pyridazin-3-amine (20 mg, 0.12 mmol), 3-bromo-N-((5-methylfuran-2-yl)methyl)benzamide (30 mg, 0.103 mmol), Pd₂(dba)₃ (1.9 mg, 0.002 mmol), BrettPhos (11 mg, 0.02 mmol), and cesium carbonate (67 mg, 0.21 mmol) were mixed in 1,4-dioxane (0.5 mL) and heated in a microwave reactor for 60 minutes at 120° C. The reaction mixture was concentrated and purified by MPLC to give compound 7, 3-{[6-(furan-3-yl)pyridazin-3-yl]amino}-N-[(5-methylfuran-2-yl)methyl]benzamide (13 mg, 34%) as a yellowish white solid.

Synthesis of Compound 8

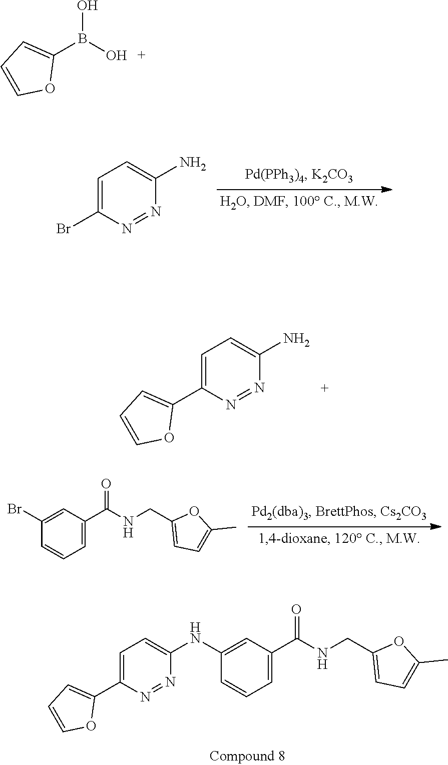

Step 1: Furan-2-ylboronic acid (250 mg, 2.23 mmol), 6-bromopyridazin-3-amine (324 mg, 1.86 mmol), Pd(PPh₃)₄ (108 mg, 0.093 mmol), and potassium carbonate (952 mg, 6.9 mmol) were mixed in H₂O/1,4-dioxane (1.6/6.2 mL) and heated in a microwave reactor for 60 minutes at 100° C. The reaction mixture was concentrated and purified by MPLC to give 6-(furan-2-yl)pyridazin-3-amine (216 mg, 72%) as a yellow solid.

Step 2: 6-(Furan-2-yl)pyridazin-3-amine (20 mg, 0.12 mmol), 3-bromo-N-((5-methylfuran-2-yl)methyl)benzamide (30 mg, 0.103 mmol), Pd₂(dba)₃ (1.9 mg, 0.002 mmol), BrettPhos (11 mg, 0.02 mmol), and cesium carbonate (67 mg, 0.21 mmol) were mixed in 1,4-dioxane (0.5 mL) and heated in a microwave reactor for 60 minutes at 120° C. The reaction mixture was concentrated and purified by MPLC to give compound 8, 3-{[6-(furan-2-yl)pyridazin-3-yl]amino}-N-[(5-methylfuran-2-yl)methyl]benzamide (17 mg, 45%) as a yellowish white solid.

Synthesis of Compound 9

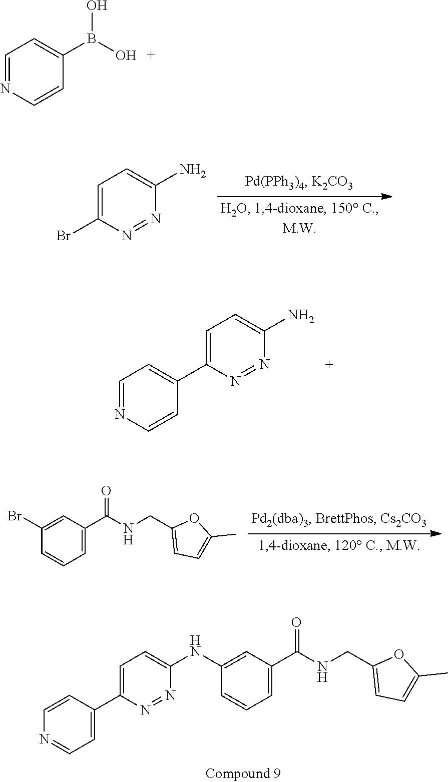

Step 1: Pyridin-4-ylboronic acid (600 mg, 4.9 mmol), 6-bromopyridazin-3-amine (354 mg, 2.03 mmol), Pd(PPh₃)₄ (227 mg, 0.2 mmol), and potassium carbonate (1 g, 7.5 mmol) were mixed in H₂O/1,4-dioxane (1.7/6.8 mL) and heated in a microwave reactor for 90 minutes at 150° C. The reaction mixture was concentrated and purified by MPLC to give 6-(pyridin-4-yl)pyridazin-3-amine (63 mg, 18%) as a yellowish white solid.

Step 2: 6-(Pyridin-4-yl)pyridazin-3-amine (20 mg, 0.12 mmol), 3-bromo-N-((5-methylfuran-2-yl)methyl)benzamide (29 mg, 0.097 mmol), Pd₂(dba)₃ (1.8 mg, 0.0019 mmol), BrettPhos (10.4 mg, 0.019 mmol), and cesium carbonate (63 mg, 0.19 mmol) were mixed in 1,4-dioxane (0.5 mL) and heated in a microwave reactor for 90 minutes at 120° C. The reaction mixture was concentrated and purified by MPLC to give compound 9, N-[(5-methylfuran-2-yl)methyl]-3-{[6-(pyridin-4-yl)pyridazin-3-yl]amino}benzamide (18 mg, 48%) as an orange solid.

Synthesis of Compound 10

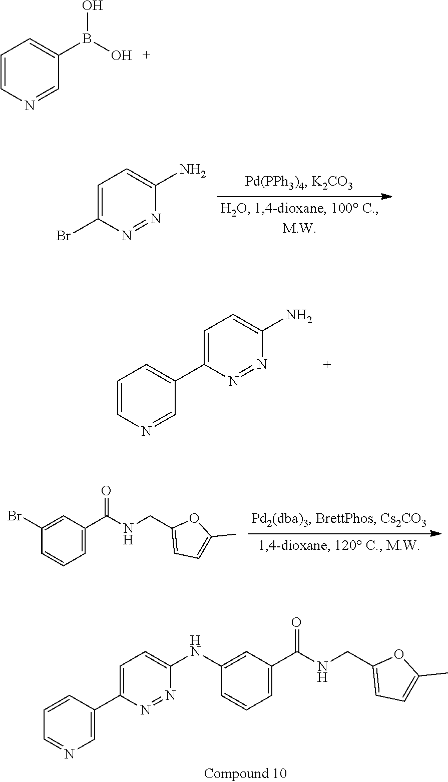

Step 1: Pyridin-3-ylboronic acid (250 mg, 2.03 mmol), 6-bromopyridazin-3-amine (295 mg, 1.7 mmol), Pd(PPh₃)₄ (98 mg, 0.085 mmol), and potassium carbonate (867 mg, 6.3 mmol) were mixed in H₂O/1,4-dioxane (1.4/5.6 mL) and heated in a microwave reactor for 60 minutes at 100° C. The reaction mixture was concentrated and purified by MPLC to give 6-(pyridin-3-yl)pyridazin-3-amine (65 mg, 22%) as a yellowish white solid.

Step 2: 6-(Pyridin-3-yl)pyridazin-3-amine (20 mg, 0.12 mmol), 3-bromo-N-((5-methylfuran-2-yl)methyl)benzamide (29 mg, 0.097 mmol), Pd₂(dba)₃ (1.8 mg, 0.0019 mmol), BrettPhos (10.4 mg, 0.019 mmol), and cesium carbonate (63 mg, 0.19 mmol) were mixed in 1,4-dioxane (0.5 mL) and heated in a microwave reactor for 90 minutes at 120° C. The reaction mixture was concentrated and purified by MPLC. The crude mixture was solidified by using EA to give compound 10, N-[(5-methylfuran-2-yl)methyl]-3-{[6-(pyridin-3-yl)pyridazin-3-yl]amino}benzamide (15 mg, 40%) as a pink solid.

Synthesis of Compound 11

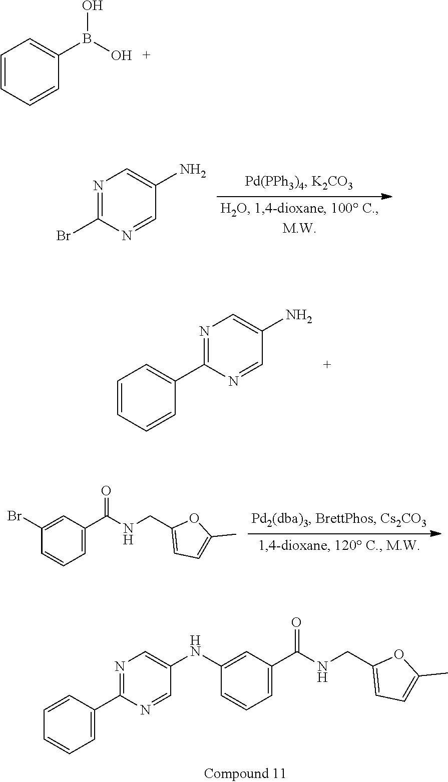

Step 1: Phenylboronic acid (250 mg, 2.05 mmol), 2-bromopyrimidin-5-amine (297 mg, 1.71 mmol), Pd(PPh₃)₄ (99 mg, 0.085 mmol), and potassium carbonate (874 mg, 6.3 mmol) were mixed in H₂O/1,4-dioxane (1.4/5.7 mL) and heated in a microwave reactor for 60 minutes at 120° C. The reaction mixture was concentrated and purified by MPLC to give 2-phenylpyrimidin-5-amine (100 mg, 34%) as a beige solid.

Step 2: 2-Phenylpyrimidin-5-amine (20 mg, 0.12 mmol), 3-bromo-N-((5-methylfuran-2-yl)methyl)benzamide (29 mg, 0.098 mmol), Pd₂(dba)₃ (1.8 mg, 0.002 mmol), BrettPhos (10.5 mg, 0.02 mmol), and cesium carbonate (64 mg, 0.2 mmol) were mixed in 1,4-dioxane (1 mL) and heated in a microwave reactor for 90 minutes at 120° C. The reaction mixture was concentrated and purified by MPLC. The crude mixture was solidified by using EA and HEX to give compound 11, N-[(5-methylfuran-2-yl)methyl]-3-[(2-phenylpyrimidin-5-yl)amino]benzamide (12 mg, 32%) as a beige solid.

Synthesis of Compound 12

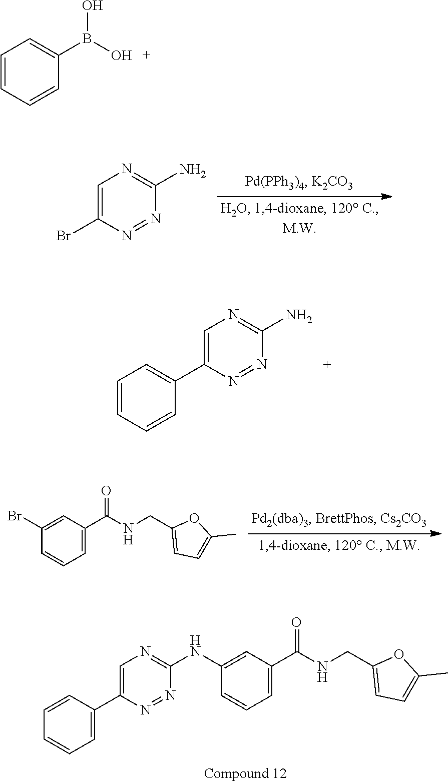

Step 1: Phenylboronic acid (300 mg, 2.5 mmol), 6-bromo-1,2,4-triazin-3-amine (359 mg, 2.05 mmol), Pd(PPh₃)₄ (119 mg, 0.103 mmol), and potassium carbonate (1 g, 7.59 mmol) were mixed in H₂O/1,4-dioxane (1.7/6.8 mL) and heated in a microwave reactor for 60 minutes at 120° C. The reaction mixture was concentrated and purified by MPLC to give 6-phenyl-1,2,4-triazin-3-amine (269 mg, 76%) as a yellowish white solid.

Step 2: 6-Phenyl-1,2,4-triazin-3-amine (20 mg, 0.12 mmol), 3-bromo-N-((5-methylfuran-2-yl)methyl)benzamide (29 mg, 0.098 mmol), Pd₂(dba)₃ (1.8 mg, 0.002 mmol), BrettPhos (10.5 mg, 0.02 mmol), and cesium carbonate (64 mg, 0.2 mmol) were mixed in 1,4-dioxane (0.8 mL) and heated in a microwave reactor for 90 minutes at 120° C. The reaction mixture was concentrated and purified by MPLC. The crude mixture was solidified by using EA to give compound 12, N-[(5-methylfuran-2-yl)methyl]-3-[(6-phenyl-1,2,4-triazin-3-yl)amino]benzamide (12 mg, 32%) as a yellow solid.

Synthesis of Compound 13

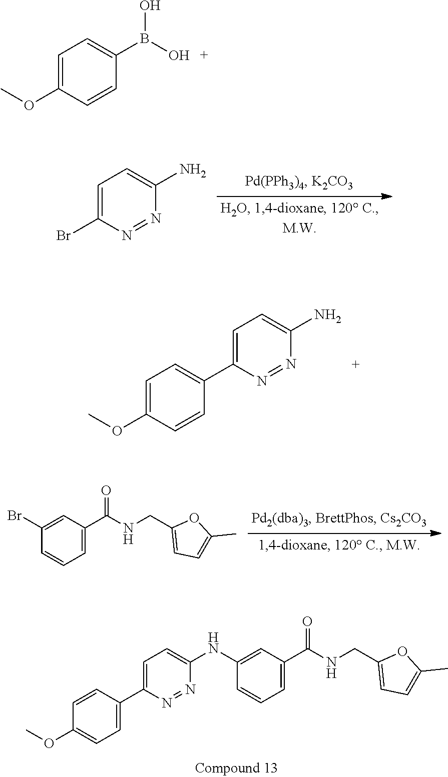

Step 1: (4-Methoxyphenyl)boronic acid (200 mg, 1.3 mmol), 6-bromopyridazin-3-amine (191 mg, 1.1 mmol), Pd(PPh₃)₄ (63 mg, 0.06 mmol), and potassium carbonate (561 mg, 4.06 mmol) were mixed in H₂O/1,4-dioxane (0.9/3.7 mL) and heated in a microwave reactor for 60 minutes at 120° C. The reaction mixture was concentrated and purified by MPLC to give 6-(4-methoxyphenyl)pyridazin-3-amine (189 mg, 86%) as a white solid.

Step 2: 6-(4-Methoxyphenyl)pyridazin-3-amine (20 mg, 0.1 mmol), 3-bromo-N-((5-methylfuran-2-yl)methyl)benzamide (24 mg, 0.08 mmol), Pd₂(dba)₃ (1.5 mg, 0.0017 mmol), BrettPhos (8.9 mg, 0.017 mmol), and cesium carbonate (54 mg, 0.17 mmol) were mixed in 1,4-dioxane (0.4 mL) and heated in a microwave reactor for 90 minutes at 120° C. The reaction mixture was concentrated and purified by MPLC. The crude mixture was solidified by using EA to give compound 13, 3-{[6-(4-methoxyphenyl)pyridazin-3-yl]amino}-N-[(5-methylfuran-2-yl)methyl]benzamide (16 mg, 47%) as a white solid.

Synthesis of Compound 15

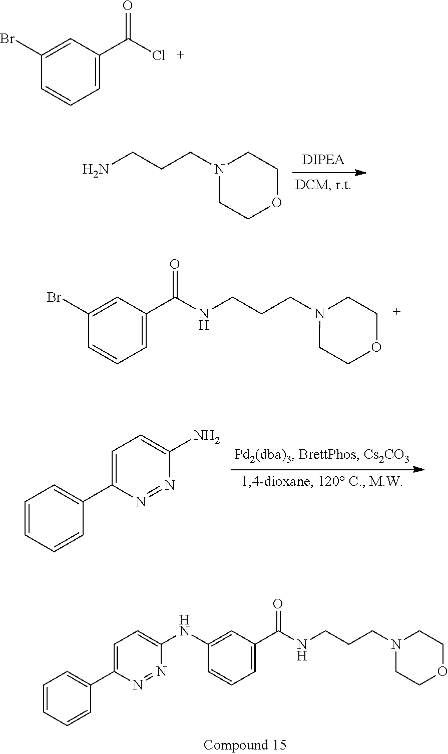

Step 1: 3-Bromobenzoyl chloride (0.12 mL, 0.91 mmol) and 3-morpholinopropan-1-amine (0.11 mL, 0.76 mmol) were dissolved in DCM (7.6 mL), followed up by addition of DIPEA (0.28 mL, 1.63 mmol) and stirred for 25 hours at room temperature. The reaction mixture was concentrated and purified by MPLC. And the mixture was extracted by EA and brine. The organic layer was dried over anhydrous Na₂SO₄ and concentrated to give 3-bromo-N-(3-morpholinopropyl)benzamide (338 mg, >99%) as a brownish oil.

Step 2: 6-Phenylpyridazin-3-amine (20 mg, 0.12 mmol), 3-bromo-N-(3-morpholinopropyl)benzamide (32 mg, 0.097 mmol), Pd₂(dba)₃ (8.9 mg, 0.0097 mmol), BrettPhos (10.5 mg, 0.019 mmol), and cesium carbonate (63 mg, 0.19 mmol) were mixed in 1,4-dioxane (0.5 mL) and heated in a microwave reactor for 90 minutes at 120° C. The reaction mixture was concentrated and purified by MPLC. The crude mixture was solidified by using EA to give compound 15, N-[3-(morpholin-4-yl)propyl]-3-[(6-phenylpyridazin-3-yl)amino]benzamide (9 mg, 23%) as a beige solid.

Synthesis of Compound 16

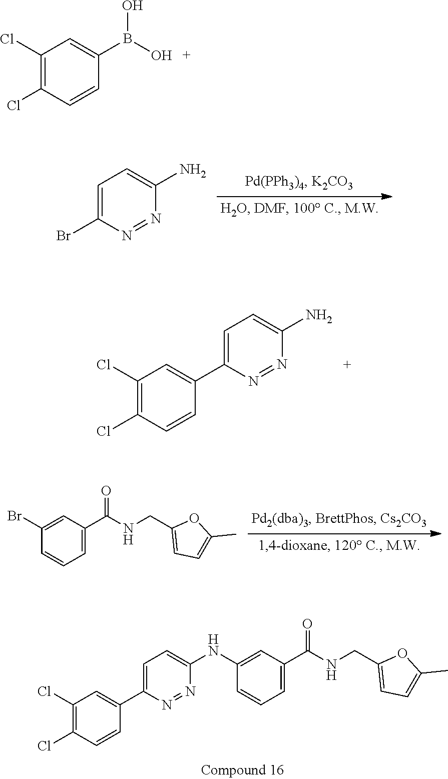

Step 1: (3,4-Dichlorophenyl)boronic acid (200 mg, 1.05 mmol), 6-bromopyridazin-3-amine (152 mg, 0.87 mmol), Pd(PPh₃)₄ (51 mg, 0.04 mmol), and potassium carbonate (447 mg, 3.23 mmol) were mixed in H₂O/1,4-dioxane (0.7/2.9 mL) and heated in a microwave reactor for 60 minutes at 120° C. The reaction mixture was concentrated and purified by MPLC to give 6-(3,4-dichlorophenyl)pyridazin-3-amine (62 mg, 29%) as a yellowish white solid.

Step 2: 6-(3,4-Dichlorophenyl)pyridazin-3-amine (20 mg, 0.083 mmol), 3-bromo-N-((5-methylfuran-2-yl)methyl)benzamide (20 mg, 0.07 mmol), Pd₂(dba)₃ (6.4 mg, 0.0069 mmol), BrettPhos (7.5 mg, 0.014 mmol), and cesium carbonate (45 mg, 0.14 mmol) were mixed in 1,4-dioxane (0.35 mL) and heated in a microwave reactor for 90 minutes at 120° C. The reaction mixture was concentrated and purified by MPLC. The crude mixture was solidified by using EA to give compound 16, 3-{[6-(3,4-dichlorophenyl)pyridazin-3-yl]amino}-N-[(5-methylfuran-2-yl)methyl]benzamide (3 mg, 10%) as a beige solid.

Synthesis of Compound 18

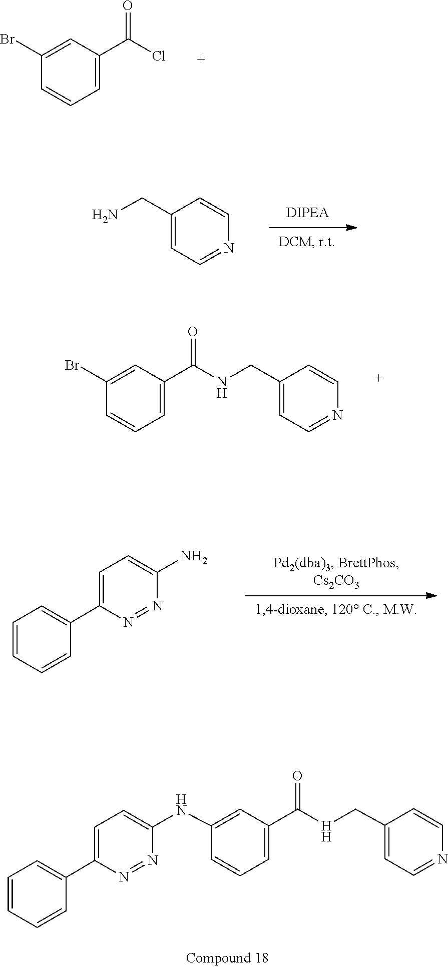

Step 1: 3-Bromobenzoyl chloride (0.12 mL, 0.91 mmol) and pyridin-4-ylmethanamine (0.08 mL, 0.76 mmol) were dissolved in DCM (7.6 mL), followed up by addition of DIPEA (0.28 mL, 1.6 mmol) and stirred for 28 hours at room temperature. The reaction mixture was extracted by EA and saturated aq. NH₄Cl. The organic layer was dried over anhydrous Na₂SO₄ and concentrated to give 3-bromo-N-(pyridin-4-ylmethyl)benzamide (246 mg, >99%) as a brown oil.

Step 2: 6-Phenylpyridazin-3-amine (30 mg, 0.18 mmol), 3-bromo-N-(pyridin-4-ylmethyl)benzamide (46 mg, 0.16 mmol), Pd₂(dba)₃ (14.6 mg, 0.016 mmol), BrettPhos (17 mg, 0.032 mmol), and cesium carbonate (104 mg, 0.32 mmol) were mixed in 1,4-dioxane (1 mL) and heated in a microwave reactor for 90 minutes at 120° C. The reaction mixture was extracted by MeOH/DCM (10:1) and H₂O. The crude mixture was solidified by using DCM to give compound 18, 3-[(6-phenylpyridazin-3-yl)amino]-N-[(pyridin-4-yl)methyl]benzamide (17 mg, 28%) as a yellow solid.

Synthesis of Compound 19

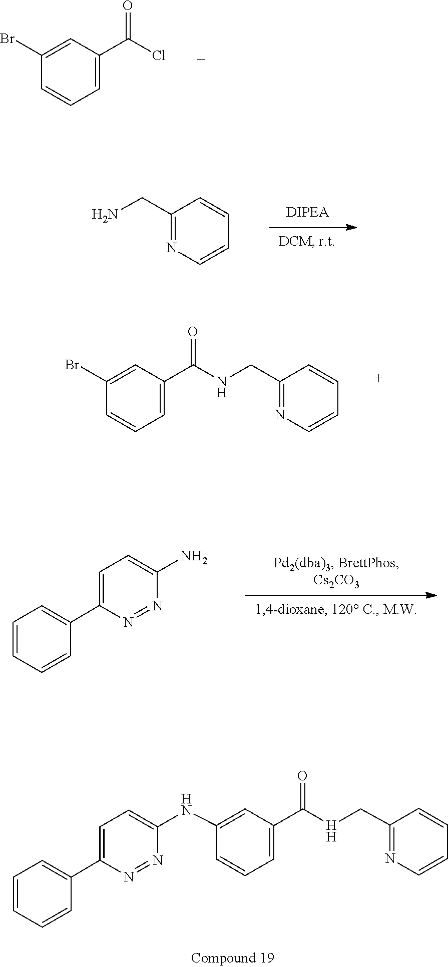

Step 1: 3-Bromobenzoyl chloride (0.12 mL, 0.91 mmol) and pyridin-2-ylmethanamine (0.08 mL, 0.76 mmol) were dissolved in DCM (7.6 mL), followed up by addition of DIPEA (0.28 mL, 1.63 mmol) and stirred for 28 hours at room temperature. The reaction mixture was extracted by EA and saturated aq. NH₄Cl. The organic layer was dried over anhydrous Na₂SO₄ and concentrated to give 3-bromo-N-(pyridin-2-ylmethyl)benzamide (268 mg, >99%) as a brown oil.

Step 2: 6-Phenylpyridazin-3-amine (30 mg, 0.18 mmol), 3-bromo-N-(pyridin-2-ylmethyl)benzamide (46 mg, 0.16 mmol), Pd₂(dba)₃ (14.6 mg, 0.016 mmol), BrettPhos (17 mg, 0.032 mmol), and cesium carbonate (104 mg, 0.32 mmol) were mixed in 1,4-dioxane (1 mL) and heated in a microwave reactor for 90 minutes at 120° C. The reaction mixture was concentrated and purified by MPLC to give compound 19, 3-[(6-phenylpyridazin-3-yl)amino]-N-[(pyridin-2-yl)methyl]benzamide (39 mg, 65%) as a yellowish white solid.

Synthesis of Compound 20

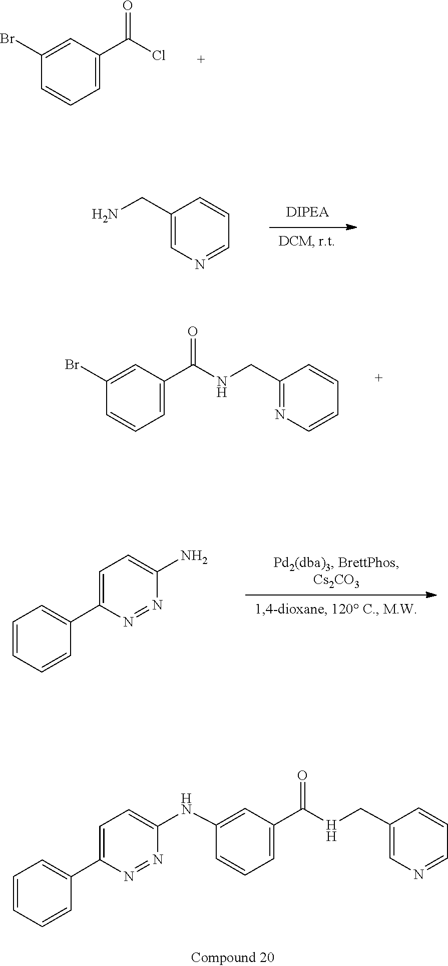

Step 1: 3-Bromobenzoyl chloride (0.12 mL, 0.91 mmol) and pyridin-3-ylmethanamine (0.08 mL, 0.76 mmol) were dissolved in DCM (7.6 mL), followed up by addition of DIPEA (0.28 mL, 1.63 mmol) and stirred for 26 hours at room temperature. The reaction mixture was extracted by EA and saturated aq. NH₄Cl. The organic layer was dried over anhydrous Na₂SO₄ and concentrated to give 3-bromo-N-(pyridin-3-ylmethyl)benzamide (268 mg, >99%) as a brown oil.

Step 2: 6-Phenylpyridazin-3-amine (30 mg, 0.18 mmol), 3-bromo-N-(pyridin-3-ylmethyl)benzamide (46 mg, 0.16 mmol), Pd₂(dba)₃ (14.6 mg, 0.016 mmol), BrettPhos (17 mg, 0.032 mmol), and cesium carbonate (104 mg, 0.32 mmol) were mixed in 1,4-dioxane (1 mL) and heated in a microwave reactor for 90 minutes at 120° C. The reaction mixture was concentrated and purified by MPLC. The crude mixture was solidified by using EA to give compound 20, 3-[(6-phenylpyridazin-3-yl)amino]-N-[(pyridin-3-yl)methyl]benzamide (41 mg, 60%) as a beige solid.

Synthesis of Compound 21

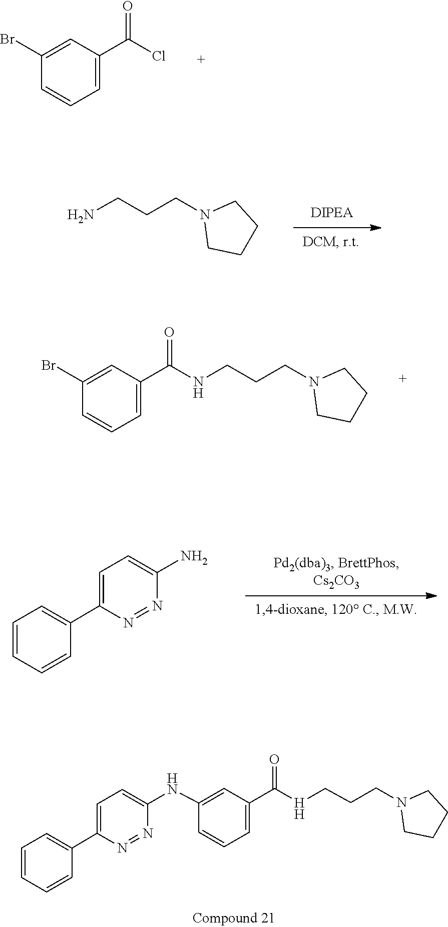

Step 1: 3-Bromobenzoyl chloride (0.12 mL, 0.91 mmol) and 3-(pyrrolidin-1-yl)propan-1-amine (0.1 mL, 0.76 mmol) were dissolved in DCM (7.6 mL), followed up by addition of DIPEA (0.28 mL, 1.63 mmol) and stirred for 26 hours at room temperature. The reaction mixture was extracted by EA and saturated aq. NH₄Cl. The organic layer was dried over anhydrous Na₂SO₄. The mixture (3-bromo-N-(3-(pyrrolidin-1-yl)propyl)benzamide) was concentrated and used in the next step without further purification.

Step 2: 6-Phenylpyridazin-3-amine (40 mg, 0.23 mmol), 3-bromo-N-(3-(pyrrolidin-1-yl)propyl)benzamide (121 mg, 0.19 mmol), Pd₂(dba)₃ (18 mg, 0.019 mmol), BrettPhos (21 mg, 0.039 mmol), and cesium carbonate (127 mg, 0.39 mmol) were mixed in 1,4-dioxane (1 mL) and heated in a microwave reactor for 90 minutes at 120° C. The reaction mixture was extracted by EA and saturated aq. NH₄Cl. The organic layer was dried over anhydrous Na₂SO₄ and concentrated. The residue was purified by preparative thin layer chromatography (PTLC). The crude mixture was solidified by using EA to give compound 21, 3-[(6-phenylpyridazin-3-yl)amino]-N-[3-(pyrrolidin-1-yl)propyl]benzamide (15 mg, 19%) as a beige solid.

Synthesis of Compound 22

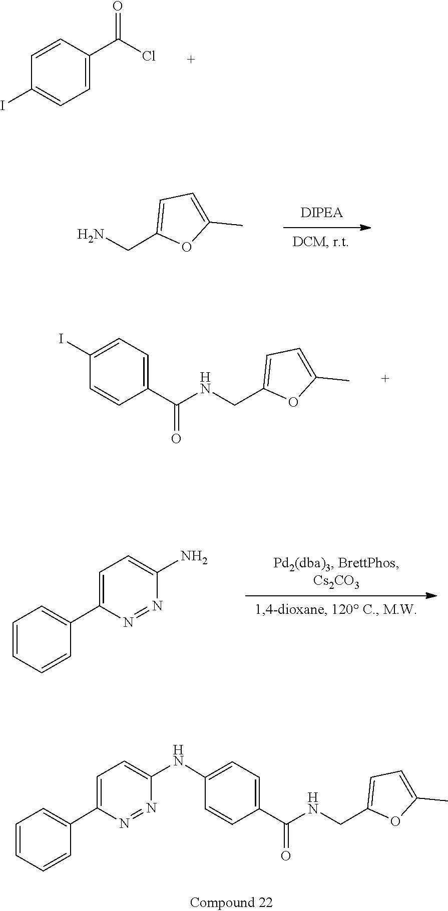

Step 1: 4-Iodobenzoyl chloride (288 mg, 1.08 mmol) and (5-methylfuran-2-yl)methanamine (0.98 mL, 0.9 mmol) were dissolved in DCM (9 mL), followed up by addition of DIPEA (0.34 mL, 1.9 mmol) and stirred for 22 hours at room temperature. The reaction mixture was extracted by EA and saturated aq. NH₄Cl. The organic layer was dried over anhydrous Na₂SO₄ and concentrated. The residue was purified by MPLC to give 4-iodo-N-((5-methylfuran-2-yl)methyl)benzamide (295 mg, 96%) as a beige solid.

Step 2: 6-Phenylpyridazin-3-amine (100 mg, 0.58 mmol), 4-iodo-N-((5-methylfuran-2-yl)methyl)benzamide (219 mg, 0.64 mmol), Pd₂(dba)₃ (53 mg, 0.058 mmol), BrettPhos (63 mg, 0.12 mmol), and cesium carbonate (381 mg, 1.17 mmol) were mixed in 1,4-dioxane (4 mL) and heated in a microwave reactor for 90 minutes at 120° C. The reaction mixture was concentrated and purified by MPLC. The crude mixture was solidified by using MeOH/DCM (1:10) to give compound 22, N-[(5-methylfuran-2-yl)methyl]-4-[(6-phenylpyridazin-3-yl)amino]benzamide (21 mg, 9%) as a white solid.

Synthesis of Compound 23

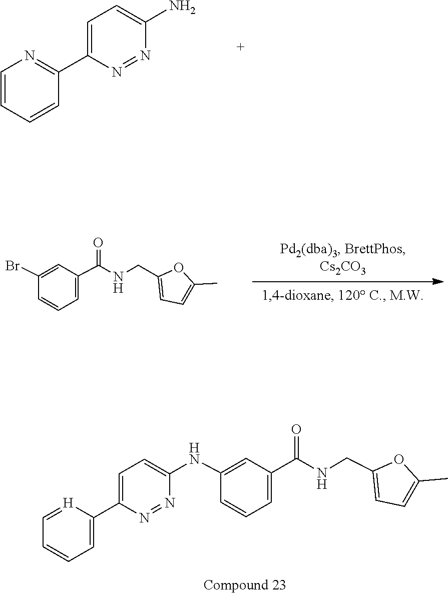

6-(Pyridin-2-yl)pyridazin-3-amine (30 mg, 0.17 mmol), 3-bromo-N-((5-methylfuran-2-yl)methyl)benzamide (46 mg, 0.16 mmol), Pd₂(dba)₃ (14 mg, 0.016 mmol), BrettPhos (17 mg, 0.03 mmol), and cesium carbonate (103 mg, 0.32 mmol) were mixed in 1,4-dioxane (1.1 mL) and heated in a microwave reactor for 90 minutes at 120° C. The reaction mixture was extracted by MeOH/DCM (1:10) and brine. The organic layer was dried over anhydrous Na₂SO₄ and concentrated. The residue was purified by MPLC. The crude mixture was solidified by using DCM and EA to give compound 23, N-[(5-methylfuran-2-yl)methyl]-3-{[6-(pyridin-2-yl)pyridazin-3-yl]amino}benzamide (8 mg, 13%) as a beige solid.

Synthesis of Compound 24

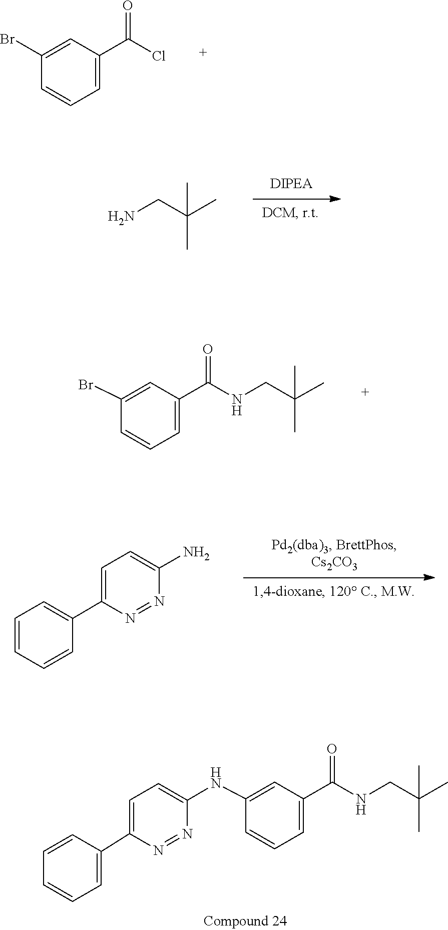

Step 1: 3-Bromobenzoyl chloride (0.12 mL, 0.91 mmol) and 2,2-dimethylpropan-1-amine (0.08 mL, 0.76 mmol) were dissolved in DCM (7.6 mL), followed up by addition of DIPEA (0.28 mL, 1.63 mmol) and stirred for 26 hours at room temperature. The reaction mixture was concentrated and purified by MPLC to give 3-bromo-N-neopentylbenzamide (166 mg, 66%) as a white solid.

Step 2: 6-Phenylpyridazin-3-amine (40 mg, 0.23 mmol), 3-bromo-N-neopentylbenzamide (63 mg, 0.23 mmol), Pd₂(dba)₃ (21 mg, 0.023 mmol), BrettPhos (25 mg, 0.046 mmol), and cesium carbonate (151 mg, 0.46 mmol) were mixed in 1,4-dioxane (1.1 mL) and heated in a microwave reactor for 90 minutes at 120° C. The reaction mixture was concentrated and purified by MPLC. The crude mixture was solidified by using EA to give compound 24, N-(2,2-dimethylpropyl)-3-[(6-phenylpyridazin-3-yl)amino]benzamide (20 mg, 24%) as a beige solid.

Synthesis of Compound 25

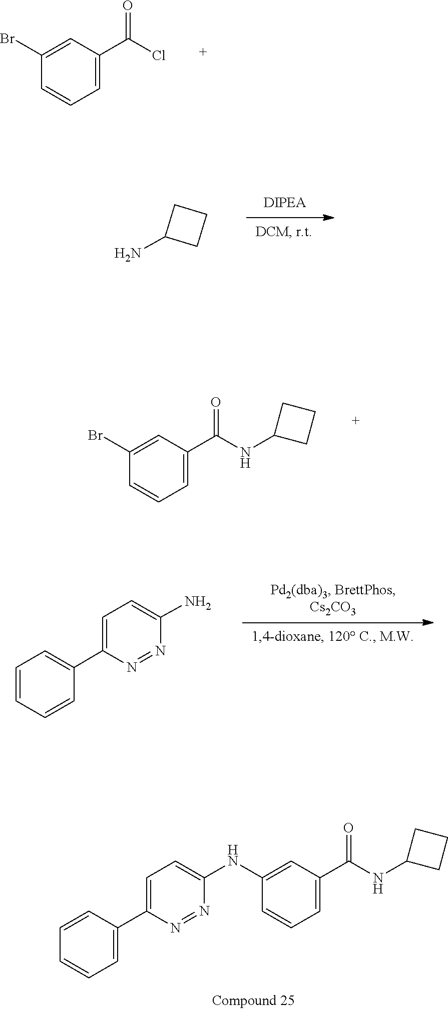 [SMILES: O=C(Cl)c1cccc(Br)c1 ; NC1CCC1 ; O=C(NC1CCC1)c1cccc(Br)c1 ; Nc1ccc(-c2ccccc2)nn1 ; O=C(NC1CCC1)c1cccc(Nc2ccc(-c3ccccc3)nn2)c1]

Step 1: 3-Bromobenzoyl chloride (0.12 mL, 0.91 mmol) and cyclobutanamine (54 mg, 0.76 mmol) were dissolved in DCM (7.6 mL), followed up by addition of DIPEA (0.28 mL, 1.63 mmol) and stirred for 25 hours at room temperature. The reaction mixture was concentrated and purified by MPLC to give 3-bromo-N-cyclobutylbenzamide (201 mg, >99%) as a white solid.

Step 2: 6-Phenylpyridazin-3-amine (40 mg, 0.23 mmol), 3-bromo-N-cyclobutylbenzamide (54 mg, 0.21 mmol), Pd₂(dba)₃ (20 mg, 0.021 mmol), BrettPhos (23 mg, 0.042 mmol), and cesium carbonate (138 mg, 0.42 mmol) were mixed in 1,4-dioxane (1.1 mL) and heated in a microwave reactor for 90 minutes at 120° C. The reaction mixture was concentrated and purified by MPLC. The crude mixture was solidified by using DCM to give compound 25, N-cyclobutyl-3-[(6-phenylpyridazin-3-yl)amino]benzamide (20 mg, 27%) as a white solid.

Synthesis of Compound 26

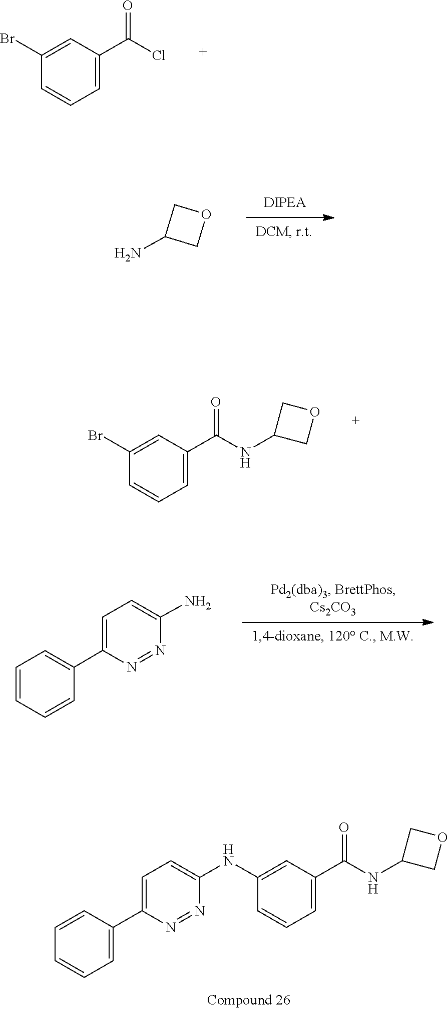 [SMILES: O=C(Cl)c1cccc(Br)c1 ; NC1COC1 ; O=C(NC1COC1)c1cccc(Br)c1 ; Nc1ccc(-c2ccccc2)nn1 ; O=C(NC1COC1)c1cccc(Nc2ccc(-c3ccccc3)nn2)c1]

Step 1: 3-Bromobenzoyl chloride (0.12 mL, 0.91 mmol) and oxetan-3-amine (56 mg, 0.76 mmol) were dissolved in DCM (7.6 mL), followed up by addition of DIPEA (0.28 mL, 1.63 mmol) and stirred for 23 hours at room temperature. The reaction mixture was concentrated and purified by MPLC to give 3-bromo-N-(oxetan-3-yl)benzamide (197 mg, >99%) as a white solid.

Step 2: 6-Phenylpyridazin-3-amine (40 mg, 0.23 mmol), 3-bromo-N-(oxetan-3-yl)benzamide (54 mg, 0.21 mmol), Pd₂(dba)₃ (20 mg, 0.021 mmol), BrettPhos (23 mg, 0.042 mmol), and cesium carbonate (138 mg, 0.42 mmol) were mixed in 1,4-dioxane (1.1 mL) and heated in a microwave reactor for 90 minutes at 120° C. The reaction mixture was concentrated and purified by MPLC. The crude mixture was solidified by using EA to give compound 26, N-(oxetan-3-yl)-3-[(6-phenylpyridazin-3-yl)amino]benzamide (14 mg, 19%) as a beige solid.

Synthesis of Compound 27

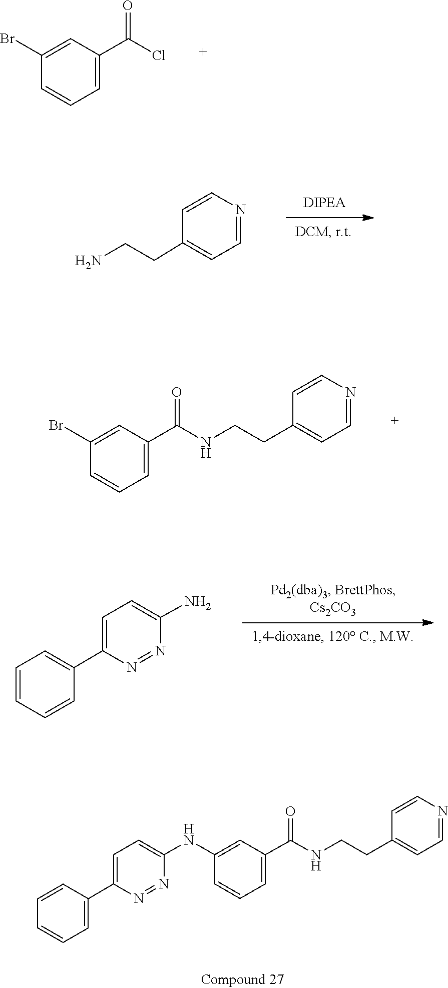 [SMILES: O=C(Cl)c1cccc(Br)c1 ; NCCc1ccncc1 ; O=C(NCCc1ccncc1)c1cccc(Br)c1 ; Nc1ccc(-c2ccccc2)nn1 ; O=C(NCCc1ccncc1)c1cccc(Nc2ccc(-c3ccccc3)nn2)c1]

Step 1: 3-Bromobenzoyl chloride (0.12 mL, 0.91 mmol) and 2-(pyridin-4-yl)ethan-1-amine (93 mg, 0.76 mmol) were dissolved in DCM (7.6 mL), followed up by addition of DIPEA (0.28 mL, 1.63 mmol) and stirred for 26 hours at room temperature. The reaction mixture was extracted by EA and saturated aq. NH₄Cl. The organic layer was dried over anhydrous Na₂SO₄ and concentrated. The residue was purified by MPLC to give 3-bromo-N-(2-(pyridin-4-yl)ethyl)benzamide (170 mg, 73%) as a beige solid.

Step 2: 6-Phenylpyridazin-3-amine (40 mg, 0.23 mmol), 3-bromo-N-(2-(pyridin-4-yl)ethyl)benzamide (65 mg, 0.21 mmol), Pd₂(dba)₃ (20 mg, 0.021 mmol), BrettPhos (23 mg, 0.042 mmol), and cesium carbonate (138 mg, 0.42 mmol) were mixed in 1,4-dioxane (1.4 mL) and heated in a microwave reactor for 90 minutes at 120° C. The reaction mixture was concentrated and purified by MPLC to give compound 27, 3-[(6-phenylpyridazin-3-yl)amino]-N-[2-(pyridin-4-yl)ethyl]benzamide (49 mg, 83%) as a beige solid.

Synthesis of Compound 28

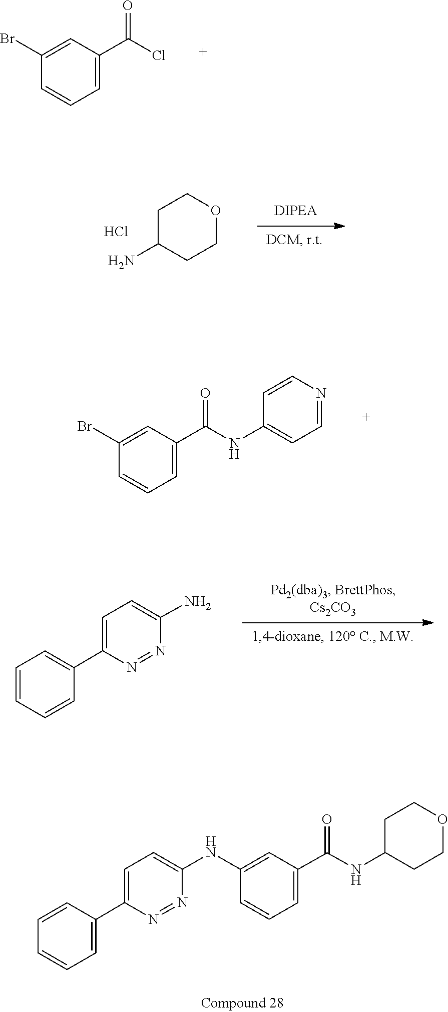 [SMILES: O=C(Cl)c1cccc(Br)c1 ; Cl ; NC1CCOCC1 ; O=C(Nc1ccncc1)c1cccc(Br)c1 ; Nc1ccc(-c2ccccc2)nn1 ; O=C(NC1CCOCC1)c1cccc(Nc2ccc(-c3ccccc3)nn2)c1]

Step 1: 3-Bromobenzoyl chloride (200 mg, 0.91 mmol) and tetrahydro-2H-pyran-4-amine hydrochloride (104 mg, 0.76 mmol) were dissolved in DCM (7.6 mL), followed up by addition of DIPEA (0.42 mL, 2.4 mmol) and stirred for 20 hours at room temperature. The reaction mixture was concentrated and purified by MPLC to give 3-bromo-N-(tetrahydro-2H-pyran-4-yl)benzamide (206 mg, 96%) as a white solid.

Step 2: 6-Phenylpyridazin-3-amine (40 mg, 0.23 mmol), 3-bromo-N-(tetrahydro-2H-pyran-4-yl)benzamide (60 mg, 0.21 mmol), Pd₂(dba)₃ (20 mg, 0.021 mmol), BrettPhos (23 mg, 0.042 mmol), and cesium carbonate (138 mg, 0.42 mmol) were mixed in 1,4-dioxane (1.1 mL) and heated in a microwave reactor for 90 minutes at 120° C. The reaction mixture was concentrated and purified by MPLC. The crude mixture was solidified by using EA and DCM to give compound 28, N-(oxan-4-yl)-3-[(6-phenylpyridazin-3-yl)amino]benzamide (31 mg, 39%) as a white solid.

Synthesis of Compound 29

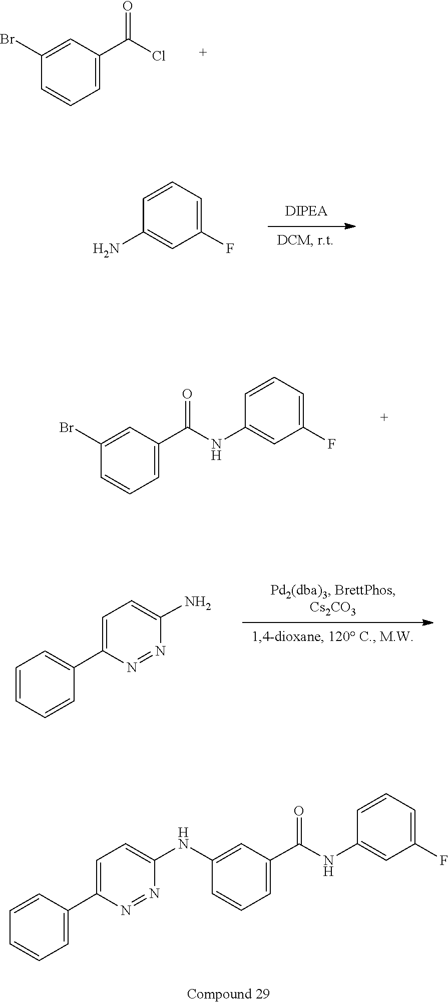 [SMILES: O=C(Cl)c1cccc(Br)c1 ; Nc1cccc(F)c1 ; O=C(Nc1cccc(F)c1)c1cccc(Br)c1 ; Nc1ccc(-c2ccccc2)nn1 ; O=C(Nc1cccc(F)c1)c1cccc(Nc2ccc(-c3ccccc3)nn2)c1]

Step 1: 3-Bromobenzoyl chloride (0.12 mL, 0.91 mmol) and 3-fluoroaniline (84 mg, 0.76 mmol) were dissolved in DCM (7.6 mL), followed up by addition of DIPEA (0.28 mL, 1.63 mmol) and stirred for 22 hours at room temperature. The reaction mixture was concentrated and purified by MPLC to give 3-bromo-N-(3-fluorophenyl)benzamide (223 mg, >99%) as a white solid.

Step 2: 6-Phenylpyridazin-3-amine (40 mg, 0.23 mmol), 3-bromo-N-(3-fluorophenyl)benzamide (62 mg, 0.21 mmol), Pd₂(dba)₃ (20 mg, 0.021 mmol), BrettPhos (23 mg, 0.042 mmol), and cesium carbonate (138 mg, 0.42 mmol) were mixed in 1,4-dioxane (1.1 mL) and heated in a microwave reactor for 90 minutes at 120° C. The reaction mixture was concentrated and purified by MPLC. The crude mixture was solidified by using EA to give compound 29, N-(3-fluorophenyl)-3-[(6-phenylpyridazin-3-yl)amino]benzamide (25 mg, 30%) as a white solid.

Synthesis of Compound 30

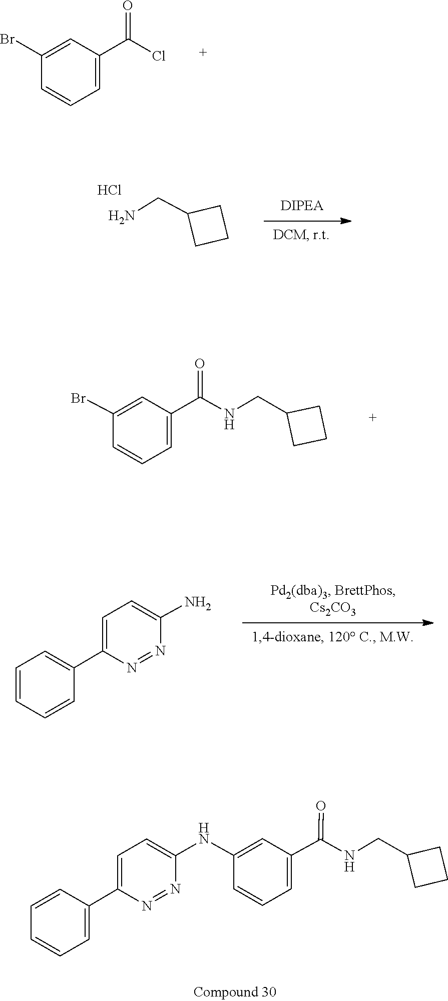 [SMILES: O=C(Cl)c1cccc(Br)c1 ; Cl ; NCC1CCC1 ; O=C(NCC1CCC1)c1cccc(Br)c1 ; Nc1ccc(-c2ccccc2)nn1 ; O=C(NCC1CCC1)c1cccc(Nc2ccc(-c3ccccc3)nn2)c1]

Step 1: 3-Bromobenzoyl chloride (0.12 mL, 0.91 mmol) and cyclobutylmethanamine hydrochloride (92 mg, 0.76 mmol) were dissolved in DCM (7.6 mL), followed up by addition of DIPEA (0.42 mL, 2.4 mmol) and stirred for 19 hours at room temperature. The reaction mixture was concentrated and purified by MPLC to give 3-bromo-N-(cyclobutylmethyl)benzamide (210 mg, >99%) as a white solid.

Step 2: 6-Phenylpyridazin-3-amine (40 mg, 0.23 mmol), 3-bromo-N-(cyclobutylmethyl)benzamide (57 mg, 0.21 mmol), Pd₂(dba)₃ (20 mg, 0.021 mmol), BrettPhos (23 mg, 0.042 mmol), and cesium carbonate (138 mg, 0.42 mmol) were mixed in 1,4-dioxane (1.1 mL) and heated in a microwave reactor for 90 minutes at 120° C. The reaction mixture was concentrated and purified by MPLC. The crude mixture was solidified by using EA to give compound 30, N-(cyclobutylmethyl)-3-[(6-phenylpyridazin-3-yl)amino]benzamide (24 mg, 31%) as a white solid.

Synthesis of Compound 31

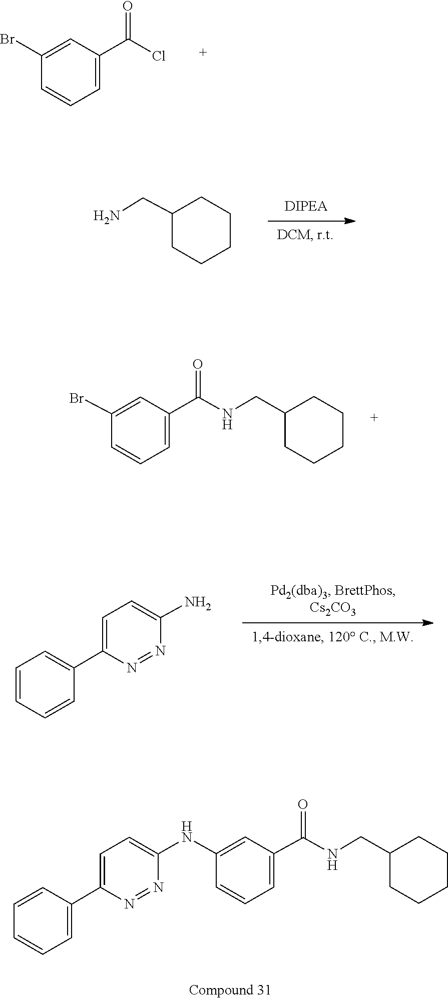 [SMILES: O=C(Cl)c1cccc(Br)c1 ; NCC1CCCCC1 ; O=C(NCC1CCCCC1)c1cccc(Br)c1 ; Nc1ccc(-c2ccccc2)nn1 ; O=C(NCC1CCCCC1)c1cccc(Nc2ccc(-c3ccccc3)nn2)c1]

Step 1: 3-Bromobenzoyl chloride (0.12 mL, 0.91 mmol) and cyclohexylmethanamine (86 mg, 0.76 mmol) were dissolved in DCM (7.6 mL), followed up by addition of DIPEA (0.28 mL, 1.63 mmol) and stirred for 19 hours at room temperature. The reaction mixture was concentrated and purified by MPLC to give 3-bromo-N-(cyclohexylmethyl)benzamide (192 mg, 86%) as a white solid.

Step 2: 6-Phenylpyridazin-3-amine (40 mg, 0.23 mmol), 3-bromo-N-(cyclohexylmethyl)benzamide (63 mg, 0.21 mmol), Pd₂(dba)₃ (20 mg, 0.021 mmol), BrettPhos (23 mg, 0.042 mmol), and cesium carbonate (138 mg, 0.42 mmol) were mixed in 1,4-dioxane (1.1 mL) and heated in a microwave reactor for 90 minutes at 120° C. The reaction mixture was concentrated and purified by MPLC to give compound 31, N-(cyclohexylmethyl)-3-[(6-phenylpyridazin-3-yl)amino]benzamide (16 mg, 19%) as a yellowish white solid.

Synthesis of Compound 32

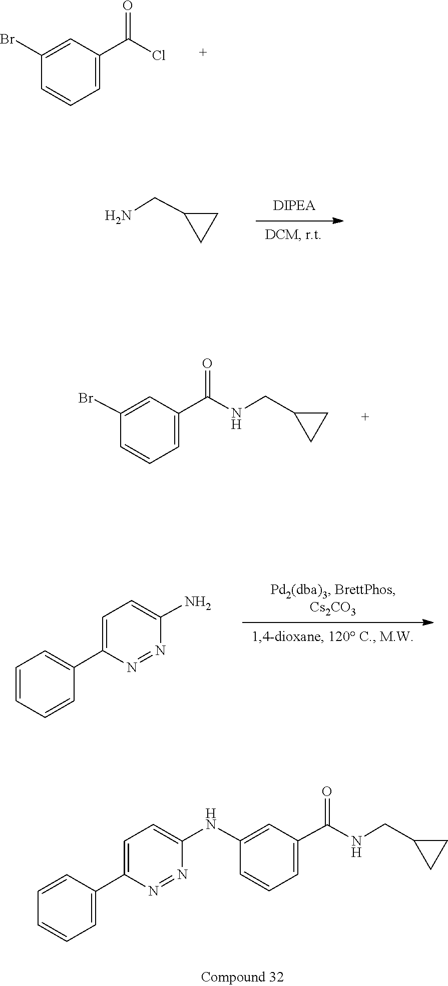 [SMILES: O=C(Cl)c1cccc(Br)c1 ; NCC1CC1 ; O=C(NCC1CC1)c1cccc(Br)c1 ; Nc1ccc(-c2ccccc2)nn1 ; O=C(NCC1CC1)c1cccc(Nc2ccc(-c3ccccc3)nn2)c1]

Step 1: 3-Bromobenzoyl chloride (0.12 mL, 0.91 mmol) and cyclopropylmethanamine (54 mg, 0.76 mmol) were dissolved in DCM (7.6 mL), followed up by addition of DIPEA (0.28 mL, 1.63 mmol) and stirred for 19 hours at room temperature. The reaction mixture was concentrated and purified by MPLC to give 3-bromo-N-(cyclopropylmethyl)benzamide (138 mg, 72%) as a white solid.

Step 2: 6-Phenylpyridazin-3-amine (40 mg, 0.23 mmol), 3-bromo-N-(cyclopropylmethyl)benzamide (59 mg, 0.23 mmol), Pd₂(dba)₃ (21 mg, 0.023 mmol), BrettPhos (25 mg, 0.046 mmol), and cesium carbonate (152 mg, 0.47 mmol) were mixed in 1,4-dioxane (1.2 mL) and heated in a microwave reactor for 90 minutes at 120° C. The reaction mixture was concentrated and purified by MPLC. The crude mixture was solidified by using EA to give compound 32, N-(cyclopropylmethyl)-3-[(6-phenylpyridazin-3-yl)amino]benzamide (22 mg, 27%) as a white solid.

Synthesis of Compound 33

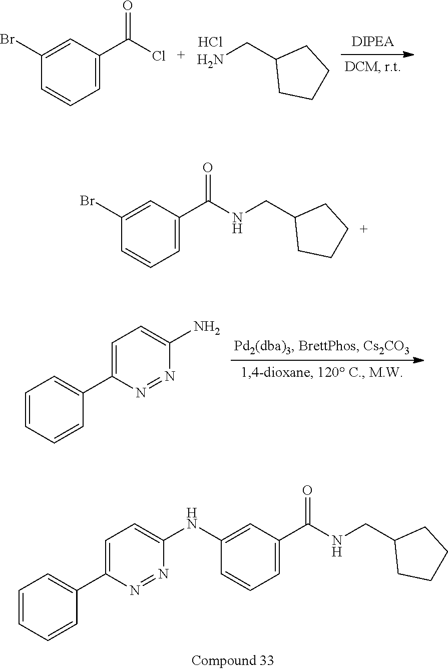

Step 1: 3-Bromobenzoyl chloride (0.12 mL, 0.91 mmol) and cyclopentylmethanamine hydrochloride (103 mg, 0.76 mmol) were dissolved in DCM (7.6 mL), followed up by addition of DIPEA (0.42 mL, 2.4 mmol) and stirred for 19 hours at room temperature. The reaction mixture was concentrated and purified by MPLC to give 3-bromo-N-(cyclopentylmethyl)benzamide (210 mg, 98%) as a white solid.

Step 2: 6-Phenylpyridazin-3-amine (40 mg, 0.23 mmol), 3-bromo-N-(cyclopentylmethyl)benzamide (66 mg, 0.23 mmol), Pd₂(dba)₃ (21 mg, 0.023 mmol), BrettPhos (25 mg, 0.046 mmol), and cesium carbonate (152 mg, 0.47 mmol) were mixed in 1,4-dioxane (1.2 mL) and heated in a microwave reactor for 90 minutes at 120° C. The reaction mixture was concentrated and purified by MPLC to give compound 33, N-(cyclopentylmethyl)-3-[(6-phenylpyridazin-3-yl)amino]benzamide (17 mg, 19%) as a yellowish white solid.

Synthesis of Compound 34

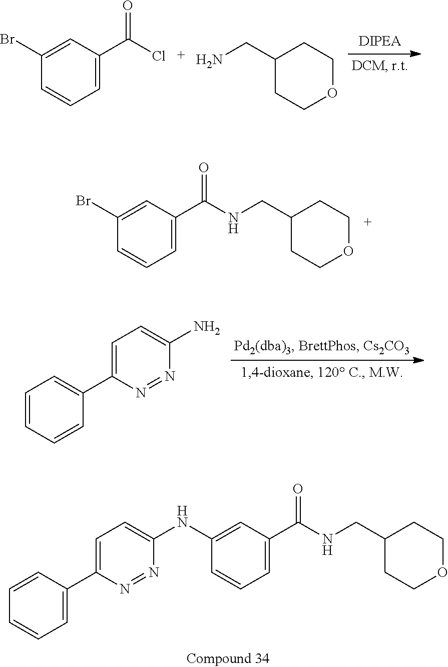

Step 1: 3-Bromobenzoyl chloride (200 mg, 0.91 mmol) and (tetrahydro-2H-pyran-4-yl)methanamine (88 mg, 0.76 mmol) were dissolved in DCM (7.6 mL), followed up by addition of DIPEA (0.28 mL, 1.63 mmol) and stirred for 21 hours at room temperature. The reaction mixture was concentrated and purified by MPLC to give 3-bromo-N-((tetrahydro-2H-pyran-4-yl)methyl)benzamide (163 mg, 72%) as a white solid.

Step 2: 6-Phenylpyridazin-3-amine (49 mg, 0.29 mmol), 3-bromo-N-((tetrahydro-2H-pyran-4-yl)methyl)benzamide (85 mg, 0.29 mmol), Pd₂(dba)₃ (26 mg, 0.03 mmol), BrettPhos (31 mg, 0.06 mmol), and cesium carbonate (186 mg, 0.57 mmol) were mixed in 1,4-dioxane (1.4 mL) and heated in a microwave reactor for 90 minutes at 120° C. The reaction mixture was concentrated and purified by MPLC. The crude mixture was solidified by using HEX and EA to give compound 34, N-[(oxan-4-yl)methyl]-3-[(6-phenylpyridazin-3-yl)amino]benzamide (28 mg, 25%) as a beige solid.

Synthesis of Compound 35

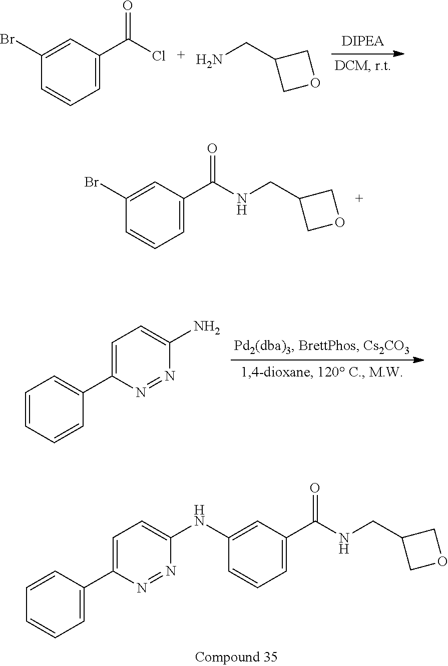

Step 1: 3-Bromobenzoyl chloride (0.12 mL, 0.91 mmol) and oxetan-3-ylmethanamine (66 mg, 0.76 mmol) were dissolved in DCM (7.6 mL), followed up by addition of DIPEA (0.28 mL, 1.63 mmol) and stirred for 23 hours at room temperature. The reaction mixture was concentrated and purified by MPLC to give 3-bromo-N-(oxetan-3-ylmethyl)benzamide (195 mg, 95%) as a yellow oil.

Step 2: 6-Phenylpyridazin-3-amine (40 mg, 0.23 mmol), 3-bromo-N-(oxetan-3-ylmethyl)benzamide (63 mg, 0.23 mmol), Pd₂(dba)₃ (21 mg, 0.023 mmol), BrettPhos (25 mg, 0.046 mmol), and cesium carbonate (152 mg, 0.47 mmol) were mixed in 1,4-dioxane (1.2 mL) and heated in a microwave reactor for 90 minutes at 120° C. The reaction mixture was concentrated and purified by MPLC to give compound 35, N-[(oxetan-3-yl)methyl]-3-[(6-phenylpyridazin-3-yl)amino]benzamide (27 mg, 28%) as a yellowish white solid.

Synthesis of Compound 36

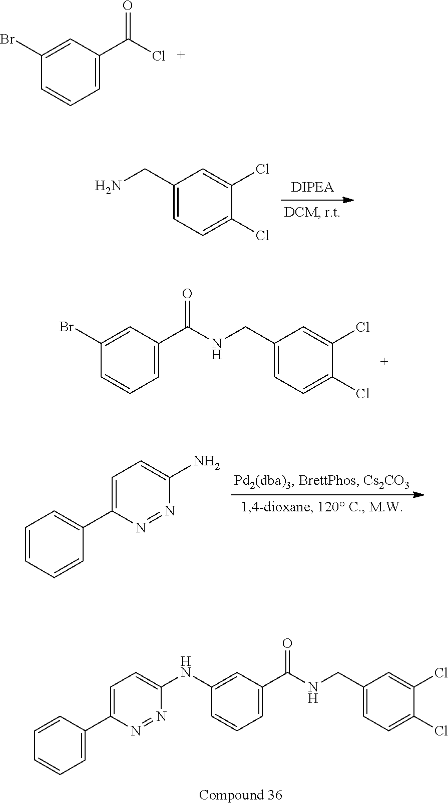

Step 1: 3-Bromobenzoyl chloride (0.12 mL, 0.91 mmol) and (3,4-dichlorophenyl)methanamine (134 mg, 0.76 mmol) were dissolved in DCM (7.6 mL), followed up by addition of DIPEA (0.28 mL, 1.63 mmol) and stirred for 22 hours at room temperature. The reaction mixture was concentrated and purified by MPLC to give 3-bromo-N-(3,4-dichlorobenzyl)benzamide (268 mg, 98%) as a white solid.

Step 2: 6-Phenylpyridazin-3-amine (40 mg, 0.23 mmol), 3-bromo-N-(3,4-dichlorobenzyl)benzamide (94 mg, 0.23 mmol), Pd₂(dba)₃ (21 mg, 0.023 mmol), BrettPhos (25 mg, 0.046 mmol), and cesium carbonate (152 mg, 0.47 mmol) were mixed in 1,4-dioxane (1.2 mL) and heated in a microwave reactor for 90 minutes at 120° C. The reaction mixture was concentrated and purified by MPLC. The crude mixture was solidified by using EA to give compound 36, N-(3,4-dichlorobenzyl)-3-((6-phenylpyridazin-3-yl)amino)benzamide (20 mg, 19%) as a beige solid.

Synthesis of Compound 37

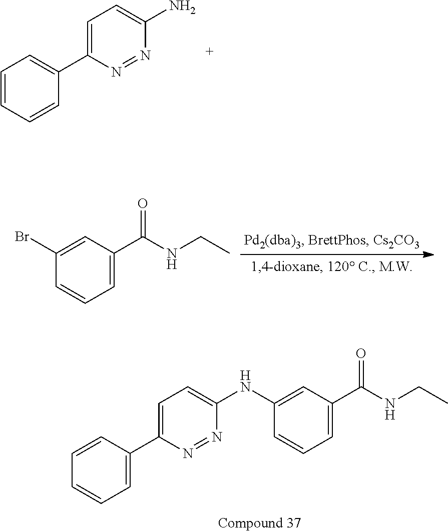 [SMILES: Nc1ccc(-c2ccccc2)nn1 ; CCNC(=O)c1cccc(Br)c1 ; CCNC(=O)c1cccc(Nc2ccc(-c3ccccc3)nn2)c1]

6-Phenylpyridazin-3-amine (40 mg, 0.23 mmol), 3-bromo-N-ethylbenzamide (64 mg, 0.28 mmol), Pd₂(dba)₃ (21 mg, 0.023 mmol), BrettPhos (25 mg, 0.046 mmol), and cesium carbonate (152 mg, 0.47 mmol) were mixed in 1,4-dioxane (1.2 mL) and heated in a microwave reactor for 90 minutes at 120° C. The reaction mixture was concentrated and purified by MPLC. The crude mixture was solidified by using EA and DCM to give compound 37, N-ethyl-3-[(6-phenylpyridazin-3-yl)amino]benzamide (14 mg, 19%) as a beige solid.

Synthesis of Compound 38

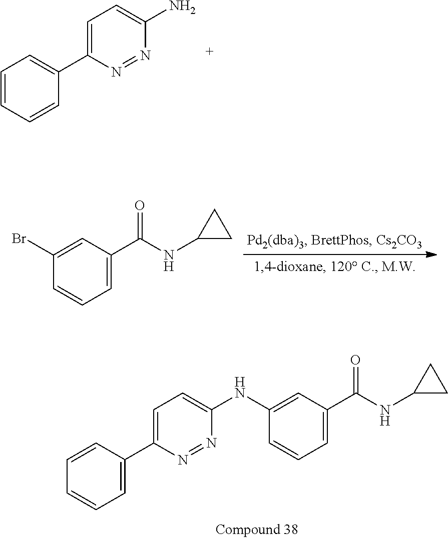 [SMILES: Nc1ccc(-c2ccccc2)nn1 ; O=C(NC1CC1)c1cccc(Br)c1 ; O=C(NC1CC1)c1cccc(Nc2ccc(-c3ccccc3)nn2)c1]

6-Phenylpyridazin-3-amine (40 mg, 0.23 mmol), 3-bromo-N-cyclopropylbenzamide (67 mg, 0.28 mmol), Pd₂(dba)₃ (21 mg, 0.023 mmol), BrettPhos (25 mg, 0.046 mmol), and cesium carbonate (152 mg, 0.47 mmol) were mixed in 1,4-dioxane (1.2 mL) and heated in a microwave reactor for 90 minutes at 120° C. The reaction mixture was concentrated and purified by MPLC. The crude mixture was solidified by using EA to give compound 38, N-cyclopropyl-3-[(6-phenylpyridazin-3-yl)amino]benzamide (25 mg, 33%) as a white solid.

Synthesis of Compound 39

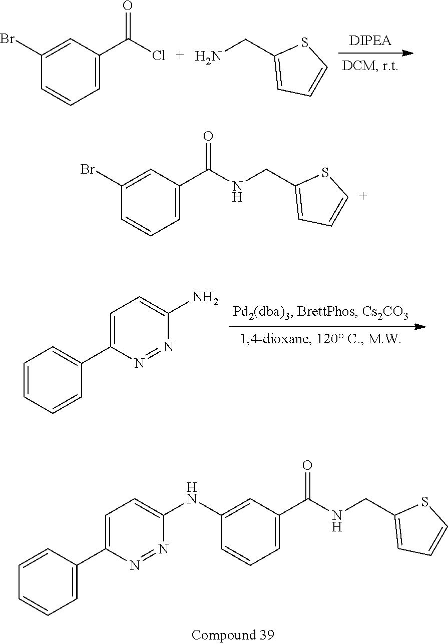

Step 1: 3-Bromobenzoyl chloride (0.12 mL, 0.91 mmol) and thiophen-2-ylmethanamine (86 mg, 0.76 mmol) were dissolved in DCM (7.6 mL), followed up by addition of DIPEA (0.28 mL, 1.63 mmol) and stirred for 18 hours at room temperature. The reaction mixture was concentrated and purified by MPLC to give 3-bromo-N-(thiophen-2-ylmethyl)benzamide (206 mg, 92%) as a beige solid.

Step 2: 6-Phenylpyridazin-3-amine (40 mg, 0.23 mmol), 3-bromo-N-(thiophen-2-ylmethyl)benzamide (76 mg, 0.26 mmol), Pd₂(dba)₃ (21 mg, 0.023 mmol), BrettPhos (25 mg, 0.046 mmol), and cesium carbonate (152 mg, 0.47 mmol) were mixed in 1,4-dioxane (1.2 mL) and heated in a microwave reactor for 90 minutes at 120° C. The reaction mixture was concentrated and purified by MPLC. The crude mixture was solidified by using EA to give compound 39, 3-[(6-phenylpyridazin-3-yl)amino]-N-[(thiophen-2-yl)methyl]benzamide (29 mg, 32%) as a white solid.

Synthesis of Compound 40

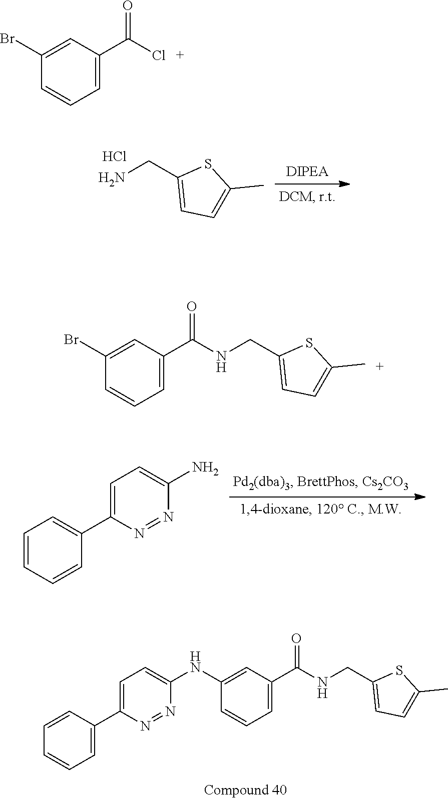

Step 1: 3-Bromobenzoyl chloride (0.12 mL, 0.91 mmol) and (5-methylthiophen-2-yl)methanamine hydrochloride (54 mg, 0.76 mmol) were dissolved in DCM (7.6 mL), followed up by addition of DIPEA (0.28 mL, 2.4 mmol) and stirred for 25 hours at room temperature. The reaction mixture was concentrated and purified by MPLC to give 3-bromo-N-((5-methylthiophen-2-yl)methyl)benzamide (236 mg, >99%) as a beige solid.

Step 2: 6-Phenylpyridazin-3-amine (40 mg, 0.23 mmol), 3-bromo-N-((5-methylthiophen-2-yl)methyl)benzamide (80 mg, 0.26 mmol), Pd₂(dba)₃ (21 mg, 0.023 mmol), BrettPhos (25 mg, 0.046 mmol), and cesium carbonate (152 mg, 0.47 mmol) were mixed in 1,4-dioxane (1.2 mL) and heated in a microwave reactor for 90 minutes at 120° C. The reaction mixture was concentrated and purified by MPLC. The crude mixture was solidified by using EA to give compound 40, N-[(5-methylthiophen-2-yl)methyl]-3-[(6-phenylpyridazin-3-yl)amino]benzamide (20 mg, 21%) as a beige solid.

Synthesis of Compound 41

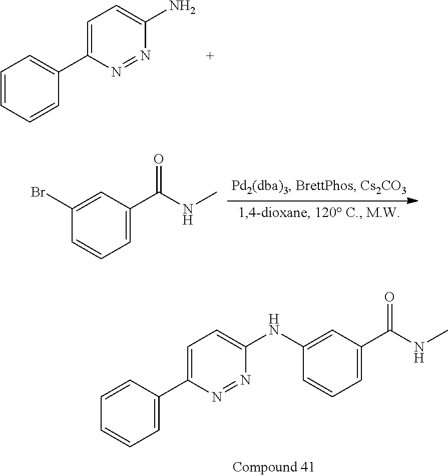 [SMILES: Nc1ccc(-c2ccccc2)nn1 ; "+" ; CNC(=O)c1cccc(Br)c1 ; CNC(=O)c1cccc(Nc2ccc(-c3ccccc3)nn2)c1]

6-Phenylpyridazin-3-amine (50 mg, 0.29 mmol), 3-bromo-N-methylbenzamide (188 mg, 0.88 mmol), Pd₂(dba)₃ (27 mg, 0.03 mmol), BrettPhos (31 mg, 0.06 mmol), and cesium carbonate (186 mg, 0.57 mmol) were mixed in 1,4-dioxane (1.5 mL) and heated in a microwave reactor for 90 minutes at 120° C. The reaction mixture was concentrated and purified by MPLC to give compound 41, N-methyl-3-[(6-phenylpyridazin-3-yl)amino]benzamide (10 mg, 11%) as a brown solid.

Synthesis of Compound 42

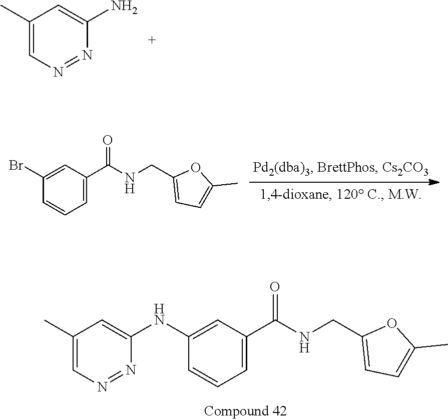

5-Methylpyridazin-3-amine (35 mg, 0.32 mmol), 3-bromo-N-((5-methylfuran-2-yl)methyl)benzamide (123 mg, 0.42 mmol), Pd₂(dba)₃ (29 mg, 0.03 mmol), BrettPhos (34 mg, 0.06 mmol), and cesium carbonate (209 mg, 0.64 mmol) were mixed in 1,4-dioxane (1.6 mL) and heated in a microwave reactor for 90 minutes at 120° C. The reaction mixture was concentrated and purified by MPLC. The crude mixture was solidified by using EA to give compound 42, N-[(5-methylfuran-2-yl)methyl]-3-[(5-methylpyridazin-3-yl)amino]benzamide (40 mg, 39%) as a beige solid.

Synthesis of Compound 43

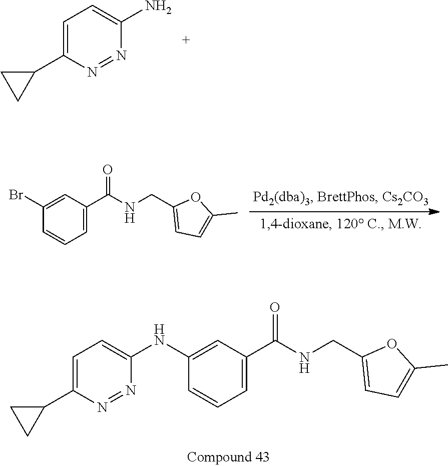 [SMILES: Nc1ccc(C2CC2)nn1 ; "+" ; Cc1ccc(CNC(=O)c2cccc(Br)c2)o1 ; Cc1ccc(CNC(=O)c2cccc(Nc3ccc(C4CC4)nn3)c2)o1]

6-Cyclopropylpyridazin-3-amine (40 mg, 0.3 mmol), 3-bromo-N-((5-methylfuran-2-yl)methyl)benzamide (111 mg, 0.38 mmol), Pd₂(dba)₃ (27 mg, 0.03 mmol), BrettPhos (32 mg, 0.06 mmol), and cesium carbonate (193 mg, 0.59 mmol) were mixed in 1,4-dioxane (1.5 mL) and heated in a microwave reactor for 90 minutes at 120° C. The reaction mixture was concentrated and purified by MPLC. The crude mixture was solidified by using EA to give compound 43, 3-[(6-cyclopropylpyridazin-3-yl)amino]-N-[(5-methylfuran-2-yl)methyl]benzamide (47 mg, 45%) as a beige solid.

Synthesis of Compound 44

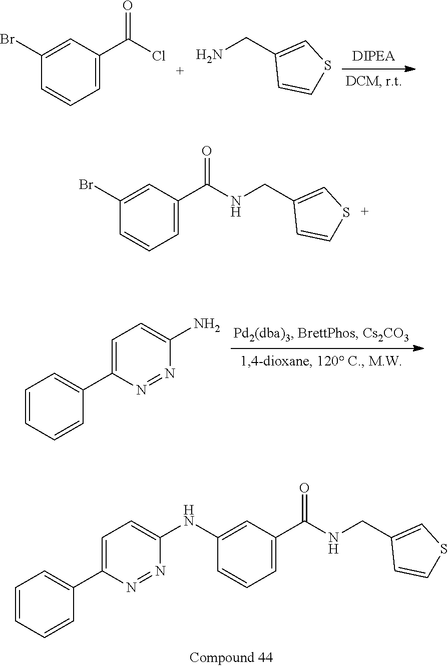

Step 1: 3-Bromobenzoyl chloride (0.12 mL, 0.91 mmol) and thiophen-3-ylmethanamine (0.075 mL, 0.76 mmol) were dissolved in DCM (7.6 mL), followed up by addition of DIPEA (0.28 mL, 1.63 mmol) and stirred for 23 hours at room temperature. The reaction mixture was extracted by EA and saturated aq. NH₄Cl. The organic layer was dried over anhydrous Na₂SO₄ and concentrated to give 3-bromo-N-(thiophen-3-ylmethyl)benzamide (259 mg, >99%) as a brown solid.

Step 2: 6-Phenylpyridazin-3-amine (40 mg, 0.23 mmol), 3-bromo-N-(thiophen-3-ylmethyl)benzamide (103 mg, 0.35 mmol), Pd₂(dba)₃ (21 mg, 0.023 mmol), BrettPhos (25 mg, 0.047 mmol), and cesium carbonate (152 mg, 0.47 mmol) were mixed in 1,4-dioxane (1.2 mL) and heated in a microwave reactor for 90 minutes at 120° C. The reaction mixture was concentrated and purified by MPLC to give compound 44, 3-[(6-phenylpyridazin-3-yl)amino]-N-[(thiophen-3-yl)methyl]benzamide (10 mg, 11%) as a beige solid.

Synthesis of Compound 45

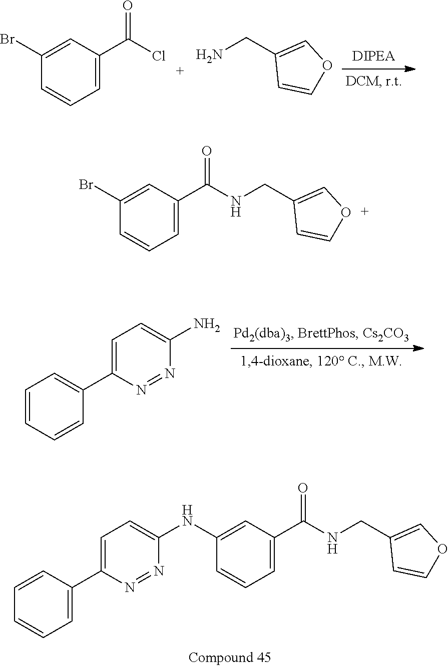

Step 1: 3-Bromobenzoyl chloride (0.12 mL, 0.91 mmol) and furan-3-ylmethanamine (0.082 mL, 0.76 mmol) were dissolved in DCM (7.6 mL), followed up by addition of DIPEA (0.28 mL, 1.63 mmol) and stirred for 28 hours at room temperature. The reaction mixture was extracted by EA and saturated aq. NH₄Cl. The organic layer was dried over anhydrous Na₂SO₄ and concentrated to give 3-bromo-N-(furan-3-ylmethyl)benzamide (252 mg, >99%) as a brown oil.

Step 2: 6-Phenylpyridazin-3-amine (40 mg, 0.23 mmol), 3-bromo-N-(furan-3-ylmethyl)benzamide (98 mg, 0.35 mmol), Pd₂(dba)₃ (21 mg, 0.023 mmol), BrettPhos (25 mg, 0.047 mmol), and cesium carbonate (152 mg, 0.47 mmol) were mixed in 1,4-dioxane (1.2 mL) and heated in a microwave reactor for 90 minutes at 120° C. The reaction mixture was concentrated and purified by MPLC. The crude mixture was solidified by using EA to give compound 45, N-[(furan-3-yl)methyl]-3-[(6-phenylpyridazin-3-yl)amino]benzamide (33 mg, 38%) as a beige solid.

Synthesis of Compound 46

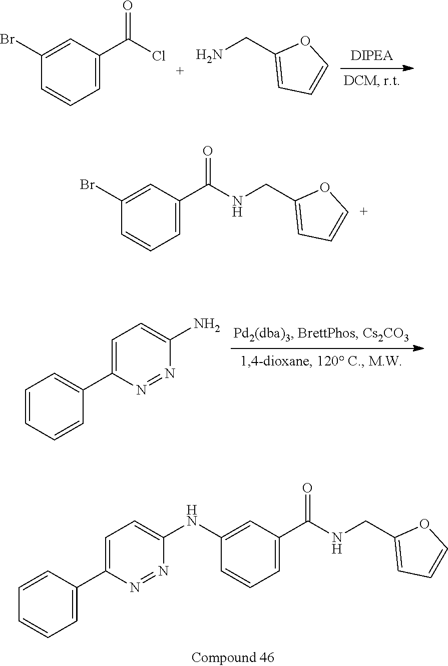

Step 1: 3-Bromobenzoyl chloride (0.12 mL, 0.91 mmol) and furan-2-ylmethanamine (0.08 mL, 0.76 mmol) were dissolved in DCM (7.6 mL), followed up by addition of DIPEA (0.28 mL, 1.63 mmol) and stirred for 25 hours at room temperature. The reaction mixture was extracted by EA and saturated aq. NH₄Cl. The organic layer was dried over anhydrous Na₂SO₄ and concentrated to give 3-bromo-N-(furan-2-ylmethyl)benzamide (285 mg, >99%) as a brown oil.

Step 2: 6-Phenylpyridazin-3-amine (40 mg, 0.23 mmol), 3-bromo-N-(furan-2-ylmethyl)benzamide (98 mg, 0.35 mmol), Pd₂(dba)₃ (21 mg, 0.023 mmol), BrettPhos (25 mg, 0.047 mmol), and cesium carbonate (152 mg, 0.47 mmol) were mixed in 1,4-dioxane (1.2 mL) and heated in a microwave reactor for 90 minutes at 120° C. The reaction mixture was concentrated and purified by MPLC. The crude mixture was solidified by using EA and HEX to give compound 46, N-[(furan-2-yl)methyl]-3-[(6-phenylpyridazin-3-yl)amino]benzamide (10 mg, 12%) as a beige solid.

Synthesis of Compound 47

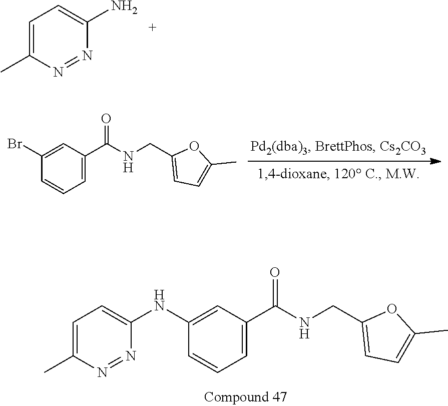

6-Methylpyridazin-3-amine (35 mg, 0.32 mmol), 3-bromo-N-((5-methylfuran-2-yl)methyl)benzamide (123 mg, 0.42 mmol), Pd₂(dba)₃ (29 mg, 0.03 mmol), BrettPhos (34 mg, 0.06 mmol), and cesium carbonate (209 mg, 0.64 mmol) were mixed in 1,4-dioxane (1.6 mL) and heated in a microwave reactor for 90 minutes at 120° C. The reaction mixture was concentrated and purified by MPLC. The crude mixture was solidified by using EA to give compound 47, N-[(5-methylfuran-2-yl)methyl]-3-[(6-methylpyridazin-3-yl)amino]benzamide (45 mg, 44%) as a beige solid.

Synthesis of Compound 48

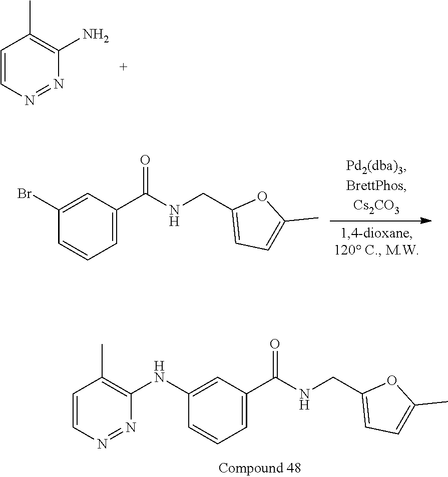

4-Methylpyridazin-3-amine (35 mg, 0.32 mmol), 3-bromo-N-((5-methylfuran-2-yl)methyl)benzamide (123 mg, 0.42 mmol), Pd₂(dba)₃ (29 mg, 0.03 mmol), BrettPhos (34 mg, 0.06 mmol), and cesium carbonate (209 mg, 0.64 mmol) were mixed in 1,4-dioxane (1.6 mL) and heated in a microwave reactor for 90 minutes at 120° C. The reaction mixture was concentrated and purified by MPLC. The crude mixture was solidified by using EA to give compound 48, N-[(5-methylfuran-2-yl)methyl]-3-[(4-methylpyridazin-3-yl)amino]benzamide (35 mg, 34%) as a beige solid.

Synthesis of Compound 49

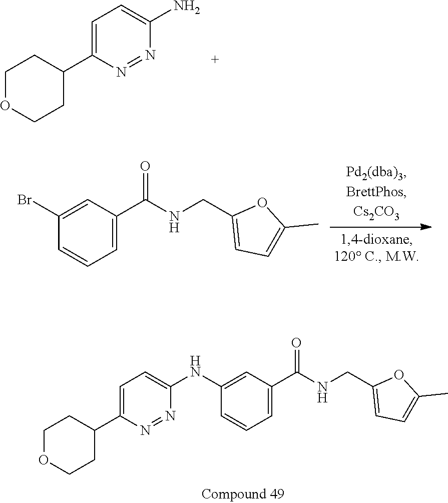 [SMILES: Nc1ccc(C2CCOCC2)nn1 ; "+" ; Cc1ccc(CNC(=O)c2cccc(Br)c2)o1 ; Cc1ccc(CNC(=O)c2cccc(Nc3ccc(C4CCOCC4)nn3)c2)o1]

6-(Tetrahydro-2H-pyran-4-yl)pyridazin-3-amine (40 mg, 0.22 mmol), 3-bromo-N-((5-methylfuran-2-yl)methyl)benzamide (85 mg, 0.29 mmol), Pd₂(dba)₃ (20 mg, 0.021 mmol), BrettPhos (23 mg, 0.042 mmol), and cesium carbonate (145 mg, 0.45 mmol) were mixed in 1,4-dioxane (1.1 mL) and heated in a microwave reactor for 90 minutes at 120° C. The reaction mixture was concentrated and purified by MPLC. The crude mixture was solidified by using EA to give compound 49, N-[(5-methylfuran-2-yl)methyl]-3-{[6-(oxan-4-yl)pyridazin-3-yl]amino}benzamide (42 mg, 48%) as a white solid.

Synthesis of Compound 50

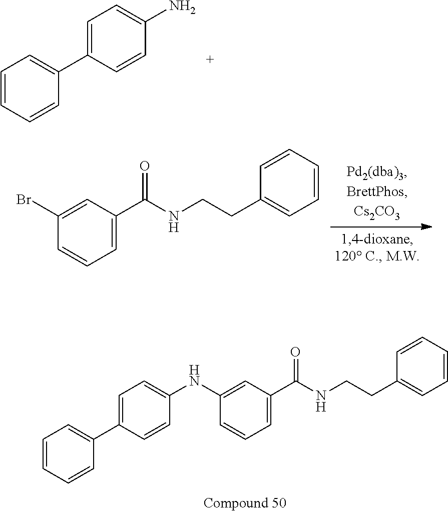 [SMILES: Nc1ccc(-c2ccccc2)cc1 ; "+" ; O=C(NCCc1ccccc1)c1cccc(Br)c1 ; O=C(NCCc1ccccc1)c1cccc(Nc2ccc(-c3ccccc3)cc2)c1]

[1,1′-Biphenyl]-4-amine (45 mg, 0.27 mmol), 3-bromo-N-phenethylbenzamide (97 mg, 0.32 mmol), Pd₂(dba)₃ (24 mg, 0.27 mmol), BrettPhos (29 mg, 0.053 mmol), and cesium carbonate (173 mg, 0.53 mmol) were mixed in 1,4-dioxane (1.3 mL) and heated in a microwave reactor for 90 minutes at 120° C. The reaction mixture was concentrated and purified by MPLC. The crude mixture was solidified by using EA to give compound 50, 3-({[1,1′-biphenyl]-4-yl}amino)-N-(2-phenylethyl)benzamide (29 mg, 28%) as a grey solid.

Synthesis of Compound 51

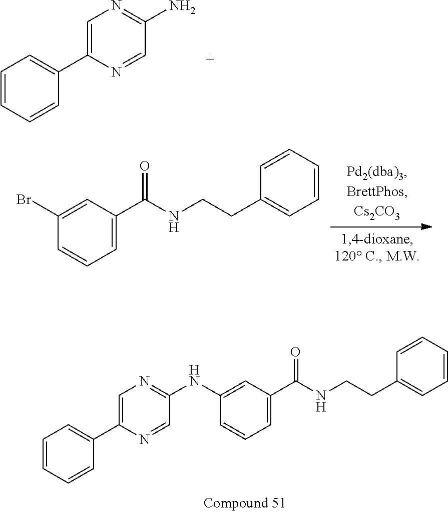 [SMILES: Nc1cnc(-c2ccccc2)cn1 ; "+" ; O=C(NCCc1ccccc1)c1cccc(Br)c1 ; O=C(NCCc1ccccc1)c1cccc(Nc2cnc(-c3ccccc3)cn2)c1]

5-Phenylpyrazin-2-amine (45 mg, 0.26 mmol), 3-bromo-N-phenethylbenzamide (96 mg, 0.32 mmol), Pd₂(dba)₃ (24 mg, 0.026 mmol), BrettPhos (28 mg, 0.053 mmol), and cesium carbonate (171 mg, 0.53 mmol) were mixed in 1,4-dioxane (1.3 mL) and heated in a microwave reactor for 90 minutes at 120° C. The reaction mixture was concentrated and purified by MPLC. The crude mixture was solidified by using EA to give compound 51, N-(2-phenylethyl)-3-[(5-phenylpyrazin-2-yl)amino]benzamide (20 mg, 19%) as a yellowish white solid.

Synthesis of Compound 52

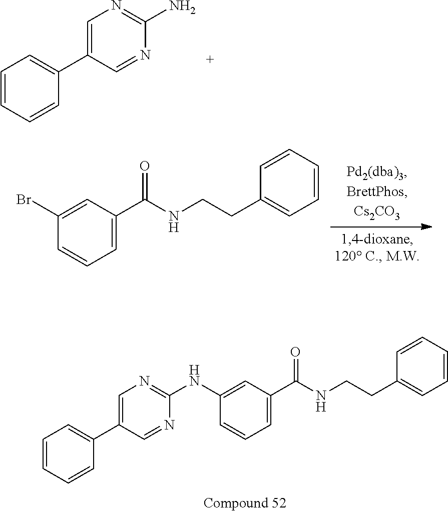 [SMILES: Nc1ncc(-c2ccccc2)cn1 ; "+" ; O=C(NCCc1ccccc1)c1cccc(Br)c1 ; O=C(NCCc1ccccc1)c1cccc(Nc2ncc(-c3ccccc3)cn2)c1]

5-Phenylpyrimidin-2-amine (45 mg, 0.26 mmol), 3-bromo-N-phenethylbenzamide (96 mg, 0.32 mmol), Pd₂(dba)₃ (24 mg, 0.026 mmol), BrettPhos (28 mg, 0.053 mmol), and cesium carbonate (171 mg, 0.53 mmol) were mixed in 1,4-dioxane (1.3 mL) and heated in a microwave reactor for 90 minutes at 120° C. The reaction mixture was concentrated and purified by MPLC. The crude mixture was solidified by using EA to give compound 52, N-(2-phenylethyl)-3-[(5-phenylpyrimidin-2-yl)amino]benzamide (59 mg, 57%) as a beige solid.

Synthesis of Compound 53

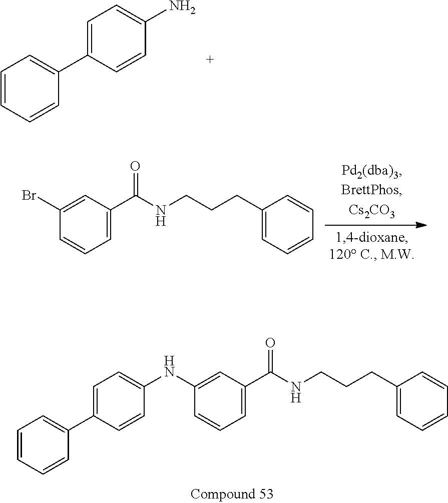 [SMILES: Nc1ccc(-c2ccccc2)cc1 ; "+" ; O=C(NCCCc1ccccc1)c1cccc(Br)c1 ; O=C(NCCCc1ccccc1)c1cccc(Nc2ccc(-c3ccccc3)cc2)c1]

[1,1′-Biphenyl]-4-amine (45 mg, 0.27 mmol), 3-bromo-N-(3-phenylpropyl)benzamide (121 mg, 0.4 mmol), Pd₂(dba)₃ (24 mg, 0.027 mmol), BrettPhos (28 mg, 0.053 mmol), and cesium carbonate (173 mg, 0.53 mmol) were mixed in 1,4-dioxane (1.3 mL) and heated in a microwave reactor for 90 minutes at 120° C. The reaction mixture was concentrated and purified by MPLC. The crude mixture was solidified by using EA and HEX to give compound 53, 3-({[1,1′-biphenyl]-4-yl}amino)-N-(3-phenylpropyl)benzamide (25 mg, 23%) as a white solid.

Synthesis of Compound 54

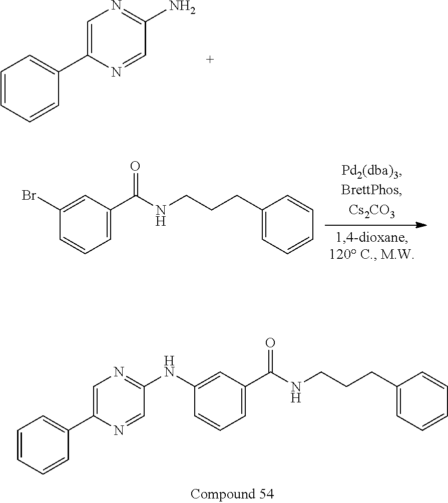 [SMILES: Nc1cnc(-c2ccccc2)cn1 ; "+" ; O=C(NCCCc1ccccc1)c1cccc(Br)c1 ; O=C(NCCCc1ccccc1)c1cccc(Nc2cnc(-c3ccccc3)cn2)c1]

5-Phenylpyrazin-2-amine (45 mg, 0.27 mmol), 3-bromo-N-(3-phenylpropyl)benzamide (121 mg, 0.4 mmol), Pd₂(dba)₃ (24 mg, 0.027 mmol), BrettPhos (28 mg, 0.053 mmol), and cesium carbonate (173 mg, 0.53 mmol) were mixed in 1,4-dioxane (1.3 mL) and heated in a microwave reactor for 90 minutes at 120° C. The reaction mixture was concentrated and purified by MPLC. The crude mixture was solidified by using EA to give compound 54, N-(3-phenylpropyl)-3-[(5-phenylpyrazin-2-yl)amino]benzamide (37 mg, 35%) as a yellowish white solid.

Synthesis of Compound 55

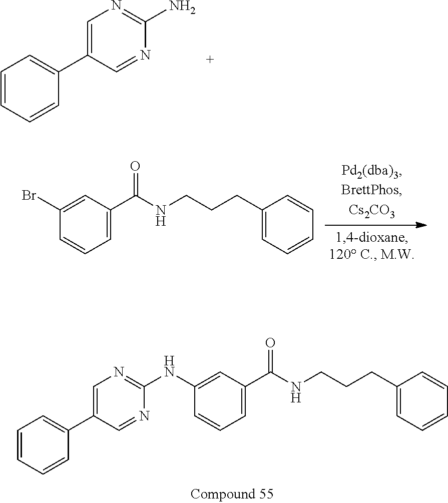 [SMILES: Nc1ncc(-c2ccccc2)cn1 ; "+" ; O=C(NCCCc1ccccc1)c1cccc(Br)c1 ; O=C(NCCCc1ccccc1)c1cccc(Nc2ncc(-c3ccccc3)cn2)c1]

5-Phenylpyrimidin-2-amine (45 mg, 0.26 mmol), 3-bromo-N-(3-phenylpropyl)benzamide (120 mg, 039 mmol), Pd₂(dba)₃ (24 mg, 0.026 mmol), BrettPhos (28 mg, 0.053 mmol), and cesium carbonate (171 mg, 0.53 mmol) were mixed in 1,4-dioxane (1.3 mL) and heated in a microwave reactor for 90 minutes at 120° C. The reaction mixture was concentrated and purified by MPLC. The crude mixture was solidified by using EA to give compound 55, N-(3-phenylpropyl)-3-[(5-phenylpyrimidin-2-yl)amino]benzamide (37 mg, 35%) as a beige solid.

Synthesis of Compound 57

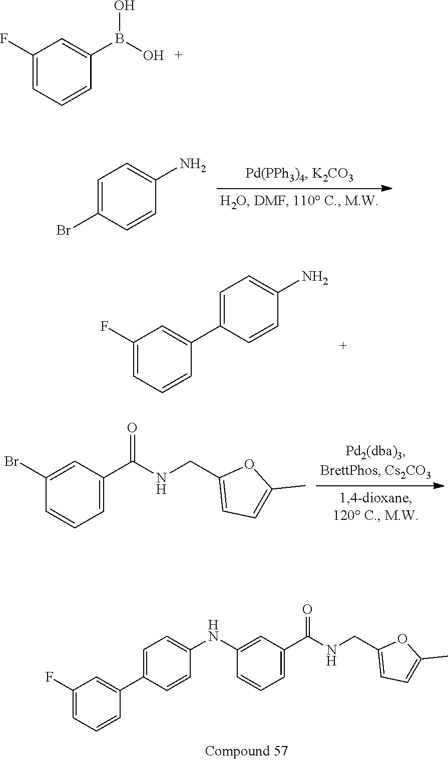

Step 1: (3-Fluorophenyl)boronic acid (300 mg, 2.1 mmol), 4-bromoaniline (307 mg, 1.79 mmol), Pd(PPh₃)₄ (103 mg, 0.09 mmol) and potassium carbonate (740 mg, 5.36 mmol) were mixed in H₂O/DMF (4.3/4.3 mL) and heated in a microwave reactor for 35 minutes at 110° C. The reaction mixture was concentrated and purified by MPLC to give 3′-fluoro-[1,1′-biphenyl]-4-amine (276 mg, 82%) as a beige solid.

Step 2: 3′-Fluoro-[1,1′-biphenyl]-4-amine (40 mg, 0.21 mmol), 3-bromo-N-((5-methylfuran-2-yl)methyl)benzamide (82 mg, 0.28 mmol), Pd₂(dba)₃ (20 mg, 0.02 mmol), BrettPhos (23 mg, 0.042 mmol), and cesium carbonate (139 mg, 0.43 mmol) were mixed in 1,4-dioxane (1.1 mL) and heated in a microwave reactor for 90 minutes at 120° C. The reaction mixture was concentrated and purified by MPLC. The crude mixture was solidified by using EA and HEX to give compound 57, 3-({3′-fluoro-[1,1′-biphenyl]-4-yl}amino)-N-[(5-methylfuran-2-yl)methyl]benzamide (30 mg, 35%) as a white solid.

Synthesis of Compound 58

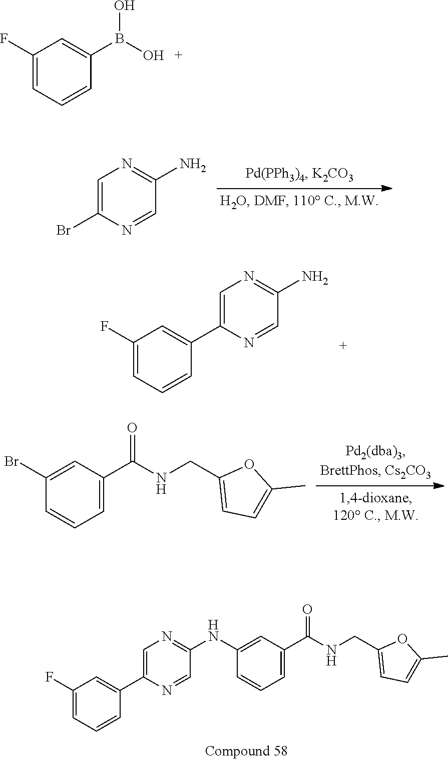

Step 1: (3-Fluorophenyl)boronic acid (300 mg, 2.1 mmol), 5-bromopyrazin-2-amine (311 mg, 1.79 mmol), Pd(PPh₃)₄ (103 mg, 0.09 mmol) and potassium carbonate (740 mg, 5.36 mmol) were mixed in H₂O/DMF (4.3/4.3 mL) and heated in a microwave reactor for 35 minutes at 110° C. The reaction mixture was concentrated and purified by MPLC to give 5-(3-fluorophenyl)pyrazin-2-amine (277 mg, 82%) as a yellowish white solid.

Step 2: 5-(3-Fluorophenyl)pyrazin-2-amine (40 mg, 0.23 mmol), 3-bromo-N-((5-methylfuran-2-yl)methyl)benzamide (80 mg, 0.27 mmol), Pd₂(dba)₃ (20 mg, 0.021 mmol), BrettPhos (23 mg, 0.042 mmol), and cesium carbonate (138 mg, 0.42 mmol) were mixed in 1,4-dioxane (1.1 mL) and heated in a microwave reactor for 90 minutes at 120° C. The reaction mixture was concentrated and purified by MPLC to give compound 58, 3-{[5-(3-fluorophenyl)pyrazin-2-yl]amino}-N-[(5-methylfuran-2-yl)methyl]benzamide (14 mg, 16%) as a brown solid.

Synthesis of Compound 60

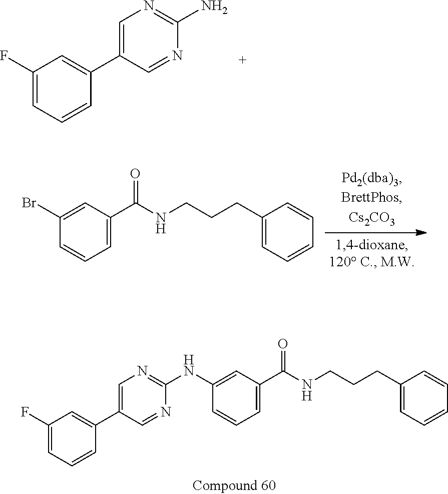 [SMILES: Nc1ncc(-c2cccc(F)c2)cn1 ; "+" ; O=C(NCCCc1ccccc1)c1cccc(Br)c1 ; O=C(NCCCc1ccccc1)c1cccc(Nc2ncc(-c3cccc(F)c3)cn2)c1]

5-(3-Fluorophenyl)pyrimidin-2-amine (45 mg, 0.24 mmol), 3-bromo-N-(3-phenylpropyl)benzamide (114 mg, 0.36 mmol), Pd₂(dba)₃ (22 mg, 0.024 mmol), BrettPhos (26 mg, 0.048 mmol), and cesium carbonate (155 mg, 0.48 mmol) were mixed in 1,4-dioxane (1.2 mL) and heated in a microwave reactor for 90 minutes at 120° C. The reaction mixture was concentrated and purified by MPLC. The crude mixture was solidified by using EA to give compound 60, 3-{[5-(3-fluorophenyl)pyrimidin-2-yl]amino}-N-(3-phenylpropyl)benzamide (30 mg, 30%) as a white solid.

Synthesis of Compound 61

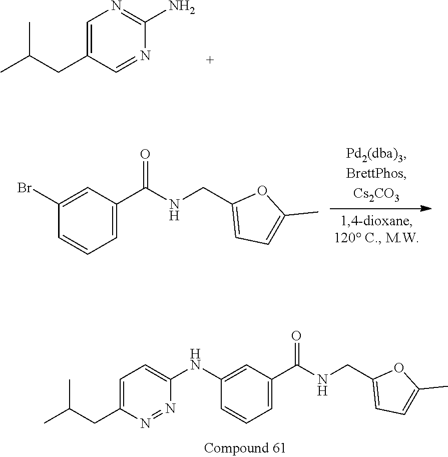

6-Isobutylpyridazin-3-amine (44 mg, 0.29 mmol), 3-bromo-N-((5-methylfuran-2-yl)methyl)benzamide (110 mg, 0.37 mmol), Pd₂(dba)₃ (26 mg, 0.03 mmol), BrettPhos (31 mg, 0.06 mmol), and cesium carbonate (188 mg, 0.58 mmol) were mixed in 1,4-dioxane (1.4 mL) and heated in a microwave reactor for 90 minutes at 120° C. The reaction mixture was concentrated and purified by MPLC. The crude mixture was solidified by using EA to give compound 61, N-[(5-methylfuran-2-yl)methyl]-3-{[6-(2-methylpropyl)pyridazin-3-yl]amino}benzamide (49 mg, 47%) as a beige solid.

Synthesis of Compound 62

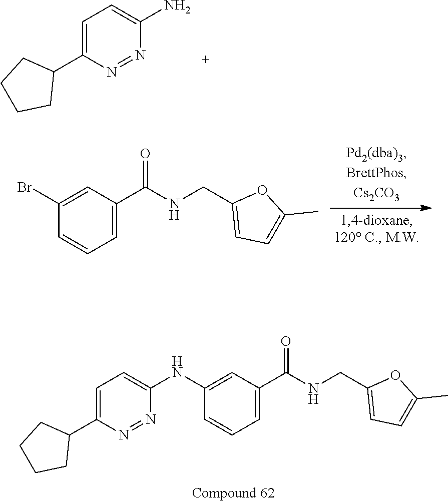 [SMILES: Nc1ccc(C2CCCC2)nn1 ; "+" ; Cc1ccc(CNC(=O)c2cccc(Br)c2)o1 ; Cc1ccc(CNC(=O)c2cccc(Nc3ccc(C4CCCC4)nn3)c2)o1]

6-Cyclopentylpyridazin-3-amine (47 mg, 0.29 mmol), 3-bromo-N-((5-methylfuran-2-yl)methyl)benzamide (110 mg, 0.37 mmol), Pd₂(dba)₃ (26 mg, 0.03 mmol), BrettPhos (31 mg, 0.06 mmol), and cesium carbonate (188 mg, 0.58 mmol) were mixed in 1,4-dioxane (1.4 mL) and heated in a microwave reactor for 90 minutes at 120° C. The reaction mixture was concentrated and purified by MPLC. The crude mixture was solidified by using EA to give compound 62, 3-[(6-cyclopentylpyridazin-3-yl)amino]-N-[(5-methylfuran-2-yl)methyl]benzamide (72 mg, 66%) as a beige solid.

Synthesis of Compound 63

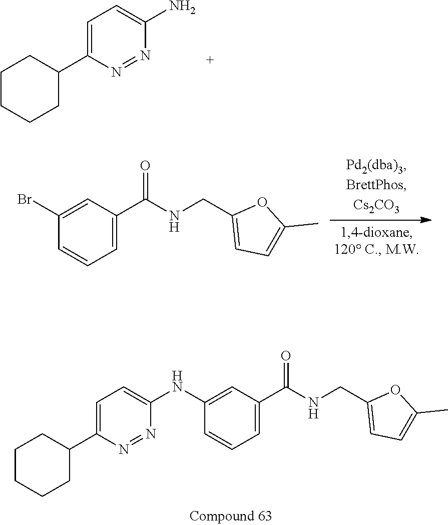 [SMILES: Nc1ccc(C2CCCCC2)nn1 ; "+" ; Cc1ccc(CNC(=O)c2cccc(Br)c2)o1 ; Cc1ccc(CNC(=O)c2cccc(Nc3ccc(C4CCCCC4)nn3)c2)o1]

6-Cyclohexylpyridazin-3-amine (51 mg, 0.29 mmol), 3-bromo-N-((5-methylfuran-2-yl)methyl)benzamide (110 mg, 0.37 mmol), Pd₂(dba)₃ (26 mg, 0.03 mmol), BrettPhos (31 mg, 0.06 mmol), and cesium carbonate (188 mg, 0.58 mmol) were mixed in 1,4-dioxane (1.4 mL) and heated in a microwave reactor for 90 minutes at 120° C. The reaction mixture was concentrated and purified by MPLC. The crude mixture was solidified by using EA to give compound 63, 3-[(6-cyclohexylpyridazin-3-yl)amino]-N-[(5-methylfuran-2-yl)methyl]benzamide (51 mg, 46%) as a beige solid.

Synthesis of Compound 65

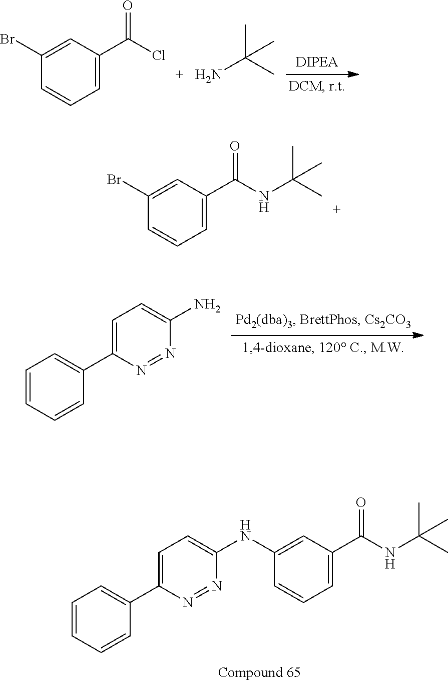

Step 1: 3-Bromobenzoyl chloride (0.12 mL, 0.91 mmol) and 2-methylpropan-2-amine (0.08 mL, 0.76 mmol) were dissolved in DCM (7.6 mL), followed up by addition of DIPEA (0.28 mL, 1.63 mmol) and stirred for 31 hours at room temperature. The reaction mixture was extracted by EA and saturated aq. NH₄Cl. The organic layer was dried over anhydrous Na₂SO₄ and concentrated to give 3-bromo-N-(tert-butyl)benzamide (209 mg, >99%) as a brown oil.

Step 2: 6-Phenylpyridazin-3-amine (45 mg, 0.26 mmol), 3-bromo-N-(tert-butyl)benzamide (88 mg, 0.34 mmol), Pd₂(dba)₃ (24 mg, 0.026 mmol), BrettPhos (28 mg, 0.053 mmol), and cesium carbonate (171 mg, 0.53 mmol) were mixed in 1,4-dioxane (1.3 mL) and heated in a microwave reactor for 90 minutes at 120° C. The reaction mixture was concentrated and purified by MPLC. The crude mixture was solidified by using EA and HEX to give compound 65, N-tert-butyl-3-[(6-phenylpyridazin-3-yl)amino]benzamide (14 mg, 15%) as a light orange solid.

Synthesis of Compound 66

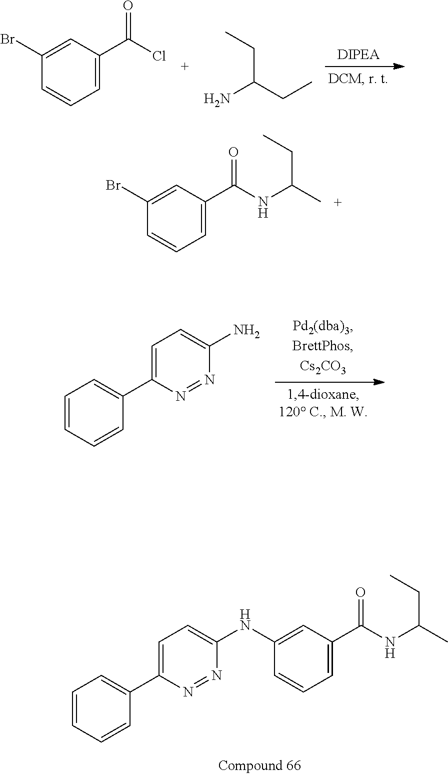

Step 1: 3-Bromobenzoyl chloride (0.12 mL, 0.91 mmol) and pentan-3-amine (0.09 mL, 0.76 mmol) were dissolved in DCM (7.6 mL), followed up by addition of DIPEA (0.28 mL, 1.63 mmol) and stirred for 31 hours at room temperature. The reaction mixture was extracted by EA and saturated aq. NH₄Cl. The organic layer was dried over anhydrous Na₂SO₄ and concentrated to give 3-bromo-N-(pentan-3-yl)benzamide (238 mg, >99%) as a brown oil.

Step 2: 6-Phenylpyridazin-3-amine (45 mg, 0.26 mmol), 3-bromo-N-(pentan-3-yl)benzamide (106 mg, 0.39 mmol), Pd₂(dba)₃ (24 mg, 0.026 mmol), BrettPhos (28 mg, 0.053 mmol), and cesium carbonate (171 mg, 0.53 mmol) were mixed in 1,4-dioxane (1.3 mL) and heated in a microwave reactor for 90 minutes at 120° C. The reaction mixture was concentrated and purified by MPLC. The crude mixture was solidified by using EA to give compound 66, N-(pentan-3-yl)-3-[(6-phenylpyridazin-3-yl)amino]benzamide (27 mg, 28%) as a beige solid.

Synthesis of Compound 69

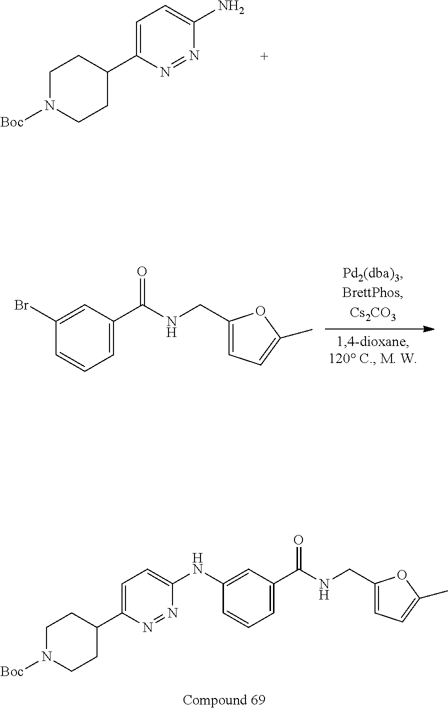 [SMILES: CC(C)(C)OC(=O)N1CCC(c2ccc(N)nn2)CC1 ; Cc1ccc(CNC(=O)c2cccc(Br)c2)o1 ; Cc1ccc(CNC(=O)c2cccc(Nc3ccc(C4CCN(C(=O)OC(C)(C)C)CC4)nn3)c2)o1]

tert-Butyl 4-(6-aminopyridazin-3-yl)piperidine-1-carboxylate (146 mg, 0.52 mmol), 3-bromo-N-((5-methylfuran-2-yl)methyl)benzamide (200 mg, 0.68 mmol), Pd₂(dba)₃ (48 mg, 0.05 mmol), BrettPhos (56 mg, 0.1 mmol), and cesium carbonate (341 mg, 1.05 mmol) were mixed in 1,4-dioxane (2.6 mL) and heated in a microwave reactor for 90 minutes at 120° C. The reaction mixture was concentrated and purified by MPLC. The crude mixture was solidified by using EA to give compound 69, tert-butyl 4-{6-[(3-{[(5-methylfuran-2-yl)methyl]carbamoyl}phenyl)amino]pyridazin-3-yl}piperidine-1-carboxylate (109 mg, 42%) as a beige solid.

Synthesis of Compound 70

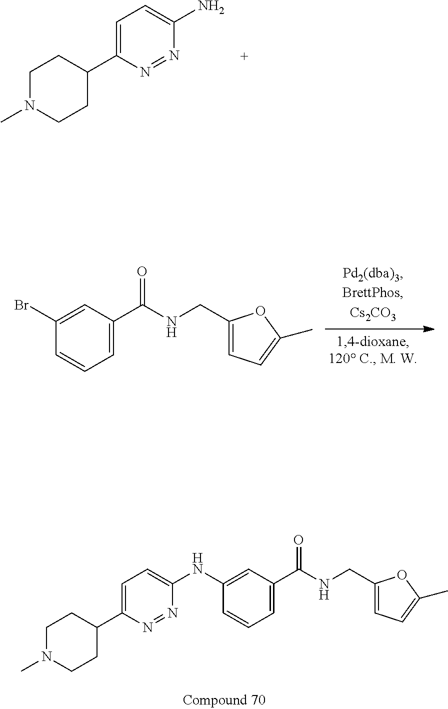

6-(1-Methylpiperidin-4-yl)pyridazin-3-amine (50 mg, 0.26 mmol), 3-bromo-N-((5-methylfuran-2-yl)methyl)benzamide (100 mg, 0.34 mmol), Pd₂(dba)₃ (24 mg, 0.03 mmol), BrettPhos (28 mg, 0.05 mmol), and cesium carbonate (170 mg, 0.52 mmol) were mixed in 1,4-dioxane (1.3 mL) and heated in a microwave reactor for 90 minutes at 120° C. The reaction mixture was concentrated and purified by MPLC. The crude mixture was solidified by using EA to give compound 70, N-[(5-methylfuran-2-yl)methyl]-3-{[6-(1-methylpiperidin-4-yl)pyridazin-3-yl]amino}benzamide (9 mg, 9%) as a beige solid.

Synthesis of Compound 71

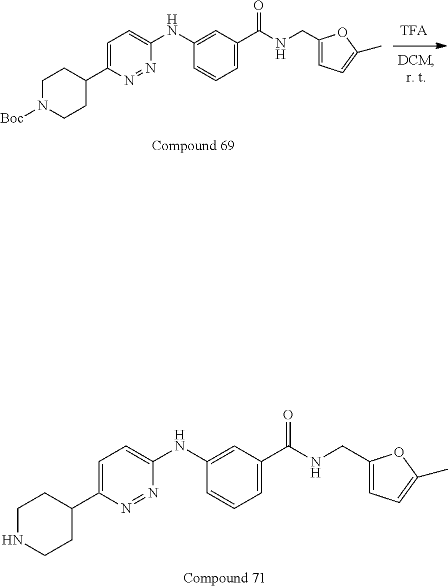 [SMILES: Cc1ccc(CNC(=O)c2cccc(Nc3ccc(C4CCN(C(=O)OC(C)(C)C)CC4)nn3)c2)o1 ; Cc1ccc(CNC(=O)c2cccc(Nc3ccc(C4CCNCC4)nn3)c2)o1]

tert-Butyl 4-(6-((3-(((5-methylfuran-2-yl)methyl)carbamoyl)phenyl)amino)pyridazin-3-yl)piperidine-1-carboxylate (30 mg, 0.61 mmol) was dissolved in DCM (3 mL), followed up by addition of trifluoroacetic acid (TFA) (0.5 mL, 0.12 M) and stirred for 1 hour at room temperature. The reaction mixture was extracted by DCM and saturated aq. NaHCO₃. The organic layer was dried over anhydrous Na₂SO₄ and concentrated to give compound 71, N-[(5-methylfuran-2-yl)methyl]-3-{[6-(piperidin-4-yl)pyridazin-3-yl]amino}benzamide (18 mg, 75%) as a beige foam.

Synthesis of Compound 72

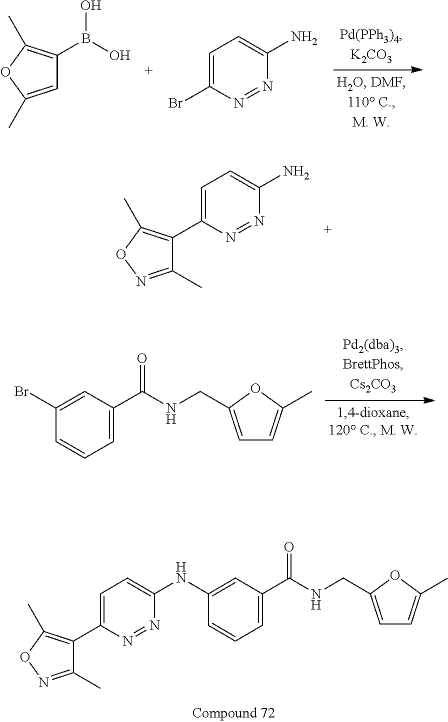

Step 1: (3,5-Dimethylisoxazol-4-yl)boronic acid (200 mg, 1.3 mmol), 6-bromopyridazin-3-amine (150 mg, 0.86 mmol), Pd(PPh₃)₄ (50 mg, 0.04 mmol) and potassium carbonate (357 mg, 2.59 mmol) were mixed in H₂O/DMF (1.7/1.7 mL) and heated in a microwave reactor for 35 minutes at 110° C. The reaction mixture was concentrated and purified by MPLC to give 6-(3,5-dimethylisoxazol-4-yl)pyridazin-3-amine (51 mg, 31%) as a white solid.

Step 2: 6-(3,5-Dimethylisoxazol-4-yl)pyridazin-3-amine (45 mg, 0.24 mmol), 3-bromo-N-((5-methylfuran-2-yl)methyl)benzamide (90 mg, 0.31 mmol), Pd₂(dba)₃ (22 mg, 0.024 mmol), BrettPhos (25 mg, 0.047 mmol), and cesium carbonate (153 mg, 0.47 mmol) were mixed in 1,4-dioxane (1.2 mL) and heated in a microwave reactor for 90 minutes at 120° C. The reaction mixture was concentrated and purified by MPLC. The crude mixture was solidified by using EA to give compound 72, 3-{[6-(3,5-dimethyl-1,2-oxazol-4-yl)pyridazin-3-yl]amino}-N-[(5-methylfuran-2-yl)methyl]benzamide (28 mg, 29%) as a beige solid.

Synthesis of Compound 73

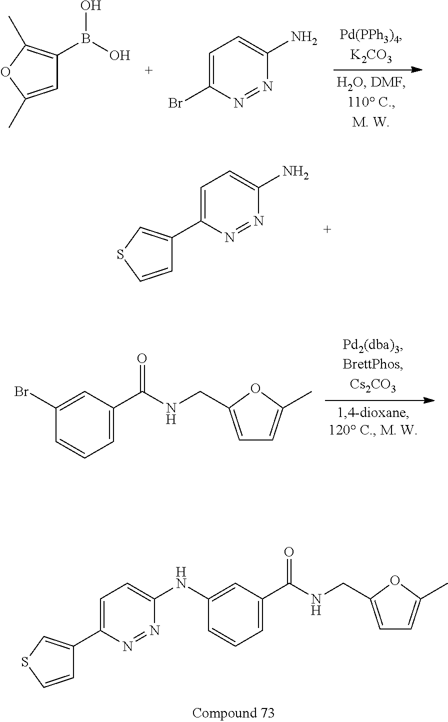

Step 1: Thiophen-3-ylboronic acid (132 mg, 1.03 mmol), 6-bromopyridazin-3-amine (150 mg, 0.86 mmol), Pd(PPh₃)₄ (50 mg, 0.04 mmol) and potassium carbonate (357 mg, 2.59 mmol) were mixed in H₂O/DMF (1.7/1.7 mL) and heated in a microwave reactor for 35 minutes at 110° C. The reaction mixture was concentrated and purified by MPLC to give 6-(thiophen-3-yl)pyridazin-3-amine (122 mg, 79%) as a yellowish white solid.

Step 2: 6-(Thiophen-3-yl)pyridazin-3-amine (42 mg, 0.24 mmol), 3-bromo-N-((5-methylfuran-2-yl)methyl)benzamide (90 mg, 0.31 mmol), Pd₂(dba)₃ (22 mg, 0.024 mmol), BrettPhos (25 mg, 0.047 mmol), and cesium carbonate (153 mg, 0.47 mmol) were mixed in 1,4-dioxane (1.2 mL) and heated in a microwave reactor for 90 minutes at 120° C. The reaction mixture was concentrated and purified by MPLC. The crude mixture was solidified by using EA to give compound 73, N-[(5-methylfuran-2-yl)methyl]-3-{[6-(thiophen-3-yl)pyridazin-3-yl]amino}benzamide (30 mg, 33%) as a beige solid.

Synthesis of Compound 74

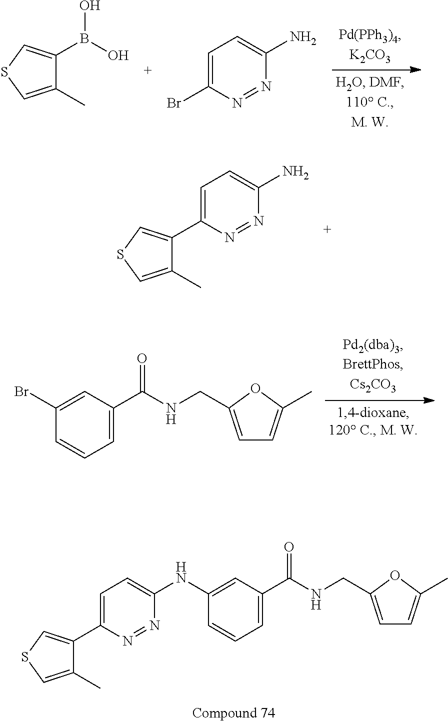

Step 1: (4-Methylthiophen-3-yl)boronic acid (147 mg, 1.03 mmol), 6-bromopyridazin-3-amine (150 mg, 0.86 mmol), Pd(PPh₃)₄ (50 mg, 0.04 mmol), and potassium carbonate (357 mg, 2.59 mmol) were mixed in H₂O/DMF (1.7/1.7 mL) and heated in a microwave reactor for 35 minutes at 110° C. The reaction mixture was concentrated and purified by MPLC to give 6-(4-methylthiophen-3-yl)pyridazin-3-amine (70 mg, 42%) as a beige solid.

Step 2: 6-(4-Methylthiophen-3-yl)pyridazin-3-amine (45 mg, 0.24 mmol), 3-bromo-N-((5-methylfuran-2-yl)methyl)benzamide (90 mg, 0.31 mmol), Pd₂(dba)₃ (22 mg, 0.024 mmol), BrettPhos (25 mg, 0.047 mmol), and cesium carbonate (153 mg, 0.47 mmol) were mixed in 1,4-dioxane (1.2 mL) and heated in a microwave reactor for 90 minutes at 120° C. The reaction mixture was concentrated and purified by MPLC. The crude mixture was solidified by using EA to give compound 74, N-[(5-methylfuran-2-yl)methyl]-3-{[6-(4-methylthiophen-3-yl)pyridazin-3-yl]amino}benzamide (23 mg, 24%) as a beige solid.

Synthesis of Compound 75

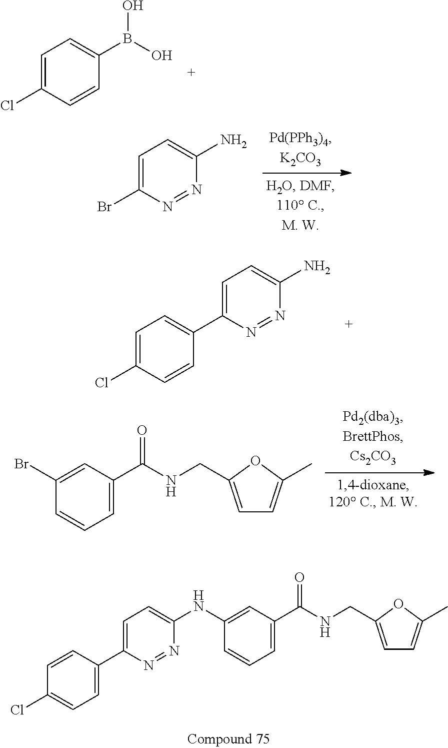 [SMILES: OB(O)c1ccc(Cl)cc1 ; Nc1ccc(Br)nn1 ; Nc1ccc(-c2ccc(Cl)cc2)nn1 ; Cc1ccc(CNC(=O)c2cccc(Br)c2)o1 ; Cc1ccc(CNC(=O)c2cccc(Nc3ccc(-c4ccc(Cl)cc4)nn3)c2)o1]

Step 1: (4-Chlorophenyl)boronic acid (200 mg, 1.28 mmol), 6-bromopyridazin-3-amine (290 mg, 1.66 mmol), Pd(PPh₃)₄ (74 mg, 0.064 mmol), and potassium carbonate (530 mg, 3.84 mmol) were mixed in H₂O/DMF (2.6/2.6 mL) and heated in a microwave reactor for 35 minutes at 110° C. The reaction mixture was extracted by EA and brine. The organic layer was dried over anhydrous Na₂SO₄ and concentrated. The crude mixture was solidified by using EA and HEX to give 6-(4-chlorophenyl)pyridazin-3-amine (175 mg, 66%) as a yellow solid.

Step 2: 6-(4-Chlorophenyl)pyridazin-3-amine (48 mg, 0.24 mmol), 3-bromo-N-((5-methylfuran-2-yl)methyl)benzamide (90 mg, 0.31 mmol), Pd₂(dba)₃ (22 mg, 0.024 mmol), BrettPhos (25 mg, 0.047 mmol), and cesium carbonate (153 mg, 0.47 mmol) were mixed in 1,4-dioxane (1.2 mL) and heated in a microwave reactor for 90 minutes at 120° C. The reaction mixture was concentrated and purified by MPLC. The crude mixture was solidified by using EA to give compound 75, 3-{[6-(4-chlorophenyl)pyridazin-3-yl]amino}-N-[(5-methylfuran-2-yl)methyl]benzamide (29 mg, 29%) as a beige solid.

Synthesis of Compound 76

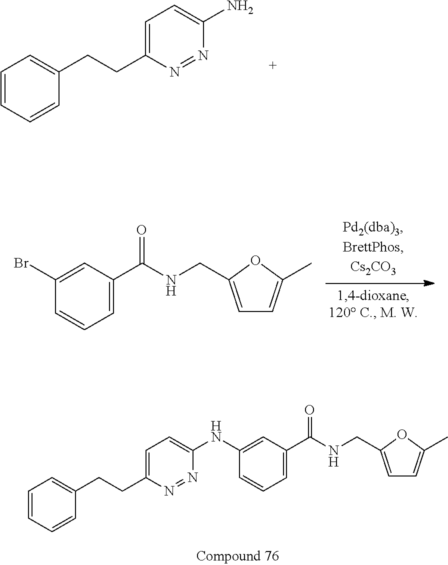 [SMILES: Nc1ccc(CCc2ccccc2)nn1 ; Cc1ccc(CNC(=O)c2cccc(Br)c2)o1 ; Cc1ccc(CNC(=O)c2cccc(Nc3ccc(CCc4ccccc4)nn3)c2)o1]

6-Phenethylpyridazin-3-amine (47 mg, 0.24 mmol), 3-bromo-N-((5-methylfuran-2-yl)methyl)benzamide (90 mg, 0.31 mmol), Pd₂(dba)₃ (22 mg, 0.024 mmol), BrettPhos (25 mg, 0.047 mmol), and cesium carbonate (153 mg, 0.47 mmol) were mixed in 1,4-dioxane (1.2 mL) and heated in a microwave reactor for 90 minutes at 120° C. The reaction mixture was concentrated and purified by MPLC. The crude mixture was solidified by using EA to give compound 76, N-[(5-methylfuran-2-yl)methyl]-3-{[6-(2-phenylethyl)pyridazin-3-yl]amino}benzamide (37 mg, 38%) as a white solid.

Synthesis of Compound 77

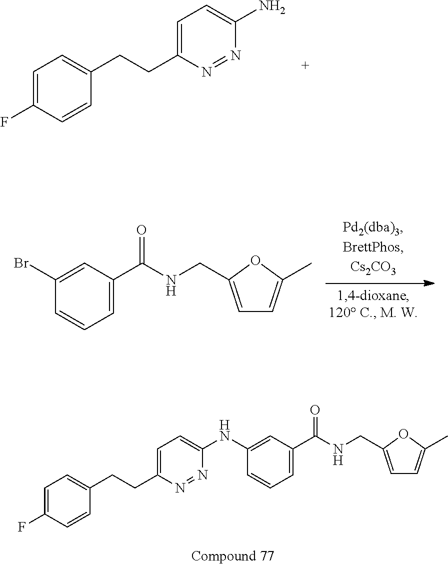 [SMILES: Nc1ccc(CCc2ccc(F)cc2)nn1 ; Cc1ccc(CNC(=O)c2cccc(Br)c2)o1 ; Cc1ccc(CNC(=O)c2cccc(Nc3ccc(CCc4ccc(F)cc4)nn3)c2)o1]

6-(4-Fluorophenethyl)pyridazin-3-amine (51 mg, 0.24 mmol), 3-bromo-N-((5-methylfuran-2-yl)methyl)benzamide (90 mg, 0.31 mmol), Pd₂(dba)₃ (22 mg, 0.024 mmol), BrettPhos (25 mg, 0.047 mmol), and cesium carbonate (153 mg, 0.47 mmol) were mixed in 1,4-dioxane (1.2 mL) and heated in a microwave reactor for 90 minutes at 120° C. The reaction mixture was concentrated and purified by MPLC. The crude mixture was solidified by using EA to give compound 77, 3-({6-[2-(4-fluorophenyl)ethyl]pyridazin-3-yl}amino)-N-[(5-methylfuran-2-yl)methyl]benzamide (33 mg, 33%) as a white solid.

Synthesis of Compound 78

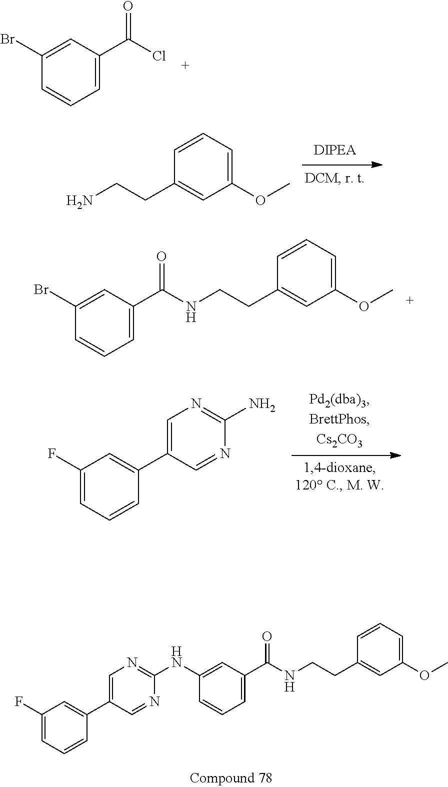 [SMILES: O=C(Cl)c1cccc(Br)c1 ; COc1cccc(CCN)c1 ; COc1cccc(CCNC(=O)c2cccc(Br)c2)c1 ; Nc1ncc(-c2cccc(F)c2)cn1 ; COc1cccc(CCNC(=O)c2cccc(Nc3ncc(-c4cccc(F)c4)cn3)c2)c1]

Step 1: 3-Bromobenzoyl chloride (0.12 mL, 0.91 mmol) and 2-(3-methoxyphenyl)ethan-1-amine (0.13 mL, 0.91 mmol) were dissolved in DCM (9.1 mL), followed up by addition of DIPEA (0.34 mL, 1.96 mmol) and stirred for 18 hours at room temperature. The reaction mixture was extracted by EA and saturated aq. NH₄Cl. The organic layer was dried over anhydrous Na₂SO₄ and concentrated to give 3-bromo-N-(3-methoxyphenethyl)benzamide (370 mg, >99%) as a yellow oil.

Step 2: 5-(3-Fluorophenyl)pyrimidin-2-amine (40 mg, 0.21 mmol), 3-bromo-N-(3-methoxyphenethyl)benzamide (103 mg, 0.25 mmol), Pd₂(dba)₃ (20 mg, 0.021 mmol), BrettPhos (23 mg, 0.042 mmol), and cesium carbonate (138 mg, 0.42 mmol) were mixed in 1,4-dioxane (1.1 mL) and heated in a microwave reactor for 90 minutes at 120° C. The reaction mixture was concentrated and purified by MPLC. The crude mixture was solidified by using EA to give compound 78, 3-{[5-(3-fluorophenyl)pyrimidin-2-yl]amino}-N-[2-(3-methoxyphenyl)ethyl]benzamide (13 mg, 14%) as a white solid.

Synthesis of Compound 80

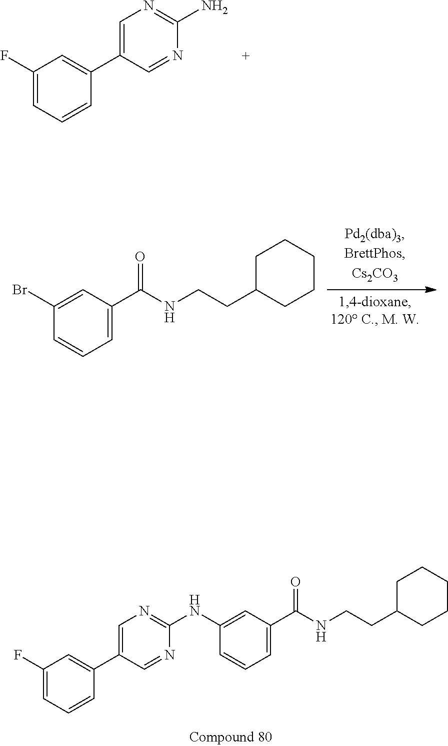 [SMILES: Nc1ncc(-c2cccc(F)c2)cn1 ; O=C(NCCC1CCCCC1)c1cccc(Br)c1 ; O=C(NCCC1CCCCC1)c1cccc(Nc2ncc(-c3cccc(F)c3)cn2)c1]

5-(3-Fluorophenyl)pyrimidin-2-amine (45 mg, 0.24 mmol), 3-bromo-N-(2-cyclohexylethyl)benzamide (94 mg, 0.29 mmol), Pd₂(dba)₃ (22 mg, 0.024 mmol), BrettPhos (26 mg, 0.048 mmol), and cesium carbonate (155 mg, 0.48 mmol) were mixed in 1,4-dioxane (1.2 mL) and heated in a microwave reactor for 90 minutes at 120° C. The reaction mixture was concentrated and purified by MPLC. The crude mixture was solidified by using EA to give compound 80, N-(2-cyclohexylethyl)-3-{[5-(3-fluorophenyl)pyrimidin-2-yl]amino}benzamide (32 mg, 32%) as a white solid.

Synthesis of Compound 82

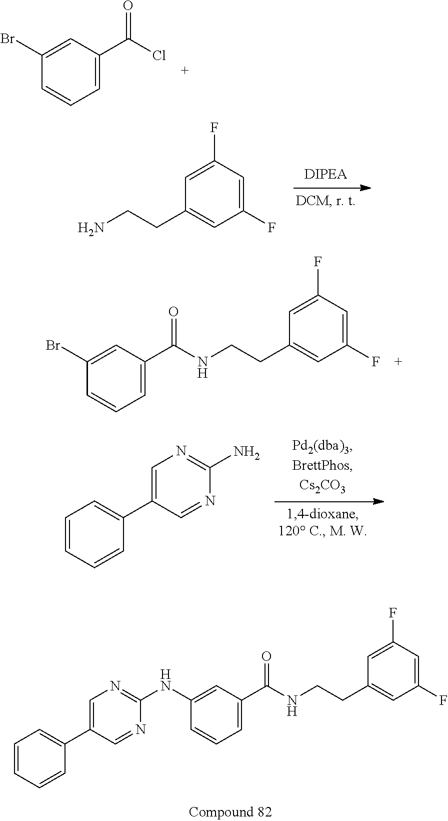 [SMILES: O=C(Cl)c1cccc(Br)c1 ; NCCc1cc(F)cc(F)c1 ; O=C(NCCc1cc(F)cc(F)c1)c1cccc(Br)c1 ; Nc1ncc(-c2ccccc2)cn1 ; O=C(NCCc1cc(F)cc(F)c1)c1cccc(Nc2ncc(-c3ccccc3)cn2)c1]

Step 1: 3-Bromobenzoyl chloride (0.12 mL, 0.91 mmol) and 2-(3,5-difluorophenyl)ethan-1-amine (0.12 mL, 0.91 mmol) were dissolved in DCM (9.1 mL), followed up by addition of DIPEA (0.34 mL, 1.96 mmol) and stirred for 22 hours at room temperature. The reaction mixture was extracted by EA and saturated aq. NH₄Cl. The organic layer was dried over anhydrous Na₂SO₄ and concentrated to give 3-bromo-N-(3,5-difluorophenethyl)benzamide (320 mg, >99%) as an orange solid.

Step 2: 5-Phenylpyrimidin-2-amine (40 mg, 0.23 mmol), 3-bromo-N-(3,5-difluorophenethyl)benzamide (99 mg, 0.28 mmol), Pd₂(dba)₃ (21 mg, 0.023 mmol), BrettPhos (25 mg, 0.047 mmol), and cesium carbonate (152 mg, 0.47 mmol) were mixed in 1,4-dioxane (1.2 mL) and heated in a microwave reactor for 90 minutes at 120° C. The reaction mixture was concentrated and purified by MPLC. The crude mixture was solidified by using EA to give compound 82, N-[2-(3,5-difluorophenyl)ethyl]-3-[(5-phenylpyrimidin-2-yl)amino]benzamide (26 mg, 26%) as a white solid.

Synthesis of Compound 83

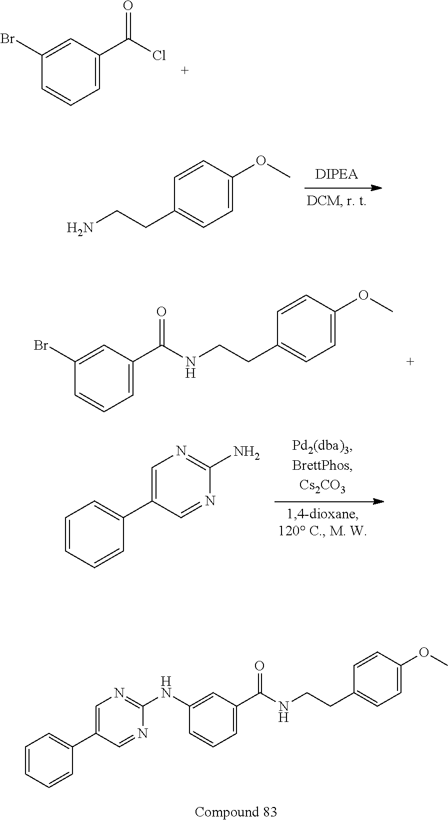 [SMILES: O=C(Cl)c1cccc(Br)c1 ; COc1ccc(CCN)cc1 ; COc1ccc(CCNC(=O)c2cccc(Br)c2)cc1 ; Nc1ncc(-c2ccccc2)cn1 ; COc1ccc(CCNC(=O)c2cccc(Nc3ncc(-c4ccccc4)cn3)c2)cc1]

Step 1: 3-Bromobenzoyl chloride (0.12 mL, 0.91 mmol) and 2-(4-methoxyphenyl)ethan-1-amine (0.13 mL, 0.91 mmol) were dissolved in DCM (9.1 mL), followed up by addition of DIPEA (0.34 mL, 1.96 mmol) and stirred for 22 hours at room temperature. The reaction mixture was extracted by EA and saturated aq. NH₄Cl. The organic layer was dried over anhydrous Na₂SO₄ and concentrated to give 3-bromo-N-(4-methoxyphenethyl)benzamide (325 mg, >99%) as a beige solid.

Step 2: 5-Phenylpyrimidin-2-amine (40 mg, 0.23 mmol), 3-bromo-N-(4-methoxyphenethyl)benzamide (100 mg, 0.28 mmol), Pd₂(dba)₃ (21 mg, 0.023 mmol), BrettPhos (25 mg, 0.047 mmol), and cesium carbonate (152 mg, 0.47 mmol) were mixed in 1,4-dioxane (1.2 mL) and heated in a microwave reactor for 90 minutes at 120° C. The reaction mixture was concentrated and purified by MPLC. The crude mixture was solidified by using EA and HEX to give compound 83, N-[2-(4-methoxyphenyl)ethyl]-3-[(5-phenylpyrimidin-2-yl)amino]benzamide (28 mg, 28%) as a white solid.

Synthesis of Compound 84

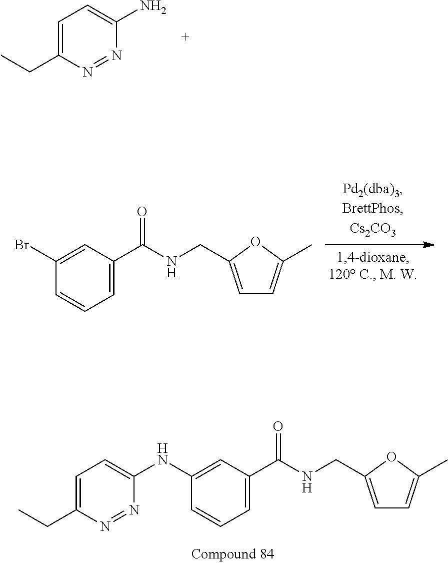

6-Ethylpyridazin-3-amine (28 mg, 0.23 mmol), 3-bromo-N-((5-methylfuran-2-yl)methyl)benzamide (80 mg, 0.27 mmol), Pd₂(dba)₃ (21 mg, 0.023 mmol), BrettPhos (24 mg, 0.045 mmol), and cesium carbonate (147 mg, 0.45 mmol) were mixed in 1,4-dioxane (1.1 mL) and heated in a microwave reactor for 90 minutes at 120° C. The reaction mixture was concentrated and purified by MPLC. The crude mixture was solidified by using EA and HEX to give compound 84, 3-[(6-ethylpyridazin-3-yl)amino]-N-[(5-methylfuran-2-yl)methyl]benzamide (42 mg, 48%) as a beige solid.

Synthesis of Compound 85

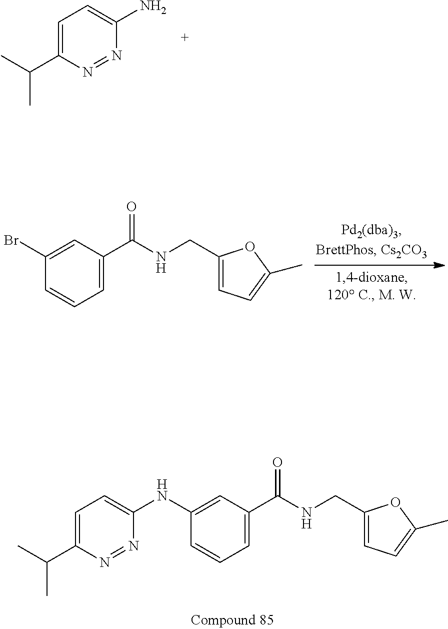

6-Isopropylpyridazin-3-amine (31 mg, 0.23 mmol), 3-bromo-N-((5-methylfuran-2-yl)methyl)benzamide (80 mg, 0.27 mmol), Pd₂(dba)₃ (21 mg, 0.023 mmol), BrettPhos (24 mg, 0.045 mmol), and cesium carbonate (147 mg, 0.45 mmol) were mixed in 1,4-dioxane (1.1 mL) and heated in a microwave reactor for 90 minutes at 120° C. The reaction mixture was concentrated and purified by MPLC. The crude mixture was solidified by using EA and HEX to give compound 85, N-[(5-methylfuran-2-yl)methyl]-3-{[6-(propan-2-yl)pyridazin-3-yl]amino}benzamide (21 mg, 26%) as a beige solid.

Synthesis of Compound 86

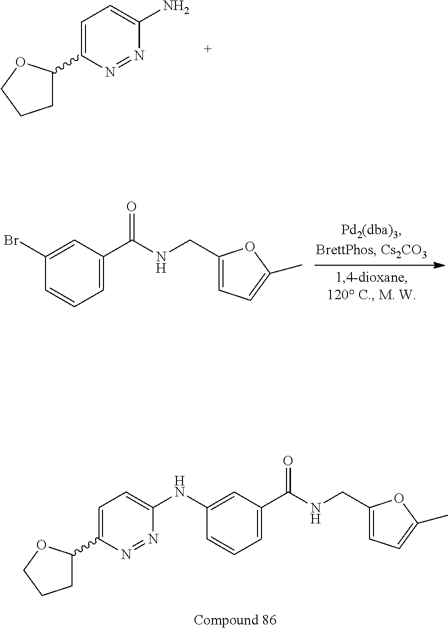 [SMILES: Nc1ccc(C2CCCO2)nn1 ; Cc1ccc(CNC(=O)c2cccc(Br)c2)o1 ; Cc1ccc(CNC(=O)c2cccc(Nc3ccc(C4CCCO4)nn3)c2)o1]

6-(Tetrahydrofuran-2-yl)pyridazin-3-amine (40 mg, 0.24 mmol), 3-bromo-N-((5-methylfuran-2-yl)methyl)benzamide (98 mg, 0.29 mmol), Pd₂(dba)₃ (30 mg, 0.024 mmol), BrettPhos (26 mg, 0.048 mmol), and cesium carbonate (158 mg, 0.48 mmol) were mixed in 1,4-dioxane (1.2 mL) and heated in a microwave reactor for 90 minutes at 120° C. The reaction mixture was concentrated and purified by MPLC. The crude mixture was solidified by using EA and HEX to give compound 86, N-[(5-methylfuran-2-yl)methyl]-3-{[6-(oxolan-2-yl)pyridazin-3-yl]amino}benzamide (20 mg, 22%) as a beige solid.

Synthesis of Compound 87

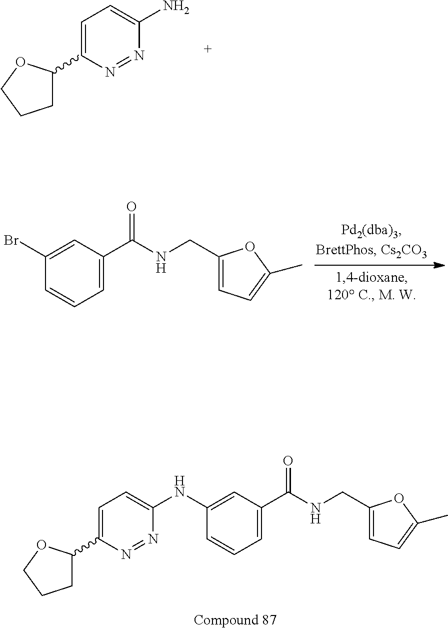 [SMILES: Nc1ccc(C2CCCO2)nn1 ; Cc1ccc(CNC(=O)c2cccc(Br)c2)o1 ; Cc1ccc(CNC(=O)c2cccc(Nc3ccc(C4CCCO4)nn3)c2)o1]

6-(Tetrahydrofuran-3-yl)pyridazin-3-amine (40 mg, 0.24 mmol), 3-bromo-N-((5-methylfuran-2-yl)methyl)benzamide (98 mg, 0.29 mmol), Pd₂(dba)₃ (30 mg, 0.024 mmol), BrettPhos (26 mg, 0.048 mmol), and cesium carbonate (158 mg, 0.48 mmol) were mixed in 1,4-dioxane (1.2 mL) and heated in a microwave reactor for 90 minutes at 120° C. The reaction mixture was concentrated and purified by MPLC. The crude mixture was solidified by using EA and HEX to give compound 87, N-[(5-methylfuran-2-yl)methyl]-3-{[6-(oxolan-3-yl)pyridazin-3-yl]amino}benzamide (44 mg, 48%) as a white solid.

Synthesis of Compound 88

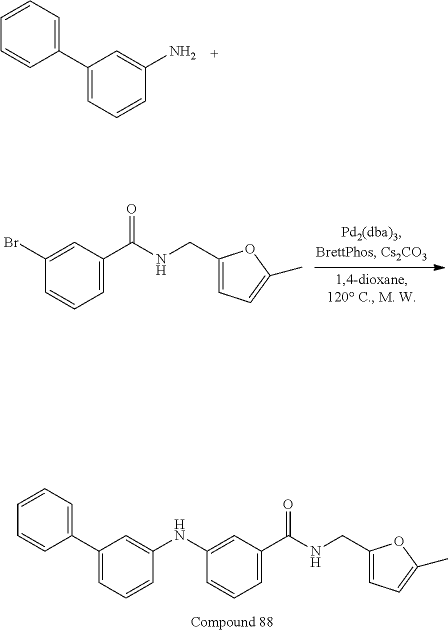 [SMILES: Nc1cccc(-c2ccccc2)c1 ; Cc1ccc(CNC(=O)c2cccc(Br)c2)o1 ; Cc1ccc(CNC(=O)c2cccc(Nc3cccc(-c4ccccc4)c3)c2)o1]

[1,1′-Biphenyl]-3-amine (40 mg, 0.24 mmol), 3-bromo-N-((5-methylfuran-2-yl)methyl)benzamide (96 mg, 0.28 mmol), Pd₂(dba)₃ (30 mg, 0.024 mmol), BrettPhos (25 mg, 0.047 mmol), and cesium carbonate (154 mg, 0.47 mmol) were mixed in 1,4-dioxane (1.2 mL) and heated in a microwave reactor for 90 minutes at 120° C. The reaction mixture was concentrated and purified by MPLC. The crude mixture was solidified by using EA and HEX to give compound 88, 3-({[1,1′-biphenyl]-3-yl}amino)-N-[(5-methylfuran-2-yl)methyl]benzamide (38 mg, 42%) as a beige solid.

Synthesis of Compound 89

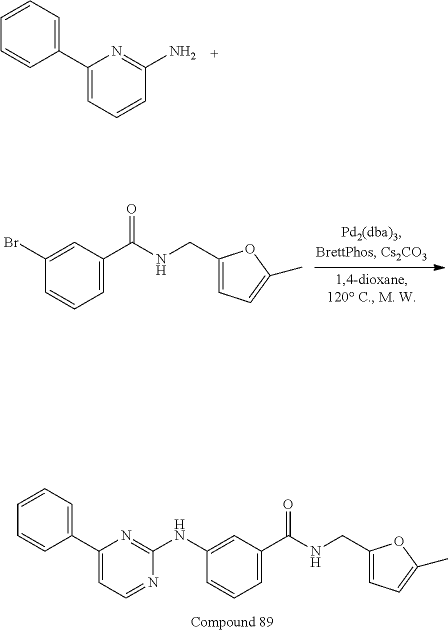 [SMILES: Nc1cccc(-c2ccccc2)n1 ; Cc1ccc(CNC(=O)c2cccc(Br)c2)o1 ; Cc1ccc(CNC(=O)c2cccc(Nc3nccc(-c4ccccc4)n3)c2)o1]

4-Phenylpyrimidin-2-amine (40 mg, 0.24 mmol), 3-bromo-N-((5-methylfuran-2-yl)methyl)benzamide (94 mg, 0.28 mmol), Pd₂(dba)₃ (30 mg, 0.024 mmol), BrettPhos (25 mg, 0.047 mmol), and cesium carbonate (154 mg, 0.47 mmol) were mixed in 1,4-dioxane (1.2 mL) and heated in a microwave reactor for 90 minutes at 120° C. The reaction mixture was concentrated and purified by MPLC. The crude mixture was solidified by using EA and HEX to give compound 89, N-[(5-methylfuran-2-yl)methyl]-3-[(4-phenylpyrimidin-2-yl)amino]benzamide (27 mg, 30%) as a beige solid.

Synthesis of Compound 90

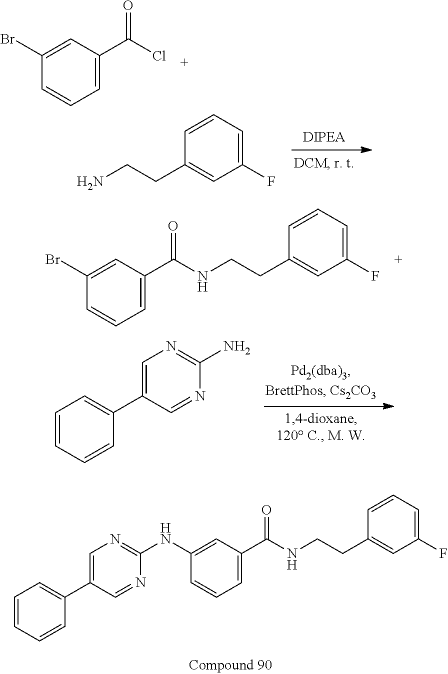

Step 1: 3-Bromobenzoyl chloride (0.12 mL, 0.91 mmol) and 2-(3-fluorophenyl)ethan-1-amine (0.12 mL, 0.91 mmol) were dissolved in DCM (9.1 mL), followed up by addition of DIPEA (0.34 mL, 1.96 mmol) and stirred for 22 hours at room temperature. The reaction mixture was extracted by EA and saturated aq. NH₄Cl. The organic layer was dried over anhydrous Na₂SO₄ and concentrated to give 3-bromo-N-(3-fluorophenethyl)benzamide (340 mg, >99%) as a yellow oil.

Step 2: 5-Phenylpyrimidin-2-amine (40 mg, 0.23 mmol), 3-bromo-N-(3-fluorophenethyl)benzamide (105 mg, 0.28 mmol), Pd₂(dba)₃ (29 mg, 0.023 mmol), BrettPhos (25 mg, 0.047 mmol), and cesium carbonate (152 mg, 0.47 mmol) were mixed in 1,4-dioxane (1.2 mL) and heated in a microwave reactor for 90 minutes at 120° C. The reaction mixture was concentrated and purified by MPLC. The crude mixture was solidified by using EA to give compound 90, N-[2-(3-fluorophenyl)ethyl]-3-[(5-phenylpyrimidin-2-yl)amino]benzamide (28 mg, 29%) as a white solid.

Synthesis of Compound 92

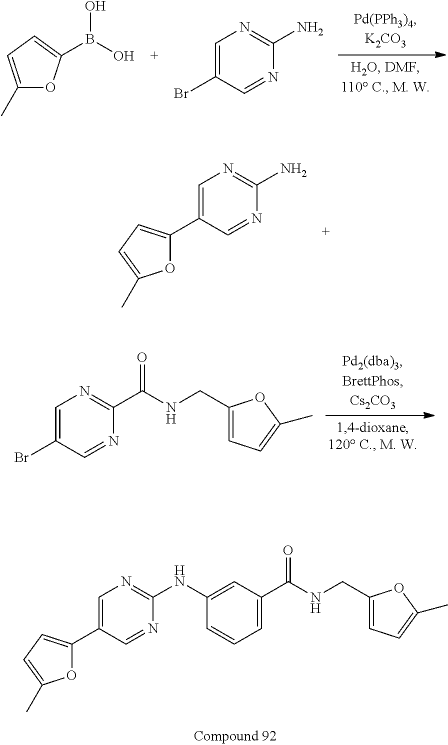

Step 1: (5-Methylfuran-2-yl)boronic acid (144 mg, 0.69 mmol), 5-bromopyrimidin-2-amine (100 mg, 0.57 mmol), Pd(PPh₃)₄ (33 mg, 0.03 mmol), and potassium carbonate (238 mg, 1.72 mmol) were mixed in H₂O/DMF (1.1/1.1 mL) and heated in a microwave reactor for 35 minutes at 110° C. The reaction mixture was extracted by EA and brine. The organic layer was dried over anhydrous Na₂SO₄ and concentrated. The residue was purified by MPLC to give 5-(5-methylfuran-2-yl)pyrimidin-2-amine (66 mg, 66%) as a yellowish white solid.

Step 2: 5-(5-Methylfuran-2-yl)pyrimidin-2-amine (60 mg, 0.17 mmol), 3-bromo-N-((5-methylfuran-2-yl)methyl)benzamide (60 mg, 0.21 mmol), Pd₂(dba)₃ (21 mg, 0.017 mmol), BrettPhos (18 mg, 0.034 mmol), and cesium carbonate (112 mg, 0.34 mmol) were mixed in 1,4-dioxane (0.86 mL) and heated in a microwave reactor for 90 minutes at 120° C. The reaction mixture was concentrated and purified by MPLC. The crude mixture was solidified by using EA and HEX to give compound 92, N-[(5-methylfuran-2-yl)methyl]-3-{[5-(5-methylfuran-2-yl)pyrimidin-2-yl]amino}benzamide (15 mg, 22%) as a beige solid.

Synthesis of Compound 98

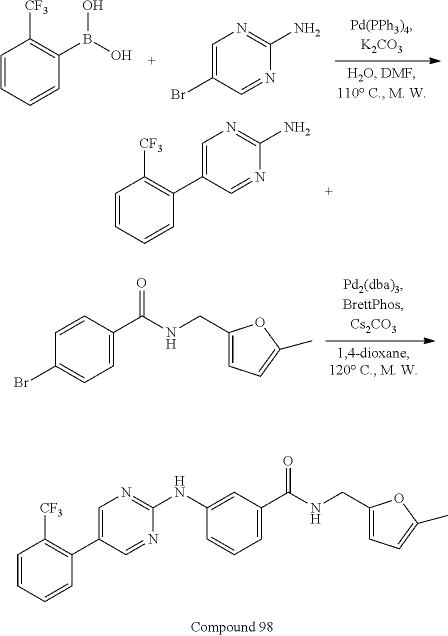

Step 1: (2-(Trifluoromethyl)phenyl)boronic acid (131 mg, 0.69 mmol), 5-bromopyrimidin-2-amine (100 mg, 0.57 mmol), Pd(PPh₃)₄ (33 mg, 0.03 mmol), and potassium carbonate (238 mg, 1.72 mmol) were mixed in H₂O/DMF (1.1/1.1 mL) and heated in a microwave reactor for 35 minutes at 110° C. The reaction mixture was extracted by EA and brine. The organic layer was dried over anhydrous Na₂SO₄ and concentrated. The residue was purified by MPLC to give 5-(2-(trifluoromethyl)phenyl)pyrimidin-2-amine (31 mg, 23%) as a yellow solid.

Step 2: 5-(2-(Trifluoromethyl)phenyl)pyrimidin-2-amine (30 mg, 0.13 mmol), 3-bromo-N-((5-methylfuran-2-yl)methyl)benzamide (44 mg, 0.15 mmol), Pd₂(dba)₃ (12 mg, 0.013 mmol), BrettPhos (14 mg, 0.025 mmol), and cesium carbonate (82 mg, 0.25 mmol) were mixed in 1,4-dioxane (0.63 mL) and heated in a microwave reactor for 90 minutes at 120° C. The reaction mixture was concentrated and purified by MPLC. The crude mixture was solidified by using EA to give compound 98, N-[(5-methylfuran-2-yl)methyl]-3-({5-[2-(trifluoromethyl)phenyl]pyrimidin-2-yl}amino)benzamide (17 mg, 31%) as a white solid.

Synthesis of Compound 99

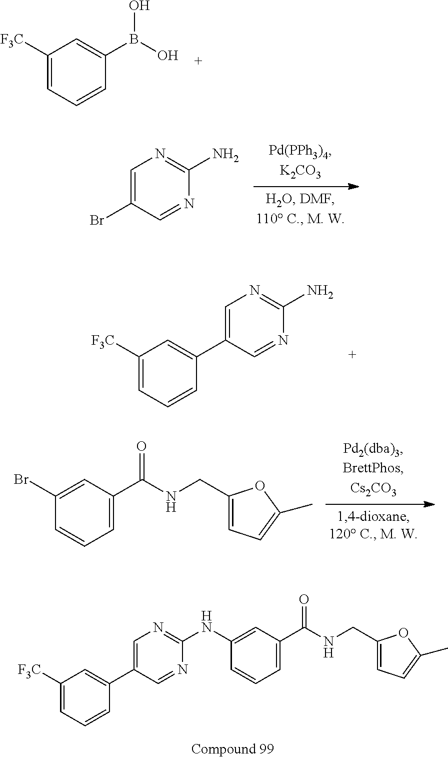 [SMILES: OB(O)c1cccc(C(F)(F)F)c1 ; Nc1ncc(Br)cn1 ; Nc1ncc(-c2cccc(C(F)(F)F)c2)cn1 ; Cc1ccc(CNC(=O)c2cccc(Br)c2)o1 ; Cc1ccc(CNC(=O)c2cccc(Nc3ncc(-c4cccc(C(F)(F)F)c4)cn3)c2)o1]

Step 1: (3-(Trifluoromethyl)phenyl)boronic acid (131 mg, 0.69 mmol), 5-bromopyrimidin-2-amine (100 mg, 0.57 mmol), Pd(PPh₃)₄ (33 mg, 0.03 mmol), and potassium carbonate (238 mg, 1.72 mmol) were mixed in H₂O/DMF (1.1/1.1 mL) and heated in a microwave reactor for 35 minutes at 110° C. The reaction mixture was extracted by EA and brine. The organic layer was dried over anhydrous Na₂SO₄ and concentrated. The crude mixture was solidified by using MeOH to give 5-(3-(trifluoromethyl)phenyl)pyrimidin-2-amine (54 mg, 40%) as a beige solid.

Step 2: 5-(3-(Trifluoromethyl)phenyl)pyrimidin-2-amine (40 mg, 0.17 mmol), 3-bromo-N-((5-methylfuran-2-yl)methyl)benzamide (59 mg, 0.2 mmol), Pd₂(dba)₃ (15 mg, 0.017 mmol), BrettPhos (18 mg, 0.034 mmol), and cesium carbonate (109 mg, 0.33 mmol) were mixed in 1,4-dioxane (0.84 mL) and heated in a microwave reactor for 90 minutes at 120° C. The reaction mixture was concentrated and purified by MPLC. The crude mixture was solidified by using EA to give compound 99, N-[(5-methylfuran-2-yl)methyl]-3-({5-[3-(trifluoromethyl)phenyl]pyrimidin-2-yl}amino)benzamide (21 mg, 28%) as a white solid.

Synthesis of Compound 100

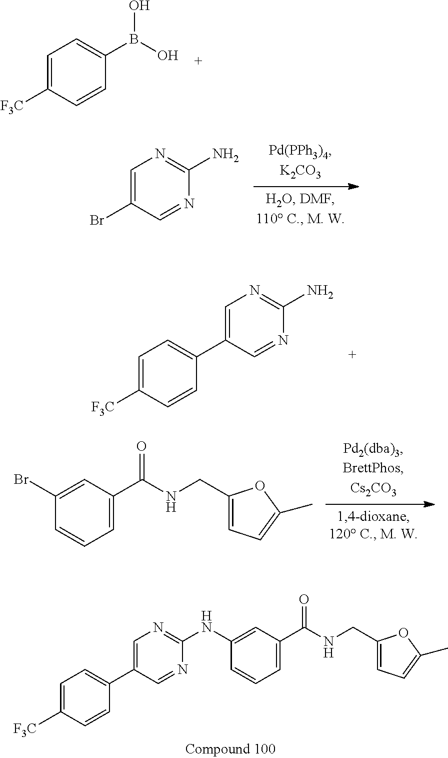 [SMILES: OB(O)c1ccc(C(F)(F)F)cc1 ; Nc1ncc(Br)cn1 ; Nc1ncc(-c2ccc(C(F)(F)F)cc2)cn1 ; Cc1ccc(CNC(=O)c2cccc(Br)c2)o1 ; Cc1ccc(CNC(=O)c2cccc(Nc3ncc(-c4ccc(C(F)(F)F)cc4)cn3)c2)o1]

Step 1: (4-(Trifluoromethyl)phenyl)boronic acid (131 mg, 0.69 mmol), 5-bromopyrimidin-2-amine (100 mg, 0.57 mmol), Pd(PPh₃)₄ (33 mg, 0.03 mmol), and potassium carbonate (238 mg, 1.72 mmol) were mixed in H₂O/DMF (1.1/1.1 mL) and heated in a microwave reactor for 35 minutes at 110° C. The reaction mixture was extracted by EA and brine. The organic layer was dried over anhydrous Na₂SO₄ and concentrated. The crude mixture was solidified by using MeOH to give 5-(4-(trifluoromethyl)phenyl)pyrimidin-2-amine (49 mg, 36%) as a beige solid.

Step 2: 5-(4-(Trifluoromethyl)phenyl)pyrimidin-2-amine (40 mg, 0.17 mmol), 3-bromo-N-((5-methylfuran-2-yl)methyl)benzamide (59 mg, 0.2 mmol), Pd₂(dba)₃ (15 mg, 0.017 mmol), BrettPhos (18 mg, 0.034 mmol), and cesium carbonate (109 mg, 0.33 mmol) were mixed in 1,4-dioxane (0.84 mL) and heated in a microwave reactor for 90 minutes at 120° C. The reaction mixture was concentrated and purified by MPLC. The crude mixture was solidified by using EA to give compound 100, N-[(5-methylfuran-2-yl)methyl]-3-({5-[4-(trifluoromethyl)phenyl]pyrimidin-2-yl}amino)benzamide (21 mg, 27%) as a white solid.

Synthesis of Compound 101

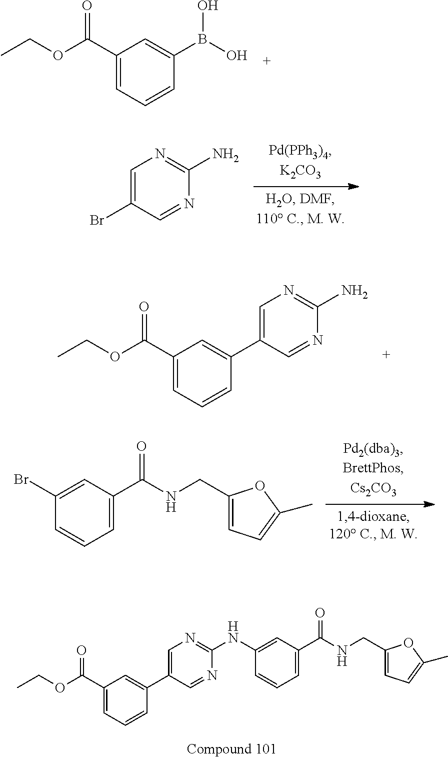

Step 1: (3-(Ethoxycarbonyl)phenyl)boronic acid (268 mg, 1.38 mmol), 5-bromopyrimidin-2-amine (200 mg, 1.15 mmol), Pd(PPh₃)₄ (66 mg, 0.06 mmol), and potassium carbonate (477 mg, 3.45 mmol) were mixed in H₂O/DMF (2.3/2.3 mL) and heated in a microwave reactor for 35 minutes at 110° C. The reaction mixture was extracted by EA and brine. The organic layer was dried over anhydrous Na₂SO₄ and concentrated. The crude mixture was solidified by using MeOH to give ethyl 3-(2-aminopyrimidin-5-yl)benzoate (130 mg, 47%) as a beige solid.

Step 2: Ethyl 3-(2-aminopyrimidin-5-yl)benzoate (100 mg, 0.41 mmol), 3-bromo-N-((5-methylfuran-2-yl)methyl)benzamide (145 mg, 0.49 mmol), Pd₂(dba)₃ (38 mg, 0.041 mmol), BrettPhos (44 mg, 0.082 mmol), and cesium carbonate (268 mg, 0.82 mmol) were mixed in 1,4-dioxane (2.1 mL) and heated in a microwave reactor for 90 minutes at 120° C. The reaction mixture was concentrated and purified by MPLC. The crude mixture was solidified by using EA to give compound 101, ethyl 3-{2-[(3-{[(5-methylfuran-2-yl)methyl]carbamoyl}phenyl)amino]pyrimidin-5-yl}benzoate (110 mg, 27%) as a white solid.

Synthesis of Compound 102

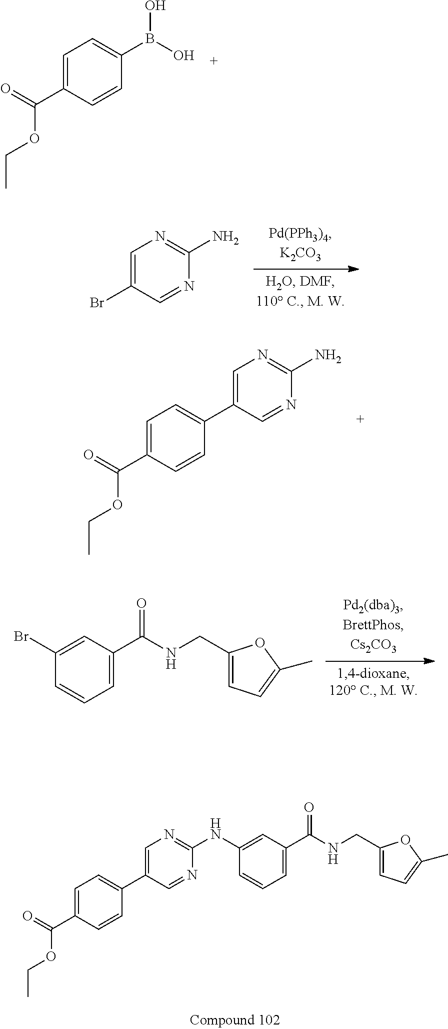 [SMILES: CCOC(=O)c1ccc(B(O)O)cc1 ; Nc1ncc(Br)cn1 ; CCOC(=O)c1ccc(-c2cnc(N)nc2)cc1 ; Cc1ccc(CNC(=O)c2cccc(Br)c2)o1 ; CCOC(=O)c1ccc(-c2cnc(Nc3cccc(C(=O)NCc4ccc(C)o4)c3)nc2)cc1]

Step 1: (4-(Ethoxycarbonyl)phenyl)boronic acid (268 mg, 1.38 mmol), 5-bromopyrimidin-2-amine (200 mg, 1.15 mmol), Pd(PPh₃)₄ (66 mg, 0.06 mmol), and potassium carbonate (477 mg, 3.45 mmol) were mixed in H₂O/DMF (2.3/2.3 mL) and heated in a microwave reactor for 35 minutes at 110° C. The reaction mixture was extracted by EA and brine. The organic layer was dried over anhydrous Na₂SO₄ and concentrated. The crude mixture was solidified by using MeOH to give ethyl 4-(2-aminopyrimidin-5-yl)benzoate (117 mg, 42%) as a beige solid.

Step 2: Ethyl 4-(2-aminopyrimidin-5-yl)benzoate (100 mg, 0.41 mmol), 3-bromo-N-((5-methylfuran-2-yl)methyl)benzamide (145 mg, 0.49 mmol), Pd₂(dba)₃ (38 mg, 0.041 mmol), BrettPhos (44 mg, 0.082 mmol), and cesium carbonate (268 mg, 0.82 mmol) were mixed in 1,4-dioxane (2.1 mL) and heated in a microwave reactor for 90 minutes at 120° C. The reaction mixture was concentrated and purified by MPLC. The crude mixture was solidified by using EA to give compound 102, ethyl 4-{2-[(3-{[(5-methylfuran-2-yl)methyl]carbamoyl}phenyl)amino]pyrimidin-5-yl}benzoate (96 mg, 23%) as a white solid.

Synthesis of Compound 103

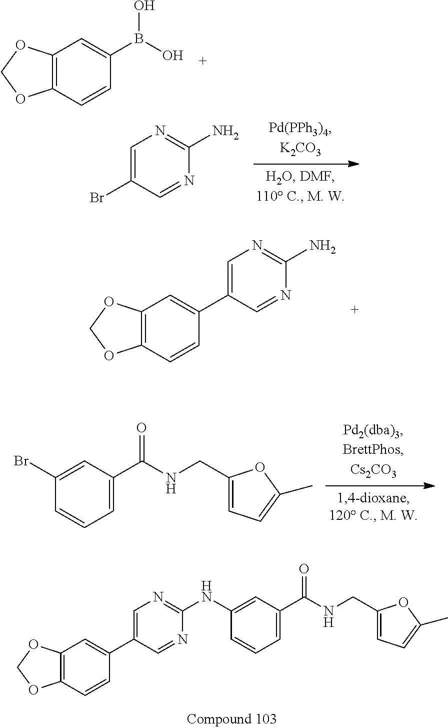 [SMILES: OB(O)c1ccc2c(c1)OCO2 ; Nc1ncc(Br)cn1 ; Nc1ncc(-c2ccc3c(c2)OCO3)cn1 ; Cc1ccc(CNC(=O)c2cccc(Br)c2)o1 ; Cc1ccc(CNC(=O)c2cccc(Nc3ncc(-c4ccc5c(c4)OCO5)cn3)c2)o1]

Step 1: Benzo[d][1,3]dioxol-5-ylboronic acid (171 mg, 0.69 mmol), 5-bromopyrimidin-2-amine (100 mg, 0.57 mmol), Pd(PPh₃)₄ (33 mg, 0.03 mmol), and potassium carbonate (238 mg, 1.72 mmol) were mixed in H₂O/DMF (1.1/1.1 mL) and heated in a microwave reactor for 35 minutes at 110° C. The reaction mixture was extracted by EA and brine. The organic layer was dried over anhydrous Na₂SO₄ and concentrated. The crude mixture was solidified by using MeOH to give 5-(benzo[d][1,3]dioxol-5-yl)pyrimidin-2-amine (72 mg, 58%) as a beige solid.

Step 2: 5-(Benzo[d][1,3]dioxol-5-yl)pyrimidin-2-amine (40 mg, 0.19 mmol), 3-bromo-N-((5-methylfuran-2-yl)methyl)benzamide (66 mg, 0.22 mmol), Pd₂(dba)₃ (17 mg, 0.019 mmol), BrettPhos (20 mg, 0.037 mmol), and cesium carbonate (121 mg, 0.37 mmol) were mixed in 1,4-dioxane (0.9 mL) and heated in a microwave reactor for 90 minutes at 120° C. The reaction mixture was concentrated and purified by MPLC. The crude mixture was solidified by using EA to give compound 103, 3-{[5-(2H-1,3-benzodioxol-5-yl)pyrimidin-2-yl]amino}-N-[(5-methylfuran-2-yl)methyl]benzamide (28 mg, 15%) as an orange solid.

Synthesis of Compound 104

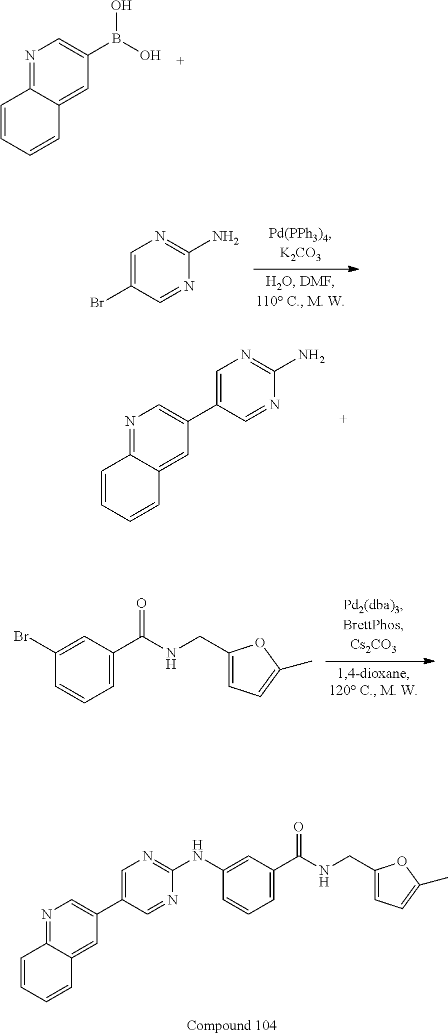 [SMILES: OB(O)c1cnc2ccccc2c1 ; Nc1ncc(Br)cn1 ; Nc1ncc(-c2cnc3ccccc3c2)cn1 ; Cc1ccc(CNC(=O)c2cccc(Br)c2)o1 ; Cc1ccc(CNC(=O)c2cccc(Nc3ncc(-c4cnc5ccccc5c4)cn3)c2)o1]

Step 1: Quinolin-3-ylboronic acid (119 mg, 0.69 mmol), 5-bromopyrimidin-2-amine (100 mg, 0.57 mmol), Pd(PPh₃)₄ (33 mg, 0.03 mmol), and potassium carbonate (238 mg, 1.72 mmol) were mixed in H₂O/DMF (1.1/1.1 mL) and heated in a microwave reactor for 35 minutes at 110° C. The reaction mixture was extracted by EA and brine. The organic layer was dried over anhydrous Na₂SO₄ and concentrated. The residue was purified by MPLC to give 5-(quinolin-3-yl)pyrimidin-2-amine (37 mg, 29%) as a white solid.

Step 2: 5-(Quinolin-3-yl)pyrimidin-2-amine (35 mg, 0.16 mmol), 3-bromo-N-((5-methylfuran-2-yl)methyl)benzamide (56 mg, 0.19 mmol), Pd₂(dba)₃ (14 mg, 0.016 mmol), BrettPhos (17 mg, 0.032 mmol), and cesium carbonate (103 mg, 0.31 mmol) were mixed in 1,4-dioxane (0.8 mL) and heated in a microwave reactor for 90 minutes at 120° C. The reaction mixture was concentrated and purified by MPLC. The crude mixture was solidified by using EA to give compound 104, N-[(5-methylfuran-2-yl)methyl]-3-{[5-(quinolin-3-yl)pyrimidin-2-yl]amino}benzamide (18 mg, 12%) as an orange solid.

Synthesis of Compound 107

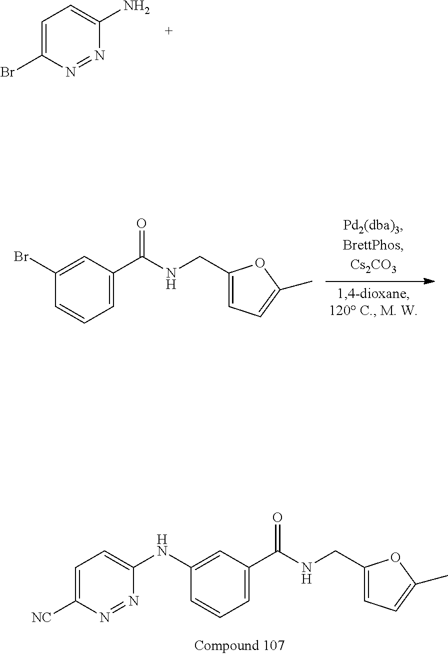 [SMILES: Nc1ccc(Br)nn1 ; Cc1ccc(CNC(=O)c2cccc(Br)c2)o1 ; Cc1ccc(CNC(=O)c2cccc(Nc3ccc(C#N)nn3)c2)o1]

6-Aminopyridazine-3-carbonitrile (40 mg, 0.33 mmol), 3-bromo-N-((5-methylfuran-2-yl)methyl)benzamide (118 mg, 0.4 mmol), Pd₂(dba)₃ (41 mg, 0.03 mmol), BrettPhos (36 mg, 0.07 mmol), and cesium carbonate (217 mg, 0.67 mmol) were mixed in 1,4-dioxane (1.7 mL) and heated in a microwave reactor for 90 minutes at 120° C. The reaction mixture was concentrated and purified by MPLC. The crude mixture was solidified by using EA to give compound 107, 3-[(6-cyanopyridazin-3-yl)amino]-N-[(5-methylfuran-2-yl)methyl]benzamide (27 mg, 24%) as a beige solid.

Synthesis of Compound 108

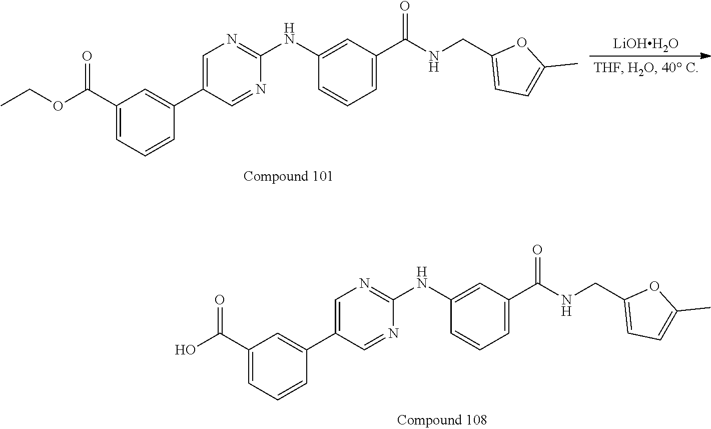

Ethyl 3-(2-((3-(((5-methylfuran-2-yl)methyl)carbamoyl)phenyl)amino)pyrimidin-5-yl)benzoate (32 mg, 0.07 mmol) and LiOH.H₂O (12 mg, 0.28 mmol) were mixed in THF/H₂O (0.47/0.23 mL) and stirred for 5 hours at 40° C. The reaction mixture was extracted by EA and aq. HCl (1N). The organic layer was dried over anhydrous Na₂SO₄ and concentrated. The crude mixture was solidified by using EA to give compound 108, 3-{2-[(3-{[(5-methylfuran-2-yl)methyl]carbamoyl}phenyl)amino]pyrimidin-5-yl}benzoic acid (21 mg, 70%) as a yellow solid.

Synthesis of Compound 109

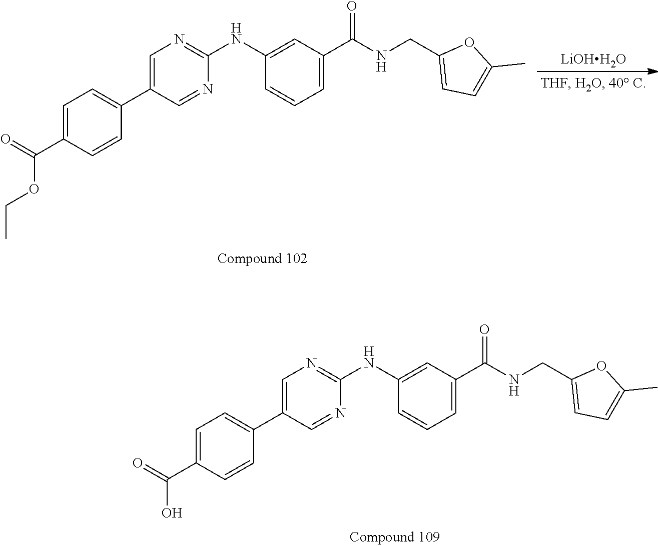

Ethyl 4-(2-((3-(((5-methylfuran-2-yl)methyl)carbamoyl)phenyl)amino)pyrimidin-5-yl)benzoate (32 mg, 0.07 mmol) and LiOH.H₂O (12 mg, 0.28 mmol) were mixed in THF/H₂O (0.47/0.23 mL) and stirred for 24 hours at 40° C. The reaction mixture was extracted by EA and aq. HCl (1N). The organic layer was dried over anhydrous Na₂SO₄ and concentrated. The crude mixture was solidified by using EA to give compound 109, 4-{2-[(3-{[(5-methylfuran-2-yl)methyl]carbamoyl}phenyl)amino]pyrimidin-5-yl}benzoic acid (20 mg, 66%) as a yellow solid.

Synthesis of Compound 110

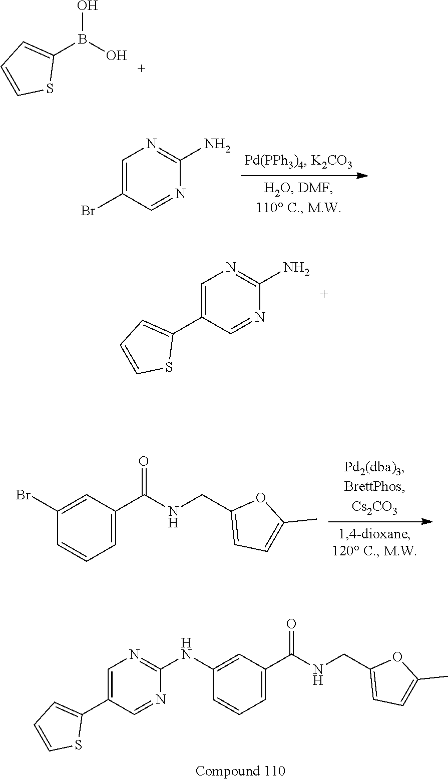

Step 1: Thiophen-2-ylboronic acid (132 mg, 1.03 mmol), 5-bromopyrimidin-2-amine (150 mg, 0.86 mmol), Pd(PPh₃)₄ (50 mg, 0.043 mmol) and potassium carbonate (357 mg, 2.59 mmol) were mixed in H₂O/DMF (1.7/1.7 mL) and heated in a microwave reactor for 35 minutes at 110° C. The reaction mixture was extracted by EA and brine. The organic layer was dried over anhydrous Na₂SO₄ and concentrated. The residue was purified by MPLC to give 5-(thiophen-2-yl)pyrimidin-2-amine (87 mg, 57%) as a beige solid.

Step 2: 5-(Thiophen-2-yl)pyrimidin-2-amine (70 mg, 0.24 mmol), 3-bromo-N-((5-methylfuran-2-yl)methyl)benzamide (84 mg, 0.28 mmol), Pd₂(dba)₃ (29 mg, 0.024 mmol), BrettPhos (25 mg, 0.047 mmol), and cesium carbonate (154 mg, 0.47 mmol) were mixed in 1,4-dioxane (1.2 mL) and heated in a microwave reactor for 90 minutes at 120° C. The reaction mixture was concentrated and purified by MPLC. The crude mixture was solidified by using EA to give compound 110, N-[(5-methylfuran-2-yl)methyl]-3-{[5-(thiophen-2-yl)pyrimidin-2-yl]amino}benzamide (34 mg, 36%) as a white solid.

Synthesis of Compound 111

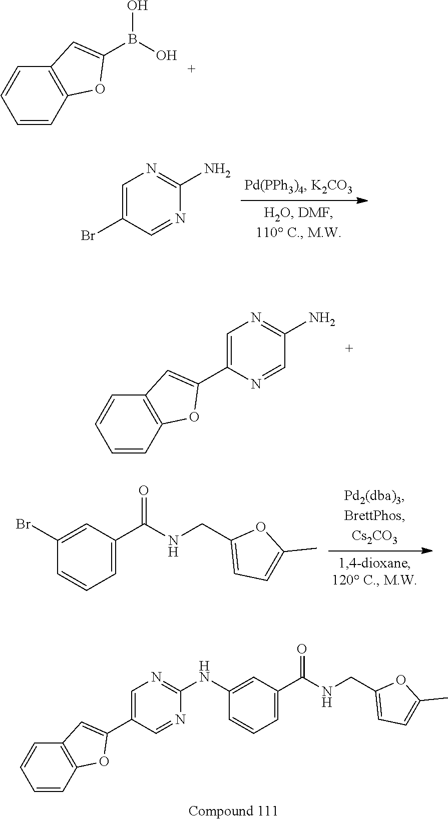

Step 1: Benzofuran-2-ylboronic acid (167 mg, 1.03 mmol), 5-bromopyrimidin-2-amine (150 mg, 0.86 mmol), Pd(PPh₃)₄ (50 mg, 0.043 mmol), and potassium carbonate (357 mg, 2.59 mmol) were mixed in H₂O/DMF (1.7/1.7 mL) and heated in a microwave reactor for 35 minutes at 110° C. The reaction mixture was extracted by EA and brine. The organic layer was dried over anhydrous Na₂SO₄ and concentrated. The crude mixture was solidified by using EA to give 5-(benzofuran-2-yl)pyrimidin-2-amine (67 mg, 37%) as a yellowish white solid.

Step 2: 5-(Benzofuran-2-yl)pyrimidin-2-amine (50 mg, 0.21 mmol), 3-bromo-N-((5-methylfuran-2-yl)methyl)benzamide (75 mg, 0.26 mmol), Pd₂(dba)₃ (26 mg, 0.021 mmol), BrettPhos (23 mg, 0.043 mmol), and cesium carbonate (154 mg, 0.47 mmol) were mixed in 1,4-dioxane (1.1 mL) and heated in a microwave reactor for 90 minutes at 120° C. The reaction mixture was concentrated and purified by MPLC. The crude mixture was solidified by using EA to give compound 111, 3-{[5-(1-benzofuran-2-yl)pyrimidin-2-yl]amino}-N-[(5-methylfuran-2-yl)methyl]benzamide (33 mg, 37%) as a white solid.

Synthesis of Compound 112

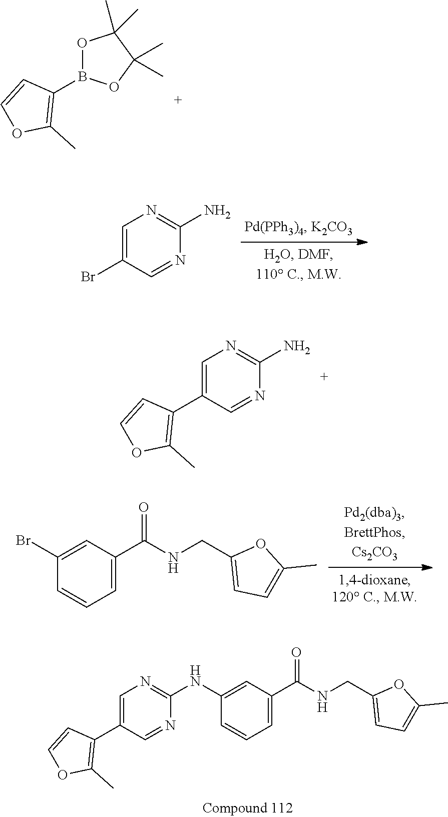

Step 1: 4,4,5,5-Tetramethyl-2-(2-methylfuran-3-yl)-1,3,2-dioxaborolane (167 mg, 1.03 mmol), 5-bromopyrimidin-2-amine (150 mg, 0.86 mmol), Pd(PPh₃)₄ (50 mg, 0.043 mmol), and potassium carbonate (357 mg, 2.59 mmol) were mixed in H₂O/DMF (1.7/1.7 mL) and heated in a microwave reactor for 35 minutes at 110° C. The reaction mixture was extracted by EA and brine. The organic layer was dried over anhydrous Na₂SO₄ and concentrated. The residue was purified by MPLC to give 5-(2-methylfuran-3-yl)pyrimidin-2-amine (154 mg, >99%) as a yellowish white solid.

Step 2: 5-(2-Methylfuran-3-yl)pyrimidin-2-amine (60 mg, 0.23 mmol), 3-bromo-N-((5-methylfuran-2-yl)methyl)benzamide (80 mg, 0.27 mmol), Pd₂(dba)₃ (28 mg, 0.023 mmol), BrettPhos (24 mg, 0.046 mmol), and cesium carbonate (148 mg, 0.45 mmol) were mixed in 1,4-dioxane (1.1 mL) and heated in a microwave reactor for 90 minutes at 120° C. The reaction mixture was concentrated and purified by MPLC. The crude mixture was solidified by using EA to give compound 112, N-[(5-methylfuran-2-yl)methyl]-3-{[5-(2-methylfuran-3-yl)pyrimidin-2-yl]amino}benzamide (6 mg, 6%) as a white solid.

Synthesis of Compound 113

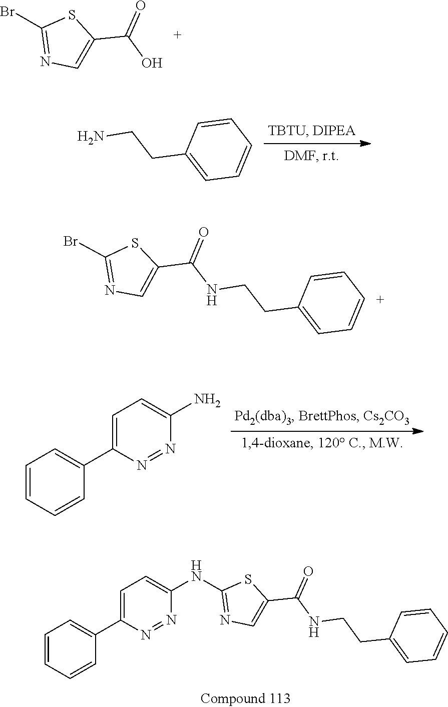

Step 1: 2-Bromothiazole-5-carboxylic acid (416 mg, 2 mmol), 2-phenylethan-1-amine (0.28 mL, 2.2 mmol), and O-(benzotriazol-1-yl)-N,N,N′,N′-tetramethyluronium tetrafluoroborate (1.2 g, 4 mmol) were dissolved in DMF (20 mL), followed up by addition of DIPEA (0.7 mL, 4 mmol) and stirred for 5 hours at room temperature. The reaction mixture was extracted by EA and 5% aq. LiCl. The organic layer was dried over anhydrous Na₂SO₄ and concentrated. The residue was purified by MPLC to give 2-bromo-N-phenethylthiazole-5-carboxamide (410 mg, 46%) as a white solid.

Step 2: 6-Phenylpyridazin-3-amine (30 mg, 0.18 mmol), 2-bromo-N-phenethylthiazole-5-carboxamide (65 mg, 0.21 mmol), Pd₂(dba)₃ (21 mg, 0.018 mmol), BrettPhos (19 mg, 0.035 mmol), and cesium carbonate (114 mg, 0.35 mmol) were mixed in 1,4-dioxane (1 mL) and heated in a microwave reactor for 90 minutes at 120° C. The reaction mixture was concentrated and purified by MPLC to give compound 113, N-(2-phenylethyl)-2-[(6-phenylpyridazin-3-yl)amino]-1,3-thiazole-5-carboxamide (9 mg, 6%) as a brown foam.

Synthesis of Compound 114

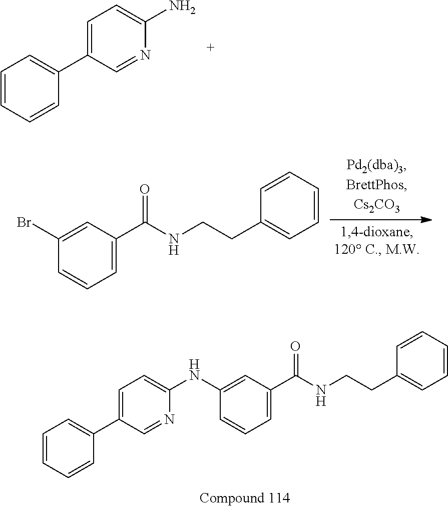 [SMILES: Nc1ccc(-c2ccccc2)cn1 ; "+" ; O=C(NCCc1ccccc1)c1cccc(Br)c1 ; O=C(NCCc1ccccc1)c1cccc(Nc2ccc(-c3ccccc3)cn2)c1]

5-Phenylpyridin-2-amine (40 mg, 0.24 mmol), 3-bromo-N-phenethylbenzamide (86 mg, 0.28 mmol), Pd₂(dba)₃ (22 mg, 0.024 mmol), BrettPhos (25 mg, 0.047 mmol), and cesium carbonate (153 mg, 0.47 mmol) were mixed in 1,4-dioxane (1.2 mL) and heated in a microwave reactor for 90 minutes at 120° C. The reaction mixture was concentrated and purified by MPLC. The crude mixture was solidified by using EA to give compound 114, N-(2-phenylethyl)-3-[(5-phenylpyridin-2-yl)amino]benzamide (18 mg, 20%) as a white solid.

Synthesis of Compound 115

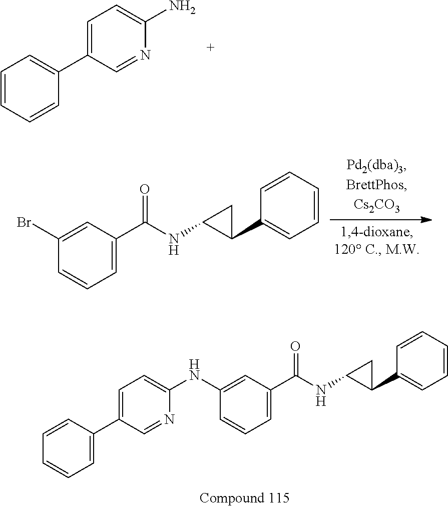 [SMILES: Nc1ccc(-c2ccccc2)cn1 ; "+" ; O=C(N[C@@H]1C[C@H]1c1ccccc1)c1cccc(Br)c1 ; O=C(N[C@@H]1C[C@H]1c1ccccc1)c1cccc(Nc2ccc(-c3ccccc3)cn2)c1]

5-Phenylpyridin-2-amine (40 mg, 0.24 mmol), 3-bromo-N-((1R,2S)-2-phenylcyclopropyl)benzamide (111 mg, 0.35 mmol), Pd₂(dba)₃ (22 mg, 0.024 mmol), BrettPhos (25 mg, 0.047 mmol), and cesium carbonate (153 mg, 0.47 mmol) were mixed in 1,4-dioxane (1.2 mL) and heated in a microwave reactor for 90 minutes at 120° C. The reaction mixture was concentrated and purified by MPLC. The crude mixture was solidified by using EA to give compound 115, N-[(1R,2S)-2-phenylcyclopropyl]-3-[(5-phenylpyridin-2-yl)amino]benzamide (26 mg, 27%) as a white solid.

2. Synthesis by Method B

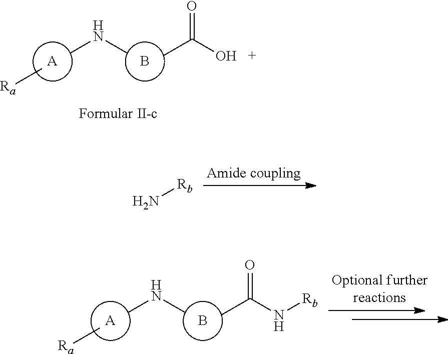

Synthesis of Compound 67

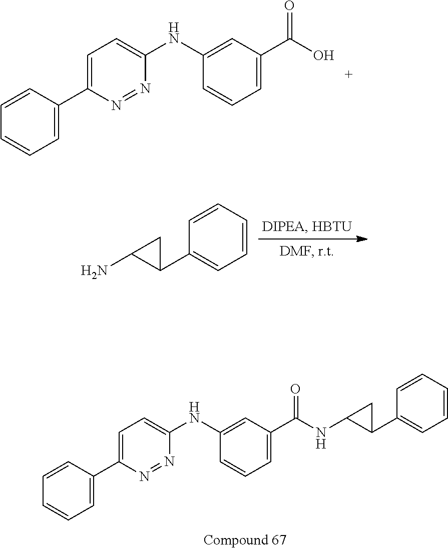

3-((6-Phenylpyridazin-3-yl)amino)benzoic acid (530 mg, 1.8 mmol), 2-phenylcyclopropan-1-amine (267 mg, 2 mmol) and hexafluorophosphate benzotriazole tetramethyl uronium (HBTU) (1 g, 2.7 mmol) were dissolved in DMF (18 mL), followed up by addition of DIPEA (0.95 mL, 5.5 mmol) and stirred for 18 hours at room temperature. The reaction mixture was extracted by EA and brine. The organic layer was dried over anhydrous Na₂SO₄ and concentrated. The residue was purified by MPLC. The crude mixture was solidified by using EA and HEX to give compound 67, N-(2-phenylcyclopropyl)-3-[(6-phenylpyridazin-3-yl)amino]benzamide (167 mg, 23%) as a beige solid.

Synthesis of Compound 68

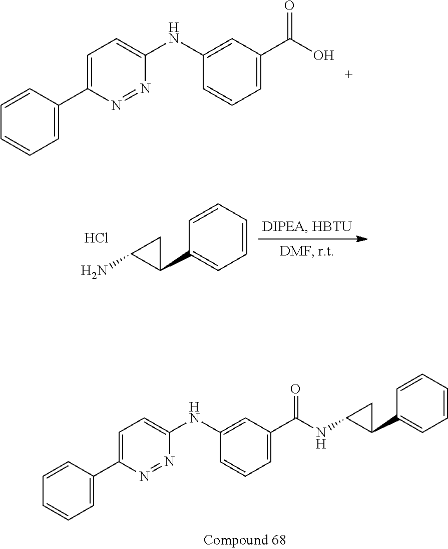

3-((6-Phenylpyridazin-3-yl)amino)benzoic acid (500 mg, 1.72 mmol), (1R,2S)-2-phenylcyclopropan-1-amine hydrochloride (320 mg, 1.89 mmol), and HBTU (976 mg, 2.57 mmol) were dissolved in DMF (17 mL), followed up by addition of DIPEA (0.9 mL, 5.2 mmol) and stirred for 20 hours at room temperature. The reaction mixture was extracted by EA and brine. The organic layer was dried over anhydrous Na₂SO₄ and concentrated. The reaction mixture was solidified by using EA and DCM to give compound 68, N-[(1R,2S)-2-phenylcyclopropyl]-3-[(6-phenylpyridazin-3-yl)amino]benzamide (376 mg, 54%) as a white solid.

Synthesis of Compound 79

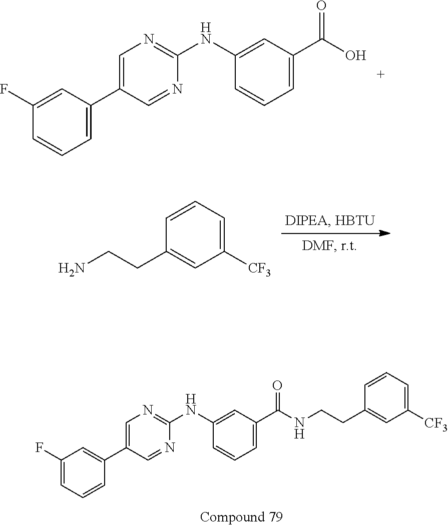

3-((5-(3-Fluorophenyl)pyrimidin-2-yl)amino)benzoic acid (550 mg, 1.78 mmol), 2-(3-(trifluoromethyl)phenyl)ethan-1-amine (308 mg, 1.96 mmol), and HBTU (1 g, 2.67 mmol) were dissolved in DMF (18 mL), followed up by addition of DIPEA (0.46 mL, 2.67 mmol) and stirred for 20 hours at room temperature. The reaction mixture was extracted by EA and brine. The organic layer was dried over anhydrous Na₂SO₄ and concentrated. The residue was solidified by using EA to give compound 79, 3-{[5-(3-fluorophenyl)pyrimidin-2-yl]amino}-N-{2-[3-(trifluoromethyl)phenyl]ethyl}benzamide (456 mg, 53%) as a white solid.

Synthesis of Compound 81

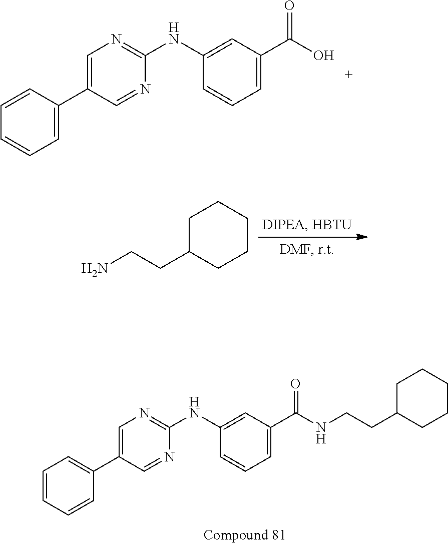

3-((5-Phenylpyrimidin-2-yl)amino)benzoic acid (600 mg, 2.06 mmol), 2-cyclohexylethan-1-amine (288 mg, 2.27 mmol), and HBTU (1.2 g, 3.09 mmol) were dissolved in DMF (21 mL), followed up by addition of DIPEA (0.54 mL, 3.09 mmol) and stirred for 20 hours at room temperature. The reaction mixture was extracted by EA and brine. The organic layer was dried over anhydrous Na₂SO₄ and concentrated. The residue was solidified by using EA to give compound 81, N-(2-cyclohexylethyl)-3-[(5-phenylpyrimidin-2-yl)amino]benzamide (395 mg, 48%) as a white solid.

Synthesis of Compound 93

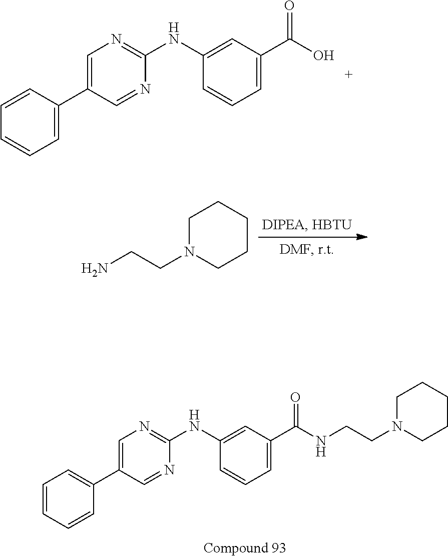

3-((5-Phenylpyrimidin-2-yl)amino)benzoic acid (30 mg, 0.1 mmol), 2-(piperidin-1-yl)ethan-1-amine (14.5 mg, 0.11 mmol), and HBTU (59 mg, 0.15 mmol) were dissolved in DMF (1 mL), followed up by addition of DIPEA (0.027 mL, 0.15 mmol) and stirred for 18 hours at room temperature. The reaction mixture was extracted by EA and saturated aq. NaHCO₃. The organic layer was dried over anhydrous Na₂SO₄ and concentrated. The residue was solidified by using EA and HEX to give compound 93, 3-[(5-phenylpyrimidin-2-yl)amino]-N-[2-(piperidin-1-yl)ethyl]benzamide (24 mg, 59%) as a beige solid.

Synthesis of Compound 94

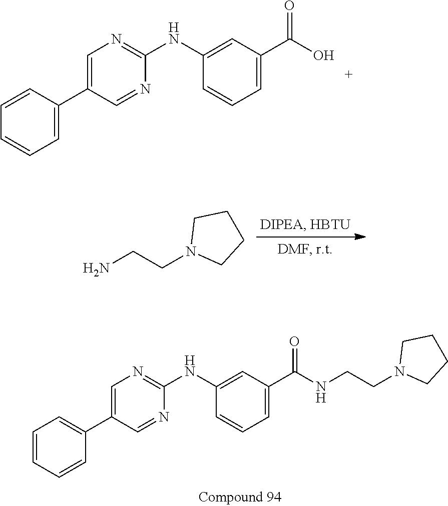

3-((5-Phenylpyrimidin-2-yl)amino)benzoic acid (30 mg, 0.1 mmol), 2-(pyrrolidin-1-yl)ethan-1-amine (13 mg, 0.11 mmol), and HBTU (59 mg, 0.15 mmol) were dissolved in DMF (1 mL), followed up by addition of DIPEA (0.027 mL, 0.15 mmol) and stirred for 18 hours at room temperature. The reaction mixture was extracted by EA and saturated aq. NaHCO₃. The organic layer was dried over anhydrous Na₂SO₄ and concentrated. The residue was solidified by using EA and HEX to give compound 94, 3-[(5-phenylpyrimidin-2-yl)amino]-N-[2-(pyrrolidin-1-yl)ethyl]benzamide (29 mg, 73%) as a beige solid.

Synthesis of Compound 95

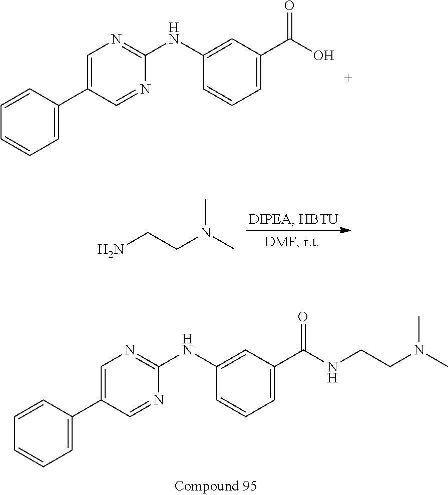

3-((5-Phenylpyrimidin-2-yl)amino)benzoic acid (30 mg, 0.1 mmol), N¹,N¹-dimethylethane-1,2-diamine (12 mg, 0.11 mmol), and HBTU (59 mg, 0.15 mmol) were dissolved in DMF (1 mL), followed up by addition of DIPEA (0.027 mL, 0.15 mmol) and stirred for 18 hours at room temperature. The reaction mixture was extracted by EA and saturated aq. NaHCO₃. The organic layer was dried over anhydrous Na₂SO₄ and concentrated. The residue was solidified by using EA and HEX to give compound 95, N-[2-(dimethylamino)ethyl]-3-[(5-phenylpyrimidin-2-yl)amino]benzamide (28 mg, 76%) as a white solid.

Synthesis of Compound 96

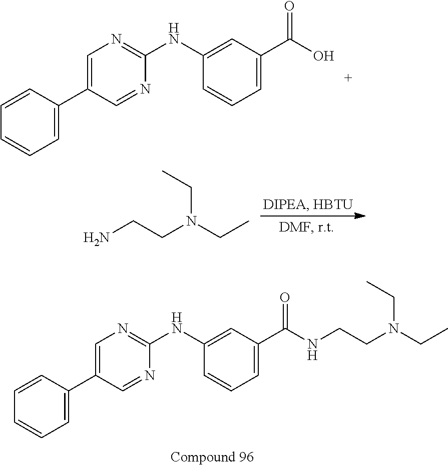

3-((5-Phenylpyrimidin-2-yl)amino)benzoic acid (30 mg, 0.1 mmol), N¹,N¹-diethylethane-1,2-diamine (13 mg, 0.11 mmol), and HBTU (59 mg, 0.15 mmol) were dissolved in DMF (1 mL), followed up by addition of DIPEA (0.027 mL, 0.15 mmol) and stirred for 18 hours at room temperature. The reaction mixture was extracted by EA and saturated aq. NaHCO₃. The organic layer was dried over anhydrous Na₂SO₄ and concentrated. The residue was solidified by using EA and HEX to give compound 96, N-[2-(diethylamino)ethyl]-3-[(5-phenylpyrimidin-2-yl)amino]benzamide (29 mg, 73%) as a white solid.

Synthesis of Compound 97

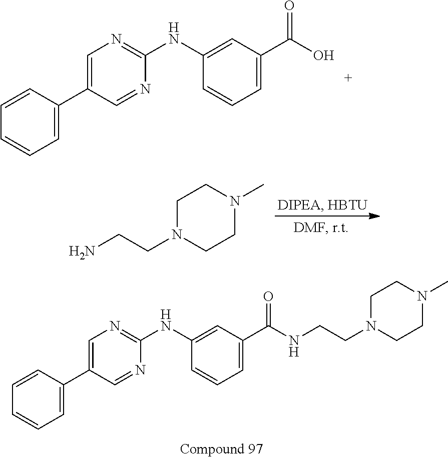

3-((5-Phenylpyrimidin-2-yl)amino)benzoic acid (30 mg, 0.1 mmol), 2-(4-methylpiperazin-1-yl)ethan-1-amine (16 mg, 0.11 mmol), and HBTU (59 mg, 0.15 mmol) were dissolved in DMF (1 mL), followed up by addition of DIPEA (0.027 mL, 0.15 mmol) and stirred for 18 hours at room temperature. The reaction mixture was extracted by EA and saturated aq. NaHCO₃. The organic layer was dried over anhydrous Na₂SO₄ and concentrated. The residue was solidified by using EA and HEX to give compound 97, N-[2-(4-methylpiperazin-1-yl)ethyl]-3-[(5-phenylpyrimidin-2-yl)amino]benzamide (36 mg, 83%) as a white solid.

Synthesis of Compound 105

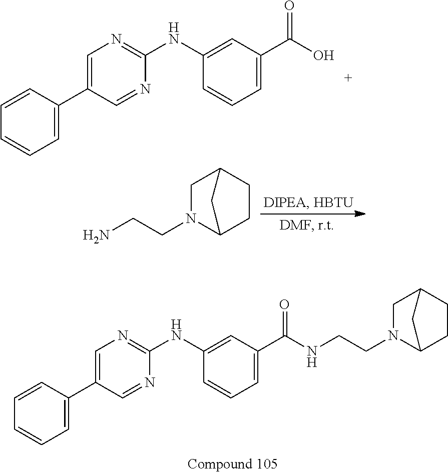 [SMILES: O=C(O)c1cccc(Nc2ncc(-c3ccccc3)cn2)c1 ; NCCN1CC2CCC1C2 ; O=C(NCCN1CC2CCC1C2)c1cccc(Nc2ncc(-c3ccccc3)cn2)c1]

3-((5-Phenylpyrimidin-2-yl)amino)benzoic acid (30 mg, 0.1 mmol), 2-(2-azabicyclo[2.2.1]heptan-2-yl)ethan-1-amine (16 mg, 0.11 mmol), and HBTU (59 mg, 0.15 mmol) were dissolved in DMF (1 mL), followed up by addition of DIPEA (0.027 mL, 0.15 mmol) and stirred for 18 hours at room temperature. The reaction mixture was extracted by EA and saturated aq. NaHCO₃. The organic layer was dried over anhydrous Na₂SO₄ and concentrated. The residue was solidified by using EA to give compound 105, N-(2-{2-azabicyclo[2.2.1]heptan-2-yl}ethyl)-3-[(5-phenylpyrimidin-2-yl)amino]benzamide (13 mg, 31%) as a beige solid.

Synthesis of Compound 106

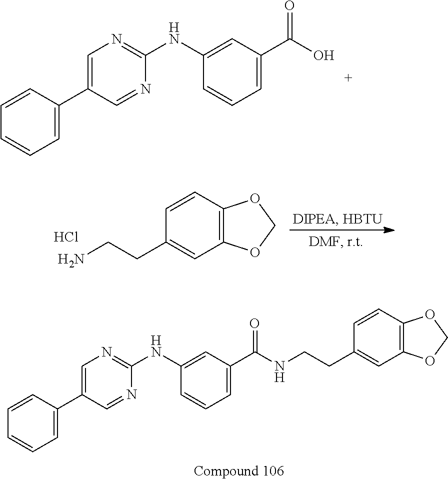 [SMILES: O=C(O)c1cccc(Nc2ncc(-c3ccccc3)cn2)c1 ; Cl ; NCCc1ccc2c(c1)OCO2 ; O=C(NCCc1ccc2c(c1)OCO2)c1cccc(Nc2ncc(-c3ccccc3)cn2)c1]

3-((5-Phenylpyrimidin-2-yl)amino)benzoic acid (30 mg, 0.1 mmol), 2-(benzo[d][1,3]dioxol-5-yl)ethan-1-amine hydrochloride (23 mg, 0.11 mmol), and HBTU (59 mg, 0.15 mmol) were dissolved in DMF (1 mL), followed up by addition of DIPEA (0.027 mL, 0.15 mmol) and stirred for 18 hours at room temperature. The reaction mixture was extracted by EA and saturated aq. NaHCO₃. The organic layer was dried over anhydrous Na₂SO₄ and concentrated. The residue was solidified by using EA to give compound 106, N-[2-(2H-1,3-benzodioxol-5-yl)ethyl]-3-[(5-phenylpyrimidin-2-yl)amino]benzamide (26 mg, 58%) as a beige solid.

Synthesis of Compound 116

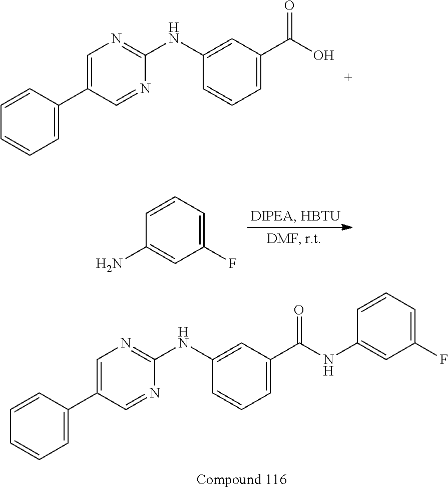 [SMILES: O=C(O)c1cccc(Nc2ncc(-c3ccccc3)cn2)c1 ; Nc1cccc(F)c1 ; O=C(Nc1cccc(F)c1)c1cccc(Nc2ncc(-c3ccccc3)cn2)c1]

3-((5-Phenylpyrimidin-2-yl)amino)benzoic acid (35 mg, 0.12 mmol), 3-fluoroaniline (15 mg, 0.13 mmol), and HBTU (68 mg, 0.18 mmol) were dissolved in DMF (1.2 mL), followed up by addition of DIPEA (0.03 mL, 0.18 mmol) and stirred for 18 hours at room temperature. The reaction mixture was extracted by EA and brine. The organic layer was dried over anhydrous Na₂SO₄ and concentrated. The residue was solidified by using EA and HEX to give compound 116, N-(3-fluorophenyl)-3-[(5-phenylpyrimidin-2-yl)amino]benzamide (32 mg, 69%) as a white solid.

Synthesis of Compound 117

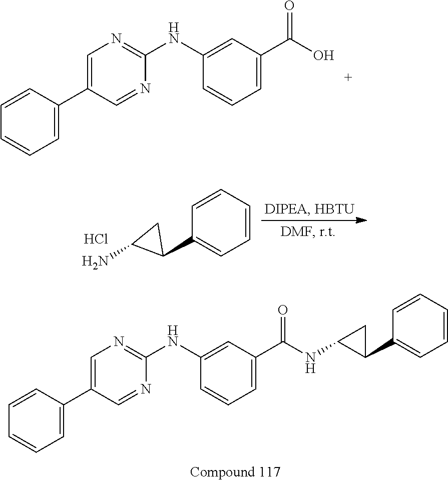 [SMILES: O=C(O)c1cccc(Nc2ncc(-c3ccccc3)cn2)c1 ; Cl ; N[C@@H]1C[C@H]1c1ccccc1 ; O=C(N[C@@H]1C[C@H]1c1ccccc1)c1cccc(Nc2ncc(-c3ccccc3)cn2)c1]

3-((5-Phenylpyrimidin-2-yl)amino)benzoic acid (35 mg, 0.12 mmol), (1R,2S)-2-phenylcyclopropan-1-amine hydrochloride (45 mg, 0.26 mmol), and HBTU (68 mg, 0.18 mmol) were dissolved in DMF (1.2 mL), followed up by addition of DIPEA (0.03 mL, 0.18 mmol) and stirred for 18 hours at room temperature. The reaction mixture was extracted by EA and brine. The organic layer was dried over anhydrous Na₂SO₄ and concentrated. The residue was solidified by using EA and HEX. The crude mixture was purified by MPLC to give compound 117, N-[(1R,2S)-2-phenylcyclopropyl]-3-[(5-phenylpyrimidin-2-yl)amino]benzamide (29 mg, 59%) as a white solid.

Synthesis of Compound 118

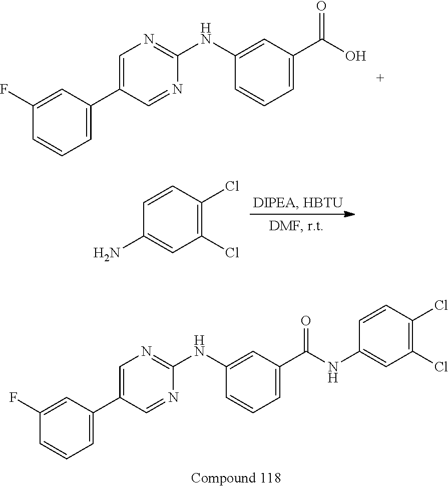 [SMILES: O=C(O)c1cccc(Nc2ncc(-c3cccc(F)c3)cn2)c1 ; Nc1ccc(Cl)c(Cl)c1 ; O=C(Nc1ccc(Cl)c(Cl)c1)c1cccc(Nc2ncc(-c3cccc(F)c3)cn2)c1]

3-((5-(3-Fluorophenyl)pyrimidin-2-yl)amino)benzoic acid (35 mg, 0.11 mmol), 3,4-dichloroaniline (20 mg, 0.12 mmol), and HBTU (64 mg, 0.17 mmol) were dissolved in DMF (1.1 mL), followed up by addition of DIPEA (0.03 mL, 0.17 mmol) and stirred for 18 hours at room temperature. The reaction mixture was extracted by EA and brine. The organic layer was dried over anhydrous Na₂SO₄ and concentrated. The residue was solidified by using EA and HEX to give compound 118, N-(3,4-dichlorophenyl)-3-{[5-(3-fluorophenyl)pyrimidin-2-yl]amino}benzamide (18 mg, 35%) as a beige solid.

Synthesis of Compound 119

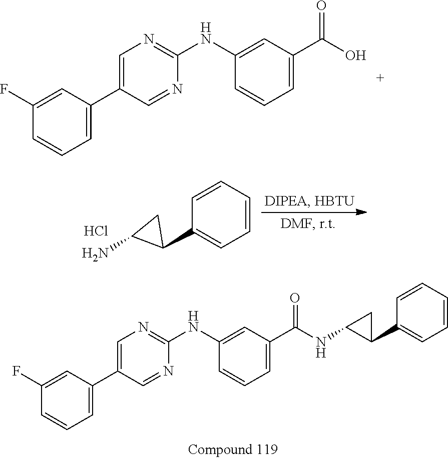 [SMILES: O=C(O)c1cccc(Nc2ncc(-c3cccc(F)c3)cn2)c1 ; Cl ; N[C@@H]1C[C@H]1c1ccccc1 ; O=C(N[C@@H]1C[C@H]1c1ccccc1)c1cccc(Nc2ncc(-c3cccc(F)c3)cn2)c1]

3-((5-(3-Fluorophenyl)pyrimidin-2-yl)amino)benzoic acid (35 mg, 0.11 mmol), (1R,2S)-2-phenylcyclopropan-1-amine hydrochloride (21 mg, 0.12 mmol), and HBTU (64 mg, 0.17 mmol) were dissolved in DMF (1.1 mL), followed up by addition of DIPEA (0.05 mL, 0.28 mmol) and stirred for 18 hours at room temperature. The reaction mixture was extracted by EA and brine. The organic layer was dried over anhydrous Na₂SO₄ and concentrated. The residue was solidified by using EA to give compound 119, 3-{[5-(3-fluorophenyl)pyrimidin-2-yl]amino}-N-[(1R,2S)-2-phenylcyclopropyl]benzamide (37 mg, 77%) as a white solid.

Synthesis of Compound 120

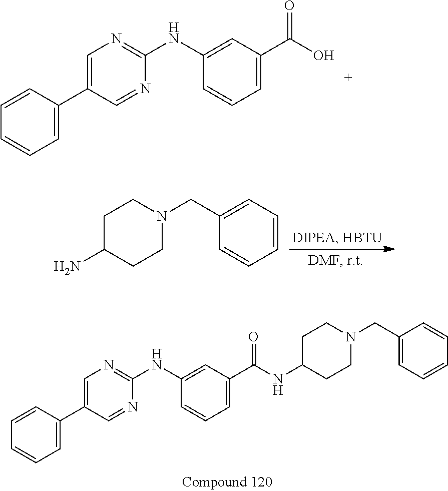 [SMILES: O=C(O)c1cccc(Nc2ncc(-c3ccccc3)cn2)c1 ; NC1CCN(Cc2ccccc2)CC1 ; O=C(NC1CCN(Cc2ccccc2)CC1)c1cccc(Nc2ncc(-c3ccccc3)cn2)c1]

3-((5-Phenylpyrimidin-2-yl)amino)benzoic acid (35 mg, 0.12 mmol), 1-benzylpiperidin-4-amine (25 mg, 0.13 mmol), and HBTU (68 mg, 0.18 mmol) were dissolved in DMF (1.2 mL), followed up by addition of DIPEA (0.03 mL, 0.18 mmol) and stirred for 18 hours at room temperature. The reaction mixture was extracted by EA and brine. The organic layer was dried over anhydrous Na₂SO₄ and concentrated. The residue was solidified by using EA and HEX to give compound 120, N-(1-benzylpiperidin-4-yl)-3-[(5-phenylpyrimidin-2-yl)amino]benzamide (34 mg, 62%) as a beige solid.

Synthesis of Compound 121

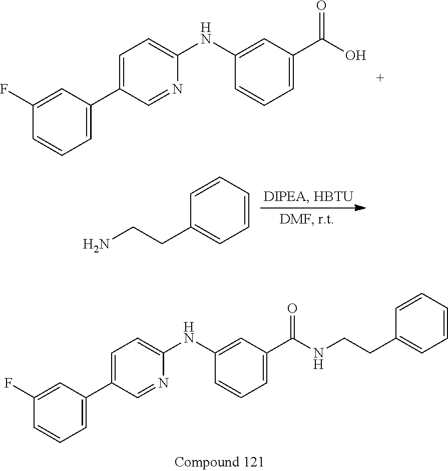 [SMILES: O=C(O)c1cccc(Nc2ccc(-c3cccc(F)c3)cn2)c1 ; NCCc1ccccc1 ; O=C(NCCc1ccccc1)c1cccc(Nc2ccc(-c3cccc(F)c3)cn2)c1]

3-((5-(3-Fluorophenyl)pyridin-2-yl)amino)benzoic acid (35 mg, 0.11 mmol), 2-phenylethan-1-amine (15 mg, 0.12 mmol), and HBTU (64 mg, 0.17 mmol) were dissolved in DMF (1.1 mL), followed up by addition of DIPEA (0.03 mL, 0.17 mmol) and stirred for 18 hours at room temperature. The reaction mixture was extracted by EA and brine. The organic layer was dried over anhydrous Na₂SO₄ and concentrated. The residue was solidified by using EA and HEX to give compound 121, 3-{[5-(3-fluorophenyl)pyridin-2-yl]amino}-N-(2-phenylethyl)benzamide (35 mg, 75%) as a white solid.

Synthesis of Compound 122

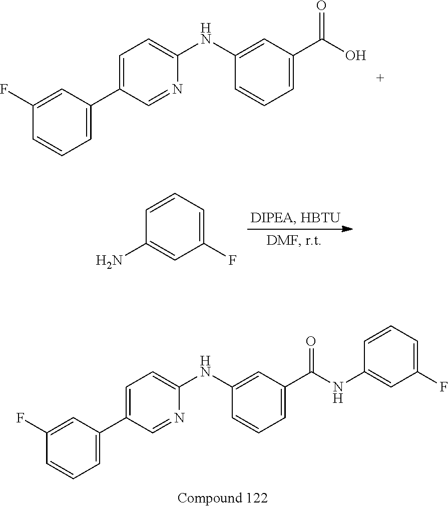 [SMILES: O=C(O)c1cccc(Nc2ccc(-c3cccc(F)c3)cn2)c1 ; Nc1cccc(F)c1 ; O=C(Nc1cccc(F)c1)c1cccc(Nc2ccc(-c3cccc(F)c3)cn2)c1]

3-((5-(3-Fluorophenyl)pyridin-2-yl)amino)benzoic acid (35 mg, 0.11 mmol), 3-fluoroaniline (14 mg, 0.12 mmol), and HBTU (64 mg, 0.17 mmol) were dissolved in DMF (1.1 mL), followed up by addition of DIPEA (0.03 mL, 0.17 mmol) and stirred for 18 hours at room temperature. The reaction mixture was extracted by EA and brine. The organic layer was dried over anhydrous Na₂SO₄ and concentrated. The residue was solidified by using EA and HEX to give compound 122, N-(3-fluorophenyl)-3-{[5-(3-fluorophenyl)pyridin-2-yl]amino}benzamide (20 mg, 44%) as a beige solid.

Synthesis of Compound 123

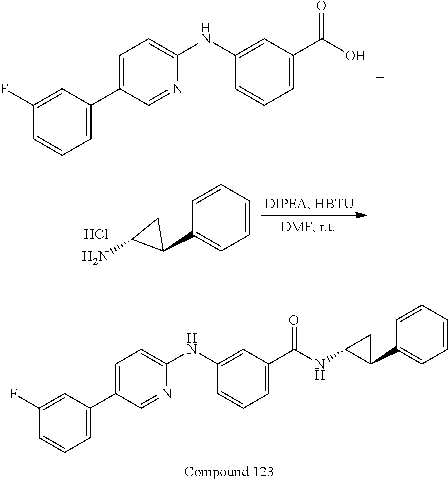 [SMILES: O=C(O)c1cccc(Nc2ccc(-c3cccc(F)c3)cn2)c1 ; Cl ; N[C@@H]1C[C@H]1c1ccccc1 ; O=C(N[C@@H]1C[C@H]1c1ccccc1)c1cccc(Nc2ccc(-c3cccc(F)c3)cn2)c1]

3-((5-(3-Fluorophenyl)pyridin-2-yl)amino)benzoic acid (35 mg, 0.11 mmol), (1R,2S)-2-phenylcyclopropan-1-amine hydrochloride (19 mg, 0.12 mmol), and HBTU (64 mg, 0.17 mmol) were dissolved in DMF (1.1 mL), followed up by addition of DIPEA (0.06 mL, 0.34 mmol) and stirred for 18 hours at room temperature. The reaction mixture was extracted by EA and brine. The organic layer was dried over anhydrous Na₂SO₄ and concentrated. The residue was solidified by using EA and HEX to give compound 123, 3-{[5-(3-fluorophenyl)pyridin-2-yl]amino}-N-[(1R,2S)-2-phenylcyclopropyl]benzamide (31 mg, 64%) as a white solid.

Synthesis of Compound 124

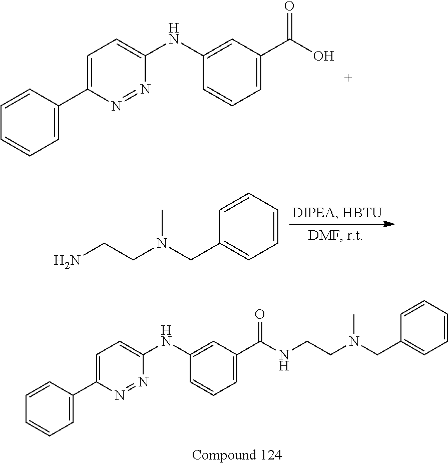 [SMILES: O=C(O)c1cccc(Nc2ccc(-c3ccccc3)nn2)c1 ; CN(CCN)Cc1ccccc1 ; CN(CCNC(=O)c1cccc(Nc2ccc(-c3ccccc3)nn2)c1)Cc1ccccc1]

3-((6-Phenylpyridazin-3-yl)amino)benzoic acid (35 mg, 0.12 mmol), N¹-benzyl-N¹-methylethane-1,2-diamine (22 mg, 0.13 mmol), and HBTU (68 mg, 0.18 mmol) were dissolved in DMF (1.2 mL), followed up by addition of DIPEA (0.03 mL, 0.18 mmol) and stirred for 18 hours at room temperature. The reaction mixture was extracted by EA and brine. The organic layer was dried over anhydrous Na₂SO₄ and concentrated. The residue was purified by MPLC. The crude mixture was solidified by using acetonitrile (ACN) to give compound 124, N-f2-[benzyl(methyl)amino]ethyl)-3-[(6-phenylpyridazin-3-yl)amino]benzamide (8 mg, 15%) as a white solid.

Synthesis of Compound 125

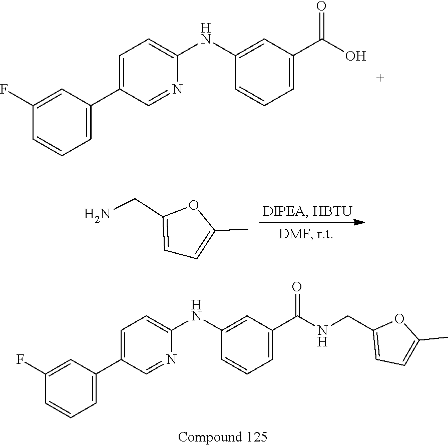

3-((5-(3-Fluorophenyl)pyridin-2-yl)amino)benzoic acid (60 mg, 0.19 mmol), (5-methylfuran-2-yl)methanamine (24 mg, 0.21 mmol), and HBTU (111 mg, 0.29 mmol) were dissolved in DMF (1.9 mL), followed up by addition of DIPEA (0.05 mL, 0.29 mmol) and stirred for 18 hours at room temperature. The reaction mixture was extracted by EA and brine. The organic layer was dried over anhydrous Na₂SO₄ and concentrated. The residue was solidified by using EA and HEX to give compound 125, 3-{[5-(3-fluorophenyl)pyridin-2-yl]amino}-N-[(5-methylfuran-2-yl)methyl]benzamide (46 mg, 59%) as a yellowish white solid.

Synthesis of Compound 126

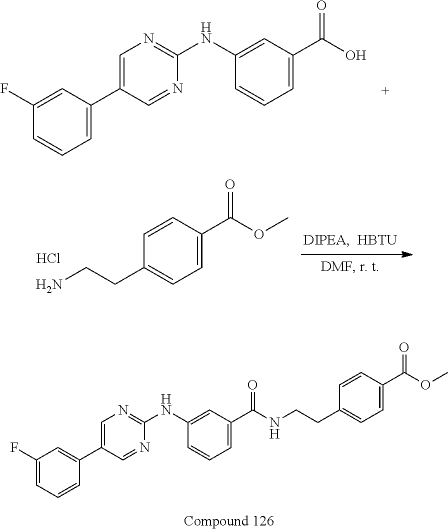 [SMILES: O=C(O)c1cccc(Nc2ncc(-c3cccc(F)c3)cn2)c1 ; Cl ; COC(=O)c1ccc(CCN)cc1 ; COC(=O)c1ccc(CCNC(=O)c2cccc(Nc3ncc(-c4cccc(F)c4)cn3)c2)cc1]

3-((5-(3-Fluorophenyl)pyrimidin-2-yl)amino)benzoic acid (200 mg, 0.65 mmol), methyl 4-(2-aminoethyl)benzoate hydrochloride (153 mg, 0.71 mmol), and HBTU (368 mg, 0.97 mmol) were dissolved in DMF (6.5 mL), followed up by addition of DIPEA (0.34 mL, 1.94 mmol) and stirred for 18 h at room temperature. The residue was solidified by using EA to give compound 126, methyl 4-(2-(3-((5-(3-fluorophenyl)pyrimidin-2-yl)amino)benzamido)ethyl)benzoate (242 mg, 92%) as a white solid.

Synthesis of Compound 127

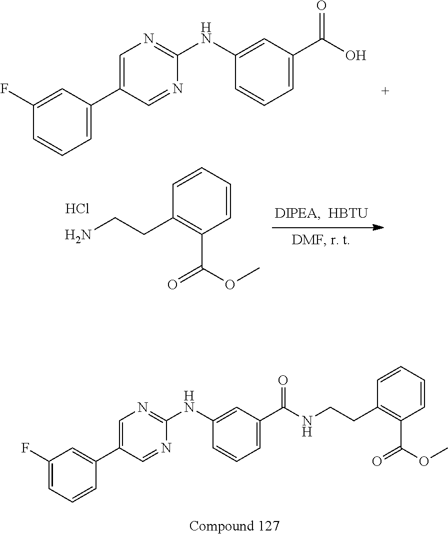 [SMILES: O=C(O)c1cccc(Nc2ncc(-c3cccc(F)c3)cn2)c1 ; Cl ; COC(=O)c1ccccc1CCN ; COC(=O)c1ccccc1CCNC(=O)c1cccc(Nc2ncc(-c3cccc(F)c3)cn2)c1]

3-((5-(3-Fluorophenyl)pyrimidin-2-yl)amino)benzoic acid (200 mg, 0.65 mmol), methyl 2-(2-aminoethyl)benzoate hydrochloride (153 mg, 0.71 mmol), and HBTU (368 mg, 0.97 mmol) were dissolved in DMF (6.5 mL), followed up by addition of DIPEA (0.34 mL, 1.94 mmol) and stirred for 18 h at room temperature. The reaction mixture was extracted by EA and brine. The white solid s precipitated out of the solution, and the solution was filtered to give compound 127, methyl 2-(2-(3-((5-(3-fluorophenyl)pyrimidin-2-yl)amino)benzamido)ethyl)benzoate (150 mg, 32%) as a white solid.

Synthesis of Compound 128

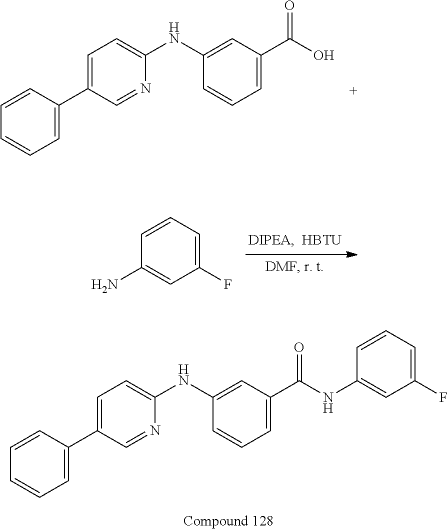 [SMILES: O=C(O)c1cccc(Nc2ccc(-c3ccccc3)cn2)c1 ; Nc1cccc(F)c1 ; O=C(Nc1cccc(F)c1)c1cccc(Nc2ccc(-c3ccccc3)cn2)c1]

3-((5-Phenylpyridin-2-yl)amino)benzoic acid (61 mg, 0.21 mmol), 3-fluoroaniline (26 mg, 0.23 mmol), and HBTU (119 mg, 0.32 mmol) were dissolved in DMF (2.1 mL), followed up by addition of DIPEA (0.055 mL, 0.32 mmol) and stirred for 18 h at room temperature. The reaction mixture was extracted by EA and brine. The organic layer was dried over anhydrous Na₂SO₄ and concentrated. The residue was solidified by using EA to give compound 128, N-(3-fluorophenyl)-3-((5-phenylpyridin-2-yl)amino)benzamide (38 mg, 47%) as a white solid.

Synthesis of Compound 129

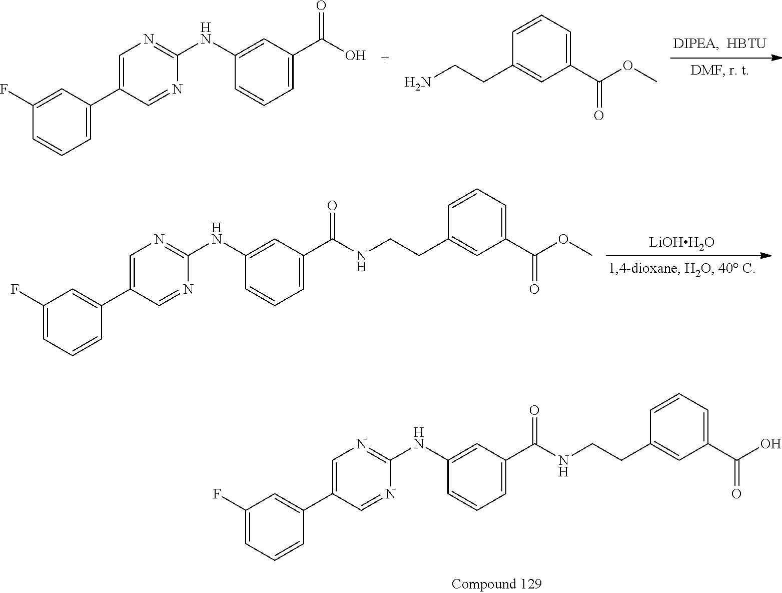 [SMILES: O=C(O)c1cccc(Nc2ncc(-c3cccc(F)c3)cn2)c1 ; COC(=O)c1cccc(CCN)c1 ; COC(=O)c1cccc(CCNC(=O)c2cccc(Nc3ncc(-c4cccc(F)c4)cn3)c2)c1 ; "LiOH·H2O" ; O=C(O)c1cccc(CCNC(=O)c2cccc(Nc3ncc(-c4cccc(F)c4)cn3)c2)c1]

Step 1: 3-((5-(3-Fluorophenyl)pyrimidin-2-yl)amino)benzoic acid (200 mg, 0.65 mmol), methyl 3-(2-aminoethyl)benzoate hydrochloride (153 mg, 0.71 mmol), and HBTU (368 g, 0.97 mmol) were dissolved in DMF (6.5 mL), followed up by addition of DIPEA (0.34 mL, 1.94 mmol) and stirred for 18 h at room temperature. The reaction mixture was extracted by EA and brine. The organic layer was dried over anhydrous Na₂SO₄ and concentrated. The residue was solidified by using EA to give methyl 3-(2-(3-((5-(3-fluorophenyl)pyrimidin-2-yl)amino)benzamido)ethyl)benzoate (281 mg, 107%) as a white solid.

Step 2: Methyl 3-(2-(3-((5-(3-fluorophenyl)pyrimidin-2-yl)amino)benzamido)ethyl)benzoate (100 mg, 0.21 mmol) and LiOH.H₂O (89.2 mg, 2.13 mmol) were mixed in H₂O/1,4-dioxane (0.89/4.25 mL) and stirred for 18 hours at 40° C. Then pH value of the solution was adjusted to 1-2 by 1 N HCl. The crude product was added into water. The suspension was filtered, and the filter cake was washed with water. The filter cake was dried under vacuum to give compound 129, 3-(2-(3-((5-(3-fluorophenyl)pyrimidin-2-yl)amino)benzamido)ethyl)benzoic acid (53 mg, 55%) as a yellowish white solid.

Synthesis of Compound 130

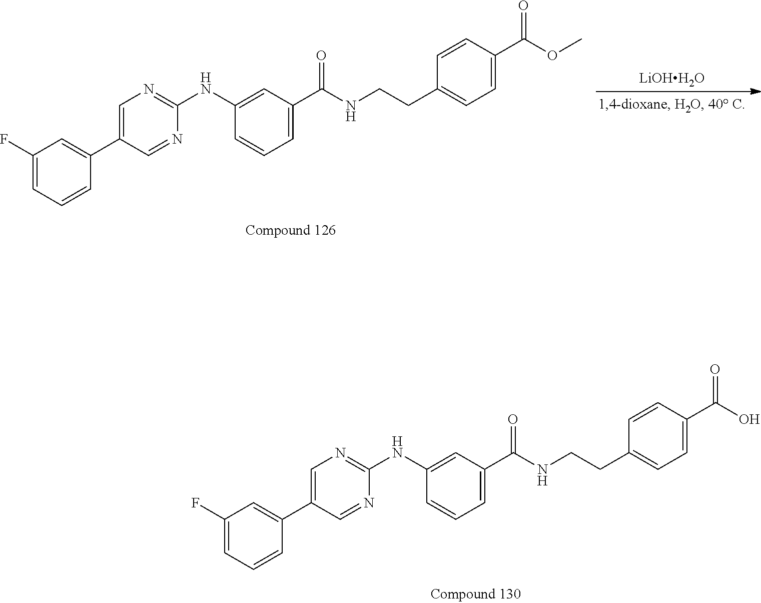 [SMILES: COC(=O)c1ccc(CCNC(=O)c2cccc(Nc3ncc(-c4cccc(F)c4)cn3)c2)cc1 ; "LiOH·H2O" ; O=C(O)c1ccc(CCNC(=O)c2cccc(Nc3ncc(-c4cccc(F)c4)cn3)c2)cc1]

Methyl 4-(2-(3-((5-(3-fluorophenyl)pyrimidin-2-yl)amino)benzamido)ethyl)benzoate (100 mg, 0.21 mmol) and LiOH.H₂O (89.2 mg, 2.13 mmol) were mixed in H₂O/1,4-dioxane (0.89/4.25 mL) and stirred for 42 hours at 40° C. Then pH value of the solution was adjusted to 1-2 by 1 N HCl. The crude product was added into water. The suspension was filtered, and the filter cake was washed with water. The crude product was added into EA. The suspension was filtered, and the filter cake was washed with EA. The filter cake was dried under vacuum to give compound 130, 4-(2-(3-((5-(3-fluorophenyl)pyrimidin-2-yl)amino)benzamido)ethyl)benzoic acid (84 mg, 87%) as a white solid.

Synthesis of Compound 131

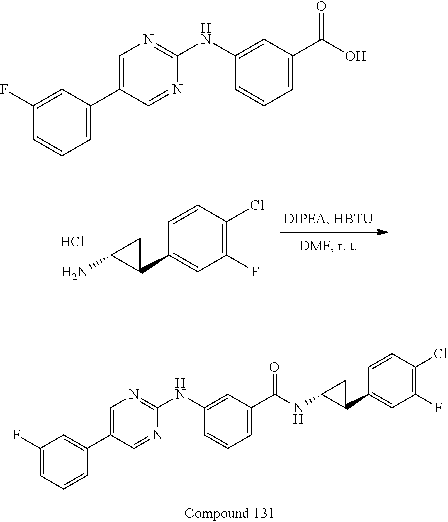

3-((5-(3-Fluorophenyl)pyrimidin-2-yl)amino)benzoic acid (150 mg, 0.48 mmol), (1R,2S)-2-(4-chloro-3-fluorophenyl)cyclopropan-1-amine hydrochloride (118 mg, 0.53 mmol) and HBTU (276 mg, 0.73 mmol) were dissolved in DMF (4.8 mL), followed up by addition of DIPEA (0.25 mL, 1.45 mmol) and stirred for 18 h at room temperature. The reaction mixture was extracted by EA and brine. The white solid was precipitated out of the solution, and the solution was filtered to give compound 131, N-((1R,2S)-2-(4-chloro-3-fluorophenyl)cyclopropyl)-3-((5-(3-fluorophenyl)pyrimidin-2-yl)amino)benzamide (72 mg, 31%) as a white solid.

Synthesis of Compound 132

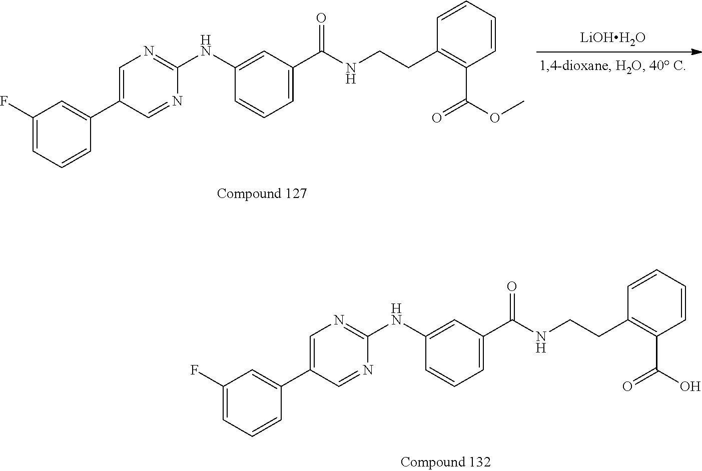 [SMILES: COC(=O)c1ccccc1CCNC(=O)c1cccc(Nc2ncc(-c3cccc(F)c3)cn2)c1 ; "LiOH·H2O" ; O=C(NCCc1ccccc1C(=O)O)c1cccc(Nc2ncc(-c3cccc(F)c3)cn2)c1]

Methyl 2-(2-(3-((5-(3-fluorophenyl)pyrimidin-2-yl)amino)benzamido)ethyl)benzoate (100 mg, 0.21 mmol) and LiOH.H₂O (89.2 mg, 2.13 mmol) were mixed in H₂O/1,4-dioxane (0.89/4.25 mL) and stirred for 42 hours at 40° C. The reaction mixture acidified by adding 1 N HCl and extracted by EA and brine. The organic layer was dried over anhydrous Na₂SO₄ and concentrated. The crude mixture was solidified by using EA to give compound 132, 2-(2-(3-((5-(3-fluorophenyl)pyrimidin-2-yl)amino)benzamido)ethyl)benzoic acid (80 mg, 82%) as a white solid.

Synthesis of Compound 133

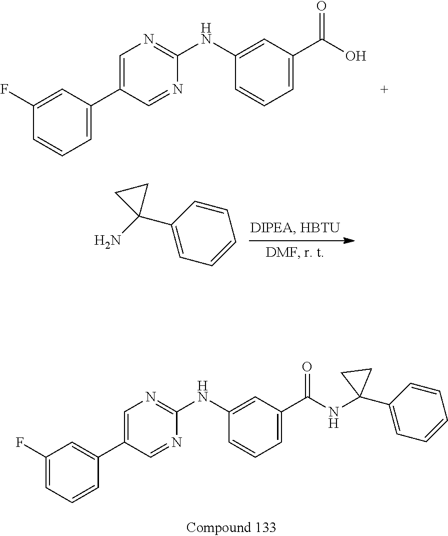 [SMILES: O=C(O)c1cccc(Nc2ncc(-c3cccc(F)c3)cn2)c1 ; NC1(c2ccccc2)CC1 ; O=C(NC1(c2ccccc2)CC1)c1cccc(Nc2ncc(-c3cccc(F)c3)cn2)c1]

3-((5-(3-Fluorophenyl)pyrimidin-2-yl)amino)benzoic acid (100 mg, 0.32 mmol), 1-phenylcyclopropan-1-amine (47 mg, 0.36 mmol), and HBTU (184 mg, 0.48 mmol) were dissolved in DMF (3.2 mL), followed up by addition of DIPEA (0.08 mL, 0.48 mmol) and stirred for 18 h at room temperature. The reaction mixture was extracted by EA and brine. The organic layer was dried over anhydrous Na₂SO₄ and concentrated. The crude mixture was solidified by using EA to give compound 133, 3-((5-(3-fluorophenyl)pyrimidin-2-yl)amino)-N-(1-phenylcyclopropyl)benzamide (83 mg, 60%) as a white solid.

Synthesis of Compound 134

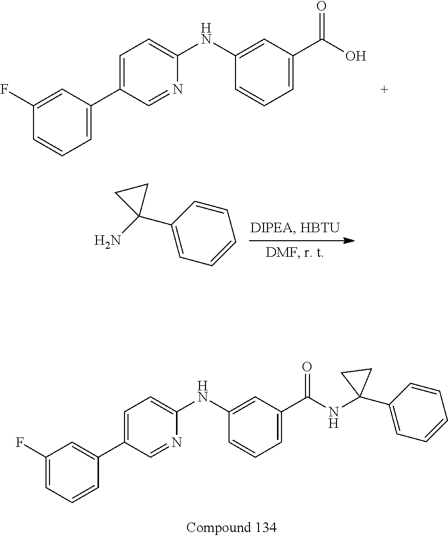 [SMILES: O=C(O)c1cccc(Nc2ccc(-c3cccc(F)c3)cn2)c1 ; NC1(c2ccccc2)CC1 ; O=C(NC1(c2ccccc2)CC1)c1cccc(Nc2ccc(-c3cccc(F)c3)cn2)c1]

3-((5-(3-Fluorophenyl)pyridin-2-yl)amino)benzoic acid (100 mg, 0.32 mmol), 1-phenylcyclopropan-1-amine (47 mg, 0.36 mmol), and HBTU (184 mg, 0.48 mmol) were dissolved in DMF (3.2 mL), followed up by addition of DIPEA (0.08 mL, 0.48 mmol) and stirred for 18 h at room temperature. The reaction mixture was extracted by EA and brine. The organic layer was dried over anhydrous Na₂SO₄ and concentrated. The crude mixture was solidified by using EA to give compound 134, 3-((5-(3-fluorophenyl)pyridin-2-yl)amino)-N-(1-phenylcyclopropyl)benzamide (93 mg, 68%) as a white solid.

Synthesis of Compound 135

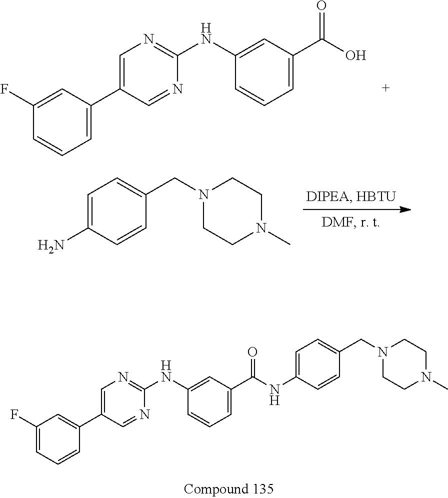 [SMILES: O=C(O)c1cccc(Nc2ncc(-c3cccc(F)c3)cn2)c1 ; CN1CCN(Cc2ccc(N)cc2)CC1 ; CN1CCN(Cc2ccc(NC(=O)c3cccc(Nc4ncc(-c5cccc(F)c5)cn4)c3)cc2)CC1]

3-((5-(3-Fluorophenyl)pyrimidin-2-yl)amino)benzoic acid (100 mg, 0.32 mmol), 4-((4-methylpiperazin-1-yl)methyl)aniline (73 mg, 0.36 mmol), and HBTU (184 mg, 0.48 mmol) were dissolved in DMF (3.2 mL), followed up by addition of DIPEA (0.08 mL, 0.48 mmol) and stirred for 18 h at room temperature. The reaction mixture was extracted by EA and saturated aq. NaHCO₃. The organic layer was dried over anhydrous Na₂SO₄ and concentrated. The crude mixture was solidified by using EA to give compound 135, 3-((5-(3-fluorophenyl)pyrimidin-2-yl)amino)-N-(4-((4-methylpiperazin-1-yl)methyl)phenyl)benzamide (116 mg, 73%) as a beige solid.

Synthesis of Compound 136

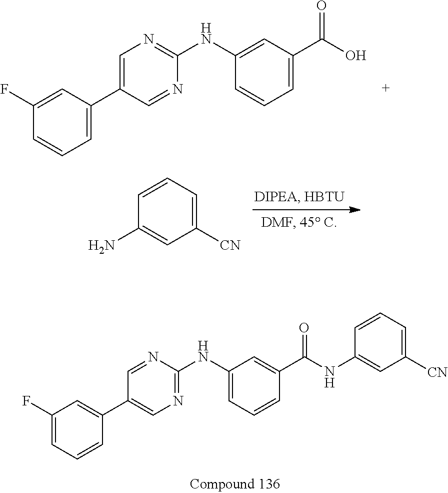 [SMILES: O=C(O)c1cccc(Nc2ncc(-c3cccc(F)c3)cn2)c1 ; N#Cc1cccc(N)c1 ; N#Cc1cccc(NC(=O)c2cccc(Nc3ncc(-c4cccc(F)c4)cn3)c2)c1]

3-((5-(3-Fluorophenyl)pyrimidin-2-yl)amino)benzoic acid (100 mg, 0.32 mmol), 3-aminobenzonitrile (42 mg, 0.36 mmol), and HBTU (184 mg, 0.48 mmol) were dissolved in DMF (3.2 mL), followed up by addition of DIPEA (0.08 mL, 0.48 mmol) and stirred for 3 days at 45° C. The reaction mixture was extracted by EA and brine. The beige solid was precipitated out of the solution, and the solution was filtered to give compound 136, N-(3-cyanophenyl)-3-((5-(3-fluorophenyl)pyrimidin-2-yl)amino)benzamide (56 mg, 42%) as a beige solid.

Synthesis of Compound 137

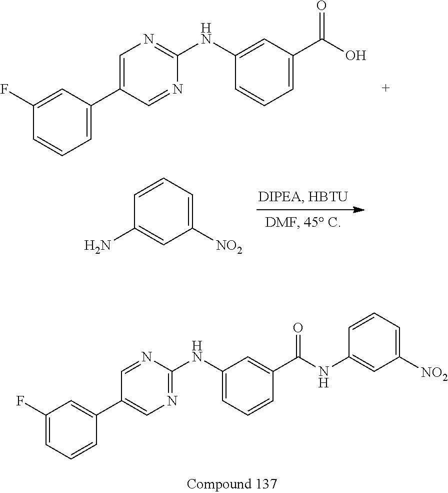 [SMILES: O=C(O)c1cccc(Nc2ncc(-c3cccc(F)c3)cn2)c1 ; Nc1cccc([N+](=O)[O-])c1 ; O=C(Nc1cccc([N+](=O)[O-])c1)c1cccc(Nc2ncc(-c3cccc(F)c3)cn2)c1]

3-((5-(3-Fluorophenyl)pyrimidin-2-yl)amino)benzoic acid (100 mg, 0.32 mmol), 3-nitroaniline (49 mg, 0.36 mmol), and HBTU (184 mg, 0.48 mmol) were dissolved in DMF (3.2 mL), followed up by addition of DIPEA (0.08 mL, 0.48 mmol) and stirred for 3 days at 45° C. The reaction mixture was extracted by EA and brine. The organic layer was dried over anhydrous Na₂SO₄ and concentrated. The crude mixture was solidified by using EA to give compound 137, 3-((5-(3-fluorophenyl)pyrimidin-2-yl)amino)-N-(3-nitrophenyl)benzamide (57 mg, 41%) as a yellow solid.

Synthesis of Compound 138

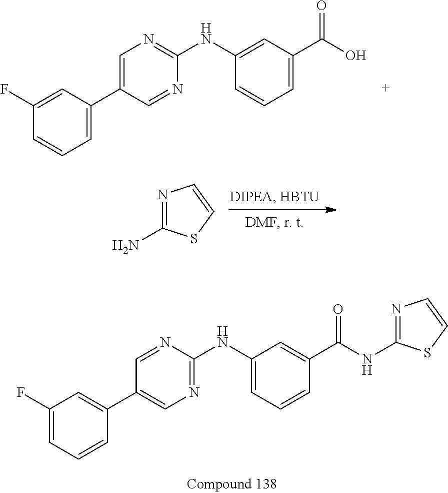 [SMILES: O=C(O)c1cccc(Nc2ncc(-c3cccc(F)c3)cn2)c1 ; Nc1nccs1 ; O=C(Nc1nccs1)c1cccc(Nc2ncc(-c3cccc(F)c3)cn2)c1]

3-((5-(3-Fluorophenyl)pyrimidin-2-yl)amino)benzoic acid (100 mg, 0.32 mmol), thiazol-2-amine (36 mg, 0.36 mmol), and HBTU (184 mg, 0.48 mmol) were dissolved in DMF (3.2 mL), followed up by addition of DIPEA (0.08 mL, 0.48 mmol) and stirred for 18 h at room temperature. The white solid was precipitated out of the solution. The crude product was added into EA, and the solution was filtered to give compound 138, 3-((5-(3-fluorophenyl)pyrimidin-2-yl)amino)-N-(thiazol-2-yl)benzamide (59 mg, 46%) as a white solid.

Synthesis of Compound 139

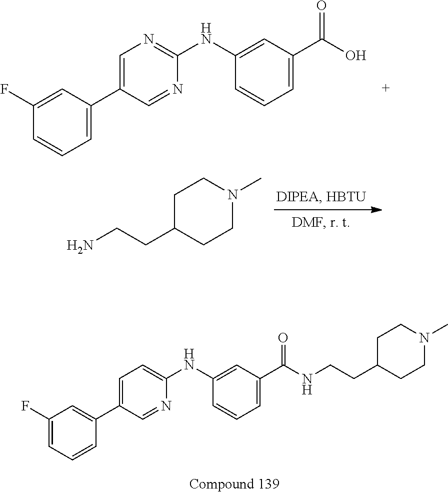

3-((5-(3-Fluorophenyl)pyridin-2-yl)amino)benzoic acid (100 mg, 0.32 mmol), 2-(1-methylpiperidin-4-yl)ethan-1-amine (51 mg, 0.36 mmol), and HBTU (184 mg, 0.48 mmol) were dissolved in DMF (3.2 mL), followed up by addition of DIPEA (0.08 mL, 0.49 mmol) and stirred for 18 h at room temperature. The reaction mixture was extracted by EA and brine. The organic layer was dried over anhydrous Na₂SO₄ and concentrated. The crude mixture was solidified by using EA and HEX to give compound 139, 3-((5-(3-fluorophenyl)pyridin-2-yl)amino)-N-(2-(1-methylpiperidin-4-yl)ethyl)benzamide (105 mg, 75%) as a yellowish white solid.

Synthesis of Compound 140

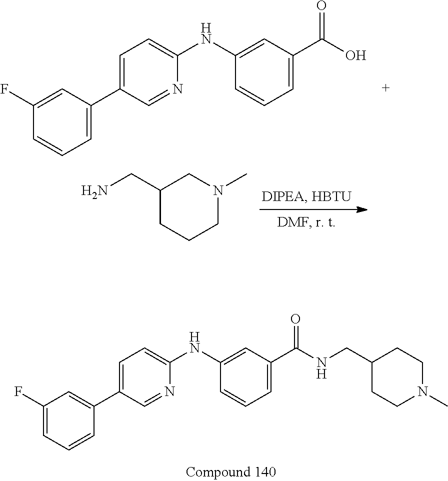

3-((5-(3-Fluorophenyl)pyridin-2-yl)amino)benzoic acid (100 mg, 0.32 mmol), (1-methylpiperidin-4-yl)methanamine (46 mg, 0.36 mmol), and HBTU (184 mg, 0.48 mmol) were dissolved in DMF (3.2 mL), followed up by addition of DIPEA (0.08 mL, 0.49 mmol) and stirred for 18 h at room temperature. The reaction mixture was extracted by EA and brine. The organic layer was dried over anhydrous Na₂SO₄ and concentrated. The crude mixture was solidified by using EA and HEX to give compound 140, 3-((5-(3-fluorophenyl)pyridin-2-yl)amino)-N-((1-methylpiperidin-4-yl)methyl)benzamide (34 mg, 25%) as a white solid.

Synthesis of Compound 141

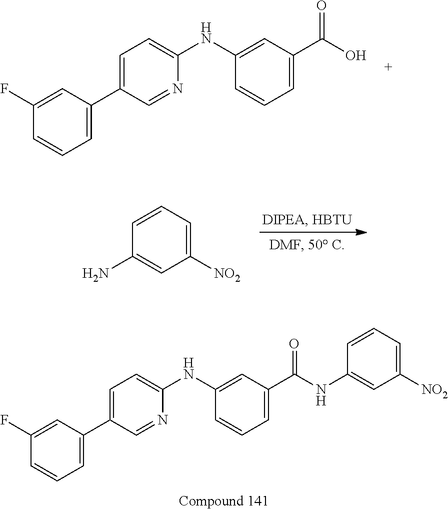 [SMILES: O=C(O)c1cccc(Nc2ccc(-c3cccc(F)c3)cn2)c1 ; Nc1cccc([N+](=O)[O-])c1 ; O=C(Nc1cccc([N+](=O)[O-])c1)c1cccc(Nc2ccc(-c3cccc(F)c3)cn2)c1]

3-((5-(3-Fluorophenyl)pyridin-2-yl)amino)benzoic acid (200 mg, 0.65 mmol), 3-nitroaniline (99 mg, 0.71 mmol), and HBTU (369 mg, 0.97 mmol) were dissolved in DMF (6.5 mL), followed up by addition of DIPEA (0.17 mL, 0.97 mmol) and stirred for 2 days at 50° C. The reaction mixture was extracted by EA and brine. The organic layer was dried over anhydrous Na₂SO₄ and concentrated. The crude mixture was solidified by using ACN to give compound 141, 3-((5-(3-fluorophenyl)pyridin-2-yl)amino)-N-(3-nitrophenyl)benzamide (49 mg, 16%) as a yellow solid.

Synthesis of Compound 142

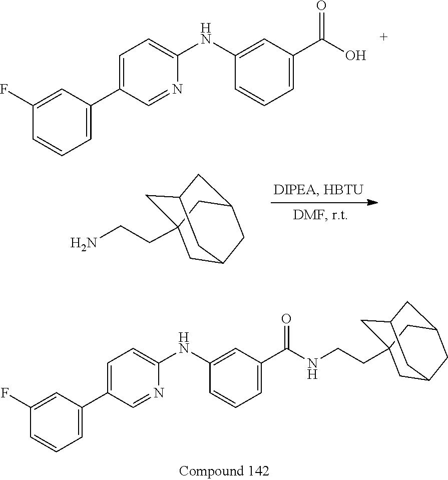 [SMILES: O=C(O)c1cccc(Nc2ccc(-c3cccc(F)c3)cn2)c1 ; NCCC12CC3CC(CC(C3)C1)C2 ; O=C(NCCC12CC3CC(CC(C3)C1)C2)c1cccc(Nc2ccc(-c3cccc(F)c3)cn2)c1]

3-((5-(3-Fluorophenyl)pyridin-2-yl)amino)benzoic acid (100 mg, 0.32 mmol), 1-(2-aminoethyl)adamantane (46 mg, 0.36 mmol), and HBTU (184 mg, 0.48 mmol) were dissolved in DMF (3.2 mL), followed up by addition of DIPEA (0.08 mL, 0.49 mmol) and stirred for 18 h at room temperature. The reaction mixture was extracted by EA and brine. The organic layer was dried over anhydrous Na₂SO₄ and concentrated. The crude mixture was solidified by using ACN to give compound 142, N-(2-(adamantan-1-yl)ethyl)-3-((5-(3-fluorophenyl)pyridin-2-yl)amino)benzamide (71 mg, 47%) as a beige solid.

Synthesis of Compound 143

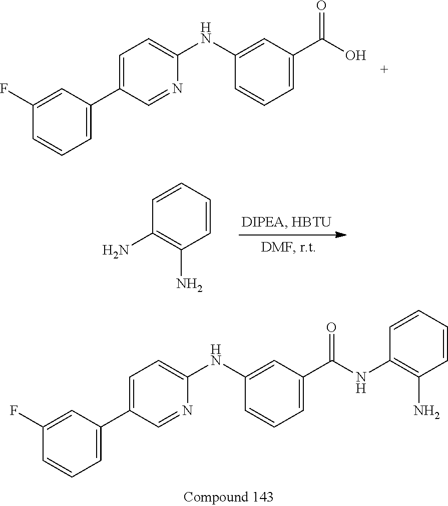 [SMILES: O=C(O)c1cccc(Nc2ccc(-c3cccc(F)c3)cn2)c1 ; Nc1ccccc1N ; Nc1ccccc1NC(=O)c1cccc(Nc2ccc(-c3cccc(F)c3)cn2)c1]

3-((5-(3-Fluorophenyl)pyridin-2-yl)amino)benzoic acid (100 mg, 0.32 mmol), benzene-1,2-diamine (39 mg, 0.36 mmol), and HBTU (184 mg, 0.48 mmol) were dissolved in DMF (3.2 mL), followed up by addition of DIPEA (0.08 mL, 0.49 mmol) and stirred for 18 h at room temperature. The reaction mixture was extracted by EA and brine. The organic layer was dried over anhydrous Na₂SO₄ and concentrated. The crude mixture was solidified by using EA to give compound 143, N-(2-aminophenyl)-3-((5-(3-fluorophenyl)pyridin-2-yl)amino)benzamide (72 mg, 56%) as a white solid.

Synthesis of Compound 144

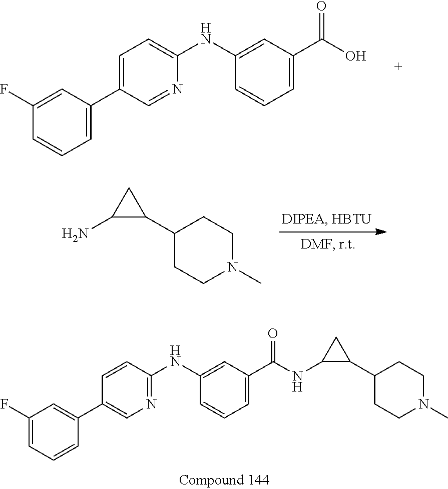 [SMILES: O=C(O)c1cccc(Nc2ccc(-c3cccc(F)c3)cn2)c1 ; CN1CCC(C2CC2N)CC1 ; CN1CCC(C2CC2NC(=O)c2cccc(Nc3ccc(-c4cccc(F)c4)cn3)c2)CC1]

3-((5-(3-Fluorophenyl)pyridin-2-yl)amino)benzoic acid (100 mg, 0.32 mmol), 2-(1-methylpiperidin-4-yl)cyclopropan-1-amine (55 mg, 0.36 mmol), and HBTU (184 mg, 0.48 mmol) were dissolved in DMF (3.2 mL), followed up by addition of DIPEA (0.08 mL, 0.49 mmol) and stirred for 18 h at room temperature. The reaction mixture was extracted by EA and brine. The organic layer was dried over anhydrous Na₂SO₄ and concentrated. The crude mixture was solidified by using EA and HEX to give compound 144, 3-((5-(3-fluorophenyl)pyridin-2-yl)amino)-N-(2-(1-methylpiperidin-4-yl)cyclopropyl)benzamide (10 mg, 7%) as a white solid.

Synthesis of Compound 145

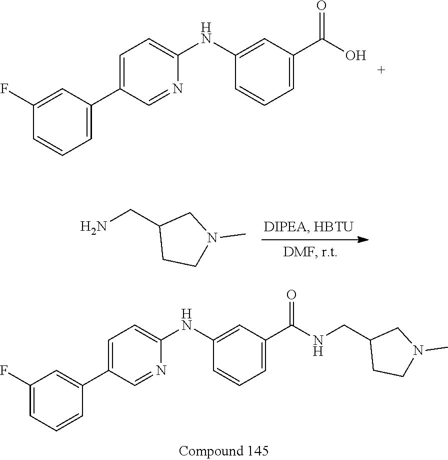

3-((5-(3-Fluorophenyl)pyridin-2-yl)amino)benzoic acid (100 mg, 0.32 mmol), (1-methylpyrrolidin-3-yl)methanamine (41 mg, 0.36 mmol), and HBTU (184 mg, 0.48 mmol) were dissolved in DMF (3.2 mL), followed up by addition of DIPEA (0.08 mL, 0.49 mmol) and stirred for 18 h at room temperature. The reaction mixture was extracted by EA and saturated aq. NaHCO₃. The organic layer was dried over anhydrous Na₂SO₄ and concentrated. The crude mixture was solidified by using DCM and HEX to give compound 145, 3-((5-(3-fluorophenyl)pyridin-2-yl)amino)-N-((1-methylpyrrolidin-3-yl)methyl)benzamide (19 mg, 15%) as a beige solid.

Synthesis of Compound 146

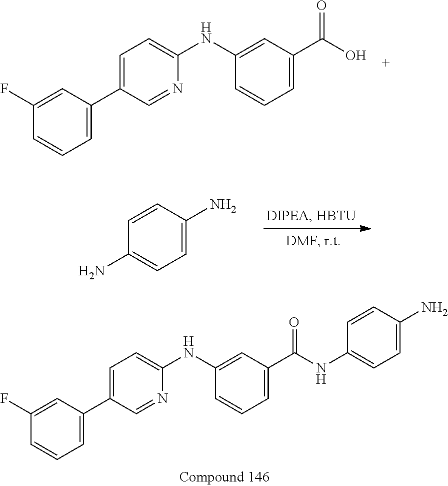 [SMILES: O=C(O)c1cccc(Nc2ccc(-c3cccc(F)c3)cn2)c1 ; Nc1ccc(N)cc1 ; Nc1ccc(NC(=O)c2cccc(Nc3ccc(-c4cccc(F)c4)cn3)c2)cc1]

3-((5-(3-Fluorophenyl)pyridin-2-yl)amino)benzoic acid (100 mg, 0.32 mmol), benzene-1,4-diamine (39 mg, 0.36 mmol), and HBTU (184 mg, 0.48 mmol) were dissolved in DMF (3.2 mL), followed up by addition of DIPEA (0.08 mL, 0.49 mmol) and stirred for 2 days at room temperature. The reaction mixture was extracted by EA and brine. The organic layer was dried over anhydrous Na₂SO₄ and concentrated. The crude mixture was solidified by using DCM and MeOH to give compound 146, N-(4-aminophenyl)-3-((5-(3-fluorophenyl)pyridin-2-yl)amino)benzamide (15 mg, 12%) as a beige solid.

Synthesis of Compound 147

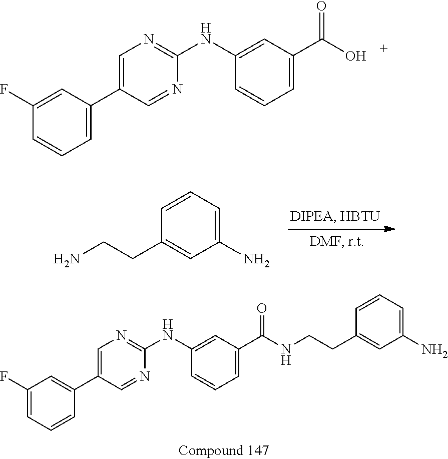 [SMILES: O=C(O)c1cccc(Nc2ncc(-c3cccc(F)c3)cn2)c1 ; NCCc1cccc(N)c1 ; Nc1cccc(CCNC(=O)c2cccc(Nc3ncc(-c4cccc(F)c4)cn3)c2)c1]

3-((5-(3-Fluorophenyl)pyrimidin-2-yl)amino)benzoic acid (100 mg, 0.32 mmol), 3-(2-aminoethyl)aniline (48 mg, 0.36 mmol), and HBTU (184 mg, 0.48 mmol) were dissolved in DMF (3.2 mL), followed up by addition of DIPEA (0.08 mL, 0.49 mmol) and stirred for 18 h at room temperature. The reaction mixture was extracted by EA and saturated aq. NaHCO₃. The organic layer was dried over anhydrous Na₂SO₄ and concentrated. The crude mixture was solidified by using EA and the solution was filtered. The filtrate was concentrated and solidified by using EA and HEX to give compound 147, N-(3-aminophenethyl)-3-((5-(3-fluorophenyl)pyrimidin-2-yl)amino)benzamide (36 mg, 26%) as a beige solid.

Synthesis of Compound 148

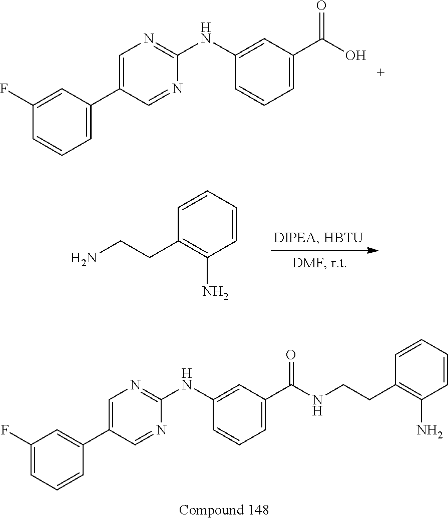 [SMILES: O=C(O)c1cccc(Nc2ncc(-c3cccc(F)c3)cn2)c1 ; NCCc1ccccc1N ; Nc1ccccc1CCNC(=O)c1cccc(Nc2ncc(-c3cccc(F)c3)cn2)c1]

3-((5-(3-Fluorophenyl)pyrimidin-2-yl)amino)benzoic acid (100 mg, 0.32 mmol), 2-(2-aminoethyl)aniline (48 mg, 0.36 mmol) and HBTU (184 mg, 0.48 mmol) were dissolved in DMF (3.2 mL), followed up by addition of DIPEA (0.08 mL, 0.49 mmol) and stirred for 18 h at room temperature. The reaction mixture was extracted by EA and saturated aq. NaHCO₃. The organic layer was dried over anhydrous Na₂SO₄ and concentrated. The crude mixture was solidified by using EA and the solution was filtered. The filtrate was concentrated and solidified by using EA and HEX to give compound 148, N-(2-aminophenethyl)-3-((5-(3-fluorophenyl)pyrimidin-2-yl)amino)benzamide (49 mg, 36%) as a beige solid.

Synthesis of Compound 149

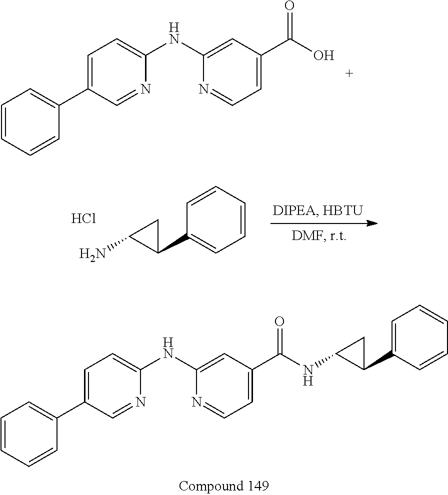 [SMILES: O=C(O)c1ccnc(Nc2ccc(-c3ccccc3)cn2)c1 ; Cl ; N[C@@H]1C[C@H]1c1ccccc1 ; O=C(N[C@@H]1C[C@H]1c1ccccc1)c1ccnc(Nc2ccc(-c3ccccc3)cn2)c1]

2-((5-Phenylpyridin-2-yl)amino)isonicotinic acid (60 mg, 0.21 mmol), (1R,2S)-2-phenylcyclopropan-1-amine hydrochloride (38 mg, 0.23 mmol), and HBTU (117 mg, 0.31 mmol) were dissolved in DMF (2.1 mL), followed up by addition of DIPEA (0.088 mL, 0.51 mmol) and stirred for 2 days at room temperature. The reaction mixture was extracted by EA and brine. The organic layer was dried over anhydrous Na₂SO₄ and concentrated. The residue was solidified by using EA to give compound 149, N-((1R,2S)-2-phenylcyclopropyl)-2-((5-phenylpyridin-2-yl)amino)isonicotinamide (51 mg, 60%) as a yellowish white solid.

Synthesis of Compound 150

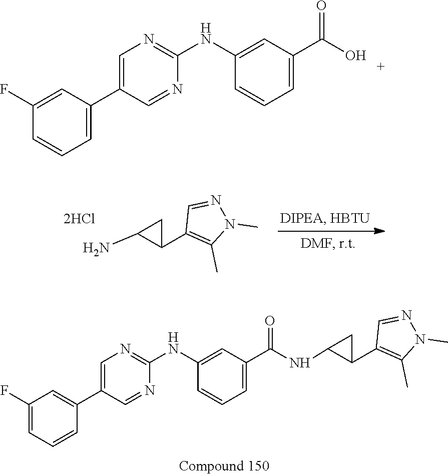 [SMILES: O=C(O)c1cccc(Nc2ncc(-c3cccc(F)c3)cn2)c1 ; Cc1c(C2CC2N)cnn1C ; Cc1c(C2CC2NC(=O)c2cccc(Nc3ncc(-c4cccc(F)c4)cn3)c2)cnn1C]

3-((5-(3-Fluorophenyl)pyrimidin-2-yl)amino)benzoic acid (100 mg, 0.32 mmol), 2-(1,5-dimethyl-1H-pyrazol-4-yl)cyclopropan-1-amine dihydrochloride (87 mg, 0.39 mmol) and HBTU (184 mg, 0.48 mmol) were dissolved in DMF (3.2 mL), followed up by addition of DIPEA (0.2 mL, 1.13 mmol) and stirred for 18 h at room temperature. The reaction mixture was extracted by EA and saturated aq. NaHCO₃. The organic layer was dried over anhydrous Na₂SO₄ and concentrated. The crude mixture was solidified by using EA to give compound 150, N-(2-(1,5-dimethyl-1H-pyrazol-4-yl)cyclopropyl)-3-((5-(3-fluorophenyl)pyrimidin-2-yl)amino)benzamide (102 mg, 71%) as a white solid.

Synthesis of Compound 151

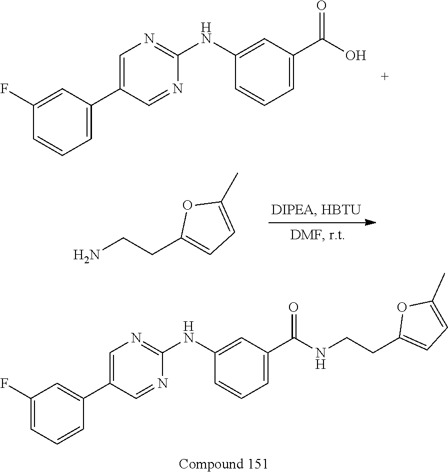

3-((5-(3-Fluorophenyl)pyrimidin-2-yl)amino)benzoic acid (100 mg, 0.32 mmol), 2-(5-methylfuran-2-yl)ethan-1-amine (45 mg, 0.36 mmol), and HBTU (184 mg, 0.48 mmol) were dissolved in DMF (3.2 mL), followed up by addition of DIPEA (0.08 mL, 0.48 mmol) and stirred for 18 h at room temperature. The reaction mixture was extracted by EA and brine. The organic layer was dried over anhydrous Na₂SO₄ and concentrated. The crude mixture was solidified by using EA to give compound 151, 3-((5-(3-fluorophenyl)pyrimidin-2-yl)amino)-N-(2-(5-methylfuran-2-yl)ethyl)benzamide (65 mg, 49%) as a beige solid.

Synthesis of Compound 152

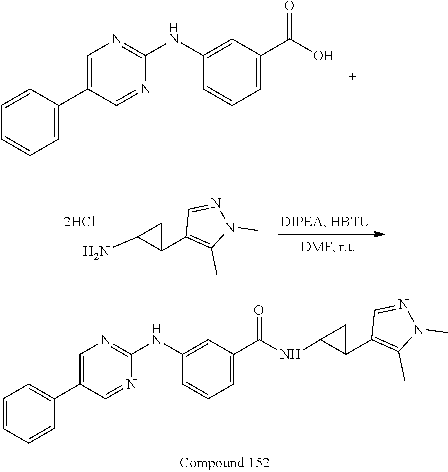 [SMILES: O=C(O)c1cccc(Nc2ncc(-c3ccccc3)cn2)c1 ; Cc1c(C2CC2N)cnn1C ; Cc1c(C2CC2NC(=O)c2cccc(Nc3ncc(-c4ccccc4)cn3)c2)cnn1C]

3-((5-Phenylpyrimidin-2-yl)amino)benzoic acid (100 mg, 0.34 mmol), 2-(1,5-dimethyl-1H-pyrazol-4-yl)cyclopropan-1-amine dihydrochloride (92 mg, 0.41 mmol), and HBTU (195 mg, 0.51 mmol) were dissolved in DMF (3.4 mL), followed up by addition of DIPEA (0.21 mL, 1.2 mmol) and stirred for 18 h at room temperature. The reaction mixture was extracted by EA and saturated aq. NaHCO₃. The organic layer was dried over anhydrous Na₂SO₄ and concentrated. The crude mixture was solidified by using EA to give compound 152, N-(2-(1,5-dimethyl-1H-pyrazol-4-yl)cyclopropyl)-3-((5-phenylpyrimidin-2-yl)amino)benzamide (107 mg, 73%) as a beige solid.

Synthesis of Compound 153

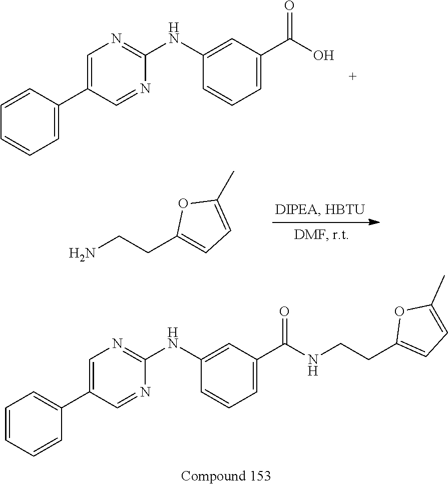

3-((5-Phenylpyrimidin-2-yl)amino)benzoic acid (100 mg, 0.34 mmol), 2-(5-methylfuran-2-yl)ethan-1-amine (52 mg, 0.41 mmol), and HBTU (195 mg, 0.51 mmol) were dissolved in DMF (3.4 mL), followed up by addition of DIPEA (0.09 mL, 0.51 mmol) and stirred for 18 h at room temperature. The reaction mixture was extracted by EA and brine. The organic layer was dried over anhydrous Na₂SO₄ and concentrated. The crude mixture was solidified by using EA to give compound 153, N-(2-(5-methylfuran-2-yl)ethyl)-3-((5-phenylpyrimidin-2-yl)amino)benzamide (87 mg, 64%) as a beige solid.

Synthesis of Compound 154

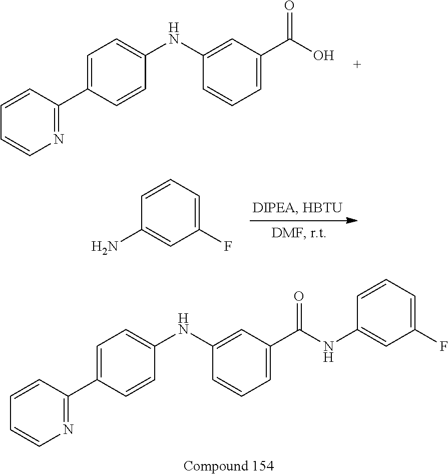 [SMILES: O=C(O)c1cccc(Nc2ccc(-c3ccccn3)cc2)c1 ; Nc1cccc(F)c1 ; O=C(Nc1cccc(F)c1)c1cccc(Nc2ccc(-c3ccccn3)cc2)c1]

3-((4-(Pyridin-2-yl)phenyl)amino)benzoic acid (50 mg, 0.17 mmol), 3-fluoroaniline (0.018 mL, 0.19 mmol), and HBTU (98 mg, 0.26 mmol) were dissolved in DMF (1.7 mL), followed up by addition of DIPEA (0.045 mL, 0.26 mmol) and stirred for 15.5 hours at room temperature. The reaction mixture was extracted by EA and brine. The organic layer was dried over anhydrous Na₂SO₄ and concentrated. The residue was purified by MPLC to give compound 154, N-(3-fluorophenyl)-3-((4-(pyridin-2-yl)phenyl)amino)benzamide (39 mg, 59%) as a white solid.

Synthesis of Compound 155

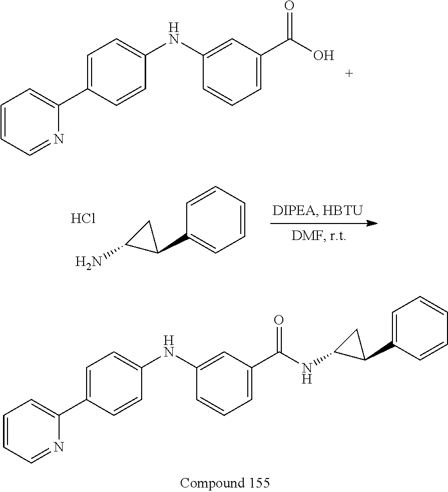 [SMILES: O=C(O)c1cccc(Nc2ccc(-c3ccccn3)cc2)c1 ; Cl ; N[C@@H]1C[C@H]1c1ccccc1 ; O=C(N[C@@H]1C[C@H]1c1ccccc1)c1cccc(Nc2ccc(-c3ccccn3)cc2)c1]

3-((4-(Pyridin-2-yl)phenyl)amino)benzoic acid (50 mg, 0.17 mmol), (1R,2S)-2-phenylcyclopropan-1-amine hydrochloride (32 mg, 0.19 mmol), and HBTU (98 mg, 0.26 mmol) were dissolved in DMF (1.7 mL), followed up by addition of DIPEA (0.045 mL, 0.26 mmol) and stirred for 15.5 hours at room temperature. The reaction mixture was extracted by EA and brine. The organic layer was dried over anhydrous Na₂SO₄ and concentrated. The residue was purified by MPLC to give compound 155, N-((1R,2S)-2-phenylcyclopropyl)-3-((4-(pyridin-2-yl)phenyl)amino)benzamide (66 mg, 95%) as a yellow solid.

Synthesis of Compound 156

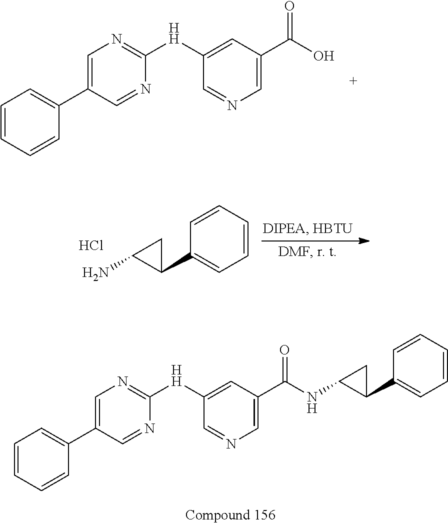

5-((5-Phenylpyrimidin-2-yl)amino)nicotinic acid (100 mg, 0.34 mmol), (1R,2S)-2-phenylcyclopropan-1-amine hydrochloride (64 mg, 0.38 mmol), and HBTU (194 mg, 0.51 mmol) were dissolved in DMF (3.4 mL), followed up by addition of DIPEA (0.179 mL, 1 mmol) and stirred for 18 hours at room temperature. The white solid was precipitated out of the solution, and the solution was filtered to give compound 156, N-((1R,2S)-2-phenylcyclopropyl)-5-((5-phenylpyrimidin-2-yl)amino)nicotinamide (111 mg, 80%) as a white solid.

Synthesis of Compound 157

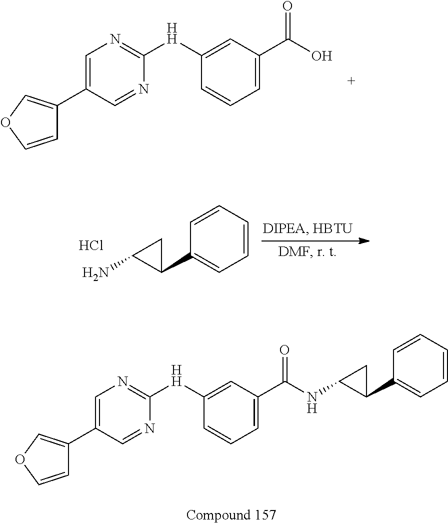

3-((5-(Furan-3-yl)pyrimidin-2-yl)amino)benzoic acid (60 mg, 0.21 mmol), (1R,2S)-2-phenylcyclopropan-1-amine hydrochloride (40 mg, 0.23 mmol), and HBTU (121 mg, 0.32 mmol) were dissolved in DMF (2.1 mL), followed up by addition of DIPEA (0.11 mL, 0.64 mmol) and stirred for 18 hours at room temperature. The reaction mixture was extracted by EA and brine. The organic layer was dried over anhydrous Na₂SO₄ and concentrated. The crude mixture was solidified by using EA to give compound 157, 3-((5-(furan-3-yl)pyrimidin-2-yl)amino)-N-((1R,2S)-2-phenylcyclopropyl)benzamide (60 mg, 71%) as a beige solid.

Synthesis of Compound 158

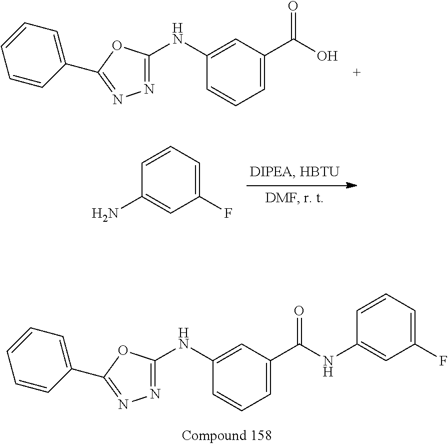 [SMILES: O=C(O)c1cccc(Nc2nnc(-c3ccccc3)o2)c1 ; Nc1cccc(F)c1 ; O=C(Nc1cccc(F)c1)c1cccc(Nc2nnc(-c3ccccc3)o2)c1]

3-((5-Phenyl-1,3,4-oxadiazol-2-yl)amino)benzoic acid (50 mg, 0.18 mmol), 3-fluoroaniline (0.019 mL, 0.20 mmol), and HBTU (101 mg, 0.27 mmol) were dissolved in DMF (1.8 mL), followed up by addition of DIPEA (0.046 mL, 0.27 mmol) and stirred for 24 hours at room temperature. The reaction mixture was extracted by EA and brine. The organic layer was dried over anhydrous MgSO₄ and concentrated. The residue was purified by MPLC and solidified by using acetone to give compound 158, N-(3-fluorophenyl)-3-((5-phenyl-1,3,4-oxadiazol-2-yl)amino)benzamide (24 mg, 36%) as a white solid.

Synthesis of Compound 159

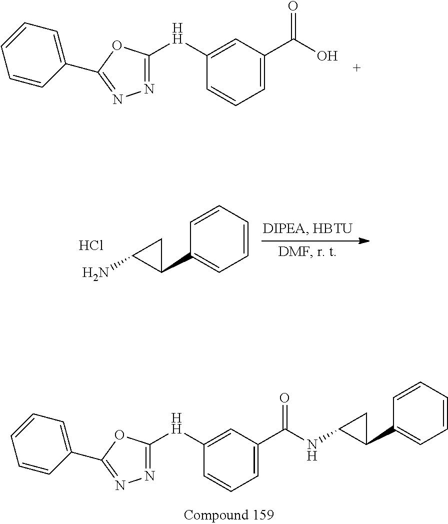

3-((5-Phenyl-1,3,4-oxadiazol-2-yl)amino)benzoic acid (50 mg, 0.18 mmol), (1R,2S)-2-phenylcyclopropan-1-amine hydrochloride (34 mg, 0.20 mmol), and HBTU (101 mg, 0.27 mmol) were dissolved in DMF (1.8 mL), followed up by addition of DIPEA (0.046 mL, 0.27 mmol) and stirred for 24 hours at room temperature. The reaction mixture was extracted by EA and brine. The organic layer was dried over anhydrous MgSO₄ and concentrated. The crude mixture was solidified by using acetone to give compound 159, 3-((5-phenyl-1,3,4-oxadiazol-2-yl)amino)-N-((1R,2S)-2-phenylcyclopropyl)benzamide (33 mg, 47%) as a yellow solid.

Synthesis of Compound 160

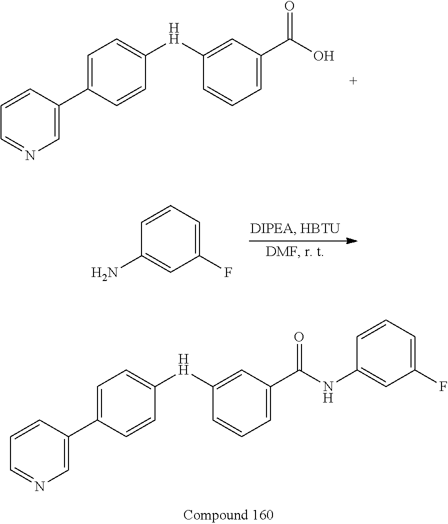

3-((4-(Pyridin-3-yl)phenyl)amino)benzoic acid (50 mg, 0.17 mmol), 3-fluoroaniline (0.018 mL, 0.19 mmol), and HBTU (98 mg, 0.26 mmol) were dissolved in DMF (1.7 mL), followed up by addition of DIPEA (0.045 mL, 0.26 mmol) and stirred for 24 hours at room temperature. The reaction mixture was extracted by EA and brine. The organic layer was dried over anhydrous MgSO₄ and concentrated. The residue was purified by MPLC to give compound 160, N-(3-fluorophenyl)-3-((4-(pyridin-3-yl)phenyl)amino)benzamide (21 mg, 30%) as a yellow solid.

Synthesis of Compound 161

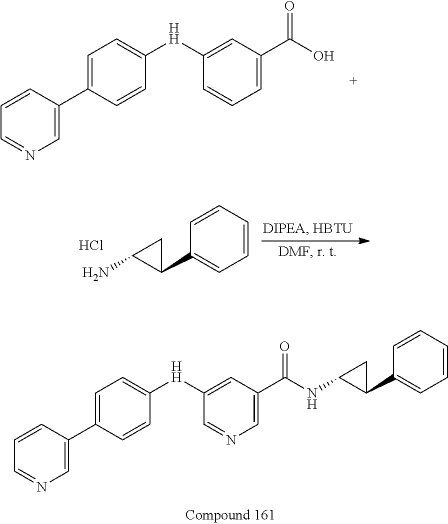

3-((4-(Pyridin-3-yl)phenyl)amino)benzoic acid (50 mg, 0.17 mmol), (1R,2S)-2-phenylcyclopropan-1-amine hydrochloride (32 mg, 0.19 mmol), and HBTU (98 mg, 0.26 mmol) were dissolved in DMF (1.7 mL), followed up by addition of DIPEA (0.045 mL, 0.26 mmol) and stirred for 24 hours at room temperature. The reaction mixture was extracted by EA and brine. The organic layer was dried over anhydrous MgSO₄ and concentrated. The residue was purified by MPLC to give compound 161, N-((1R,2S)-2-phenylcyclopropyl)-3-((4-(pyridin-3-yl)phenyl)amino)benzamide (33 mg, 47%) as a yellow solid.

Synthesis of Compound 162

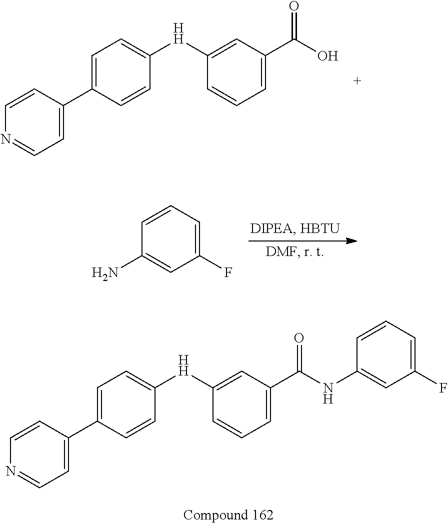

3-((4-(Pyridin-4-yl)phenyl)amino)benzoic acid (50 mg, 0.17 mmol), 3-fluoroaniline (0.018 mL, 0.19 mmol), and HBTU (98 mg, 0.26 mmol) were dissolved in DMF (1.7 mL), followed up by addition of DIPEA (0.045 mL, 0.26 mmol) and stirred for 24 hours at room temperature. The reaction mixture was extracted by EA and brine. The organic layer was dried over anhydrous MgSO₄ and concentrated. The residue was purified by MPLC to give compound 162, N-(3-fluorophenyl)-3-((4-(pyridin-4-yl)phenyl)amino)benzamide (19 mg, 29%) as a brown solid.

Synthesis of Compound 163

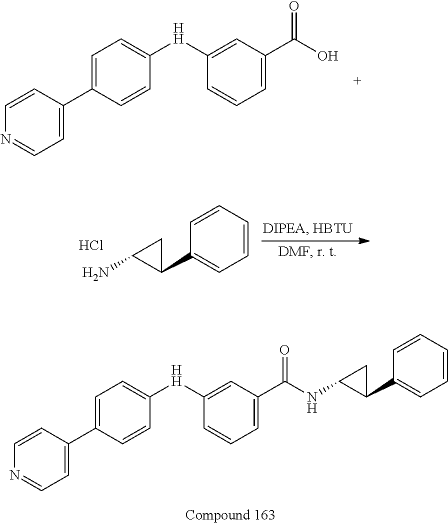

3-((4-(Pyridin-4-yl)phenyl)amino)benzoic acid (50 mg, 0.17 mmol), (1R,2S)-2-phenylcyclopropan-1-amine hydrochloride (32 mg, 0.19 mmol), and HBTU (98 mg, 0.26 mmol) were dissolved in DMF (1.7 mL), followed up by addition of DIPEA (0.045 mL, 0.26 mmol) and stirred for 24 hours at room temperature. The reaction mixture was extracted by EA and brine. The organic layer was dried over anhydrous MgSO₄ and concentrated. The residue was purified by MPLC to give compound 163, N-((1R,2S)-2-phenylcyclopropyl)-3-((4-(pyridin-4-yl)phenyl)amino)benzamide (26 mg, 37%) as a yellow solid.

Synthesis of Compound 164

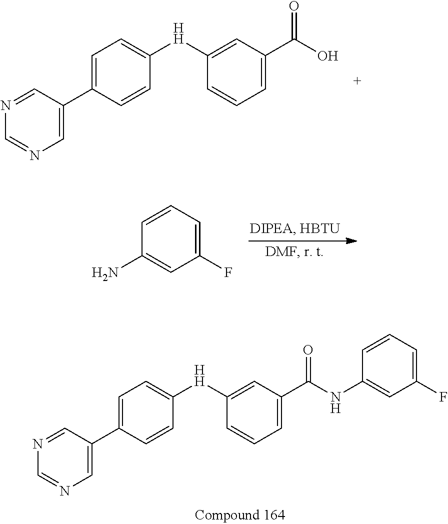

3-((4-(Pyrimidin-5-yl)phenyl)amino)benzoic acid (50 mg, 0.17 mmol), 3-fluoroaniline (0.018 mL, 0.19 mmol), and HBTU (98 mg, 0.26 mmol) were dissolved in DMF (1.7 mL), followed up by addition of DIPEA (0.045 mL, 0.26 mmol) and stirred for 18.5 hours at room temperature. The reaction mixture was extracted by EA and brine. The organic layer was dried over anhydrous Na₂SO₄ and concentrated. The crude mixture was solidified by using EA to give compound 164, N-(3-fluorophenyl)-3-((4-(pyrimidin-5-yl)phenyl)amino)benzamide (50 mg, 75%) as an ivory solid.

Synthesis of Compound 165

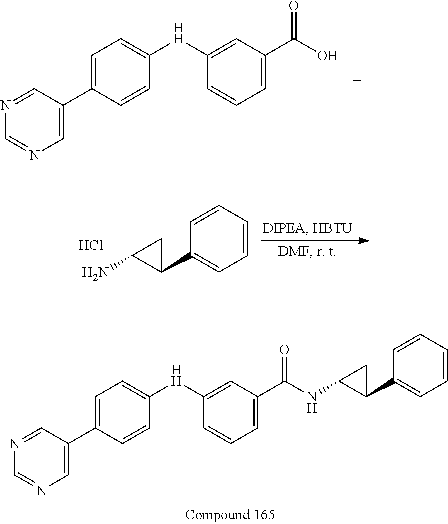

3-((4-(Pyrimidin-5-yl)phenyl)amino)benzoic acid (50 mg, 0.17 mmol), (1R,2S)-2-phenylcyclopropan-1-amine hydrochloride (32 mg, 0.19 mmol), and HBTU (98 mg, 0.26 mmol) were dissolved in DMF (1.7 mL), followed up by addition of DIPEA (0.045 mL, 0.26 mmol) and stirred for 18.5 hours at room temperature. The reaction mixture was extracted by EA and brine. The organic layer was dried over anhydrous Na₂SO₄ and concentrated. The crude mixture was solidified by using EA to give compound 165, N-((1R,2S)-2-phenylcyclopropyl)-3-((4-(pyrimidin-5-yl)phenyl)amino)benzamide (66 mg, 94%) as a bright pink solid.

Synthesis of Compound 166

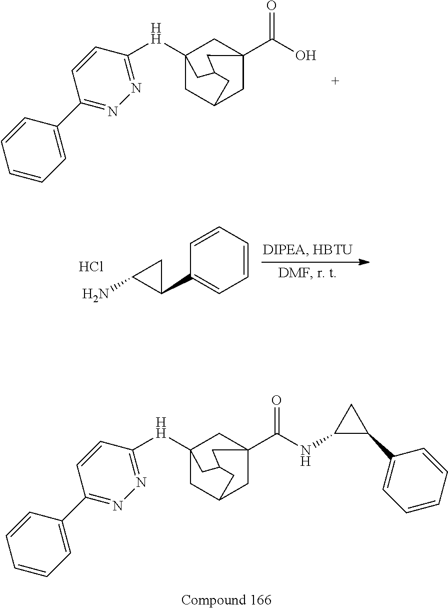

3-((6-Phenylpyridazin-3-yl)amino)adamantane-1-carboxylic acid (100 mg, 0.26 mmol), (1R,2S)-2-phenylcyclopropan-1-amine hydrochloride (53 mg, 0.31 mmol), and HBTU (163 mg, 0.43 mmol) were dissolved in DMF (2.9 mL), followed up by addition of DIPEA (0.149 mL, 0.86 mmol) and stirred for 18 hours at room temperature. The reaction mixture was extracted by EA and brine. The organic layer was dried over anhydrous Na₂SO₄ and concentrated. The residue was solidified by using EA and HEX to give compound 166, N-((1R,2S)-2-phenylcyclopropyl)-3-((6-phenylpyridazin-3-yl)amino)adamantane-1-carboxamide (70 mg, 53%) as a beige solid.

Synthesis of Compound 167

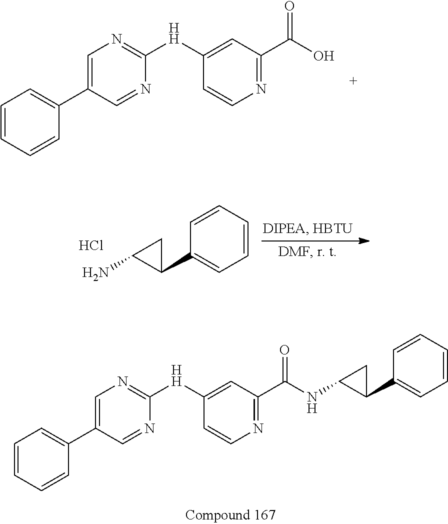

4-((5-Phenylpyrimidin-2-yl)amino)picolinic acid (100 mg, 0.35 mmol), (1R,2S)-2-phenylcyclopropan-1-amine hydrochloride (66 mg, 0.39 mmol), and HBTU (201 mg, 0.53 mmol) were dissolved in DMF (3.5 mL), followed up by addition of DIPEA (0.185 mL, 1.06 mmol) and stirred for 18 hours at room temperature. The white solid was precipitated out of the solution, and the solution was filtered to give compound 167, N-((1R,2S)-2-phenylcyclopropyl)-4-((5-phenylpyrimidin-2-yl)amino)picolinamide (97 mg, 67%) as a white solid.

Synthesis of Compound 168

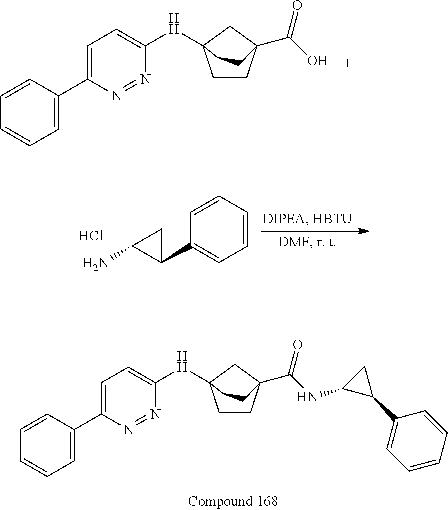

(1s,4s)-4-((6-Phenylpyridazin-3-yl)amino)bicyclo[2.2.1]heptane-1-carboxylic acid (100 mg, 0.32 mmol), (1R,2S)-2-phenylcyclopropan-1-amine hydrochloride (60 mg, 0.36 mmol), and HBTU (184 mg, 0.48 mmol) were dissolved in DMF (3.2 mL), followed up by addition of DIPEA (0.169 mL, 0.97 mmol) and stirred for 18 hours at room temperature. The reaction mixture was extracted by EA and brine. The organic layer was dried over anhydrous Na₂SO₄ and concentrated. The residue was solidified by using EA and HEX to give compound 168, (1s,4s)-N-((1R,2S)-2-phenylcyclopropyl)-4-((6-phenylpyridazin-3-yl)amino)bicyclo[2.2.1]heptane-1-carboxamide (91 mg, 66%) as a beige solid.

Synthesis of Compound 170

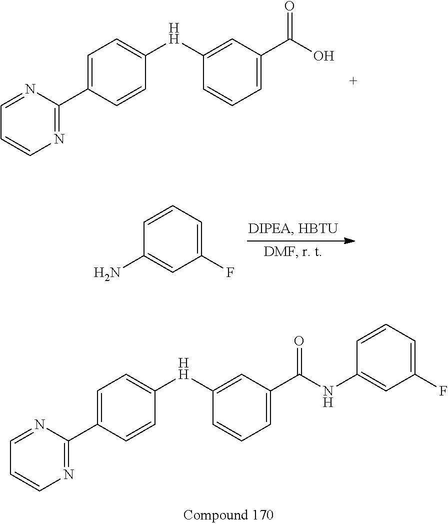

3-((4-(Pyrimidin-2-yl)phenyl)amino)benzoic acid (146 mg, 0.5 mmol), 3-fluoroaniline (0.053 mL, 0.55 mmol), and HBTU (284 mg, 0.75 mmol) were dissolved in DMF (5 mL), followed up by addition of DIPEA (0.13 mL, 0.75 mmol) and stirred for 24 hours at room temperature. The reaction mixture was extracted by EA and brine. The organic layer was dried over anhydrous MgSO₄ and concentrated. The residue was purified by MPLC to give compound 170, N-(3-fluorophenyl)-3-((4-(pyrimidin-2-yl)phenyl)amino)benzamide (111 mg, 57%) as a pale yellow solid.

Synthesis of Compound 171

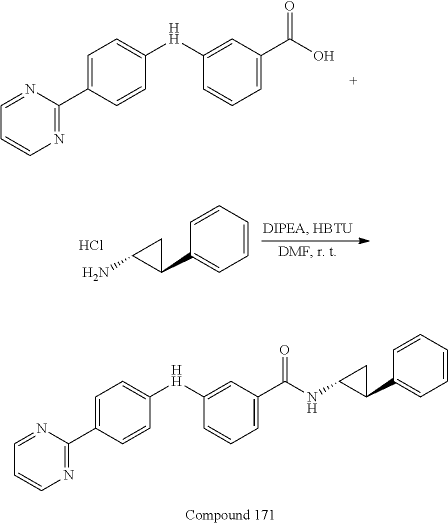

3-((4-(Pyrimidin-2-yl)phenyl)amino)benzoic acid (146 mg, 0.5 mmol), (1R,2S)-2-phenylcyclopropan-1-amine hydrochloride (94 mg, 0.55 mmol), and HBTU (284 mg, 0.75 mmol) were dissolved in DMF (5 mL), followed up by addition of DIPEA (0.13 mL, 0.75 mmol) and stirred for 24 hours at room temperature. The reaction mixture was extracted by EA and brine. The organic layer was dried over anhydrous MgSO₄ and concentrated. The residue was purified by MPLC to give compound 171, N-((1R,2S)-2-phenylcyclopropyl)-3-((4-(pyrimidin-2-yl)phenyl)amino)benzamide (98 mg, 48%) as a white solid.

Synthesis of Compound 172

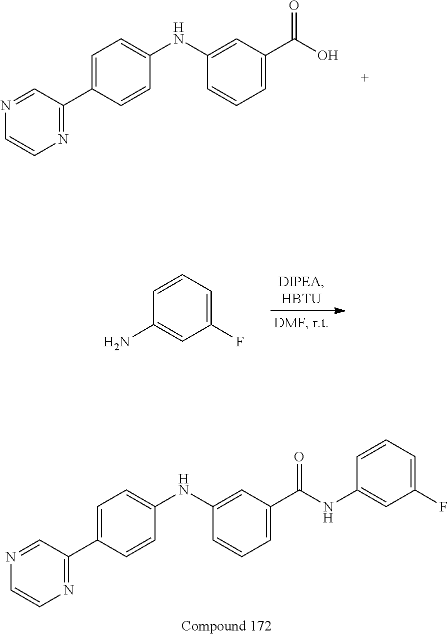 [SMILES: O=C(O)c1cccc(Nc2ccc(-c3cnccn3)cc2)c1 ; Nc1cccc(F)c1 ; O=C(Nc1cccc(F)c1)c1cccc(Nc2ccc(-c3cnccn3)cc2)c1]

3-((4-(Pyrazin-2-yl)phenyl)amino)benzoic acid (146 mg, 0.5 mmol), 3-fluoroaniline (0.053 mL, 0.55 mmol), and HBTU (284 mg, 0.75 mmol) were dissolved in DMF (5 mL), followed up by addition of DIPEA (0.13 mL, 0.75 mmol) and stirred for 24 hours at room temperature. The reaction mixture was extracted by EA and brine. The organic layer was dried over anhydrous MgSO₄ and concentrated. The residue was purified by MPLC to give compound 172, N-(3-fluorophenyl)-3-((4-(pyrazin-2-yl)phenyl)amino)benzamide (29 mg, 15%) as a pale yellow solid.

Synthesis of Compound 173

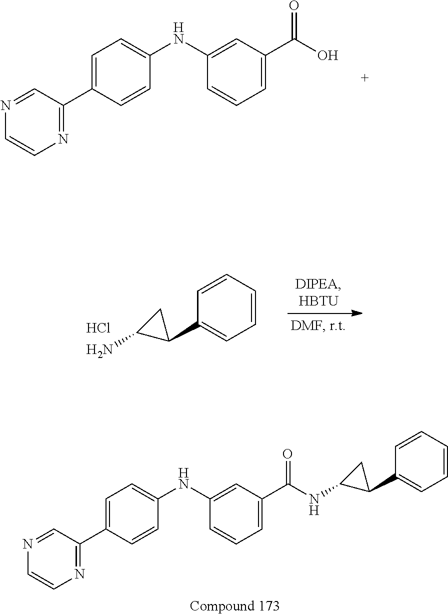 [SMILES: O=C(O)c1cccc(Nc2ccc(-c3cnccn3)cc2)c1 ; Cl ; N[C@@H]1C[C@H]1c1ccccc1 ; O=C(N[C@@H]1C[C@H]1c1ccccc1)c1cccc(Nc2ccc(-c3cnccn3)cc2)c1]

3-((4-(Pyrazin-2-yl)phenyl)amino)benzoic acid (146 mg, 0.5 mmol), (1R,2S)-2-phenylcyclopropan-1-amine hydrochloride (94 mg, 0.55 mmol), and HBTU (284 mg, 0.75 mmol) were dissolved in DMF (5 mL), followed up by addition of DIPEA (0.13 mL, 0.75 mmol) and stirred for 24 hours at room temperature. The reaction mixture was extracted by EA and brine. The organic layer was dried over anhydrous MgSO₄ and concentrated. The residue was purified by MPLC to give compound 173, N-((1R,2S)-2-phenylcyclopropyl)-3-((4-(pyrazin-2-yl)phenyl)amino)benzamide (66 mg, 32%) as a white solid.

Synthesis of Compound 174

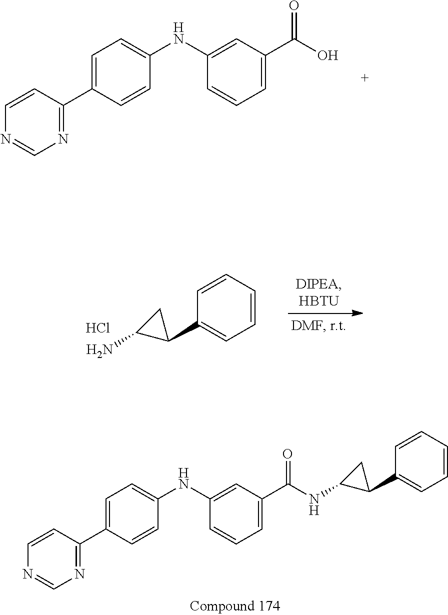 [SMILES: O=C(O)c1cccc(Nc2ccc(-c3ccncn3)cc2)c1 ; Cl ; N[C@@H]1C[C@H]1c1ccccc1 ; O=C(N[C@@H]1C[C@H]1c1ccccc1)c1cccc(Nc2ccc(-c3ccncn3)cc2)c1]

3-((4-(Pyrimidin-4-yl)phenyl)amino)benzoic acid (64 mg, 0.22 mmol), (1R,2S)-2-phenylcyclopropan-1-amine hydrochloride (41 mg, 0.24 mmol), and HBTU (125 mg, 0.33 mmol) were dissolved in DMF (2.2 mL), followed up by addition of DIPEA (0.057 mL, 0.33 mmol) and stirred for 24 hours at room temperature. The reaction mixture was extracted by EA and brine. The organic layer was dried over anhydrous MgSO₄ and concentrated. The residue was purified by MPLC to give compound 174, N-((1R,2S)-2-phenylcyclopropyl)-3-((4-(pyrimidin-4-yl)phenyl)amino)benzamide (44 mg, 50%) as a white solid.

Synthesis of Compound 175

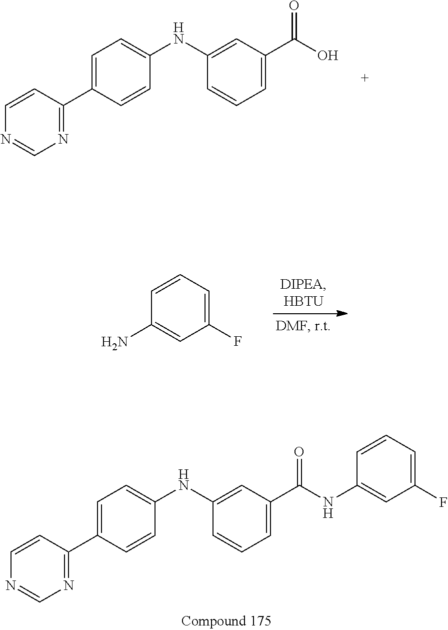 [SMILES: O=C(O)c1cccc(Nc2ccc(-c3ccncn3)cc2)c1 ; Nc1cccc(F)c1 ; O=C(Nc1cccc(F)c1)c1cccc(Nc2ccc(-c3ccncn3)cc2)c1]

3-((4-(Pyrimidin-4-yl)phenyl)amino)benzoic acid (64 mg, 0.22 mmol), 3-fluoroaniline (0.023 mL, 0.24 mmol), and HBTU (125 mg, 0.33 mmol) were dissolved in DMF (2.2 mL), followed up by addition of DIPEA (0.057 mL, 0.33 mmol) and stirred for 24 hours at room temperature. The reaction mixture was extracted by EA and brine. The organic layer was dried over anhydrous MgSO₄ and concentrated. The residue was purified by MPLC to give compound 175, N-(3-fluorophenyl)-3-((4-(pyrimidin-4-yl)phenyl)amino)benzamide (23 mg, 27%) as an orange solid.

Synthesis of Compound 176

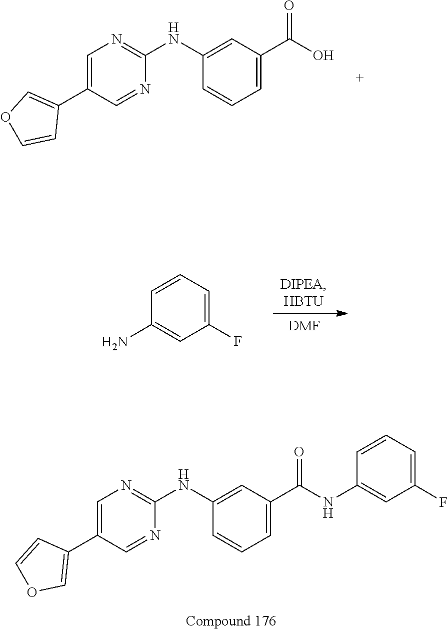

3-((5-(Furan-3-yl)pyrimidin-2-yl)amino)benzoic acid (100 mg, 0.36 mmol), 3-fluoroaniline (44 mg, 0.39 mmol), and HBTU (202 mg, 0.53 mmol) were dissolved in DMF (3.6 mL), followed up by addition of DIPEA (0.093 mL, 0.53 mmol) and stirred for overnight at room temperature and stirred for overnight at 50° C. and stirred for overnight at 70° C. The reaction mixture was extracted by EA and brine. The organic layer was dried over anhydrous Na₂SO₄ and concentrated. The crude mixture was solidified by using EA to give compound 176, N-(3-fluorophenyl)-3-((5-(furan-3-yl)pyrimidin-2-yl)amino)benzamide (34 mg, 26%) as an orange brown solid.

Synthesis of Compound 177

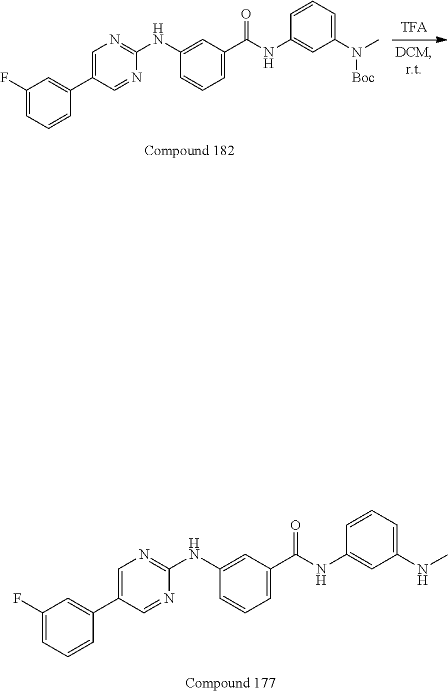 [SMILES: CN(C(=O)OC(C)(C)C)c1cccc(NC(=O)c2cccc(Nc3ncc(-c4cccc(F)c4)cn3)c2)c1 ; CNc1cccc(NC(=O)c2cccc(Nc3ncc(-c4cccc(F)c4)cn3)c2)c1]

tert-Butyl (3-(3-((5-(3-fluorophenyl)pyrimidin-2-yl)amino)benzamido)phenyl)(methyl) carbamate (90 mg, 0.18 mmol) was dissolved in DCM (1.8 mL), followed up by addition of TFA (0.26 mL) and stirred for 1 hour at room temperature. The reaction mixture was extracted by DCM and aq. NaOH (1 M). The organic layer was dried over anhydrous Na₂SO₄ and concentrated. The reaction mixture was solidified by using EA to give compound 177, 3-((5-(3-fluorophenyl)pyrimidin-2-yl)amino)-N-(3-(methylamino)phenyl)benzamide (52 mg, 72%) as a white solid.

Synthesis of Compound 178

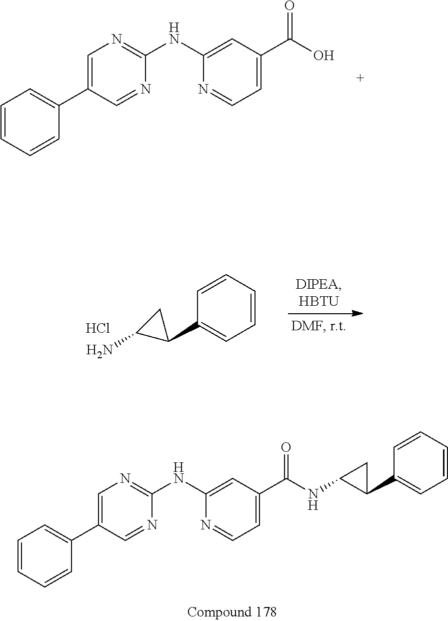 [SMILES: O=C(O)c1ccnc(Nc2ncc(-c3ccccc3)cn2)c1 ; Cl ; N[C@@H]1C[C@H]1c1ccccc1 ; O=C(N[C@@H]1C[C@H]1c1ccccc1)c1ccnc(Nc2ncc(-c3ccccc3)cn2)c1]

2-((5-Phenylpyrimidin-2-yl)amino)isonicotinic acid (100 mg, 0.34 mmol), (1R,2S)-2-phenylcyclopropan-1-amine hydrochloride (64 mg, 0.38 mmol), and HBTU (195 mg, 0.51 mmol) were dissolved in DMF (3.4 mL), followed up by addition of DIPEA (0.179 mL, 1.03 mmol) and stirred for overnight at room temperature. The white solid was precipitated out of the solution, and the solution was filtered and washed with EA to give compound 178, N-((1R,2S)-2-phenylcyclopropyl)-2-((5-phenylpyrimidin-2-yl)amino)isonicotinamide (113 mg, 81%) as a white solid.

Synthesis of Compound 179

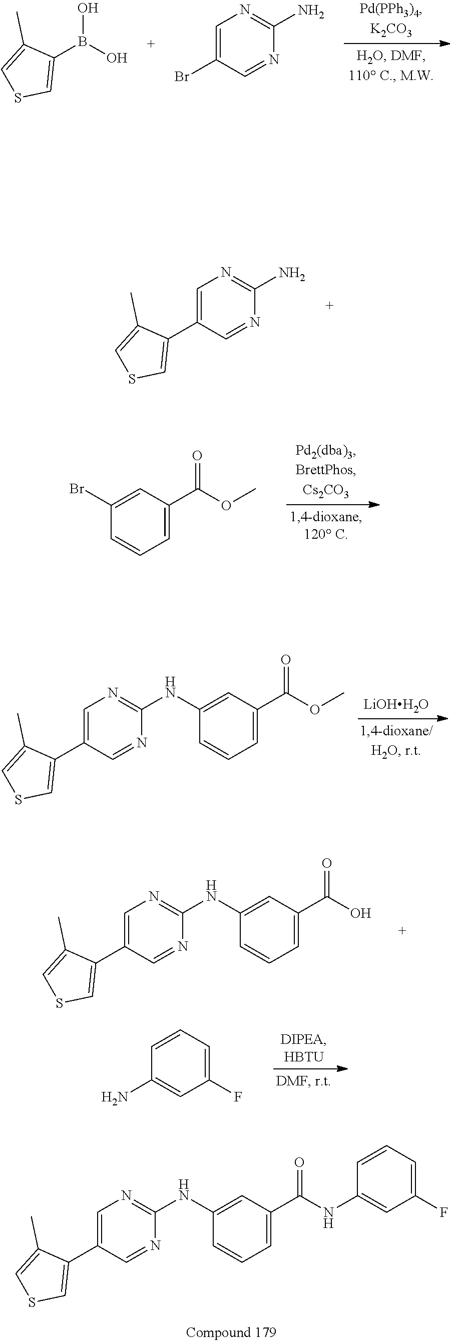

Step 1: (4-Methylthiophen-3-yl)boronic acid (902 mg, 6.35 mmol), 5-bromopyrimidin-2-amine (850 mg, 4.88 mmol), Pd(PPh₃)₄ (282 mg, 0.244 mmol), and potassium carbonate (2.03 g, 14.65 mmol) were mixed in H₂O/DMF (10/10 mL) and heated in a microwave reactor for 35 minutes at 110° C. The reaction mixture was extracted by EA and brine. The organic layer was dried over anhydrous Na₂SO₄ and concentrated. The residue was solidified by using EA and HEX to give 5-(4-methylthiophen-3-yl)pyrimidin-2-amine (496 mg, 53%) as a beige solid.

Step 2: 5-(4-Methylthiophen-3-yl)pyrimidin-2-amine (490 mg, 2.6 mmol), methyl 3-bromobenzoate (661 mg, 3.07 mmol), Pd₂(dba)₃ (235 mg, 0.26 mmol), BrettPhos (275 mg, 0.51 mmol), and cesium carbonate (1.67 g, 5.12 mmol) were mixed in 1,4-dioxane (13 mL) and heated in a microwave reactor for 90 minutes at 120° C. The reaction mixture was extracted by EA and brine. The organic layer was dried over anhydrous Na₂SO₄ and concentrated. The residue was solidified by using EA and HEX to give methyl 3-((5-(4-methylthiophen-3-yl)pyrimidin-2-yl)amino)benzoate (364 mg, 44%) as a white solid.

Step 3: Methyl 3-((5-(4-methylthiophen-3-yl)pyrimidin-2-yl)amino)benzoate (350 mg, 1.08 mmol) and LiOH.H₂O (451 mg, 10.76 mmol) were mixed in H₂O/1,4-dioxane (4.5/22 mL) and stirred for overnight at room temperature. Then pH value of the solution was adjusted to 3 by 1 N HCl. The reaction mixture was extracted by EA. The organic layer was dried over anhydrous Na₂SO₄ and concentrated. The residue was solidified by using EA and HEX to give 3-((5-(4-methylthiophen-3-yl)pyrimidin-2-yl)amino)benzoic acid (311 mg, 93%) as a white solid.

Step 4: 3-((5-(4-Methylthiophen-3-yl)pyrimidin-2-yl)amino)benzoic acid (100 mg, 0.32 mmol), 3-fluoroaniline (0.039 mg, 0.35 mmol), and HBTU (183 mg, 0.48 mmol) were dissolved in DMF (3.2 mL), followed up by addition of DIPEA (0.084 mL, 0.48 mmol) and stirred for overnight at 60° C., and stirred for overnight at 70° C. The reaction mixture was extracted by EA and brine. The organic layer was dried over anhydrous Na₂SO₄ and concentrated. The crude mixture was solidified by using EA and HEX to give compound 179, N-(3-fluorophenyl)-3-((5-(4-methylthiophen-3-yl)pyrimidin-2-yl)amino)benzamide (46 mg, 36%) as a beige solid.

Synthesis of Compound 181

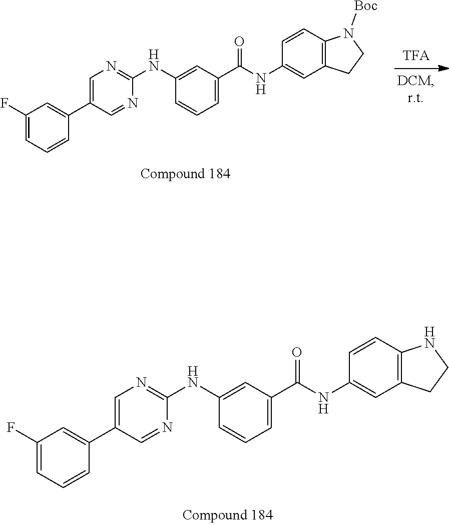 [SMILES: CC(C)(C)OC(=O)N1CCc2cc(NC(=O)c3cccc(Nc4ncc(-c5cccc(F)c5)cn4)c3)ccc21 ; O=C(Nc1ccc2c(c1)CCN2)c1cccc(Nc2ncc(-c3cccc(F)c3)cn2)c1]

tert-Butyl 5-(3-((5-(3-fluorophenyl)pyrimidin-2-yl)amino)benzamido)indoline-1-carboxylate (55 mg, 0.1 mmol) was dissolved in DCM (1 mL), followed up by addition of TFA (0.16 mL) and stirred for 1 hour at room temperature. The reaction mixture was extracted by DCM and saturated aq. NaOH (1 M). The organic layer was dried over anhydrous Na₂SO₄ and concentrated. The reaction mixture was solidified by using EA and HEX to give compound 181, 3-((5-(3-fluorophenyl)pyrimidin-2-yl)amino)-N-(indolin-5-yl)benzamide (26 mg, 59%) as a grey solid.

Synthesis of Compound 182

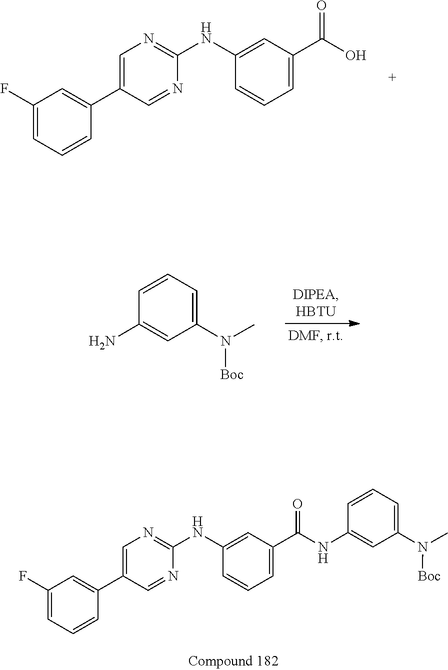 [SMILES: O=C(O)c1cccc(Nc2ncc(-c3cccc(F)c3)cn2)c1 ; CN(C(=O)OC(C)(C)C)c1cccc(N)c1 ; CN(C(=O)OC(C)(C)C)c1cccc(NC(=O)c2cccc(Nc3ncc(-c4cccc(F)c4)cn3)c2)c1]

3-((5-(3-Fluorophenyl)pyrimidin-2-yl)amino)benzoic acid (100 mg, 0.32 mmol), tert-butyl (3-aminophenyl)(methyl)carbamate (79 mg, 0.36 mmol), and HBTU (184 mg, 0.48 mmol) were dissolved in DMF (3.2 mL), followed up by addition of DIPEA (0.85 mL, 0.48 mmol) and stirred for overnight at room temperature. The reaction mixture was extracted by EA and brine. The organic layer was dried over anhydrous Na₂SO₄ and concentrated. The crude mixture was solidified by using EA and HEX to give compound 182, tert-butyl (3-(3-((5-(3-fluorophenyl)pyrimidin-2-yl)amino)benzamido)phenyl)(methyl)carbamate (109 mg, 66%) as a beige solid.

Synthesis of Compound 183

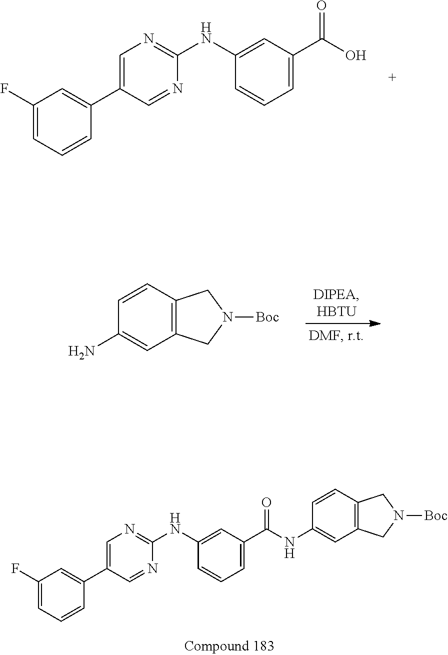 [SMILES: O=C(O)c1cccc(Nc2ncc(-c3cccc(F)c3)cn2)c1 ; CC(C)(C)OC(=O)N1Cc2ccc(N)cc2C1 ; CC(C)(C)OC(=O)N1Cc2ccc(NC(=O)c3cccc(Nc4ncc(-c5cccc(F)c5)cn4)c3)cc2C1]

3-((5-(3-Fluorophenyl)pyrimidin-2-yl)amino)benzoic acid (100 mg, 0.32 mmol), tert-butyl 5-aminoisoindoline-2-carboxylate (83 mg, 0.36 mmol), and HBTU (184 mg, 0.48 mmol) were dissolved in DMF (3.2 mL), followed up by addition of DIPEA (0.085 mL, 0.48 mmol) and stirred for overnight at room temperature. The reaction mixture was extracted by EA and brine. The organic layer was dried over anhydrous Na₂SO₄ and concentrated. The crude mixture was solidified by using EA and HEX to give compound 183, tert-butyl 5-(3-((5-(3-fluorophenyl)pyrimidin-2-yl)amino)benzamido)isoindoline-2-carboxylate (59 mg, 35%) as a white solid.

Synthesis of Compound 184

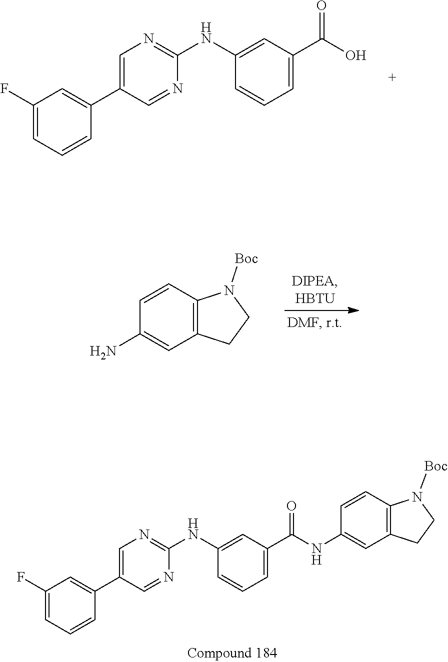 [SMILES: O=C(O)c1cccc(Nc2ncc(-c3cccc(F)c3)cn2)c1 ; CC(C)(C)OC(=O)N1CCc2cc(N)ccc21 ; CC(C)(C)OC(=O)N1CCc2cc(NC(=O)c3cccc(Nc4ncc(-c5cccc(F)c5)cn4)c3)ccc21]

3-((5-(3-Fluorophenyl)pyrimidin-2-yl)amino)benzoic acid (100 mg, 0.32 mmol), tert-butyl 5-aminoindoline-1-carboxylate (83 mg, 0.36 mmol), and HBTU (184 mg, 0.48 mmol) were dissolved in DMF (3.2 mL), followed up by addition of DIPEA (0.085 mL, 0.48 mmol) and stirred for overnight at room temperature. The reaction mixture was extracted by EA and brine. The organic layer was dried over anhydrous Na₂SO₄ and concentrated. The crude mixture was solidified by using EA to give compound 184, tert-butyl 5-(3-((5-(3-fluorophenyl)pyrimidin-2-yl)amino)benzamido)indoline-1-carboxylate (117 mg, 69%) as a grey solid.

Synthesis of Compound 192

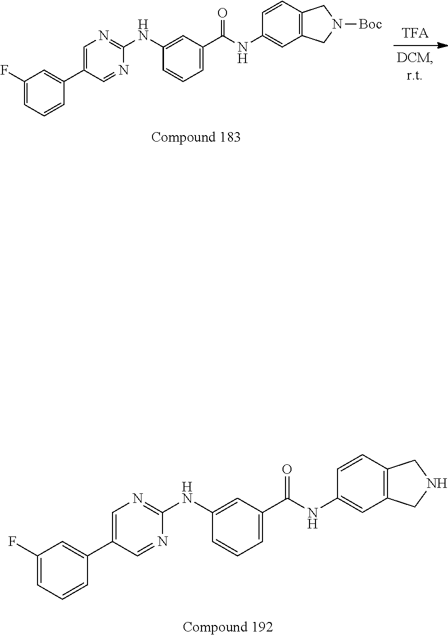 [SMILES: CC(C)(C)OC(=O)N1Cc2ccc(NC(=O)c3cccc(Nc4ncc(-c5cccc(F)c5)cn4)c3)cc2C1 ; O=C(Nc1ccc2c(c1)CNC2)c1cccc(Nc2ncc(-c3cccc(F)c3)cn2)c1]

tert-Butyl 5-(3-((5-(3-fluorophenyl)pyrimidin-2-yl)amino)benzamido)isoindoline-2-carboxylate (55 mg, 0.1 mmol) was dissolved in DCM (1 mL), followed up by addition of TFA (0.16 mL) and stirred for 1 hour at room temperature. The reaction mixture was extracted by DCM and saturated aq. NaOH (1 M). The organic layer was dried over anhydrous Na₂SO₄ and concentrated. The reaction mixture was solidified by using EA and HEX, and slurry with MeOH, and filtrate was concentrated to give compound 192, 3-((5-(3-fluorophenyl)pyrimidin-2-yl)amino)-N-(isoindolin-5-yl)benzamide (7 mg, 15%) as a white solid.

Synthesis of Compound 193

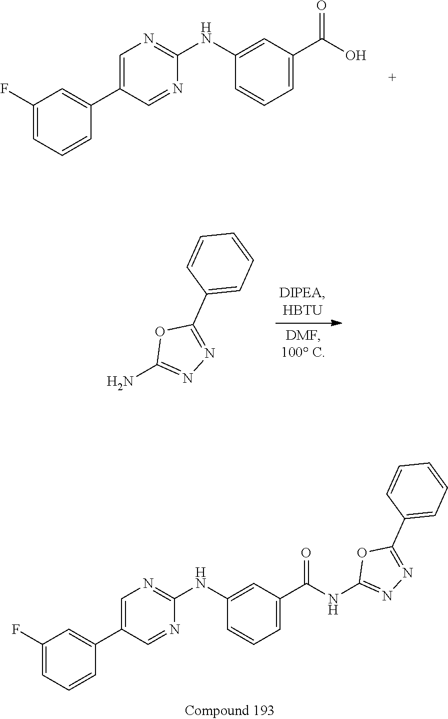 [SMILES: O=C(O)c1cccc(Nc2ncc(-c3cccc(F)c3)cn2)c1 ; Nc1nnc(-c2ccccc2)o1 ; O=C(Nc1nnc(-c2ccccc2)o1)c1cccc(Nc2ncc(-c3cccc(F)c3)cn2)c1]

3-((5-(3-Fluorophenyl)pyrimidin-2-yl)amino)benzoic acid (100 mg, 0.32 mmol), 5-phenyl-1,3,4-oxadiazol-2-amine (57 mg, 0.36 mmol), and HBTU (184 mg, 0.48 mmol) were dissolved in DMF (3.2 mL), followed up by addition of DIPEA (0.084 mL, 0.48 mmol) and stirred for overnight at 100° C. The reaction mixture was extracted by EA and brine. The organic layer was dried over anhydrous Na₂SO₄ and concentrated. The residue was purified by MPLC to give compound 193, 3-((5-(3-fluorophenyl)pyrimidin-2-yl)amino)-N-(5-phenyl-1,3,4-oxadiazol-2-yl)benzamide (15 mg, 10%) as a white solid.

Synthesis of Compound 194

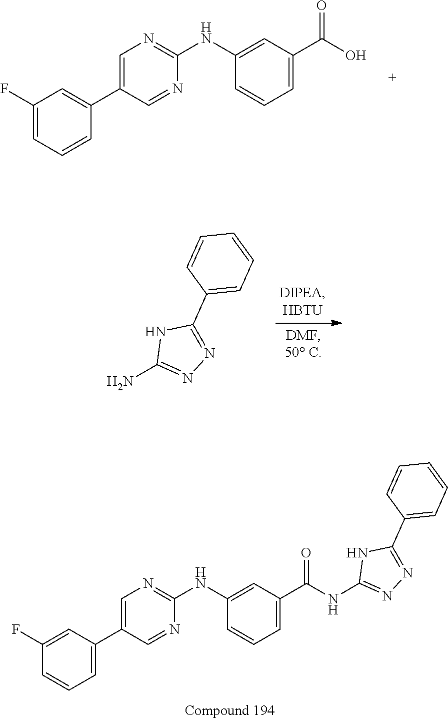 [SMILES: O=C(O)c1cccc(Nc2ncc(-c3cccc(F)c3)cn2)c1 ; Nc1nnc(-c2ccccc2)[nH]1 ; O=C(Nc1nnc(-c2ccccc2)[nH]1)c1cccc(Nc2ncc(-c3cccc(F)c3)cn2)c1]

3-((5-(3-Fluorophenyl)pyrimidin-2-yl)amino)benzoic acid (100 mg, 0.32 mmol), 5-phenyl-4H-1,2,4-triazol-3-amine (57 mg, 0.36 mmol), and HBTU (184 mg, 0.48 mmol) were dissolved in DMF (3.2 mL), followed up by addition of DIPEA (0.084 mL, 0.48 mmol) and stirred for overnight at 50° C. The white solid was precipitated out of the solution, and the solution was filtered, and washed with EA to give compound 194, 3-((5-(3-fluorophenyl)pyrimidin-2-yl)amino)-N-(5-phenyl-4H-1,2,4-triazol-3-yl)benzamide (45 mg, 31%) as a white solid.

Synthesis of Compound 195

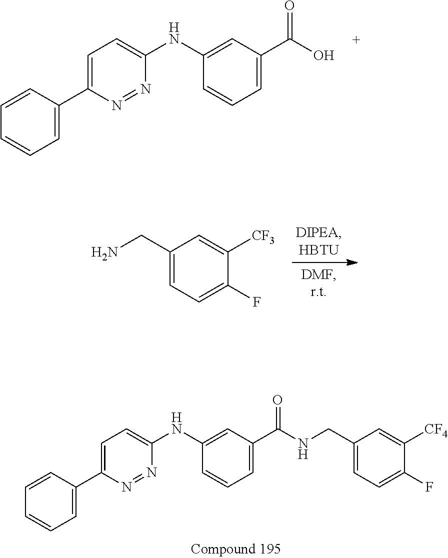

3-((6-Phenylpyridazin-3-yl)amino)benzoic acid (30 mg, 0.10 mmol), (4-fluoro-3-(trifluoromethyl)phenyl)methanamine (0.02 mL, 0.12 mmol), and HBTU (59 mg, 0.16 mmol) were dissolved in DMF (1 mL), followed up by addition of DIPEA (0.05 mL, 0.31 mmol) and stirred for 5 hours at room temperature. The reaction mixture was extracted by EA and brine. The organic layer was dried over anhydrous Na₂SO₄ and concentrated. The residue was purified by MPLC. The crude mixture was solidified by using EA and HEX to give compound 195, N-(4-fluoro-3-(trifluoromethyl)benzyl)-3-((6-phenylpyridazin-3-yl)amino)benzamide (23 mg, 49%) as a white solid.

Synthesis of Compound 196

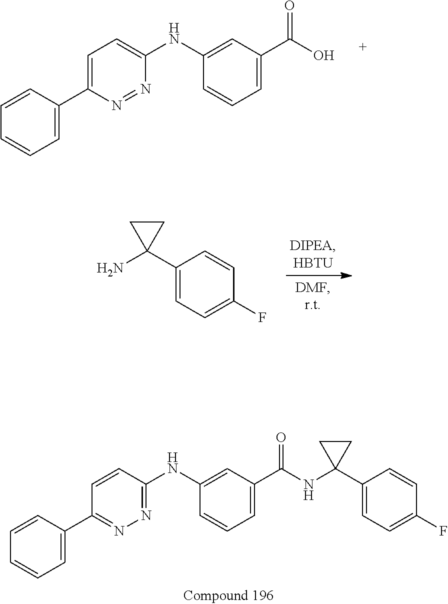 [SMILES: O=C(O)c1cccc(Nc2ccc(-c3ccccc3)nn2)c1 ; NC1(c2ccc(F)cc2)CC1 ; O=C(NC1(c2ccc(F)cc2)CC1)c1cccc(Nc2ccc(-c3ccccc3)nn2)c1]

3-((6-Phenylpyridazin-3-yl)amino)benzoic acid (30 mg, 0.10 mmol), 1-(4-fluorophenyl)cyclopropan-1-amine (0.02 mL, 0.12 mmol), and HBTU (59 mg, 0.16 mmol) were dissolved in DMF (1 mL), followed up by addition of DIPEA (0.05 mL, 0.31 mmol) and stirred for 5 hours at room temperature. The reaction mixture was extracted by EA and brine. The organic layer was dried over anhydrous Na₂SO₄ and concentrated. The residue was purified by MPLC. The crude mixture was solidified by using EA, ether, and HEX to give compound 196, N-(1-(4-fluorophenyl)cyclopropyl)-3-((6-phenylpyridazin-3-yl)amino)benzamide (9 mg, 19%) as a white solid.

Synthesis of Compound 197

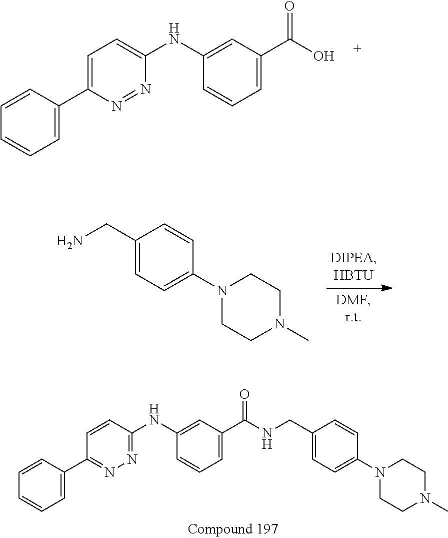 [SMILES: O=C(O)c1cccc(Nc2ccc(-c3ccccc3)nn2)c1 ; CN1CCN(c2ccc(CN)cc2)CC1 ; CN1CCN(c2ccc(CNC(=O)c3cccc(Nc4ccc(-c5ccccc5)nn4)c3)cc2)CC1]

3-((6-Phenylpyridazin-3-yl)amino)benzoic acid (60 mg, 0.21 mmol), (4-(4-methylpiperazin-1-yl)phenyl)methanamine (51 mg, 0.25 mmol), and HBTU (117 mg, 0.31 mmol) were dissolved in DMF (2 mL), followed up by addition of DIPEA (0.11 mL, 0.62 mmol) and stirred for 18 hours at room temperature. The reaction mixture was extracted by EA and brine. The organic layer was dried over anhydrous Na₂SO₄ and concentrated. The residue was purified by MPLC. The crude mixture was solidified by using EA and HEX to give compound 197, N-(4-(4-methylpiperazin-1-yl)benzyl)-3-((6-phenylpyridazin-3-yl)amino)benzamide (80 mg, 81%) as a white solid.

Synthesis of Compound 198

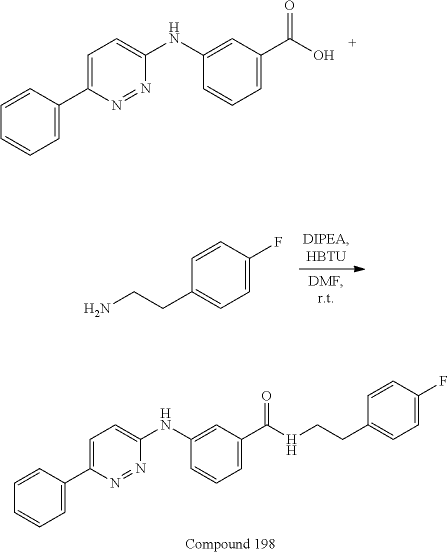

3-((6-Phenylpyridazin-3-yl)amino)benzoic acid (30 mg, 0.10 mmol), 2-(4-fluorophenyl)ethan-1-amine (0.02 mL, 0.12 mmol), and HBTU (59 mg, 0.16 mmol) were dissolved in DMF (1 mL), followed up by addition of DIPEA (0.05 mL, 0.31 mmol) and stirred for 5 hours at room temperature. The reaction mixture was extracted by EA and brine. The organic layer was dried over anhydrous Na₂SO₄ and concentrated. The residue was purified by MPLC. The crude mixture was solidified by using EA and HEX to give compound 198, N-(4-fluorophenethyl)-3-((6-phenylpyridazin-3-yl)amino)benzamide (12 mg, 27%) as a white solid.

Synthesis of Compound 199

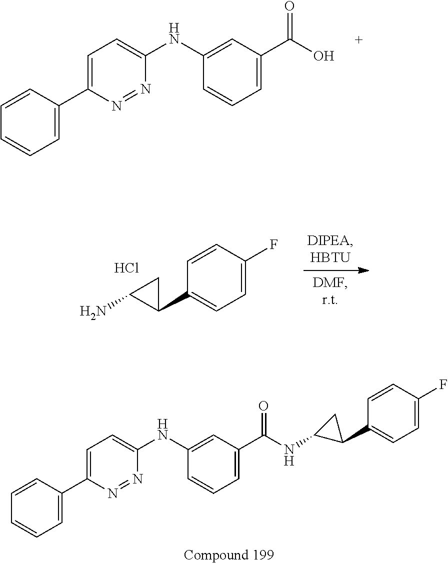 [SMILES: O=C(O)c1cccc(Nc2ccc(-c3ccccc3)nn2)c1 ; N[C@@H]1C[C@H]1c1ccc(F)cc1 ; Cl ; O=C(N[C@@H]1C[C@H]1c1ccc(F)cc1)c1cccc(Nc2ccc(-c3ccccc3)nn2)c1]

3-((6-Phenylpyridazin-3-yl)amino)benzoic acid (30 mg, 0.10 mmol), (1R,2S)-2-phenylcyclopropan-1-amine hydrochloride (23 mg, 0.12 mmol), and HBTU (59 mg, 0.16 mmol) were dissolved in DMF (1 mL), followed up by addition of DIPEA (0.05 mL, 0.31 mmol) and stirred for 5 hours at room temperature. The reaction mixture was extracted by EA and brine. The organic layer was dried over anhydrous Na₂SO₄ and concentrated. The residue was purified by MPLC. The crude mixture was solidified by using EA and HEX to give compound 199, N-((1R,2S)-2-phenylcyclopropyl)-3-((6-phenylpyridazin-3-yl)amino)benzamide (32 mg, 72%) as a white solid.

Synthesis of Compound 200

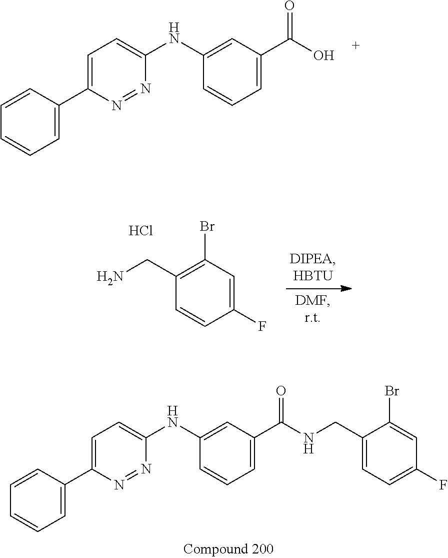 [SMILES: O=C(O)c1cccc(Nc2ccc(-c3ccccc3)nn2)c1 ; NCc1ccc(F)cc1Br ; Cl ; O=C(NCc1ccc(F)cc1Br)c1cccc(Nc2ccc(-c3ccccc3)nn2)c1]

3-((6-Phenylpyridazin-3-yl)amino)benzoic acid (100 mg, 0.34 mmol), (2-bromo-4-fluorophenyl)methanamine hydrochloride (99 mg, 0.41 mmol), and HBTU (195 mg, 0.52 mmol) were dissolved in DMF (4 mL), followed up by addition of DIPEA (0.30 mL, 1.72 mmol) and stirred for 20 hours at room temperature. The reaction mixture was extracted by EA and brine. The organic layer was dried over anhydrous Na₂SO₄ and concentrated. The residue was purified by MPLC. The crude mixture was solidified by using EA and HEX to give compound 200, N-(2-bromo-4-fluorobenzyl)-3-((6-phenylpyridazin-3-yl)amino)benzamide (126 mg, 77%) as a white solid.

Synthesis of Compound 201

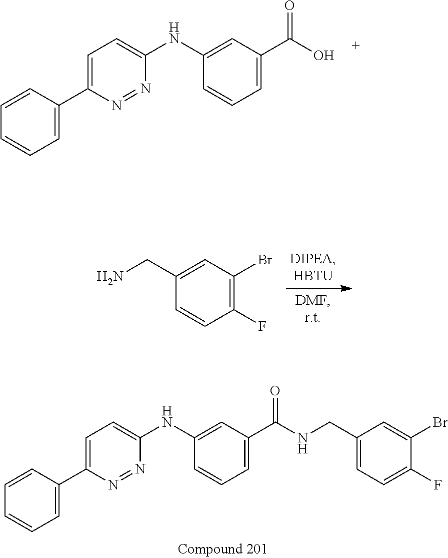 [SMILES: O=C(O)c1cccc(Nc2ccc(-c3ccccc3)nn2)c1 ; NCc1ccc(F)c(Br)c1 ; O=C(NCc1ccc(F)c(Br)c1)c1cccc(Nc2ccc(-c3ccccc3)nn2)c1]

3-((6-Phenylpyridazin-3-yl)amino)benzoic acid (100 mg, 0.34 mmol), (3-bromo-4-fluorophenyl)methanamine (84 mg, 0.41 mmol), and HBTU (195 mg, 0.52 mmol) were dissolved in DMF (4 mL), followed up by addition of DIPEA (0.18 mL, 1.03 mmol) and stirred for 20 hours at room temperature. The reaction mixture was extracted by EA and brine. The organic layer was dried over anhydrous Na₂SO₄ and concentrated. The residue was purified by MPLC. The crude mixture was solidified by using EA and HEX to give compound 201, N-(3-bromo-4-fluorobenzyl)-3-((6-phenylpyridazin-3-yl)amino)benzamide (145 mg, 88%) as a white solid.

Synthesis of Compound 202

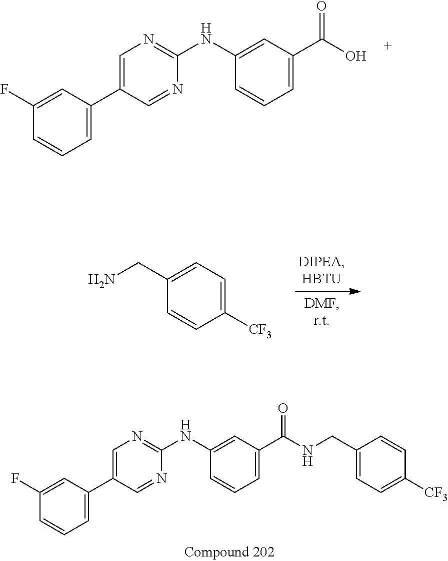 [SMILES: O=C(O)c1cccc(Nc2ncc(-c3cccc(F)c3)cn2)c1 ; NCc1ccc(C(F)(F)F)cc1 ; O=C(NCc1ccc(C(F)(F)F)cc1)c1cccc(Nc2ncc(-c3cccc(F)c3)cn2)c1]

3-((5-(3-Fluorophenyl)pyrimidin-2-yl)amino)benzoic acid (50 mg, 0.16 mmol), (4-(trifluoromethyl)phenyl)methanamine (34 mg, 0.19 mmol), and HBTU (92 mg, 0.24 mmol) were dissolved in DMF (2 mL), followed up by addition of DIPEA (0.04 mL, 0.24 mmol) and stirred for 20 hours at room temperature. The reaction mixture was concentrated under reduced pressure and the residue was dissolved in MeOH and sonicated. The slurry was filtered off and washed with MeOH to give compound 202, 3-((5-(3-fluorophenyl)pyrimidin-2-yl)amino)-N-(4-(trifluoromethyl)benzyl)benzamide (64 mg, 86%) as a white solid.

Synthesis of Compound 203

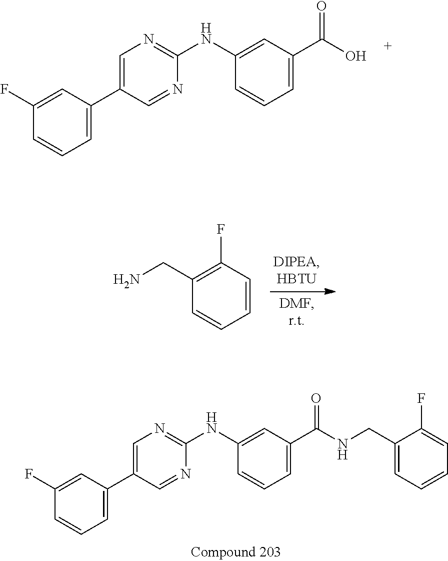 [SMILES: O=C(O)c1cccc(Nc2ncc(-c3cccc(F)c3)cn2)c1 ; NCc1ccccc1F ; O=C(NCc1ccccc1F)c1cccc(Nc2ncc(-c3cccc(F)c3)cn2)c1]

3-((5-(3-Fluorophenyl)pyrimidin-2-yl)amino)benzoic acid (50 mg, 0.16 mmol), (2-fluorophenyl)methanamine (0.02 mL, 0.19 mmol), and HBTU (92 mg, 0.24 mmol) were dissolved in DMF (2 mL), followed up by addition of DIPEA (0.04 mL, 0.24 mmol) and stirred for 20 hours at room temperature. The reaction mixture was concentrated under reduced pressure and the residue was dissolved in MeOH and sonicated. The slurry was filtered off and washed with MeOH to give compound 203, N-(2-fluorobenzyl)-3-((5-(3-fluorophenyl)pyrimidin-2-yl)amino)benzamide (57 mg, 86%) as a white solid.

Synthesis of Compound 204

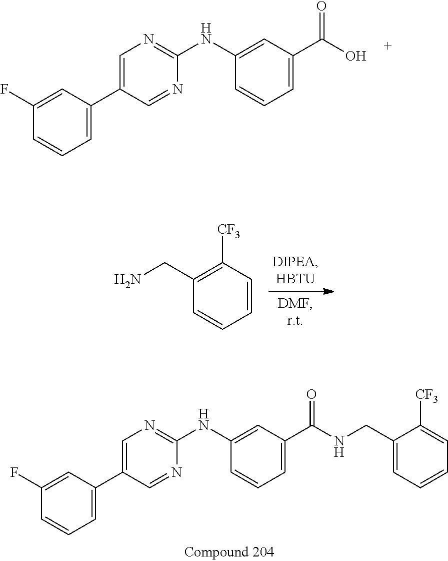 [SMILES: O=C(O)c1cccc(Nc2ncc(-c3cccc(F)c3)cn2)c1 ; NCc1ccccc1C(F)(F)F ; O=C(NCc1ccccc1C(F)(F)F)c1cccc(Nc2ncc(-c3cccc(F)c3)cn2)c1]

3-((5-(3-Fluorophenyl)pyrimidin-2-yl)amino)benzoic acid (50 mg, 0.16 mmol), (2-(trifluoromethyl)phenyl)methanamine (0.03 mL, 0.19 mmol), and HBTU (92 mg, 0.24 mmol) were dissolved in DMF (2 mL), followed up by addition of DIPEA (0.04 mL, 0.24 mmol) and stirred for 20 hours at room temperature. The reaction mixture was concentrated under reduced pressure and the residue was dissolved in MeOH and sonicated. The slurry was filtered off and washed with MeOH to give compound 204, 3-((5-(3-fluorophenyl)pyrimidin-2-yl)amino)-N-(2-(trifluoromethyl)benzyl)benzamide (53 mg, 71%) as a white solid.

Synthesis of Compound 205

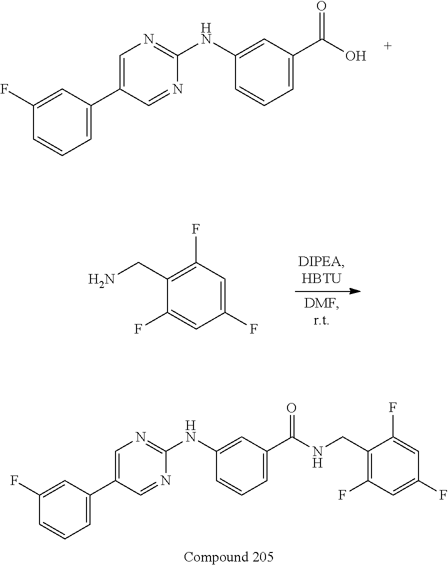 [SMILES: O=C(O)c1cccc(Nc2ncc(-c3cccc(F)c3)cn2)c1 ; NCc1c(F)cc(F)cc1F ; O=C(NCc1c(F)cc(F)cc1F)c1cccc(Nc2ncc(-c3cccc(F)c3)cn2)c1]

3-((5-(3-Fluorophenyl)pyrimidin-2-yl)amino)benzoic acid (50 mg, 0.16 mmol), (2,4,6-trifluorophenyl)methanamine (0.02 mL, 0.19 mmol), and HBTU (92 mg, 0.24 mmol) were dissolved in DMF (2 mL), followed up by addition of DIPEA (0.04 mL, 0.24 mmol) and stirred for 20 hours at room temperature. The reaction mixture was concentrated under reduced pressure and the residue was dissolved in MeOH and sonicated. The slurry was filtered off and washed with MeOH to give compound 205, 3-((5-(3-fluorophenyl)pyrimidin-2-yl)amino)-N-(2,4,6-trifluorobenzyl)benzamide (61 mg, 85%) as a white solid.

Synthesis of Compound 206

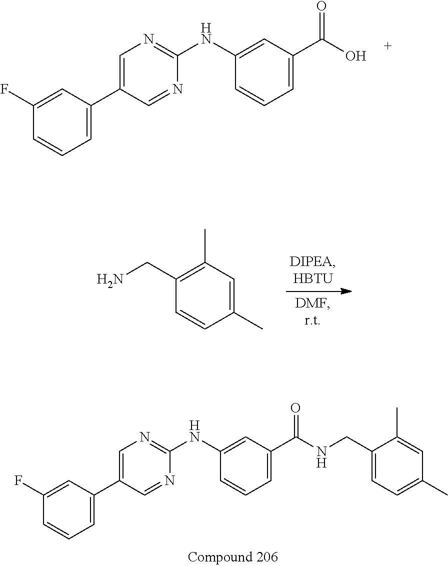

3-((5-(3-Fluorophenyl)pyrimidin-2-yl)amino)benzoic acid (50 mg, 0.16 mmol), (2,4-dimethylphenyl)methanamine (0.03 mL, 0.19 mmol), and HBTU (92 mg, 0.24 mmol) were dissolved in DMF (2 mL), followed up by addition of DIPEA (0.04 mL, 0.24 mmol) and stirred for 20 hours at room temperature The reaction mixture was concentrated under reduced pressure and the residue was dissolved in MeOH and sonicated. The slurry was filtered off and washed with MeOH to give compound 206, N-(2,4-dimethylbenzyl)-3-((5-(3-fluorophenyl)pyrimidin-2-yl)amino)benzamide (62 mg, 90%) as a white solid.

Synthesis of Compound 207

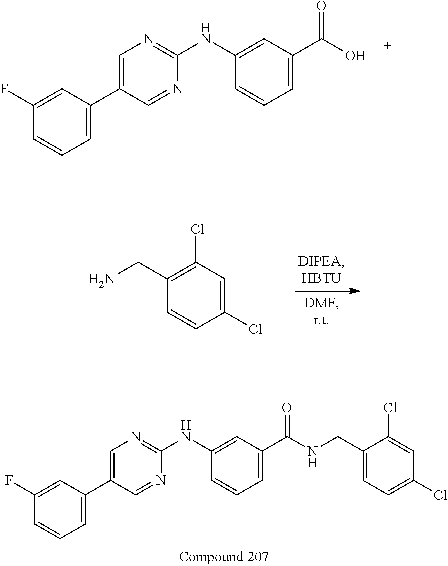 [SMILES: O=C(O)c1cccc(Nc2ncc(-c3cccc(F)c3)cn2)c1 ; NCc1ccc(Cl)cc1Cl ; O=C(NCc1ccc(Cl)cc1Cl)c1cccc(Nc2ncc(-c3cccc(F)c3)cn2)c1]

3-((5-(3-Fluorophenyl)pyrimidin-2-yl)amino)benzoic acid (50 mg, 0.16 mmol), (2,4-dichlorophenyl)methanamine (0.03 mL, 0.19 mmol), and HBTU (92 mg, 0.24 mmol) were dissolved in DMF (2 mL), followed up by addition of DIPEA (0.04 mL, 0.24 mmol) and stirred for 20 hours at room temperature. The reaction mixture was concentrated under reduced pressure and the residue was dissolved in MeOH and sonicated. The slurry was filtered off and washed with MeOH to give compound 207, N-(2,4-dichlorobenzyl)-3-((5-(3-fluorophenyl)pyrimidin-2-yl)amino)benzamide (57 mg, 76%) as a white solid.

Synthesis of Compound 208

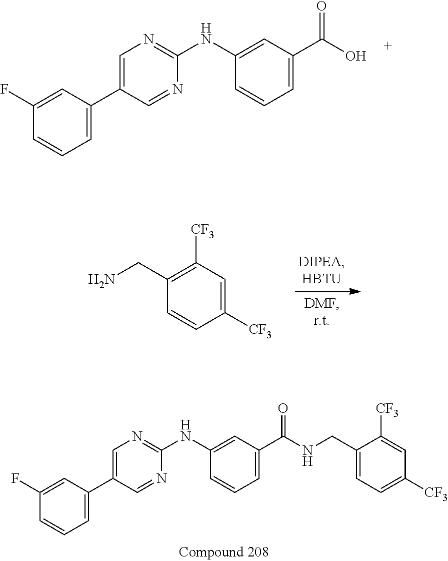 [SMILES: O=C(O)c1cccc(Nc2ncc(-c3cccc(F)c3)cn2)c1 ; NCc1ccc(C(F)(F)F)cc1C(F)(F)F ; O=C(NCc1ccc(C(F)(F)F)cc1C(F)(F)F)c1cccc(Nc2ncc(-c3cccc(F)c3)cn2)c1]

3-((5-(3-Fluorophenyl)pyrimidin-2-yl)amino)benzoic acid (50 mg, 0.16 mmol), (2,4-bis(trifluoromethyl)phenyl)methanamine (47 mg, 0.19 mmol), and HBTU (92 mg, 0.24 mmol) were dissolved in DMF (2 mL), followed up by addition of DIPEA (0.04 mL, 0.24 mmol) and stirred for 20 hours at room temperature. The reaction mixture was concentrated under reduced pressure and the residue was dissolved in MeOH and sonicated. The slurry was filtered off and washed with MeOH to give compound 208, N-(2,4-bis(trifluoromethyl)benzyl)-3-((5-(3-fluorophenyl)pyrimidin-2-yl)amino)benzamide (37 mg, 43%) as a white solid.

Synthesis of Compound 209

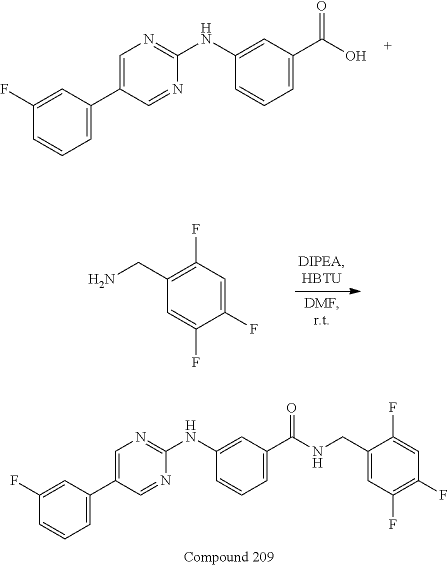 [SMILES: O=C(O)c1cccc(Nc2ncc(-c3cccc(F)c3)cn2)c1 ; NCc1cc(F)c(F)cc1F ; O=C(NCc1cc(F)c(F)cc1F)c1cccc(Nc2ncc(-c3cccc(F)c3)cn2)c1]

3-((5-(3-Fluorophenyl)pyrimidin-2-yl)amino)benzoic acid (50 mg, 0.16 mmol), (2,4,5-trifluorophenyl)methanamine (31 mg, 0.19 mmol), and HBTU (92 mg, 0.24 mmol) were dissolved in DMF (2 mL), followed up by addition of DIPEA (0.04 mL, 0.24 mmol) and stirred for 20 hours at room temperature. The reaction mixture was concentrated under reduced pressure and the residue was dissolved in MeOH and sonicated. The slurry was filtered off and washed with MeOH to give compound 209, 3-((5-(3-fluorophenyl)pyrimidin-2-yl)amino)-N-(2,4,5-trifluorobenzyl)benzamide (51 mg, 70%) as a white solid.

Synthesis of Compound 210

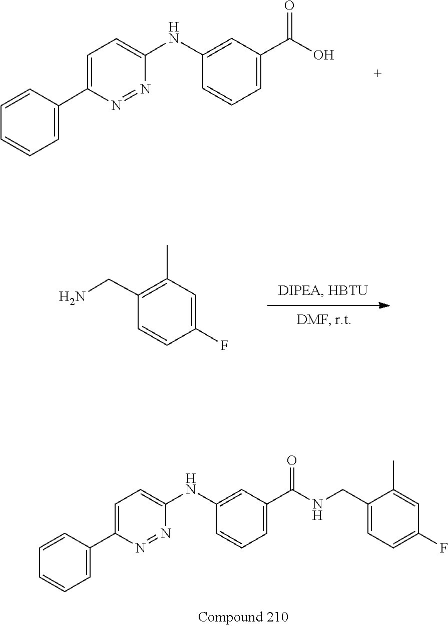

3-((6-Phenylpyridazin-3-yl)amino)benzoic acid (30 mg, 0.10 mmol), (4-fluoro-2-methylphenyl)methanamine (0.02 mL, 0.12 mmol), and HBTU (59 mg, 0.16 mmol) were dissolved in DMF (1 mL), followed up by addition of DIPEA (0.05 mL, 0.31 mmol) and stirred for 5 hours at room temperature. The reaction mixture was extracted by EA and brine. The organic layer was dried over anhydrous Na₂SO₄ and concentrated. The residue was purified by MPLC. The crude mixture was solidified by using EA and HEX to give compound 210, N-(4-fluoro-2-methylbenzyl)-3-((6-phenylpyridazin-3-yl)amino)benzamide (31 mg, 72%) as a white solid.

Synthesis of Compound 211

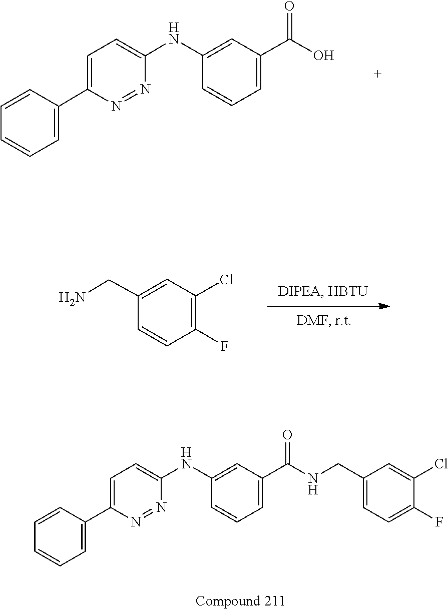 [SMILES: O=C(O)c1cccc(Nc2ccc(-c3ccccc3)nn2)c1 ; NCc1ccc(F)c(Cl)c1 ; O=C(NCc1ccc(F)c(Cl)c1)c1cccc(Nc2ccc(-c3ccccc3)nn2)c1]

3-((6-phenylpyridazin-3-yl)amino)benzoic acid (30 mg, 0.10 mmol), (3-chloro-4-fluorophenyl)methanamine (0.02 mL, 0.12 mmol), and HBTU (59 mg, 0.16 mmol) were dissolved in DMF (1 mL), followed up by addition of DIPEA (0.05 mL, 0.31 mmol) and stirred for 5 hours at room temperature. The reaction mixture was extracted by EA and brine. The organic layer was dried over anhydrous Na₂SO₄ and concentrated. The residue was purified by MPLC. The crude mixture was solidified by using EA and HEX to give compound 211, N-(3-chloro-4-fluorobenzyl)-3-((6-phenylpyridazin-3-yl)amino)benzamide (20 mg, 44%) as a white solid.

Synthesis of Compound 212

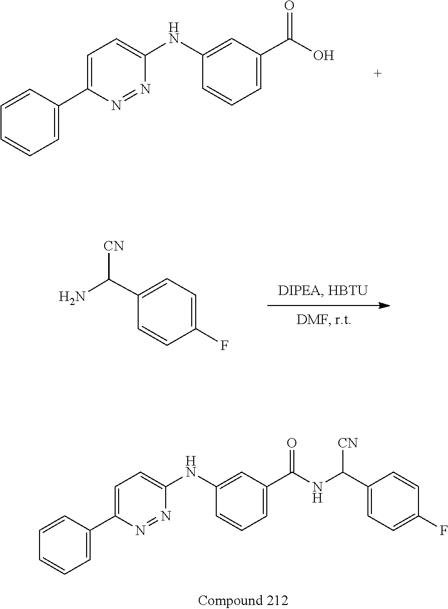 [SMILES: O=C(O)c1cccc(Nc2ccc(-c3ccccc3)nn2)c1 ; N#CC(N)c1ccc(F)cc1 ; N#CC(NC(=O)c1cccc(Nc2ccc(-c3ccccc3)nn2)c1)c1ccc(F)cc1]

3-((6-Phenylpyridazin-3-yl)amino)benzoic acid (30 mg, 0.10 mmol), 2-amino-2-(4-fluorophenyl)acetonitrile (19 mg, 0.12 mmol), and HBTU (59 mg, 0.16 mmol) were dissolved in DMF (1 mL), followed up by addition of DIPEA (0.05 mL, 0.31 mmol) and stirred for 19 hours at room temperature. The reaction mixture was extracted by EA and brine. The organic layer was dried over anhydrous Na₂SO₄ and concentrated. The residue was purified by MPLC. The crude mixture was solidified by using EA and HEX to give compound 212, N-(cyano(4-fluorophenyl)methyl)-3-((6-phenylpyridazin-3-yl)amino)benzamide (33 mg, 76%) as a white solid.

Synthesis of Compound 213

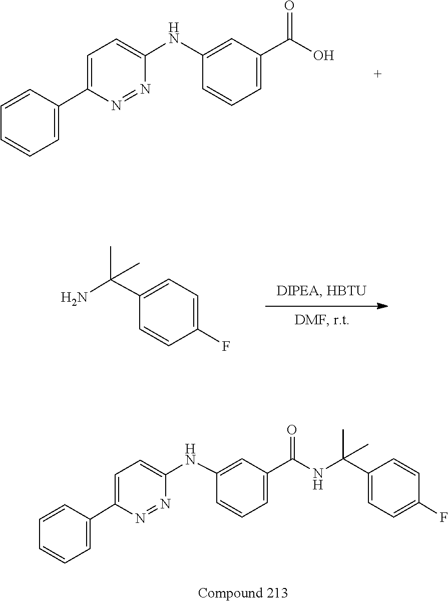 [SMILES: O=C(O)c1cccc(Nc2ccc(-c3ccccc3)nn2)c1 ; CC(C)(N)c1ccc(F)cc1 ; CC(C)(NC(=O)c1cccc(Nc2ccc(-c3ccccc3)nn2)c1)c1ccc(F)cc1]

3-((6-Phenylpyridazin-3-yl)amino)benzoic acid (30 mg, 0.10 mmol), 2-(4-fluorophenyl)propan-2-amine (0.02 mL, 0.12 mmol), and HBTU (59 mg, 0.16 mmol) were dissolved in DMF (1 mL), followed up by addition of DIPEA (0.05 mL, 0.31 mmol) and stirred for 19 hours at room temperature. The reaction mixture was extracted by EA and brine. The organic layer was dried over anhydrous Na₂SO₄ and concentrated. The residue was purified by MPLC. The crude mixture was solidified by using EA and HEX to give compound 213, N-(2-(4-fluorophenyl)propan-2-yl)-3-((6-phenylpyridazin-3-yl)amino)benzamide (36 mg, 81%) as a white solid.

Synthesis of Compound 214

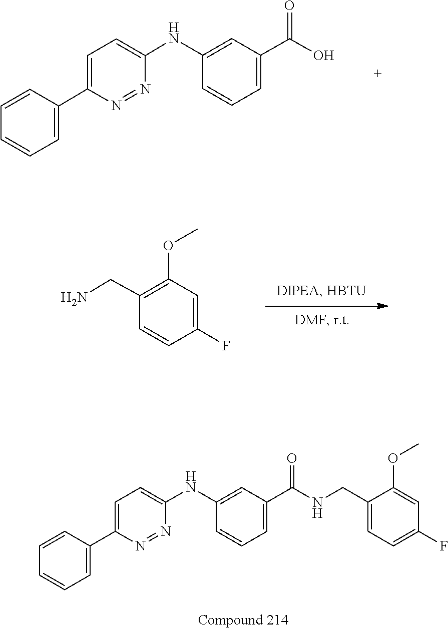 [SMILES: O=C(O)c1cccc(Nc2ccc(-c3ccccc3)nn2)c1 ; COc1cc(F)ccc1CN ; COc1cc(F)ccc1CNC(=O)c1cccc(Nc2ccc(-c3ccccc3)nn2)c1]

3-((6-Phenylpyridazin-3-yl)amino)benzoic acid (30 mg, 0.10 mmol), (4-fluoro-2-methoxyphenyl)methanamine (0.02 mL, 0.12 mmol), and HBTU (59 mg, 0.16 mmol) were dissolved in DMF (1 mL), followed up by addition of DIPEA (0.05 mL, 0.31 mmol) and stirred for 19 hours at room temperature. The reaction mixture was extracted by EA and brine. The organic layer was dried over anhydrous Na₂SO₄ and concentrated. The residue was purified by MPLC. The crude mixture was solidified by using EA and HEX to give compound 214, N-(4-fluoro-2-methoxybenzyl)-3-((6-phenylpyridazin-3-yl)amino)benzamide (18 mg, 41%) as a white solid.

Synthesis of Compound 215

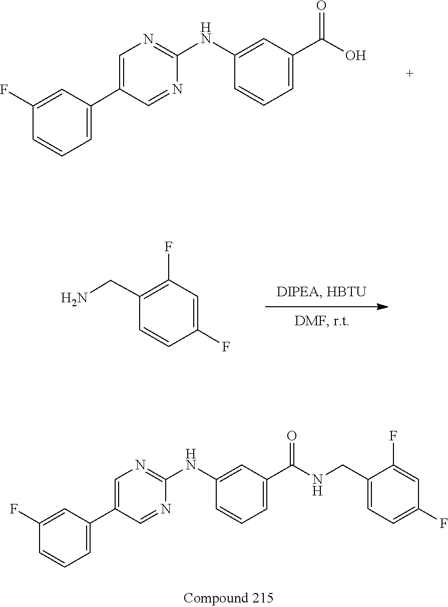 [SMILES: O=C(O)c1cccc(Nc2ncc(-c3cccc(F)c3)cn2)c1 ; NCc1ccc(F)cc1F ; O=C(NCc1ccc(F)cc1F)c1cccc(Nc2ncc(-c3cccc(F)c3)cn2)c1]

3-((5-(3-Fluorophenyl)pyrimidin-2-yl)amino)benzoic acid (50 mg, 0.16 mmol), (2,4-difluorophenyl)methanamine (0.02 mL, 0.19 mmol), and HBTU (92 mg, 0.24 mmol) were dissolved in DMF (2 mL), followed up by addition of DIPEA (0.04 mL, 0.24 mmol) and stirred for 20 hours at room temperature. The reaction mixture was concentrated under reduced pressure and the residue was dissolved in MeOH and sonicated. The slurry was filtered off and washed with MeOH to give compound 215, N-(2,4-difluorobenzyl)-3-((5-(3-fluorophenyl)pyrimidin-2-yl)amino)benzamide (58 mg, 83%) as an off-white solid.

Synthesis of Compound 216

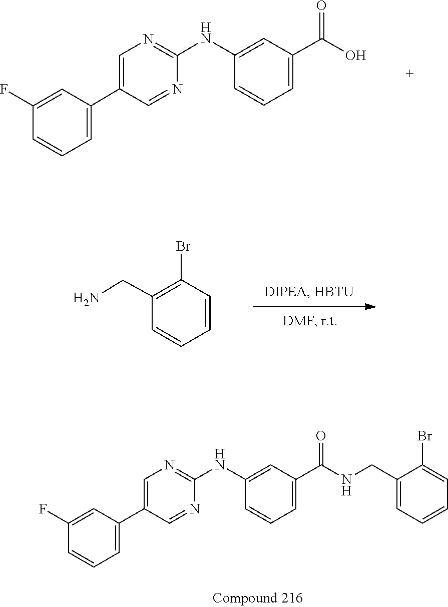 [SMILES: O=C(O)c1cccc(Nc2ncc(-c3cccc(F)c3)cn2)c1 ; NCc1ccccc1Br ; O=C(NCc1ccccc1Br)c1cccc(Nc2ncc(-c3cccc(F)c3)cn2)c1]

3-((5-(3-Fluorophenyl)pyrimidin-2-yl)amino)benzoic acid (50 mg, 0.16 mmol), (2-bromophenyl)methanamine (0.02 mL, 0.19 mmol), and HBTU (92 mg, 0.24 mmol) were dissolved in DMF (2 mL), followed up by addition of DIPEA (0.04 mL, 0.24 mmol) and stirred for 20 hours at room temperature. The reaction mixture was concentrated under reduced pressure and the residue dissolved in MeOH and sonicated. The slurry was filtered off and washed with MeOH to give compound 216, N-(2-bromobenzyl)-3-((5-(3-fluorophenyl)pyrimidin-2-yl)amino)benzamide (64 mg, 84%) as a white solid.

Synthesis of Compound 217

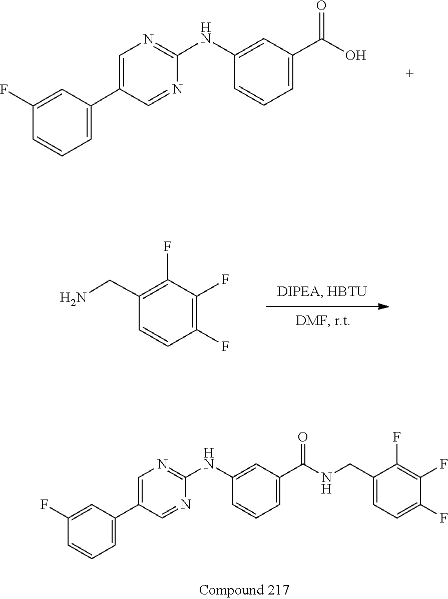 [SMILES: O=C(O)c1cccc(Nc2ncc(-c3cccc(F)c3)cn2)c1 ; NCc1ccc(F)c(F)c1F ; O=C(NCc1ccc(F)c(F)c1F)c1cccc(Nc2ncc(-c3cccc(F)c3)cn2)c1]

3-((5-(3-Fluorophenyl)pyrimidin-2-yl)amino)benzoic acid (50 mg, 0.16 mmol), (2,3,4-trifluorophenyl)methanamine (0.03 mL, 0.19 mmol), and HBTU (92 mg, 0.24 mmol) were dissolved in DMF (2 mL), followed up by addition of DIPEA (0.04 mL, 0.24 mmol) and stirred for 20 hours at room temperature. The reaction mixture was concentrated under reduced pressure and the residue was dissolved in MeOH and sonicated. The slurry was filtered off and washed with MeOH to give compound 217, 3-((5-(3-fluorophenyl)pyrimidin-2-yl)amino)-N-(2,3,4-trifluorobenzyl)benzamide (58 mg, 81%) as a white solid.

Synthesis of Compound 218

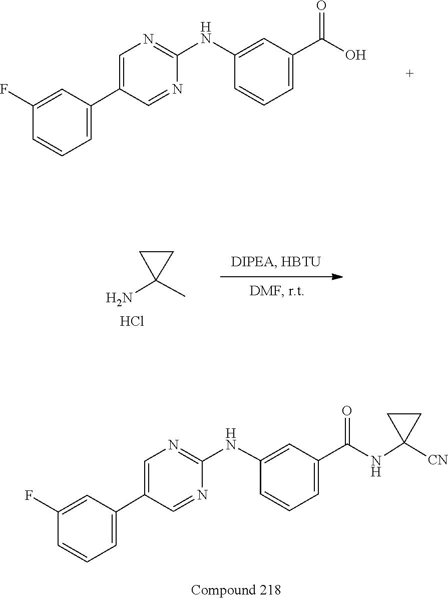

3-((5-(3-Fluorophenyl)pyrimidin-2-yl)amino)benzoic acid (50 mg, 0.16 mmol), 1-aminocyclopropane-1-carbonitrile hydrochloride (23 mg, 0.19 mmol), and HBTU (92 mg, 0.24 mmol) were dissolved in DMF (2 mL), followed up by addition of DIPEA (0.08 mL, 0.48 mmol) and stirred for 20 hours at room temperature. The reaction mixture was concentrated under reduced pressure and the residue was purified by MPLC. The crude mixture was solidified by using EA and HEX to give compound 218, N-(1-cyanocyclopropyl)-3-((5-(3-fluorophenyl)pyrimidin-2-yl)amino)benzamide (11 mg, 18%) as a white solid.

Synthesis of Compound 219

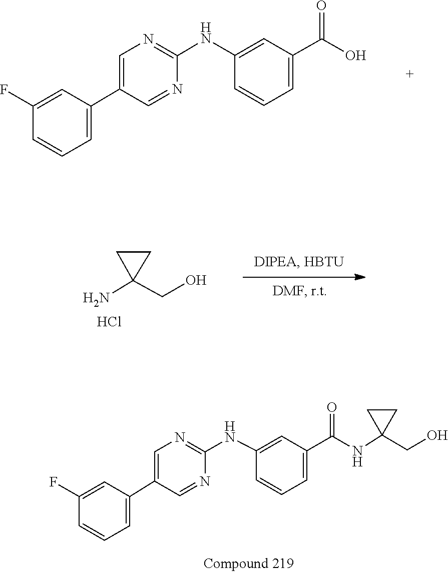 [SMILES: O=C(O)c1cccc(Nc2ncc(-c3cccc(F)c3)cn2)c1 ; NC1(CO)CC1 ; Cl ; O=C(NC1(CO)CC1)c1cccc(Nc2ncc(-c3cccc(F)c3)cn2)c1]

3-((5-(3-Fluorophenyl)pyrimidin-2-yl)amino)benzoic acid (50 mg, 0.16 mmol), (1-aminocyclopropyl)methanol hydrochloride (24 mg, 0.19 mmol), and HBTU (92 mg, 0.24 mmol) were dissolved in DMF (2 mL), followed up by addition of DIPEA (0.08 mL, 0.48 mmol) and stirred for 20 hours at room temperature. The reaction mixture was concentrated under reduced pressure and the residue was dissolved in MeOH and sonicated. The slurry was filtered off and washed with MeOH to give compound 219, 3-((5-(3-fluorophenyl)pyrimidin-2-yl)amino)-N-(1-(hydroxymethyl)cyclopropyl)benzamide (21 mg, 36%) as a white solid.

Synthesis of Compound 221

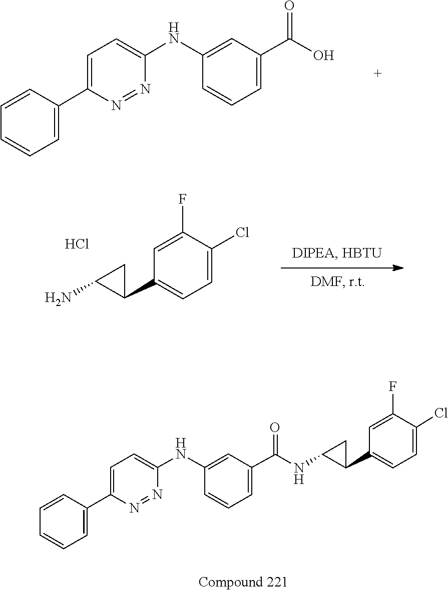 [SMILES: O=C(O)c1cccc(Nc2ccc(-c3ccccc3)nn2)c1 ; N[C@@H]1C[C@H]1c1ccc(Cl)c(F)c1 ; Cl ; O=C(N[C@@H]1C[C@H]1c1ccc(Cl)c(F)c1)c1cccc(Nc2ccc(-c3ccccc3)nn2)c1]

3-((6-Phenylpyridazin-3-yl)amino)benzoic acid (30 mg, 0.10 mmol), (1R,2S)-2-(4-chloro-3-fluorophenyl)cyclopropan-1-amine hydrochloride (28 mg, 0.12 mmol), and HBTU (59 mg, 0.16 mmol) were dissolved in DMF (1 mL), followed up by addition of DIPEA (0.05 mL, 0.31 mmol) and stirred for 22 hours at room temperature. The reaction mixture was extracted by EA and brine. The organic layer was dried over anhydrous Na₂SO₄ and concentrated. The residue was purified by MPLC. The crude mixture was solidified by using EA and HEX to give compound 221, N-((1R,2S)-2-(4-chloro-3-fluorophenyl)cyclopropyl)-3-((6-phenylpyridazin-3-yl)amino)benzamide (47 mg, 99%) as a white solid.

Synthesis of Compound 223

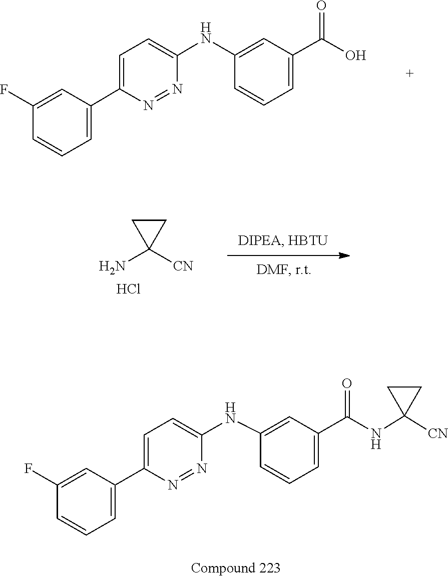 [SMILES: O=C(O)c1cccc(Nc2ccc(-c3cccc(F)c3)nn2)c1 ; N#CC1(N)CC1 ; Cl ; N#CC1(NC(=O)c2cccc(Nc3ccc(-c4cccc(F)c4)nn3)c2)CC1]

3-((6-(3-Fluorophenyl)pyridazin-3-yl)amino)benzoic acid (50 mg, 0.16 mmol), 1-aminocyclopropane-1-carbonitrile hydrochloride (23 mg, 0.19 mmol), and HBTU (92 mg, 0.24 mmol) were dissolved in DMF (2 mL), followed up by addition of DIPEA (0.08 mL, 0.48 mmol) and stirred for 20 hours at room temperature. The reaction mixture was concentrated under reduced pressure and the residue was purified by MPLC to give compound 223, N-(1-cyanocyclopropyl)-3-((6-(3-fluorophenyl)pyridazin-3-yl)amino)benzamide (19 mg, 31%) as a white solid.

Synthesis of Compound 224

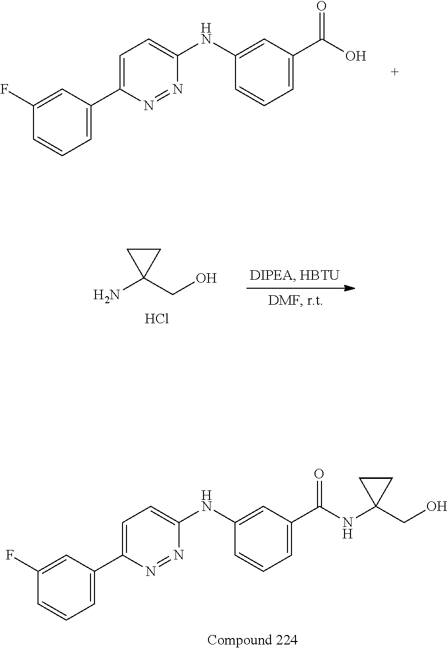 [SMILES: O=C(O)c1cccc(Nc2ccc(-c3cccc(F)c3)nn2)c1 ; NC1(CO)CC1 ; Cl ; O=C(NC1(CO)CC1)c1cccc(Nc2ccc(-c3cccc(F)c3)nn2)c1]

3-((6-(3-Fluorophenyl)pyridazin-3-yl)amino)benzoic acid (50 mg, 0.16 mmol), (1-aminocyclopropyl)methanol hydrochloride (24 mg, 0.19 mmol), and HBTU (92 mg, 0.24 mmol) were dissolved in DMF (2 mL), followed up by addition of DIPEA (0.08 mL, 0.48 mmol) and stirred for 20 hours at room temperature. The reaction mixture was concentrated under reduced pressure and the residue was purified by MPLC. The crude mixture was solidified by using EA and HEX to give compound 224, 3-((6-(3-fluorophenyl)pyridazin-3-yl)amino)-N-(1-(hydroxymethyl)cyclopropyl)benzamide (35 mg, 58%) as a white solid.

Synthesis of Compound 225

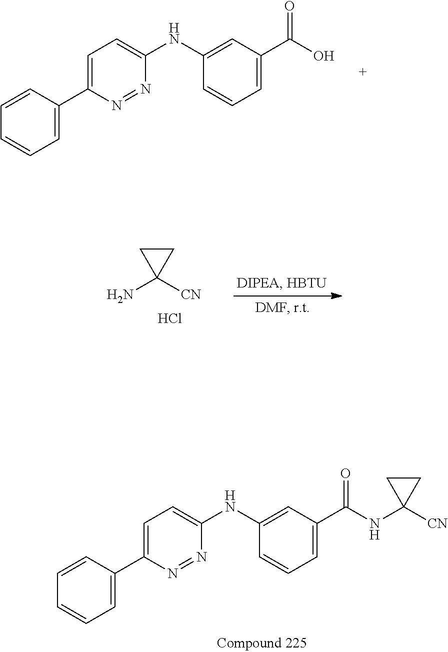 [SMILES: O=C(O)c1cccc(Nc2ccc(-c3ccccc3)nn2)c1 ; N#CC1(N)CC1 ; Cl ; N#CC1(NC(=O)c2cccc(Nc3ccc(-c4ccccc4)nn3)c2)CC1]

3-((6-Phenylpyridazin-3-yl)amino)benzoic acid (50 mg, 0.17 mmol), 1-aminocyclopropane-1-carbonitrile hydrochloride (24 mg, 0.21 mmol), and HBTU (98 mg, 0.26 mmol) were dissolved in DMF (2 mL), followed up by addition of DIPEA (0.09 mL, 0.51 mmol) and stirred for 20 hours at room temperature. The reaction mixture was concentrated under reduced pressure and the residue was purified by MPLC to give compound 225, N-(1-cyanocyclopropyl)-3-((6-phenylpyridazin-3-yl)amino)benzamide (21 mg, 35%) as a white solid.

Synthesis of Compound 228

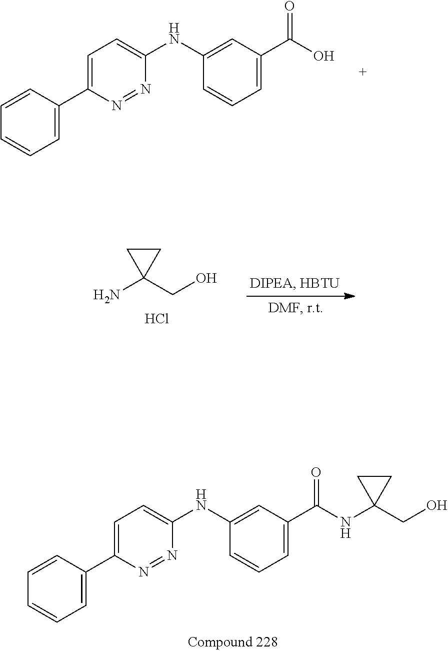 [SMILES: O=C(O)c1cccc(Nc2ccc(-c3ccccc3)nn2)c1 ; NC1(CO)CC1 ; Cl ; O=C(NC1(CO)CC1)c1cccc(Nc2ccc(-c3ccccc3)nn2)c1]

3-((6-Phenylpyridazin-3-yl)amino)benzoic acid (50 mg, 0.17 mmol), (1-aminocyclopropyl)methanol hydrochloride (25 mg, 0.21 mmol), and HBTU (98 mg, 0.26 mmol) were dissolved in DMF (2 mL), followed up by addition of DIPEA (0.09 mL, 0.51 mmol) and stirred for 20 hours at room temperature. The reaction mixture was concentrated under reduced pressure and the residue was purified by MPLC to give compound 228, N-(1-(hydroxymethyl)cyclopropyl)-3-((6-phenylpyridazin-3-yl)amino)benzamide (29 mg, 47%) as a white solid.

Synthesis of Compound 229

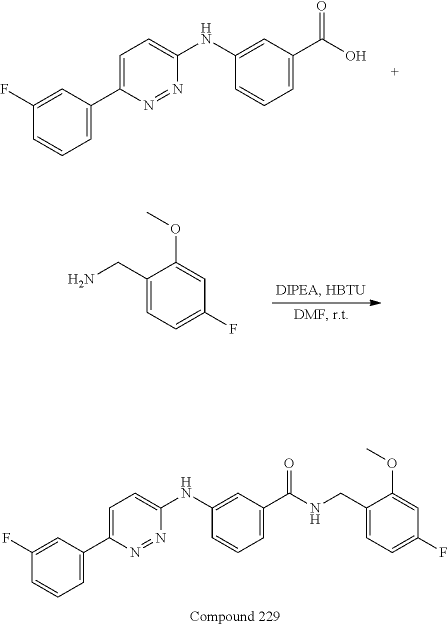 [SMILES: O=C(O)c1cccc(Nc2ccc(-c3cccc(F)c3)nn2)c1 ; COc1cc(F)ccc1CN ; COc1cc(F)ccc1CNC(=O)c1cccc(Nc2ccc(-c3cccc(F)c3)nn2)c1]

3-((6-(3-Fluorophenyl)pyridazin-3-yl)amino)benzoic acid (300 mg, 0.97 mmol), (4-fluoro-2-methoxyphenyl)methanamine (166 mg, 1.07 mmol), and HBTU (405 mg, 1.07 mmol) were dissolved in DCM (10 mL), followed up by addition of DIPEA (0.33 mL, 1.94 mmol) and stirred for 16 hours at room temperature. The reaction mixture was extracted by DCM and H₂O. The organic layer was dried over anhydrous Na₂SO₄ and concentrated. The residue was purified by MPLC. The crude mixture was solidified by using EA and HEX to give compound 229, N-(4-fluoro-2-methoxybenzyl)-3-((6-(3-fluorophenyl)pyridazin-3-yl)amino)benzamide (435 mg, >100%) as a yellow solid.

Synthesis of Compound 230

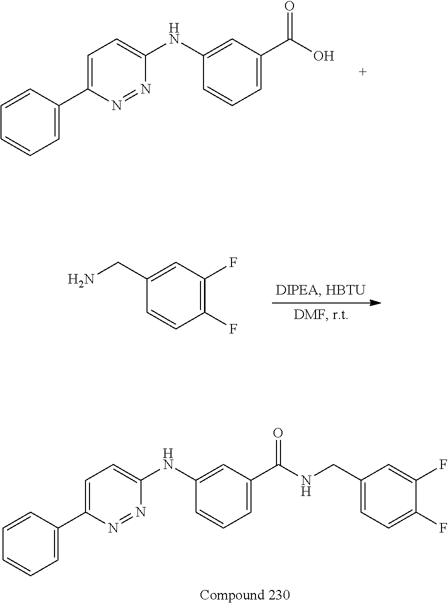 [SMILES: O=C(O)c1cccc(Nc2ccc(-c3ccccc3)nn2)c1 ; NCc1ccc(F)c(F)c1 ; O=C(NCc1ccc(F)c(F)c1)c1cccc(Nc2ccc(-c3ccccc3)nn2)c1]

3-((6-Phenylpyridazin-3-yl)amino)benzoic acid (30 mg, 0.10 mmol), (3,4-difluorophenyl)methanamine (0.02 mL, 0.12 mmol), and HBTU (59 mg, 0.16 mmol) were dissolved in DMF (1 mL), followed up by addition of DIPEA (0.05 mL, 0.31 mmol) and stirred for 4 hours at room temperature. The reaction mixture was extracted by EA and brine. The organic layer was dried over anhydrous Na₂SO₄ and concentrated. The residue was purified by MPLC. The crude mixture was solidified by using EA and HEX to give compound 230, N-(3,4-difluorobenzyl)-3-((6-phenylpyridazin-3-yl)amino)benzamide (14 mg, 31%) as a white solid.

Synthesis of Compound 231

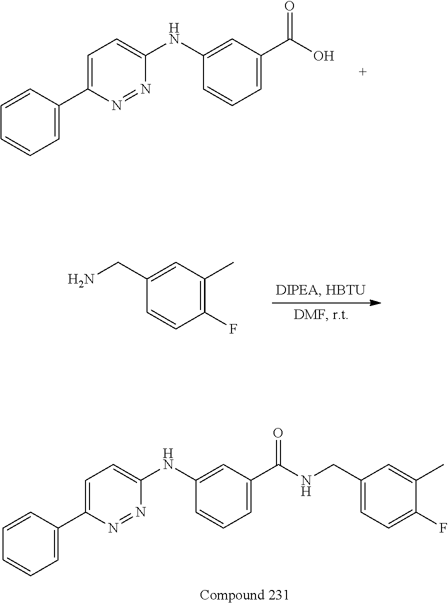

3-((6-Phenylpyridazin-3-yl)amino)benzoic acid (30 mg, 0.10 mmol), (4-fluoro-3-methylphenyl)methanamine (17 mg, 0.12 mmol), and HBTU (59 mg, 0.16 mmol) were dissolved in DMF (1 mL), followed up by addition of DIPEA (0.05 mL, 0.31 mmol) and stirred for 4 hours at room temperature. The reaction mixture was extracted by EA and brine. The organic layer was dried over anhydrous Na₂SO₄ and concentrated. The residue was purified by MPLC. The crude mixture was solidified by using EA and HEX to give compound 231, N-(4-fluoro-3-methylbenzyl)-3-((6-phenylpyridazin-3-yl)amino)benzamide (11 mg, 25%) as a white solid.

Synthesis of Compound 232

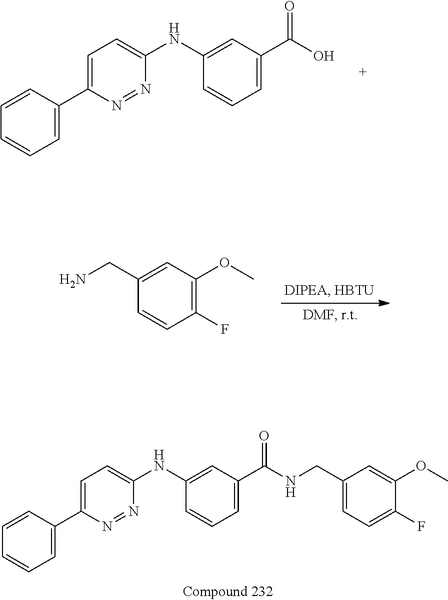 [SMILES: O=C(O)c1cccc(Nc2ccc(-c3ccccc3)nn2)c1 ; COc1cc(CN)ccc1F ; COc1cc(CNC(=O)c2cccc(Nc3ccc(-c4ccccc4)nn3)c2)ccc1F]

3-((6-Phenylpyridazin-3-yl)amino)benzoic acid (30 mg, 0.10 mmol), (4-fluoro-3-methoxyphenyl)methanamine (0.02 mL, 0.12 mmol), and HBTU (59 mg, 0.16 mmol) were dissolved in DMF (1 mL), followed up by addition of DIPEA (0.05 mL, 0.31 mmol) and stirred for 4 hours at room temperature. The reaction mixture was extracted by EA and brine. The organic layer was dried over anhydrous Na₂SO₄ and concentrated. The residue was purified by MPLC. The crude mixture was solidified by using EA and HEX to give compound 232, N-(4-fluoro-3-methoxybenzyl)-3-((6-phenylpyridazin-3-yl)amino)benzamide (39 mg, 89%) as a white solid.

Synthesis of Compound 233

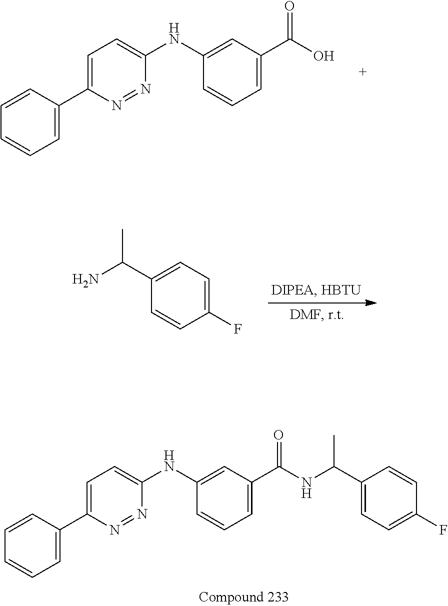

3-((6-Phenylpyridazin-3-yl)amino)benzoic acid (30 mg, 0.10 mmol), 1-(4-fluorophenyl)ethan-1-amine (0.02 mL, 0.12 mmol), and HBTU (59 mg, 0.16 mmol) were dissolved in DMF (1 mL), followed up by addition of DIPEA (0.05 mL, 0.31 mmol) and stirred for 4 hours at room temperature. The reaction mixture was extracted by EA and brine. The organic layer was dried over anhydrous Na₂SO₄ and concentrated. The residue was purified by MPLC. The crude mixture was solidified by using EA and HEX to give compound 233, N-(1-(4-fluorophenyl)ethyl)-3-((6-phenylpyridazin-3-yl)amino)benzamide (20 mg, 47%) as a white solid.

Synthesis of Compound 234

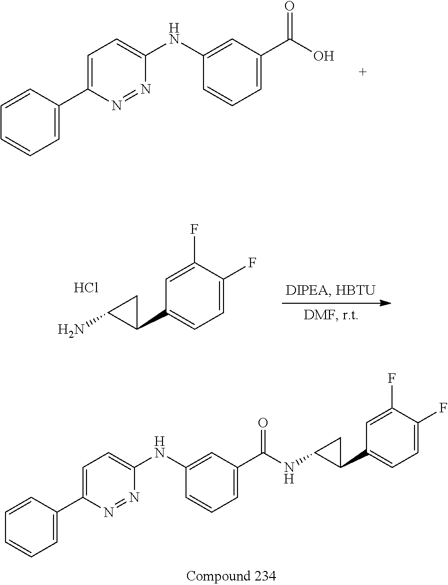 [SMILES: O=C(O)c1cccc(Nc2ccc(-c3ccccc3)nn2)c1 ; N[C@@H]1C[C@H]1c1ccc(F)c(F)c1 ; Cl ; O=C(N[C@@H]1C[C@H]1c1ccc(F)c(F)c1)c1cccc(Nc2ccc(-c3ccccc3)nn2)c1]

3-((6-Phenylpyridazin-3-yl)amino)benzoic acid (30 mg, 0.10 mmol), (1R,2S)-2-(3,4-difluorophenyl)cyclopropan-1-amine hydrochloride (26 mg, 0.12 mmol), and HBTU (59 mg, 0.16 mmol) were dissolved in DMF (1 mL), followed up by addition of DIPEA (0.05 mL, 0.31 mmol) and stirred for 25 hours at room temperature. The reaction mixture was extracted by EA and brine. The organic layer was dried over anhydrous Na₂SO₄ and concentrated. The residue was purified by MPLC. The crude mixture was solidified by using EA and HEX to give compound 234, N-((1R,2S)-2-(3,4-difluorophenyl)cyclopropyl)-3-((6-phenylpyridazin-3-yl)amino)benzamide (40 mg, 87%) as a white solid.

Synthesis of Compound 235

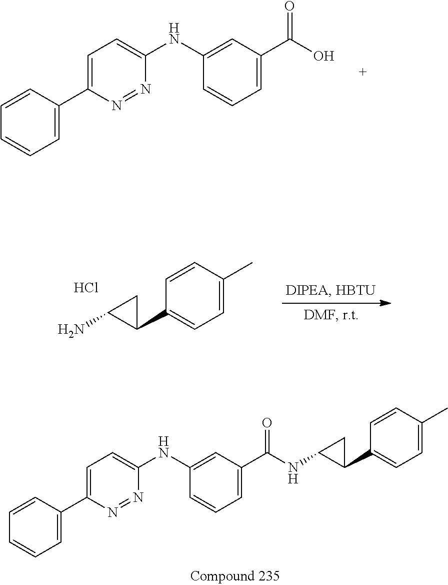 [SMILES: O=C(O)c1cccc(Nc2ccc(-c3ccccc3)nn2)c1 ; Cc1ccc([C@@H]2C[C@H]2N)cc1 ; Cl ; Cc1ccc([C@@H]2C[C@H]2NC(=O)c2cccc(Nc3ccc(-c4ccccc4)nn3)c2)cc1]

3-((6-Phenylpyridazin-3-yl)amino)benzoic acid (30 mg, 0.10 mmol), (UR 2S)-2-(p-tolyl)cyclopropan-1-amine (23 mg, 0.12 mmol), and HBTU (59 mg, 0.16 mmol) were dissolved in DMF (1 mL), followed up by addition of DIPEA (0.05 mL, 0.31 mmol) and stirred for 25 hours at room temperature. The reaction mixture was extracted by EA and brine. The organic layer was dried over anhydrous Na₂SO₄ and concentrated. The residue was purified by MPLC. The crude mixture was solidified by using EA and HEX to give compound 235, 3-((6-phenylpyridazin-3-yl)amino)-N-((1R,2S)-2-(p-tolyl)cyclopropyl)benzamide (36 mg, 84%) as a white solid.

Synthesis of Compound 236

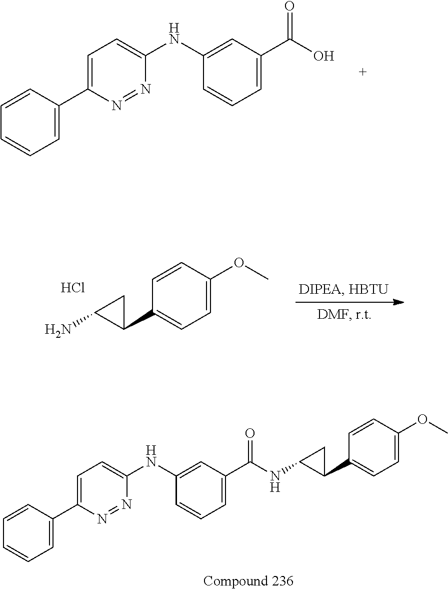 [SMILES: O=C(O)c1cccc(Nc2ccc(-c3ccccc3)nn2)c1 ; COc1ccc([C@@H]2C[C@H]2N)cc1 ; Cl ; COc1ccc([C@@H]2C[C@H]2NC(=O)c2cccc(Nc3ccc(-c4ccccc4)nn3)c2)cc1]

3-((6-Phenylpyridazin-3-yl)amino)benzoic acid (30 mg, 0.10 mmol), (1R,2S)-2-(4-methoxyphenyl)cyclopropan-1-amine (25 mg, 0.12 mmol), and HBTU (59 mg, 0.16 mmol) were dissolved in DMF (1 mL), followed up by addition of DIPEA (0.05 mL, 0.31 mmol) and stirred for 25 hours at room temperature. The reaction mixture was extracted by EA and aq. NH₄Cl. The organic layer was dried over anhydrous Na₂SO₄ and concentrated. The residue was purified by MPLC. The crude mixture was solidified by using EA and HEX to give compound 236, N-((1R,2S)-2-(4-methoxyphenyl)cyclopropyl)-3-((6-phenylpyridazin-3-yl)amino)benzamide (25 mg, 56%) as a white solid.

Synthesis of Compound 237

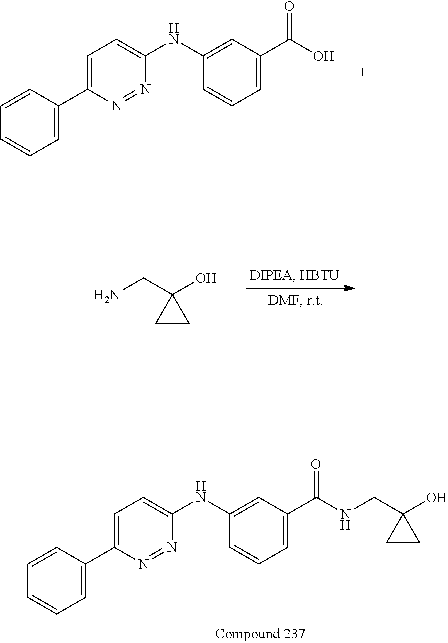 [SMILES: O=C(O)c1cccc(Nc2ccc(-c3ccccc3)nn2)c1 ; NCC1(O)CC1 ; O=C(NCC1(O)CC1)c1cccc(Nc2ccc(-c3ccccc3)nn2)c1]

3-((6-Phenylpyridazin-3-yl)amino)benzoic acid (50 mg, 0.17 mmol), 1-(aminomethyl)cyclopropan-1-ol (18 mg, 0.21 mmol), and HBTU (98 mg, 0.26 mmol) were dissolved in DMF (2 mL), followed up by addition of DIPEA (0.04 mL, 0.26 mmol) and stirred for 23 hours at room temperature. The reaction mixture was concentrated under reduced pressure and the residue was purified by MPLC. The crude mixture was solidified by using EA and HEX to give compound 237, N-((1-hydroxycyclopropyl)methyl)-3-((6-phenylpyridazin-3-yl)amino)benzamide (15 mg, 24%) as a white solid.

3. Synthesis by Method C

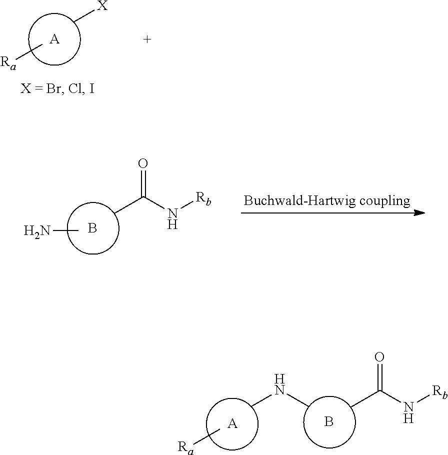

Synthesis of Compound 17

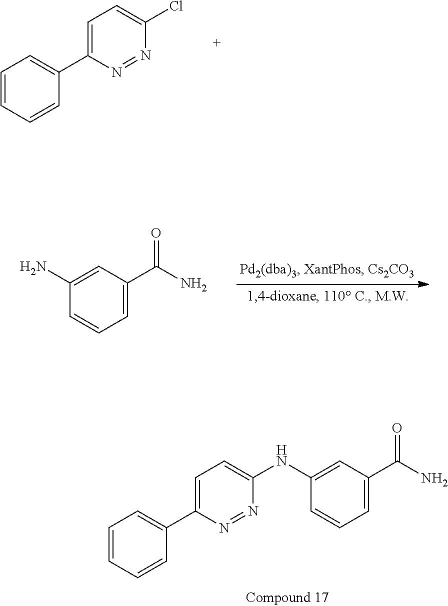 [SMILES: Clc1ccc(-c2ccccc2)nn1 ; NC(=O)c1cccc(N)c1 ; NC(=O)c1cccc(Nc2ccc(-c3ccccc3)nn2)c1]

3-Chloro-6-phenylpyridazine (75 mg, 0.39 mmol), 3-aminobenzamide (54 mg, 0.39 mmol), Pd₂(dba)₃ (36 mg, 0.039 mmol), XantPhos (46 mg, 0.079 mmol), and cesium carbonate (256 mg, 0.79 mmol) were mixed in 1,4-dioxane (2 mL) and heated in a microwave reactor for 60 minutes at 110° C. The reaction mixture was concentrated and purified by MPLC to give compound 17, 3-[(6-phenylpyridazin-3-yl)amino]benzamide (10 mg, 9%) as a white solid.

4. Synthesis by Method D

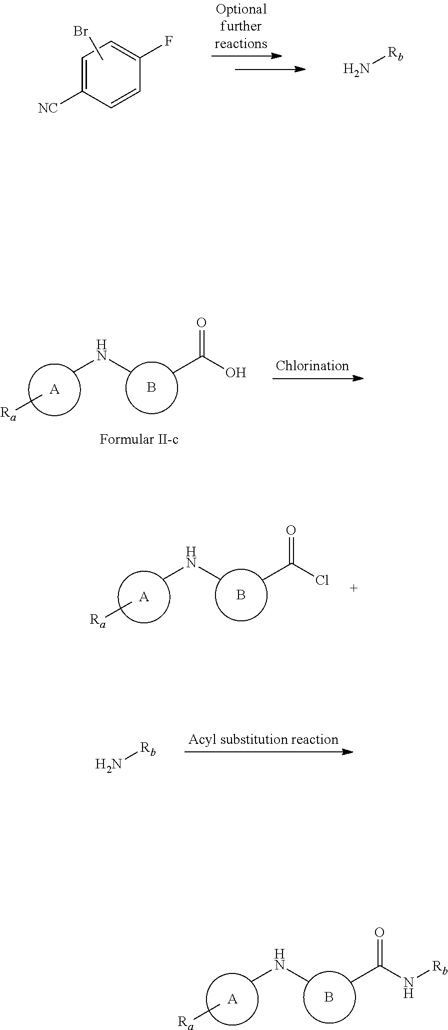

Synthesis of Compound 220

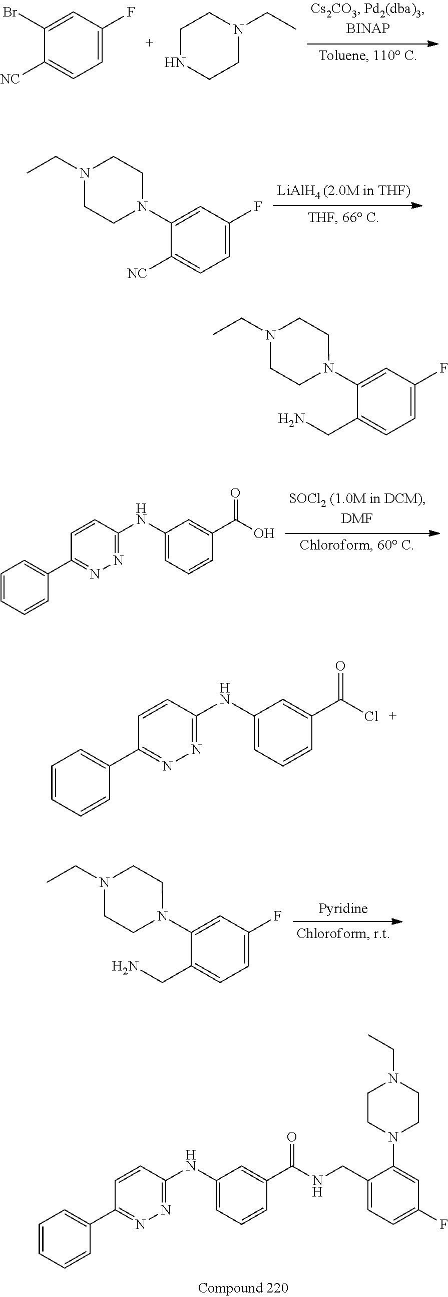

Step 1: 2-Bromo-4-fluorobenzonitrile (300 mg, 1.50 mmol), 1-ethylpiperazine (0.23 mL, 1.80 mmol), cesium carbonate (977 mg, 3.00 mmol), Pd₂(dba)₃ (137 mg, 0.15 mmol), and 2,2′-bis(diphenylphosphino)-1,1′-binaphthyl (BINAP) (93 mg, 0.15 mmol) were mixed in Toluene (15 mL) and stirred for 21 hours at 110° C. The reaction mixture was extracted by EA and brine. The organic layer was dried over anhydrous Na₂SO₄ and concentrated. The residue was purified by MPLC to give 2-(4-ethylpiperazin-1-yl)-4-fluorobenzonitrile (294 mg, 84%) as a pale-yellow solid.

Step 2: 2-(4-Ethylpiperazin-1-yl)-4-fluorobenzonitrile (290 mg, 1.24 mmol) was dissolved in THF (12 mL) followed up by dropwise addition of LiAlH₄ (2.0 M in THF) (1.87 mL, 3.73 mmol) at 0° C. Then the reaction mixture was stirred for 5 hours at 66° C. The reaction mixture was extracted by EA and aq. NaHCO₃ and concentrated to give (2-(4-ethylpiperazin-1-yl)-4-fluorophenyl)methanamine (212 mg, 72%) as a yellow liquid.

Step 3: To a solution of 3-((6-phenylpyridazin-3-yl)amino)benzoic acid (60 mg, 0.21 mmol) in chloroform (2 mL), DMF (catalytic amount), and SOCl₂ (1.0 M in DCM) (1.03 mL, 1.03 mmol) were added and stirred for 5 hours at 60° C. The mixture was concentrated to give 3-((6-phenylpyridazin-3-yl)amino)benzoyl chloride (64 mg, >100%) as a yellow solid.

Step 4: To a solution of (2-(4-ethylpiperazin-1-yl)-4-fluorophenyl)methanamine (49 mg, 0.21 mmol) and pyridine (0.05 mL, 0.62 mmol) in chloroform (2 mL), 3-((6-phenylpyridazin-3-yl)amino)benzoyl chloride (64 mg, 0.21 mmol) dissolved in chloroform (2 mL) was added dropwise and stirred for 19 hours at room temperature. The reaction mixture was extracted by DCM and aq. NH₄Cl. The organic layer was dried over anhydrous Na₂SO₄ and concentrated. The residue was purified by MPLC. The crude mixture was solidified by using EA and HEX to give compound 220, N-(2-(4-ethylpiperazin-1-yl)-4-fluorobenzyl)-3-((6-phenylpyridazin-3-yl)amino)benzamide (12 mg, 11%) as a white solid.

Synthesis of Compound 222

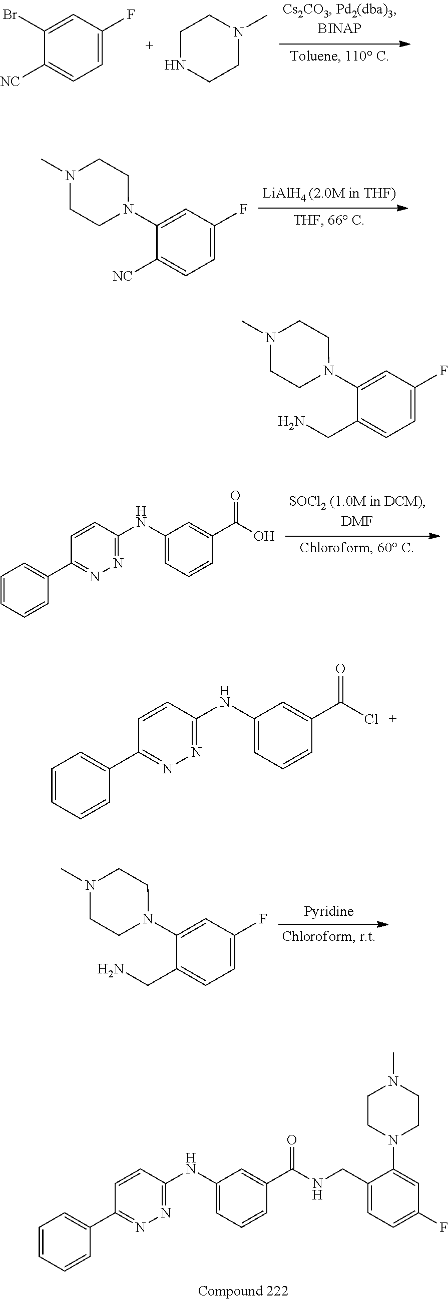

Step 1: 2-Bromo-4-fluorobenzonitrile (300 mg, 1.50 mmol), 1-methylpiperazine (0.20 mL, 1.80 mmol), cesium carbonate (977 mg, 3.00 mmol), Pd₂(dba)₃ (137 mg, 0.15 mmol), and BINAP (93 mg, 0.15 mmol) were mixed in Toluene (15 mL) and stirred for 17 hours at 110° C. The reaction mixture was extracted by EA and brine. The organic layer was dried over anhydrous Na₂SO₄ and concentrated. The residue was purified by MPLC to give 4-fluoro-2-(4-methylpiperazin-1-yl)benzonitrile (266 mg, 81%) as a pale yellow solid.

Step 2: 4-Fluoro-2-(4-methylpiperazin-1-yl)benzonitrile (266 mg, 1.22 mmol) was dissolved in THF (12 mL) followed up by dropwise addition of LiAlH₄ (2.0 M in THF) (1.82 mL, 3.64 mmol) at 0° C. Then the reaction mixture was stirred for 3 hours at 66° C. The reaction mixture was extracted by DCM and aq. NaHCO₃ and concentrated to give (4-fluoro-2-(4-methylpiperazin-1-yl)phenyl)methanamine (249 mg, 92%) as a brown liquid.

Step 3: To a solution of 3-((6-phenylpyridazin-3-yl)amino)benzoic acid (100 mg, 0.34 mmol) in chloroform (3 mL), DMF (catalytic amount), and SOCl₂ (1.0 M in DCM) (1.72 mL, 1.72 mmol) were added and stirred for 4 hours at 60° C. The mixture was concentrated to give 3-((6-phenylpyridazin-3-yl)amino)benzoyl chloride (106 mg, >100%) as a yellow solid.

Step 4: To a solution of (4-fluoro-2-(4-methylpiperazin-1-yl)phenyl)methanamine (77 mg, 0.34 mmol) and pyridine (0.08 mL, 1.03 mmol) in chloroform (3 mL), 3-((6-phenylpyridazin-3-yl)amino)benzoyl chloride (106 mg, 0.34 mmol) dissolved in chloroform (3 mL) was added dropwise and stirred for 4 hours at room temperature. The reaction mixture was extracted by DCM and aq. NaHCO₃. The organic layer was dried over anhydrous Na₂SO₄ and concentrated. The residue was purified by MPLC. The crude mixture was solidified by using EA and HEX to give compound 222, N-(4-fluoro-2-(4-methylpiperazin-1-yl)benzyl)-3-((6-phenylpyridazin-3-yl)amino)benzamide (25 mg, 15%) as a pale yellow solid.

Synthesis of Compound 226

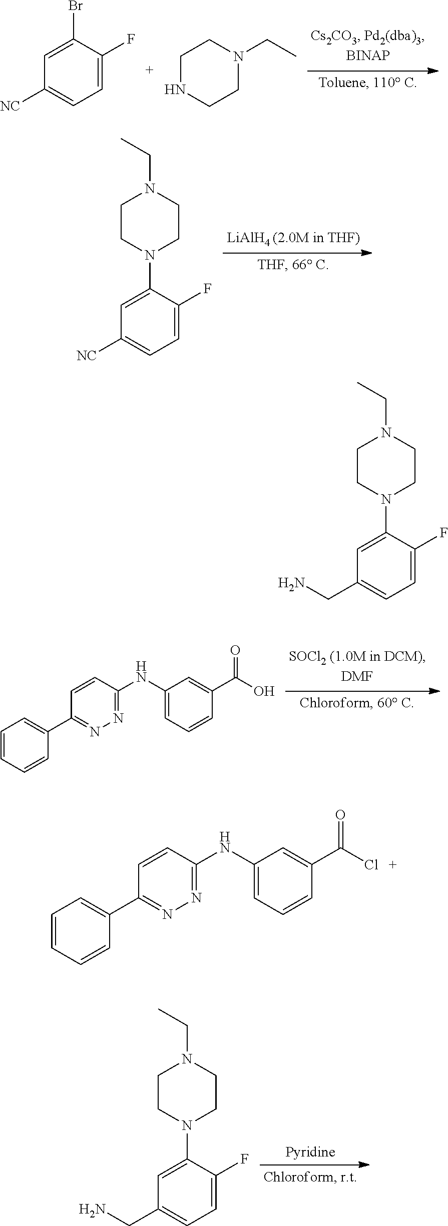

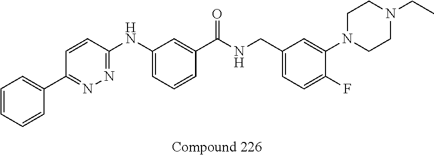 [SMILES: CCN1CCN(c2cc(CNC(=O)c3cccc(Nc4ccc(-c5ccccc5)nn4)c3)ccc2F)CC1]

Step 1: 3-Bromo-4-fluorobenzonitrile (300 mg, 1.50 mmol), 1-ethylpiperazine (0.23 mL, 1.80 mmol), cesium carbonate (977 mg, 3.00 mmol), Pd₂(dba)₃ (137 mg, 0.15 mmol), and BINAP (93 mg, 0.15 mmol) were mixed in Toluene (15 mL) and stirred for 18 hours at 110° C. The reaction mixture was extracted by EA and brine. The organic layer was dried over anhydrous Na₂SO₄ and concentrated. The residue was purified by MPLC to give 3-(4-ethylpiperazin-1-yl)-4-fluorobenzonitrile (278 mg, 79%) as a pale yellow solid.

Step 2: 3-(4-Ethylpiperazin-1-yl)-4-fluorobenzonitrile (277 mg, 1.19 mmol) was dissolved in THF (12 mL) followed up by dropwise addition of LiAlH₄ (2.0 M in THF) (1.78 mL, 3.56 mmol) at 0° C. Then the reaction mixture was stirred for 4 hours at 66° C. The reaction mixture was extracted by EA and aq. NaHCO₃ and concentrated to give (3-(4-ethylpiperazin-1-yl)-4-fluorophenyl)methanamine (186 mg, 66%) as a brown liquid.

Step 3: To a solution of 3-((6-phenylpyridazin-3-yl)amino)benzoic acid (100 mg, 0.34 mmol) in chloroform (3 mL), DMF (catalytic amount), and SOCl₂ (1.0 M in DCM) (1.72 mL, 1.72 mmol) were added and stirred for 8 hours at 60° C. The mixture was concentrated to give 3-((6-phenylpyridazin-3-yl)amino)benzoyl chloride (106 mg, >100%) as a yellow solid.

Step 4: To a solution of (3-(4-ethylpiperazin-1-yl)-4-fluorophenyl)methanamine (81 mg, 0.34 mmol) and pyridine (0.08 mL, 1.03 mmol) in chloroform (3 mL), 3-((6-phenylpyridazin-3-yl)amino)benzoyl chloride (106 mg, 0.34 mmol) dissolved in chloroform (3 mL) was added dropwise and stirred for 17 hours at room temperature. The reaction mixture was extracted by DCM and aq. NaHCO₃. The organic layer was dried over anhydrous Na₂SO₄ and concentrated. The residue was purified by MPLC. The crude mixture was solidified by using EA and HEX to give compound 226, N-(3-(4-ethylpiperazin-1-yl)-4-fluorobenzyl)-3-((6-phenylpyridazin-3-yl)amino)benzamide (4 mg, 2%) as a pale yellow solid.

Synthesis of Compound 227

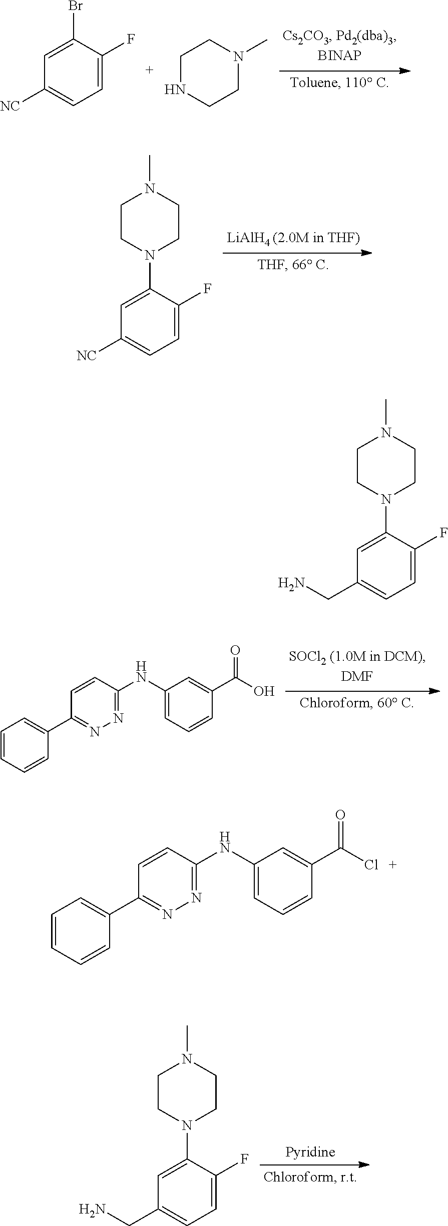

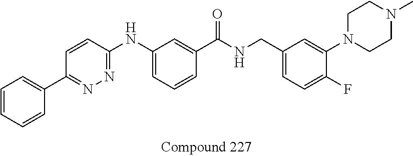 [SMILES: CN1CCN(c2cc(CNC(=O)c3cccc(Nc4ccc(-c5ccccc5)nn4)c3)ccc2F)CC1]

Step 1: 3-Bromo-4-fluorobenzonitrile (300 mg, 1.50 mmol), 1-methylpiperazine (0.20 mL, 1.80 mmol), cesium carbonate (977 mg, 3.00 mmol), Pd₂(dba)₃ (137 mg, 0.15 mmol), and BINAP (93 mg, 0.15 mmol) were mixed in Toluene (15 mL) and stirred for 18 hours at 110° C. The reaction mixture was extracted by EA and brine. The organic layer was dried over anhydrous Na₂SO₄ and concentrated. The residue was purified by MPLC to give 4-fluoro-3-(4-methylpiperazin-1-yl)benzonitrile (208 mg, 63%) as a pale yellow solid.

Step 2: 4-Fluoro-3-(4-methylpiperazin-1-yl)benzonitrile (207 mg, 0.94 mmol) was dissolved in THF (9 mL) followed up by dropwise addition of LiAlH₄ (2.0 M in THF) (1.42 mL, 2.83 mmol) at 0° C. Then the reaction mixture was stirred for 4 hours at 66° C. The reaction mixture was extracted by EA and aq. NaHCO₃ and concentrated to (4-fluoro-3-(4-methylpiperazin-1-yl)phenyl)methanamine (143 mg, 68%) as a brown liquid.

Step 3: To a solution of 3-((6-phenylpyridazin-3-yl)amino)benzoic acid (100 mg, 0.34 mmol) in chloroform (3 mL), DMF (catalytic amount), and SOCl₂ (1.0 M in DCM) (1.72 mL, 1.72 mmol) were added and stirred for 8 hours at 60° C. The mixture was concentrated to give 3-((6-phenylpyridazin-3-yl)amino)benzoyl chloride (106 mg, >100%) as a yellow solid.

Step 4: To a solution of (4-fluoro-3-(4-methylpiperazin-1-yl)phenyl)methanamine (77 mg, 0.34 mmol) and pyridine (0.08 mL, 1.03 mmol) in chloroform (3 mL), 3-((6-phenylpyridazin-3-yl)amino)benzoyl chloride (106 mg, 0.34 mmol) dissolved in chloroform (3 mL) was added dropwise and stirred for 17 hours at room temperature. The reaction mixture was extracted by DCM and aq. NaHCO₃. The organic layer was dried over anhydrous Na₂SO₄ and concentrated. The residue was purified by MPLC. The crude mixture was solidified by using EA and HEX to give compound 227, N-(4-fluoro-3-(4-methylpiperazin-1-yl)benzyl)-3-((6-phenylpyridazin-3-yl)amino)benzamide (6 mg, 3%) as a white solid.

5. Synthesis by Method E

Synthesis of Compound 180

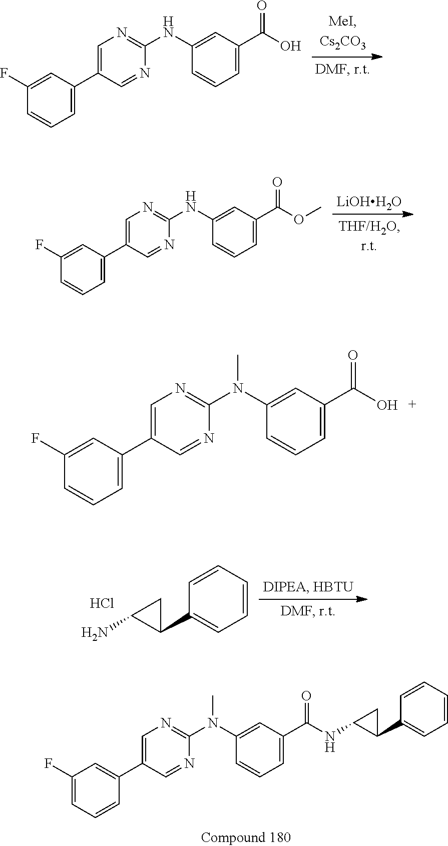

Step 1: 3-((5-(3-Fluorophenyl)pyrimidin-2-yl)amino)benzoic acid (618 mg, 2 mmol) and cesium carbonate (1,955 mg, 6 mmol) were mixed in DMF (10 mL) followed up by addition of methyl iodide (0.274 mL, 2.2 mmol) and stirred for 5 days at room temperature. The reaction mixture was concentrated and purified by MPLC. The crude mixture was solidified by using DCM to give methyl 3-((5-(3-fluorophenyl)pyrimidin-2-yl)(methyl)amino)benzoate (512 mg, 76%) as a beige solid.

Step 2: Methyl 3-((5-(3-fluorophenyl)pyrimidin-2-yl)(methyl)amino)benzoate (400 mg, 1.18 mmol) and LiOH.H₂O (496 mg, 11.8 mmol) were mixed in H₂O/THF (5/10 mL) and stirred for 8 hours at room temperature. The reaction mixture acidified by adding 1 N HCl and the suspension was filtered. The filter cake was washed with H₂O (100 mL) and dried under vacuum to give 3-((5-(3-fluorophenyl)pyrimidin-2-yl)(methyl)amino)benzoic acid (369 mg, 97%) as a white solid.

Step 3: 3-((5-(3-Fluorophenyl)pyrimidin-2-yl)(methyl)amino)benzoic acid (100 mg, 0.35 mmol), (1R,2S)-2-phenylcyclopropan-1-amine hydrochloride (58 mg, 0.34 mmol) and HBTU (176 mg, 0.46 mmol) were dissolved in DMF (3.1 mL), followed up by addition of DIPEA (0.16 mL, 0.93 mmol) and stirred for 16 hours at room temperature. The reaction mixture was extracted by EA and brine. The organic layer was dried over anhydrous Na₂SO₄ and concentrated. The residue was solidified by using EA and HEX to give compound 180, 3-((5-(3-fluorophenyl)pyrimidin-2-yl)(methyl)amino)-N-((1R,2S)-2-phenylcyclopropyl)benzamide (90 mg, 67%) as a beige solid.

Synthesis of Compound 185

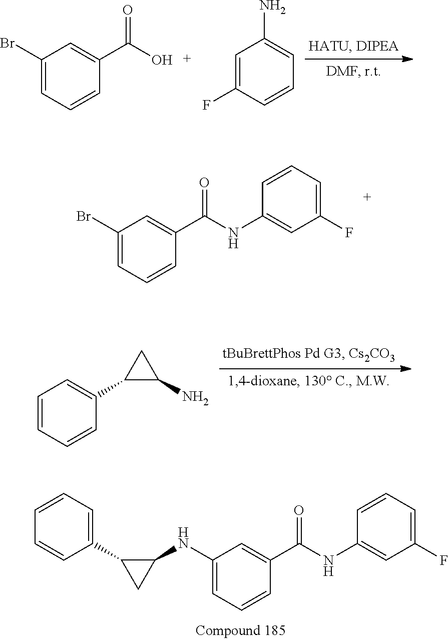

Step 1: 3-Bromobenzoic acid (0.2 g, 0.995 mmol) and hexafluorophosphate azabenzotriazole tetramethyl uronium (0.57 g, 1.492 mmol) in DMF (2 mL) was added DIPEA (0.52 mL, 2.984 mmol) at room temperature. After 15 minutes of stirring, 3-flouroaniline (0.133 g, 1.193 mmol) was added and the reaction mixture was stirred for 2 hours at room temperature. The reaction mixture was extracted by EA and water. The organic layer was dried over anhydrous Na₂SO₄ and concentrated. The residue was purified by MPLC to give 3-bromo-N-(3-fluorophenyl)benzamide (160 mg, 55%).

Step 2: 3-Bromo-N-(3-fluorophenyl)benzamide (0.205 g, 0.697 mmol) and (1R,2S)-2-phenylcyclopropan-1-amine (0.102 g, 0.767 mmol), t-Butyl BrettPhos Pd G3 (0.032 g, 0.035 mmol), and cesium carbonate (0.68 g, 2.091 mmol) were mixed in 1,4-dioxane (2 mL) and heated in a microwave reactor for 2 hours at 130° C. The reaction mixture was poured into water, and extracted by EA. The organic layer was dried over anhydrous Na₂SO₄ and concentrated. The residue was purified by prep. HPLC to give compound 185, N-(3-fluorophenyl)-3-(((1R,2S)-2-phenylcyclopropyl)amino)benzamide (24 mg, 10%) as a white solid.

Synthesis of Compound 186

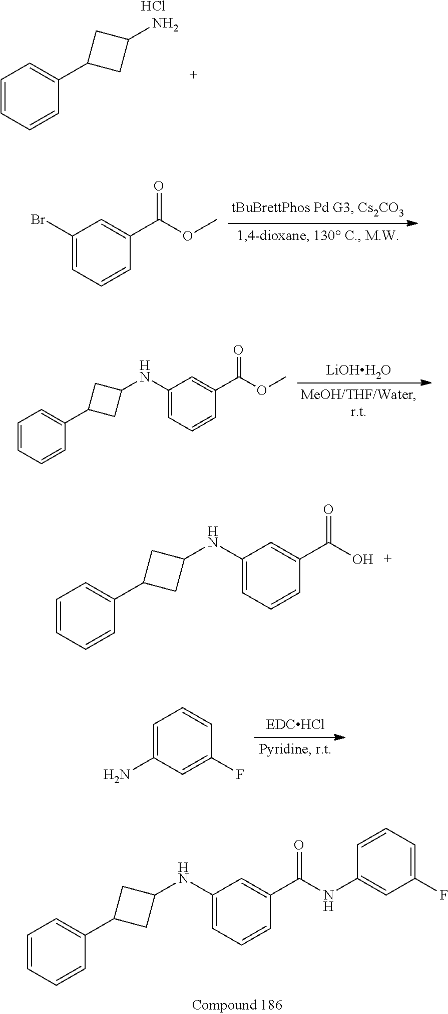

Step 1: 3-Phenylcyclobutan-1-amine HCl salt (0.475 g, 2.586 mmol), methyl 3-bromobenzoate (0.612 g, 2.845 mmol), t-Butyl BrettPhos Pd G3 (117 mg, 0.129 mmol), and cesium carbonate (2.528 g, 7.758 mmol) were mixed in 1,4-dioxane (9.5 mL) and heated in a microwave reactor for 1 hour at 130° C. The reaction mixture was poured into water, and extracted by EA. The organic layer was dried over anhydrous Na₂SO₄ and concentrated. The residue was purified by MPLC to give methyl 3-((3-phenylcyclobutyl)amino)benzoate (0.41 g, 56%).

Step 2: Methyl 3-((3-phenylcyclobutyl)amino)benzoate (0.40 g, 1.421 mmol) and LiOH.H₂O (0.24 g, 5.7 mmol) were mixed in MeOH/THF/H₂O (1/2/1 mL) and stirred for 4 hours at room temperature. The reaction mixture acidified by adding 1 N HCl, precipitates were filtered and washed with water to give 3-((3 phenylcyclobutyl)amino)benzoic acid (0.23 g, 61%).

Step 3: 3-((3-Phenylcyclobutyl)amino)benzoic acid (0.23 g, 0.860 mmol), 3-flouroaniline (0.105 g, 0.946 mmol), and EDC-HCl (0.660 g, 3.44 mmol) were mixed in pyridine (2.3 mL) and stirred for 1 hour at room temperature. The reaction mixture was poured into water, and extracted by EA. The organic layer was dried over anhydrous Na₂SO₄ and concentrated. The residue was purified by prep. HPLC to give compound 186, N-(3-fluorophenyl)-3-((3-phenylcyclobutyl)amino)benzamide (17 mg, 5%) as a white solid.

Synthesis of Compound 187

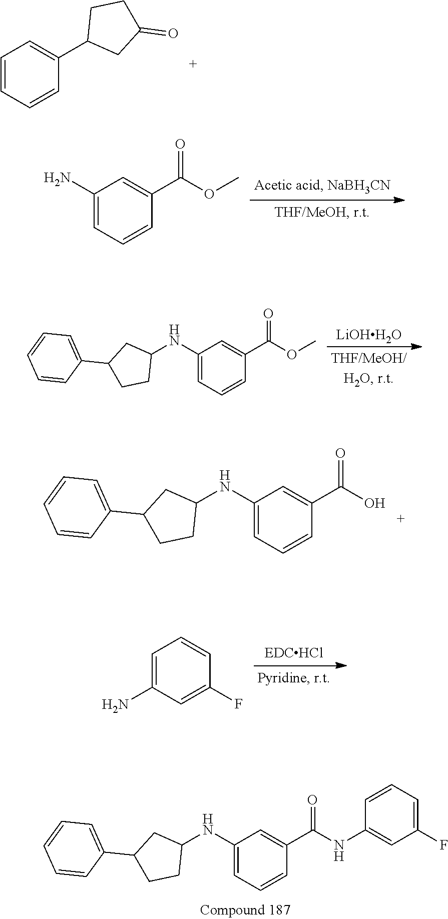

Step 1: 3-Phenylcyclopentan-1-one (0.41 g, 2.58 mmol), methyl 3-aminobenzoate (0.39 g, 2.58 mmol), and acetic acid (1.24 g, 20.64 mmol) were mixed in THF/MeOH (18/9 mL) and heated for 30 minutes at 70° C. The reaction mixture was cooled to room temperature and NaBH₃CN (0.32 g, 5.16 mmol) was added and stirred for 2 hours at room temperature. The reaction mixture was poured into water and extracted by EA. The organic layer was dried over anhydrous Na₂SO₄ and concentrated. The residue was purified by MPLC to give methyl 3-((3-phenylcyclopentyl)amino)benzoate (0.2 g, 26%).

Step 2: Methyl 3-((3-phenylcyclopentyl)amino)benzoate (0.1 g, 0.338 mmol) and LiOH.H₂O (0.056 g, 1.35 mmol) were mixed in THF/MeOH/H₂O (0.33/0.33/0.33 mL) and stirred for 3 hours at room temperature. The reaction mixture acidified by adding 1 N HCl, precipitates were filtered and washed with water to give 3-((3-phenylcyclopentyl)amino)benzoic acid (0.089 g, 93%).

Step 3: 3-((3-Phenylcyclopentyl)amino)benzoic acid (0.075 g, 0.266 mmol), 3-flouroaniline (0.033 g, 0.293 mmol), and 1-ethyl-3-(3-dimethylaminopropyl)carbodiimide hydrochloride (EDC-HCl) (0.2 g, 1.06 mmol) were mixed in pyridine (0.5 mL) and stirred for 1 hour at room temperature. The reaction mixture was poured into water and extracted by EA. The organic layer was dried over anhydrous Na₂SO₄ and concentrated. The residue was purified by prep. HPLC to give compound 187, N-(3-fluorophenyl)-3-((3-phenylcyclopentyl)amino)benzamide (17 mg, 17%) as a white solid.

Synthesis of Compound 188

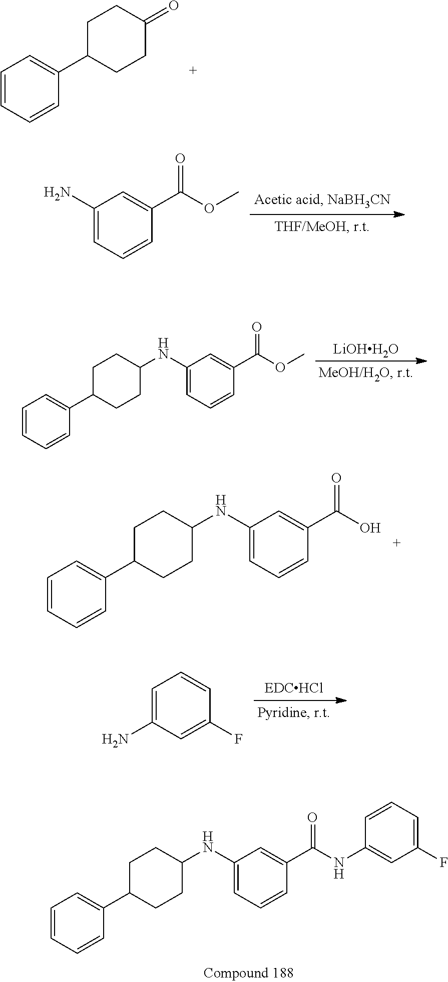

Step 1: 4-Phenylcyclohexan-1-one (0.60 g, 3.44 mmol), methyl 3-aminobenzoate (0.52 g, 3.44 mmol), and acetic acid (1.65 g, 27.55 mmol) were mixed in THF/MeOH (12/6 mL) and heated for 30 minutes at 70° C. The reaction mixture was cooled to room temperature and NaBH₃CN (0.43 g, 6.89 mmol) was added and stirred for 1 hour at room temperature. The reaction mixture was poured into water, and extracted by EA. The organic layer was dried over anhydrous Na₂SO₄ and concentrated. The residue was purified by MPLC to give methyl 3-((4-phenylcyclohexyl)amino)benzoate (0.44 g, 41%).

Step 2: Methyl 3-((4-phenylcyclohexyl)amino)benzoate (0.44 g, 1.42 mmol) and LiOH.H₂O (0.26 g, 5.68 mmol) were mixed in MeOH/H₂O (2.5/2.5 mL) and stirred for 4 hours at room temperature. The reaction mixture acidified by adding 1 N HCl, precipitates were filtered and washed with water to give 3-((4-phenylcyclohexyl)amino)benzoic acid (0.16 g, 38%).

Step 3: 3-((4-Phenylcyclohexyl)amino)benzoic acid (0.26 g, 0.880 mmol), 3-flouroaniline (0.108 g, 0.968 mmol), and EDC-HCl (0.675 g, 3.521 mmol) were mixed in pyridine (5.2 mL) and stirred for 1.5 hours at room temperature. The reaction mixture was poured into water and extracted by EA. The organic layer was dried over anhydrous Na₂SO₄ and concentrated. The residue was purified by prep. HPLC to give compound 188, N-(3-fluorophenyl)-3-((4-phenylcyclohexyl)amino)benzamide (Diastereomer A: 18 mg, 5% as a yellow solid/Diastereomer B: 12 mg, 3.5% as a brown solid)

Synthesis of Compound 189

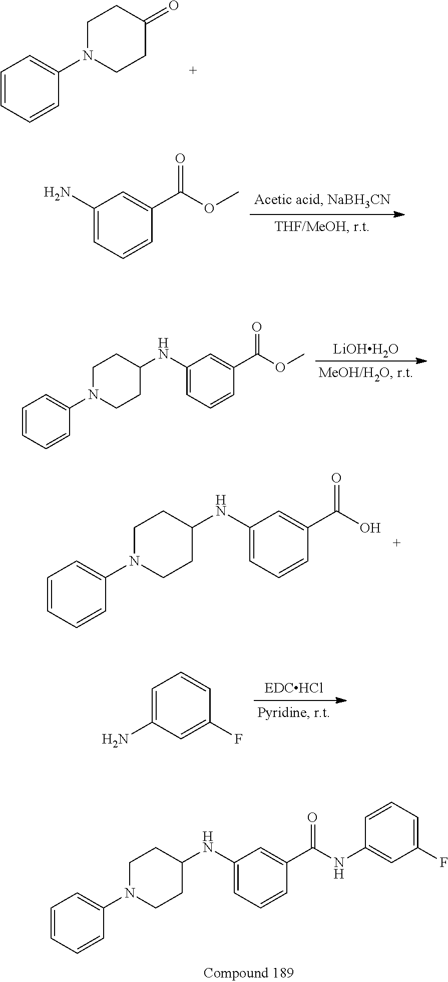

Step 1: 1-Phenylpiperidin-4-one (0.60 g, 3.42 mmol), methyl 3-aminobenzoate (0.41 g, 2.74 mmol), and acetic acid (1.64 g, 27.39 mmol) were mixed in THF/MeOH (20/10 mL) and heated for 30 minutes at 50° C. The reaction mixture was cooled to room temperature and NaBH₃CN (0.43 g, 6.85 mmol) was added and stirred for 2 hours at room temperature. The reaction mixture was poured into water, and extracted by EA. The organic layer was dried over anhydrous Na₂SO₄ and concentrated. The residue was purified by MPLC to give methyl methyl 3-((1-phenylpiperidin-4-yl)amino)benzoate (0.54 g, 50%).

Step 2: Methyl 3-((1-phenylpiperidin-4-yl)amino)benzoate (0.53 g, 1.72 mmol) and LiOH.H₂O (0.29 g, 6.88 mmol) were mixed in THF/MeOH/H₂O (2.5/1.25/1.25 mL) and stirred for 4 hours at room temperature. The reaction mixture acidified by adding 1 N HCl, precipitates were filtered and washed with water to give 3-((1-phenylpiperidin-4-yl)amino)benzoic acid (0.27 g, 53%).

Step 3: 3-((1-Phenylpiperidin-4-yl)amino)benzoic acid (0.36 g, 1.215 mmol), 3-flouroaniline (0.13 g, 1.215 mmol), and EDC-HCl (0.93 g, 4.859 mmol) were mixed in pyridine (3.6 mL) and stirred for 1 hour at room temperature. The reaction mixture was poured into water and extracted by EA. The organic layer was dried over anhydrous Na₂SO₄ and concentrated. The residue was purified by prep. HPLC to give compound 189, N-(3-fluorophenyl)-3-((1-phenylpiperidin-4-yl)amino)benzamide (0.016 g, 3%) as a white solid.

Synthesis of Compound 190

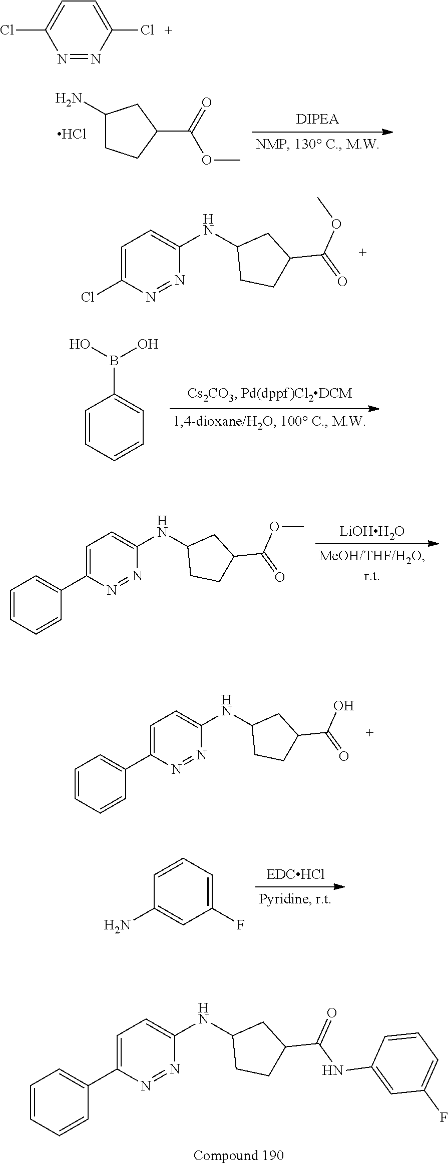

Step 1: 3,6-Dichloropyridazine (0.550 g, 3.692 mmol) and methyl 3-aminocyclopentane-1-carboxylate-HCl (0.730 g, 4.061 mmol) was dissolved in N-Methyl-2-Pyrrolidone (NMP) (5.5 mL), followed up by addition of DIPEA (3.2 mL, 18.460 mmol) and heated in a microwave reactor for 2 hours at 130° C. The reaction mixture was poured into water, and extracted by EA. The organic layer was dried over anhydrous Na₂SO₄ and concentrated. The residue was purified by MPLC to give 3-((6-chloropyridazin-3-yl)amino)cyclopentane-1-carboxylate (0.37 g, 39%).

Step 2: Methyl 3-((6-chloropyridazin-3-yl)amino)cyclopentane-1-carboxylate (0.590 g, 2.31 mmol), phenyl boronic acid (0.338 g, 2.77 mmol), cesium carbonate (1.88 g, 5.77 mmol) and 1,1′-bis(diphenylphosphino)ferrocene]dichloropalladium(II).DCM complex (0.188 g, 0.231 mmol) were mixed in 1,4-dioxane/H₂O (5.9 mL/1.2 mL) and heated in a microwave reactor for 2 hours at 100° C. The reaction mixture was poured into water, and extracted by EA. The organic layer was dried over anhydrous Na₂SO₄ and concentrated. The residue was purified by MPLC to give methyl 3-((6-phenylpyridazin-3-yl)amino)cyclopentane-1-carboxylate (0.15 g, 22%).

Step 3: Methyl 3-((6-phenylpyridazin-3-yl)amino)cyclopentane-1-carboxylate (0.15 g, 0.504 mmol) and LiOH.H₂O (0.086 g, 2.01 mmol) were mixed in THF/MeOH/H₂O (0.75/0.375/0.375 mL) and stirred for 4 hours at room temperature. The reaction mixture acidified by adding 1 N HCl, precipitates were filtered and washed with water to give 3-((6-phenylpyridazin-3-yl)amino)cyclopentane-1-carboxylic acid (0.08 g, 56%).

Step 4: 3-((6-Phenylpyridazin-3-yl)amino)cyclopentane-1-carboxylic acid (0.08 g, 0.282 mmol), 3-flouroaniline (0.034 g, 0.311 mmol), and EDC-HCl (0.217 g, 1.129 mmol) were mixed in pyridine (0.8 mL) and stirred for 1 hour at room temperature. The reaction mixture was poured into water, and extracted by EA. The organic layer was dried over anhydrous Na₂SO₄ and concentrated. The residue was purified by prep. HPLC to give compound 190, N-(3-fluorophenyl)-3-((6-phenylpyridazin-3-yl)amino)cyclopentane-1-carboxamide (13 mg, 12%) as a white solid.

Synthesis of Compound 191

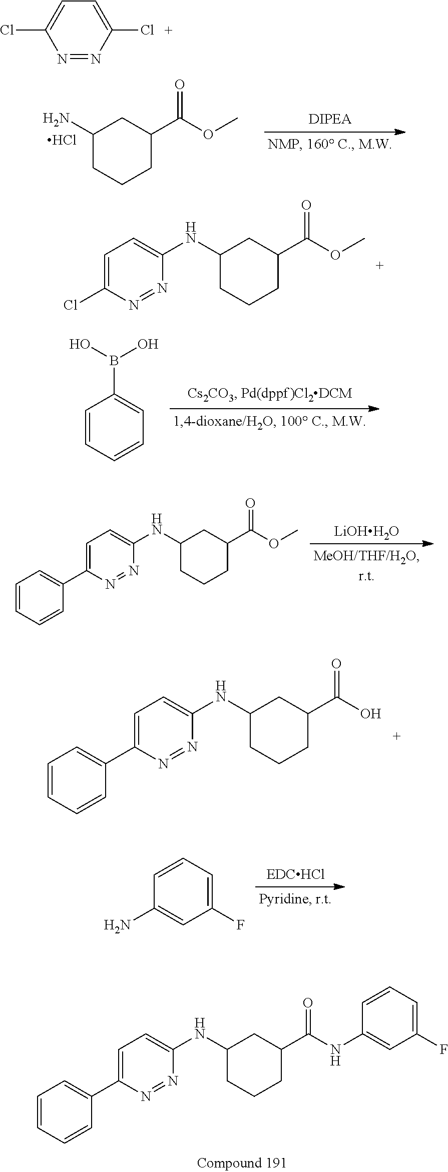

Step 1: 3,6-Dichloropyridazine (0.550 g, 3.692 mmol) and methyl 3-aminocyclohexane-1-carboxylate-HCl (0.786 g, 4.061 mmol) was dissolved in NMP (5.5 mL), followed up by addition of DIPEA (3.2 mL, 18.460 mmol) and heated in a microwave reactor for 3 hours at 160° C. The reaction mixture was poured into water, and extracted by EA. The organic layer was dried over anhydrous Na₂SO₄ and concentrated. The residue was purified by MPLC to give methyl 3-((6-chloropyridazin-3-yl)amino)cyclohexane-1-carboxylate. (0.32 g, 32%).

Step 2: Methyl 3-((6-chloropyridazin-3-yl)amino)cyclohexane-1-carboxylate (0.50 g, 1.854 mmol), phenyl boronic acid (0.271 g, 2.224 mmol), cesium carbonate (1.51 g, 4.634 mmol) and 1,1′-bis(diphenylphosphino)ferrocene]dichloropalladium(II).DCM complex (0.151 mg, 0.185 mmol) were mixed in 1,4-dioxane/H₂O (5.4 mL/0.3 mL) and heated in a microwave reactor for 2 hours at 100° C. The reaction mixture was poured into water, and extracted by EA. The organic layer was dried over anhydrous Na₂SO₄ and concentrated. The residue was purified by MPLC to give methyl 3-((6-phenylpyridazin-3-yl)amino)cyclohexane-1-carboxylate (0.12 g, 21%).

Step 3: Methyl 3-((6-phenylpyridazin-3-yl)amino)cyclohexane-1-carboxylate (0.12 g, 0.385 mmol) and LiOH.H₂O (0.065 g, 1.54 mmol) were mixed in THF/MeOH/H₂O (0.5/0.25/0.25 mL) and stirred for 3 hours at room temperature. The reaction mixture acidified by adding 1 N HCl, precipitates were filtered and washed with water to give 3-((6-phenylpyridazin-3-yl)amino)cyclohexane-1-carboxylic acid (0.06 g, 52%).

Step 4: 3-((6-Phenylpyridazin-3-yl)amino)cyclohexane-1-carboxylic acid (0.225 g, 0.757 mmol), 3-flouroaniline (0.084 g, 0.757 mmol), and EDC-HCl (0.580 g, 3.027 mmol) were mixed in pyridine (4.5 mL) and stirred for 1 hour at room temperature. The reaction mixture was poured into water, and extracted by EA. The organic layer was dried over anhydrous Na₂SO₄ and concentrated. The residue was purified by prep. HPLC to give compound 191, N-(3-fluorophenyl)-3-((6-phenylpyridazin-3-yl)amino)cyclohexane-1-carboxamide (11 mg, 4%) as a white solid.

Table 1 lists the chemical structures, characterization data, and preparation methods of the above-described compounds.

TABLE 1
Compound Structure, Characterization Data, and Preparation Method

| | | | LCMS | |
|---|---|---|---|---|
| Cmpd | Chemical Structure | Characterization Data | [m/z] | Method |
| 1 | 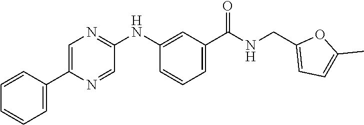 [SMILES: Cc1ccc(CNC(=O)c2cccc(Nc3cnc(-c4ccccc4)cn3)c2)o1] | ¹H NMR (400 MHz, CDCl₃) δ 8.63 (d, J = 1.4 Hz, 1H), 8.34 (d, J = 1.4 Hz, 1H), 8.00 (s, 1H), 7.96-7.93 (m, 2H), 7.80-7.75 (m, 1H), 7.52-7.41 (m, 5H), 6.83 (s, 1H), 6.42 (s, 1H), 6.22 (d, J = 3.0 Hz, 1H), 5.96-5.94 (m, 1H), 4.63 (d, J = 5.4 Hz, 2H), | 385 | Method A |
| | | 2.31 (s, 3H). | | |
| 2 | 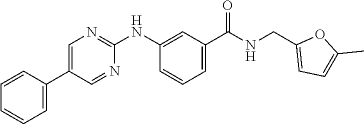 [SMILES: Cc1ccc(CNC(=O)c2cccc(Nc3ncc(-c4ccccc4)cn3)c2)o1] | ¹H NMR (400 MHz, DMSO-d₆) δ 9.91 (s, 1H), 8.90-8.73 (m, 3H), 8.27 (s, 1H), 7.95 (d, J = 7.9 Hz, 1H), 7.78-7.70 (m, 2H), 7.55-7.43 (m, 3H), 7.38 (dd, J = 7.9, 7.9 Hz, 2H), 6.15 (d, J = 2.9 Hz, 1H), 6.00 (d, J = 2.0 Hz, 1H), 4.41 (d, J = 5.6 Hz, 2H), 2.24 (s, 3H). | 385 | Method A |
| 3 | 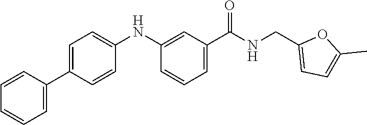 [SMILES: Cc1ccc(CNC(=O)c2cccc(Nc3ccc(-c4ccccc4)cc3)c2)o1] | ¹H NMR (400 MHz, CDCl₃) δ 7.63- 7.51 (m, 5H), 7.49-7.40 (m, 2H), 7.36-7.31 (m, 2H), 7.28-7.23 (m, 2H), 7.21-7.16 (m, 2H), 6.34 (s, 1H), 6.19 (d, J = 3.0 Hz, 1H), 5.94- 5.92 (m, 1H), 5.90 (s, 1H), 4.60 (d, J = 5.3 Hz, 2H), 2.30 (s, 3H). | 383 | Method A |
| 4 | 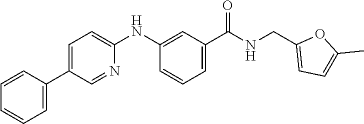 [SMILES: Cc1ccc(CNC(=O)c2cccc(Nc3ccc(-c4ccccc4)cn3)c2)o1] | ¹H NMR (400 MHz, CDCl₃) δ 8.51 (d, J = 2.4 Hz, 1H), 7.89 (s, 1H), 7.79 (dd, J = 2.4, 8.6 Hz, 1H), 7.65-7.61 (m, 1H), 7.56 (d, J = 7.3 Hz, 2H), 7.52-7.34 (m, 5H), 6.96 (d, J = 8.5 Hz, 1H), 6.68 (s, 1H), 6.40 (s, 1H), 6.21 (d, J = 3.0 Hz, 1H), 5.94 (d, J = 2.8 Hz, 1H), 4.62 (d, J = 5.4 Hz, 2H), 2.31 (s, | 384 | Method A |
| | | 3H). | | |
| 5 | 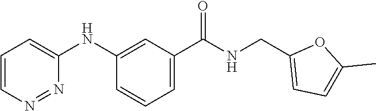 [SMILES: Cc1ccc(CNC(=O)c2cccc(Nc3cccnn3)c2)o1] | ¹H NMR (400 MHz, DMSO-d₆) δ 9.40 (s, 1H), 8.86 (t, J = 5.7 Hz, 1H), 8.69 (dd, J = 1.3, 4.5 Hz, 1H), 8.15 (t, J = 1.8 Hz, 1H), 8.00-7.98 (m, 1H), 7.48-7.37 (m, 3H), 7.14 (dd, J = 1.3, 9.0 Hz, 1H), 6.14 (d, J = 3.0 Hz, 1H), 6.01-5.99 (m, 1H), 4.41 (d, J = 5.6 | 309 | Method A |
| | | Hz, 2H), 2.24 (s, 3H). | | |
| 6 | 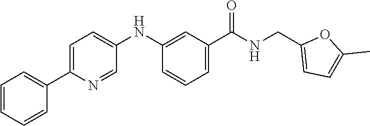 [SMILES: Cc1ccc(CNC(=O)c2cccc(Nc3ccc(-c4ccccc4)nc3)c2)o1] | ¹H NMR (400 MHz, DMSO-d₆) δ 8.91 (t, J = 5.6 Hz, 1H), 8.68 (s, 1H), 8.48 (d, J = 2.6 Hz, 1H), 8.01 (d, J = 7.3 Hz, 2H), 7.88-7.85 (m, 1H), 7.64 (s, 1H), 7.59 (dd, J = 2.8, 8.7 Hz, 1H), 7.46 (t, J = 7.6 Hz, 2H), 7.42- 7.33 (m, 3H), 7.27 (d, J = 7.5 Hz, 1H), 6.13 (d, J = 2.9 Hz, 1H), 5.99 (d, | 384 | Method A |
| | | J = 2.0 Hz, 1H), 4.40 (d, J = 5.5 Hz, | | |
| | | 2H), 2.23 (s, 3H). | | |
| 7 | 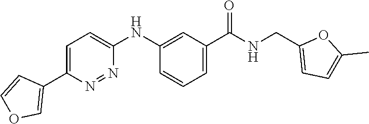 [SMILES: Cc1ccc(CNC(=O)c2cccc(Nc3ccc(-c4ccoc4)nn3)c2)o1] | ¹H NMR (400 MHz, CDCl₃) δ 8.03 (s, 1H), 7.87 (s, 1H), 7.77-7.73 (m, 1H), 7.55 (s, 1H), 7.50-7.43 (m, 3H), 7.15-7.11 (m, 1H), 7.05-7.01 (m, 2H), 6.47-6.46 (m, 1H), 6.22- 6.19 (m, 1H), 5.95-5.93 (m, 1H), 4.61 (d, J = 5.4 Hz, 2H), 2.31-2.30 (m, 3H). | 375 | Method A |
| 8 | 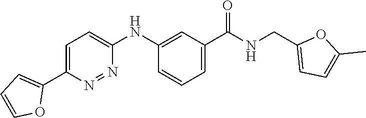 [SMILES: Cc1ccc(CNC(=O)c2cccc(Nc3ccc(-c4ccco4)nn3)c2)o1] | ¹H NMR (400 MHz, DMSO-d₆) δ 9.58 (s, 1H), 8.89 (t, J = 5.6 Hz, 1H), 8.20 (s, 1H), 8.01-7.97(m, 1H), 7.87 (d, J = 1.0 Hz, 1H), 7.85-7.81 (m, 1H), 7.49-7.45 (m, 1H), 7.41 (t, J = 7.8 Hz, 1H), 7.24-7.20 (m, 1H), 7.14 (d, J = 3.3 Hz, 1H), 6.69 (dd, J = 1.8. 3.4 Hz, 1H), 6.15 (d, J = 2.9 | 375 | Method A |
| | | Hz, 1H), 6.00 (d, J = 2.0 Hz, 1H), 4.41 | | |
| | | (d, J = 5.6 Hz, 2H), 2.25 (s, 3H). | | |
| 9 | 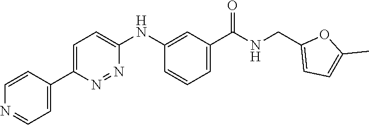 [SMILES: Cc1ccc(CNC(=O)c2cccc(Nc3ccc(-c4ccncc4)nn3)c2)o1] | ¹H NMR (400 MHz, DMSO-d₆) δ 9.74 (s, 1H), 8.95-8.90 (m, 1H), 8.72 (d, J = 6.0 Hz, 2H), 8.25-8.15 (m, 2H), 8.08-8.01 (m, 3H), 7.53- 7.49 (m, 1H), 7.44 (t, J = 7.9 Hz, 1H), 7.30-7.26 (m, 1H), 6.15 (d, J = 2.9 Hz, 1H), 6.02-5.99 (m, 1H), 4.44- 4.40 (m, 2H), 2.24 (s, 3H). | 386 | Method A |
| 10 | 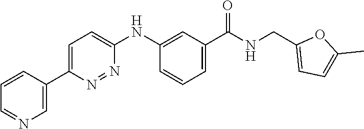 [SMILES: Cc1ccc(CNC(=O)c2cccc(Nc3ccc(-c4cccnc4)nn3)c2)o1] | ¹H NMR (400 MHz, DMSO-d₆) δ 9.65 (s, 1H), 9.25 (d, J = 2.0 Hz, 1H), 8.92 (t, J = 5.6 Hz, 1H), 8.66 (d, J = 3.8 Hz, 1H), 8.47-8.42 (m, 1H), 8.23 (s, 1H), 8.15-8.12 (m, 1H), 8.05-8.01 (m, 1H), 7.58-7.47 (m, 2H), 7.44 (t, J = 7.8 Hz, 1H), 7.29-7.25 (m, 1H), 6.15 (d, J = 3.0 Hz, 1H), 6.00 (d, | 386 | Method A |
| | | J = 2.0 Hz, 1H), 4.42 (d, J = 5.5 Hz, | | |
| | | 2H), 2.24 (s, 3H). | | |
| 11 | 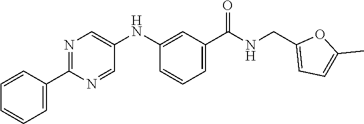 [SMILES: Cc1ccc(CNC(=O)c2cccc(Nc3cnc(-c4ccccc4)nc3)c2)o1] | ¹H NMR (400 MHz, DMSO-d₆) δ 8.96 (t, J = 5.6 Hz, 1H), 8.84 (s, 1H), 8.73 (s, 2H), 8.31 (d, J = 7.0 Hz, 2H), 7.66 (s, 1H), 7.58-7.35 (m, 5H), 7.31 (d, J = 8.0 Hz, 1H), 6.14 (d, J = 2.9 Hz, 1H), 5.99 (d, J = 2.1 Hz, 1H), 4.40 (d, J = 5.6 Hz, 2H), 2.23 (s, 3H). | 385 | Method A |
| 12 | 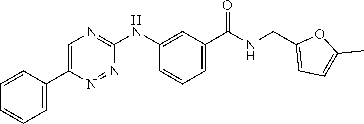 [SMILES: Cc1ccc(CNC(=O)c2cccc(Nc3ncc(-c4ccccc4)nn3)c2)o1] | ¹H NMR (400 MHz, DMSO-d₆) δ 10.41 (s, 1H), 9.11 (s, 1H), 8.93 (t, J = 5.7 Hz, 1H), 8.31 (dd, J = 2.0, 2.0 Hz, 1H), 8.11 (d, J = 7.1 Hz, 2H). 7.93 (dd, J = 1.6, 7.9 Hz, 1H), 7.63-7.47 (m, 4H), 7.44 (t, J = 7.9 Hz, 1H), 6.15 (d, J = 3.0 Hz, 1H), 6.00 (d, J = 2.0 Hz, 1H), 4.41 (d, J = 5.5 Hz, 2H), 2.24 (s, | 386 | Method A |
| | | 3H). | | |
| 13 | 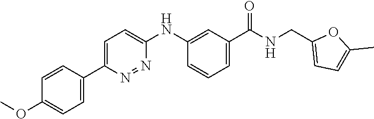 | ¹H NMR (400 MHz, DMSO-d₆) δ 9.49 (s, 1H), 8.89 (t, J = 5.6 Hz, 1H), 8.21 (s, 1H), 8.06-7.95 (m, 4H), 7.52-7.36 (m, 2H), 7.21 (d, J = 9.4 Hz, 1H), 7.08 (d, J = 8.9 Hz, 2H), 6.15 (d, J = 2.9 Hz, 1H), 6.00 (d, J = 2.6 Hz, 1H), 4.42 (d, J = 5.6 Hz, 2H), 3.83 (s, | 415 | Method A |
| | | 3H), 2.25 (s, 3H). | | |
| 15 | 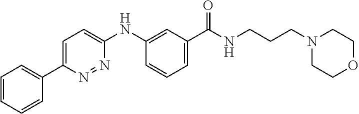 [SMILES: O=C(NCCCN1CCOCC1)c1cccc(Nc2ccc(-c3ccccc3)nn2)c1] | ¹H NMR (400 MHz, DMSO-d₆) δ 9.55 (s, 1H), 8.47 (t, J = 5.4 Hz, 1H), 8.22 (s, 1H), 8.08-7.98 (m, 4H), 7.53 (t, J = 7.4 Hz, 2H), 7.49-7.40 (m, 3H), 7.24 (d, J = 9.4 Hz, 1H), 3.57 (t, J = 4.5 Hz, 4H), 2.39-2.32 (m, 6H), 1.74-1.66 (m, 2H). | 418 | Method A |
| 16 | 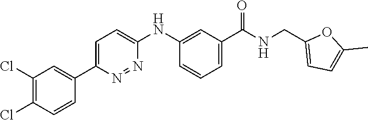 [SMILES: Cc1ccc(CNC(=O)c2cccc(Nc3ccc(-c4ccc(Cl)c(Cl)c4)nn3)c2)o1] | ¹H NMR (400 MHz, DMSO-d₆) δ 9.66 (s, 1H), 8.91 (t, J = 5.5 Hz, 1H), 8.34 (d, J = 1.6 Hz, 1H), 8.24-8.12 (m, 2H), 8.09 (dd, J = 1.4, 8.6 Hz, 1H), 8.03-7.99 (m, 1H), 7.81-7.77 (m, 1H), 7.51-7.47 (m, 1H), 7.43 (t, J = 7.8 Hz, 1H), 7.27-7.23 (m, 1H), | 453 | Method A |
| | | 6.15 (d, J = 2.8 Hz, 1H), 6.01 (s, 1H), | | |
| | | 4.42 (d, J = 5.5 Hz, 2H), 2.25-2.24 | | |
| | | (m, 3H). | | |
| 17 | 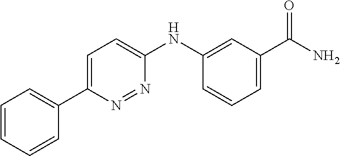 [SMILES: NC(=O)c1cccc(Nc2ccc(-c3ccccc3)nn2)c1] | ¹H NMR (400 MHz, DMSO-d₆) δ 11.25 (s, 1H), 8.45 (d, J = 9.4 Hz, 1H), 8.29 (d, J = 9.4 Hz, 1H), 8.17-8.11 (m, 2H), 7.60-7.52 (m, 3H), 7.26- 7.14 (m, 3H), 6.82-6.79 (m, 1H), 5.45-5.26 (m, 2H). | 291 | Method C |
| 18 | 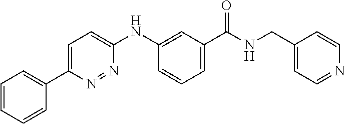 [SMILES: O=C(NCc1ccncc1)c1cccc(Nc2ccc(-c3ccccc3)nn2)c1] | ¹H NMR (400 MHz, DMSO-d₆) δ 9.59 (s, 1H), 9.12 (t, J= 5.9 Hz, 1H), 8.52 (d, J = 5.9 Hz, 2H), 8.27 (s, 1H), 8.15-7.92 (m, 4H), 7.60-7.42 (m, 5H), 7.38-7.29 (m, 2H), 7.25 (d, J = 9.4 Hz, 1H), 4.52 (d, J = 5.9 Hz, 2H). | 382 | Method A |
| 19 | 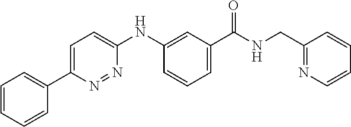 [SMILES: O=C(NCc1ccccn1)c1cccc(Nc2ccc(-c3ccccc3)nn2)c1] | ¹H NMR (400 MHz, DMSO-d₆) δ 9.60 (s, 1H), 9.11 (t, J = 5.9 Hz, 1H), 8.53 (d, J = 4.3 Hz, 1H), 8.27 (dd, J = 2.0, 2.0 Hz, 1H), 8.22-8.01 (m, 4H), 7.81-7.75 (m, 1H), 7.62-7.49 (m, 3H), 7.49-7.41 (m, 2H). 7.39- 7.22 (m, 3H), 4.59 (d, J = 6.0 Hz, 2H). | 382 | Method A |
| 20 | 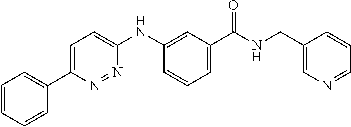 [SMILES: O=C(NCc1cccnc1)c1cccc(Nc2ccc(-c3ccccc3)nn2)c1] | ¹H NMR (400 MHz, DMSO-d₆) δ 9.59 (s, 1H), 9.11 (t, J = 5.9 Hz, 1H), 8.58 (d, J = 1.6 Hz, 1H), 8.47 (dd, J = 1.5, 4.8 Hz, 1H), 8.25 (dd, J = 1.9, 1.9 Hz, 1H), 8.16-8.01 (m, 4H), 7.77-7.73 (m, 1H), 7.57-7.41 (m, 5H), 7.38 (dd, J = 4.8, 7.8 Hz, 1H), 7.25 (d, J = 9.4 Hz, 1H), 4.52 (d, | 382 | Method A |
| | | J = 5.9 Hz, 2H). | | |
| 21 | 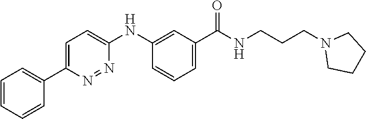 [SMILES: O=C(NCCCN1CCCC1)c1cccc(Nc2ccc(-c3ccccc3)nn2)c1] | ¹H NMR (400 MHz, DMSO-d₆) δ 9.61-9.52 (m, 1H), 8.55 (dd, J = 5.3, 5.3 Hz, 1H), 8.21 (s, 1H), 8.14-7.93 (m, 4H), 7.61-7.32 (m, 5H), 7.24 (d, J = 9.4 Hz, 1H), 3.33-3.29 (m, 2H), 2.48-2.40 (m, 6H), 1.84-1.52 (m, 6H). | 402 | Method A |
| 22 | 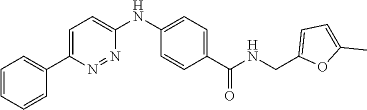 [SMILES: Cc1ccc(CNC(=O)c2ccc(Nc3ccc(-c4ccccc4)nn3)cc2)o1] | ¹H NMR (400 MHz, DMSO-d₆) δ 9.70 (s, 1H), 8.77 (t, J = 5.1 Hz, 1H), 8.10-8.04 (m, 3H), 7.89 (s, 4H), 7.56-7.43 (m, 3H), 7.31-7.26 (m, 1H), 6.13 (s, 1H), 5.98 (s, 1H), 4.42- 4.37 (m, 2H), 2.25-2.22 (m, 3H). | 385 | Method A |
| 23 | 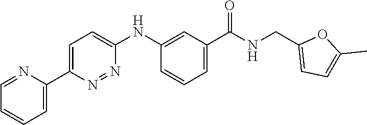 [SMILES: Cc1ccc(CNC(=O)c2cccc(Nc3ccc(-c4ccccn4)nn3)c2)o1] | ¹H NMR (400 MHz, DMSO-d₆) δ 9.71 (s, 1H), 8.93 (t, J = 5.6 Hz, 1H), 8.70 (d, J = 4.5 Hz, 1H), 8.46 (d, J = 8.0 Hz, 1H), 8.35 (d, J = 9.4 Hz, 1H), 8.26 (s, 1H), 8.03 (d, J = 7.9 Hz, 1H), 8.00-7.94 (m, 1H), 7.58-7.38 (m, 3H), 7.29 (d, J = 9.4 Hz, 1H), 6.15 (d, J = 2.9 Hz, 1H), 6.00 (d, J = 2.0 Hz, | 386 | Method A |
| | | 1H), 4.42 (d, J = 5.5 Hz, 2H), 2.24 (s, | | |
| | | 3H). | | |
| 24 | 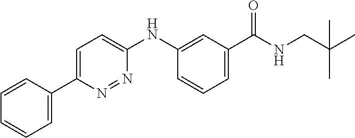 [SMILES: CC(C)(C)CNC(=O)c1cccc(Nc2ccc(-c3ccccc3)nn2)c1] | ¹H NMR (400 MHz, DMSO-d₆) δ 9.56 (s, 1H), 8.34 (t, J = 6.3 Hz, 1H), 8.15 (s, 1H), 8.09-8.03 (m, 4H), 7.52 (t, J = 7.4 Hz, 2H), 7.48-7.42 (m, 3H), 7.25 (d, J = 9.4 Hz, 1H), 3.12 (d, J = 6.4 Hz, 2H), 0.92 (s, 9H). | 361 | Method A |
| 25 | 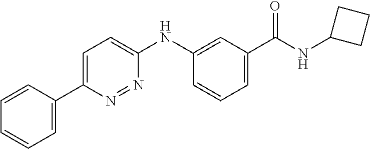 [SMILES: O=C(NC1CCC1)c1cccc(Nc2ccc(-c3ccccc3)nn2)c1] | ¹H NMR (400 MHz, DMSO-d₆) δ 9.56 (s, 1H), 8.62 (d, J = 7.8 Hz, 1H), 8.15 (d, J = 1.8 Hz, 1H), 8.11-7.96 (m, 4H), 7.52 (dd, J = 7.4, 7.4 Hz, 2H), 7.49-7.37 (m, 3H), 7.24 (d, J = 9.4 Hz, 1H), 4.48-4.40 (m, 1H), 2.27-2.19 (m, 2H), 2.15-2.02 (m, 2H), 1.73-1.62 (m, 2H). | 345 | Method A |
| 26 | 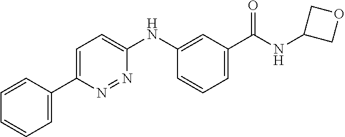 [SMILES: O=C(NC1COC1)c1cccc(Nc2ccc(-c3ccccc3)nn2)c1] | ¹H NMR (400 MHz, DMSO-d₆) δ 9.60 (s, 1H), 9.12 (d, J = 6.4 Hz, 1H), 8.21 (s, 1H), 8.09-8.04 (m, 4H), 7.55-7.42 (m, 5H), 7.26-7.23 (m, 1H), 5.07-4.98 (m, 1H), 4.78 (t, J = 6.9 Hz, 2H), 4.61 (t, J = 6.4 Hz, 2H). | 347 | Method A |
| 27 | 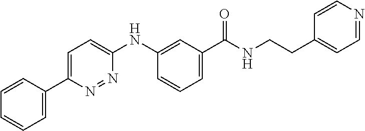 [SMILES: O=C(NCCc1ccncc1)c1cccc(Nc2ccc(-c3ccccc3)nn2)c1] | ¹H NMR (400 MHz, DMSO-d₆) δ 9.58 (s, 1H), 8.58 (t, J = 5.4 Hz, 1H), 8.49 (d, J = 5.8 Hz, 2H), 8.21 (s, 1H), 8.13-8.03 (m, 3H), 7.99 (d, J = 7.9 Hz, 1H), 7.53 (dd, J = 7.4, 7.4 Hz, 2H), 7.49-7.35 (m, 3H), 7.30 (d, J = 5.8 Hz, 2H), 7.24 (d, J = 9.4 Hz, 1H), 3.55 (q, J = 6.6 Hz, 2H), 2.90 (t, | 396 | Method A |
| | | J = 7.1 Hz, 2H). | | |
| 28 | 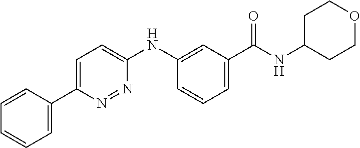 [SMILES: O=C(NC1CCOCC1)c1cccc(Nc2ccc(-c3ccccc3)nn2)c1] | ¹H NMR (400 MHz, DMSO-d₆) δ 9.58 (s, 1H), 8.34 (d, J = 7.8 Hz, 1H), 8.14 (d, J = 1.8 Hz, 1H), 8.12-8.00 (m, 4H), 7.53 (dd, J = 7.4, 7.4 Hz, 2H), 7.49-7.35 (m, 3H), 7.24 (d, J = 9.4 Hz, 1H), 4.08-3.97 (m, 1H), 3.89 (dd, J = 2.1, 11.7 Hz, 2H), 3.49- 3.31 (m, 2H), 1.77 (dd, J = 2.4, 12.5 H, 2H), 1.59 (ddd, J = 4.2, 12.1, 24.0 | 375 | Method A |
| | | Hz, 2H). | | |
| 29 | 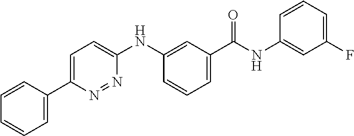 [SMILES: O=C(Nc1cccc(F)c1)c1cccc(Nc2ccc(-c3ccccc3)nn2)c1] | ¹H NMR (400 MHz, DMSO-d₆) δ 10.49 (s, 1H), 9.67 (s, 1H), 8.32 (dd, J = 2.0, 2.0 Hz, 1H), 8.21-7.95 (m, 4H), 7.81-7.75 (m, 1H), 7.65-7.35 (m, 7H), 7.27 (d, J = 9.4 Hz, 1H), 6.98-6.91 (m, 1H). | 385 | Method A |
| 30 | 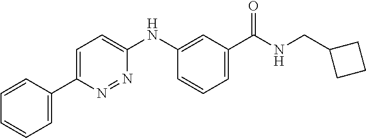 [SMILES: O=C(NCC1CCC1)c1cccc(Nc2ccc(-c3ccccc3)nn2)c1] | ¹H NMR (400 MHz, DMSO-d₆) δ 9.57 (s, 1H), 8.44 (t, J = 5.8 Hz, 1H), 8.16 (s, 1H), 8.11-7.94 (m, 4H), 7.58-7.49 (m, 2H), 7.49-7.35 (m, 3H), 7.24 (d, J = 9.3 Hz, 1H), 3.33- 3.30 (m, 2H), 2.59-2.53 (m, 1H, 2.08-1.91 (m, 2H), 1.89-1.63 (m, 4H). | 359 | Method A |
| 31 | 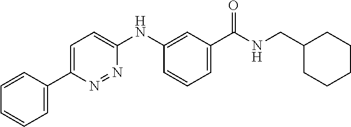 [SMILES: O=C(NCC1CCCCC1)c1cccc(Nc2ccc(-c3ccccc3)nn2)c1] | ¹H NMR (400 MHz, DMSO-d₆) δ 9.55 (s, 1H), 8.41 (t, J = 5.7 Hz, 1H), 8.15 (d, J = 1.8 Hz, 1H), 8.11-7.99 (m, 4H), 7.53 (dd, J = 7.4, 7.4 Hz, 2H), 7.49-7.37 (m, 3H), 7.24 (d, J = 9.4 Hz, 1H), 3.12 (dd, J = 6.4, 6.4 Hz, 2H), 1.89-1.39 (m, 6H), 1.38- 1.03 (m, 4H), 0.98-0.85 (m, 1H). | 387 | Method A |
| 32 | 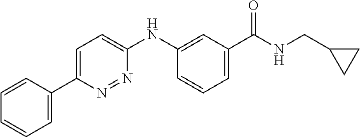 [SMILES: O=C(NCC1CC1)c1cccc(Nc2ccc(-c3ccccc3)nn2)c1] | ¹H NMR (400 MHz, DMSOd₆) δ 9.57 (s, 1H), 8.56 (t, J = 5.6 Hz, 1H), 8.20 (s, 1H), 8.12-7.93 (m, 4H), 7.53 (dd, J = 7.4, 7.4 Hz, 2H), 7.49- 7.38 (m, 3H), 7.25 (d, J = 9.3 Hz, 1H), 3.16 (dd, J = 6.2, 6.2 Hz, 2H), 1.09- 1.00 (m, 1H), 0.47-0.41 (m, 2H), 0.27-0.22 (m, 2H). | 345 | Method A |
| 33 | 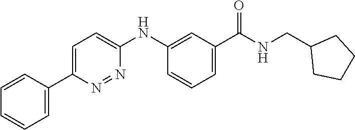 [SMILES: O=C(NCC1CCCC1)c1cccc(Nc2ccc(-c3ccccc3)nn2)c1] | ¹H NMR (400 MHz, DMSO-d₆) δ 9.56 (s, 1H), 8.46 (t, J = 5.7 Hz, 1H), 8.16 (s, 1H), 8.13-7.97 (m, 4H), 7.52 (dd, J = 7.4, 7.4 Hz, 2H), 7.49- 7.37 (m, 3H), 7.25 (d, J = 9.4 Hz, 1H), 3.20 (dd, J = 5.9, 7.1 Hz, 2H), 2.23- 2.11 (m, 1H), 1.80-1.37 (m, 6H), 1.32- 1.22 (m, 2H). | 373 | Method A |
| 34 | 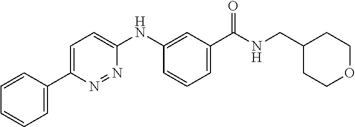 [SMILES: O=C(NCC1CCOCC1)c1cccc(Nc2ccc(-c3ccccc3)nn2)c1] | ¹H NMR (400 MHz, DMSO-d₆) δ 9.56 (s, 1H), 8.48 (t, J =5.8 Hz, 1H), 8.17 (d, J = 1.8 Hz, 1H), 8.11-7.91 (m, 4H), 7.52 (dd, J = 7.4. 7.4 Hz, 2H), 7.49-7.36 (m, 3H), 7.24 (d, J = 9.4 Hz, 1H), 3.86 (dd, J = 2.6, 11.4 Hz, 2H), 3.32-3.23 (m, 2H), 3.17 (dd, J = 6.3, 6.3 Hz, 2H), 1.87-1.76 | 389 | Method A |
| | | (m, 1H), 1.65-1.59 (m, 2H), 1.27- | | |
| | | 1.15 (m, 2H). | | |
| 35 | 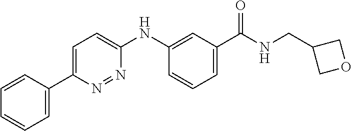 [SMILES: O=C(NCC1COC1)c1cccc(Nc2ccc(-c3ccccc3)nn2)c1] | ¹H NMR (400 MHz, DMSO-d₆) δ 9.57 (s, 1H), 8.61 (t, J = 5.6 Hz, 1H), 8.19 (d, J = 1.1 Hz, 1H), 8.11-7.98 (m, 4H), 7.58-7.49 (m, 2H), 7.49- 7.35 (m, 3H), 7.24 (d, J = 9.4 Hz, 1H), 4.65 (dd, J = 6.0, 7.8 Hz, 2H), 4.37 (dd, J = 6.0, 6.0 Hz, 2H), 3.56 (dd, J = 6.4, 6.4 Hz, 2H), 3.24-3.12 (m, | 361 | Method A |
| | | 1H). | | |
| 36 | 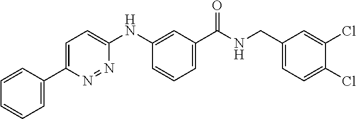 [SMILES: O=C(NCc1ccc(Cl)c(Cl)c1)c1cccc(Nc2ccc(-c3ccccc3)nn2)c1] | ¹H NMR (400 MHz, DMSCO-d₆) δ 9.59 (s, 1H), 9.11 (t, J = 6.0 Hz, 1H), 8.26 (s, 1H), 8.11-8.01 (m, 4H), 7.69-7.56 (m, 2H), 7.56-7.40 (m, 5H), 7.34 (dd, J = 2.1. 8.3 Hz, 1H), 7.25 (d, J = 9.4 Hz, 1H), 4.48 (d, J = 5.9 Hz, 2H). | 449 | Method A |
| 37 | 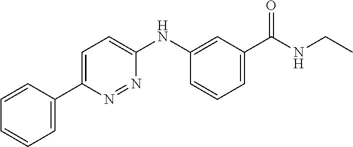 [SMILES: CCNC(=O)c1cccc(Nc2ccc(-c3ccccc3)nn2)c1] | ¹H NMR (400 MHz, DMSO-d₆) δ 9.57 (s, 1H), 8.47 (t, J = 5.4 Hz, 1H), 8.19 (s, 1H), 8.13-7.94 (m, 4H), 7.53 (dd, J = 7.4, 7.4 Hz, 2H), 7.49- 7.35 (m, 3H), 7.24 (d, J = 9.3 Hz, 1H), 3.33-3.25 (m, 2H), 1.14 (t, J = 7.2 Hz, 3H). | 319 | Method A |
| 38 | 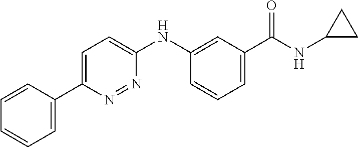 [SMILES: O=C(NC1CC1)c1cccc(Nc2ccc(-c3ccccc3)nn2)c1] | ¹H NMR (400 MHz, DMSO-d₆) δ 9.57 (s, 1H), 8.44 (d, J = 4.1 Hz, 1H), 8.15 (d, J = 1.0 Hz, 1H), 8.11-7.96 (m, 4H), 7.52 (dd, J = 7.3, 7.3 Hz, 2H), 7.49-7.35 (m, 3H), 7.24 (d, J = 9.4 Hz, 1H), 2.86 (ddd, J = 3.7, 7.6, 14.9 Hz, 1H), 0.73-0.67 (m, 2H), 0.61-0.55 (m, 2H). | 331 | Method A |
| 39 | 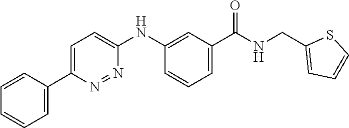 [SMILES: O=C(NCc1cccs1)c1cccc(Nc2ccc(-c3ccccc3)nn2)c1] | ¹H NMR (400 MHz, DMSO-d₆) δ 9.59 (s, 1H), 9.14 (t, J = 5.9 Hz, 1H), 8.24 (s, 1H), 8.12-7.94 (m, 4H), 7.53 (dd, J = 7.4, 7.4 Hz, 2H), 7.49- 7.36 (m, 4H), 7.25 (d, J = 9.3 Hz, 1H), 7.04 (d, J = 3.3 Hz, 1H), 6.98 (dd, J = 3.5, 5.0 Hz, 1H), 4.64 (d, J = 5.8 Hz, 2H). | 387 | Method A |
| 40 | 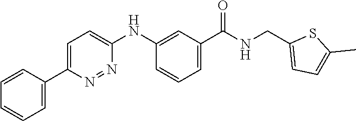 [SMILES: Cc1ccc(CNC(=O)c2cccc(Nc3ccc(-c4ccccc4)nn3)c2)s1] | ¹H NMR (400 MHz, DMSO-d₆) δ 9.58 (s, 1H), 9.08 (t, J = 5.9 Hz, 1H), 8.23 (s, 1H), 8.12-7.92 (m, 4H), 7.52 (dd, J = 7.3, 7.3 Hz, 2H), 7.49- 7.37 (m, 3H), 7.24 (d, J = 9.4 Hz, 1H), 6.80 (d, J = 3.3 Hz, 1H), 6.63 (dd, J = 1.1, 3.3 Hz, 1H). 4.54 (d, J = 5.8 Hz, 2H), 2.39 (s, 3H). | 401 | Method A |
| 41 | 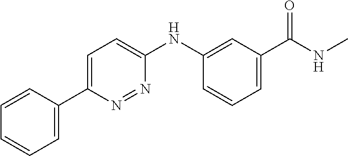 [SMILES: CNC(=O)c1cccc(Nc2ccc(-c3ccccc3)nn2)c1] | ¹H NMR (400 MHz, DMSO-d₆) δ 9.57 (s, 1H), 8.45-8.39 (m, 1H), 8.23-8.21 (m, 1H), 8.10-8.03 (m, 3H), 8.02-7.98 (m, 1H), 7.53 (t, J = 7.4 Hz, 2H), 7.49-7.40 (m, 3H), 7.27-7.23 (m, 1H), 2.81-2.78 (m, 3H). | 305 | Method A |
| 42 | 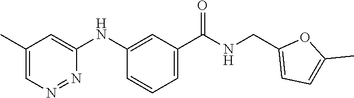 | ¹H NMR (400 MHz, DMSO-d₆) δ 9.31 (s, 1H), 8.88 (t, J = 5.7 Hz, 1H), 8.58 (d, J = 1.5 Hz, 1H), 8.13 (s, 1H), 8.00-7.98 (m, 1H), 7.45-7.36 (m, 2H), 6.93 (s, 1H), 6.14 (d, J = 3.0 Hz, 1H), 6.00 (d, J = 3.0 Hz, 1H), 4.40 (d, | 323 | Method A |
| | | J = 5.6 Hz, 2H), 2.25 (d, J = 5.5 Hz, | | |
| | | 6H). | | |
| 43 | 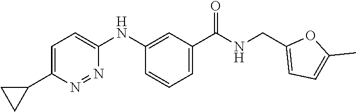 [SMILES: Cc1ccc(CNC(=O)c2cccc(Nc3ccc(C4CC4)nn3)c2)o1] | ¹H NMR (400 MHz, DMSO-d₆) δ 9.27 (s, 1H), 8.89-8.83 (m, 1H), 8.15 (s, 1H), 7.96-7.93 (m, 1H), 7.42-7.34 (m, 2H), 7.31-7.28 (m, 1H), 7.06-7.03 (m, 1H), 6.14 (d, J = 3.0 Hz, 1H), 6.00 (s, 1H), 4.42- 4.38 (m, 2H), 2.24 (s, 3H), 2.16- | 349 | Method A |
| | | 2.08 (m, 1H), 1.02-0.90 (m, 4H). | | |
| 44 | 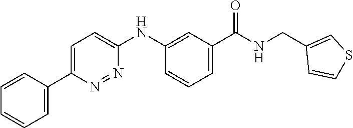 [SMILES: O=C(NCc1ccsc1)c1cccc(Nc2ccc(-c3ccccc3)nn2)c1] | ¹H NMR (400 MHz, DMSO-d₆) δ 9.58- 9.56 (m, 1H), 8.98 (t, J = 5.9 Hz, 1H), 8.24-8.23 (m, 1H), 8.10- 8.03 (m, 4H), 7.55-7.40 (m, 6H), 7.34 (d, J = 1.8 Hz, 1H), 7.27-7.23 (m, 1H), 7.13-7.10 (m, 1H), 4.50- 4.46 (m, 2H). | 387 | Method A |
| 45 | 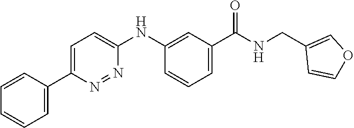 [SMILES: O=C(NCc1ccoc1)c1cccc(Nc2ccc(-c3ccccc3)nn2)c1] | ¹H NMR (400 MHz, DMSO-d₆) δ 9.57 (s, 1H), 8.85 (t, J = 5.8 Hz, 1H), 8.22 (s, 1H), 8.09-8.02 (m, 4H), 7.61 (d, J = 1.5 Hz, 2H), 7.55-7.50 (m, 2H), 7.48-7.40 (m, 3H), 7.25 (d, J = 9.4 Hz, 1H), 6.49 (s, 1H), 4.32 (d, J = 5.9 Hz, 2H). | 371 | Method A |
| 46 | 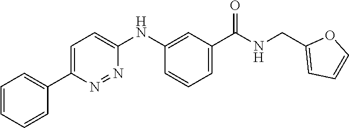 [SMILES: O=C(NCc1ccco1)c1cccc(Nc2ccc(-c3ccccc3)nn2)c1] | ¹H NMR (400 MHz, DMSO-d₆) δ 9.57 (s, 1H), 8.96 (t, J = 5.8 Hz, 1H), 8.22 (s, 1H), 8.15-7.95 (m, 4H), 7.60 (d, J = 1.0 Hz, 1H), 7.53 (dd, J = 7.4, 7.4 Hz, 2H), 7.49-7.38 (m, 3H), 7.25 (d, J = 9.4 Hz, 1H), 6.41 (dd, J = 1.9, 3.1 Hz. 1H), 6.29 (d, J = 2.9 Hz, 1H), 4.48 (d, J = 5.6 Hz, | 371 | Method A |
| | | 2H). | | |
| 47 | 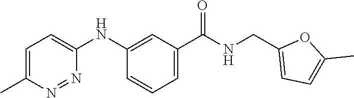 | ¹H NMR (400 MHz, DMSO-d₆) δ 9.29 (s, 1H), 8.87 (t, J = 5.7 Hz, 1H), 8.12 (s, 1H), 7.97 (d, J = 7.8 Hz, 1H), 7.47-7.31 (m, 3H), 7.08 (d, J = 9.0 Hz, 1H), 6.14 (d, J = 3.0 Hz, 1H), 5.99 (d, J = 1.9 Hz, 1H), 4.40 (d, J = 5.6 Hz, | 323 | Method A |
| | | 2H), 2.48 (s, 3H), 2.24 (s, 3H). | | |
| 48 | 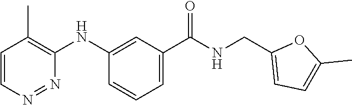 [SMILES: Cc1ccc(CNC(=O)c2cccc(Nc3nnccc3C)c2)o1] | ¹H NMR (400 MHz, DMSO-d₆) δ 8.85 (t, J = 5.6 Hz, 1H), 8.59 (d, J = 4.6 Hz, 1H), 8.26 (s, 1H), 8.15 (s, 1H), 7.96 (dd, J = 1.4, 7.9 Hz, 1H), 7.48 (d, J = 7.8 Hz, 1H), 7.43-7.29 (m, 2H), 6.14 (d, J = 2.9 Hz, 1H), 5.99 (d, J = 2.0 Hz, 1H), 4.40 (d, J = 5.6 Hz, | 323 | Method A |
| | | 2H), 2.31 (s, 3H), 2.24 (s, 3H). | | |
| 49 | 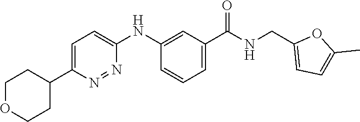 [SMILES: Cc1ccc(CNC(=O)c2cccc(Nc3ccc(C4CCOCC4)nn3)c2)o1] | ¹H NMR (400 MHz, DMSO-d₆) δ 9.33 (s, 1H), 8.87 (t, J = 5.6 Hz, 1H), 8.16 (s, 1H), 7.97 (d, J = 7.8 Hz, 1H), 7.48-7.33 (m, 3H), 7.11 (d, J = 9.1 Hz, 1H), 6.14 (d, J = 3.0 Hz, 1H), 5.99 (d, J = 2.0 Hz, 1H), 4.40 (d, J = 5.6 Hz, 2H), 4.00-3.94 (m, 2H), 3.51-3.43 (m, 2H), 3.06-2.96 (m, 1H), 2.24 (s, | 393 | Method A |
| | | 3H), 1.82-1.74 (m, 4H). | | |
| 50 | 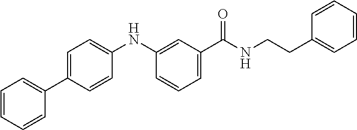 [SMILES: O=C(NCCc1ccccc1)c1cccc(Nc2ccc(-c3ccccc3)cc2)c1] | ¹H NMR (400 MHz, DMSO-d₆) δ 8.53-8.47 (m, 2H), 7.68-7.54 (m, 5H), 7.44 (dd, J = 7.8, 7.8 Hz, 2H), 7.34-7.20 (m, 11H), 3.51-3.44 (m, 2H), 2.84 (t, J = 7.4 Hz, 2H). | 393 | Method A |
| 51 | 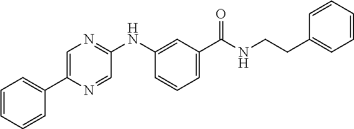 [SMILES: O=C(NCCc1ccccc1)c1cccc(Nc2cnc(-c3ccccc3)cn2)c1] | ¹H NMR (400 MHz, DMSO-d₆) δ 9.81 (s, 1H), 8.78 (d,J = 1.3 Hz, 1H), 8.54 (t, J = 5.6 Hz, 1H), 8.35 (d, J = 1.3 Hz, 1H), 8.15 (s, 1H), 8.02 (d, J = 7.3 Hz, 2H), 7.95-7.90 (m, 1H), 7.54- 7.35 (m, 5H), 7.35-7.15 (m, 5H), 3.53-3.46 (m, 2H), 2.86 (t, J = 7.4 Hz, 2H). | 395 | Method A |
| 52 | 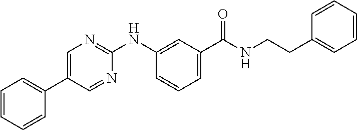 [SMILES: O=C(NCCc1ccccc1)c1cccc(Nc2ncc(-c3ccccc3)cn2)c1] | ¹H NMR (400 MHz, DMSO-d₆) δ 9.93 (s, 1H), 8.87 (s, 2H), 8.50 (t, J = 5.6 Hz, 1H), 8.24 (s, 1H), 7.95- 7.91 (m, 1H), 7.76-7.72 (m, 2H), 7.49 (dd, J = 7.6, 7.6 Hz, 2H), 7.44- 7.16 (m, 8H), 3.53-3.45 (m, 2H), 2.86 (t, J = 7.4 Hz, 2H). | 395 | Method A |
| 53 | 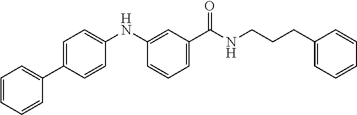 [SMILES: O=C(NCCCc1ccccc1)c1cccc(Nc2ccc(-c3ccccc3)cc2)c1] | ¹H NMR (400 MHz, DMSO-d₆) δ 8.48 (s, 1H), 8.44 (t, J = 5.5 Hz, 1H), 7.68-7.54 (m, 5H), 7.43 (dd, J = 7.8, 7.8 Hz, 2H), 7.36-7.26 (m, 5H), 7.26-7.21 (m, 3H), 7.21-7.12 (m, 3H), 3.27 (q, J = 6.6 Hz, 2H), 2.63 (t, J = 7.6 Hz, 2H), 1.88-1.78 (m, 2H). | 407 | Method A |
| 54 | 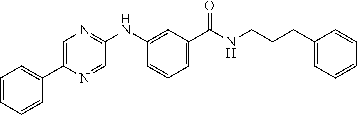 [SMILES: O=C(NCCCc1ccccc1)c1cccc(Nc2cnc(-c3ccccc3)cn2)c1] | ¹H NMR (400 MHz, DMSO-d₆) δ 9.81 (s, 1H), 8.78 (d, J = 1.3 Hz, 1H), 8.47 (t, J = 5.5 Hz, 1H), 8.34 (d, J = 1.4 Hz, 1H), 8.14 (d, J = 1.9 Hz, 1H), 8.06-7.85 (m, 3H), 7.53-7.33 (m, 5H), 7.33-7.22 (m, 4H), 7.19 (t, J = 7.1 Hz, 1H), 3.32-3.27 (m, 2H), 2.65 | 409 | Method A |
| | | (dd, J = 7.7, 7.7 Hz, 2H), 1.89-1.79 | | |
| | | (m, 2H). | | |
| 55 | 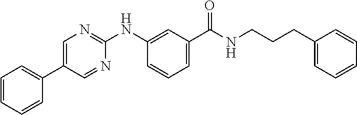 [SMILES: O=C(NCCCc1ccccc1)c1cccc(Nc2ncc(-c3ccccc3)cn2)c1] | ¹H NMR (400 MHz, DMSO-d₆) δ 9.92 (s, 1H), 8.86 (s, 2H), 8.43 (t, J = 5.6 Hz, 1H), 8.24 (s, 1H), 7.95 (d, J = 7.6 Hz, 1H), 7.73 (d, J = 7.3 Hz, 2H), 7.49 (dd, J = 7.6, 7.6 Hz, 2H), 7.45-7.34 (m, 3H), 7.34-7.22 (m, 4H), 7.19 (t, J = 7.1 Hz, 1H), 3.32- | 409 | Method A |
| | | 3.25 (m, 2H), 2.65 (dd, J = 7.6, 7.6 | | |
| | | Hz, 2H), 1.89-1.79 (m, 2H). | | |
| 57 | 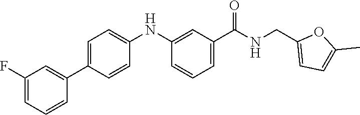 [SMILES: Cc1ccc(CNC(=O)c2cccc(Nc3ccc(-c4cccc(F)c4)cc3)c2)o1] | ¹H NMR (400 MHz, DMSO-d₆) δ 8.90 (t, J = 5.6 Hz, 1H), 8.56 (s, 1H), 7.78-7.59 (m, 3H), 7.56-7.40 (m, 3H), 7.40-7.29 (m, 2H), 7.27-7.23 (m, 1H), 7.21-7.02 (m, 3H), 6.13 (d, J = 2.9 Hz, 1H), 5.99 (d, J = 1.9 Hz, 1H), 4.39 (d, J = 5.6 Hz, 2H), 2.23 (s, | 401 | Method A |
| | | 3H). | | |
| 58 | 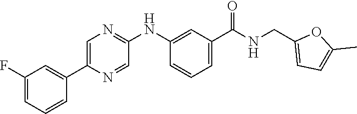 [SMILES: Cc1ccc(CNC(=O)c2cccc(Nc3cnc(-c4cccc(F)c4)cn3)c2)o1] | ¹H NMR (400 MHz, DMSO-d₆) δ 9.90 (s, 1H), 8.92 (t, J = 5.7 Hz, 1H), 8.85 (d, J = 1.3 Hz, 1H), 8.34 (d, J = 1.3 Hz, 1H), 8.18 (s, 1H), 7.95 (dd, J = 1.3. 8.0 Hz, 1H), 7.92-7.76 (m, 2H), 7.57-7.36 (m, 3H), 7.24-7.18 (m, 1H), 6.15 (d, J = 3.0 Hz, 1H), 6.00 | 403 | Method A |
| | | (d, J = 2.0 Hz, 1H), 4.41 (d, J = 5.6 Hz, | | |
| | | 2H), 2.24 (s, 3H). | | |
| 60 | 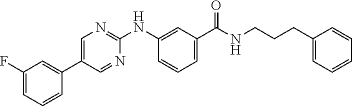 [SMILES: O=C(NCCCc1ccccc1)c1cccc(Nc2ncc(-c3cccc(F)c3)cn2)c1] | ¹H NMR (400 MHz, DMSO-d₆) δ 9.99 (s, 1H), 8.91 (s, 2H), 8.44 (t, J = 5.5 Hz, 1H), 8.24 (s, 1H), 7.93 (dd, J = 1.9,6.0 Hz, 1H), 7.72-7.57 (m, 2H), 7.56-7.49 (m, 1H), 7.47-7.34 (m, 2H), 7.33-7.13 (m, 6H), 3.29 (q, J = 6.6 Hz, 2H), 2.65 (dd, J = 7.7, 7.7 | 427 | Method A |
| | | Hz, 2H), 1.89-1.80 (m, 2H). | | |
| 61 | 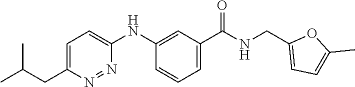 [SMILES: Cc1ccc(CNC(=O)c2cccc(Nc3ccc(CC(C)C)nn3)c2)o1] | ¹H NMR (400 MHz, DMSO-d₆) δ 9.29 (s, 1H), 8.85 (t, J = 5.7 Hz, 1H), 8.15 (s, 1H), 7.98 (d, J = 7.8 Hz, 1H), 7.47-7.31 (m, 3H), 7.09 (d, J = 9.1 Hz, 1H), 6.14 (d, J = 2.9 Hz, 1H), 5.99 (d, J = 2.0 Hz, 1H), 4.41 (d, J = 5.6 Hz, | 365 | Method A |
| | | 2H), 2.65 (d, J = 7.3 Hz, 2H), 2.24 (s, | | |
| | | 3H), 2.08-1.97 (m, 1H), 0.91 (d, | | |
| | | J = 6.6 Hz, 6H). | | |
| 62 | 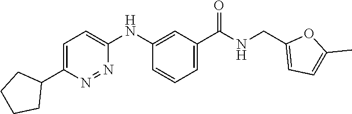 [SMILES: Cc1ccc(CNC(=O)c2cccc(Nc3ccc(C4CCCC4)nn3)c2)o1] | ¹H NMR (400 MHz, DMSO-d₆) δ 9.28 (s, 1H), 8.85 (t, J = 5.7 Hz, 1H), 8.16 (dd, J = 1.9, 1.9 Hz, 1H), 7.99- 7.95 (m, 1H), 7.45-7.33 (m, 3H), 7.09 (d, J = 9.3 Hz, 1H), 6.14 (d, J = 3.0 Hz, 1H), 6.01-5.99 (m, 1H), 4.40 (d, J = 5.8 Hz, 2H), 3.27-3.18 | 377 | Method A |
| | | (m, 1H), 2.24 (s, 3H), 2.04-1.98 (m, | | |
| | | 2H), 1.87-1.57 (m, 6H). | | |
| 63 | 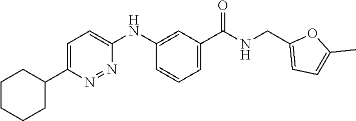 [SMILES: Cc1ccc(CNC(=O)c2cccc(Nc3ccc(C4CCCCC4)nn3)c2)o1] | ¹H NMR (400 MHz, DMSO-d₆) δ 9.28 (s, 1H), 8.85 (t, J = 5.7 Hz, 1H), 8.14 (d, J = 1.6 Hz, 1H), 7.96 (dd, J = 2.2, 7.7 Hz, 1H), 7.45-7.33 (m, 3H), 7.09 (d, J = 9.1 Hz, 1H), 6.14 (d, J = 2.9 Hz, 1H), 5.99 (d, J = 2.0 Hz, 1H), 4.40 (d, J = 5.6 Hz, 2H), 2.79- 2.71 (m, 1H), 2.24 (s, 3H), 1.94- | 391 | Method A |
| | | 1.62 (m, 5H), 1.61-1.15 (m, 5H). | | |
| 65 | 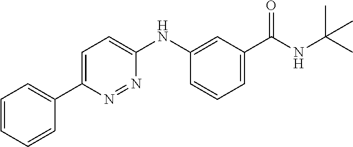 [SMILES: CC(C)(C)NC(=O)c1cccc(Nc2ccc(-c3ccccc3)nn2)c1] | ¹H NMR (400 MHz, DMSO-d₆) δ 9.52 (s, 1H), 8.16-7.96 (m, 5H), 7.74 (s, 1H), 7.53 (dd, J = 7.4, 7.4 Hz, 2H), 7.48-7.44 (m, 1H), 7.43-7.32 (m, 2H), 7.24 (d, J = 9.4 Hz, 1H), 1.40 (s, 9H). | 347 | Method A |
| 66 | 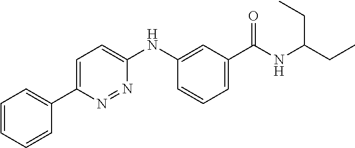 [SMILES: CCC(CC)NC(=O)c1cccc(Nc2ccc(-c3ccccc3)nn2)c1] | ¹H NMR (400 MHz, DMSO-d₆) δ 9.53 (s, 1H), 8.13 (d, J = 1.6 Hz, 1H), 8.10-7.95 (m, 5H), 7.58-7.49 (m, 2H), 7.49-7.38 (m, 3H), 7.25 (d, J = 9.4 Hz, 1H), 3.85-3.75 (m, 1H), 1.65-1.39 (m, 4H), 0.88 (dd, J = 7.4, 7.4 Hz, 6H). | 361 | Method A |
| 67 | 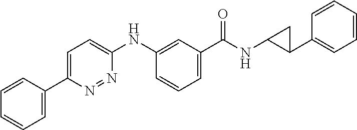 [SMILES: O=C(NC1CC1c1ccccc1)c1cccc(Nc2ccc(-c3ccccc3)nn2)c1] | ¹H NMR (400 MHz, DMSO-d₆) δ 9.58 (s, 1H), 8.70 (d, J = 4.4 Hz, 1H), 8.19-8.18 (m, 1H), 8.11-7.99 (m, 4H), 7.52 (dd, J = 7.4, 7.4 Hz, 2H), 7.49-7.38 (m, 3H), 7.34-7.22 (m, 3H), 7.22-7.13 (m, 3H), 3.09-3.03 (m, 1H), 2.13-2.07 (m, 1H), 1.41- 1.35 (m, 1H), 1.28-1.21 (m, 1H). | 407 | Method B |
| 68 | 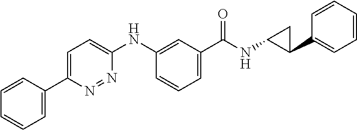 [SMILES: O=C(N[C@@H]1C[C@H]1c1ccccc1)c1cccc(Nc2ccc(-c3ccccc3)nn2)c1] | ¹H NMR (400 MHz, DMSO-d₆) δ 9.58 (s, 1H), 8.70 (d, J = 4.4 Hz, 1H), 8.19 (s, 1H), 8.11-7.97 (m, 4H), 7.52 (dd, J = 7.3, 7.3 Hz, 2H), 7.49- 7.39 (m, 3H), 7.34-7.22 (m, 3H), 7.22-7.07 (m, 3H), 3.10-3.03 (m, 1H), 2.13-2.07 (m, 1H), 1.41-1.35 (m, 1H), 1.28-1.21 (m, 1H). | 407 | Method B |
| 69 | 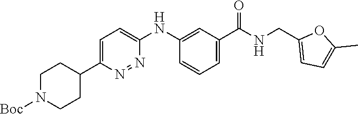 [SMILES: Cc1ccc(CNC(=O)c2cccc(Nc3ccc(C4CCN(C(=O)OC(C)(C)C)CC4)nn3)c2)o1] | ¹H NMR (400 MHz, DMSO-d₆) δ 9.32 (s, 1H), 8.85 (dd, J = 5.7, 5.7 Hz, 1H), 8.15 (d, J = 1.6 Hz, 1H), 7.98- 7.96 (m, 1H), 7.48-7.33 (m, 3H), 7.10 (d, J = 9.1 Hz, 1H), 6.14 (d, J = 3.0 Hz, 1H), 5.99 (d, J = 1.9 Hz, 1H), 4.42-4.39 (m, 2H), 4.12-4.02 | 492 | Method A |
| | | (m, 2H), 3.15-2.70 (m, 3H), 2.24 (s, | | |
| | | 3H), 1.97-1.71 (m, 2H), 1.66-1.54 | | |
| | | (m, 2H), 1.43 (s, 9H). | | |
| 70 | 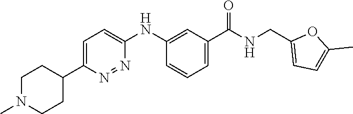 | ¹H NMR (400 MHz, DMSO-d₆) δ 9.32 (s, 1H), 8.85 (t, J = 5.6 Hz, 1H), 8.16 (s, 1H), 7.97 (d, J = 8.0 Hz, 1H), 7.52-7.32 (m, 3H), 7.11 (d, J = 9.1 Hz, 1H), 6.14 (d, J = 2.9 Hz, 1H), 5.99 (d, J = 1.9 Hz, 1H), 4.40 (d, J = 5.6 Hz, 2H), 2.93 (d, J = 10.8 Hz, 2H), 2.76- | 406 | Method A |
| | | 2.67 (m, 1H), 2.29-2.19 (m, 6H), | | |
| | | 2.09 (s, 2H), 1.86-1.76 (m, 4H). | | |
| 71 | 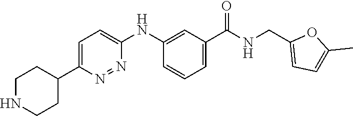 [SMILES: Cc1ccc(CNC(=O)c2cccc(Nc3ccc(C4CCNCC4)nn3)c2)o1] | ¹H NMR (400 MHz, DMSO-d₆) δ 9.30 (s, 1H), 8.85 (t, J = 5.6 Hz, 1H), 8.15 (dd, J = 2.0, 2.0 Hz, 1H), 7.97 (d, J = 8.0 Hz, 1H), 7.51-7.33 (m, 3H), 7.10 (d, J = 9.1 Hz, 1H), 6.14 (d, J = 3.0 Hz, 1H), 5.99 (d, J = 1.9 Hz, 1H), 4.40 (d, J = 5.6 Hz, 2H), 3.06 (d, | 392 | Method A |
| | | J = 12.1 Hz, 2H), 2.88-2.79 (m, 1H), | | |
| | | 2.67-2.58 (m, 2H), 2.24 (s, 3H), | | |
| | | 1.78 (d, J = 11.6 Hz, 2H), 1.61 (ddd, | | |
| | | J = 3.7, 12.2, 24.5 Hz, 2H), 1.31-1.16 | | |
| | | (m, 1H). | | |
| 72 | 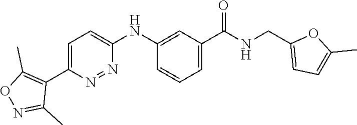 [SMILES: Cc1ccc(CNC(=O)c2cccc(Nc3ccc(-c4c(C)noc4C)nn3)c2)o1] | ¹H NMR (400 MHz, DMSO-d₆) δ 9.55 (s, 1H), 8.88 (t, J = 5.7 Hz, 1H), 8.18 (dd, J = 2.0, 2.0 Hz, 1H), 8.03 (d, J = 7.9 Hz, 1H), 7.67 (d, J = 9.3 Hz, 1H), 7.61-7.37 (m, 2H), 7.24 (d, J = 9.3 Hz, 1H), 6.15 (d, J = 3.0 Hz, 1H), 6.00 (d, J = 2.0 Hz, 1H), 4.42 (d, J = 5.6 Hz, 2H), 2.56 (s, 3H), 2.38- | 404 | Method A |
| | | 2.37 (m, 3H), 2.24 (s, 3H). | | |
| 73 | 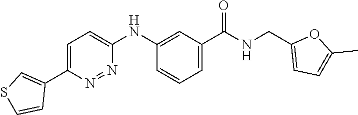 [SMILES: Cc1ccc(CNC(=O)c2cccc(Nc3ccc(-c4ccsc4)nn3)c2)o1] | ¹H NMR (400 MHz, DMSO-d₆) δ 9.50 (s, 1H), 8.88 (t, J = 5.6 Hz, 1H), 8.23 (dd, J = 1.9, 1.9 Hz, 1H), 8.16 (dd, J = 1.3, 2.9 Hz, 1H), 8.06-7.95 (m, 2H), 7.82 (dd, J = 1.3, 5.0 Hz, 1H), 7.69 (dd, J = 2.9, 5.0 Hz, 1H), 7.51-7.37 (m, 2H), 7.20 (d, J = 9.3 | 391 | Method A |
| | | Hz, 1H), 6.15 (d, J = 3.0 Hz, 1H), 6.00 | | |
| | | (s, 1H), 4.42 (d, J = 5.6 Hz, 2H), 2.25 | | |
| | | (s, 3H). | | |
| 74 | 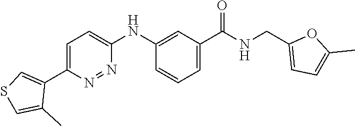 [SMILES: Cc1ccc(CNC(=O)c2cccc(Nc3ccc(-c4cscc4C)nn3)c2)o1] | ¹H NMR (400 MHz, DMSO-d₆) δ 9.49 (s, 1H), 8.88 (t, J = 5.7 Hz, 1H), 8.18 (s, 1H), 8.05-8.03 (m, 1H), 7.86 (d, J = 3.3 Hz, 1H), 7.80-7.76 (m, 1H), 7.48-7.39 (m, 2H), 7.31 (dd, J = 1.1, 3.3 Hz, 1H), 7.23-7.19 (m, 1H), 6.15 (d, J = 3.0 Hz, 1H). 6.00 (d, H = 2.0 Hz, 1H), 4.42 (d, J = | 405 | Method A |
| | | 5.6 Hz, 2H), 2.44 (s, 3H), 2.24 (s, 3H). | | |
| 75 | 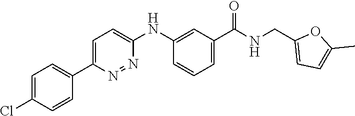 [SMILES: Cc1ccc(CNC(=O)c2cccc(Nc3ccc(-c4ccc(Cl)cc4)nn3)c2)o1] | ¹H NMR (400 MHz, DMSO-d₆) δ 9.60 (s, 1H), 8.90 (t, J = 5.7 Hz, 1H), 8.22 (dd, J = 2.0. 2.0 Hz, 1H), 8.14- 7.95 (m, 4H), 7.58 (d, J = 8.8 Hz, 2H), 7.52-7.38 (m, 2H), 7.25 (d, J = 9.4 Hz, 1H), 6.15 (d, J = 3.0 Hz, 1H), 6.00 (d, J = 1.9 Hz, 1H), 4.42 (d, J = 5.6 Hz, | 419 | Method A |
| | | 2H), 2.24 (s, 3H). | | |
| 76 | 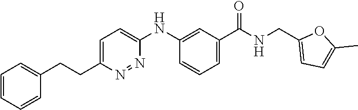 [SMILES: Cc1ccc(CNC(=O)c2cccc(Nc3ccc(CCc4ccccc4)nn3)c2)o1] | ¹H NMR (400 MHz, DMSO-d₆) δ 9.31 (s, 1H), 8.86 (t, J = 5.7 Hz, 1H), 8.15 (s, 1H), 7.98-7.96 (m, 1H), 7.47-7.33 (m, 3H), 7.32-7.13 (m, 5H), 7.07 (d, J = 9.0 Hz, 1H), 6.14 (d, J = 3.0 Hz, 1H), 6.00 (d, J = 2.0 Hz, 1H), 4.40 (d, J = 5.6 Hz, 2H), 3.12- | 413 | Method A |
| | | 3.06 (m, 2H), 3.04-2.97 (m, 2H), | | |
| | | 2.24 (s, 3H). | | |
| 77 | 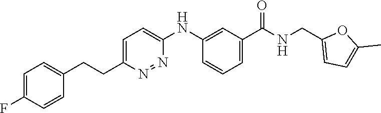 [SMILES: Cc1ccc(CNC(=O)c2cccc(Nc3ccc(CCc4ccc(F)cc4)nn3)c2)o1] | ¹H NMR (400 MHz, DMSO-d₆) δ 9.31 (s, 1H), 8.86 (t, J = 5.7 Hz, 1H), 8.15 (dd, J = 2.0, 2.0 Hz, 1H), 7.98- 7.96 (m, 1H), 7.48-7.32 (m, 3H), 7.32-7.23 (m, 2H), 7.16-7.05 (m, 3H), 6.14 (d, J = 3.0 Hz, 1H), 6.00 (d, J = 2.0 Hz, 1H), 4.41 (d, J = 5.6 Hz, | 431 | Method A |
| | | 2H), 3.11-3.04 (m, 2H), 3.03-2.97 | | |
| | | (m, 2H), 2.24 (s, 3H). | | |
| 78 | 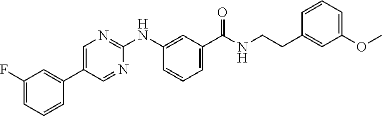 [SMILES: COc1cccc(CCNC(=O)c2cccc(Nc3ncc(-c4cccc(F)c4)cn3)c2)c1] | ¹H NMR (400 MHz, DMSO-d₆) δ 9.99 (s, 1H), 8.91 (s, 2H), 8.48 (dd, J = 5.4, 5.4 Hz, 1H), 8.25 (s, 1H), 7.94- 7.89 (m, 1H), 7.72-7.47 (m, 3H), 7.46-7.33 (m, 2H), 7.31-7.14 (m, 2H), 6.93-6.71 (m, 3H), 3.73 (s, 3H), 3.53-3.45 (m, 2H), 2.84 (t, | 443 | Method A |
| | | J = 7.3 Hz, 2H). | | |
| 79 | 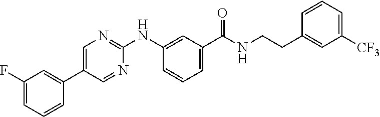 [SMILES: O=C(NCCc1cccc(C(F)(F)F)c1)c1cccc(Nc2ncc(-c3cccc(F)c3)cn2)c1] | ¹H NMR (400 MHz, DMSO-d₆) δ 10.00 (s, 1H), 8.91 (s, 2H), 8.52 (dd, J = 5.5, 5.5 Hz, 1H), 8.23 (s, 1H), 7.94-7.89 (m, 1H), 7.69-7.47 (m, 7H), 7.43-7.28 (m, 2H), 7.23-7.18 (m, 1H), 3.53 (q, J = 6.6 Hz, 2H), 2.97 (t, J = 7.1 Hz, 2H). | 481 | Method B |
| 80 | 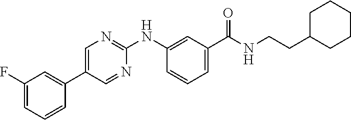 [SMILES: O=C(NCCC1CCCCC1)c1cccc(Nc2ncc(-c3cccc(F)c3)cn2)c1] | ¹H NMR (400 MHz, DMSO-d₆) δ 9.97 (s, 1H), 8.91 (s, 2H), 8.34 (t, J = 5.6 Hz, 1H), 8.22 (s, 1H), 7.94- 7.90 (m, 1H), 7.71-7.56 (m, 2H), 7.56-7.49 (m, 1H), 7.46-7.33 (m, 2H), 7.23-7.17 (m, 1H), 3.31-3.25 (m, 2H), 1.78-1.58 (m, 5H), 1.44 (q, J = 7.0 Hz, 2H), 1.38-1.03 (m, 4H), | 419 | Method A |
| | | 0.96-0.86 (m, 2H). | | |
| 81 | 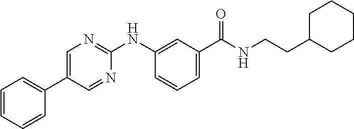 [SMILES: O=C(NCCC1CCCCC1)c1cccc(Nc2ncc(-c3ccccc3)cn2)c1] | ¹H NMR (400 MHz, DMSO-d₆) δ 9.93 (s, 1H), 8.86 (s, 2H), 8.36 (t, J = 5.6 Hz, 1H), 8.22 (d, J = 1.6 Hz, 1H), 7.95-7.91 (m, 1H), 7.86-7.67 (m, 2H), 7.52-7.46 (m, 2H), 7.44- 7.34 (m, 3H), 3.30-3.26 (m, 2H), 1.85-1.54 (m, 5H), 1.43 (q, J = 7.1 Hz, 2H), 1.38-1.06 (m, 4H), 0.97- | 401 | Method B |
| | | 0.85 (m, 2H). | | |
| 82 | 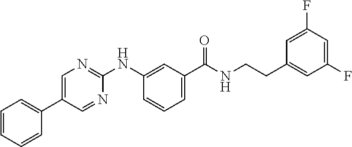 [SMILES: O=C(NCCc1cc(F)cc(F)c1)c1cccc(Nc2ncc(-c3ccccc3)cn2)c1] | ¹H NMR (400 MHz, DMSO-d₆) δ 9.92 (s, 1H), 8.86 (s, 2H), 8.63-8.60 (m, 1H), 8.24 (s, 1H), 7.94-7.89 (m, 1H), 7.75-7.71 (m, 2H), 7.52-7.46 (m, 2H), 7.41-7.29 (m, 4H), 7.10- 7.04 (m, 2H), 3.46 (q, J = 6.6 Hz, 2H), 2.95-2.90 (m, 2H). | 431 | Method A |
| 83 | 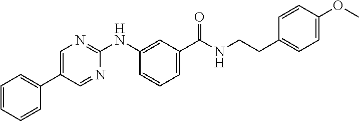 [SMILES: COc1ccc(CCNC(=O)c2cccc(Nc3ncc(-c4ccccc4)cn3)c2)cc1] | ¹H NMR (400 MHz, DMSO-d₆) δ 9.93 (s, 1H), 8.87 (s, 2H), 8.46 (t, J = 5.5 Hz, 1H), 8.24 (s, 1H), 7.94- 7.90 (m, 1H), 7.76-7.72 (m, 2H), 7.49 (t, J = 7.7 Hz, 2H), 7.41-7.36 (m, 3H), 7.20-7.15 (m, 2H), 6.90- 6.85 (m, 2H), 3.72 (s, 3H), 3.44 (dd, J = 6.2, 14.5 Hz, 2H), 2.79 (t, J = 7.4 | 425 | Method A |
| | | Hz, 2H). | | |
| 84 | 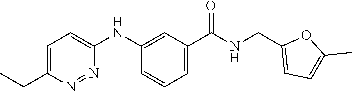 [SMILES: CCc1ccc(Nc2cccc(C(=O)NCc3ccc(C)o3)c2)nn1] | ¹H NMR (400 MHz, DMSO-d₆) δ 9.30 (s, 1H), 8.86 (t, J = 5.6 Hz, 1H), 8.14 (s, 1H), 7.98 (d, J = 7.9 Hz, 1H), 7.48-7.32 (m, 3H), 7.10 (d, J = 9.1 Hz, 1H), 6.14 (d, J = 3.0 Hz, 1H), 6.00 (d, J = 1.9 Hz, 1H), 4.40 (d, J = 5.6 Hz, | 337 | Method A |
| | | 2H), 2.79 (q, J = 7.6 Hz, 2H), 2.24 (s, | | |
| | | 3H), 1.24 (t, J = 7.6 Hz, 3H). | | |
| 85 | 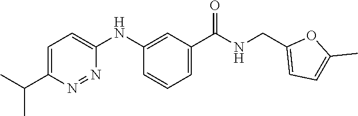 [SMILES: Cc1ccc(CNC(=O)c2cccc(Nc3ccc(C(C)C)nn3)c2)o1] | ¹H NMR (400 MHz, DMSO-d₆) δ 9.30 (s, 1H), 8.86 (t, J = 5.6 Hz, 1H), 8.17 (s, 1H), 7.98-7.96 (m, 1H), 7.53-7.33 (m, 3H), 7.10 (d, J = 9.1 Hz, 1H), 6.14 (d, J = 3.0 Hz, 1H), 6.00 (d, J = 2.0 Hz, 1H), 4.40 (d, J = 5.6 Hz, 2H), 3.16-3.05 (m, 1H), 2.24 (s, | 351 | Method A |
| | | 3H), 1.26 (d, J = 6.9 Hz, 6H). | | |
| 86 | 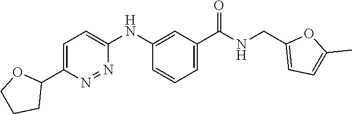 [SMILES: Cc1ccc(CNC(=O)c2cccc(Nc3ccc(C4CCCO4)nn3)c2)o1] | ¹H NMR (400 MHz, DMSO-d₆) δ 9.43 (s, 1H), 8.88 (dd, J = 5.7, 5.7 Hz, 1H), 8.16 (dd, J = 1.9, 1.9Hz, 1H), 7.99-7.96 (m, 1H), 7.50 (d, J = 9.1 Hz, 1H), 7.46-7.35 (m, 2H), 7.16 (d, J = 9.3 Hz, 1H), 6.14 (d, J = 3.0 Hz, 1H), 6.00 (dd, J = 1.0, 3.0 Hz, 1H), | 379 | Method A |
| | | 4.96 (dd, J = 6.8, 6.8 Hz, 1H), 4.42- | | |
| | | 4.39 (m, 2H), 4.00-3.93 (m, 1H), | | |
| | | 3.88-3.81 (m, 1H), 2.33-2.26 (m, | | |
| | | 1H), 2.24 (s, 3H), 2.05-1.85 (m, 3H). | | |
| 87 | 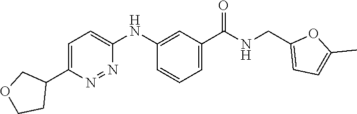 [SMILES: Cc1ccc(CNC(=O)c2cccc(Nc3ccc(C4CCOC4)nn3)c2)o1] | ¹H NMR (400 MHz, DMSO-d₆) δ 9.37 (s, 1H), 8.88 (dd, J = 5.7, 5.7 Hz, 1H), 8.16 (dd, J = 1.9, 1.9 Hz, 1H), 7.98-7.96 (m, 1H), 7.48-7.34 (m, 3H), 7.12 (d, J = 9.1 Hz, 1H), 6.14 (d, J = 2.9 Hz, 1H), 6.01-5.99 (m, 1H), 4.52-4.35 (m, 2H), 4.08 (dd, J = 7.9, | 379 | Method A |
| | | 7.9 Hz, 1H), 3.98-3.71 (m, 3H), | | |
| | | 3.66-3.56 (m, 1H), 2.36-2.26 (m, | | |
| | | 1H), 2.24 (s, 3H), 2.13 (ddd, J = 5.3, | | |
| | | 10.0, 17.6 Hz, 1H). | | |
| 88 | 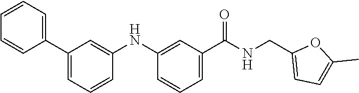 [SMILES: Cc1ccc(CNC(=O)c2cccc(Nc3cccc(-c4ccccc4)c3)c2)o1] | ¹H NMR (400 MHz, DMSO-d₆) δ 8.88 (t, J = 5.7 Hz, 1H), 8.46 (s, 1H), 7.70-7.56 (m, 3H), 7.49-7.44 (m, 2H), 7.41-7.29 (m, 5H), 7.27-7.23 (m, 1H), 7.19-7.05 (m, 2H), 6.11 (d, J = 3.0 Hz, 1H), 5.98 (dd, J = 1.1, 2.9 | 383 | Method A |
| | | Hz, 1H), 4.38 (d, J = 5.6 Hz, 2H), 2.22 | | |
| | | (s, 3H). | | |
| 89 | 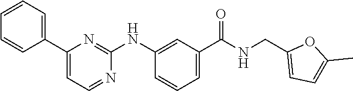 [SMILES: Cc1ccc(CNC(=O)c2cccc(Nc3nccc(-c4ccccc4)n3)c2)o1] | ¹H NMR (400 MHz, DMSO-d₆) δ 9.84 (s, 1H), 8.87 (t, J = 5.7 Hz, 1H), 8.58 (d, J = 5.3 Hz, 1H), 8.43 (dd, J = 2.0, 2.0 Hz, 1H), 8.24-8.20 (m, 2H), 7.93 (dd, J = 1.1. 8.0 Hz, 1H), 7.63-7.49 (m, 3H), 7.49-7.43 (m, | 385 | Method A |
| | | 2H), 7.39 (dd, J = 7.8, 7.8 Hz, 1H), | | |
| | | 6.15 (d, J = 2.9 Hz, 1H), 6.00 (s, 1H), | | |
| | | 4.42 (d, J = 5.6 Hz, 2H), 2.23 (s, 3H). | | |
| 90 | 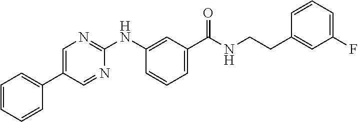 [SMILES: O=C(NCCc1cccc(F)c1)c1cccc(Nc2ncc(-c3ccccc3)cn2)c1] | ¹H NMR (400 MHz, DMSO-d₆) δ 9.93 (s, 1H), 8.86 (s, 2H), 8.50 (t, J = 5.4 Hz, 1H), 8.24 (s, 1H), 7.95- 7.90 (m, 1H), 7.80-7.67 (m, 2H), 7.56-7.45 (m, 2H), 7.45-7.24 (m, 4H), 7.19-6.94 (m, 3H), 3.51 (q, J = 6.6 Hz, 2H), 2.89 (t, J = 7.2 Hz, 2H). | 413 | Method A |
| 92 | 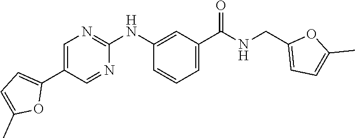 [SMILES: Cc1ccc(CNC(=O)c2cccc(Nc3ncc(-c4ccc(C)o4)cn3)c2)o1] | ¹H NMR (400 MHz, DMSO-d₆) δ 9.95 (s, 1H), 8.85 (t, J = 5.6 Hz, 1H), 8.79 (s, 2H), 8.23 (s, 1H), 7.93-7.88 (m, 1H), 7.45 (d, J = 7.6 Hz, 1H), 7.37 (dd, J = 7.8, 7.8 Hz, 1H), 6.82 (d, J = 3.0 Hz, 1H), 6.22 (d, J = 2.1 Hz, 1H), 6.14 (d, J = 2.6 Hz, 1H), 6.00 (s, 1H), 4.40 (d, J = 5.5 Hz, 2H), 2.37 (s, 3H), 2.25 (s, 3H). | 389 | Method A |
| 93 | 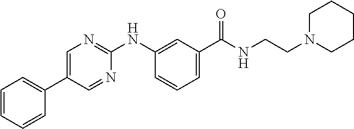 [SMILES: O=C(NCCN1CCCCC1)c1cccc(Nc2ncc(-c3ccccc3)cn2)c1] | ¹H NMR (400 MHz, CDCl₃) δ 8.71 (s, 2H), 8.16 (d, J = 1.1 Hz, 1H), 7.89-7.85 (m, 1H), 7.57-7.41 (m, 7H), 7.37 (s, 1H), 7.07-7.01 (m, 1H), 3.61-3.51 (m, 2H), 2.61 (t, J = 6.0 Hz, 2H), 2.47 (s, 4H), 1.68-1.60 (m, 4H), 1.49 (d, J = 5.3 Hz, 2H). | 402 | Method B |
| 94 | 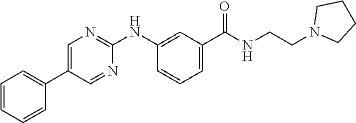 [SMILES: O=C(NCCN1CCCC1)c1cccc(Nc2ncc(-c3ccccc3)cn2)c1] | ¹H NMR (400 MHz, CDCl₃) δ 8.70 (s, 2H), 8.12 (d, J = 1.1 Hz, 1H), 7.92- 7.88 (m, 1H), 7.59-7.47 (m, 4H), 7.47-7.38 (m, 3H), 7.35 (s, 1H), 6.95 (s, 1H), 3.63-3.58 (m, 2H), 2.77 (t, J = 6.0 Hz, 2H), 2.65-2.60 (m, 4H), 1.87-1.82 (m, 4H). | 388 | Method B |
| 95 | 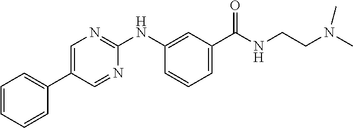 [SMILES: CN(C)CCNC(=O)c1cccc(Nc2ncc(-c3ccccc3)cn2)c1] | ¹H NMR (400 MHz, CDCl₃) δ 8.70 (s, 2H), 8.11 (d, J = 1.1 Hz, 1H), 7.94- 7.90 (m, 1H), 7.63-7.38 (m, 7H), 7.36 (s, 1H), 6.92 (s, 1H), 3.60-3.54 (m, 2H), 2.57 (t, J = 5.9 Hz, 2H), 2.32 (s, 6H). | 362 | Method B |
| 96 | 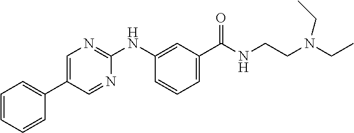 [SMILES: CCN(CC)CCNC(=O)c1cccc(Nc2ncc(-c3ccccc3)cn2)c1] | ¹H NMR (400 MHz, CDCl₃) δ 8.70 (s, 2H), 8.17 (d, J = 1.1 Hz, 1H), 7.88- 7.84 (m, 1H), 7.58-7.38 (m, 7H), 7.35 (s, 1H), 7.07-7.01 (m, 1H), 3.57-3.52 (m, 2H), 2.72 (t, J = 5.9 Hz, 2H), 2.63 (q, J = 7.1 Hz, 4H), 1.10 (t, J = 7.1 Hz, 6H). | 390 | Method B |
| 97 | 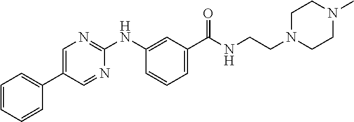 [SMILES: CN1CCN(CCNC(=O)c2cccc(Nc3ncc(-c4ccccc4)cn3)c2)CC1] | ¹H NMR (400 MHz, CDCl₃) δ 8.71 (s, 2H), 8.14 (d, J = 1.6 Hz, 1H), 7.91- 7.87 (m, 1H), 7.59-7.38 (m, 7H), 7.35 (s, 1H), 6.88 (s, 1H), 3.59 (q, J = 5.6 Hz, 2H), 2.68-2.52 (m, 9H), 2.32 (s, 3H). | 417 | Method B |
| 98 | 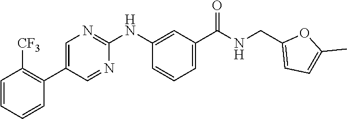 [SMILES: Cc1ccc(CNC(=O)c2cccc(Nc3ncc(-c4ccccc4C(F)(F)F)cn3)c2)o1] | ¹H NMR (400 MHz, DMSO-d₆) δ 9.99 (s, 1H), 8.88 (t, J = 5 7 Hz, 1H), 8.48 (s, 2H), 8.25 (s, 1H), 7.97-7.88 (m, 2H), 7.79 (t, J = 7.4 Hz, 1H), 7.67 (t, J = 7.7 Hz, 1H), 7.57-7.45 (m, 2H), 7.39 (t, J = 7.9 Hz, 1H), 6.14 (d, J = 3.0 Hz, 1H), 6.00 (d, J = 2.0 Hz, | 453 | Method A |
| | | 1H), 4.40 (d, J = 5.6 Hz, 2H), 2.24 (s, | | |
| | | 3H). | | |
| 99 | 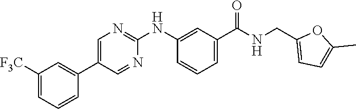 [SMILES: Cc1ccc(CNC(=O)c2cccc(Nc3ncc(-c4cccc(C(F)(F)F)c4)cn3)c2)o1] | ¹H NMR (400 MHz, DMSO-d₆) δ 10.04 (s, 1H), 8.96 (s, 2H), 8.87 (t, J = 5.7 Hz, 1H), 8.28 (dd, J = 1.8. 1.8 Hz, 1H), 8.16-8.01 (m, 2H), 7.94 (dd, J = 1.3, 8.0 Hz, 1H), 7.82-7.66 (m, 2H), 7.47 (d, J = 7.8 Hz, 1H), 7.39 (dd, J = 7.9, 7.9 Hz, 1H), 6.15 (d, | 453 | Method A |
| | | J = 3.0 Hz, 1H), 6.00 (d, J = 2.1 Hz, | | |
| | | 1H), 4.41 (d, J = 5.6 Hz, 2H), 2.24 (s, | | |
| | | 3H). | | |
| 100 | 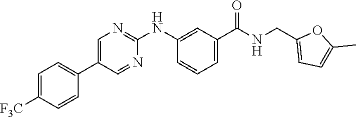 [SMILES: Cc1ccc(CNC(=O)c2cccc(Nc3ncc(-c4ccc(C(F)(F)F)cc4)cn3)c2)o1] | ¹H NMR (400 MHz, DMSO-d₆) δ 10.06 (s, 1H), 8.96 (s, 2H), 8.88 (t, J = 5.7 Hz, 1H), 8.28 (s, 1H), 8.04- 7.90 (m, 3H), 7.8.3 (d, J = 8.4 Hz, 2H), 7.48 (d, J = 7.9 Hz, 1H), 7.40 (dd, J = 7.9, 7.9 Hz, 1H), 6.15 (d, J = 3.0 Hz, 1H), 6.00 (d, J = 2.0 Hz, 1H), 4.41 | 453 | Method A |
| | | (d, J = 5.6 Hz, 2H), 2.24 (s, 3H). | | |
| 101 | 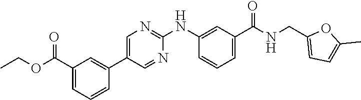 [SMILES: CCOC(=O)c1cccc(-c2cnc(Nc3cccc(C(=O)NCc4ccc(C)o4)c3)nc2)c1] | ¹H NMR (400 MHz, DMSO-d₆) δ 10.01 (s, 1H), 8.91 (s, 2H), 8.89- 8.85 (m, 1H), 8.27 (dd, J = 2.0. 2.0 Hz, 1H), 8.24 (s, 1H), 8.02 (d, J = 8.1 Hz, 1H), 7.99-7.89 (m, 2H), 7.65 (dd, J = 7.8, 7.8 Hz, 1H), 7.47 (d, J = 7.8 Hz, 1H), 7.39 (dd, J = 7.9, 7.9 | 457 | Method A |
| | | Hz, 1H), 6.15 (d, J = 3.0 Hz, 1H), 6.00 | | |
| | | (d, J = 2.0 Hz, 1H), 4.45-4.31 (m, | | |
| | | 4H), 2.24 (s, 3H), 1.36 (t, J = 7.1 Hz, | | |
| | | 3H). | | |
| 102 | 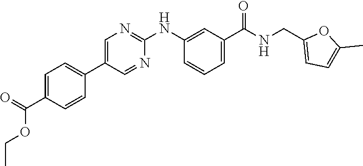 [SMILES: CCOC(=O)c1ccc(-c2cnc(Nc3cccc(C(=O)NCc4ccc(C)o4)c3)nc2)cc1] | ¹H NMR (400 MHz, DMSO-d₆) δ 10.06 (s, 1H), 8.96 (s, 2H), 8.88 (t, J = 5.7 Hz, 1H), 8.28 (t, J = 1.9 Hz, 1H), 8.06-8.03 (m, 2H), 7.95-7.90 (m, 3H), 7.47 (d, J = 7.8 Hz, 1H), 7.39 (t, J = 7.8 Hz, 1H), 6.15 (d, J = 5 .0 Hz, 1H), 6.00 (dd, J = 1.0, 3.0 Hz, 1H), 4.42-4.31 (m, 4H), 2.24 (s, 3H), 1.35 (t, J = 7.1 Hz, 3H). | 457 | Method A |
| 103 | 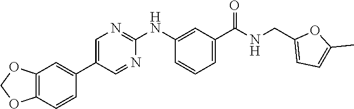 [SMILES: Cc1ccc(CNC(=O)c2cccc(Nc3ncc(-c4ccc5c(c4)OCO5)cn3)c2)o1] | ¹H NMR (400 MHz, DMSO-d₆) δ 9.88 (s, 1H), 8.92-8.77 (m, 3H), 8.25 (dd, J = 2.1, 2.1 Hz, 1H), 7.95- 7.91 (m, 1H), 7.44 (d, J = 7.8 Hz, 1H), 7.40-7.33 (m, 2H), 7.20 (dd, J = 1.8, 8.1 Hz, 1H), 7.03 (d, J = 8.0 Hz, 1H), 6.14 (d, J = 3.0 Hz, 1H), 6.07 (s, 2H), | 429 | Method A |
| | | 6.00 (d, J = 2.0 Hz, 1H), 4.40 (d, | | |
| | | J = 5.6 Hz, 2H), 2.24 (s, 3H). | | |
| 104 | 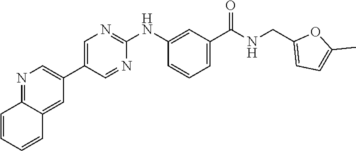 [SMILES: Cc1ccc(CNC(=O)c2cccc(Nc3ncc(-c4cnc5ccccc5c4)cn3)c2)o1] | ¹H NMR (400 MHz, DMSO-d₆) δ 10.07 (s, 1H), 9.32 (d, J = 2.4 Hz, 1H), 9.10 (s, 2H), 8.88 (t, J = 5.7 Hz, 1H), 8.74 (d, J = 2.3 Hz, 1H), 8.30 (s, 1H), 8.04 (dd, J = 7.6, 15.1 Hz, 2H), 7.96 (dd, J = 1.3, 7.9 Hz, 1H), 7.81-7.76 (m, 1H), 7.67 (t, J = 8.1 Hz, 1H), 7.50- 7.46 (m, 1H), 7.41 (t, J = 7.8 Hz, 1H), 6.15 (d, J = 3.0 Hz, 1H), 6.01 (d, | 436 | Method A |
| | | J = 2.0 Hz, 1H), 4.41 (d, J = 5.6 Hz, | | |
| | | 2H), 2.25 (s, 3H). | | |
| 105 | 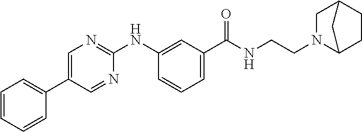 [SMILES: O=C(NCCN1CC2CCC1C2)c1cccc(Nc2ncc(-c3ccccc3)cn2)c1] | ¹H NMR (400 MHz, CDCl₃) δ 8.70 (s, 2H), 8.17 (s, 1H), 7.90 (dd, J = 1.2, 8.1 Hz, 1H), 7.57-7.39 (m, 9H), 3.67-3.56 (m, 3H), 3.06-2.90 (m, 3H), 2.67-2.63 (m, 1H), 2.54 (s, 1H), 1.93-1.80 (m, 2H), 1.72-1.59 (m, 2H), 1.50-1.42 (m, 2H). | 414 | Method B |
| 106 | 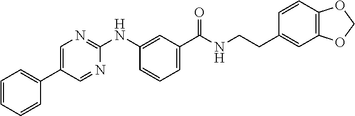 [SMILES: O=C(NCCc1ccc2c(c1)OCO2)c1cccc(Nc2ncc(-c3ccccc3)cn2)c1] | ¹H NMR (400 MHz, DMSO-d₆) δ 9.93 (s, 1H), 8.87 (s, 2H), 8.46 (t, J = 5.6 Hz, 1H), 8.24 (s, 1H), 7.94- 7.90 (m, 1H), 7.73 (d, J = 7.3 Hz, 2H), 7.49 (dd, J = 7.6, 7.6 Hz, 2H), 7.43- 7.32 (m, 3H), 6.87-6.81 (m, 2H), 6.71 (dd, J = 1.6, 7.9 Hz, 1H), 5.97 (s, | 439 | Method B |
| | | 2H), 3.48-3.42 (m, 2H), 2.77 (t, | | |
| | | J = 7.3 Hz, 2H). | | |
| 107 | 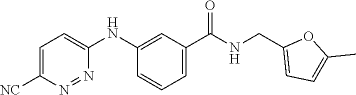 [SMILES: Cc1ccc(CNC(=O)c2cccc(Nc3ccc(C#N)nn3)c2)o1] | ¹H NMR (400 MHz, DMSO-d₆) δ 10.57 (s, 1H), 8.96-8.88 (m, 3H), 8.18 (dd, J = 1.9, 1.9 Hz, 1H), 7.85 (dd, J = 1.7, 7.8 Hz, 1H), 7.57 (d, J = 7.9 Hz, 1H), 7.43 (dd, J = 7.9, 7.9 Hz, 1H), 6.14 (d, J = 3.0 Hz, 1H), 6.00 | 334 | Method A |
| | | (d, J = 2.0 Hz, 1H), 4.40 (d, J = 5.5 Hz, | | |
| | | 2H), 2.24 (s, 3H). | | |
| 108 | 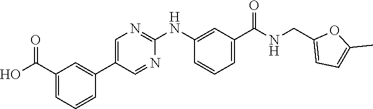 [SMILES: Cc1ccc(CNC(=O)c2cccc(Nc3ncc(-c4cccc(C(=O)O)c4)cn3)c2)o1] | ¹H NMR (400 MHz, DMSO-d₆) δ 13.13 (s, 1H), 9.99 (s, 1H), 8.97- 8.83 (m, 3H), 8.26 (dd, J = 1.8, 1.8 Hz, 1H), 8.22 (s, 1H), 8.06-7.89 (m, 3H), 7.62 (dd, J = 7.7, 7.7 Hz, 1H), 7.46 (d, J = 7.9 Hz, 1H), 7.39 (dd, J = 7.9, 7.9 Hz, 1H), 6.15 (d, J = 3.0 | 429 | Method A |
| | | Hz, 1H), 6.01-6.00 (m, 1H), 4.41 (d, | | |
| | | J = 5.6 Hz, 2H), 2.24 (s, 3H). | | |
| 109 | 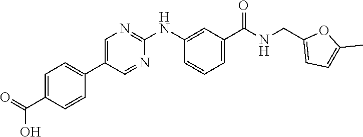 [SMILES: Cc1ccc(CNC(=O)c2cccc(Nc3ncc(-c4ccc(C(=O)O)cc4)cn3)c2)o1] | ¹H NMR (400 MHz, DMSO-d₆) δ 13.02 (s, 1H), 10.04 (s, 1H), 8.95 (s, 2H), 8.87 (t, J = 5.6 Hz, 1H), 8.27 (dd, J = 1.9, 1.9 Hz, 1H), 8.02 (d, J = 8.4 Hz, 2H), 7.98-7.82 (m, 3H), 7.47 (d, J = 7.8 Hz, 1H), 7.39 (dd, J = 7.8, 7.8 Hz, 1H), 6.15 (d, J = 3.0 Hz, 1H), 6.01-5.99 (m, 1H), 4.41 (d, J = 5.5 Hz, 2H), 2.24 (s, 3H). | 429 | Method A |
| 110 | 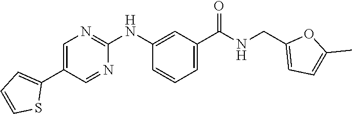 [SMILES: Cc1ccc(CNC(=O)c2cccc(Nc3ncc(-c4cccs4)cn3)c2)o1] | ¹H NMR (400 MHz, DMSO-d₆) δ 10.00 (s, 1H), 8.90-8.85 (m, 1H), 8.82 (s, 2H), 8.26 (dd, J = 1.9, 1.9 Hz, 1H), 7.91 (dd, J = 1.3, 8.0 Hz, 1H), 7.58 (dd, J = 1.0, 5.0 Hz, 1H), 7.54 (dd, J = 1.1, 3.6 Hz, 1H), 7.46 (d, J = 7.8 Hz, 1H), 7.38 (dd, J = 7.9, 7.9 | 391 | Method A |
| | | Hz, 1H), 7.18 (dd, J = 3.6, 5.0 Hz, | | |
| | | 1H), 6.14 (d, J = 3.0 Hz, 1H), 6.00 (d, | | |
| | | J = 2.0 Hz, 1H), 4.40 (d, J = 5.6 Hz, | | |
| | | 2H), 2.24 (s, 3H). | | |
| 111 | 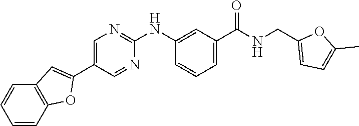 [SMILES: Cc1ccc(CNC(=O)c2cccc(Nc3ncc(-c4cc5ccccc5o4)cn3)c2)o1] | ¹H NMR (400 MHz, DMSO-d₆) δ 10.17 (s, 1H), 9.04 (s, 2H), 8.89 (t, J = 5.7 Hz, 1H), 8.26 (s, 1H), 7.94 (dd, J = 1.4. 8.0 Hz, 1H), 7.72-7.60 (m, 2H), 7.49 (d, J = 7.8 Hz, 1H), 7.46-7.37 (m, 2H), 7.37-7.23 (m, 2H), 6.15 (d, J = 3.0 Hz, 1H), 6.00 (d, J = 2.0 Hz, 1H), 4.41 (d, J = | 425 | Method A |
| | | 5.6 Hz, 2H), 2.25 (s, 3H). | | |
| 112 | 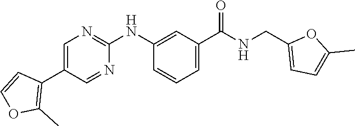 [SMILES: Cc1ccc(CNC(=O)c2cccc(Nc3ncc(-c4ccoc4C)cn3)c2)o1] | ¹H NMR (400 MHz, DMSO-d₆) δ 9.86 (s, 1H), 8.86 (t, J = 5.6 Hz, 1H), 8.64 (s, 2H), 8.26 (dd, J = 18, 1.8 Hz, 1H), 7.92 (dd, J = 1.3, 8.0 Hz, 1H), 7.62 (d, J = 1.9 Hz, 1H), 7.43 (d, J = 7.8 Hz, 1H), 7.37 (dd, J = 7.9, 7.9 Hz, 1H), 6.80 (d, J = 1.9 Hz, 1H), 6.14 (d, J = 2.9 Hz, 1H), 6.00 (dd, J = 1.0, | 389 | Method A |
| | | 3.0 Hz, 1H), 4.40 (d, J = 5.6 Hz, 2H), | | |
| | | 2.45 (s, 3H), 2.24 (s, 3H). | | |
| 113 | 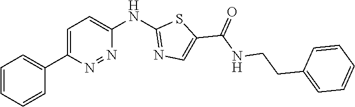 [SMILES: O=C(NCCc1ccccc1)c1cnc(Nc2ccc(-c3ccccc3)nn2)s1] | ¹H NMR (400 MHz, DMSO-d₆) δ 8.89-8.84 (m, 1H), 8.25-8.23 (m, 1H), 8.02-7.94 (m, 2H), 7.81 (t, J = 8.7 Hz, 1H), 7.58 (m, 3H), 7.35- 7.21 (m, 7H), 3.51-3.45 (m, 2H), 2.89-2.83 (m, 2H). | 402 | Method A |
| 114 | 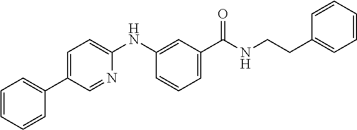 [SMILES: O=C(NCCc1ccccc1)c1cccc(Nc2ccc(-c3ccccc3)cn2)c1] | ¹H NMR (400 MHz, DMSO-d₆) δ 9.35 (s, 1H), 8.56-8.46 (m, 2H), 8.09 (d, J = 1.9 Hz, 1H), 7.97-7.91 (m, 2H), 7.72-7.61 (m, 2H), 7.51- 7.42 (m, 2H), 7.40-7.16 (m, 8H), 6.95 (d, J = 8.6 Hz, 1H), 3.53-3.45 (m, 2H), 2.86 (t, J = 7.4 Hz, 2H). | 394 | Method A |
| 115 | 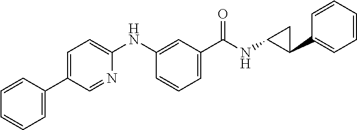 [SMILES: O=C(N[C@@H]1C[C@H]1c1ccccc1)c1cccc(Nc2ccc(-c3ccccc3)cn2)c1] | ¹H NMR (400 MHz, DMSO-d₆) δ 9.37 (s, 1H), 8.65 (d, J = 4.5 Hz, 1H), 8.53 (d, J = 2.5 Hz, 1H), 8.07 (d, J = 1.1 Hz, 1H), 8.01-7.97 (m, 1H), 7.93 (dd, J = 2.6, 8.7 Hz, 1H), 7.71- 7.55 (m, 2H), 7.46 (dd, J = 7.8, 7.8 Hz, 2H), 7.42-7.24 (m, 5H), 7.23- 7.13 (m, 3H), 6.95 (d, J = 8.8 Hz, | 406 | Method A |
| | | 1H), 3.09-3.03 (m, 1H), 2.12-2.06 | | |
| | | (m, 1H), 1.41-1.34 (m, 1H), | | |
| | | 1.27-1.20 (m, 1H). | | |
| 115-1 | 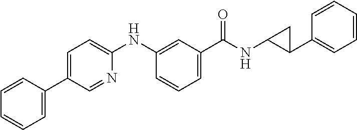 [SMILES: O=C(NC1CC1c1ccccc1)c1cccc(Nc2ccc(-c3ccccc3)cn2)c1] | — | — | — |
| 116 | 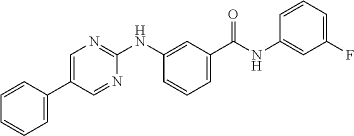 [SMILES: O=C(Nc1cccc(F)c1)c1cccc(Nc2ncc(-c3ccccc3)cn2)c1] | ¹H NMR (400 MHz, DMSO-d₆) δ 10.46 (s, 1H), 10.04 (s, 1H), 8.89 (s, 2H), 8.35 (dd, J = 2.0, 2.0 Hz, 1H), 8.03 (d, J = 8.0 Hz, 1H), 7.83-7.71 (m, 3H), 7.64-7.28 (m, 7H), 6.98- 6.91 (m, 1H). | 385 | Method B |
| 117 | 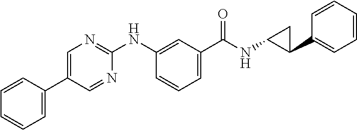 [SMILES: O=C(N[C@@H]1C[C@H]1c1ccccc1)c1cccc(Nc2ncc(-c3ccccc3)cn2)c1] | ¹H NMR (400 MHz, DMSO-d₆) δ 9.95 (s, 1H), 8.87 (s, 2H), 8.66 (d, J = 4.5 Hz, 1H), 8.25 (s, 1H), 7.97- 7.92 (m, 1H), 7.76-7.72 (m, 2H), 7.49 (t, J = 7.6 Hz, 2H), 7.41-7.36 (m, 3H), 7.29 (t, J = 7.6 Hz, 2H), 7.21-7.16 (m, 3H), 3.08-3.01 (m, 1H), 2.12-2.05 (m, 1H), 1.41-1.34 (m, | 407 | Method B |
| | | 1H), 1.27-1.20 (m, 1H). | | |
| 117-1 | 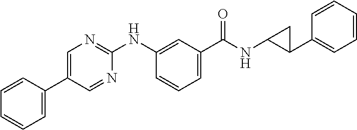 [SMILES: O=C(NC1CC1c1ccccc1)c1cccc(Nc2ncc(-c3ccccc3)cn2)c1] | — | — | — |
| 118 | 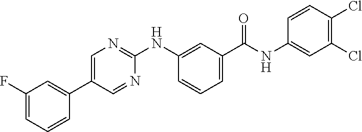 [SMILES: O=C(Nc1ccc(Cl)c(Cl)c1)c1cccc(Nc2ncc(-c3cccc(F)c3)cn2)c1] | ¹H NMR (400 MHz, DMSO-d₆) δ 10.53 (s, 1H), 10.11 (s, 1H), 8.94 (s, 2H), 8.37 (dd, J = 2.0, 2.0 Hz, 1H), 8.18 (d, J = 2.5 Hz, 1H), 8.02 (d, J = 8.1 Hz, 1H), 7.78 (dd, J = 2.4, 8.9 Hz, 1H), 7.73-7.58 (m, 3H), 7.58-7.32 (m, 3H), 7.24-7.18 (m, 1H). | 453 | Method B |
| 119 | 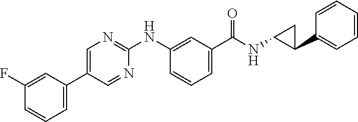 [SMILES: O=C(N[C@@H]1C[C@H]1c1ccccc1)c1cccc(Nc2ncc(-c3cccc(F)c3)cn2)c1] | ¹H NMR (400 MHz, DMSO-d₆) δ 10.00 (s, 1H), 8.92 (s, 2H), 8.66 (d, J = 4.5 Hz, 1H), 8.24 (dd, J = 1.9, 1.9 Hz, 1H), 7.95-7.92 (m, 1H), 7.71- 7.57 (m, 2H), 7.55-7.49 (m, 1H), 7.47-7.35 (m, 2H), 7.32-7.27 (m, 2H), 7.24-7.16 (m, 4H), 3.08-3.01 (m, 1H), 2.12-2.05 (m, 1H), 1.40- | 425 | Method B |
| | | 1.34 (m, 1H), 1.27-1.20 (m, 1H). | | |
| 119-1 | 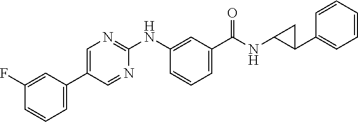 [SMILES: O=C(NC1CC1c1ccccc1)c1cccc(Nc2ncc(-c3cccc(F)c3)cn2)c1] | — | — | — |
| 120 | 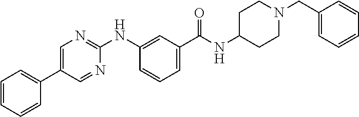 [SMILES: O=C(NC1CCN(Cc2ccccc2)CC1)c1cccc(Nc2ncc(-c3ccccc3)cn2)c1] | ¹H NMR (400 MHz, DMSO-d₆) δ 9.91 (s, 1H), 8.86 (s, 2H), 8.30-8.12 (m, 2H), 7.98-7.94 (m, 1H), 7.77- 7.69 (m, 2H), 7.49 (dd, J = 7.6, 7.6 Hz, 2H), 7.45-7.19 (m, 8H), 3.82- 3.73 (m, 1H), 3.48 (s, 2H), 2.84 (d, J = 10.8 Hz, 2H), 2.07-1.99 (m, 2H), | 464 | Method B |
| | | 1.80 (d, J = 10.4 Hz, 2H), 1.59 (ddd, | | |
| | | J = 3.3, 11.9, 23.5 Hz, 2H). | | |
| 121 | 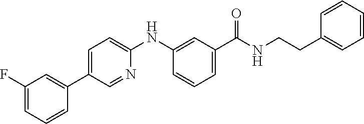 [SMILES: O=C(NCCc1ccccc1)c1cccc(Nc2ccc(-c3cccc(F)c3)cn2)c1] | ¹H NMR (400 MHz, DMSO-d₆) δ 9.41 (s, 1H), 8.57 (d, J = 2.5 Hz, 1H), 8.51 (dd, J = 5.6, 5.6 Hz, 1H), 8.09 (s, 1H), 8.01-7.86 (m, 2H), 7.65-7.43 (m, 3H), 7.40-7.10 (m, 8H), 6.94 (d, J = 8.8 Hz, 1H), 3.52-3.45 (m, 2H), 2.86 (t, J = 7.4 Hz, 2H). | 412 | Method B |
| 122 | 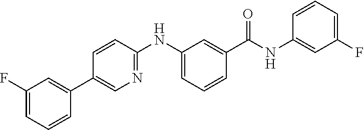 [SMILES: O=C(Nc1cccc(F)c1)c1cccc(Nc2ccc(-c3cccc(F)c3)cn2)c1] | ¹H NMR (400 MHz, DMSO-d₆) δ 10.45 (s, 1H), 9.51 (s, 1H), 8.60 (d, J = 2.5 Hz, 1H), 8.20 (d, J= 1.6 Hz, 1H), 8.08-7.94 (m, 2H), 7.80-7.70 (m, 1H), 7.62-7.35 (m, 7H), 7.19- 7.13 (m. 1H), 7.04-6.89 (m, 2H). | 402 | Method B |
| 123 | 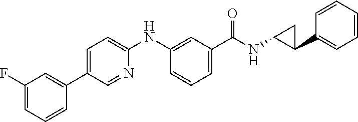 [SMILES: O=C(N[C@@H]1C[C@H]1c1ccccc1)c1cccc(Nc2ccc(-c3cccc(F)c3)cn2)c1] | ¹H NMR (400 MHz, DMSO-d₆) δ 9.42 (s, 1H), 8.65 (d, J = 4.5 Hz, 1H), 8.58 (d, J = 2.5 Hz, 1H), 8.08 (s, 1H), 8.01-7.96 (m, 2H), 7.61-7.43 (m, 3H), 7.42-7.33 (m, 2H), 7.33-7.24 (m, 2H), 7.23-7.03 (m, 4H), 6.94 (d, J = 8.6 Hz, 1H), 3.09-3.02 (m, 1H), 2.11-2.05 (m, 1H), 1.41-1.34 (m, | 424 | Method B |
| | | 1H), 1.27-1.16 (m, 1H). | | |
| 123-1 | 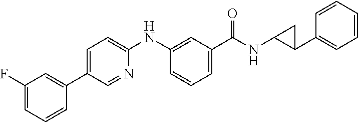 [SMILES: O=C(NC1CC1c1ccccc1)c1cccc(Nc2ccc(-c3cccc(F)c3)cn2)c1] | — | — | — |
| 124 | 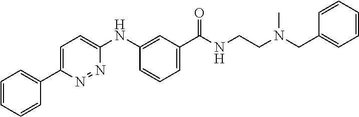 [SMILES: CN(CCNC(=O)c1cccc(Nc2ccc(-c3ccccc3)nn2)c1)Cc1ccccc1] | ¹H NMR (400 MHz, DMSO-d₆) δ 9.57 (s, 1H), 8.38 (t, J = 5.6 Hz, 1H), 8.22-8.19 (m, 1H), 8.09-7.99 (m, 4H), 7.55-7.50 (m, 2H), 7.49-7.41 (m, 3H), 7.33-7.22 (m, 6H), 3.54- 3.52 (m, 2H), 3.46-3.41 (m, 2H), 2.56-2.53 (m, 2H), 2.21-2.20 (m, | 438 | Method B |
| | | 3H). | | |
| 125 | 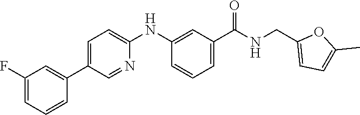 [SMILES: Cc1ccc(CNC(=O)c2cccc(Nc3ccc(-c4cccc(F)c4)cn3)c2)o1] | ¹H NMR (400 MHz, DMSO-d₆) δ 9.42 (s, 1H), 8.87 (t, J = 5.7 Hz, 1H), 8.58 (d, J = 2.3 Hz, 1H), 8.10 (d, J = 1.8 Hz, 1H), 8.03-7.91 (m, 2H), 7.61-7.44 (m, 3H), 7.44-7.33 (m, 2H), 7.19-7.12 (m, 1H), 6.94 (d, J = 8.8 Hz, 1H), 6.14 (d, J = 3.0 Hz, | 402 | Method B |
| | | 1H), 6.00 (dd, J = 1.0, 3.0 Hz, 1H), | | |
| | | 4.41 (d, J = 5.6 Hz, 2H), 2.24 (s, 3H). | | |
| 126 | 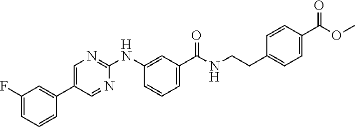 [SMILES: COC(=O)c1ccc(CCNC(=O)c2cccc(Nc3ncc(-c4cccc(F)c4)cn3)c2)cc1] | ¹H NMR (400 MHz, DMSO-d₆) δ 10.01 (s, 1H), 8.91 (s, 2H), 8.53 (dd, J = 5.6, 5.6 Hz, 1H), 8.23 (s, 1H), 8.03-7.87 (m, 3H), 7.69-7.58 (m, 2H), 7.56-7.49 (m, 1H), 7.46-7.30 (m, 4H), 7.24-7.18 (m, 1H), 3.83 (s, 3H), 3.53 (q, J = 6.6 Hz, 2H), 2.94 (t, J = 7.3 Hz, 2H). | 471 | Method B |
| 127 | 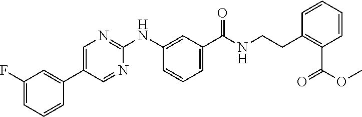 [SMILES: COC(=O)c1ccccc1CCNC(=O)c1cccc(Nc2ncc(-c3cccc(F)c3)cn2)c1] | ¹H NMR (400 MHz, DMSO-d₆) δ 10.00 (s, 1H), 8.91 (s, 2H), 8.50 (dd, J = 5.5, 5.5 Hz, 1H), 8.23 (s, 1H), 7.94-7.89 (m, 1H), 7.82-7.80 (m, 1H), 7.72-7.57 (m, 2H), 7.57-7.46 (m, 2H), 7.43-7.30 (m, 4H), 7.24-7.18 (m, 1H), 3.84 (s, 3H), 3.55-3.48 (m, | 471 | Method B |
| | | 2H), 3.17 (t, J = 7.1 Hz, 2H). | | |
| 128 | 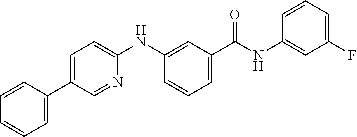 [SMILES: O=C(Nc1cccc(F)c1)c1cccc(Nc2ccc(-c3ccccc3)cn2)c1] | ¹H NMR (400 MHz, DMSO-d₆) δ 10.45 (s, 1H), 9.46 (s, 1H), 8.55 (d, J = 2.5 Hz, 1H), 8.20 (s, 1H), 8.07- 8.03 (m, 1H), 7.95 (dd, J = 2.6, 8.7 Hz, 1H), 7.80-7.75 (m, 1H), 7.67 (d, J = 7.3 Hz, 2H), 7.59 (dd, J = 1.5, 7.8 Hz, 1H), 7.51-7.29 (m, 6H), 7.02-6.85 (m, 2H). | 384 | Method B |
| 129 | 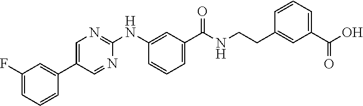 [SMILES: O=C(O)c1cccc(CCNC(=O)c2cccc(Nc3ncc(-c4cccc(F)c4)cn3)c2)c1] | ¹H NMR (400 MHz, DMSO-d₆) δ 12.92 (s, 1H), 10.00 (s, 1H), 8.91 (s, 2H), 8.53 (t, J = 5.6 Hz, 1H), 8.24 (d, J = 1.1 Hz, 1H), 7.94-7.90 (m, 1H), 7.85 (s, 1H), 7.80 (d, J = 7.6 Hz, 1H), 7.68-7.59 (m, 2H), 7.56-7.49 (m, 2H), 7.46-7.36 (m, 3H), 7.23-7.18 | 457 | Method B |
| | | (m, 1H), 3.50 (q, J = 6.7 Hz, 2H), | | |
| | | 2.92 (t, J = 7.3 Hz, 2H). | | |
| 130 | 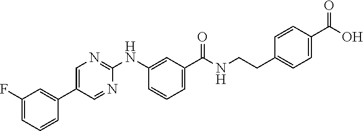 [SMILES: O=C(O)c1ccc(CCNC(=O)c2cccc(Nc3ncc(-c4cccc(F)c4)cn3)c2)cc1] | ¹H NMR (400 MHz, DMSO-d₆) δ 12.85 (s, 1H), 10.01 (s, 1H), 8.91 (s, 2H), 8.53 (dd, J = 5.6, 5.6 Hz, 1H), 8.23 (s, 1H), 7.96-7.85 (m, 3H), 7.72-7.57 (m, 2H), 7.56-7.49 (m, 1H), 7.43-7.34 (m, 4H), 7.23-7.18 (m, 1H), 3.56-3.48 (m, 2H), 2.94 (t, J = 7.2 Hz, 2H). | 457 | Method B |
| 131 | 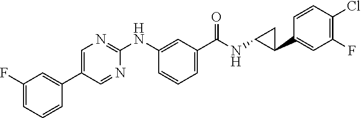 [SMILES: O=C(N[C@@H]1C[C@H]1c1ccc(Cl)c(F)c1)c1cccc(Nc2ncc(-c3cccc(F)c3)cn2)c1] | ¹H NMR (400 MHz, DMSO-d₆) δ 10.02 (s, 1H), 8.92 (s, 2H), 8.70 (d, J = 4.4 Hz, 1H), 8.25 (s, 1H), 7.95- 7.91 (m, 1H), 7.68-7.59 (m, 2H), 7.55-7.37 (m, 4H), 7.27-7.18 (m, 2H), 7.09 (dd, J = 1.9, 8.3 Hz, 1H), 3.10-3.03 (m, 1H), 2.14-2.08 (m, 1H), 1.47-1.40 (m, 1H), 1.34-1.28 | 477 | Method B |
| | | (m, 1H). | | |
| 131-1 | 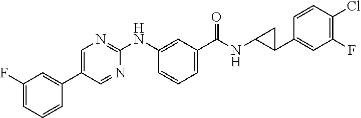 [SMILES: O=C(NC1CC1c1ccc(Cl)c(F)c1)c1cccc(Nc2ncc(-c3cccc(F)c3)cn2)c1] | — | — | — |
| 132 | 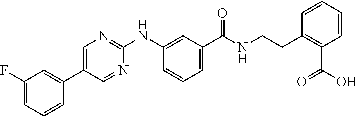 [SMILES: O=C(NCCc1ccccc1C(=O)O)c1cccc(Nc2ncc(-c3cccc(F)c3)cn2)c1] | ¹H NMR (400 MHz, DMSO-d₆) δ 12.97 (s, 1H), 9.99 (s, 1H), 8.91 (s, 2H), 8.54 (s, 1H), 8.22 (s, 1H), 7.93- 7.88 (m, 1H), 7.83-7.81 (m, 1H), 7.68-7.59 (m, 2H), 7.56-7.46 (m, 2H), 7.39-7.31 (m, 4H), 7.23-7.18 (m, 1H), 3.53 (q, J = 6.5 Hz, 2H), 3.20 (t, J = 7.0 Hz, 2H). | 457 | Method B |
| 133 | 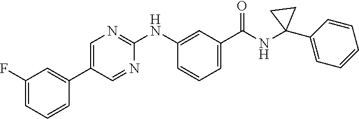 [SMILES: O=C(NC1(c2ccccc2)CC1)c1cccc(Nc2ncc(-c3cccc(F)c3)cn2)c1] | ¹H NMR (400 MHz, DMSO-d₆) δ 10.01 (s, 1H), 9.15 (s, 1H), 8.91 (s, 2H), 8.26 (dd, J = 1.8, 1.8 Hz, 1H), 7.97 (dd, J = 1.4, 8.1Hz, 1H), 7.67- 7.59 (m, 2H), 7.55-7.49 (m, 2H), 7.40 (t, J = 7.9 Hz, 1H), 7.29 (dd, J = 7.6,7.6 Hz, 2H), 7.23-7.15 (m, 4H), 1.29-1.26 (m, 4H). | 425 | Method B |
| 134 | 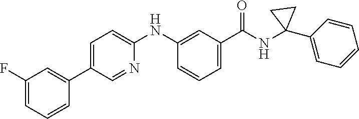 [SMILES: O=C(NC1(c2ccccc2)CC1)c1cccc(Nc2ccc(-c3cccc(F)c3)cn2)c1] | ¹H NMR (400 MHz, DMSO-d₆) δ 9.43 (s, 1H), 9.14 (s, 1H), 8.57 (d, J = 2.3 Hz, 1H), 8.09-7.95 (m, 3H), 7.57-7.44 (m, 4H), 7.38 (dd, J = 7.8, 7.8 Hz, 1H), 7.32-7.26 (m, 2H), 7.22-7.12 (m, 4H), 6.94 (d, J = 8.4 Hz, 1H), 1.29-1.26 (m, 4H). | 424 | Method B |
| 135 | 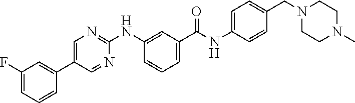 [SMILES: CN1CCN(Cc2ccc(NC(=O)c3cccc(Nc4ncc(-c5cccc(F)c5)cn4)c3)cc2)CC1] | ¹H NMR (400 MHz, DMSO-d₆) δ 10.25 (s, 1H), 10.08 (s, 1H), 8.94 (s, 2H), 8.34 (dd, J = 1.9, 1.9 Hz, 1H), 7.99 (dd, J = 1.2, 8.1 Hz, 1H), 7.73 (d, J = 8.7 Hz, 2H), 7.69-7.60 (m, 2H), 7.56-7.44 (m, 3H), 7.27 (d, J = 8.5 Hz, 2H), | 497 | Method B |
| | | 7.24-7.18 (m, 1H), 3.42 (s, | | |
| | | 2H), 2.35 (s, 8H), 2.17 (s, 3H). | | |
| 136 | 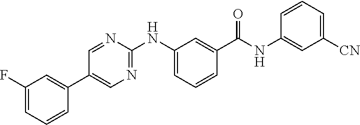 [SMILES: N#Cc1cccc(NC(=O)c2cccc(Nc3ncc(-c4cccc(F)c4)cn3)c2)c1] | ¹H NMR (400 MHz, DMSO-d₆) δ 10.61 (s, 1H), 10.12 (s, 1H), 8.94 (s, 2H), 8.38 (dd, J = 1.8, 1.8 Hz, 1H), 8.29-8.27 (m, 1H), 8.09-8.05 (m, 1H), 8.04-8.01 (m, 1H), 7.69-7.47 (m, 7H), 7.24-7.19 (m, 1H). | 410 | Method B |
| 137 | 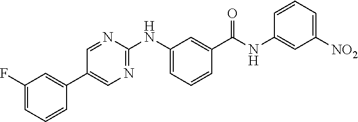 [SMILES: O=C(Nc1cccc([N+](=O)[O-])c1)c1cccc(Nc2ncc(-c3cccc(F)c3)cn2)c1] | ¹H NMR (400 MHz, DMSO-d₆) δ 10.74 (s, 1H), 10.13 (s, 1H), 8.95 (s, 2H), 8.84 (dd, J = 2.1, 2.1 Hz, 1H), 8.42 (s, 1H), 8.21 (dd, J = 1.3. 8.3 Hz, 1H), 8.01 (ddd, J = 3.1, 10.4, 14.7 Hz, 2H), 7.70-7.59 (m, 4H), 7.56-7.48 (m, 2H), 7.24-7.18 (m, 1H). | 430 | Method B |
| 138 | 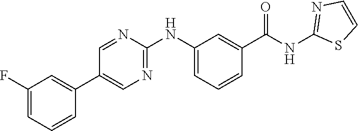 [SMILES: O=C(Nc1nccs1)c1cccc(Nc2ncc(-c3cccc(F)c3)cn2)c1] | ¹H NMR (400 MHz, DMSO-d₆) δ 12.62 (s, 1H), 10.12 (s, 1H), 8.96 (s, 2H), 8.52 (dd, J = 1.8. 1.8 Hz, 1H), 8.00 (dd, J = 1.9, 8.0 Hz, 1H), 7.74 (d, J = 8.3 Hz, 1H), 7.69-7.61 (m, 2H), 7.58 (d, J = 3.3 Hz, 1H), 7.56-7.45 (m, 2H), 7.30 (d, J = 3.5 Hz, 1H), 7.24-7.18 (m, 1H). | 392 | Method B |
| 139 | 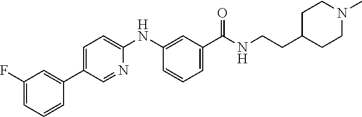 [SMILES: CN1CCC(CCNC(=O)c2cccc(Nc3ccc(-c4cccc(F)c4)cn3)c2)CC1] | ¹H NMR (400 MHz, DMSO-d₆) δ 9.42 (s, 1H), 9.17 (s, 1H), 8.57 (d, J = 2.4 Hz, 1H), 8.41 (t, J = 5.6 Hz, 1H), 8.11 (s, 1H), 8.00-7.92 (m, 2H), 7.57-7.45 (m, 3H), 7.38-7.35 (m, 2H), 7.19-7.12 (m, 1H), 6.94 (d, J = 8.6 Hz, 1H), 2.91-2.84 (m, 2H), | 433 | Method B |
| | | 2.73 (s, 3H), 1.93 (d, J = 17.5 Hz, | | |
| | | 2H), 1.50 (s, 3H), 1.32 (s, 2H). | | |
| 140 | 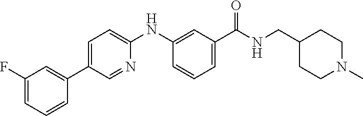 [SMILES: CN1CCC(CNC(=O)c2cccc(Nc3ccc(-c4cccc(F)c4)cn3)c2)CC1] | ¹H NMR (400 MHz, DMSO-d₆) δ 9.45-9.42 (m, 2H), 8.57 (d, J = 2.4 Hz, 1H), 8.52 (t, J = 5.6 Hz, 1H), 8.10 (s, 1H), 8.00-7.95 (m, 2H), 7.57- 7.46 (m, 3H), 7.37 (d, J = 5.0 Hz, 2H), 7.19-7.13 (m, 1H), 6.95 (d, J = 8.6 Hz, 1H), 3.19 (s, 2H), 2.92-2.89 (m, | 419 | Method B |
| | | 2H), 2.73 (s, 3H), 1.92-1.80 (m, | | |
| | | 3H), 1.41-1.39 (m, 2H). | | |
| 141 | 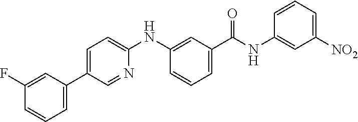 [SMILES: O=C(Nc1cccc([N+](=O)[O-])c1)c1cccc(Nc2ccc(-c3cccc(F)c3)cn2)c1] | ¹H NMR (400 MHz, DMSO-d₆) δ 10.73 (s, 1H), 9.53 (s, 1H), 8.84 (dd, J = 2.1, 2.1 Hz, 1H), 8.61 (d, J = 2.4 Hz, 1H), 8.27 (dd, J = 1.8, 1.8 Hz, 1H), 8.21 (dd, J = 1.2, 8.2 Hz, 1H), 8.07-8.03 (m, 1H), 8.02-7.96 (m, 2H), 7.68 (dd, J = 8.2, 8.2 Hz, 1H), 7.57-7.45 (m, 5H), 7.19-7.13 (m, | 429 | Method B |
| | | 1H), 6.98 (d, J = 8.8 Hz, 1H). | | |
| 142 | 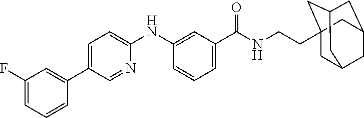 [SMILES: O=C(NCCC12CC3CC(CC(C3)C1)C2)c1cccc(Nc2ccc(-c3cccc(F)c3)cn2)c1] | ¹H NMR (400 MHz, DMSO-d₆) δ 9.40 (s, 1H), 8.57 (d, J = 2.5 Hz, 1H), 8.30 (t, J = 5.5 Hz, 1H), 8.07 (s, 1H), 7.99-7.94 (m, 2H), 7.56-7.46 (m, 3H), 7.38-7.32 (m, 2H), 7.18-7.12 (m, 1H), 6.94 (d, J = 8.8 Hz, 1H), 3.31-3.24 (m, 2H), 1.95 (s, 3H), 1.70 (d, J = 12.4 Hz, 6H), 1.54 (d, J = 2.3 | 470 | Method B |
| | | Hz, 6H), 1.34 (td, J = 4.1, 8.1 Hz, 2H). | | |
| 143 | 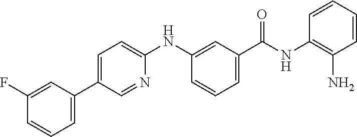 [SMILES: Nc1ccccc1NC(=O)c1cccc(Nc2ccc(-c3cccc(F)c3)cn2)c1] | ¹H NMR (400 MHz, DMSO-d₆) δ 9.65 (s, 1H), 9.47 (s, 1H), 8.59 (d, J = 2.5 Hz, 1H), 8.19 (dd, J = 2.0, 2.0 Hz, 1H), 8.04 (dd, J = 1.8, 8.1 Hz, 1H), 7.98 (dd, J = 2.7, 8.8 Hz, 1H), 7.57-7.41 (m, 5H), 7.21 (d, J = 8.6 Hz, 1H), 7.18-7.12 (m, 1H), 7.01- 6.96 (m, 2H), 6.80 (dd, J = 1.4. 8.0 Hz, 1H), 6.65-6.59 (m, 1H), 4.91 (s, | 399 | Method B |
| | | 2H). | | |
| 144 | 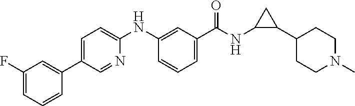 [SMILES: CN1CCC(C2CC2NC(=O)c2cccc(Nc3ccc(-c4cccc(F)c4)cn3)c2)CC1] | ¹H NMR (400 MHz, DMSO-d₆) δ 9.39 (s, 1H), 8.58 (d, J = 2.5 Hz, 1H), 8.36 (d, J = 4.3 Hz, 1H), 8.04 (s, 1H), 7.99-7.93 (m, 2H), 7.57-7.45 (m, 3H), 7.37-7.31 (m, 2H), 7.18-7.12 (m, 1H), 6.95-6.92 (m, 1H), 2.74 (t, J = 9.3 Hz, 2H), 2.64-2.59 (m, 1H), | 445 | Method B |
| | | 2.13 (s, 3H), 1.88-1.75 (m, 3H), | | |
| | | 1.60 (d, J =12.8 Hz, 1H), 1.42-1.22 | | |
| | | (m, 2H), 0.80-0.64 (m, 3H), 0.59- | | |
| | | 0.53 (m, 1H). | | |
| 145 | 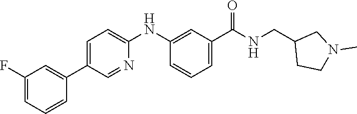 [SMILES: CN1CCC(CNC(=O)c2cccc(Nc3ccc(-c4cccc(F)c4)cn3)c2)C1] | ¹H NMR (400 MHz, DMSO-d₆) δ 9.41 (s, 1H), 8.57 (d, J = 2.5 Hz, 1H), 8.50 (dd, J = 5.8, 5.8 Hz, 1H), 8.07 (s, 1H), 8.00-7.96 (m, 2H), 7.56-7.46 (m, 3H), 7.40-7.34 (m, 2H), 7.19- 7.12 (m, 1H), 6.94 (d, J = 8.6 Hz, 1H), 3.25 (t, J = 6.2 Hz, 2H), 2.72- | 405 | Method B |
| | | 2.65 (m, 2H), 2.59-2.55 (m, 1H), | | |
| | | 2.47-2.44 (m, 1H), 2.39-2.33 (m, | | |
| | | 3H), 1.98-1.88 (m, 2H), 1.59-1.48 | | |
| | | (m, 1H). | | |
| 146 | 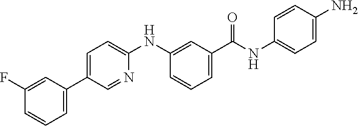 [SMILES: Nc1ccc(NC(=O)c2cccc(Nc3ccc(-c4cccc(F)c4)cn3)c2)cc1] | ¹H NMR (400 MHz, DMSO-d₆) δ 9.86 (s, 1H), 9.46 (s, 1H), 8.59 (d, J = 2.4 Hz, 1H), 8.13 (s, 1H), 8.03- 7.96 (m, 2H), 7.57-7.37 (m. 7H), 7.19-7.12 (m, 1H), 6.96 (d, J = 8.8 Hz, 1H), 6.55 (d, J = 8.6 Hz, 2H), 4.94 (s, 2H). | 399 | Method B |
| 147 | 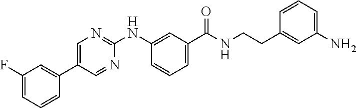 [SMILES: Nc1cccc(CCNC(=O)c2cccc(Nc3ncc(-c4cccc(F)c4)cn3)c2)c1] | ¹H NMR (400 MHz, DMSO-d₆) δ 10.00 (s, 1H), 8.92-8.91 (m, 2H), 8.49 (dd, J = 5.6, 5.6 Hz, 1H), 8.25 (dd, J = 1.8, 1.8 Hz, 1H), 7.94-7.90 (m, 1H), 7.68-7.63 (m, 1H), 7.61 (d, J = 7.6 Hz, 1H), 7.56-7.49 (m, 1H), 7.43-7.36 (m, 2H), 7.23-7.18 (m, | 428 | Method B |
| | | 1H), 6.94 (dd, J = 7.6, 7.6 Hz, 1H), | | |
| | | 6.46 (dd, J = 2.0, 2.0 Hz, 1H), 6.43- | | |
| | | 6.39 (m, 2H), 4.99 (s, 2H), 3.46- | | |
| | | 3.39 (m, 2H), 2.71-2.65 (m, 2H). | | |
| 148 | 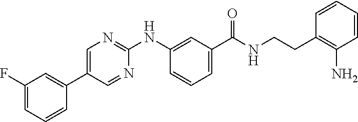 [SMILES: Nc1ccccc1CCNC(=O)c1cccc(Nc2ncc(-c3cccc(F)c3)cn2)c1] | ¹H NMR (400 MHz, DMSO-d₆) δ 10.01 (s, 1H), 8.93 (s, 2H), 8.61 (dd, J = 5.6, 5.6 Hz, 1H), 8.30 (s, 1H), 7.94-7.90 (m, 1H), 7.68-7.63 (m, 1H), 7.61 (d, J = 6.3 Hz, 1H), 7.53 (dt, J = 6.7, 8.2 Hz, 1H), 7.45-7.37 (m, 2H), 7.23-7.18 (m, 1H). 6.97-6.90 (m, 2H), 6.64 (d, J = 7.0 Hz, 1H), | 428 | Method B |
| | | 6.52-6.47 (m, 1H), 5.12 (s, 2H), | | |
| | | 3.42-3.36 (m, 2H), 2.70 (dd, J = | | |
| | | 7.7, 7.7 Hz, 2H). | | |
| 149 | 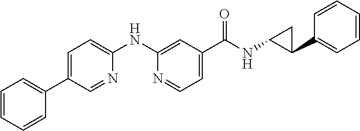 [SMILES: O=C(N[C@@H]1C[C@H]1c1ccccc1)c1ccnc(Nc2ccc(-c3ccccc3)cn2)c1] | ¹H NMR (400 MHz, DMSO-d₆) δ 10.07 (s, 1H), 8.90 (d, J = 4.4 Hz, 1H), 8.61 (d, J = 2.3 Hz, 1H), 8.36 (d, J = 5.3 Hz, 1H), 8.10 (s, 1H), 8.04 (dd, J = 2.3, 8.8 Hz, 1H), 7.85-7.81 (m, 1H), 7.72-7.69 (m, 2H), 7.48 (t, J = 7.7 Hz, 2H), 7.36 (t, J = 7.4 Hz, 1H), 7.30 (t, J = 7.6 Hz, 2H), 7.24- | 407 | Method B |
| | | 7.17 (m, 4H), 3.09-3.02 (m, 1H), | | |
| | | 2.15-2.08 (m, 1H), 1.41-1.35 (m, | | |
| | | 1H), 1.29- 1.23 (m, 1H). | | |
| 149-1 | 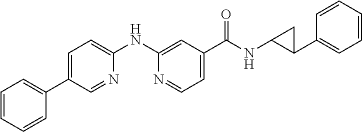 [SMILES: O=C(NC1CC1c1ccccc1)c1ccnc(Nc2ccc(-c3ccccc3)cn2)c1] | — | — | — |
| 150 | 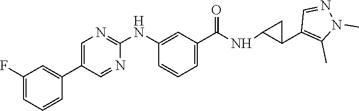 [SMILES: Cc1c(C2CC2NC(=O)c2cccc(Nc3ncc(-c4cccc(F)c4)cn3)c2)cnn1C] | ¹H NMR (400 MHz, DMSO-d₆) δ 9.99 (s, 1H), 8.92 (s, 2H), 8.57 (d, J = 4.0 Hz, 1H), 8.24 (dd, J = 1.9, 1.9 Hz, 1H), 7.94 (dd, J = 2.0, 7.8 Hz, 1H), 7.66 (td, J = 2.0, 10.5 Hz, 1H), 7.61 (d, J = 8.4 Hz, 1H), 7.56-7.49 (m, 1H), 7.44-7.36 (m, 2H), 7.23- | 443 | Method B |
| | | 7.18 (m, 1H), 7.08 (s, 1H), 3.69 (s, | | |
| | | 3H), 2.80-2.74 (m, 1H), 2.32-2.31 | | |
| | | (m, 3H), 1.77-1.71 (m, 1H), 1.20- | | |
| | | 1.13 (m, 1H), 1.03-0.98 (m, 1H). | | |
| 151 | 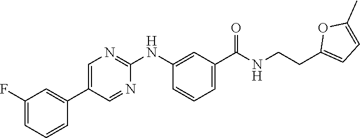 [SMILES: Cc1ccc(CCNC(=O)c2cccc(Nc3ncc(-c4cccc(F)c4)cn3)c2)o1] | ¹H NMR (400 MHz, DMSO-d₆) δ 10.00 (s, 1H), 8.91 (s, 2H), 8.53 (t, J = 5.6 Hz, 1H), 8.24 (s, 1H). 7.95- 7.91 (m, 1H), 7.68-7.63 (m, 1H), 7.61 (d, J = 8.2 Hz, 1H), 7.56-7.49 (m, 1H), 7.41-7.36 (m, 2H), 7.24- 7.18 (m, 1H), 6.05 (d, J = 3.0 Hz, 1H), 5.95 (d, J = 2.0 Hz, 1H), 3.52-3.45 (m, 2H), 2.82 (t, J = | 417 | Method B |
| | | 7.3 Hz, 2H), 2.22 (s, 3H). | | |
| 152 | | ¹H NMR (400 MHz, DMSO-d₆) δ 9.93 (s, 1H), 8.87 (s, 2H), 8.57 (d, J = 4.0 Hz. 1H), 8.24 (s, 1H), 7.97- 7.93 (m, 1H), 7.74 (d, J = 7.3 Hz, 2H), 7.49 (t, J = 7.6 Hz, 2H), 7.44- 7.36 (m, 3H), 7.08 (s, 1H), 3.69 (s, 3H), 2.80-2.74 (m, 1H), 2.32-2.31 (m ,3H), 1.78-1.71 (m, 1H), 1.20- 1.14 (m, 1H), 1.04-0.97 (m, 1H). | 425 | Method B |
| 153 | | ¹H NMR (400 MHz, DMSO-d₆) δ 9.94 (s, 1H), 8.87-8.86 (m, 2H), 8.53 (t, J = 5.6 Hz, 1H), 8.24 (s, 1H), 7.96-7.92 (m, 1H), 7.74 (d, J = 7.4 Hz, 2H), 7.49 (t, J = 7.7 Hz, 2H), 7.41-7.35 (m, 3H), 6.05 (d, J = 3.0 Hz, 1H), 5.95 (d, J = 2.1 Hz, 1H), 3.52-3.45 (m, 2H), 2.82 (t, J = 7.3 Hz, 2H), 2.22 (s. 3H). | 399 | Method B |
| 154 | 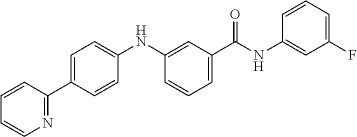 [SMILES: O=C(Nc1cccc(F)c1)c1cccc(Nc2ccc(-c3ccccn3)cc2)c1] | ¹H NMR (400 MHz, DMSO-d₆) δ 10.43 (s, 1H), 8.71 (s, 1H), 8.61- 8.60 (m, 1H), 8.05-8.01 (m, 2H), 7.93-7.72 (m, 3H), 7.68 (s, 1H), 7.60-7.54 (m, 1H), 7.50-7.30 (m, 4H), 7.28-7.20 (m, 3H), 6.97-6.90 (m, 1H). | 384 | Method B |
| 155 | 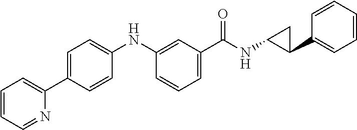 [SMILES: O=C(N[C@@H]1C[C@H]1c1ccccc1)c1cccc(Nc2ccc(-c3ccccn3)cc2)c1] | ¹H NMR (400 MHz, DMSO-d₆) δ 8.67 (d, J = 4.4 Hz, 1H), 8.64-8.59 (m, 2H), 8.03-7.99 (m, 2H), 7.89- 7.80 (m, 2H), 7.64-7.61 (m, 1H), 7.38-7.25 (m, 6H), 7.19-7.14 (m, 5H), 3.07-3.01 (m, 1H), 2.11-2.05 (m, 1H), 1.39-1.33 (m, 1H), 1.25- 1.16 (m, 1H). | 406 | Method B |
| 155-1 | 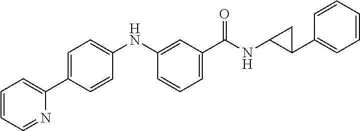 [SMILES: O=C(NC1CC1c1ccccc1)c1cccc(Nc2ccc(-c3ccccn3)cc2)c1] | — | — | — |
| 156 | | ¹H NMR (400 MHz, DMSO-d₆) δ 10.16 (s, 1H), 9.04 (d, J = 2.5 Hz, 1H), 8.92 (s, 2H), 8.88 (d, J = 4.4 Hz, 1H), 8.66 (dd, J = 2.2, 2.2 Hz, 1H), 8.60 (d, J = 1.8 Hz, 1H), 7.75 (d, J = 7.3 Hz, 2H), 7.50 (dd, J = 7.7, 7.7 Hz, 2H), 7.40 (t, J = 7.4 Hz, 1H), 7.33-7.27 (m, 2H), 7.22-7.17 (m, 3H), 3.09- 3.03 (m, 1H), 2.15-2.09 (m, 1H), 1.41-1.35 (m, 1H), 1.29-1.23 (m, 1H). | 408 | Method B |
| 156-1 | 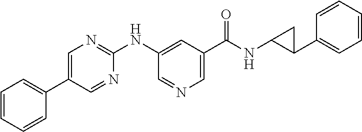 [SMILES: O=C(NC1CC1c1ccccc1)c1cncc(Nc2ncc(-c3ccccc3)cn2)c1] | — | — | — |
| 157 | 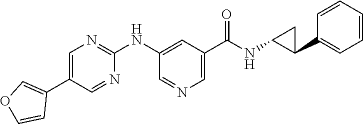 [SMILES: O=C(N[C@@H]1C[C@H]1c1ccccc1)c1cncc(Nc2ncc(-c3ccoc3)cn2)c1] | ¹H NMR (400 MHz, DMSO-d₆) δ 9.87 (s, 1H), 8.81 (s, 2H), 8.63 (d, J = 4.5 Hz, 1H), 8.24 (d, J = 4.3 Hz, 2H), 7.89 (d, J = 7.6 Hz, 1H), 7.79 (s, 1H), 7.42-7.36 (m, 2H), 7.32-7.27 (m, 2H), 7.21-7.17 (m, 3H), 7.04 (d, J = 1.0 Hz, 1H), 3.07-3.01 (m, 1H), 2.12-2.05 (m, 1H), 1.40-1.34 (m, | 397 | Method B |
| | | 1H), 1.27-1.20 (m, 1H). | | |
| 157-1 | 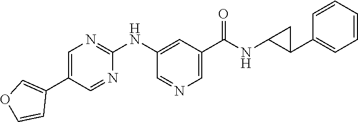 [SMILES: O=C(NC1CC1c1ccccc1)c1cncc(Nc2ncc(-c3ccoc3)cn2)c1] | — | — | — |
| 158 | 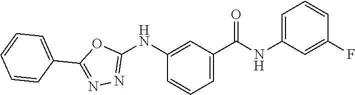 [SMILES: O=C(Nc1cccc(F)c1)c1cccc(Nc2nnc(-c3ccccc3)o2)c1] | ¹H NMR (400 MHz, DMSO-d₆) δ 10.96 (s, 1H), 10.51 (s, 1H), 8.16- 8.13 (m, 1H), 8.06-7.83 (m, 3H), 7.78 (d, J = 11.9 Hz, 1H), 7.70-7.49 (m, 6H), 7.41 (q, J = 7.7 Hz, 1H), 6.98-6.92 (m, 1H). | 375 | Method B |
| 159 | 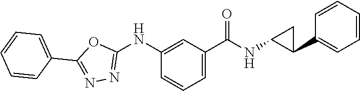 [SMILES: O=C(N[C@@H]1C[C@H]1c1ccccc1)c1cccc(Nc2nnc(-c3ccccc3)o2)c1] | ¹H NMR (400 MHz, DMSO-d₆) δ 10.87 (s, 1H), 8.73 (d, J = 4.4 Hz, 1H), 8.05 (d, J = 1.0 Hz, 1H), 7.94-7.90 (m, 2H), 7.82-7.78 (m, 1H), 7.61- 7.57 (m, 3H), 7.52-7.41 (m, 2H), 7.32-7.27 (m, 2H), 7.24-7.13 (m, | 397 | Method B |
| | | 3H), 3.08-3.02 (m, 1H), 2.13-2.07 | | |
| | | (m, 1H), 1.41-1.34 (m, 1H), 1.28- | | |
| | | 1.21 (m, 1H). | | |
| 159-1 | 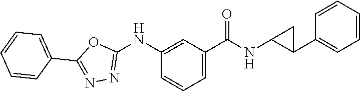 [SMILES: O=C(NC1CC1c1ccccc1)c1cccc(Nc2nnc(-c3ccccc3)o2)c1] | — | — | — |
| 160 | 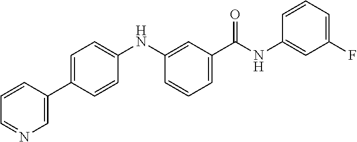 [SMILES: O=C(Nc1cccc(F)c1)c1cccc(Nc2ccc(-c3cccnc3)cc2)c1] | ¹H NMR (400 MHz, DMSO-d₆) δ 10.42 (s, 1H), 8.87 (s, 1H), 8.67 (s, 1H), 8.50 (d, J = 4.6 Hz, 1H), 8.06- 8.02 (m, 1H), 7.79-7.74 (m, 1H), 7.70-7.65 (m, 3H), 7.59-7.55 (m, 1H), 7.48-7.33 (m, 5H), 7.27-7.23 (m, 2H), 6.97-6.91 (m, 1H). | 384 | Method B |
| 161 | 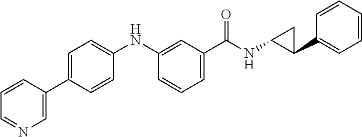 [SMILES: O=C(N[C@@H]1C[C@H]1c1ccccc1)c1cccc(Nc2ccc(-c3cccnc3)cc2)c1] | ¹H NMR (400 MHz, DMSO-d₆) δ 8.87 (d, J = 2.3 Hz, 1H), 8.66 (d, J = 4.3 Hz, 1H), 8.57 (s, 1H), 8.50 (dd, J = 1.2, 4.6 Hz, 1H), 8.03 (td, J = 1.9, 8.1 Hz, 1H), 7.68-7.60 (m, 3H), 7.45 (dd, J = 4.8, 7.9 Hz, 1H), 7.37- 7.25 (m, 5H), 7.18 (dd, J = 8.4, 15.8 Hz, 5H), 3.07-3.01 (m, 1H), 2.11- | 406 | Method B |
| | | 2.05 (m, 1H), 1.39-1.33 (m, 1H), | | |
| | | 1.25-1.19 (m, 1H). | | |
| 161-1 | 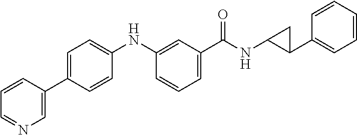 [SMILES: O=C(NC1CC1c1ccccc1)c1cccc(Nc2ccc(-c3cccnc3)cc2)c1] | — | — | — |
| 162 | 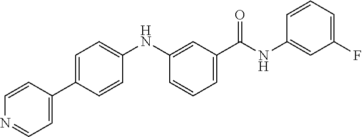 [SMILES: O=C(Nc1cccc(F)c1)c1cccc(Nc2ccc(-c3ccncc3)cc2)c1] | ¹H NMR (400 MHz, DMSO-d₆) δ 10.42 (s, 1H), 8.76 (s, 1H), 8.57 (d, J = 5.3 Hz, 2H), 7.79-7.73 (m, 3H), 7.71-7.65 (m, 3H), 7.59-7.55 (m, 1H), 7.48-7.35 (m, 4H), 7.26-7.23 (m, 2H), 6.97-6.91 (m, 1H). | 384 | Method B |
| 163 | 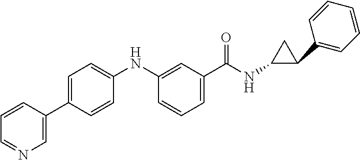 [SMILES: O=C(N[C@@H]1C[C@H]1c1ccccc1)c1cccc(Nc2ccc(-c3cccnc3)cc2)c1] | ¹H NMR (400 MHz, DMSO-d₆) δ 8.68-8.64 (m, 2H), 8.55 (s, 2H), 7.78-7.73 (m, 2H), 7.70-7.62 (m, 3H), 7.39-7.35 (m, 2H), 7.32-7.26 (m, 3H), 7.22-7.15 (m, 5H), 3.07- 3.01 (m, 1H), 2.11-2.05 (m, 1H), 1.40-1.33 (m, 1H), 1.26-1.19 (m, 1H). | 406 | Method B |
| 163-1 | 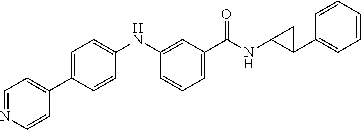 [SMILES: O=C(NC1CC1c1ccccc1)c1cccc(Nc2ccc(-c3ccncc3)cc2)c1] | — | — | — |
| 164 | 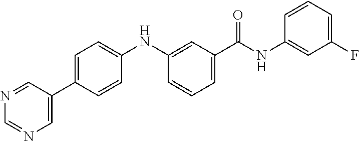 [SMILES: O=C(Nc1cccc(F)c1)c1cccc(Nc2ccc(-c3cncnc3)cc2)c1] | ¹H NMR (400 MHz, DMSO-d₆) δ 10.43 (s, 1H), 9.12-9.11 (m, 3H), 8.74 (s, 1H), 7.78-7.73 (m, 3H), 7.70-7.68 (m, 1H), 7.59-7.55 (m, 1H), 7.47-7.33 (m, 4H), 7.29-7.24 (m, 2H), 6.97-6.91 (m, 1H). | 385 | Method B |
| 165 | 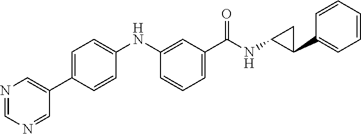 [SMILES: O=C(N[C@@H]1C[C@H]1c1ccccc1)c1cccc(Nc2ccc(-c3cncnc3)cc2)c1] | ¹H NMR (400 MHz, DMSO-d₆) δ 9.11 (d, J = 1.4 Hz, 3H), 8.69-8.64 (m, 2H), 7.76-7.71 (m, 2H), 7.63 (s, 1H), 7.38-7.36 (m, 2H), 7.32-7.25 (m, 3H), 7.23-7.14 (m, 5H), 3.05- 3.02 (m. 1H), 2.10-2.04 (m, 1H), 1.39-1.34 (m, 1H), 1.25-1.16 (m, 1H). | 407 | Method B |
| 165-1 | 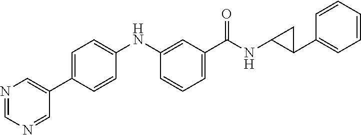 [SMILES: O=C(NC1CC1c1ccccc1)c1cccc(Nc2ccc(-c3cncnc3)cc2)c1] | — | — | — |
| 166 | 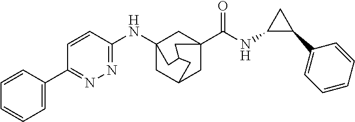 [SMILES: O=C(N[C@@H]1C[C@H]1c1ccccc1)C12CC3CC(CC(Nc4ccc(-c5ccccc5)nn4)(C3)C1)C2] | ¹H NMR (400 MHz, DMSO-d₆) δ 7.97 (d, J = 7.4 Hz, 2H), 7.78-7.75 (m, 1H), 7.68 (d, J = 4.3 Hz, 1H), 7.47 (t, J = 7.5 Hz, 2H), 7.39 (t, J = 7.3 Hz, 1H), 7.25 (t, J = 7.5 Hz, 2H), 7.17- 7.08 (m, 3H), 6.92-6.89 (m, 1H), 6.63 (s, 1H), 2.85-2.79 (m, 1H), 2.19 (s, 4H), 2.16-2.07 (m, 4H), | 465 | Method B |
| | | 1.96-1.89 (m, 1H), 1.83-1.71 (m, | | |
| | | 4H), 1.69-1.58 (m, 2H), 1.27-1.19 | | |
| | | (m, 1H), 1.13-1.07 (m, 1H). | | |
| 166-1 | 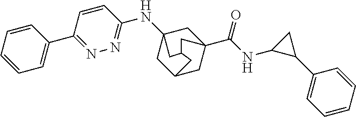 [SMILES: O=C(NC1CC1c1ccccc1)C12CC3CC(CC(Nc4ccc(-c5ccccc5)nn4)(C3)C1)C2] | — | — | — |
| 167 | 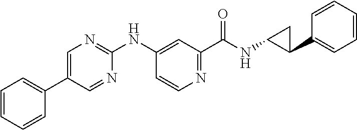 [SMILES: O=C(N[C@@H]1C[C@H]1c1ccccc1)c1cc(Nc2ncc(-c3ccccc3)cn2)ccn1] | ¹H NMR (400 MHz, DMSO-d₆) δ 10.52 (s, 1H), 8.99 (s, 2H), 8.97 (d, J = 5.8 Hz, 1H), 8.48 (d, J = 2.2 Hz, 1H), 8.45 (d, J = 5.6 Hz, 1H), 8.05 (dd, J = 2.3, 5.6 Hz, 1H), 7.80-7.76 (m, 2H), 7.51 (dd, J = 7.6, 7.6 Hz, 2H), 7.42 (t, J = 7.4 Hz, 1H), 7.29 (dd, J = 7.6, 7.6 Hz, 2H), 7.20-7.16 | 408 | Method B |
| | | (m, 3H), 3.11-3.04 (m, 1H), 2.21- | | |
| | | 2.15 (m. 1H), 1.53-1.47 (m 1H), | | |
| | | 1.28-1.22 (m, 1H). | | |
| 167-1 | 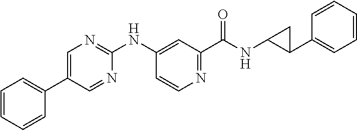 [SMILES: O=C(NC1CC1c1ccccc1)c1cc(Nc2ncc(-c3ccccc3)cn2)ccn1] | — | — | — |
| 168 | 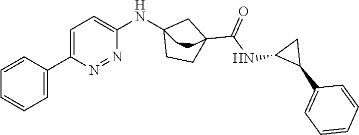 | ¹H NMR (400 MHz, DMSO-d₆) δ 8.00-7.96 (m, 2H), 7.81 (dd, J = 2.9, 6.9 Hz, 2H), 7.48 (dd, J = 7.5, 7.5 Hz, 2H), 7.42-7.38 (m, 1H), 7.26 (dd, J = 7.5, 7.5 Hz, 2H), 7.19-7.10 (m, 4H), 6.92 (d, J = 9.4 Hz, 1H), 2.90- 2.84 (m, 2H), 2.17-2.06 (m, 4H), 1.98-1.94 (m, 2H), 1.85-1.73 (m, 2H), 1.73-1.64 (m, 2H), 1.25 (ddd, | 425 | Method B |
| | | J = 6.1, 6.1, 6.1 Hz, 1H), 1.14-1.08 | | |
| | | (m, 1H). | | |
| 168-1 | 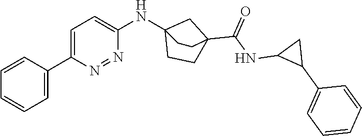 [SMILES: O=C(NC1CC1c1ccccc1)C12CCC(Nc3ccc(-c4ccccc4)nn3)(CC1)C2] | — | — | — |
| 170 | 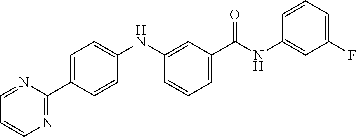 [SMILES: O=C(Nc1cccc(F)c1)c1cccc(Nc2ccc(-c3ncccn3)cc2)c1] | ¹H NMR (400 MHz, DMSO-d₆) δ 10.44 (s, 1H), 8.85 (s, 1H), 8.82 (d, J = 4.8 Hz, 2H), 8.33-8.29 (m, 2H), 7.76 (td, J = 2.2, 11.8 Hz, 1H), 7.72- 7.70 (m, 1H), 7.59-7.55 (m, 1H), 7.51-7.45 (m, 2H), 7.42-7.36 (m, 2H), 7.32 (t, J = 4.8 Hz, 1H), 7.24- 7.20 (m, 2H), 6.94 (ddd, J = 8.4, 8.4, 2.4 Hz, 1H). | 385 | Method B |
| 171 | 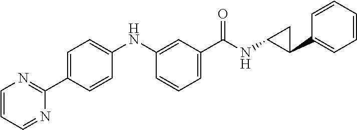 [SMILES: O=C(N[C@@H]1C[C@H]1c1ccccc1)c1cccc(Nc2ccc(-c3ncccn3)cc2)c1] | ¹H NMR (400 MHz, DMSO-d₆) δ 8.82 (d, J = 4.8 Hz, 2H), 8.77 (s, 1H), 8.68 (d, J = 4.5 Hz, 1H), 8.31-8.28 (m, 2H), 7.65-7.64 (m, 1H), 7.43- 7.23 (m, 6H), 7.20-7.15 (m, 5H), 3.08-3.01 (m, 1H), 2.11-2.05 (m, 1H), 1.40-1.33 (m, 1H), 1.26-1.19 (m, 1H). | 407 | Method B |
| 171-1 | 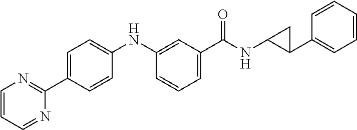 [SMILES: O=C(NC1CC1c1ccccc1)c1cccc(Nc2ccc(-c3ncccn3)cc2)c1] | — | — | — |
| 172 | 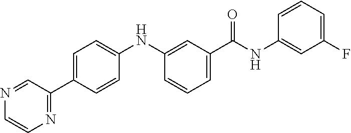 [SMILES: O=C(Nc1cccc(F)c1)c1cccc(Nc2ccc(-c3cnccn3)cc2)c1] | ¹H NMR (400 MHz, DMSO-d₆) δ 10.44 (s, 1H), 9.18 (d, J = 1.4Hz, 1H), 8.82 (s, 1H), 8.65-8.63 (m, 1H), 8.50 (d, J = 2.5 Hz, 1H), 8.11-8.07 (m, 2H), 7.76 (td, J = 2.1, 11.6 Hz, 1H), 7.72-7.70 (m, 1H), 7.59-7.55 (m, 1H), 7.53-7.33 (m, 4H), 7.27- 7.23 (m, 2H), 6.97-6.91 (m, 1H). | 385 | Method B |
| 173 | 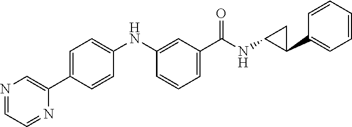 [SMILES: O=C(N[C@@H]1C[C@H]1c1ccccc1)c1cccc(Nc2ccc(-c3cnccn3)cc2)c1] | ¹H NMR (400 MHz, DMSO-d₆) δ 9.17 (d, J = 1.5 Hz, 1H), 8.73 (s, 1H), 8.68 (d, J = 4.3 Hz, 1H), 8.65-8.62 (m, 1H), 8.49 (d, J = 2.5 Hz, 1H), 8.10-8.04 (m, 2H), 7.66-7.64 (m. 1H), 7.40-7.37 (m, 2H), 7.33-7.26 (m, 3H), 7.22-7.15 (m, 5H), 3.07-3.01 (m. 1H), 2.11-2.05 (m, 1H), 1.39- | 407 | Method B |
| | | 1.33 (m, 1H), 1.26-1.19 (m, 1H). | | |
| 173-1 | 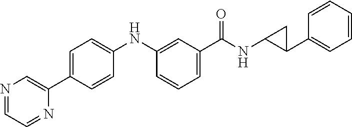 [SMILES: O=C(NC1CC1c1ccccc1)c1cccc(Nc2ccc(-c3cnccn3)cc2)c1] | — | — | — |
| 174 | 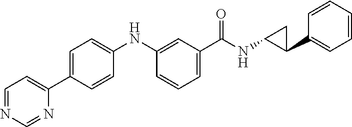 [SMILES: O=C(N[C@@H]1C[C@H]1c1ccccc1)c1cccc(Nc2ccc(-c3ccncn3)cc2)c1] | ¹H NMR (400 MHz, DMSO-d₆) δ 9.13 (d, J = 1.3 Hz, 1H), 8.85 (s, 1H), 8.74 (d, J = 5.5 Hz, 1H), 8.69 (d, J = 4.4 Hz, 1H), 8.16-8.13 (m, 2H), 7.96 (dd, J = 1.3, 5.6 Hz, 1H), 7.68- 7.65 (m, 1H), 7.44-7.37 (m, 2H), 7.35-7.26 (m, 3H), 7.21-7.15 (m, 5H), 3.07-3.01 (m, 1H), 2.11-2.05 | 407 | Method B |
| | | (m, 1H), 1.39-1.33 (m, 1H), 1.26- | | |
| | | 1.20 (m, 1H). | | |
| 174-1 | 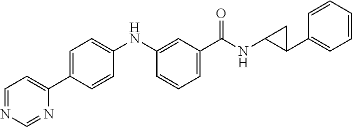 [SMILES: O=C(NC1CC1c1ccccc1)c1cccc(Nc2ccc(-c3ccncn3)cc2)c1] | — | — | — |
| 175 | 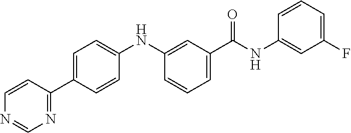 [SMILES: O=C(Nc1cccc(F)c1)c1cccc(Nc2ccc(-c3ccncn3)cc2)c1] | ¹H NMR (400 MHz, DMSO-d₆) δ 10.45 (s, 1H), 9.14 (s, 1H), 8.94 (s, 1H). 8.75 (d, J = 5.4 Hz, 1H), 8.16 (d, J = 8.8 Hz, 2H), 7.99-7.96 (m, 1H), 7.78-7.72 (m, 2H), 7.59-7.45 (m, 3H), 7.43-7.36 (m, 2H), 7.26-7.22 (m, 2H), 6.98-6.91 (m, 1H). | 385 | Method B |
| 176 | 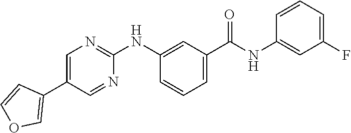 [SMILES: O=C(Nc1cccc(F)c1)c1cccc(Nc2ncc(-c3ccoc3)cn2)c1] | ¹H NMR (400 MHz, DMSO-d₆) δ 10.45 (s, 1H), 9.98 (s, 1H), 8.84 (s, 2H), 8.37 (t, J = 1.9 Hz, 1H), 8.25 (s, 1H), 7.98-7.95 (m, 1H), 7.81-7.74 (m, 2H), 7.60-7.56 (m, 1H), 7.54- 7.36 (m, 3H), 7.06-7.04 (m, 1H), 6.98-6.91 (m, 1H). | 375 | Method B |
| 177 | 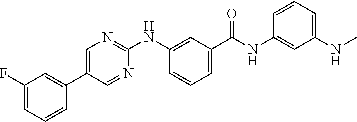 [SMILES: CNc1cccc(NC(=O)c2cccc(Nc3ncc(-c4cccc(F)c4)cn3)c2)c1] | ¹H NMR (400 MHz, DMSO-d₆) δ 10.06 (s, 1H), 9.99 (s, 1H), 8.94 (s, 2H), 8.31 (s, 1H), 8.00-7.96 (m, 1H), 7.68-7.59 (m, 2H), 7.56-7.49 (m, 2H), 7.45 (t, J = 7.8 Hz, 1H), 7.21 (dt = 2.0, 8.5 Hz, 1H), 7.09- 7.01 (m, 2H), 6.96 (d, J = 8.0 Hz, | 414 | Method B |
| | | 1H) ,6.30 (dd, J = 1.1, 7.9 Hz, 1H), | | |
| | | 5.68 (q, J = 4.8 Hz, 1H), 2.68 | | |
| | | (d, J = 5.0 Hz, 3H). | | |
| 178 | 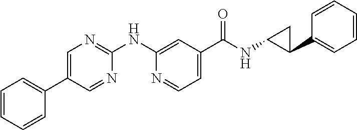 [SMILES: O=C(N[C@@H]1C[C@H]1c1ccccc1)c1ccnc(Nc2ncc(-c3ccccc3)cn2)c1] | ¹H NMR (400 MHz, DMSO-d₆) δ 10.26 (s, 1H), 8.96 (s, 2H), 8.93 (d, J = 4.5 Hz, 1H), 8.64 (s, 1H), 8.42 (d, J = 5.0 Hz, 1H), 7.77 (d, J = 7.4 Hz, 2H), 7.51 (dd, J = 7.6, 7.6 Hz, 2H), 7.41 (t, J = 7.4 Hz, 1H), 7.35 (dd, J = 1.3, 5.1 Hz, 1H), 7.33-7.27 (m, 2H), 7.22-7.18 (m, 3H), 3.09-3.03 | 408 | Method B |
| | | (m, 1H), 2.15-2.09 (m, 1H), 1.42- | | |
| | | 1.35 (m, 1H), 1.30-1.23 (m, 1H). | | |
| 178-1 | 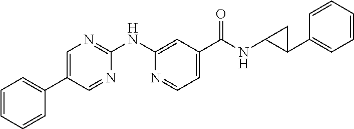 [SMILES: O=C(NC1CC1c1ccccc1)c1ccnc(Nc2ncc(-c3ccccc3)cn2)c1] | — | — | — |
| 179 | 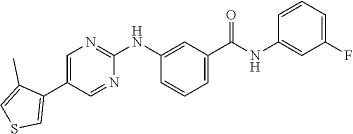 [SMILES: Cc1cscc1-c1cnc(Nc2cccc(C(=O)Nc3cccc(F)c3)c2)nc1] | ¹H NMR (400 MHz, DMSO-d₆) δ 10.45 (s, 1H), 9.99 (s, 1H), 8.64 (s, 2H), 8.33 (s, 1H), 8.04-8.01 (m, 1H), 7.77 (td, J = 3.5, 7.1 Hz, 1H), 7.63 (d, J = 3.3 Hz, 1H), 7.60-7.51 (m, 2H), 7.47 (t, J = 7.9 Hz, 1H), 7.43-7.36 (m, 1H), 7.34-7.33 (m, 1H), 6.98-6.91 (m, 1H), 2.29 (s, 3H). | 405 | Method B |
| 180 | 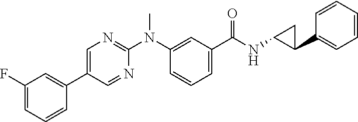 [SMILES: CN(c1cccc(C(=O)N[C@@H]2C[C@H]2c2ccccc2)c1)c1ncc(-c2cccc(F)c2)cn1] | ¹H NMR (400 MHz, DMSO-d₆) δ 8.80 (s, 2H), 8.72 (d, J = 4.3 Hz, 1H), 7.85 (dd, J = 1.8. 1.8 Hz, 1H), 7.75- 7.72 (m, 1H), 7.60-7.47 (m, 5H), 7.29 (dd, J = 7.5, 7.5 Hz, 2H), 7.21- 7.15 (m, 4H), 3.56 (s, 3H), 3.08- 3.01 (m, 1H), 2.12-2.06 (m, 1H), 1.39-1.33 (m, 1H), 1.27-1.21 (m, | 439 | Method E |
| | | 1H). | | |
| 180-1 | 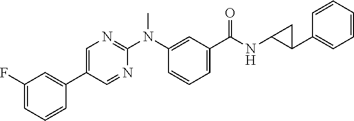 [SMILES: CN(c1cccc(C(=O)NC2CC2c2ccccc2)c1)c1ncc(-c2cccc(F)c2)cn1] | — | — | — |
| 181 | 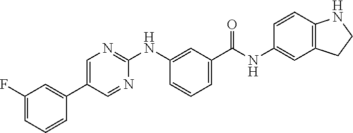 [SMILES: O=C(Nc1ccc2c(c1)CCN2)c1cccc(Nc2ncc(-c3cccc(F)c3)cn2)c1] | 1H NMR (400 MHz, DMSO-d₆) δ 10.04 (s, 1H), 9.89 (s, 1H), 8.93 (s, 2H), 8.29 (dd, J = 2.0, 2.0 Hz, 1H), 7.99-7.95 (m, 1H), 7.66 (td, J = 3.6, 7.2 Hz, 1H), 7.61 (d, J = 7.9 Hz, 1H), 7.56-7.48 (m, 2H), 7.47-7.41 (m, 2H), 7.28-7.17 (m, 2H), 6.48 (d, J = 8.3 Hz, 1H), 5.38 (s, 1H), 3.45- | 426 | Method B |
| | | 3.38 (m, 2H), 2.92 (t, J = 8.4 Hz, 2H). | | |
| 182 | 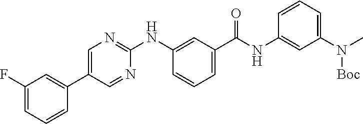 [SMILES: CN(C(=O)OC(C)(C)C)c1cccc(NC(=O)c2cccc(Nc3ncc(-c4cccc(F)c4)cn3)c2)c1] | ¹H NMR (400 MHz, DMSO-d₆) δ 10.29 (s, 1H), 10.08 (s, 1H), 8.94 (s, 2H), 8.35 (t, J = 18 Hz, 1H), 8.00 (dd, J = 3.8, 3.8 Hz, 1H), 7.78 (t, J = 2.1 Hz, 1H), 7.66 (td, J = 3.7, 7.3 Hz, 1H), 7.61 (d, J = 8.1 Hz, 2H), 7.56-7.45 (m, 3H), 7.33 (t, J = 8.4 Hz, 1H), 7.21 (dt, J = 2.0, 8.5 Hz, 1H), 7.02 (dd, | 514 | Method B |
| | | J = 1.4, 8.0 Hz, 1H), 3.20 (s, 3H), | | |
| | | 1.42 (s, 9H). | | |
| 183 | 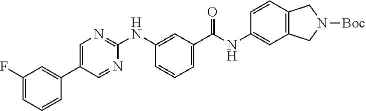 [SMILES: CC(C)(C)OC(=O)N1Cc2ccc(NC(=O)c3cccc(Nc4ncc(-c5cccc(F)c5)cn4)c3)cc2C1] | ¹H NMR (400 MHz, DMSO-d₆) δ 10.31 (d, J = 2.8 Hz, 1H), 10.08 (s, 1H), 8.94 (s, 2H), 8.36-8.32 (m. 1H), 8.00 (d, J = 8.0 Hz, 1H), 7.82 (d, J = 19.8 Hz, 1H), 7.69-7.59 (m, 3H), 7.56-7.44 (m, 3H), 7.31 (dd, J = 5.8, 8.2 Hz. 1H), 7.24- 7.19 (m, 1H), | 526 | Method B |
| | | 4.58 (dd, J = 11.2, 14.7 Hz, 4H), 1.47 | | |
| | | (s, 9H). | | |
| 184 | 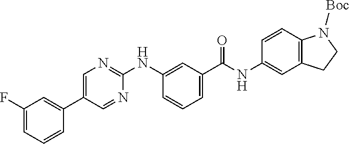 [SMILES: CC(C)(C)OC(=O)N1CCc2cc(NC(=O)c3cccc(Nc4ncc(-c5cccc(F)c5)cn4)c3)ccc21] | ¹H NMR (400 MHz, DMSO-d₆) δ 10.15 (s, 1H), 10.07 (s, 1H), 8.93 (s, 2H), 8.32 (s, 1H), 8.01-7.96 (m, 1H), 7.69-7.44 (m, 8H), 7.21 (dt, J = 2.4, 8.5 Hz, 1H), 3.92 (t, J = 8.6 Hz, 2H), 3.08 (t, J = 8.4 Hz, 2H), 1.52 (s, 9H). | 526 | Method B |
| 185 | 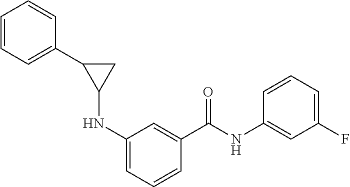 [SMILES: O=C(Nc1cccc(F)c1)c1cccc(NC2CC2c2ccccc2)c1] | ¹H NMR (400 MHz, DMSO-d₆) δ 10.21 (s, 1H), 7.66 (d, J = 11.6 Hz, 1H), 7.52-7.47 (m, 1H), 7.37-7.27 (m, 1H), 7.27-7.03 (m, 7H), 6.90- 6.82 (m, 1H), 6.72 (d, J = 7.0 Hz, 1H), 6.57 (s, 1H), 1.87 (s, 1H), 1.30- 1.23 (m, 1H), 1.22-1.13 (m, 1H). | 347 | Method B |
| 186 | 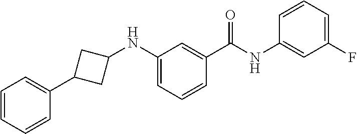 [SMILES: O=C(Nc1cccc(F)c1)c1cccc(NC2CC(c3ccccc3)C2)c1] | ¹H NMR (400 MHz, DMSO-d₆) δ 10.30 (s, 1H), 7.77 (d, J = 11.7 Hz, 1H), 7.58 (d, J = 7.3 Hz, 1H), 7.46- 7.17 (m, 7H), 7.17-7.01 (m, 2H), 6.93 (t, J = 7.7 Hz, 1H), 6.76 (dd, J = 7.7, 20.7 Hz, 1H), 6.44 -6.24 (m, 1H), 4.01-3.88 (m, 1H), 3.30-3.18 (m, 1H), 2.91-2.78 (m, 2H), 2.03- 1.88 (m, 2H). | 361 | Method E |
| 187 | 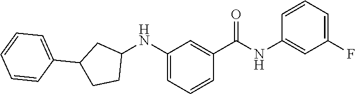 [SMILES: O=C(Nc1cccc(F)c1)c1cccc(NC2CCC(c3ccccc3)C2)c1] | ¹H NMR (400 MHz, DMSO-d₆) δ 10.30 (s, 1H), 7.77 (d, J = 11.6 Hz, 1H), 7.58 (d, J = 8.1 Hz, 1H), 7.40 (q, J = 7.6 Hz, 1H), 7.35-7.28 (m, 4H), 7.27-7.15 (m, 2H), 7.13-7.06 (m, 2H), 6.93 (t, J = 7.9 Hz, 1H), 6.85- | 375 | Method E |
| | | 6.77 (m, 1H), 6.13-6.05 (m, 1H), | | |
| | | 4.06-3.91 (m, 1H), 3.32-3.06 (m, | | |
| | | 1H), 2.22-2.02 (m, 2H), 2.02-1.93 | | |
| | | (m, 1H), 1.84-1.49 (m, 3H). | | |
| 188 | 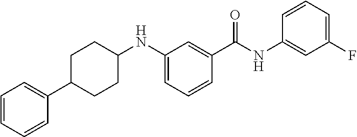 [SMILES: O=C(Nc1cccc(F)c1)c1cccc(NC2CCC(c3ccccc3)CC2)c1] | [Diastereomer A] ¹H NMR (400 MHz, DMSO-d₆) δ 10.29 (s, 1H), 7.77 (d, J = 11.9 Hz, 1H), 7.58 (d, J = 7.6 Hz, 1H), 7.44- 7.16 (m, 7H), 7.12-7.02 (m, 2H), 6.97-6.89 (m, 1H), 6.82 (d, J = 7.2 Hz, 1H), 5.81 (d, J = 7.9 Hz, 1H), 2.12 (d, J = 11.1 Hz, 2H), 1.87 (d, J = 12.2 Hz, 2H), 1.65 (q, J = 12.1 Hz, | 389 | Method E |
| | | 2H), 1.34 (q, J = 11.3 Hz, 2H). | | |
| | | [Diastereomer B] | | |
| | | ¹H NMR (400 MHz, DMSO-d₆) δ | | |
| | | 10.29 (s, 1H), 7.76 (d, J = 11.9 Hz, | | |
| | | 1H), 7.58 (d, J = 8.1 Hz, 1H), 7.40- | | |
| | | 7.15 (m, 8H), 7.08 (d, J = 7.3 Hz, 1H), | | |
| | | 6.97-6.85 (m, 2H), 5.99 (d, J = 7.8 | | |
| | | Hz, 1H), 3.76 (s, 1H), 1.97-1.65 (m, | | |
| | | 6H), 1.66-1.56 (m, 2H). | | |
| 189 | 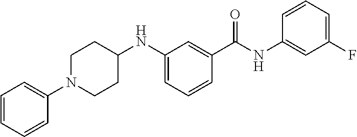 [SMILES: O=C(Nc1cccc(F)c1)c1cccc(NC2CCN(c3ccccc3)CC2)c1] | ¹H NMR (400 MHz, DMSO-d₆) δ 10.30 (s, 1H). 7.77 (d, J = 11.9 Hz, 1H), 7.58 (d, J = 8.1 Hz, 1H), 7.39 (q, J = 7.7 Hz, 1H), 7.28-7.17 (m, 3H), 7.15-7.04 (m, 2H), 7.01-6.81 (m, 4H), 6.77 (t, J = 7.1 Hz, 1H), 5.87 (d, J = 8.1 Hz, 1H), 3.70 (d, J = 12.5 Hz, 2H), 3.51 (s, 1H), 2.90 (t, J = 11.1 Hz, 2H), 2.02 (d, J = 11.4 Hz, 2H), 1.59- | 390 | Method E |
| | | 1.44 (m, 2H). | | |
| 190 | 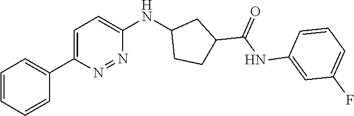 [SMILES: O=C(Nc1cccc(F)c1)C1CCC(Nc2ccc(-c3ccccc3)nn2)C1] | ¹H NMR (400 MHz, DMSO-d₆) δ 10.16 (s, 1H), 7.96 (d, J = 7.6 Hz, 2H), 7.80 (d, J = 9.4 Hz, 1H), 7.64 (d, J = 12.0 Hz, 1H), 7.47 (t, J = 7.3 Hz, 2H), 7.43-7.28 (m, 3H), 7.10 (d, J = 7.1 Hz, 1H), 6.92-6.81 (m, 2H), 4.39 (q, J = 7.2 Hz, 1H), 2.98-2.86 | 377 | Method E |
| | | (m, 1H), 2.14-2.03 (m, 1H), 1.95 (q, | | |
| | | J = 7.5 Hz, 2H), 1.73 (dd, J = 10.1, | | |
| | | 21.4 Hz, 1H), 1.68-1.58 (m, 1H). | | |
| 191 | 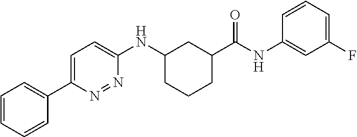 [SMILES: O=C(Nc1cccc(F)c1)C1CCCC(Nc2ccc(-c3ccccc3)nn2)C1] | ¹H NMR (400 MHz, DMSO-d₆) δ 10.19 (s, 1H), 7.98 (d, J = 7.3 Hz, 2H), 7.81 (d, J = 8.9 Hz, 1H), 7.63 (d, J = 11.7 Hz, 1H), 7.47 (t, J = 7.6 Hz, 2H), 7.43-7.28 (m, 3H), 6.95 (d, J = 7.6 Hz, 1H), 6.85 (d, J = 9.0 Hz, 2H), 4.07-3.94 (m, 1H), 2.23 (d, J = 12.6 Hz, 1H), 2.08 (d, J = 10.5 Hz, 1H), 1.93-1.80 (m, 2H), | 391 | Method E |
| | | 1.54-1.33 (m, 3H). 1.29-1.12 (m, 2H). | | |
| 192 | 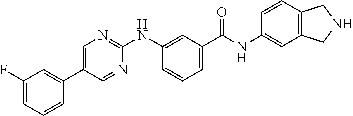 [SMILES: O=C(Nc1ccc2c(c1)CNC2)c1cccc(Nc2ncc(-c3cccc(F)c3)cn2)c1] | ¹H NMR (400 MHz, DMSO-d₆) δ 10.26-10.23 (m, 1H), 10.10-10.07 (m. 1H), 8.95-8.93 (m, 2H), 8.34 (s, 1H), 8.02-7.98 (m, 1H). 7.77 (s, 1H), 7.69-7.44 (m, 7H), 7.27-7.17 (m, 2H), 4.13 (d, J = 13.6 Hz, 2H). | 426 | Method E |
| 193 | 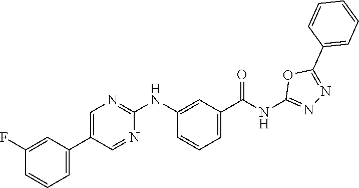 [SMILES: O=C(Nc1nnc(-c2ccccc2)o1)c1cccc(Nc2ncc(-c3cccc(F)c3)cn2)c1] | ¹H NMR (400 MHz, DMSO-d₆) δ 12.16 (s, 1H), 10.15 (s, 1H). 8.95 (s, 2H), 8.49 (s, 1H), 8.06 (dd, J = 1.4, 8.1 Hz, 1H), 8.02-7.98 (m, 2H), 7.68-7.60 (m, 6H), 7.57-7.48 (m, 2H), 7.21 (dt, J = 2.2, 8.6 Hz, 1H). | 453 | Method B |
| 194 | 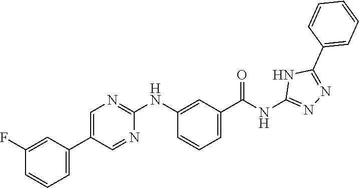 [SMILES: O=C(Nc1nnc(-c2ccccc2)[nH]1)c1cccc(Nc2ncc(-c3cccc(F)c3)cn2)c1] | ¹H NMR (400 MHz, DMSO-d₆) δ 10.22 (s, 1H), 8.92 (s, 2H), 8.68 (t, J = 1.9 Hz, 1H), 8.10 (dd, J = 1.3, 8.2 Hz, 1H), 8.02-7.98 (m, 2H), 7.88 (s, 2H), 7.81 (d, J = 7.9 Hz, 1H), 7.64 (td, J = 1.9, 10.4 Hz, 1H), 7.59 (d, J = 6.9 Hz. 1H), 7.56-7.45 (m, 5H), 7.24- 7.18 (m, 1H). | 452 | Method B |
| 195 | 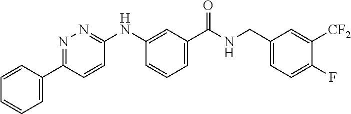 | ¹H NMR (400 MHz, DMSO-d₆) δ 9.59 (s, 1H), 9.12 (t, J = 5.9 Hz, 1H), 8.26 (s, 1H), 8.13-7.95 (m, 4H), 7.84-7.63 (m, 2H), 7.59-7.38 (m, 6H), 7.24 (d, J = 9.3 Hz, 1H), 4.54 (d, J = 5.8 Hz, 2H). | 467 | Method B |
| 196 | 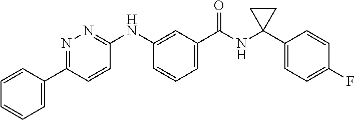 [SMILES: O=C(NC1(c2ccc(F)cc2)CC1)c1cccc(Nc2ccc(-c3ccccc3)nn2)c1] | ¹H NMR (400 MHz, DMSO-d₆) δ 9.56 (s, 1H), 9.19 (s, 1H), 8.18-8.17 (m, 1H), 8.13-7.94 (m, 4H), 7.57- 7.39 (m, 5H), 7.35-7.21 (m, 3H), 7.14-7.08 (m, 2H), 1.28-1.24 (m, 4H). | 425 | Method B |
| 197 | 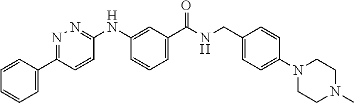 [SMILES: CN1CCN(c2ccc(CNC(=O)c3cccc(Nc4ccc(-c5ccccc5)nn4)c3)cc2)CC1] | ¹H NMR (400 MHz, CDCl₃) δ 8.02- 7.99 (m, 2H), 7.87 (s, 1H), 7.74- 7.69 (m, 2H), 7.52-7.41 (m, 5H), 7.31-7.27 (m, 2H), 7.20-7.16 (m, 1H), 7.03-6.99 (m, 1H), 6.94-6.90 (m, 2H), 6.41-6.38 (m, 1H), 4.57 (d, J = 5.7 Hz, 2H), 3.21 (t, J = 4.9 Hz, | 479 | Method B |
| | | 4H), 2.57 (t, J = 4.9 Hz, 4H), 2.35 (s, | | |
| | | 3H). | | |
| 198 | 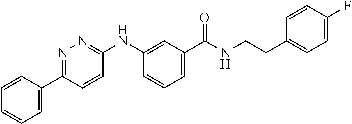 [SMILES: O=C(NCCc1ccc(F)cc1)c1cccc(Nc2ccc(-c3ccccc3)nn2)c1] | ¹H NMR (400 MHz, DMSO-d₆) δ 9.56 (s, 1H), 8.53 (t, J = 5.5 Hz, 1H), 8.21 (s, 1H), 8.11-8.02 (m, 3H), 7.99-7.95 (m, 1H), 7.58-7.35 (m, 5H), 7.35-7.21 (m, 3H), 7.16-7.10 (m, 2H), 3.52-3.40 (m, 2H), 2.85 (t, J = 7.3 Hz, 2H). | 413 | Method B |
| 199 | 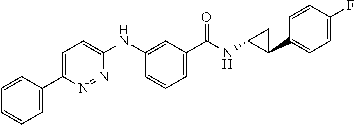 [SMILES: O=C(N[C@@H]1C[C@H]1c1ccc(F)cc1)c1cccc(Nc2ccc(-c3ccccc3)nn2)c1] | ¹H NMR (400 MHz, DMSO-d₆) δ 9.56 (s, 1H), 8.69 (d, J = 4.1 Hz, 1H), 8.19 (s, 1H), 8.12-7.95 (m, 4H), 7.58-7.36 (m, 5H), 7.36-7.17 (m, 3H), 7.17-6.99 (m, 2H), 3.04-2.97 (m, 1H), 2.14-2.07 (m, 1H), 1.39- 1.32 (m, 1H), 1.26-1.19 (m, 1H). | 425 | Method B |
| 199-1 | 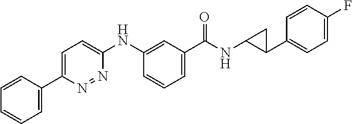 [SMILES: O=C(NC1CC1c1ccc(F)cc1)c1cccc(Nc2ccc(-c3ccccc3)nn2)c1] | — | — | — |
| 200 | 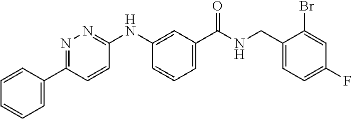 [SMILES: O=C(NCc1ccc(F)cc1Br)c1cccc(Nc2ccc(-c3ccccc3)nn2)c1] | ¹H NMR (400 MHz, DMSO-d₆) δ 9.60 (s, 1H), 9.06 (t, J = 5.8 Hz, 1H), 8.26 (s, 1H), 8.13-8.01 (m, 4H), 7.62 (dd, J = 2.6, 8.5 Hz, 1H), 7.56- 7.34 (m, 6H), 7.32-7.21 (m, 2H). 4.49 (d, J = 5.5 Hz, 2H). | 477 | Method B |
| 201 | 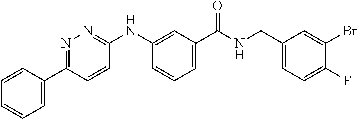 [SMILES: O=C(NCc1ccc(F)c(Br)c1)c1cccc(Nc2ccc(-c3ccccc3)nn2)c1] | ¹H NMR (400 MHz, DMSO-d₆) δ 9.58 (s, 1H), 9.08 (t, J = 6.0 Hz, 1H), 8.26-8.23 (m, 1H), 8.17-8.01 (m, 4H), 7.66 (dd, J = 1.9, 6.8 Hz, 1H), 7.58-7.31 (m, 7H), 7.24 (d, J = 9.4 Hz, 1H), 4.47(d, J = 5.8 Hz, 2H). | 477 | Method B |
| 202 | 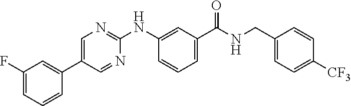 [SMILES: O=C(NCc1ccc(C(F)(F)F)cc1)c1cccc(Nc2ncc(-c3cccc(F)c3)cn2)c1] | ¹H NMR (400 MHz, DMSOd₆) δ- 10.01 (s, 1H), 9.11 (t, J = 5.9 Hz, 1H), 8.92 (s, 2H), 8.30 (t, J = 1.8 Hz, 1H), 7.97-7.94 (m, 1H), 7.71 (d, J = 8.1 Hz, 2H), 7.66-7.49 (m, 6H), 7.41 (t, J = 7.9 Hz, 1H), 7.21-7.20 (m, 1H), 4.56 (d, J = 5.8 Hz, 2H). | 467 | Method B |
| 203 | 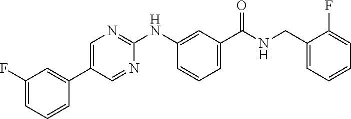 [SMILES: O=C(NCc1ccccc1F)c1cccc(Nc2ncc(-c3cccc(F)c3)cn2)c1] | ¹H NMR (400 MHz, DMSO-d₆) δ 10.01 (s, 1H), 8.99 (t, J = 5.8 Hz, 1H), 8.91 (s, 2H), 8.29 (t, J = 1.8 Hz, 1H), 7.97-7.94 (m, 1H), 7.63-7.59 (m, 2H), 7.52-7.49 (m, 2H), 7.43-7.39 (m, 2H), 7.37-7.30 (m, 1H), 7.22- 7.17 (m, 3H), 4.52 (d, J = 5.8 Hz, 2H). | 417 | Method B |
| 204 | 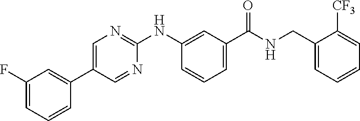 [SMILES: O=C(NCc1ccccc1C(F)(F)F)c1cccc(Nc2ncc(-c3cccc(F)c3)cn2)c1] | ¹H NMR (400 MHz, DMSO-d₆) δ 10.03 (s, 1H), 9.08 (t, J = 5.8 Hz, 1H), 8.91 (s, 2H), 8.33 (t, J = 1.8 Hz, 1H), 7.99 (dd, J = 8.0, 1.4 Hz, 1H), 7.75 (d, J = 7.8 Hz, 1H), 7.68-7.59 (m, 3H), 7.55-7.42 (m, 5H), 7.28-7.19 (m, 1H), 4.68 (d, J = 5.6 Hz, 2H). | 467 | Method B |
| 205 | 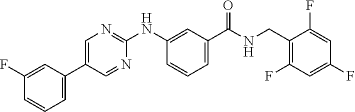 [SMILES: O=C(NCc1c(F)cc(F)cc1F)c1cccc(Nc2ncc(-c3cccc(F)c3)cn2)c1] | ¹H NMR (400 MHz, DMSO-d₆) δ 9.97 (s, 1H), 8.90 (s, 2H), 8.83 (t, J = 5.1 Hz, 1H), 8.23 (t, J = 1.8 Hz, 1H), 7.93-7.90 (m, 1H), 7.65-7.59 (m, 2H), 7.52-7.49 (m, 1H), 7.43- 7.37 (m, 2H), 7.21-7.17 (m, 3H), 4.47 (d, J = 5.0 Hz, 2H). | 453 | Method B |
| 206 | 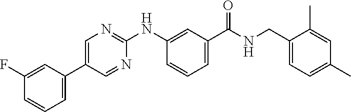 [SMILES: Cc1ccc(CNC(=O)c2cccc(Nc3ncc(-c4cccc(F)c4)cn3)c2)c(C)c1] | ¹H NMR (400 MHz, DMSO-d₆) δ 9.99 (s, 1H), 8.90 (s, 2H), 8.80 (t, J = 5.7 Hz, 1H), 8.26 (t, J = 1.8 Hz, 1H), 7.96-7.94 (m, 1H), 7.65-7.59 (m, 2H), 7.52-7.47 (m, 2H), 7.39 (t, J = 7.9 Hz, 1H), 7.23-7.19 (m, 1H), 7.14 (d, J = 7.7 Hz, 1H), 6.99-6.96 | 427 | Method B |
| | | (m, 2H), 4.41 (d, J = 5.7 Hz, 2H), 2.30 | | |
| | | (s, 3H), 2.25 (s, 3H). | | |
| 207 | 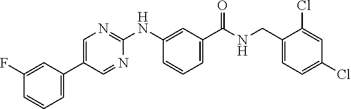 [SMILES: O=C(NCc1ccc(Cl)cc1Cl)c1cccc(Nc2ncc(-c3cccc(F)c3)cn2)c1] | ¹H NMR (400 MHz, DMSO-d₆) δ 10.02 (s, 1H), 9.03 (t, J = 5.6 Hz, 1H), 8.91 (s, 2H), 8.31-8.30 (m, 1H), 7.98-7.96 (m, 1H), 7.66-7.59 (m, 3H), 7.54-7.49 (m, 2H), 7.45-7.37 (m, 3H), 7.22-7.18 (m, 1H), 4.52 (d, J = 5.7 Hz, 2H). | 469 | Method B |
| 208 | 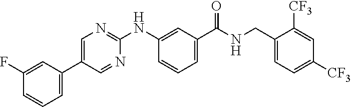 [SMILES: O=C(NCc1ccc(C(F)(F)F)cc1C(F)(F)F)c1cccc(Nc2ncc(-c3cccc(F)c3)cn2)c1] | ¹H NMR (400 MHz, DMSO-d₆) δ 10.05 (s, 1H), 9.02 (t, J = 5.6 Hz, 1H), 8.91 (s, 2H), 8.31-8.30 (m, 1H), 8.10 (d, J = 8.0 Hz, 1H), 8.04 (s, 1H), 7.99 (d, J = 8.0, 1.6 Hz, 1H), 7.78 (d, J = 8.0 Hz, 1H), 7.66-7.43 (m, 5H), 7.22-7.18 (m, 1H), 4.73 (d, J = 5.2 | 535 | Method B |
| | | Hz, 2H). | | |
| 209 | 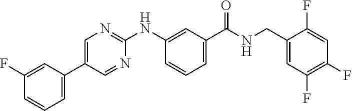 [SMILES: O=C(NCc1cc(F)c(F)cc1F)c1cccc(Nc2ncc(-c3cccc(F)c3)cn2)c1] | ¹H NMR (400 MHz, DMSO-d₆) δ 10.01 (s, 1H), 9.00 (t, J = 5.6 Hz, 1H), 8.91 (s, 2H), 8.28 (s, 1H), 7.97-7.94 (m, 1H), 7.66-7.39 (m, 7H), 7.21- 7.19 (m, 1H), 4.47 (d, J = 5.6 Hz, 2H). | 453 | Method B |
| 210 | 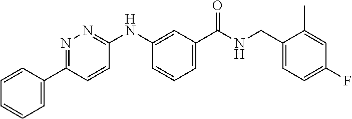 [SMILES: Cc1cc(F)ccc1CNC(=O)c1cccc(Nc2ccc(-c3ccccc3)nn2)c1] | ¹H NMR (400 MHz, DMSO-d₆) δ 9.58 (s, 1H), 8.91 (t, J = 5.7 Hz, 1H), 8.22 (t, J = 2.0 Hz, 1H), 8.13-7.93 (m, 4H), 7.57-7.39 (m, 5H), 7.34- 7.21 (m, 2H), 7.10-6.93 (m, 2H), 4.44 (d, J = 5.6 Hz, 2H), 2.36 (s, 3H). | 413 | Method B |
| 211 | 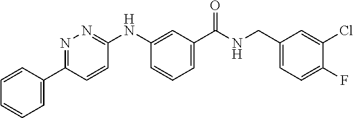 [SMILES: O=C(NCc1ccc(F)c(Cl)c1)c1cccc(Nc2ccc(-c3ccccc3)nn2)c1] | ¹H NMR (400 MHz, DMSO-d₆) δ 9.60 (s, 1H), 9.08 (t, J = 6.1 Hz, 1H), 8.25 (s, 1H), 8.13-7.95 (m, 4H), 7.60-7.30 (m, 8H), 7.25 (d, J = 9.4 Hz, 1H), 4.47 (d, J = 5.9 Hz, 2H). | 433 | Method B |
| 212 | 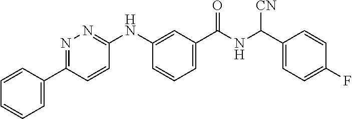 [SMILES: N#CC(NC(=O)c1cccc(Nc2ccc(-c3ccccc3)nn2)c1)c1ccc(F)cc1] | ¹H NMR (400 MHz, DMSO-d₆) δ 9.76 (d, J = 7.9 Hz, 1H), 9.61 (s, 1H), 8.28-8.26 (m, 1H), 8.10-8.04 (m, 4H), 7.65-7.59 (m, 2H), 7.54-7.44 (m, 5H), 7.35-7.23 (m, 3H), 6.44 (d, J = 7.8 Hz, 1H). | 424 | Method B |
| 213 | 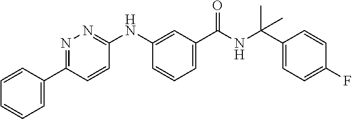 [SMILES: CC(C)(NC(=O)c1cccc(Nc2ccc(-c3ccccc3)nn2)c1)c1ccc(F)cc1] | ¹H NMR (400 MHz, DMSO-d₆) δ 9.54 (s, 1H), 8.45 (s, 1H), 8.11-8.01 (m, 5H), 7.57-7.36 (m, 7H), 7.24 (d, J = 9.3 Hz, 1H), 7.14-7.08 (m, 2H), 1.68 (s, 6H). | 427 | Method B |
| 214 | 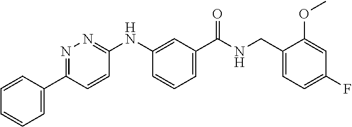 [SMILES: COc1cc(F)ccc1CNC(=O)c1cccc(Nc2ccc(-c3ccccc3)nn2)c1] | ¹H NMR (400 MHz, DMSO-d₆) δ 9.58 (s, 1H), 8.84 (t, J = 5.9 Hz, 1H), 8.24 (s, 1H), 8.13-7.98 (m, 4H), 7.58-7.38 (m, 5H), 7.28-7.15 (m, 2H), 6.92 (dd, J = 2.4, 11.3 Hz, 1H), 6.77-6.71 (m, 1H), 4.41 (d, J = 5.8 Hz, 2H), 3.85 (s, 3H). | 429 | Method B |
| 215 | 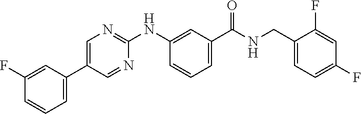 [SMILES: O=C(NCc1ccc(F)cc1F)c1cccc(Nc2ncc(-c3cccc(F)c3)cn2)c1] | ¹H NMR (400 MHz, DMSO-d₆) δ 10.00 (s, 1H), 8.98-8.91 (m, 3H), 8.29-8.27 (m, 1H), 7.95-7.93 (m, 1H), 7.65-7.42 (m, 6H), 7.21-7.08 (m, 3H), 4.49-4.47 (m, 2H). | 435 | Method B |
| 216 | 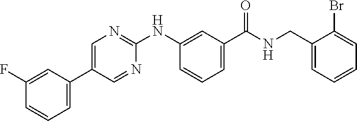 [SMILES: O=C(NCc1ccccc1Br)c1cccc(Nc2ncc(-c3cccc(F)c3)cn2)c1] | ¹H NMR (400 MHz, DMSO-d₆) δ 10.02 (s, 1H), 9.02 (t, J = 5.8 Hz, 1H), 8.91 (s, 2H), 8.31 (t, J = 1.8 Hz, 1H), 7.99-7.97 (m, 1H), 7.66-7.59 (m, 3H), 7.55-7.51 (m, 2H), 7.45-7.35 (m, 3H), 7.23-7.20 (m, 2H), 4.51 (d, J = 5.6 Hz, 2H). | 479 | Method B |
| 217 | 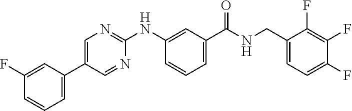 [SMILES: O=C(NCc1ccc(F)c(F)c1F)c1cccc(Nc2ncc(-c3cccc(F)c3)cn2)c1] | ¹H NMR (400 MHz, DMSO-d₆) δ 10.01 (s, 1H), 9.03 (t, J = 5.6 Hz, 1H), 8.91 (s, 2H), 8.29 (t, J = 1.8 Hz, 1H), 7.97-7.94 (m, 1H), 7.66-7.59 (m, 2H), 7.53-7.47 (m, 2H), 7.41 (t, J = 8.0 Hz, 1H), 7.25-7.20 (m, 3H), 4.51 (d, J = 5.6 Hz, 2H). | 453 | Method B |
| 218 | 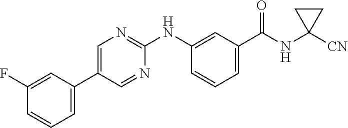 [SMILES: N#CC1(NC(=O)c2cccc(Nc3ncc(-c4cccc(F)c4)cn3)c2)CC1] | ¹H NMR (400 MHz, DMSO-d₆) δ 10.04 (s, 1H), 9.29 (s, 1H), 8.93 (s, 2H), 8.32-8.31 (m, 1H), 7.96-7.94 (m, 1H), 7.67-7.60 (m, 2H), 7.55- 7.51 (m, 1H), 7.42-7.40 (m, 2H), 7.22-7.20 (m, 1H), 1.59-1.55 (m, 2H), 1.30-1.27 (m, 2H). | 374 | Method B |
| 219 | 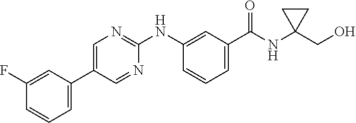 [SMILES: O=C(NC1(CO)CC1)c1cccc(Nc2ncc(-c3cccc(F)c3)cn2)c1] | ¹H NMR (400 MHz, DMSO-d₆) δ 9.96 (s, 1H), 8.91 (s, 2H), 8.59 (s, 1H), 8.23-8.21 (m, 1H), 7.93-7.91 (m, 1H), 7.66-7.59 (m, 2H), 7.55- 7.49 (m, 1H), 7.45-7.43 (m, 1H), 7.35 (t, J = 8.0 Hz, 1H), 7.22-7.20 (m, 1H), 4.77 (t, J = 5.6 Hz, 1H), 3.55 (d, J = 5.6 Hz, 2H), 0.79-0.75 (m, | 379 | Method B |
| | | 2H), 0.73-0.69 (m, 2H). | | |
| 220 | 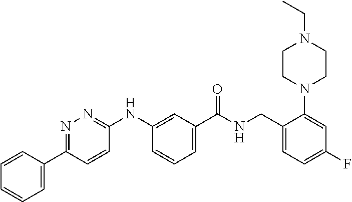 [SMILES: CCN1CCN(c2cc(F)ccc2CNC(=O)c2cccc(Nc3ccc(-c4ccccc4)nn3)c2)CC1] | ¹H NMR (400 MHz, DMSO-d₆) δ 9.58 (s, 1H), 8.92 (t, J = 5.8 Hz, 1H), 8.27 (t, J = 2.0 Hz, 1H), 8.11-8.00 (m, 4H), 7.63-7.39 (m, 5H), 7.33- 7.14 (m, 2H), 6.99-6.82 (m, 2H), 4.52 (d, J = 5.6 Hz, 2H), 2.91-2.89 (m, 4H), 2.63-2.51 (m, 4H), 2.40 (q, J = 7.2 Hz, 2H), 1.03 (t, J = 7.2 Hz, 3H). | 511 | Method D |
| 221 | 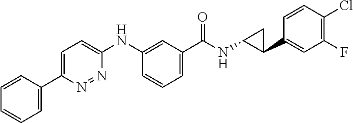 [SMILES: O=C(N[C@@H]1C[C@H]1c1ccc(Cl)c(F)c1)c1cccc(Nc2ccc(-c3ccccc3)nn2)c1] | ¹H NMR (400 MHz, DMSO-d₆) δ 9.56 (s, 1H), 8.72 (d, J = 4.3 Hz, 1H), 8.20 (s, 1H), 8.14-7.96 (m, 5H), 7.59-7.38 (m, 5H), 7.27-7.22 (m, 2H), 7.09 (dd, J = 1.8, 8.3 Hz, 1H), 3.11-3.04 (m, 1H), 2.16-2.10 (m, 1H), 1.47-1.41 (m, 1H), 1.35-1.28 (m, 1H). | 459 | Method B |
| 221-1 | 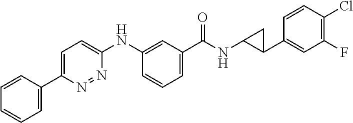 [SMILES: O=C(NC1CC1c1ccc(Cl)c(F)c1)c1cccc(Nc2ccc(-c3ccccc3)nn2)c1] | — | — | — |
| 222 | 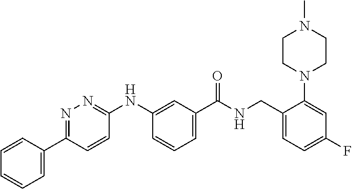 [SMILES: CN1CCN(c2cc(F)ccc2CNC(=O)c2cccc(Nc3ccc(-c4ccccc4)nn3)c2)CC1] | ¹H NMR (400 MHz, DMSO-d₆) δ 9.59 (s, 1H), 8.93 (t, J = 5.8 Hz, 1H), 8.27 (s, 1H), 8.08-8.02 (m, 4H), 7.55-7.41 (m, 5H), 7.31-7.23 (m, 2H), 6.98-6.86 (m, 2H), 4.52 (d, J = 5.9 Hz, 2H), 2.97-2.93 (m, 4H), 2.69-2.54 (m, 4H), 2.32 (s, 3H). | 497 | Method D |
| 223 | 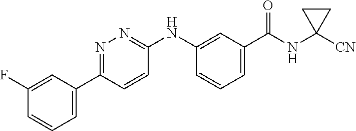 [SMILES: N#CC1(NC(=O)c2cccc(Nc3ccc(-c4cccc(F)c4)nn3)c2)CC1] | ¹H NMR (400 MHz, DMSO-d₆) δ 9.67 (s, 1H), 9.34 (s, 1H), 8.26-8.24 (m, 1H), 8.11 (d, J = 9.6 Hz, 1H), 8.06-8.04 (m, 1H), 7.94-7.90 (m, 2H), 7.58-7.54 (m, 1H), 7.48-7.41 (m, 2H), 7.31-7.29 (m, 1H), 7.25 (d, J = 9.6 Hz, 1H), 1.60-1.56 (m, 2H), 1.31-1.28 (m, 2H). | 374 | Method B |
| 224 | 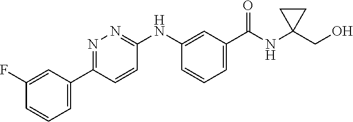 [SMILES: O=C(NC1(CO)CC1)c1cccc(Nc2ccc(-c3cccc(F)c3)nn2)c1] | ¹H NMR (400 MHz, DMSO-d₆) δ 9.60 (s, 1H), 8.65 (s, 1H), 8.19-8.17 (m, 1H), 8.09 (d, J = 9.2 Hz, 1H), 8.01-7.99 (m, 1H), 7.94-7.89 (m, 2H), 7.58-7.54 (m, 1H), 7.46 (d, J = 8.0 Hz, 1H), 7.39 (t, J = 8.0 Hz, 1H), 7.32-7.29 (m, 1H), 7.24 (d, J = 9.2 Hz, 1H), 4.78 (t, J = 5.6 Hz, 1H), 3.56 (d, | 379 | Method B |
| | | J = 5.6 Hz, 2H), 0.79-0.76 (m, 2H), | | |
| | | 0.73-0.69 (m, 2H). | | |
| 225 | 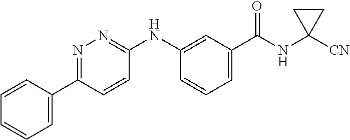 [SMILES: N#CC1(NC(=O)c2cccc(Nc3ccc(-c4ccccc4)nn3)c2)CC1] | ¹H NMR (400 MHz, DMSO-d₆) δ 9.61 (s, 1H), 9.33 (s, 1H), 8.26-8.24 (m, 1H), 8.09-8.05 (m, 4H), 7.54- 7.51 (m, 2H), 7.48-7.40 (m, 3H), 7.25 (d, J = 9.2 Hz, 1H), 1.60-1.56 (m, 2H), 1.31-1.28 (m, 2H). | 356 | Method B |
| 226 | 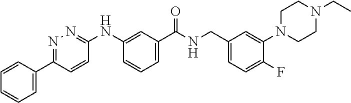 [SMILES: CCN1CCN(c2cc(CNC(=O)c3cccc(Nc4ccc(-c5ccccc5)nn4)c3)ccc2F)CC1] | ¹H NMR (400 MHz, DMSO-d₆) δ 9.58 (s, 1H), 9.00 (t, J = 6.8 Hz, 2H), 8.26 (s, 1H), 8.15-7.91 (m, 4H), 7.61-7.33 (m, 4H), 7.24 (d, J = 9.4 Hz, 1H), 7.16-6.97 (m, 2H), 6.94- 6.90 (m, 1H), 4.43 (d, J = 5.6 Hz, 2H), 3.41-3.36 (m, 4H), 3.01-2.97 (m, | 511 | Method D |
| | | 4H), 2.43-2.30 (m, 2H), 1.02 (t, | | |
| | | J = 6.9 Hz, 3H). | | |
| 227 | 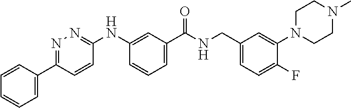 [SMILES: CN1CCN(c2cc(CNC(=O)c3cccc(Nc4ccc(-c5ccccc5)nn4)c3)ccc2F)CC1] | ¹H NMR (400 MHz, DMSO-d₆) δ 9.58 (s, 1H), 8.99 (t, J = 5.8 Hz, 1H), 8.25 (s, 1H), 8.15-7.93 (m, 4H), 7.61-7.36 (m, 5H), 7.25 (d, J = 9.4 Hz, 1H), 7.15-6.96 (m, 2H), 6.93- 6.89 (m, 1H), 4.43 (d, J = 5.6 Hz, 2H), 3.03-3.00 (m, 4H), 2.48-2.38 (m, | 497 | Method D |
| | | 4H), 2.20 (s, 3H). | | |
| 228 | 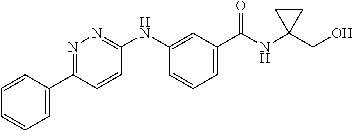 [SMILES: O=C(NC1(CO)CC1)c1cccc(Nc2ccc(-c3ccccc3)nn2)c1] | ¹H NMR (400 MHz, DMSO-d₆) δ 9.54 (s, 1H), 8.64 (s, 1H), 8.19-8.17 (m, 1H), 8.08-8.00 (m, 4H), 7.52 (t, J = 7.6 Hz, 2H), 7.45 (t, J = 7.6 Hz, 2H), 7.39 (d, J = 8.0 Hz, 1H), 7.24 (d, J = 9.2 Hz, 1H), 4.79 (t, J = 6.0 Hz, 1H), 3.56 (d, J = 6.0 Hz, 2H), 0.80- 0.76 (m, 2H), 0.73-0.69 (m, 2H). | 361 | Method B |
| 229 | 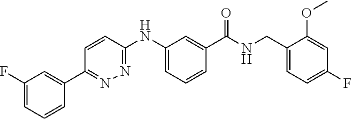 [SMILES: COc1cc(F)ccc1CNC(=O)c1cccc(Nc2ccc(-c3cccc(F)c3)nn2)c1] | ¹H NMR (400 MHz, DMSO-d₆) δ 9.67 (s, 1H), 8.84 (t, J = 5.9 Hz, 1H), 8.24 (t, J = 1.8 Hz, 1H), 8.10 (d, J = 9.4 Hz, 1H), 8.04-8.01 (m, 1H), 7.94- 7.88 (m, 2H), 7.60-7.51 (m, 2H), 7.45 (t, J = 7.9 Hz, 1H), 7.30-7.18 (m, 3H), 7.00-6.89 (m, 1H), 6.81- 6.71 (m, 1H), 4.41 (d, J = 5.8 Hz, | 447 | Method B |
| | | 2H), 3.85 (s, 3H). | | |
| 230 | 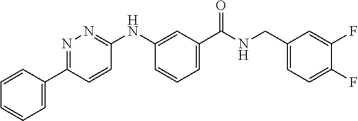 [SMILES: O=C(NCc1ccc(F)c(F)c1)c1cccc(Nc2ccc(-c3ccccc3)nn2)c1] | ¹H NMR (400 MHz, DMSO-d₆) δ 9.58 (s, 1H), 9.07 (t, J = 5.8 Hz, 1H), 8.25 (s, 1H), 8.13-7.94 (m, 4H), 7.59-7.33 (m, 7H), 7.32-6.93 (m, 2H), 4.47 (d, J = 5.9 Hz, 2H). | 417 | Method B |
| 231 | 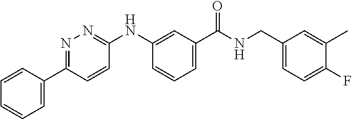 [SMILES: Cc1cc(CNC(=O)c2cccc(Nc3ccc(-c4ccccc4)nn3)c2)ccc1F] | ¹H NMR (400 MHz, DMSO-d₆) δ 9.58 (s, 1H), 9.01 (t, J = 5.9 Hz, 1H), 8.26-8.23 (m, 1H), 8.12-7.95 (m, 3H), 7.59-7.39 (m, 6H), 7.29-7.21 (m, 2H), 7.21-7.16 (m, 1H), 7.12- 7.06 (m, 1H), 4.43 (d, J = 5.9 Hz, 2H), 2.23 (d, J = 1.4 Hz, 3H). | 413 | Method B |
| 232 | 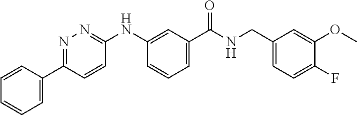 [SMILES: COc1cc(CNC(=O)c2cccc(Nc3ccc(-c4ccccc4)nn3)c2)ccc1F] | ¹H NMR (400 MHz, DMSO-d₆) δ 9.58 (s, 1H), 9.02 (t, J = 5.9 Hz, 1H), 8.26 (s, 1H), 8.13-7.94 (m, 4H), 7.60-7.38 (m, 5H), 7.31-7.07 (m, 3H), 6.92-6.87 (m, 1H), 4.46 (d, J = 5.8 Hz, 2H), 3.83 (s, 3H). | 429 | Method B |
| 233 | 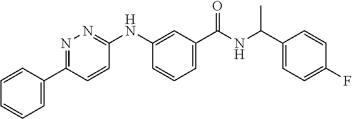 [SMILES: CC(NC(=O)c1cccc(Nc2ccc(-c3ccccc3)nn2)c1)c1ccc(F)cc1] | ¹H NMR (400 MHz, DMSO-d₆) δ 9.57 (s, 1H), 8.81 (d, J = 8.1 Hz, 1H), 8.16-8.12 (m, 1H), 8.12-8.01 (m, 4H), 7.59-7.38 (m, 7H), 7.30-7.10 (m, 3H), 5.23-5.15 (m, 1H), 1.48 (d, J = 7.0 Hz, 3H). | 413 | Method B |
| 234 | 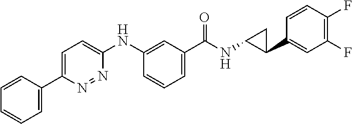 [SMILES: O=C(N[C@@H]1C[C@H]1c1ccc(F)c(F)c1)c1cccc(Nc2ccc(-c3ccccc3)nn2)c1] | ¹H NMR (400 MHz, DMSO-d₆) δ 9.58 (s, 1H), 8.71 (d, J = 4.3 Hz, 1H), 8.22-8.18 (m, 1H), 8.11-1.96 (m, 4H), 7.62-7.40 (m, 5H), 7.40-7.20 (m, 3H), 7.08-7.04 (m, 1H), 3.07- 3.00 (m, 1H), 2.14-2.08 (m, 1H), 1.43-1.36 (m, 1H), 1.31-1.24 (m, 1H). | 443 | Method B |
| 234-1 | 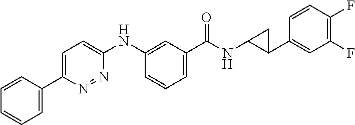 [SMILES: O=C(NC1CC1c1ccc(F)c(F)c1)c1cccc(Nc2ccc(-c3ccccc3)nn2)c1] | — | — | — |
| 235 | 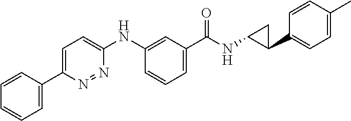 [SMILES: Cc1ccc([C@@H]2C[C@H]2NC(=O)c2cccc(Nc3ccc(-c4ccccc4)nn3)c2)cc1] | ¹H NMR (400 MHz, DMSO-d₆) δ 9.58 (s, 1H), 8.67 (d, J = 4.4 Hz, 1H), 8.18 (s, 1H), 8.15-7.94 (m, 4H), 7.56-7.49 (m, 2H), 7.49-7.37 (m, 3H), 7.24 (d, J = 9.3 Hz, 1H), 7.15- 7.01 (m, 4H), 3.04-2.98 (m, 1H), 2.27 (s, 3H), 2.09-2.02 (m, 1H), 1.36-1.29 (m, 1H), 1.22-1.15 (m, | 421 | Method B |
| | | 1H). | | |
| 235-1 | 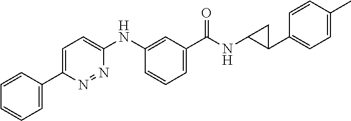 [SMILES: Cc1ccc(C2CC2NC(=O)c2cccc(Nc3ccc(-c4ccccc4)nn3)c2)cc1] | — | — | — |
| 236 | 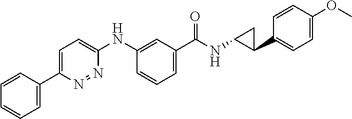 [SMILES: COc1ccc([C@@H]2C[C@H]2NC(=O)c2cccc(Nc3ccc(-c4ccccc4)nn3)c2)cc1] | ¹H NMR (400 MHz, DMSO-d₆) δ 9.57 (s, 1H), 8.66 (d, J = 4.3 Hz, 1H), 8.18 (s, 1H), 8.14-7.95 (m, 4H), 7.52 (dd, J = 7.4, 7.4 Hz, 2H), 7.49- 7.39 (m, 3H), 7.24 (d, J = 9.4 Hz, 1H), 7.12 (d, J = 8.6 Hz, 2H), 6.86 (d, J = 8.6 Hz, 2H), 3.73 (s, 3H), 3.00- | 437 | Method B |
| | | 2.93 (m, 1H), 2.08-2.02 (m, 1H), | | |
| | | 1.33-1.26 (m, 1H), 1.19-1.12 (m, | | |
| | | 1H). | | |
| 236-1 | 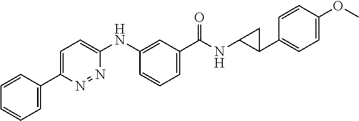 [SMILES: COc1ccc(C2CC2NC(=O)c2cccc(Nc3ccc(-c4ccccc4)nn3)c2)cc1] | — | — | — |
| 237 | 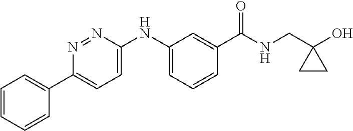 [SMILES: O=C(NCC1(O)CC1)c1cccc(Nc2ccc(-c3ccccc3)nn2)c1] | ¹H NMR (400 MHz, DMSO-d₆) δ 9.56 (s, 1H), 8.38-8.36 (m, 1H), 8.20-8.19 (m, 1H), 8.08-8.03 (m, 4H), 7.54-7.50 (m, 2H), 7.48-7.41 (m, 3H), 7.24 (d, J = 9.2 Hz, 1H), 3.45 (d, J = 6.0 Hz, 2H), 0.60-0.54 (m, 4H). | 361 | Method B |

Table 2 lists the chemical structures, catalog numbers, and purchase source of the compounds that were used for the assay described in the following examples.

TABLE 2
Compound Structure, Catalog Number, and Purchase Source

| | | Cat. No. or | |
|---|---|---|---|
| | Structure | CAS No. | Source |
| A1 | 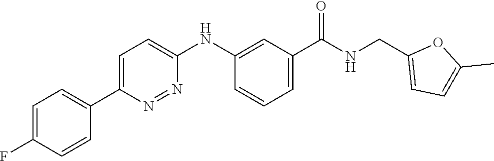 [SMILES: Cc1ccc(CNC(=O)c2cccc(Nc3ccc(-c4ccc(F)cc4)nn3)c2)o1] | F575-0555F | Chemdiv, Inc |
| A2 | 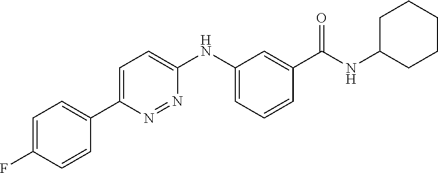 [SMILES: O=C(NC1CCCCC1)c1cccc(Nc2ccc(-c3ccc(F)cc3)nn2)c1] | F575-0112 | ChemDiv, Inc. |
| A3 | 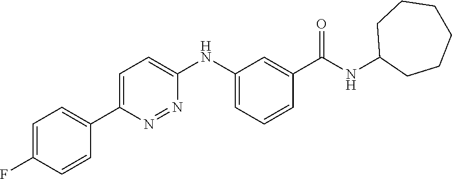 [SMILES: O=C(NC1CCCCCC1)c1cccc(Nc2ccc(-c3ccc(F)cc3)nn2)c1] | F575-0118 | ChemDiv, Inc. |
| A4 | 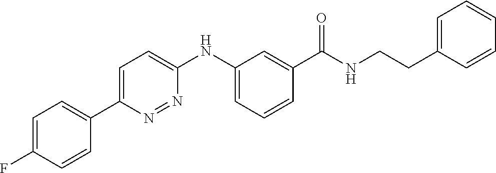 [SMILES: O=C(NCCc1ccccc1)c1cccc(Nc2ccc(-c3ccc(F)cc3)nn2)c1] | F575-0181 | ChemDiv, Inc. |
| A5 | 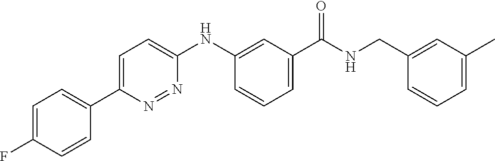 [SMILES: Cc1cccc(CNC(=O)c2cccc(Nc3ccc(-c4ccc(F)cc4)nn3)c2)c1] | F575-0188 | ChemDiv, Inc. |
| A6 | 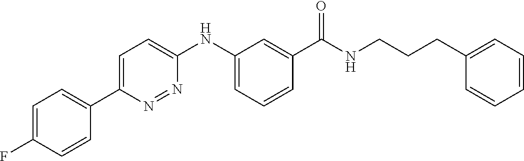 [SMILES: O=C(NCCCc1ccccc1)c1cccc(Nc2ccc(-c3ccc(F)cc3)nn2)c1] | F575-0190 | ChemDiv, Inc |
| A7 | 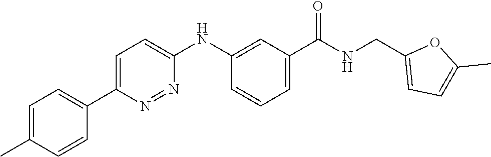 | F575-0605 | ChemDiv, Inc. |
| A8 | 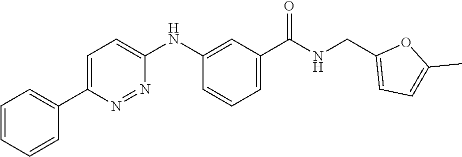 [SMILES: Cc1ccc(CNC(=O)c2cccc(Nc3ccc(-c4ccccc4)nn3)c2)o1] | F575-0705 | ChemDiv, Inc. |
| A9 | 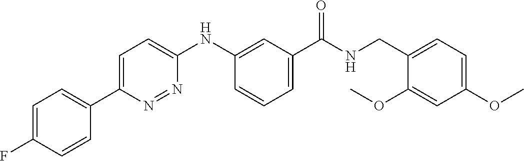 [SMILES: COc1ccc(CNC(=O)c2cccc(Nc3ccc(-c4ccc(F)cc4)nn3)c2)c(OC)c1] | F575-0193 | ChemDiv, Inc. |
| A10 | 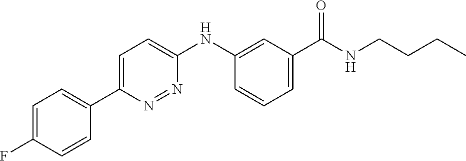 [SMILES: CCCCNC(=O)c1cccc(Nc2ccc(-c3ccc(F)cc3)nn2)c1] | F575-0100 | ChemDiv, Inc. |
| A11 | 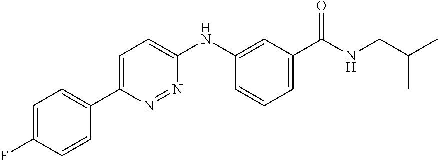 [SMILES: CC(C)CNC(=O)c1cccc(Nc2ccc(-c3ccc(F)cc3)nn2)c1] | F575-0102 | ChemDiv, Inc. |
| A12 | 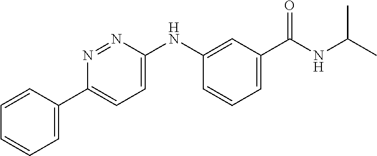 [SMILES: CC(C)NC(=O)c1cccc(Nc2ccc(-c3ccccc3)nn2)c1] | F575-0403 | ChemDiv, Inc. |
| A13 | 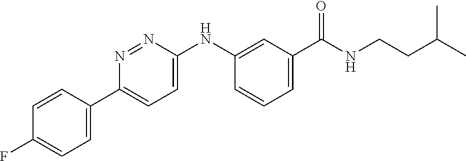 [SMILES: CC(C)CCNC(=O)c1cccc(Nc2ccc(-c3ccc(F)cc3)nn2)c1] | F575-0111 | ChemDiv, Inc. |
| A14 | 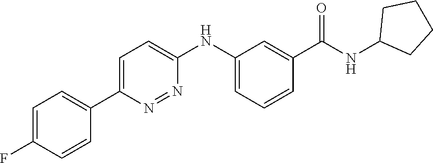 [SMILES: O=C(NC1CCCC1)c1cccc(Nc2ccc(-c3ccc(F)cc3)nn2)c1] | F575-0115 | ChemDiv, Inc. |
| A15 | 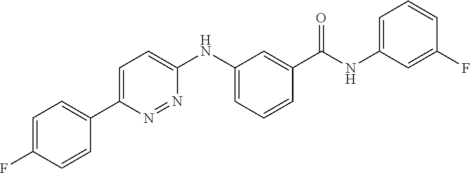 [SMILES: O=C(Nc1cccc(F)c1)c1cccc(Nc2ccc(-c3ccc(F)cc3)nn2)c1] | F575-0134 | ChemDiv, Inc. |
| A16 | 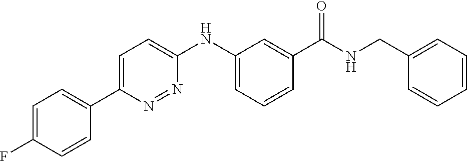 [SMILES: O=C(NCc1ccccc1)c1cccc(Nc2ccc(-c3ccc(F)cc3)nn2)c1] | F575-0186 | ChemDiv, Inc. |
| A17 | 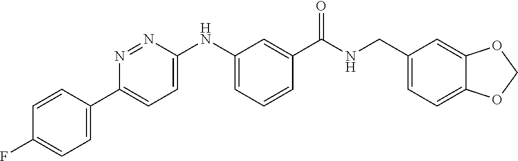 [SMILES: O=C(NCc1ccc2c(c1)OCO2)c1cccc(Nc2ccc(-c3ccc(F)cc3)nn2)c1] | F575-0191 | ChemDiv, Inc. |
| A18 | 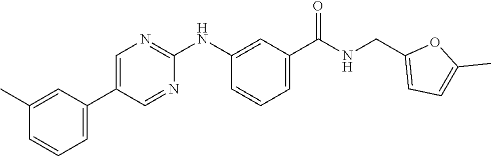 | L369-0053 | ChemDiv, Inc. |
| A19 | 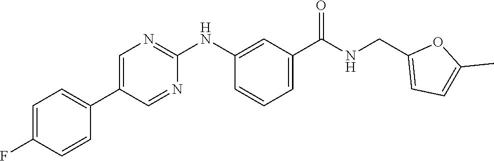 [SMILES: Cc1ccc(CNC(=O)c2cccc(Nc3ncc(-c4ccc(F)cc4)cn3)c2)o1] | L369-0203 | ChemDiv, Inc. |
| A20 | 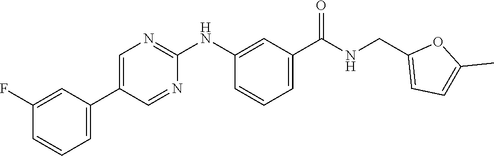 [SMILES: Cc1ccc(CNC(=O)c2cccc(Nc3ncc(-c4cccc(F)c4)cn3)c2)o1] | L369-0503 | ChemDiv, Inc. |
| A21 | 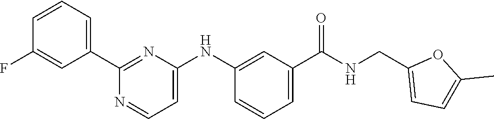 [SMILES: Cc1ccc(CNC(=O)c2cccc(Nc3ccnc(-c4cccc(F)c4)n3)c2)o1] | F871-0982 | ChemDiv, Inc. |
| A22 | 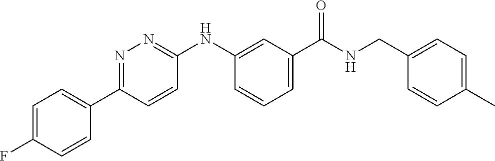 [SMILES: Cc1ccc(CNC(=O)c2cccc(Nc3ccc(-c4ccc(F)cc4)nn3)c2)cc1] | F575-0192 | ChemDiv, Inc. |
| A23 | 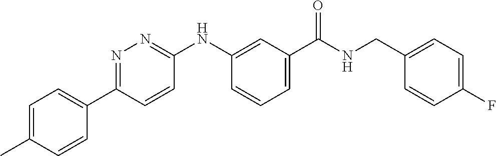 [SMILES: Cc1ccc(-c2ccc(Nc3cccc(C(=O)NCc4ccc(F)cc4)c3)nn2)cc1] | F575-0283 | ChemDiv, Inc. |
| A24 | 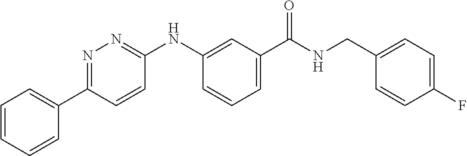 [SMILES: O=C(NCc1ccc(F)cc1)c1cccc(Nc2ccc(-c3ccccc3)nn2)c1] | F575-0482 | ChemDiv, Inc. |
| A25 | 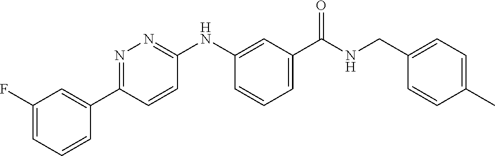 [SMILES: Cc1ccc(CNC(=O)c2cccc(Nc3ccc(-c4cccc(F)c4)nn3)c2)cc1] | F575-0391 | ChemDiv, Inc. |
| A26 | 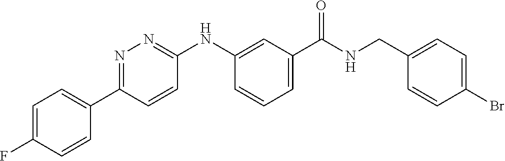 [SMILES: O=C(NCc1ccc(Br)cc1)c1cccc(Nc2ccc(-c3ccc(F)cc3)nn2)c1] | F575-0174 | ChemDiv, Inc. |
| A27 | 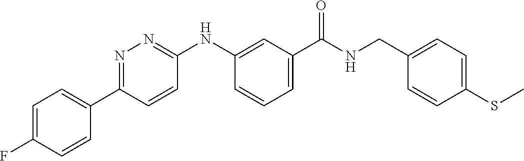 [SMILES: CSc1ccc(CNC(=O)c2cccc(Nc3ccc(-c4ccc(F)cc4)nn3)c2)cc1] | F575-0178 | ChemDiv, Inc. |
| A28 | 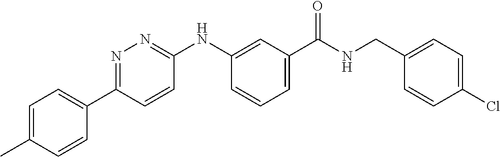 [SMILES: Cc1ccc(-c2ccc(Nc3cccc(C(=O)NCc4ccc(Cl)cc4)c3)nn2)cc1] | F575-0287 | ChemDiv, Inc. |
| A29 | 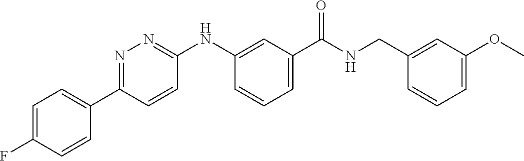 [SMILES: COc1cccc(CNC(=O)c2cccc(Nc3ccc(-c4ccc(F)cc4)nn3)c2)c1] | F575-0196 | ChemDiv, Inc. |
| A30 | 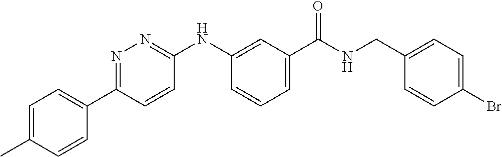 [SMILES: Cc1ccc(-c2ccc(Nc3cccc(C(=O)NCc4ccc(Br)cc4)c3)nn2)cc1] | F575-0274 | ChemDiv, Inc. |
| A31 | 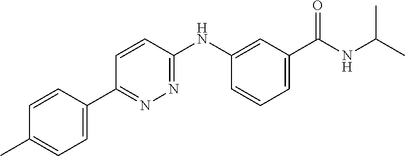 [SMILES: Cc1ccc(-c2ccc(Nc3cccc(C(=O)NC(C)C)c3)nn2)cc1] | F575-0204 | ChemDiv, Inc. |
| A32 | 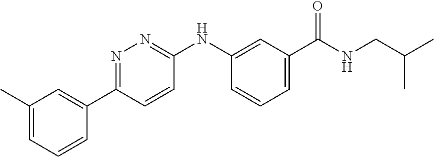 [SMILES: Cc1cccc(-c2ccc(Nc3cccc(C(=O)NCC(C)C)c3)nn2)c1] | F575-0003 | ChemDiv, Inc. |
| A33 | 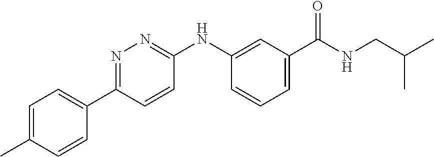 [SMILES: Cc1ccc(-c2ccc(Nc3cccc(C(=O)NCC(C)C)c3)nn2)cc1] | F575-0202 | ChemDiv, Inc. |
| A34 | 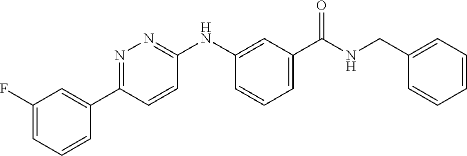 [SMILES: O=C(NCc1ccccc1)c1cccc(Nc2ccc(-c3cccc(F)c3)nn2)c1] | F575-0385 | ChemDiv, Inc. |
| A35 | 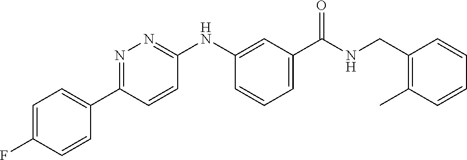 [SMILES: Cc1ccccc1CNC(=O)c1cccc(Nc2ccc(-c3ccc(F)cc3)nn2)c1] | F575-0185 | ChemDiv, Inc. |
| A36 | 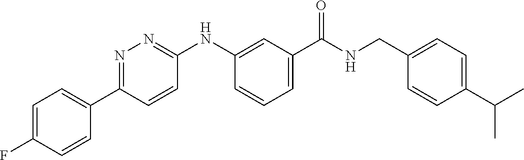 [SMILES: CC(C)c1ccc(CNC(=O)c2cccc(Nc3ccc(-c4ccc(F)cc4)nn3)c2)cc1] | F575-0194 | ChemDiv, Inc. |
| A37 | 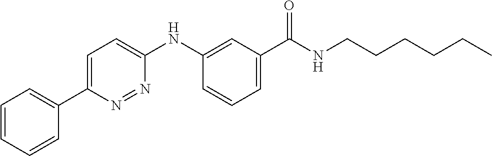 [SMILES: CCCCCCNC(=O)c1cccc(Nc2ccc(-c3ccccc3)nn2)c1] | F575-0400 | ChemDiv, Inc. |
| A38 | 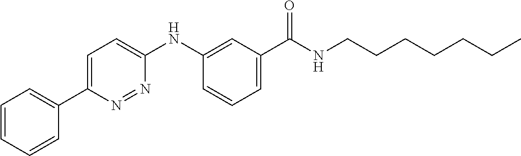 [SMILES: CCCCCCCNC(=O)c1cccc(Nc2ccc(-c3ccccc3)nn2)c1] | F575-0409 | ChemDiv, Inc. |
| A39 | 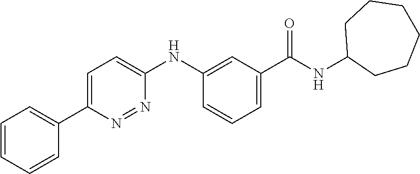 [SMILES: O=C(NC1CCCCCC1)c1cccc(Nc2ccc(-c3ccccc3)nn2)c1] | F575-0417 | ChemDiv, Inc. |
| A40 | 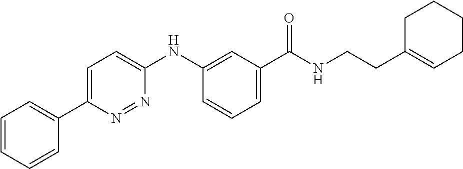 [SMILES: O=C(NCCC1=CCCCC1)c1cccc(Nc2ccc(-c3ccccc3)nn2)c1] | F575-0420 | ChemDiv, Inc. |
| A41 | 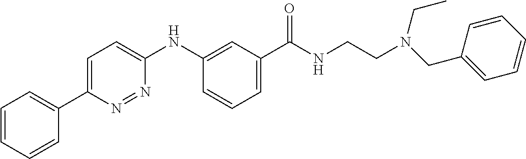 [SMILES: CCN(CCNC(=O)c1cccc(Nc2ccc(-c3ccccc3)nn2)c1)Cc1ccccc1] | F575-0462 | ChemDiv, Inc. |
| A42 | 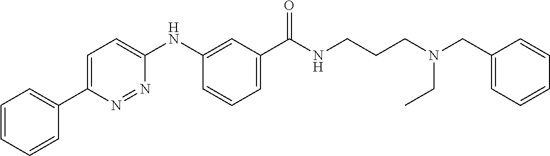 [SMILES: CCN(CCCNC(=O)c1cccc(Nc2ccc(-c3ccccc3)nn2)c1)Cc1ccccc1] | F575-0468 | ChemDiv, Inc. |
| A43 | 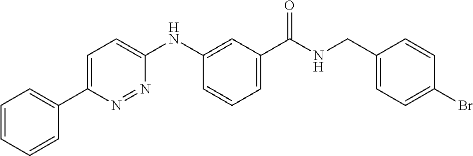 [SMILES: O=C(NCc1ccc(Br)cc1)c1cccc(Nc2ccc(-c3ccccc3)nn2)c1] | F575-0473 | ChemDiv, Inc. |
| A44 | 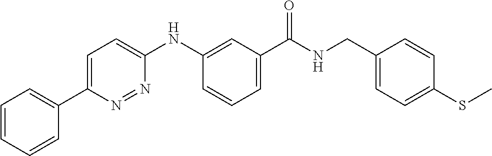 [SMILES: CSc1ccc(CNC(=O)c2cccc(Nc3ccc(-c4ccccc4)nn3)c2)cc1] | F575-0477 | ChemDiv, Inc. |
| A45 | 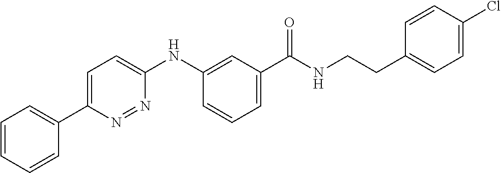 [SMILES: O=C(NCCc1ccc(Cl)cc1)c1cccc(Nc2ccc(-c3ccccc3)nn2)c1] | F575-0479 | ChemDiv, Inc. |
| A46 | 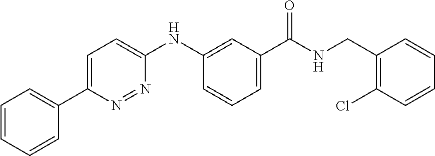 [SMILES: O=C(NCc1ccccc1Cl)c1cccc(Nc2ccc(-c3ccccc3)nn2)c1] | F575-0481 | ChemDiv, Inc. |
| A47 | 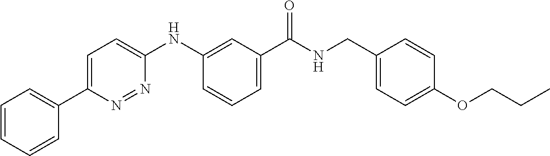 [SMILES: CCCOc1ccc(CNC(=O)c2cccc(Nc3ccc(-c4ccccc4)nn3)c2)cc1] | F575-0483 | ChemDiv, Inc. |
| A48 | 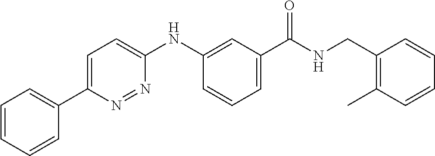 [SMILES: Cc1ccccc1CNC(=O)c1cccc(Nc2ccc(-c3ccccc3)nn2)c1] | F575-0484 | ChemDiv, Inc. |
| A49 | 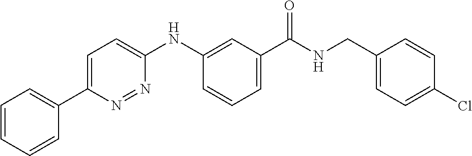 [SMILES: O=C(NCc1ccc(Cl)cc1)c1cccc(Nc2ccc(-c3ccccc3)nn2)c1] | F575-0486 | ChemDiv, Inc. |
| A50 | 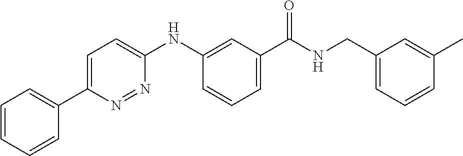 [SMILES: Cc1cccc(CNC(=O)c2cccc(Nc3ccc(-c4ccccc4)nn3)c2)c1] | F575-0487 | ChemDiv, Inc. |
| A51 | 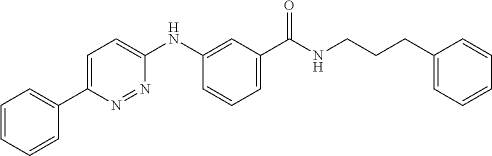 [SMILES: O=C(NCCCc1ccccc1)c1cccc(Nc2ccc(-c3ccccc3)nn2)c1] | F575-0489 | ChemDiv, Inc. |
| A52 | 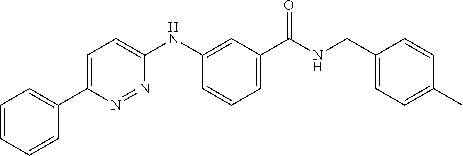 [SMILES: Cc1ccc(CNC(=O)c2cccc(Nc3ccc(-c4ccccc4)nn3)c2)cc1] | F575-0491 | ChemDiv, Inc. |
| A53 | 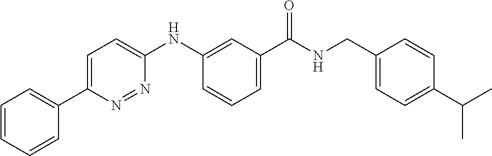 [SMILES: CC(C)c1ccc(CNC(=O)c2cccc(Nc3ccc(-c4ccccc4)nn3)c2)cc1] | F575-0493 | ChemDiv, Inc. |
| A54 | 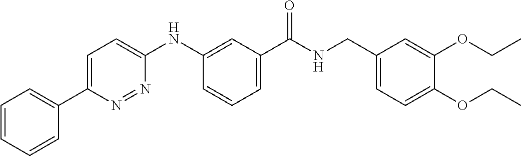 [SMILES: CCOc1ccc(CNC(=O)c2cccc(Nc3ccc(-c4ccccc4)nn3)c2)cc1OCC] | F575-0494 | ChemDiv, Inc. |
| A55 | 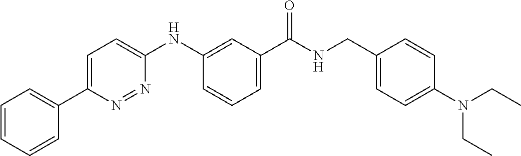 [SMILES: CCN(CC)c1ccc(CNC(=O)c2cccc(Nc3ccc(-c4ccccc4)nn3)c2)cc1] | F575-0498 | ChemDiv, Inc. |
| A56 | 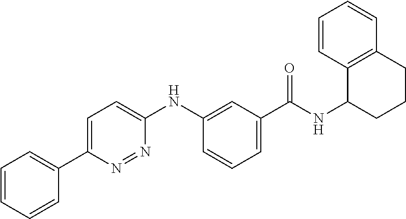 [SMILES: O=C(NC1CCCc2ccccc21)c1cccc(Nc2ccc(-c3ccccc3)nn2)c1] | F575-0708 | ChemDiv, Inc. |
| A57 | 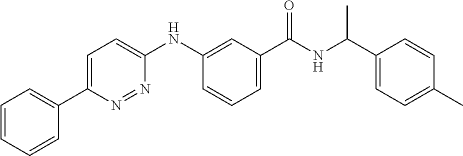 [SMILES: Cc1ccc(C(C)NC(=O)c2cccc(Nc3ccc(-c4ccccc4)nn3)c2)cc1] | F575-0710 | ChemDiv, Inc. |
| A58 | 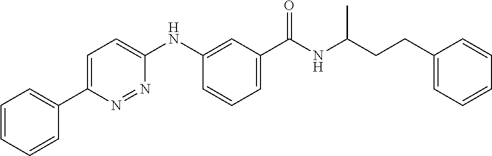 [SMILES: CC(CCc1ccccc1)NC(=O)c1cccc(Nc2ccc(-c3ccccc3)nn2)c1] | F575-0711 | ChemDiv, Inc. |
| A59 | 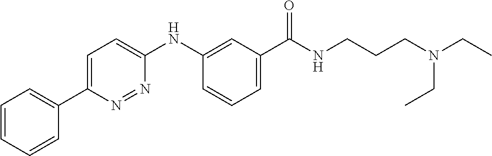 [SMILES: CCN(CC)CCCNC(=O)c1cccc(Nc2ccc(-c3ccccc3)nn2)c1] | F575-0419 | ChemDiv, Inc. |
| A60 | 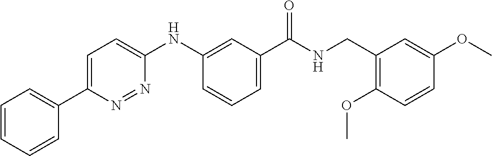 [SMILES: COc1ccc(OC)c(CNC(=O)c2cccc(Nc3ccc(-c4ccccc4)nn3)c2)c1] | F575-0478 | ChemDiv, Inc. |
| A61 | 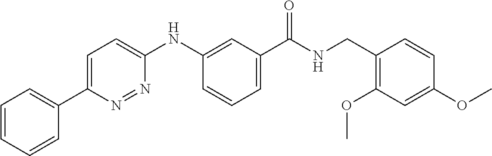 [SMILES: COc1ccc(CNC(=O)c2cccc(Nc3ccc(-c4ccccc4)nn3)c2)c(OC)c1] | F575-0492 | ChemDiv, Inc. |
| A62 | 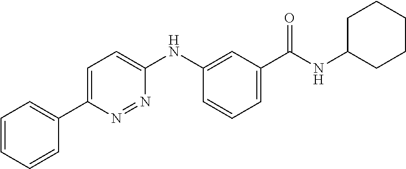 [SMILES: O=C(NC1CCCCC1)c1cccc(Nc2ccc(-c3ccccc3)nn2)c1] | F575-0411 | ChemDiv, Inc. |
| A63 | 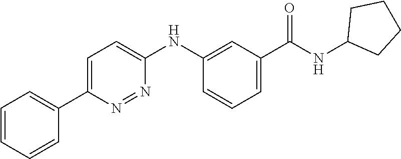 [SMILES: O=C(NC1CCCC1)c1cccc(Nc2ccc(-c3ccccc3)nn2)c1] | F575-0414 | ChemDiv, Inc. |
| A64 | 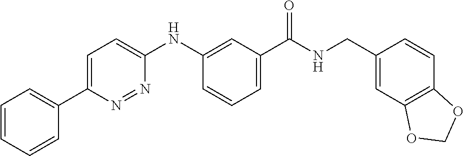 [SMILES: O=C(NCc1ccc2c(c1)OCO2)c1cccc(Nc2ccc(-c3ccccc3)nn2)c1] | F575-0490 | ChemDiv, Inc. |
| A65 | 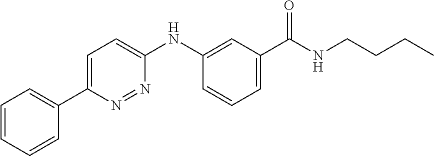 [SMILES: CCCCNC(=O)c1cccc(Nc2ccc(-c3ccccc3)nn2)c1] | F575-0399 | ChemDiv, Inc. |
| A66 | 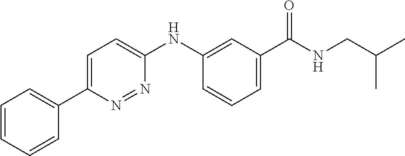 [SMILES: CC(C)CNC(=O)c1cccc(Nc2ccc(-c3ccccc3)nn2)c1] | F575-0401 | ChemDiv, Inc. |
| A67 | 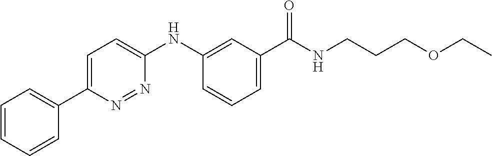 [SMILES: CCOCCCNC(=O)c1cccc(Nc2ccc(-c3ccccc3)nn2)c1] | F575-0404 | ChemDiv, Inc. |
| A68 | 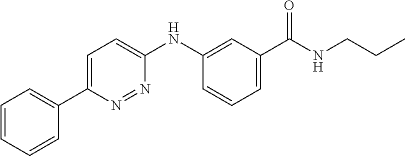 [SMILES: CCCNC(=O)c1cccc(Nc2ccc(-c3ccccc3)nn2)c1] | F575-0405 | ChemDiv, Inc. |
| A69 | 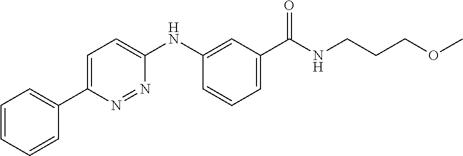 [SMILES: COCCCNC(=O)c1cccc(Nc2ccc(-c3ccccc3)nn2)c1] | F575-0406 | ChemDiv, Inc. |
| A70 | 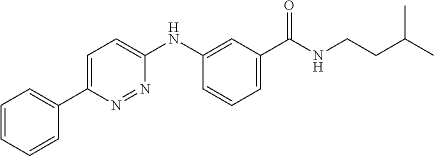 [SMILES: CC(C)CCNC(=O)c1cccc(Nc2ccc(-c3ccccc3)nn2)c1] | F575-0410 | ChemDiv, Inc. |
| A71 | 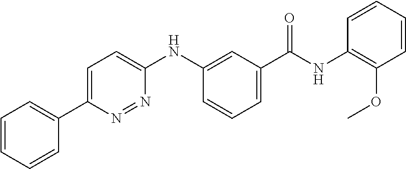 [SMILES: COc1ccccc1NC(=O)c1cccc(Nc2ccc(-c3ccccc3)nn2)c1] | F575-0442 | ChemDiv, Inc. |
| A72 | 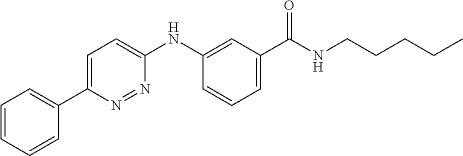 [SMILES: CCCCCNC(=O)c1cccc(Nc2ccc(-c3ccccc3)nn2)c1] | F575-0444 | ChemDiv, Inc. |
| A73 | 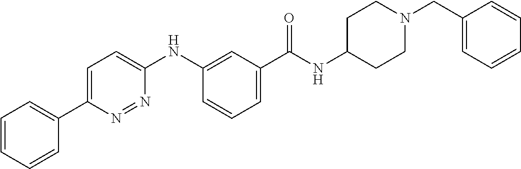 [SMILES: O=C(NC1CCN(Cc2ccccc2)CC1)c1cccc(Nc2ccc(-c3ccccc3)nn2)c1] | F575-0448 | ChemDiv, Inc. |
| A74 | 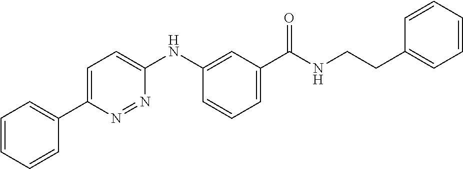 [SMILES: O=C(NCCc1ccccc1)c1cccc(Nc2ccc(-c3ccccc3)nn2)c1] | F575-0480 | ChemDiv, Inc. |
| A75 | 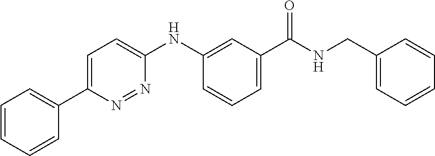 [SMILES: O=C(NCc1ccccc1)c1cccc(Nc2ccc(-c3ccccc3)nn2)c1] | F575-0485 | ChemDiv, Inc. |
| A76 | 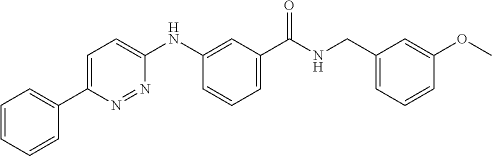 [SMILES: COc1cccc(CNC(=O)c2cccc(Nc3ccc(-c4ccccc4)nn3)c2)c1] | F575-0495 | ChemDiv, Inc. |
| A77 | 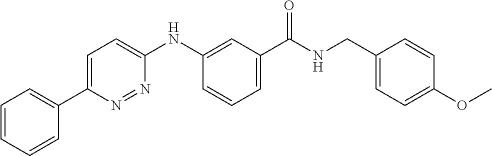 [SMILES: COc1ccc(CNC(=O)c2cccc(Nc3ccc(-c4ccccc4)nn3)c2)cc1] | F575-0496 | ChemDiv, Inc. |
| A78 | 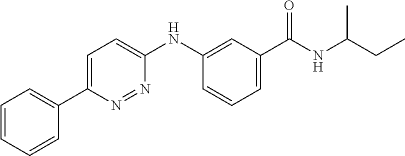 [SMILES: CCC(C)NC(=O)c1cccc(Nc2ccc(-c3ccccc3)nn2)c1] | F575-0699 | ChemDiv, Inc. |
| A79 | 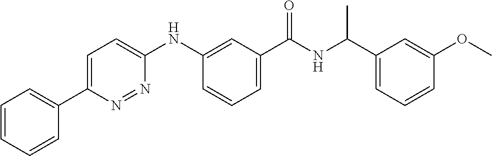 [SMILES: COc1cccc(C(C)NC(=O)c2cccc(Nc3ccc(-c4ccccc4)nn3)c2)c1] | F575-0709 | ChemDiv, Inc. |
| A80 | 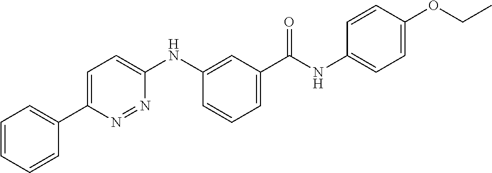 [SMILES: CCOc1ccc(NC(=O)c2cccc(Nc3ccc(-c4ccccc4)nn3)c2)cc1] | F575-0436 | ChemDiv, Inc. |
| A81 | 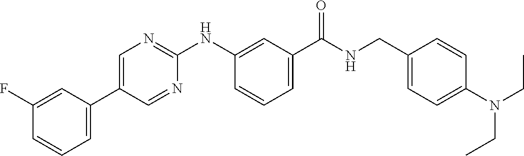 [SMILES: CCN(CC)c1ccc(CNC(=O)c2cccc(Nc3ncc(-c4cccc(F)c4)cn3)c2)cc1] | L369-0550 | ChemDiv, Inc. |
| A82 | 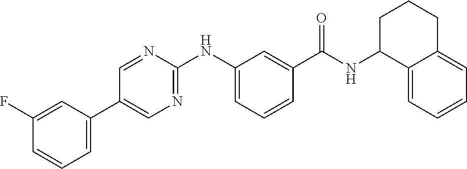 [SMILES: O=C(NC1CCCc2ccccc21)c1cccc(Nc2ncc(-c3cccc(F)c3)cn2)c1] | L369-0511 | ChemDiv, Inc. |
| A83 | 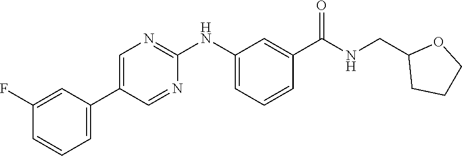 [SMILES: O=C(NCC1CCCO1)c1cccc(Nc2ncc(-c3cccc(F)c3)cn2)c1] | L369-0460 | ChemDiv, Inc. |
| A84 | 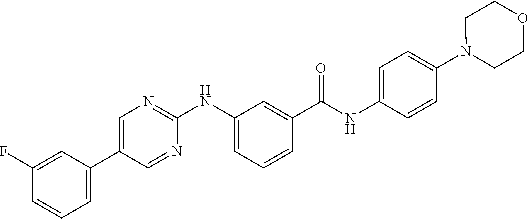 [SMILES: O=C(Nc1ccc(N2CCOCC2)cc1)c1cccc(Nc2ncc(-c3cccc(F)c3)cn2)c1] | L369-0495 | ChemDiv, Inc. |
| A85 | 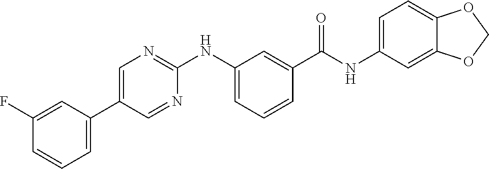 [SMILES: O=C(Nc1ccc2c(c1)OCO2)c1cccc(Nc2ncc(-c3cccc(F)c3)cn2)c1] | L369-0480 | ChemDiv, Inc. |
| A86 | 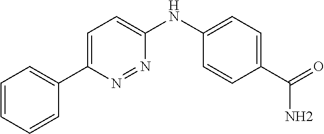 [SMILES: NC(=O)c1ccc(Nc2ccc(-c3ccccc3)nn2)cc1] | A47.142.586 | Aurora Fine chemicals |
| A87 | 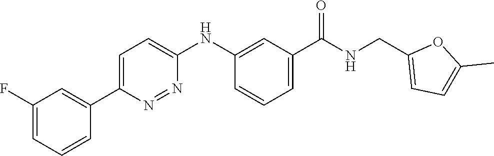 [SMILES: Cc1ccc(CNC(=O)c2cccc(Nc3ccc(-c4cccc(F)c4)nn3)c2)o1] | A34.266.199 | Aurora Fine chemicals |
| A88 | 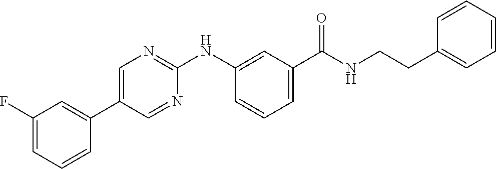 [SMILES: O=C(NCCc1ccccc1)c1cccc(Nc2ncc(-c3cccc(F)c3)cn2)c1] | L369-0532 | ChemDiv, Inc. |
| A89 | 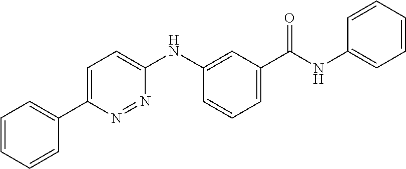 [SMILES: O=C(Nc1ccccc1)c1cccc(Nc2ccc(-c3ccccc3)nn2)c1] | CAS NO. 1111227-32-7 | — |
| A90 | 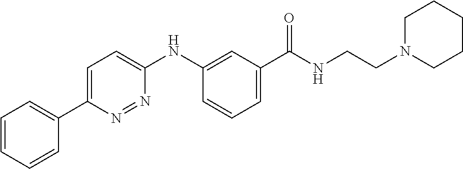 [SMILES: O=C(NCCN1CCCCC1)c1cccc(Nc2ccc(-c3ccccc3)nn2)c1] | A33.491.793 | Aurora Fine chemicals |
| A91 | 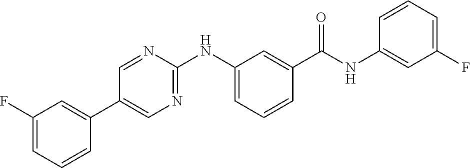 [SMILES: O=C(Nc1cccc(F)c1)c1cccc(Nc2ncc(-c3cccc(F)c3)cn2)c1] | CAS NO. 1224017-30-4 | — |
| A92 | 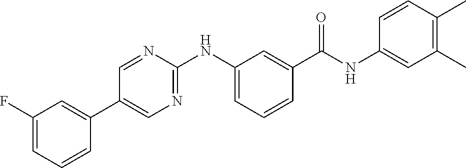 [SMILES: Cc1ccc(NC(=O)c2cccc(Nc3ncc(-c4cccc(F)c4)cn3)c2)cc1C] | CAS NO. 1223832-63-0 | — |
| A93 | 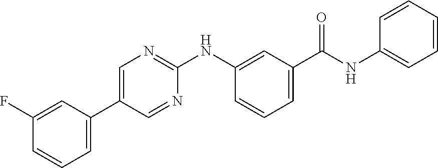 [SMILES: O=C(Nc1ccccc1)c1cccc(Nc2ncc(-c3cccc(F)c3)cn2)c1] | CAS NO. 1189643-31-9 | — |
| A94 | 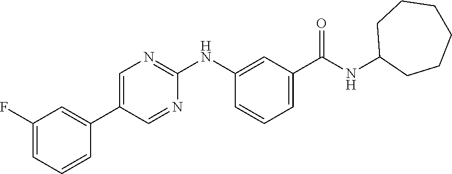 [SMILES: O=C(NC1CCCCCC1)c1cccc(Nc2ncc(-c3cccc(F)c3)cn2)c1] | CAS NO. 1223969-64-9 | — |
| A95 | 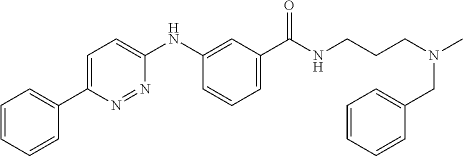 [SMILES: CN(CCCNC(=O)c1cccc(Nc2ccc(-c3ccccc3)nn2)c1)Cc1ccccc1] | CAS NO. 1111227-33-8 | — |
| A96 | 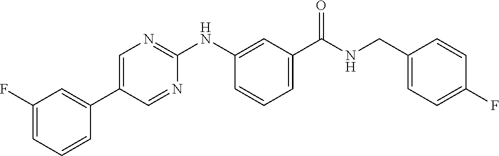 [SMILES: O=C(NCc1ccc(F)cc1)c1cccc(Nc2ncc(-c3cccc(F)c3)cn2)c1] | CAS NO. 1243048-89-6 | — |
| A97 | 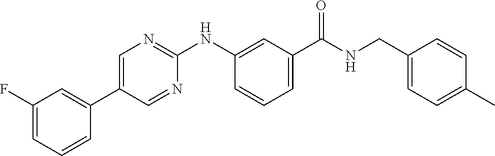 [SMILES: Cc1ccc(CNC(=O)c2cccc(Nc3ncc(-c4cccc(F)c4)cn3)c2)cc1] | CAS NO. 1242903-26-9 | — |
| A98 | 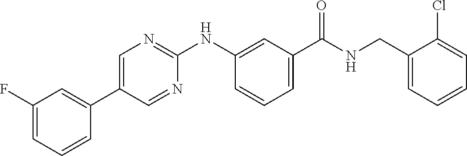 [SMILES: O=C(NCc1ccccc1Cl)c1cccc(Nc2ncc(-c3cccc(F)c3)cn2)c1] | CAS NO. 1242859-88-6 | — |
| A99 | 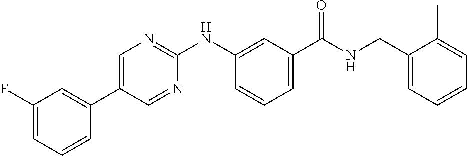 [SMILES: Cc1ccccc1CNC(=O)c1cccc(Nc2ncc(-c3cccc(F)c3)cn2)c1] | CAS NO. 1243020-99-6 | — |
| A100 | 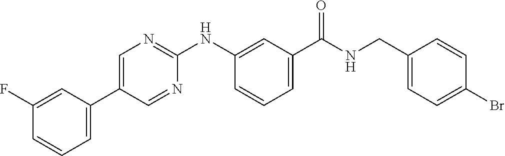 [SMILES: O=C(NCc1ccc(Br)cc1)c1cccc(Nc2ncc(-c3cccc(F)c3)cn2)c1] | CAS NO. 1185046-78-9 | — |
| A101 | 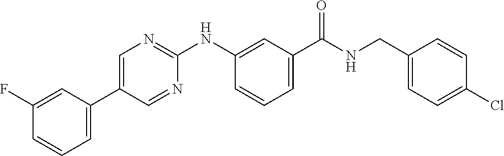 [SMILES: O=C(NCc1ccc(Cl)cc1)c1cccc(Nc2ncc(-c3cccc(F)c3)cn2)c1] | CAS NO. 1242957-31-8 | — |
| A102 | 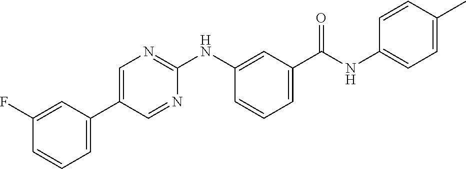 [SMILES: Cc1ccc(NC(=O)c2cccc(Nc3ncc(-c4cccc(F)c4)cn3)c2)cc1] | CAS NO. 1242902-58-4 | — |

### Example 3: YFP Quenching Assay

1. Materials and Instruments

Ionomycin (Alomonelab cat. #I-700), FLUOstar Omega microplate reader (BMG Labtech, Ortenberg, Germany), and MARS Data Analysis Software (BMG Labtech)

2. Cell Culture

Fisher rat thyroid (FRT) cells stably expressing human ANO6 (GenBank accession no. NP_001191732.1, provided by J. H. Nam, Dongguk University College of Medicine, Korea) and halide sensor mutant YFP-H148Q/I152L/F46L) were constructed and grown in Dulbecco's modified Eagle's medium Nutrient Mixture F-12 (DMEM/F-12) supplemented 10% FBS, 100 units/mL penicillin, 500 μg/mL hygromycin B and 100 μg/mL neomycin.

3. Assay Procedure

Fisher rat thyroid (FRT) cells stably expressing human ANO6 and halide sensor mutant YFP (H148Q/I152L/F46L) were seeded in black walled 96 well plates and incubated in a 37° C., 5% CO2 incubator to reach about 100% cell confluency. Then, each well of the 96 well plates were washed for several times with phosphate buffered saline (PBS), and 50 μL of PBS was added to each well. Test compounds (100× in DMSO) were added to each well to be 1% v/v DMSO. After incubation for 10 minutes in 40° C., the 96 well plates were transferred to a plate reader, and YFP fluorescence changed by SCN-introduced into cells through activated ANO6 were measured by the following steps.

(1) YFP fluorescence signals were recorded in every 0.4 seconds.

(2) Basal YFP fluorescence signals were recorded for 1 second.

(3) 140 mM SCN-(50 μL) containing 10 μM ionomycin was injected to each well, and YFP fluorescence signals were recorded.

The inhibitory activity (%) of each of the test compounds was obtained by the following steps.

(1) Background signals were subtracted from recording values, and the resulting values were converted to relative percentages. The values at 0 second were set to be 100%.

(2) Differences between the values at 3.6 seconds and those of at 7.6 seconds were calculated.

(3) In each row of 96 well plate, the inhibitory activity of each of the test compounds was calculated as percentages. The inhibitory activity of a negative control group to which neither compounds nor ionomycin were treated was set to be 100%, and the inhibitory activity of a positive control group to which ionomycin was treated and compounds were not treated was set to be 0%.

(4) The assay was performed in duplicate or triplicate, and the results were averaged.

The assay result is shown in Table 3, where ‘A’ means that the compound showed 60% or more inhibitory activities at each concentration (A≥60%), ‘B’ means that the compound showed inhibitory activities of 30% or more to less than 60% (60%>B≥30%) at each concentration, and ‘C’ means that the compound shows less than 30% inhibitory activities (30%>C) at each concentration.

TABLE 3
YFP Quenching Assay Result

| | Inhibition Level | Inhibition Level |
|---|---|---|
| Cmpd No. | 30 μM | 1 μM |
| 1 | A | B |
| 2 | A | B |
| 3 | A | A |
| 4 | A | A |
| 5 | B | — |
| 6 | A | A |
| 7 | A | A |
| 8 | A | B |
| 9 | A | C |
| 10 | A | A |
| 11 | A | B |
| 12 | B | B |
| 13 | A | C |
| 15 | B | C |
| 16 | C | — |
| 17 | B | — |
| 18 | B | C |
| 19 | A | C |
| 20 | A | B |
| 21 | C | C |
| 22 | C | — |
| 23 | A | C |
| 24 | A | B |
| 25 | A | B |
| 26 | A | B |
| 27 | A | B |
| 28 | A | C |
| 29 | A | A |
| 30 | A | A |
| 31 | A | A |
| 32 | A | A |
| 33 | A | A |
| 34 | A | C |
| 35 | A | C |
| 36 | A | A |
| 37 | A | B |
| 38 | A | A |
| 39 | A | A |
| 40 | A | A |
| 41 | A | B |
| 42 | C | — |
| 43 | — | C |
| 44 | — | A |
| 45 | — | A |
| 46 | — | B |
| 47 | — | C |
| 48 | — | C |
| 49 | — | C |
| 50 | — | A |
| 51 | — | A |
| 52 | — | A |
| 53 | — | A |
| 54 | — | A |
| 55 | — | A |
| 57 | — | A |
| 58 | — | A |
| 60 | — | A |
| 61 | — | C |
| 62 | — | C |
| 63 | — | B |
| 65 | — | B |
| 66 | — | B |
| 67 | — | A |
| 68 | — | A |
| 69 | — | C |
| 70 | — | C |
| 71 | — | C |
| 72 | — | C |
| 73 | — | A |
| 74 | — | A |
| 75 | — | B |
| 76 | — | C |
| 77 | — | C |
| 78 | — | B |
| 79 | — | A |
| 80 | — | A |
| 81 | — | A |
| 82 | — | B |
| 83 | — | B |
| 84 | — | C |
| 85 | — | B |
| 86 | — | C |
| 87 | — | C |
| 88 | — | C |
| 89 | — | C |
| 90 | — | A |
| 92 | — | C |
| 93 | — | C |
| 94 | — | C |
| 95 | — | C |
| 96 | — | C |
| 97 | — | C |
| 98 | — | A |
| 99 | — | B |
| 100 | — | C |
| 101 | — | C |
| 102 | — | C |
| 103 | — | C |
| 104 | — | C |
| 105 | — | C |
| 106 | — | B |
| 107 | — | C |
| 108 | — | C |
| 109 | — | C |
| 110 | — | B |
| 111 | — | C |
| 112 | — | A |
| 113 | — | C |
| 114 | — | A |
| 115 | — | A |
| 116 | — | A |
| 117 | — | A |
| 118 | — | A |
| 119 | — | A |
| 120 | — | C |
| 121 | — | A |
| 122 | — | A |
| 123 | — | A |
| 124 | — | B |
| 125 | — | A |
| A1 | A | B |
| A2 | A | A |
| A3 | A | A |
| A4 | A | A |
| A5 | A | A |
| A6 | A | A |
| A7 | A | C |
| A8 | A | A |
| A9 | B | — |
| A10 | A | A |
| A11 | A | A |
| A12 | A | A |
| A13 | A | A |
| A14 | A | A |
| A15 | A | A |
| A16 | A | B |
| A17 | A | B |
| A18 | B | B |
| A19 | B | — |
| A20 | B | C |
| A21 | C | — |
| A22 | A | A |
| A23 | B | — |
| A24 | A | A |
| A25 | A | B |
| A26 | A | B |
| A27 | A | B |
| A28 | B | — |
| A29 | A | B |
| A30 | C | — |
| A31 | A | B |
| A32 | A | A |
| A33 | A | B |
| A34 | A | A |
| A35 | A | A |
| A36 | A | A |
| A37 | A | A |
| A38 | A | A |
| A39 | A | B |
| A40 | A | A |
| A41 | A | B |
| A42 | A | B |
| A43 | A | A |
| A44 | A | B |
| A45 | A | A |
| A46 | A | B |
| A47 | A | A |
| A48 | A | A |
| A49 | A | B |
| A50 | A | A |
| A51 | A | A |
| A52 | A | A |
| A53 | A | A |
| A54 | A | B |
| A55 | A | A |
| A56 | A | B |
| A57 | A | A |
| A58 | A | A |
| A59 | B | C |
| A60 | A | B |
| A61 | A | B |
| A62 | A | B |
| A63 | A | A |
| A64 | A | B |
| A65 | A | A |
| A66 | A | A |
| A67 | A | A |
| A68 | A | A |
| A69 | A | B |
| A70 | A | A |
| A71 | B | — |
| A72 | A | A |
| A73 | A | A |
| A74 | A | A |
| A75 | A | B |
| A76 | A | A |
| A77 | A | A |
| A78 | A | A |
| A79 | A | B |
| A80 | — | A |
| A81 | — | B |
| A82 | — | B |
| A83 | — | B |
| A84 | — | A |
| A85 | — | A |
| A86 | B | — |
| A87 | — | A |
| A88 | — | B |
| A89 | — | A |
| A90 | — | C |

“—”: not tested

### Example 4: LACT C2 Assay

### Phosphatidylserine Scramblase Function Assay

1. Materials and Instruments

Ionomycin (Alomonelab, cat. #I-700), DAPI (Sigma-Aldrich, cat. #D8417), paraformaldehyde (Biosesang, cat. #P2031), Lionheart FX Automated Microscope (BioTek, Winooski), Python3 (Python Software Foundation), and OpenCV (Open Source Computer Vision Library).

2. Cell Culture

Fisher rat thyroid (FRT) cells stably expressing human ANO6 (GenBank accession no. NP_001191732.1, provided by J. H. Nam, Dongguk University College of Medicine, Korea) were grown in DMEM/Ham's F-12 (1:1) medium with 10% FBS, 2 mM L-549 glutamine, 100 units/mL penicillin, and 100 μg/mL streptomycin at 37° C. and 5% CO₂.

3. Assay Procedure

FRT cells stably expressing human ANO6 were plated in 96 well black-walled microplates at a density of 2×10⁴ cells/well. After 24 hours incubation, cells were treated with the test compound (dissolved in DMSO) were treated to each well to become 1% v/v DMSO for 10 minutes, then 10 μM of ionomycin was applied. Finally each well was washed with 200 μL PBS after 10 minutes. After washout, the phosphatidylserine and nuclei were stained with PBS containing 500 nM Lactadherin-C₂ (Lact-C₂)-GFP, then cells were washed with 200 μL PBS. Cells were fixed with 4% paraformaldehyde for 5 minutes at room temperature. For morphological analysis, some cells were stained with fluorescently labelled DAPI for 15 minutes at room temperature. Quantitative analysis of the fluorescence intensity of Lact-C₂-GFP was performed with Python3 and OpenCV. OpenCV was used to remove background and noise pixels, and the sum of all remaining pixel values was used to evaluate the fluorescence intensity of Lact-C₂-GFP.

The inhibitory activity (%) of each of the test compounds was obtained by the following steps. In each row of 96 well plate, the inhibitory activity of each of the test compounds was calculated as percentages. The inhibitory activity of a negative control group to which neither compounds nor ionomycin were treated was set to be 100%, and the inhibitory activity of a positive control group to which ionomycin was treated and compounds were not treated was set to be 0%. The assay was performed in triplicate, and the results were averaged.

The assay result is shown in Table 4, where ‘A’ means that the compound showed 60% or more inhibitory activities at each concentration (A≥60%), ‘B’ means that the compound showed inhibitory activities of 30% or more to less than 60% (60%>B≥30%) at each concentration, and ‘C’ means that the compound showed less than 30% inhibitory activities (30%>C) at each concentration.

TABLE 4
LACT C2 Assay Result

| | Inhibition Level | Inhibition Level |
|---|---|---|
| Cmpd No. | 1 μM | 100 nM |
| 1 | A | — |
| 2 | A | C |
| 3 | A | C |
| 4 | A | C |
| 6 | B | — |
| 7 | A | C |
| 10 | C | — |
| 24 | C | — |
| 25 | B | — |
| 29 | A | B |
| 30 | B | — |
| 31 | A | C |
| 32 | C | — |
| 33 | A | C |
| 36 | A | B |
| 38 | C | — |
| 39 | C | — |
| 40 | A | C |
| 41 | C | — |
| 44 | C | — |
| 45 | C | — |
| 46 | B | — |
| 50 | B | — |
| 51 | A | B |
| 52 | B | — |
| 53 | A | — |
| 54 | C | — |
| 57 | A | C |
| 58 | C | — |
| 60 | A | B |
| 63 | C | — |
| 66 | C | — |
| 67 | A | A |
| 68 | A | A |
| 73 | A | C |
| 74 | A | B |
| 75 | B | — |
| 78 | A | B |
| 79 | A | A |
| 80 | A | A |
| 81 | A | B |
| 82 | C | — |
| 90 | B | — |
| 92 | C | — |
| 98 | B | — |
| 105 | C | C |
| 110 | B | — |
| 112 | B | — |
| 114 | A | C |
| 115 | A | A |
| 116 | A | C |
| 117 | C | C |
| 118 | A | A |
| 119 | A | A |
| 120 | C | C |
| 121 | B | — |
| 122 | A | A |
| 123 | A | A |
| 125 | A | C |
| 126 | C | — |
| 127 | C | C |
| 128 | A | A |
| 129 | C | C |
| 130 | C | C |
| 131 | A | C |
| 132 | C | C |
| 133 | B | C |
| 134 | B | C |
| 135 | B | C |
| 136 | A | C |
| 137 | A | C |
| 138 | A | C |
| 139 | C | C |
| 140 | C | C |
| 141 | A | A |
| 142 | A | A |
| 143 | B | C |
| 144 | C | C |
| 145 | C | C |
| 146 | B | C |
| 147 | C | C |
| 148 | C | C |
| 149 | A | C |
| 150 | C | C |
| 151 | C | C |
| 152 | C | C |
| 153 | C | C |
| 154 | A | A |
| 155 | A | A |
| 156 | B | C |
| 157 | A | B |
| 158 | C | C |
| 159 | B | C |
| 160 | A | A |
| 161 | A | A |
| 162 | A | C |
| 163 | A | C |
| 164 | — | B |
| 165 | — | C |
| 166 | — | C |
| 167 | — | C |
| 168 | — | C |
| 170 | — | B |
| 171 | — | C |
| 172 | — | A |
| 173 | — | C |
| 174 | — | A |
| 175 | — | A |
| 177 | — | C |
| 178 | — | B |
| 179 | — | A |
| 180 | — | C |
| 181 | — | C |
| 187 | — | C |
| 188 | — | C |
| 191 | — | C |
| 192 | — | C |
| 194 | — | C |
| 195 | A | C |
| 196 | B | C |
| 197 | C | C |
| 198 | A | C |
| 199 | A | A |
| 200 | A | C |
| 201 | B | C |
| 202 | B | C |
| 203 | C | C |
| 204 | B | C |
| 205 | B | C |
| 206 | B | C |
| 207 | B | C |
| 208 | B | C |
| 209 | C | C |
| 210 | A | C |
| 211 | A | C |
| 212 | C | C |
| 213 | B | C |
| 214 | B | C |
| 215 | C | C |
| 216 | C | C |
| 217 | B | C |
| 218 | B | C |
| 219 | C | C |
| 220 | — | B |
| 221 | A | B |
| 222 | B | C |
| 223 | B | C |
| 224 | C | C |
| 225 | B | C |
| 226 | C | C |
| 227 | B | C |
| 228 | C | C |
| 229 | C | C |
| 230 | B | C |
| 231 | C | C |
| 232 | C | C |
| 233 | C | C |
| 234 | A | B |
| 235 | A | B |
| 236 | A | C |
| 237 | C | C |
| A1 | C | C |
| A2 | C | C |
| A3 | A | C |
| A4 | C | C |
| A5 | C | C |
| A6 | C | C |
| A8 | C | C |
| A10 | C | C |
| A11 | C | C |
| A12 | C | C |
| A13 | B | C |
| A14 | C | C |
| A15 | B | C |
| A16 | C | C |
| A22 | C | C |
| A24 | B | — |
| A26 | C | C |
| A27 | C | C |
| A32 | C | C |
| A34 | C | C |
| A35 | C | C |
| A36 | C | C |
| A37 | A | B |
| A38 | A | A |
| A39 | A | A |
| A40 | A | C |
| A41 | B | C |
| A43 | B | C |
| A44 | B | C |
| A45 | A | B |
| A46 | A | C |
| A47 | — | B |
| A48 | B | C |
| A49 | B | C |
| A50 | B | C |
| A51 | B | B |
| A52 | C | C |
| A53 | C | C |
| A54 | C | C |
| A55 | C | C |
| A57 | C | C |
| A58 | C | C |
| A62 | B | C |
| A63 | C | C |
| A64 | C | C |
| A65 | B | C |
| A66 | C | C |
| A67 | C | C |
| A68 | C | C |
| A70 | A | B |
| A72 | B | — |
| A73 | B | C |
| A74 | B | C |
| A75 | B | C |
| A76 | B | C |
| A77 | C | C |
| A78 | C | C |
| A80 | A | C |
| A84 | B | C |
| A85 | A | C |
| A87 | B | — |
| A89 | A | B |
| A91 | A | C |
| A92 | A | C |
| A93 | — | C |
| A94 | — | C |
| A95 | B | C |
| A96 | B | C |
| A97 | C | C |
| A98 | B | C |
| A99 | C | C |
| A100 | B | C |
| A111 | B | C |
| A102 | A | C |

“—”: not tested

### Example 5: Recalcification Time

1. Materials and Instruments

Isoflurane (Hana Pharm, cat. #9008), Isotonic Sodium Chloride Injection (Daihan Pharm, cat. #331), Citrate-dextrose solution (Sigma-Aldrich, cat. #C₃₈₂₁), Calcium chloride solution (Sigma-Aldrich, cat. #21115), 22G Syringe 10 mL (Koreavaccine), Dimethyl sulfoxide (Sigma-Aldrich, cat. #D5879), Apixaban (AK scientific, cat. #X1060), 96 Well Cell Culture Plates (SPL Life Sciences, cat. #30096), Synergy H₄ Hybrid Microplate Reader (BioTek), Combi R515 Multi-purpose Centrifuge (Hanil Scientific), BS-06 Shaking & Heating Baths (JEIO TECH), and PST-60HL-4 Plate Shaker-Thermostat (Bio-San)

2. Animals

Seven-week-old male SD Rat (ORIENT BIO.) were maintained under controlled conditions of temperature (22±2° C.) and 12-h light/12-h dark cycle. Rats were housed in the pathogen-free facility of the Laboratory Animal Research Center in Ildong Pharmaceutical Co. Ltd. All procedures on animals were conducted in accordance with the relevant national regulatory guidelines and individual experiments approved by the Ildong Pharmaceutical Co. Ltd., Institutional Animal Care and Use Committee (IACUC) (approval No.: A2106-3, 2108-4).

3. Assay Procedure

The ability of test compounds to interfere with plasma coagulation, in the presence of platelets, was analyzing by measuring the recalcification time of rat plasma. The blood of animals was collected into 15 mL tubes containing Citrate-dextrose solution (9:1 v/v) before performing the assays. For recalcification time, platelet-rich plasma (PRP) samples were obtained by centrifugation at 360×g for 10 minutes. And the PRP were preincubated for 5 minutes (37° C.). In a 96 well microplate, test compounds (2 μL) were treated in 98 μL of platelet rich plasma. After mixing with a pipette, it was incubated at 37° C. for 15 minutes. The reaction was started by addition of 200 μL of pre-warmed CaCl₂) (16 mM). Immediately, put the microplate in a microplate reader and the signals were measured by the following steps.

(1) 405 nm wavelength absorbance signals were recorded in every 10 seconds at 37° C.

(2) Basal absorbance signals were recorded for 0 second.

The signal variance of each of the test compounds was obtained by the following step.

(1) Background signals (t=O) were subtracted from recording values, and the resulting values were converted to change values. The values at 0 second were set to be zero.

The retardation time of test compound were calculated by the following formula:

Retardation time (sec)=[log EC₅₀ (test sample)−log EC₅₀ (DMSO control)]

As described above, a DMSO control group is a group to which 2% DMSO was treated.

The assay result is shown in Table 5, where compounds (30 μM) having less than 20 seconds of retardation time are marked by ‘C’; the compounds (30 μM) having retardation time of 20 seconds or more to less than 50 seconds are marked by ‘B’; and the compounds (30 μM) having retardation time of 50 seconds or more are marked by ‘A’.

TABLE 5
Recalcification Time Result

| Cmpd No. | Inhibition Level |
|---|---|
| 68 | A |
| 79 | C |
| 80 | C |
| 118 | C |
| 119 | C |
| 122 | B |
| 123 | B |
| 128 | A |
| 141 | A |
| 142 | A |
| 154 | A |
| 155 | A |
| 160 | B |
| 161 | A |
| 164 | C |
| 165 | C |
| 170 | B |
| 171 | C |
| 172 | C |
| 173 | B |
| 174 | B |
| 175 | B |
| 176 | C |

### Example 6: NATEM

1. Materials and Instruments

Star-TEM reagent (TEM International GmbH, cat. #000503-01), Dimethyl sulfoxide (Sigma-Aldrich, cat. #D2438), Cleancle normal saline (JW Pharm), and ROTEM® delta system (TEM International GmbH).

2. Blood

Human blood research was experimented after approval from the Yonsei University Institutional Review Board (IRB) in accordance with the Life Ethics and Safety Act.

3. Assay Procedure

Blood samples for NATEM were kept at room temperature, and all NATEM analyses were performed at 37° C. NATEM was carried out using 300 μL of citrated human blood preincubating with test compounds (100 μM, 2% v/v DMSO) for 15 minutes in cuvette and reversed with Star-TEM reagent according to the manufacturer's manual (ROTEM; Tem International GmbH, Munich, Germany). After addition of the star-TEM reagent, cuvettes were measured by ROTEM® delta system. Blood clot formation was evaluated.

The assay result is shown in Table 6. The obtained parameters were time to clot initiation (Clotting Time, CT), time to clot formation, α angle for clot growth kinetics (initial rate of fibrin polymerization), amplitude (firmness) at 10 minutes, maximum clot firmness, and maximum lysis.

TABLE 6
NATEM Result

| | 1^{st} experiment | | 2^{nd} experiment | |
|---|---|---|---|---|
| | 2% | Compound | 2% | Compound |
| | DMSO | 155 | DMSO | 174 |
| CT (sec) | 516 | 616 | 517 | 665 |
| CFT (sec) | 144 | 168 | 141 | 180 |
| α (°) | 62 | 59 | 63 | 57 |
| A10 (mm) | 46 | 45 | 44 | 45 |
| MCF (mm) | 59 | 57 | 57 | 56 |
| ML (%) | 1 | 2 | 1 | 2 |

CT, clotting time;
CFT, clot formation time;
α, α-angle; A5, amplitude (firmness) at 10 minutes;
MCF, maximum clot firmness;
ML, maximum lysis

1. A compound of Formula (I), a pharmaceutically acceptable salt of the compound, a solvate of the compound, or a hydrate of the compound: 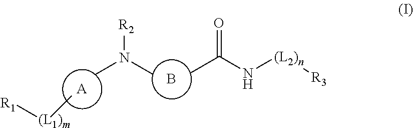
wherein ring A and ring B each are independently a monocyclic aliphatic ring, a polycyclic aliphatic ring, a monocyclic aromatic ring, or a polycyclic aromatic ring, which optionally contains at least one heteroatom selected from the group consisting of N, NO, NO₂, S, SO, SO₂, and O, wherein the ring A and ring B each are optionally and independently substituted with at least one substituent selected from the group consisting of halogen, halogen derivatives, CN, alkoxy, carboxyl, carbonyl, ester, hydroxyl, amine, amide, nitro, phosphate, thioalkyl, sulfhydryl, oxo, aliphatic, cycloaliphatic, hetero cycloaliphatic, aromatic, hetero aromatic, and aryl aliphatic;
wherein R₁ and R₃ each are independently hydrogen, halogen, halogen derivatives, CN, alkoxy, carboxyl, carbonyl, hydroxyl, amine, amide, nitro, phosphate, thioalkyl, sulfhydryl, oxo, aliphatic, cycloaliphatic, hetero cycloaliphatic, aromatic, hetero aromatic, or aryl aliphatic, wherein R₁ and R₃ each are optionally and independently substituted with at least one substituent selected from the group consisting of halogen, halogen derivatives, CN, alkoxy, carboxyl, carbonyl, ester, hydroxyl, amine, amide, nitro, phosphate, thioalkyl, sulfhydryl, oxo, aliphatic, cycloaliphatic, hetero cycloaliphatic, aromatic, hetero aromatic, and aryl aliphatic;
wherein R₂ is hydrogen, C₁₋₅ alkyl or C₃₋₆ cycloalkyl;
wherein L₁ and L₂ each are independently C₁-C₁₀ aliphatic, C₃-C₁₀ cycloaliphatic, or C₃-C₁₀ hetero cycloaliphatic, wherein L₁ and L₂ each are optionally and independently substituted with at least one substituent selected from the group consisting of CN, C₁₋₅ alkyl, and C₃₋₆ cycloalkyl; and
wherein m and n each are independently 0 or 1.

2. The compound, salt, solvate, or hydrate of claim 1,
wherein the ring A and ring B each are independently a 5-membered ring or a 6-membered ring.

3. The compound, salt, solvate, or hydrate of claim 1,
wherein the ring A is a monocyclic or polycyclic aliphatic ring which optionally contains at least one heteroatom selected from the group consisting of N, NO, NO₂, S, SO, SO₂, and O, or a monocyclic or polycyclic aromatic ring which optionally contains at least one heteroatom selected from the group consisting of N, NO, NO₂, S, SO, SO₂, and O; and/or
wherein the ring B is a monocyclic or polycyclic aliphatic ring which optionally contains at least one heteroatom selected from the group consisting of N, O, and S, or a monocyclic or polycyclic aromatic ring which optionally contains at least one heteroatom selected from the group consisting of N, O, and S.

4. The compound, salt, solvate, or hydrate of claim 1,
wherein the ring A is phenyl, pyridinyl, diazinyl, pyrimidinyl, triaziny, piperidinyl, oxadiazoline, cyclopropyl, cyclobutyl, cyclopentyl, or cyclohexyl.

5. The compound, salt, solvate, or hydrate of claim 1,
wherein the ring B is a phenyl, pyridinyl, diazinyl, cyclopentadienyl, cyclopentyl, cyclohexyl, adamantane, or bicyclo[2.2.1]heptane.

6. The compound, salt, solvate, or hydrate of claim 1,
wherein the ring A is 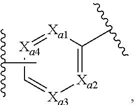
in which Xₐ₁, Xₐ₂, Xₐ₃, and Xₐ₄ each are independently CH, N, or NH, or
wherein the ring A is 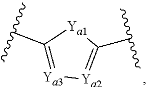
in which Yₐ₁, Yₐ₂, and Yₐ₃ each are independently CH, N, NH, S, SH or O.

7. The compound, salt, solvate, or hydrate of claim 1,
wherein the ring B is 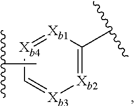
in which X_{b1}, X_{b2}, X_{b3}, and X_{b4} each are independently CH, N, or NH.

8. The compound, salt, solvate, or hydrate of claim 1,
wherein R₁ is hydrogen; C₁₋₁₀ alkyl; benzyl; alkoxy, CN, COOH, mono or bi aromatic ring which optionally contains at least one heteroatom selected from the group consisting of N, O, and S; mono or bi cycloaliphatic which optionally contains at least one heteroatom selected from the group consisting of N, O, and S; aryl which optionally contains at least one hetero atom selected from the group consisting of N, O, and S; an aromatic ring fused to a non-aromatic ring which optionally contains at least one heteroatom selected from the group consisting of N, O, and S; or an aromatic ring fused to an aromatic ring which optionally contains at least one heteroatom selected from the group consisting of N, O, and S.

9. The compound, salt, solvate, or hydrate of claim 1, wherein R₁ is C₁₋₄ alkyl, benzyl, phenyl, pyridinyl, diazinyl, triazinyl, piperidinyl, furanyl, cyclopropyl, cyclobutyl, cyclopentyl, cyclohexyl or thiophenyl, which is optionally substituted with at least one substituent selected from the group consisting of halogen, halogen derivatives, alkyl, alkenyl, alkynyl, cycloalkyl, cycloalkenyl, cycloalkynyl, hetero cycloalkyl, hetero cycloalkenyl, hetero cycloalkynyl, alkoxy, aryl, aryloxy, diaryl, arylalkyl, arylalkyloxy, cycloalkylalkyl, cycloalkylalkyloxy, amino, hydroxy, hydroxyalkyl, acyl, heteroaryl, heteroaryloxy, heteroarylalkyl, heteroarylalkoxy, aryloxyalkyl, alkylthio, arylalkylthio, aryloxyaryl, alkylamido, alkanoylamino, arylcarbonylamino, nitro, cyano, thiol, haloalkyl, trihaloalkyl, alkyl ester, and alkylthio.

10. The compound, salt, solvate, or hydrate of claim 1, wherein the ring A, R₁, or both comprise a hetero aromatic ring which contains at least one N as the heteroatom.

11. The compound, salt, solvate, or hydrate of claim 1, wherein the R₃ is hydrogen; halogen; halogen derivatives; CN; alkoxy, carboxyl, carbonyl, hydroxyl, amine, amide, nitro, phosphate, thioalkyl, sulfhydryl, oxo, aliphatic, cycloaliphatic, hetero cycloaliphatic, aromatic, hetero aromatic, aryl aliphatic or fused ring.

12. The compound, salt, solvate, or hydrate of claim 1, wherein the 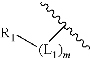
group is one of the following groups:

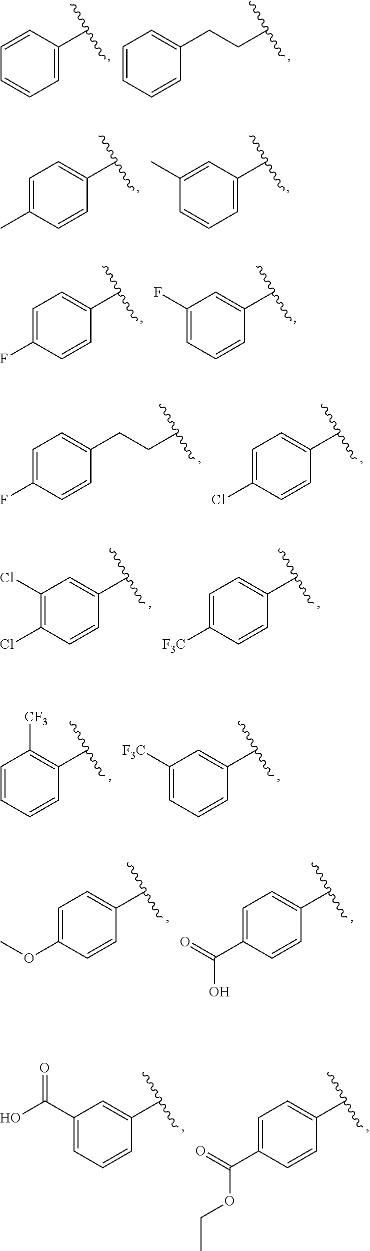

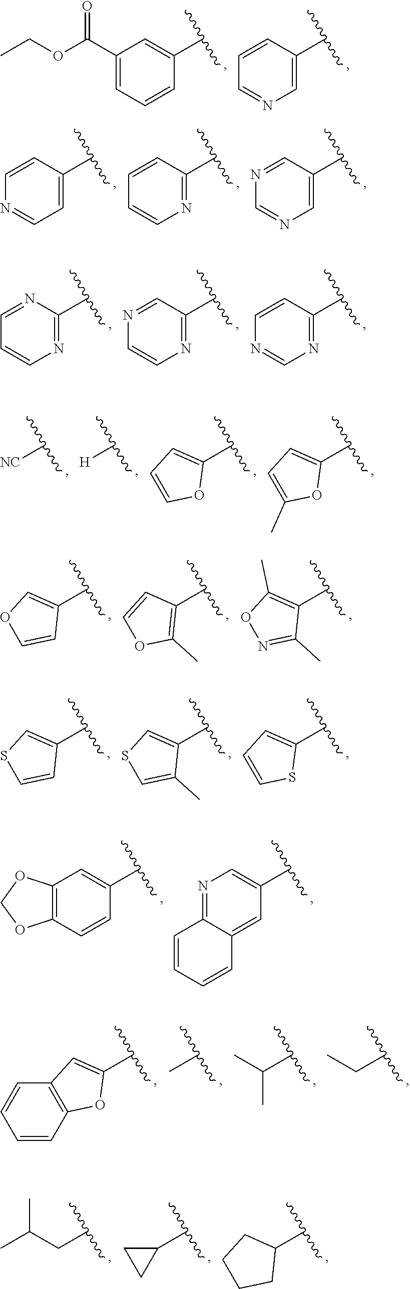

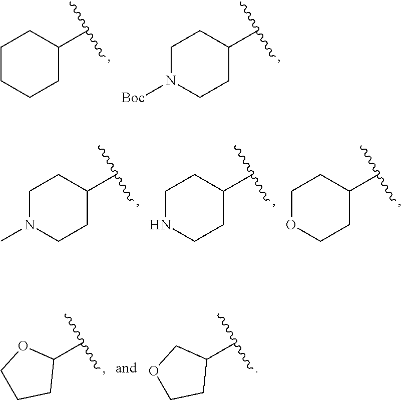

13. The compound, salt, solvate, or hydrate of claim 1, wherein the 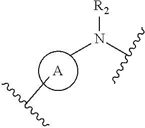
group is one of the following groups:

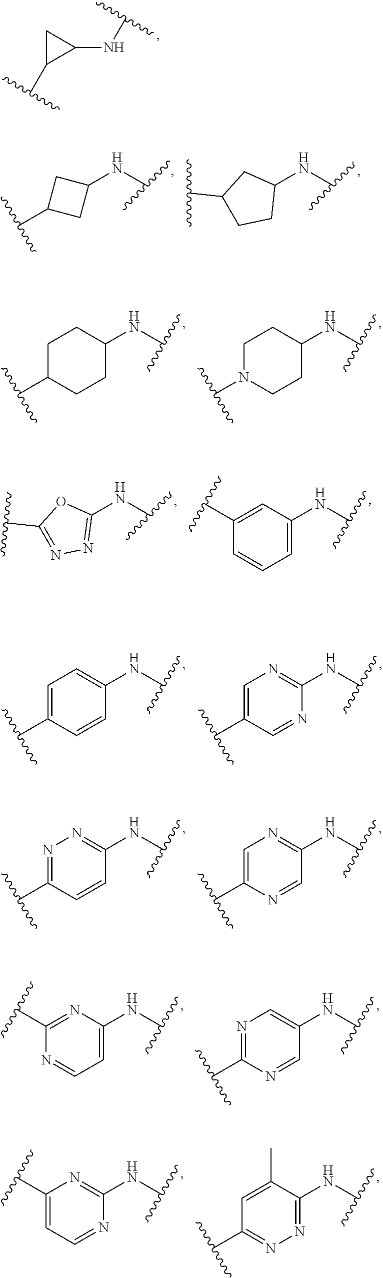

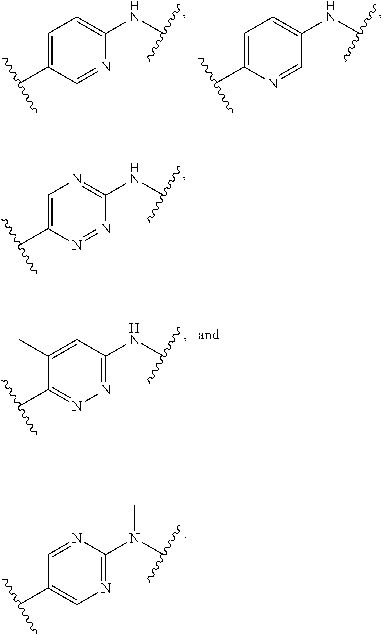

14. The compound, salt solvate, or hydrate of claim 1, wherein the 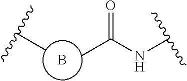
group is one of the following groups:

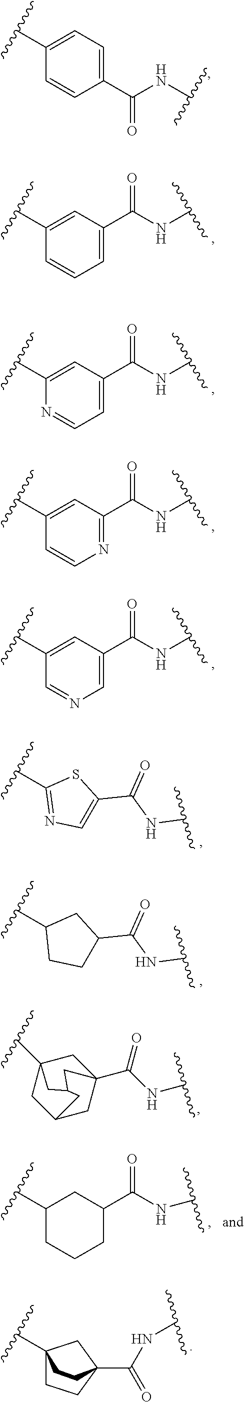

15. The compound, salt, solvate, or hydrate of claim 1, wherein the 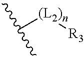
group is one of the following groups:

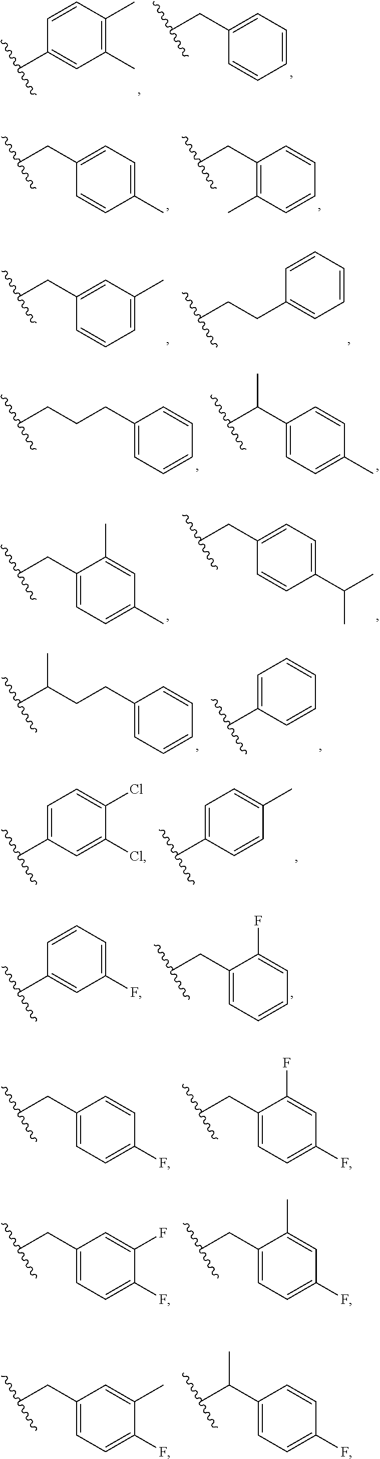

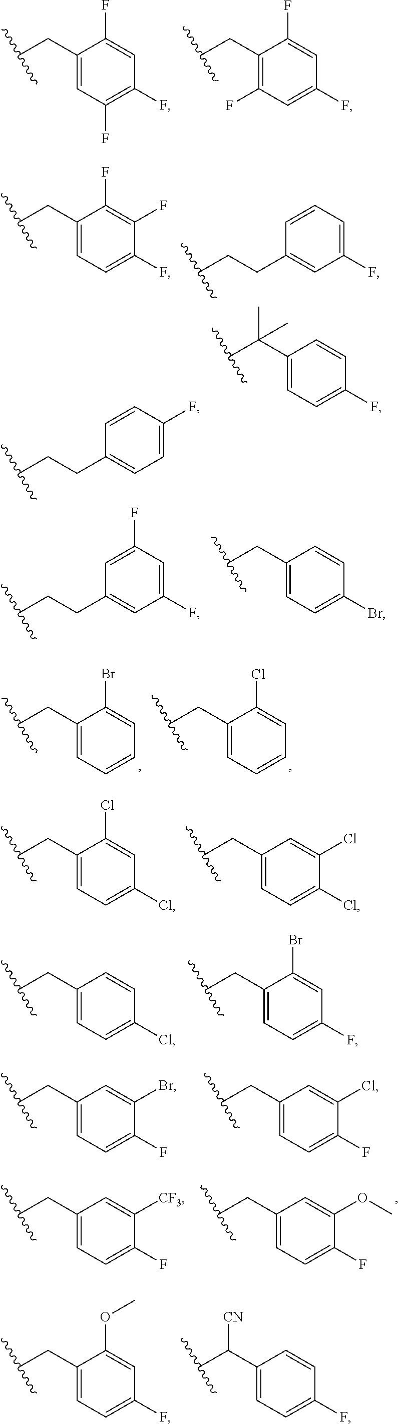

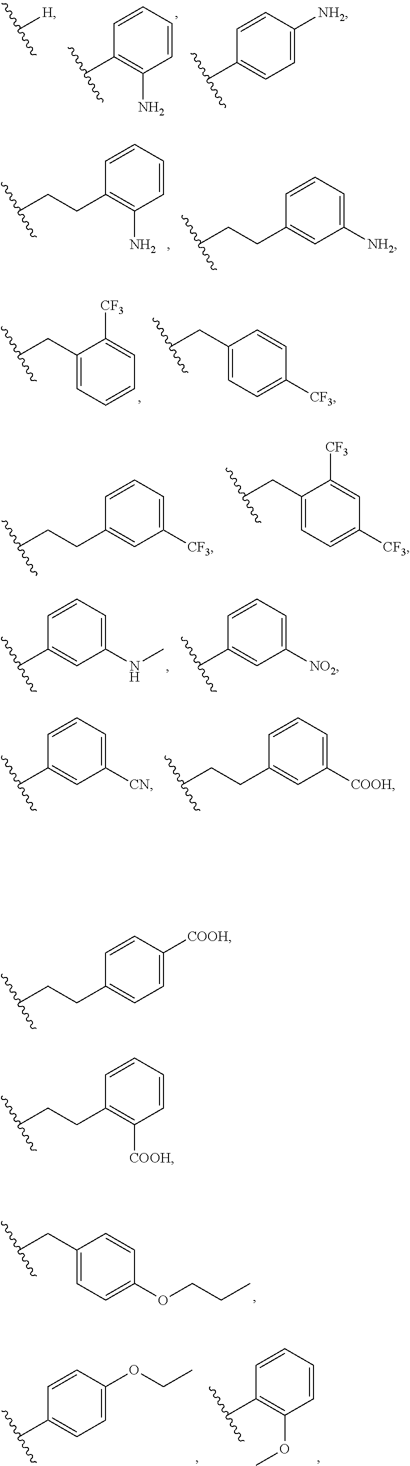

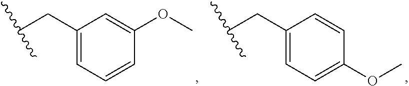

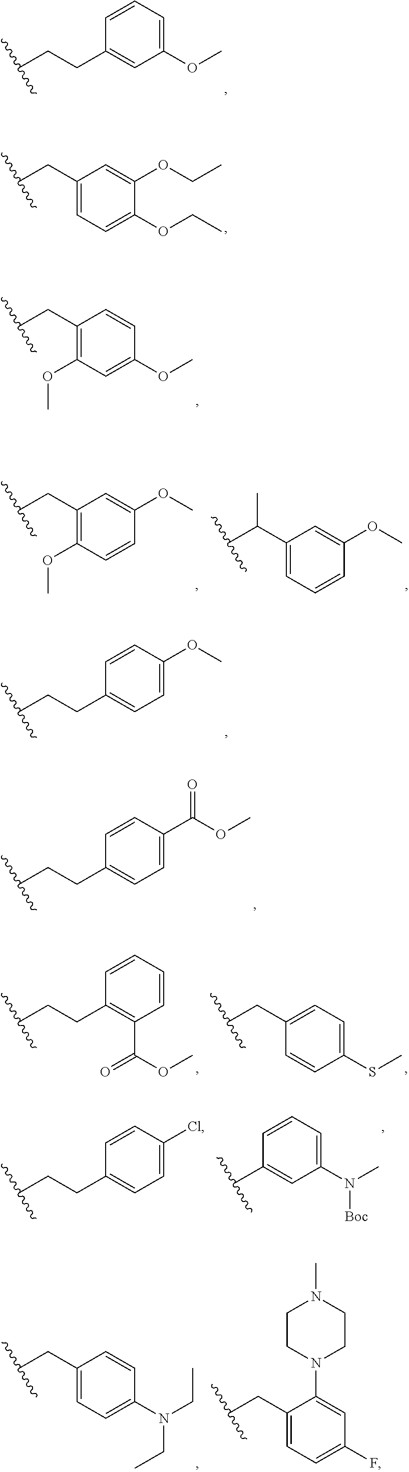

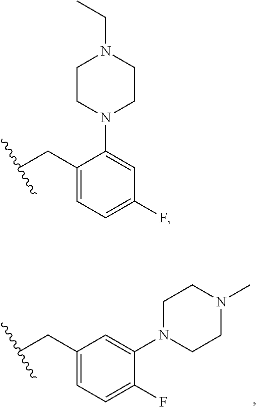 [SMILES: *Cc1ccc(F)cc1N1CCN(CC)CC1 ; *Cc1ccc(F)c(N2CCN(C)CC2)c1]

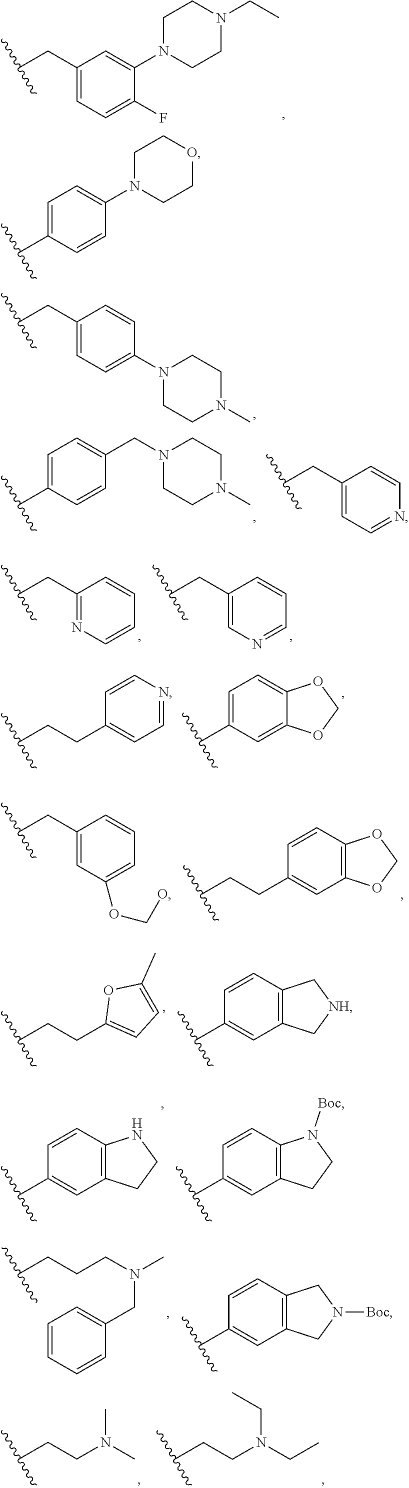

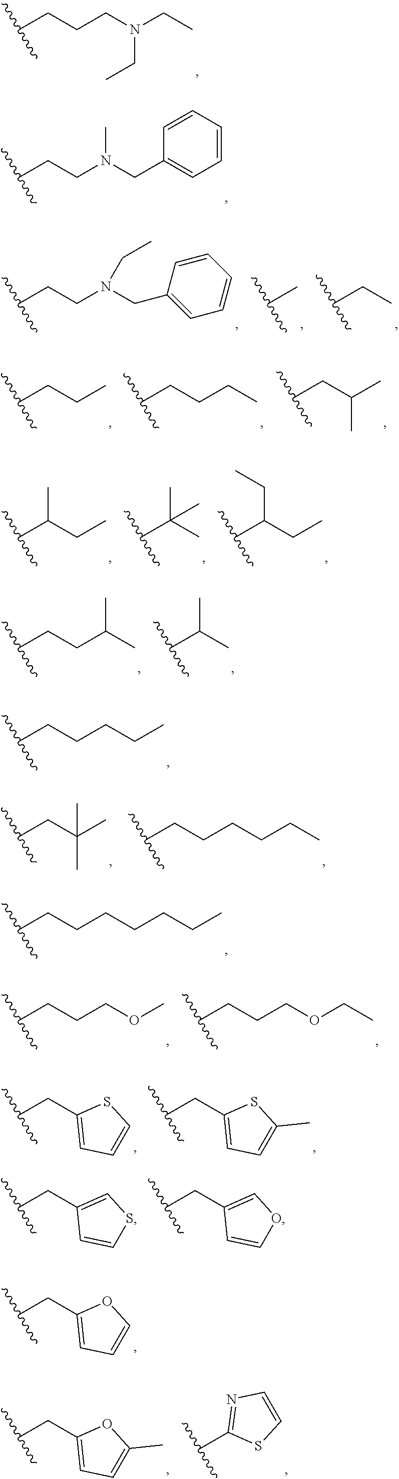

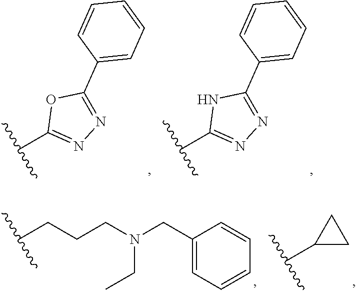

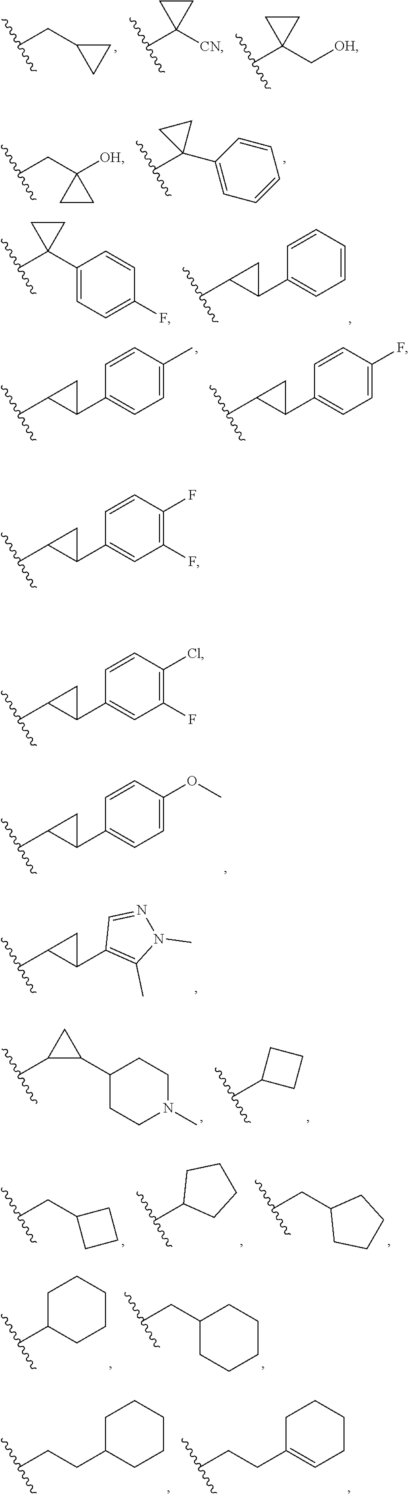

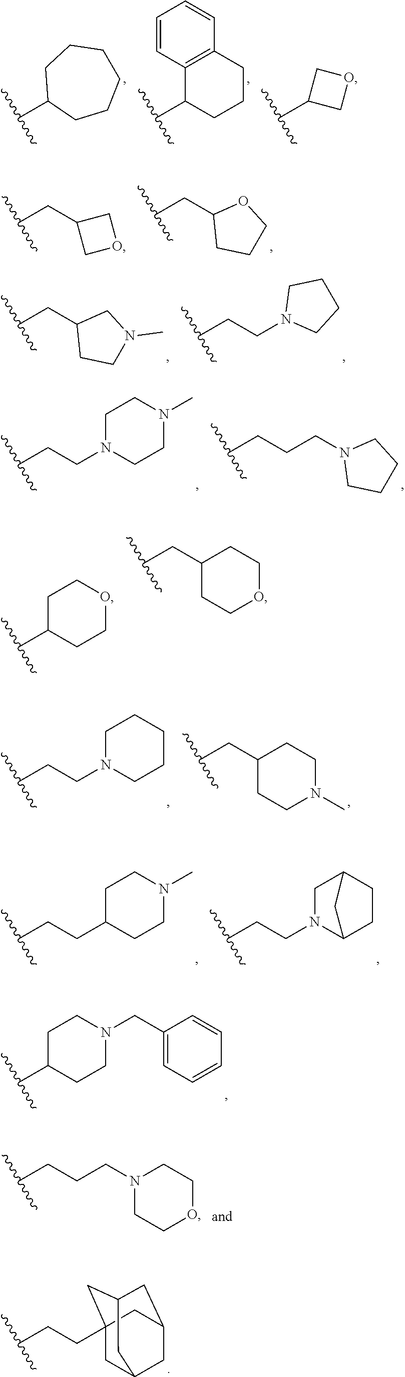

16. The compound, salt, solvate, or hydrate of claim 1, wherein the compounds of Formula (I) do not include the compounds listed in Table 2.

17. A pharmaceutical composition comprising the compound, salt, solvate, or hydrate of claim 1.

18. A method of treating or preventing disease, disorder, or condition, comprising administering to a subject in need a therapeutically effective amount of a compound, salt, solvate, or hydrate of claim 1 or a combination thereof; or administering to a subject in need a therapeutically effective amount of a composition comprising the compound, salt, solvate, hydrate, or a combination thereof of claim 1, wherein the disease, disorder, or condition is associated with anoctamin 6 (ANO6) activity, function of ion channels and/or function of phospholipid scrambling.

19. The method of claim 18, wherein the compound of claim 1 is one of the compounds listed in Table 1 or Table 2.

20. The method of claim 16, wherein the disease, disorder, or condition is thromboembolic disorder, inflammatory disease, or cancer.
